(19) United States
(12) Patent Application Publication
(10) Pub. No.: US 2026/0055387 A1
(43) Pub. Date: Feb. 26, 2026

(54) EFFECTOR PROTEINS, COMPOSITIONS, SYSTEMS AND METHODS OF USE THEREOF

(71) Applicant: Mammoth Biosciences, Inc., Brisbane, CA (US)

(72) Inventors: James Paul BROUGHTON, South San Francisco, CA (US); Adam L. GARSKE, Palo Alto, CA (US); Carley Gelenter HENDRIKS, Burlingame, CA (US); Ishita JAIN, San Francisco, CA (US); Srijay RAJAN, South San Francisco, CA (US); Isaac Zepeda MADRID, San Francisco, CA (US)

(21) Appl. No.: 19/282,985

(22) Filed: Jul. 28, 2025

Related U.S. Application Data
(63) Continuation of application No. PCT/US24/13609, filed on Jan. 30, 2024.
(60) Provisional application No. 63/482,261, filed on Jan. 30, 2023.
(60) Provisional application No. 63/586,844, filed on Sep. 29, 2023.

(51) Int. Cl. C12N 9/22; A61K 31/7088; A61K 38/46; C12N 15/113; C12Q 1/6809; C12Q 1/6823

(52) U.S. Cl. CPC C12N 9/222; A61K 31/7088; A61K 38/465; C12N 15/113; C12Q 1/6809; C12Q 1/6823; C12N 2310/20; C12Q 2600/112

(57) ABSTRACT

Provided herein are compositions, systems, devices, kits, and methods comprising effector proteins, and uses thereof. These effector proteins may be characterized as CRISPR-associated (Cas) proteins. Various compositions, systems, devices, kits, and methods of the present disclosure may leverage the activities of these effector proteins for the modifying, detecting and/or engineering of nucleic acids.

### CROSS-REFERENCE

This application is a continuation of International Patent Application No. PCT/US2024/013609, filed Jan. 30, 2024, which claims benefit of U.S. Provisional Application No. 63/482,261, filed on Jan. 30, 2023, and of U.S. Provisional Application No. 63/586,844, filed on Sep. 29, 2023, the disclosures of each of which are incorporated herein by reference in their entirety.

### INCORPORATION BY REFERENCE OF SEQUENCE LISTING

The instant application contains a Sequence Listing, which has been submitted via Patent Center. The Sequence Listing titled 203477-771301_US_SL.xml, which was created on Jul. 17, 2025, and is 478,007 bytes in size, is hereby incorporated by reference in its entirety.

### FIELD

The present disclosure relates generally to polypeptides, such as effector proteins, compositions of such polypeptides and guide nucleic acids, systems, devices, kits, and methods of using such polypeptides and compositions, including detecting and modifying target nucleic acids.

### BACKGROUND

Clustered Regularly Interspaced Short Palindromic Repeats (CRISPR) and associated proteins (Cas proteins), sometimes referred to as a CRISPR/Cas system, were first identified in certain bacterial species and are now understood to form part of a prokaryotic acquired immune system. CRISPR/Cas systems provide immunity in bacteria and archaea against viruses and plasmids by targeting the nucleic acids of the viruses and plasmids in a sequence-specific manner. While CRISPR/Cas proteins are involved in the acquisition, targeting and cleavage of foreign DNA or RNA, the systems may also contain a CRISPR array, which includes direct repeats flanking short spacer sequences that, in part, guide Cas proteins to their targets. The discovery of CRISPR/Cas systems has revolutionized the field of genomic manipulation and engineering. Yet, the discovery suffers from several shortcomings that restricts its use for basic biomedical research and therapeutic applications. In particular, compositions, systems, devices, kits, and methods for detecting and modifying target nucleic acids that may be associated with a disease or disorder still need to be developed. While the programmable nature of these systems has promising implications in the field of genome engineering, there remains a need to explore alternative strategies and components to leverage the CRISPR-Cas system in ways that are efficient for in vitro detection and effective for in vivo engineering. Effector proteins, guide nucleic acids, compositions, systems, devices, kits, and methods described herein satisfy this need and provides related advantages.

### SUMMARY

The present disclosure provides for polypeptides, such as effector proteins, compositions, systems, devices, kits, and methods comprising the same, and uses thereof. In general, compositions, systems, devices, kits, and methods comprise guide nucleic acids or uses thereof. Compositions, systems, devices, kits, and methods disclosed herein may leverage nucleic acid modifications. In some embodiments, target nucleic acid modifications described herein are initiated by the recognition of and/or cleaving of a target nucleic or non-target nucleic acid by an effector protein. In some embodiments, modifications of target nucleic acids result in certain measurable features, such as a detectable signal, silencing of gene expression, RNA degradation, and/or alternative splicing of a target nucleic acid. In some embodiments, effector proteins described herein exhibit nucleic acid cleavage activity. In some embodiments, nucleic acid cleavage activity comprises cis cleavage activity, trans cleavage activity, nicking activity, and/or nuclease activity. In some embodiments, a catalytically inactive variant of an effector protein is fused to a fusion partner, wherein a catalytically inactive variant of an effector protein and a guide nucleic acid function to recruit the fusion partner to a target nucleic acid where the fusion partner modifies the target nucleic acid or expression thereof. In some embodiments, compositions, systems, devices, kits, and methods are useful for modifying the nucleotide sequence of a target nucleic acid. In some embodiments, modifying comprises cleaving a target nucleic acid. In some embodiments, modifying comprises cleaving a non-target nucleic acid. In some embodiments, compositions, systems, devices, kits, and methods are useful for the detection of target nucleic acids. In some embodiments, compositions, systems, devices, kits, and methods are useful for the detection and/or treatment of a disease or disorder. The disease or disorder may be associated with a target nucleic acid. The disease or disorder may be associated with one or more mutations in the target nucleic acid.

### I. Certain Embodiments

The present disclosure provides compositions, systems, devices, kits, and methods comprising effector proteins and uses thereof. Compositions, systems, devices, kits, and methods disclosed herein leverage nucleic acid modifying activities (e.g., cis cleavage activity and trans cleavage activity) of these effector proteins for the modification, detection, and engineering of target nucleic acids.

Provided herein are systems comprising an engineered polypeptide, or a recombinant nucleic acid encoding the engineered polypeptide, wherein the engineered polypeptide comprises an amino acid sequence that is at least 85% identical to any one of the sequences set forth in TABLE 1.

Provided herein are systems comprising: (i) a polypeptide, or a recombinant nucleic acid encoding the polypeptide, wherein the polypeptide comprises an amino acid sequence that is at least 85% identical to any one of the sequences set forth in TABLE 1; and (ii) an engineered guide nucleic acid or a nucleic acid that encodes the engineered guide nucleic acid. In some embodiments, the polypeptide comprises an amino acid sequence that is at least 85% identical to any one of sequences SEQ ID NO: 1-7, 9, 11-15, 68-73, 76-87, and 94-99 listed in TABLE 1. In some embodiments, the polypeptide comprises an amino acid sequence that is at least 89% identical to SEQ ID NO: 88, at least 91% identical to SEQ ID NO: 89, at least 92% identical to SEQ ID NO: 90 and 92, at least 93% identical to SEQ ID NO: 74-75, at least 94% identical to SEQ ID NO: 91, at least 98% identical to SEQ ID NO: 8, or at least 99.5% identical to SEQ ID NO: 10.

Also provided herein are systems comprising a polypeptide, or a recombinant nucleic acid encoding the polypeptide, wherein the polypeptide comprises a variant amino acid sequence of SEQ ID NO: 69, or a functional fragment thereof, wherein the variant amino acid sequence comprises one or more amino acid alterations at one or more residues corresponding to one or more positions listed in TABLE 1.1; and optionally wherein the amino acid sequence, other than the one or more amino acid alterations, has at least 85% sequence identity to the amino acid sequence referenced in SEQ ID NO: 69. In some embodiments, the one or more amino acid alterations are individually at one or more residues corresponding to one or more positions selected from: 9, 15, 56, 106, 121, 125, 131, 139, 150, 154, 164, 166, 175, 184, 198, 200, 242, 247, 262, 265, 281, 289, 305, 311, 313, 314, 318, 333, 338, 352, 372, 381, 480, 485, 492, 496, 501, 517, 521, 537, 543, 546, 547, 548, 555, 559, 567, 569, 574, 579, 585, 618, 621, 622, 623, 631, 647, 656, 684, 705, 709, 717, 722, 726, 737, 747, 762, 765, 766, 769, 789, 790, 800, 801, 807, 819, 827, 836, 843, 846, 847, 857, 858, 864, 867, 870, 871, 909, 915, 919, 923, 927, 974, 1011, 1020, 1030, 1032, 1035, 1049, 1054, 1056, 1062, 1064, 1083, 1085, or combinations thereof, relative to SEQ ID NO: 69. In some embodiments, the one or more amino acid alterations are individually at one or more residues corresponding to one or more positions selected from: 121, 139, 311, 184, 154, 547, 318, 656, 372, 858, 548, 352, 927, 737, 1062, 819, 501, 974, 1064, 722, 621, 765, 622, 807, 762, 871, 800, 827, 1020, or combinations thereof, relative to SEQ ID NO: 69. In some embodiments, the one or more amino acid alterations are each a substitution of an amino acid residue with a basic (positively charged) amino acid, an acidic (negatively charged) amino acid, a non-polar (hydrophobic) amino acid, an uncharged polar amino acid, or combinations thereof. In some embodiments, the one or more amino acid alterations are each a substitution of an amino acid residue with an amino acid residue selected from a group comprising: Gly (G), Lys (K), Ala (A), Gln (Q), Asn (N), Leu (L), Tyr (Y), Arg (R), Glu (E), Met (M), Thr (T), Val (V), Ser (S), His (H), Ile (I), Cys (C), Pro (P), Asp (D), or combinations thereof. In some embodiments, the one or more amino acid alterations are each a substitution of an amino acid residue with an amino acid residue selected from a group comprising: Asn (N), Gln (Q), Val (V), Glu (E), Lys (K), Leu (L), Ala (A), Cys (C), Ile (I), Ser (S), Pro (P), Thr (T), Tyr (Y), Arg (R), Gly (G), or combinations thereof. In some embodiments, each of the one or more amino acid alterations are individually selected from a group comprising: T9G, T15K, Q56A, H106Q, E121N, C125L, E131K, H139Q, N150Y, G154K, H164R, Q166K, Q175K, D184E, E198K, F200Y, A242M, R247T, A262T, N265R, N281K, D289T, H305K, M311V, A313S, N314K, K318Q, E333H, L338I, S352C, V372L, L381M, Q480Y, Q485S, V492Q, H496S, V501T, G517N, S521A, L537R, E543L, W546Y, S547N, G548A, I555L, Y559N, N567S, D569H, D574Q, L579V, Q585L, Q618E, W621Q, I622N, I622K, M623L, D631C, L647V, M656L, L684K, A705T, Q709H, K717M, N722R, T726Q, A737S, T747L, A762G, W765R, W765N, Q766M, K769E, T789Q, N790K, D800K, D800G, D800R, E801M, E801G, S807T, S807R, S819K, S827R, S827K, N836P, N843S, A846P, T847K, E857M, Y858L, E864Q, E867A, D870E, N871K, N909D, N909E, E915V, S919Q, S919K, I923L, M927I, D974Y, L1011V, H1020R, A1030S, T1032V, D1035M, D1049G, Q1054T, Q1056L, S1062P, I1064K, P1083Q, A1085E, or combinations thereof, relative to SEQ ID NO: 69. In some embodiments, each of the one or more amino acid alterations are individually selected from a group comprising: E121N, H139Q, M311V, D184E, G154K, S547N, K318Q, M656L, V372L, Y858L, G548A, S352C, M927I, A737S, S1062P, S819K, V501T, D974Y, I1064K, N722R, W621Q, W765N, I622N, S807R, A762G, N871K, D800R, S827K, H1020R, or combinations thereof, relative to SEQ ID NO: 69. In some embodiments, the one or more amino acid alterations comprise: (a) E121N, M311V, S547N, M656L, Y858L, M927I, and S1062P relative to SEQ ID NO: 69; (b) H139Q, D184E, K318Q, V372L, G548A, and A737S relative to SEQ ID NO: 69; (c) H139Q, D184E, K318Q, V372L, G548A, A737S, S819K, D974Y, and I1064K relative to SEQ ID NO: 69; (d) H139Q, D184E, M311V, K318Q, S352C, V372L, V501T, G548A, N722R, A737S, W765N, S807R, S819K, N871K, D974Y, and I1064K relative to SEQ ID NO: 69; (e) H139Q, G154K, D184E, M311V, K318Q, S352C, V372L, V501T, G548A, I622N, N722R, A737S, W765N, S807R, S819K, S827K, N871K, D974Y, H1020R, and I1064K relative to SEQ ID NO: 69; or (f) H139Q, D184E, M311V, K318Q, S352C, V372L, V501T, G548A, W621Q, I622N, N722R, A737S, A762G, W765N, D800R, S807R, S819K, N871K, D974Y, and I1064K relative to SEQ ID NO: 69.

Also provided herein are systems comprising a polypeptide, or a recombinant nucleic acid encoding the polypeptide, wherein the polypeptide comprises an amino acid sequence that is at least 85% identical to any one of SEQ ID NO: 119-282 listed in TABLE 1.2.

In some embodiments, the system comprising polypeptide comprises a variant amino acid sequence of SEQ ID NO: 69 as described herein further comprises an engineered guide nucleic acid or a nucleic acid that encodes the engineered guide nucleic acid.

In some embodiments, the polypeptide interacts with an engineered guide nucleic acid. In some embodiments, the engineered guide nucleic acid comprises a repeat sequence and a spacer sequence. In some embodiments, the engineered guide nucleic acid comprises a crRNA. In some embodiments, the engineered guide nucleic acid comprises a first region or sequence and a second region or sequence, wherein the second region or sequence comprises a nucleotide sequence that is complementary to a target sequence in a target nucleic acid, wherein the first region or sequence and the second region or sequence are heterologous to each other. In some embodiments, the first region or sequence is covalently linked to the 5′ end of the second region or sequence. In some embodiments, the first region or sequence comprises a repeat sequence, wherein the repeat sequence is at least 75% identical to any one of nucleotide sequences set forth in TABLE 3. In some embodiments, the engineered guide nucleic acid comprises a repeat sequence and wherein; (a) the polypeptide comprises an amino acid sequence that is at least 85% identical to SEQ ID NO: 1 and wherein the repeat sequence comprises a nucleotide sequence that is at least 75% identical to SEQ ID NO: 30 or SEQ ID NO: 50; (b) the polypeptide comprises an amino acid sequence that is at least 85% identical to SEQ ID NO: 2 and wherein the repeat sequence comprises a nucleotide sequence that is at least 75% identical to SEQ ID NO: 31, SEQ ID NO: 35, or SEQ ID NO: 47; (c) the polypeptide comprises an amino acid sequence that is at least 85% identical to SEQ ID NO: 3 and wherein the repeat sequence comprises a nucleotide sequence that is at least 75% identical to SEQ ID NO: 32 or SEQ ID NO: 39; (d) the polypeptide comprises an amino acid sequence that is at least 85% identical to SEQ ID NO: 4 and wherein the repeat sequence comprises a nucleotide sequence that is at least 75% identical to SEQ ID NO: 33 or SEQ ID NO: 44; (e) the polypeptide comprises an amino acid sequence that is at least 85% identical to SEQ ID NO: 5 and wherein the repeat sequence comprises a nucleotide sequence that is at least 75% identical to SEQ ID NO: 34, SEQ ID NO: 40, or SEQ ID NO: 48; (f) the polypeptide comprises an amino acid sequence that is at least 85% identical to SEQ ID NO: 6 and wherein the repeat sequence comprises a nucleotide sequence that is at least 75% identical to SEQ ID NO: 35 or SEQ ID NO: 31; (g) the polypeptide comprises an amino acid sequence that is at least 85% identical to SEQ ID NO: 7 and wherein the repeat sequence comprises a nucleotide sequence that is at least 75% identical to SEQ ID NO: 36, SEQ ID NO: 48, or SEQ ID NO: 52; (h) the polypeptide comprises an amino acid sequence that is at least 85% identical to SEQ ID NO: 8 and wherein the repeat sequence comprises a nucleotide sequence that is at least 75% identical to SEQ ID NO: 37 or SEQ ID NO: 42; (i) the polypeptide comprises an amino acid sequence that is at least 85% identical to SEQ ID NO: 9 and wherein the repeat sequence comprises a nucleotide sequence that is at least 75% identical to SEQ ID NO: 38 or SEQ ID NO: 58; (j) the polypeptide comprises an amino acid sequence that is at least 85% identical to SEQ ID NO: 10 and wherein the repeat sequence comprises a nucleotide sequence that is at least 75% identical to SEQ ID NO: 39, SEQ ID NO: 32, or SEQ ID NO: 49; (k) the polypeptide comprises an amino acid sequence that is at least 85% identical to SEQ ID NO: 11 and wherein the repeat sequence comprises a nucleotide sequence that is at least 75% identical to SEQ ID NO: 40 or SEQ ID NO: 34; (l) the polypeptide comprises an amino acid sequence that is at least 85% identical to SEQ ID NO: 12 and wherein the repeat sequence comprises a nucleotide sequence that is at least 75% identical to SEQ ID NO: 41 or SEQ ID NO: 46; (m) the polypeptide comprises an amino acid sequence that is at least 85% identical to SEQ ID NO: 13 and wherein the repeat sequence comprises a nucleotide sequence that is at least 75% identical to SEQ ID NO: 42; (n) the polypeptide comprises an amino acid sequence that is at least 85% identical to SEQ ID NO: 14 and wherein the repeat sequence comprises a nucleotide sequence that is at least 75% identical to SEQ ID NO: 43 or SEQ ID NO: 30; (o) the polypeptide comprises an amino acid sequence that is at least 85% identical to SEQ ID NO: 15 and wherein the repeat sequence comprises a nucleotide sequence that is at least 75% identical to SEQ ID NO: 44 or SEQ ID NO: 33; (p) the polypeptide comprises an amino acid sequence that is at least 85% identical to SEQ ID NO: 68 and wherein the repeat sequence comprises a nucleotide sequence that is at least 75% identical to SEQ ID NO: 48 or SEQ ID NO: 59; (q) the polypeptide comprises an amino acid sequence that is at least 85% identical to SEQ ID NO: 69 and wherein the repeat sequence comprises a nucleotide sequence that is at least 75% identical to SEQ ID NO: 57 or SEQ ID NO: 60; (r) the polypeptide comprises an amino acid sequence that is at least 85% identical to SEQ ID NO: 70 and wherein the repeat sequence comprises a nucleotide sequence that is at least 75% identical to SEQ ID NO: 61; (s) the polypeptide comprises an amino acid sequence that is at least 85% identical to SEQ ID NO: 71 and wherein the repeat sequence comprises a nucleotide sequence that is at least 75% identical to SEQ ID NO: 62; (t) the polypeptide comprises an amino acid sequence that is at least 85% identical to SEQ ID NO: 72 and wherein the repeat sequence comprises a nucleotide sequence that is at least 75% identical to SEQ ID NO: 63; (u) the polypeptide comprises an amino acid sequence that is at least 85% identical to SEQ ID NO: 73 and wherein the repeat sequence comprises a nucleotide sequence that is at least 75% identical to SEQ ID NO: 64; (v) the polypeptide comprises an amino acid sequence that is at least 85% identical to SEQ ID NO: 74 and wherein the repeat sequence comprises a nucleotide sequence that is at least 75% identical to SEQ ID NO: 65; (w) the polypeptide comprises an amino acid sequence that is at least 85% identical to SEQ ID NO: 75 and wherein the repeat sequence comprises a nucleotide sequence that is at least 75% identical to SEQ ID NO: 66; (x) the polypeptide comprises an amino acid sequence that is at least 85% identical to SEQ ID NO: 76 and wherein the repeat sequence comprises a nucleotide sequence that is at least 75% identical to SEQ ID NO: 67; (y) the polypeptide comprises an amino acid sequence that is at least 85% identical to SEQ ID NO: 93 and wherein the repeat sequence comprises a nucleotide sequence that is at least 75% identical to SEQ ID NO: 45; (z) the polypeptide comprises an amino acid sequence that is at least 85% identical to any one of SEQ ID NO: 94-97 and wherein the repeat sequence comprises a nucleotide sequence that is at least 75% identical to SEQ ID NO: 53; (aa) the polypeptide comprises an amino acid sequence that is at least 85% identical to SEQ ID NO: 97 and wherein the repeat sequence comprises a nucleotide sequence that is at least 75% identical to SEQ ID NO: 54; (bb) the polypeptide comprises an amino acid sequence that is at least 85% identical to SEQ ID NO: 98 or 99 and wherein the repeat sequence comprises a nucleotide sequence that is at least 75% identical to SEQ ID NO: 55; or (cc) the polypeptide comprises an amino acid sequence that is at least 85% identical to any one of SEQ ID NO: 90, 91, 92, 98, or 99 and wherein the repeat sequence comprises a nucleotide sequence that is at least 75% identical to SEQ ID NO: 56. In some embodiments, the first region or sequence, at least partially, interacts with the polypeptide. In some embodiments, the second region or sequence comprises a spacer sequence. In some embodiments, the spacer sequence is at least 80%, at least 85%, at least 90%, at least 95%, at least 98%, at least 99% or 100% complementary to the target sequence. In some embodiments, the engineered guide nucleic acid or a portion thereof hybridizes to a target nucleic acid. In some embodiments, the target nucleic acid is RNA. In some embodiments, the RNA is single stranded RNA, double stranded RNA, linear single-stranded RNA, circular RNA, coding RNA, non-coding RNA, or combinations thereof.

In some embodiments, the systems described herein are systems for modifying a target nucleic acid. In some embodiments, the system modifies a target nucleic acid when a complex comprising the polypeptide and an engineered guide nucleic acid hybridizes to a target sequence in a target nucleic acid. In some embodiments, the complex comprising the polypeptide and an engineered guide nucleic acid cleaves the target nucleic acid within the target sequence or within 50 nucleotides of the 5′ or 3′ end of the target sequence. In some embodiments, the complex comprising the polypeptide and an engineered guide nucleic acid cleaves a non-target nucleic acid. In some embodiments, the non-target nucleic acid is selected from a RNA and a ssDNA. In some embodiments, the engineered guide nucleic acid comprises at least 10 contiguous nucleotides that are complementary to the target sequence in the target nucleic acid. In some embodiments, the engineered guide nucleic acid comprises one or more phosphorothioate (PS) backbone modifications, 2′-fluoro (2′-F) sugar modifications, or 2′-O-Methyl (2′OMe) sugar modifications.

In some embodiments, any one of the systems described herein comprising an additional engineered guide nucleic acid, at least a portion of which hybridizes to a different target sequence of the target nucleic acid than the engineered guide nucleic acid.

In some embodiments, the polypeptide is fused to at least one heterologous polypeptide, and optionally wherein the at least one heterologous polypeptide comprises a nuclear localization signal (NLS). In some embodiments, the polypeptide comprises a length of about 800 amino acids to about 1,500 amino acids. In some embodiments, the polypeptide comprises a higher eukaryotes and prokaryotes nucleotide (HEPN) domain that is capable of cleaving a target nucleic acid. In some embodiments, the polypeptide is capable of cleaving a target nucleic acid or the polypeptide is capable of modifying at least one nucleotide of a target nucleic acid. In some embodiments, modifying comprises cleaving the target nucleic acid, including silencing, degradation, or splicing of at least one nucleotide of the target nucleic acid. In some embodiments, the polypeptide is fused to a base editing enzyme, optionally wherein the base editing enzyme comprises a deaminase. In some embodiments, modifying comprises modifying a nucleobase of at least one nucleotide of the target nucleic acid.

Also provided herein are systems for detecting a target nucleic acid, comprising any one of the systems described herein, and a reporter, wherein the reporter comprises a nucleic acid and a detectable moiety, and wherein the nucleic acid comprises RNA, ssDNA, or a combination thereof.

In some embodiments, cleavage of the reporter generates a detectable product or detectable signal from the detectable moiety. In some embodiments, cleavage of the reporter reduces a detectable signal from the detectable moiety. In some embodiments, cleavage of the reporter is effective to produce a detectable product comprising a detectable moiety. In some embodiments, the reporter is cleaved by the polypeptide. In some embodiments, the reporter is configured to release a detection moiety when cleaved by the polypeptide following hybridizing of the engineered guide nucleic acid to the target nucleic acid, and wherein release of the detection moiety is indicative of a presence or absence of the target nucleic acid.

In some embodiments, any one of the systems described herein comprising at least one detection reagent for detecting a target nucleic acid. In some embodiments, the at least one detection reagent is selected from a reporter nucleic acid, a detection moiety, an additional polypeptide, or a combination thereof, optionally wherein the reporter nucleic acid comprises a fluorophore, a quencher, or combinations thereof. In some embodiments, the at least one detection reagent is operably linked to a polypeptide, such that a detection event occurs upon contacting the system with a target nucleic acid. In some embodiments, the reporter is operably linked to a polypeptide.

In some embodiments, the engineered guide nucleic acid is capable of hybridizing to a target sequence in a target nucleic acid, and wherein the target nucleic acid is any one of: a naturally occurring eukaryotic sequence, an engineered eukaryotic sequence, a fragment of a naturally occurring eukaryotic sequence, a fragment of an engineered eukaryotic sequence, and combinations thereof.

In some embodiments, systems comprising a recombinant nucleic acid encoding a polypeptide, wherein the recombinant nucleic acid encoding the polypeptide is a nucleic acid expression vector, and optionally wherein the nucleic acid expression vector is a viral vector or an adeno associated viral (AAV) vector. In some embodiments, the nucleic acid expression vector encodes at least one engineered guide nucleic acid.

Also provided herein are systems comprising an engineered polypeptide, or a recombinant nucleic acid encoding the engineered polypeptide, wherein the engineered polypeptide comprises an amino acid sequence that is at least 85% identical to any one of the sequences set forth in TABLE 1.

Provided herein are pharmaceutical compositions, comprising any one of the systems described herein, and a pharmaceutically acceptable excipient, carrier or diluent.

Provided herein are methods of detecting a presence of a target nucleic acid in a sample, the method comprising: (a) contacting the sample with any one of the systems described herein; (b) cleaving a reporter with the polypeptide in response to formation of a complex comprising the polypeptide, an engineered guide nucleic acid, and a target sequence in a target nucleic acid, thereby producing a detectable product; and (c) detecting the detectable product, thereby detecting the presence of the target nucleic acid in the sample.

Also provided herein are methods of detecting a presence of a target nucleic acid in a sample, the method comprising: (a) contacting the sample with any one of the systems described herein; (b) cleaving a non-target sequence in a non-target nucleic acid with the polypeptide in response to formation of a complex comprising the polypeptide, an engineered guide nucleic acid, and a target sequence in a target nucleic acid, thereby producing a detectable product; and (c) detecting the detectable product, thereby detecting the presence of the target nucleic acid in the sample, wherein the target nucleic acid is amplified DNA, DNA synthesized from a single-stranded RNA template, or cDNA, wherein the non-target nucleic acid part of a reporter, and wherein the polypeptide is capable of both hybridizing to the target nucleic acid and cleaving the non-target sequence.

Provided herein are methods of modifying a target nucleic acid, the method comprising contacting the target nucleic acid with any one of the systems described herein, or any one of the pharmaceutical compositions described herein, thereby producing a modified target nucleic acid.

Also provided herein are methods of treating a disease or disorder associated with a mutation or aberrant expression of a gene in a subject in need thereof, the method comprising administering to the subject of the pharmaceutical compositions described herein.

Provided herein are systems, kits, containers, devices, or compositions comprising: (a) a polypeptide, or a nucleic acid encoding the polypeptide, and an engineered guide nucleic acid, or a nucleic acid that encodes the engineered guide nucleic acid; (b) a polypeptide, or a nucleic acid encoding the polypeptide, and an engineered guide nucleic acid comprising a crRNA; (c) an mRNA encoding a polypeptide, and an engineered guide nucleic acid; (d) one or more recombinant expression vectors comprising: i) a nucleic acid encoding a polypeptide; and ii) an engineered guide nucleic acid; or (e) one or more recombinant expression vectors comprising: i) a nucleic acid encoding a polypeptide; and ii) an engineered guide nucleic acid comprising a crRNA; wherein the polypeptide comprises an amino acid sequence that is at least 85% identical to any one of the sequences set forth in TABLE 1.

Provided herein are microfluidic devices comprising: (a) a sample interface configured to receive a sample comprising nucleic acids; and (b) a chamber fluidically connected to the sample interface; wherein the chamber comprises a polypeptide and an engineered guide nucleic acid, wherein the polypeptide comprises an amino acid sequence that is at least 85% identical to any one of the sequences set forth in TABLE 1.

In some embodiments, any one of the systems described herein, any one of the kits described herein, any one of the devices described herein, or any one of the microfluidic devices described herein, wherein components of the system, kit, device, or microfluidic device are used in diagnosis of a disease or disorder.

Also provided herein are methods for diagnosis comprising the use of any one of the systems described herein, any one of the kits described herein, any one of the devices described herein, or any one of the microfluidic devices described herein, wherein components of the system, kit, device, or microfluidic device further comprises a detectable label or a nucleic acid comprising a detectable label capable of hybridizing to a target nucleic acid.

Provided herein are polypeptides, or recombinant nucleic acids encoding the polypeptides, wherein the polypeptides comprise a variant amino acid sequence of SEQ ID NO: 69, or a functional fragment thereof, wherein the variant amino acid sequence comprises one or more amino acid alterations at one or more residues corresponding to one or more positions listed in TABLE 1.1; and optionally wherein the amino acid sequence, other than the one or more amino acid alterations, has at least 85% sequence identity to the amino acid sequence referenced in SEQ ID NO: 69.

Also provided herein are polypeptides, or recombinant nucleic acids encoding the polypeptides, wherein the polypeptide comprises an amino acid sequence that is at least 85% identical to any one of SEQ ID NO: 119-282 listed in TABLE 1.2.

In some embodiments, the polypeptide is complexed with and/or interacts with a guide nucleic acid or an engineered guide nucleic acid.

Also described herein are recombinant nucleic acids encoding the polypeptides described herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the present disclosure are utilized, and the accompanying drawings of which:

FIG. 1 shows illustrative results for exemplary effector protein detection of target nucleic acid at 37° C. with different effector proteins (SEQ ID NO: 5 or 7) in two different buffer systems (Buffer 1, or Buffer 2). Effector protein-based detection was monitored via generation of a FAM fluorescent signal.

FIG. 2 shows illustrative results for exemplary effector protein detection of target nucleic acid at 37° C. with an effector protein (SEQ ID NO: 7) with three different reporter nucleic acids (rep001, rep005, or rep045) in two different buffer systems (Buffer 1, or Buffer 2). Effector protein-based detection was monitored via generation of a FAM fluorescent signal.

FIGS. 3A-3C show illustrative results for exemplary effector protein detection of target nucleic acid at 37° C. with an effector protein (SEQ ID NO:7) with three different guide nucleic acids (e.g., crRNA) in two different buffer systems (Buffer 1, or Buffer 2) for various target nucleic acid concentrations (0 pM, 0.1 pM, 0.01 pM, 1 pM, 10 pM, or 100 pM). Effector protein-based detection was monitored via generation of a FAM fluorescent signal.

FIGS. 4A-4D show illustrative results for exemplary effector protein detection of target nucleic acid at 37° C. with different amounts of effector proteins (SEQ ID NO:69 or 51) with different guide nucleic acids (e.g., sgRNA, or crRNA) in two different buffer systems (Buffer 1, or Buffer 2). Effector protein-based detection was monitored via generation of a FAM fluorescent signal.

FIGS. 5A-5B show illustrative results for exemplary effector protein detection of target nucleic acid at 37° C. with different effector proteins (SEQ ID NO: 69 or 51) with four different reporter nucleic acids (rep001, rep005, rep045, or rep066) in two different buffer systems (Buffer 1, or Buffer 2). Effector protein-based detection was monitored via generation of a FAM fluorescent signal.

FIGS. 6A-6D show illustrative results for a cleavage assay of effector proteins (SEQ ID NO: 69 or 51), in the presence or absence of a target nucleic acid, at various temperatures (37° C., 41° C., 45° C., or 50° C.) following lysis under different lysis conditions (L1, L2, L3, LA, L5, or L6, as compared to a control condition), which result in a composition comprising 2.5% lysate. Effector protein-based detection was monitored via generation of a FAM fluorescent signal.

FIGS. 7A-7D show illustrative results for a cleavage assay of effector proteins (SEQ ID NO: 69 or 51), in the presence or absence of a target nucleic acid, at various temperatures (37° C., 41° C., 45° C., or 50° C.) following lysis under different lysis conditions (L1, L2, L3, L4, L5, or L6, as compared to a control condition), which result in a composition comprising 0.25% lysate. Effector protein-based detection was monitored via generation of a FAM fluorescent signal.

FIGS. 8A-8D show illustrative results for exemplary effector protein detection of target nucleic acid at 37° C. with effector protein (SEQ ID NO: 69) with different guide nucleic acids (e.g., crRNA having various spacer sequence lengths: 20 nt, 22 nt, 24 nt, 26 nt, or 28 nt) in buffer system Buffer 2. Effector protein-based detection was monitored via generation of a FAM fluorescent signal.

FIGS. 9A-9B show illustrative results for exemplary effector protein detection of target nucleic acid at 37° C. with two different effector proteins (SEQ ID NO: 122 or 51) for various target nucleic acid concentrations (0 copies/reaction, 1×10⁴ copies/reaction, 2×10⁴ copies/reaction, 5×10⁴ copies/reaction, 1×10⁵ copies/reaction, 2×10⁵ copies/reaction, 5×10⁵ copies/reaction, or 1×10⁶ copies/reaction). Effector protein-based detection was monitored via generation of a FAM fluorescent signal.

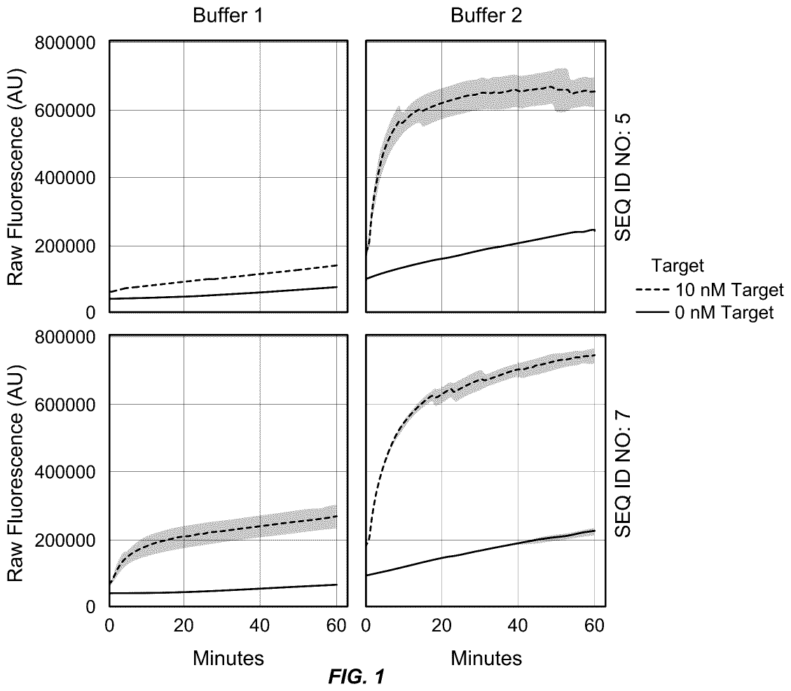

FIGS. 1A-10C shows illustrative results for exemplary effector protein detection of target nucleic acid at 37° C. with different effector proteins (SEQ ID NO: 120, 121, 122, 123, 124, or 69) for various target nucleic acid concentrations (0 pM, 0.1 pM, 0.5 pM, 1 pM, 2 pM, 5 pM, or 10 pM). Effector protein-based detection was monitored via generation of a FAM fluorescent signal.

### DETAILED DESCRIPTION

It is to be understood that both the foregoing general description and the following detailed description are exemplary, and explanatory only, and are not restrictive of the disclosure.

The section headings used herein are for organizational purposes only and are not to be construed as limiting the subject matter described.

All documents, or portions of documents, cited in this application, including, but not limited to, patents, patent applications, articles, books, and treatises, are hereby expressly incorporated by reference in their entirety for any purpose.

### II. Definitions

Unless otherwise indicated, all technical terms used herein have the same meaning as commonly understood by one of ordinary skill in the art to which this invention belongs. Unless otherwise indicated or obvious from context, the following terms have the following meanings:

The terms, “a,” “an,” and “the,” as used herein, include plural references unless the context clearly dictates otherwise.

The terms, “or” and “and/or,” as used herein, include any and all combinations of one or more of the associated listed items.

The terms, “including,” “includes,” “included,” and other forms, are not limiting.

The terms, “comprise” and its grammatical equivalents, as used herein, specify the presence of stated features, integers, steps, operations, elements, and/or components, but do not preclude the presence or addition of one or more other features, integers, steps, operations, elements, components, and/or groups thereof.

The term, “about,” as used herein in reference to a number or range of numbers, is understood to mean the stated number and numbers +/−10% thereof, or 10% below the lower listed limit and 10% above the higher listed limit for the values listed for a range.

The terms, “% identical,” “% identity,” “percent identity,” and grammatical equivalents thereof, as used herein, in the context of an amino acid sequence or nucleotide sequence, refer to the percent of residues that are identical between respective positions of two sequences when the two sequences are aligned for maximum sequence identity. The % identity is calculated by dividing the total number of the aligned residues by the number of the residues that are identical between the respective positions of the at least two sequences and multiplying by 100. Generally, computer programs can be employed for such calculations. Illustrative programs that compare and align pairs of sequences, include ALIGN (Myers and Miller, Comput Appl Biosci. 1988 March; 4(1):11-7), FASTA (Pearson and Lipman, Proc Natl Acad Sci USA. 1988 April; 85(8):2444-8; Pearson, Methods Enzymol. 1990; 183:63-98) and gapped BLAST (Altschul et al., Nucleic Acids Res. 1997 Sep. 1; 25(17):3389-40), BLASTP, BLASTN, or GCG (Devereux et al., Nucleic Acids Res. 1984 Jan. 11; 12(1 Pt 1):387-95).

The terms, “% complementary”, “% complementarity”, “percent complementary”, “percent complementarity” and grammatical equivalents thereof, as used interchangeably herein, in the context of two or more nucleic acid molecules, refer to the percent of nucleotides in two nucleotide sequences in said nucleic acid molecules of equal length that can undergo cumulative base pairing at two or more individual corresponding positions in an antiparallel orientation. Accordingly, the terms include nucleic acid sequences that are not completely complementary over their entire length, which indicates that the two or more nucleic acid molecules include one or more mismatches. A “mismatch” is present at any position in the two opposed nucleotides that are not complementary. The % complementary is calculated by dividing the total number of the complementary residues by the total number of the nucleotides in one of the equal length sequences, and multiplying by 100. Complete or total complementarity describes nucleotide sequences in 100% of the residues of a nucleotide sequence are complementary to residues in a reference nucleotide sequence. “Partially complementarity” describes nucleotide sequences in which at least 20%, but less than 100%, of the residues of a nucleotide sequence are complementary to residues in a reference nucleotide sequence. In some instances, at least 50%, but less than 100%, of the residues of a nucleotide sequence are complementary to residues in a reference nucleotide sequence. In some instances, at least 70%, 80%, 90% or 95%, but less than 100%, of the residues of a nucleotide sequence are complementary to residues in a reference nucleotide sequence. “Noncomplementary” describes nucleotide sequences in which less than 20% of the residues of a nucleotide sequence are complementary to residues in a reference nucleotide sequence.

The term, “% similarity,” as used herein, in the context of an amino acid sequence, refers to a value that is calculated by dividing a similarity score by the length of the alignment. The similarity of two amino acid sequences can be calculated by using a BLOSUM62 similarity matrix (Henikoff and Henikoff, Proc. Natl. Acad. Sci. USA., 89:10915-10919 (1992)) that is transformed so that any value ≥1 is replaced with +1 and any value ≤0 is replaced with 0. For example, an Ile (I) to Leu (L) substitution is scored at +2.0 by the BLOSUM62 similarity matrix, which in the transformed matrix is scored at +1. This transformation allows the calculation of percent similarity, rather than a similarity score. Alternately, when comparing two full protein sequences, the proteins can be aligned using pairwise MUSCLE alignment. Then, the % similarity can be scored at each residue and divided by the length of the alignment. For determining % similarity over a protein domain or motif, a multilevel consensus sequence (or PROSITE motif sequence) can be used to identify how strongly each domain or motif is conserved. In calculating the similarity of a domain or motif, the second and third levels of the multilevel sequence are treated as equivalent to the top level. Additionally, if a substitution could be treated as conservative with any of the amino acids in that position of the multilevel consensus sequence, +1 point is assigned. For example, given the multilevel consensus sequence: RLG and YCK, the test sequence QIQ would receive three points. This is because in the transformed BLOSUM62 matrix, each combination is scored as: Q-R: +1; Q-Y: +0; I-L: +1; I-C: +0; Q-G: +0; Q-K: +1 For each position, the highest score is used when calculating similarity. The % similarity can also be calculated using commercially available programs, such as the Geneious Prime software given the parameters matrix=BLOSUM62 and threshold ≥1.

The term “actuator,” as used herein in reference to a microfluidic device, refers to a component that causes a machine or other device to operate. An actuator may be a component of a machine that is responsible for moving and controlling a mechanism or system, such as, for example, controlling the opening or closing of a valve.

The terms, “amplification,” “amplifying,” and grammatical equivalents thereof, as used herein, refer to a process by which a nucleic acid molecule is enzymatically copied to generate a plurality of nucleic acid molecules containing the same sequence as the original nucleic acid molecule or a distinguishable portion thereof.

The terms, “bind,” “binding,” “interact” and “interacting,” as used herein, refer to a non-covalent interaction between macromolecules (e.g., between two polypeptides, between a polypeptide and a nucleic acid; between a polypeptide/guide nucleic acid complex and a target nucleic acid; and the like). While in a state of noncovalent interaction, the macromolecules are said to be “associated” or “interacting” or “binding” (e.g., when a molecule X is said to interact with a molecule Y, it is meant the molecule X binds to molecule Y in a non-covalent manner). Non-limiting examples of non-covalent interactions are ionic bonds, hydrogen bonds, van der Waals and hydrophobic interactions. Not all components of a binding interaction need be sequence-specific (e.g., contacts with phosphate residues in a DNA backbone), but some portions of a binding interaction may be sequence-specific.

The term, “base editor,” as used herein, refers to a fusion protein comprising a base editing enzyme fused to or linked to an effector protein. The base editing enzyme may be referred to as a fusion partner. The base editing enzyme can differ from a naturally occurring base editing enzyme. It is understood that any reference to a base editing enzyme herein also refers to a base editing enzyme variant. The base editor is functional when the effector protein is coupled to a guide nucleic acid. The guide nucleic acid imparts sequence specific activity to the base editor. By way of non-limiting example, the effector protein may comprise a catalytically inactive effector protein (e.g., a catalytically inactive variant of an effector protein described herein). Also, by way of non-limiting example, the base editing enzyme may comprise deaminase activity. Additional base editors are described herein.

The term, “catalytically inactive effector protein,” as used herein, refers to an effector protein that is modified relative to a naturally-occurring effector protein to have a reduced or eliminated catalytic activity relative to that of the naturally-occurring effector protein, but retains its ability to interact with a guide nucleic acid. The catalytic activity that is reduced or eliminated is often a nuclease activity. The naturally-occurring effector protein may be a wildtype protein. In some instances, the catalytically inactive effector protein is referred to as a catalytically inactive variant of an effector protein.

The term, “chamber,” and “channel,” as used herein, refer to a structural component of a microfluidic device, such as a separate section, area, or passageway, in which a composition, system, sample, fluid, gas, or loose material may be contained in isolation. Contained materials, such as a composition, system, sample, fluid, gas, or loose material, may be obstructed or allowed movement through a structural component in a controlled manner. Contained materials may be allowed movement from one structural component to another. In some instances, contained materials may be directed to interact with other non-structural components of a microfluidic device, such as one or more hydrogels, a well, a flow strip, a heating element, or combinations thereof. By way of non-limiting example, contained materials in a microfluidic device may be in fluid communication, optical communication, or thermal communication. Also, by way of non-limiting example, contained materials in a microfluidic device may be arranged in a sequence, in parallel, or both.

The term, “cis cleavage,” as used herein, refers to cleavage (hydrolysis of a phosphodiester bond) of a target nucleic acid by a complex of an effector protein and a guide nucleic acid (e.g., an RNP complex), wherein at least a portion of the guide nucleic acid is hybridized to at least a portion of the target nucleic acid. Cleavage may occur within or directly adjacent to the portion of the target nucleic acid that is hybridized to the portion of the guide nucleic acid.

The term, “codon optimized,” as used herein, refers to a mutation of a nucleotide sequence encoding a polypeptide, such as a nucleotide sequence encoding an effector protein, to mimic the codon preferences of the intended host organism or cell while encoding the same polypeptide. Thus, the codons can be changed, but the encoded polypeptide remains unchanged. For example, if the intended target cell was a human cell, a human codon-optimized nucleotide sequence encoding an effector protein could be used. As another non-limiting example, if the intended host cell were a mouse cell, then a mouse codon-optimized nucleotide sequence encoding an effector protein could be generated. As another non-limiting example, if the intended host cell were a eukaryotic cell, then a eukaryote codon-optimized nucleotide sequence encoding an effector protein could be generated. As another non-limiting example, if the intended host cell were a prokaryotic cell, then a prokaryote codon-optimized nucleotide sequence encoding an effector protein could be generated. Codon usage tables are readily available, for example, at the “Codon Usage Database” available at www.kazusa.or.jp/codon.

The terms, “complementary” and “complementarity,” as used herein, in the context of a nucleic acid molecule or nucleotide sequence, refer to the characteristic of a polynucleotide having nucleotides that can undergo cumulative base pairing with their Watson-Crick counterparts (C with G; or A with T) in a reference nucleic acid in antiparallel orientation. For example, when every nucleotide in a polynucleotide or a specified portion thereof forms a base pair with every nucleotide in an equal length sequence of a reference nucleic acid, that polynucleotide is said to be 100% complementary to the sequence of the reference nucleic acid. In a double stranded DNA or RNA sequence, the upper (sense) strand sequence is, in general, understood as going in the direction from its 5′- to 3′-end, and the complementary sequence is thus understood as the sequence of the lower (antisense) strand in the same direction as the upper strand. Following the same logic, the reverse sequence is understood as the sequence of the upper strand in the direction from its 3′- to its 5′-end, while the “reverse complement” sequence or the “reverse complementary” sequence is understood as the sequence of the lower strand in the direction of its 5′- to its 3′-end. Each nucleotide in a double stranded DNA or RNA molecule that is paired with its Watson-Crick counterpart can be referred to as its complementary nucleotide. The complementarity of modified or artificial base pairs can be based on other types of hydrogen bonding and/or hydrophobicity of bases and/or shape complementarity between bases.

The term, “cleavage assay,” as used herein, refers to an assay designed to visualize, quantitate or identify cleavage of a nucleic acid. In some instances, the cleavage activity may be cis cleavage activity. In some instances, the cleavage activity may be trans cleavage activity. A non-limiting example of a cis cleavage assay is provided in Example 2. A non-limiting example of a trans cleavage assay is provided in Example 3.

The terms, “cleave,” “cleaving” and “cleavage,” as used herein, in the context of a nucleic acid molecule or nuclease activity of an effector protein, refer to the hydrolysis of a phosphodiester bond of a nucleic acid molecule that results in breakage of that bond. The result of this breakage can be a nick (hydrolysis of a single phosphodiester bond on one side of a double-stranded molecule), single strand break (hydrolysis of a single phosphodiester bond on a single-stranded molecule) or double strand break (hydrolysis of two phosphodiester bonds on both sides of a double-stranded molecule) depending upon whether the nucleic acid molecule is single-stranded (e.g., ssDNA or ssRNA) or double-stranded (e.g., dsDNA) and the type of nuclease activity being catalyzed by the effector protein.

The term, “clustered regularly interspaced short palindromic repeats (CRISPR),” as used herein, refers to a segment of DNA found in the genomes of certain prokaryotic organisms, including some bacteria and archaea, that includes repeated short sequences of nucleotides interspersed at regular intervals between unique sequences of nucleotides derived from another organism.

The term, “conservative substitution,” as used herein, refers to the replacement of one amino acid for another such that the replacement takes place within a family of amino acids that are related in their side chains. Conversely, the term “non-conservative substitution” as used herein refers to the replacement of one amino acid residue for another that does not have a related side chain. Genetically encoded amino acids can be divided into four families having related side chains: (1) acidic (negatively charged): Asp (D), Glu (E); (2) basic (positively charged): Lys (K), Arg (R), His (H); (3) non-polar (hydrophobic): Cys (C), Ala (A), Val (V), Leu (L), Ile (I), Pro (P), Phe (F), Met (M), Trp (W), Gly (G), Tyr (Y), with non-polar also being subdivided into: (i) strongly hydrophobic: Ala (A), Val (V), Leu (L), Ile (I), Met (M), Phe (F); and (ii) moderately hydrophobic: Gly (G), Pro (P), Cys (C), Tyr (Y), Trp (W); and (4) uncharged polar: Asn (N), Gln (Q), Ser (S), Thr (T). Amino acids may be related by aliphatic side chains: Gly (G), Ala (A), Val (V), Leu (L), Ile (I), Ser (S), Thr (T), with Ser (S) and Thr (T) optionally being grouped separately as aliphatic-hydroxyl; Amino acids may be related by aromatic side chains: Phe (F), Tyr (Y), Trp (W). Amino acids may be related by amide side chains: Asn (N), Gln (Q). Amino acids may be related by sulfur-containing side chains: Cys (C) and Met (M).

The terms, “CRISPR RNA” and “crRNA,” as used herein, refer to a type of guide nucleic acid that is RNA comprising a first sequence that is capable of hybridizing to a target sequence of a target nucleic acid and a second sequence that is capable of interacting with an effector protein either directly (by being bound by an effector protein) or indirectly (e.g., by hybridization with a second nucleic acid molecule that can be bound by an effector). The first sequence and the second sequence are directly connected to each other or by a linker.

The term, “detection event,” as used herein in reference to a microfluidic device, generally refers to a moment in which compositions within the detection region of a microfluidic device exhibit binding of a programmable nuclease to a guide nucleic acid, binding of a guide nucleic acid to a target nucleic acid or target amplicon, and/or access to and cleavage of a reporter by an activated programmable nuclease, in accordance to the assay(s) being performed. A detection event may produce a detectable product or a detectable signal.

The term, “detectable product,” as used herein, refers to a unit produced after the cleavage of a reporter that is capable of being discovered, identified, perceived or noticed. A detectable product can comprise a detectable label and/or moiety that emits a detectable signal. A detectable product may include other components that are not capable of being readily discovered, identified, perceived or noticed at the same time as the detectable signal. For example, a detectable product may comprise remnants of the reporter. Accordingly, in some instances, the detectable product comprises RNA and/or DNA.

The term, “detectable signal,” as used herein, refers to an act, event, physical quantity or impulse that can be detected using optical, fluorescent, chemiluminescent, electrochemical or other detection methods known in the art.

The term, “detection region,” as used herein in reference to a microfluidic device, generally refers to a structural component which may comprise detection reagents that are immobilized, dried, or otherwise deposited thereto, including guide nucleic acids and/or reporters. A detection region may comprise one or more dried and/or immobilized amplification reagents including primers, polymerases, reverse transcriptase, and/or dNTPs. In some instances, a detection region may comprise a single detection array, one or more lateral flow strips, a detection tray, a capture antibody, or combinations thereof. Accordingly, in some instances, a detection region may comprise a plurality of microwells, detection chambers or channels, in fluid communication with amplification region(s). By way of a non-limiting example, a detection region may comprise three parallel detection chambers, each coupled to a single amplification region. One of ordinary skill in the art will recognize that the relative numbers of and relationships between amplification region(s) and detection region(s) may be varied depending on the assay(s) being performed. Also by way of a non-limiting example, compositions within the detection region of a microfluidic device may be agitated (e.g., via a spring-loaded valve piston) to facilitate binding of a programmable nuclease to a guide nucleic acid, binding of a guide nucleic acid to a target nucleic acid or target amplicon, and/or access to and cleavage of a reporter by an activated programmable nuclease.

The term, “donor nucleic acid,” as used herein, refers to a nucleic acid that is (designed or intended to be) incorporated into a target nucleic acid or target sequence.

The term, “effector protein,” as used herein, refers to a protein, polypeptide, or peptide that is capable of interacting with a nucleic acid, such as a guide nucleic acid, to form a complex (e.g., a RNP complex), wherein the complex interacts with a target nucleic acid.

The terms, “effector partner” and “partner polypeptide” as used herein, refer to a polypeptide that does not have 100% sequence identity with an effector protein described herein. In some instances, an effector partner described herein may be found in a homologous genome as an effector protein described herein.

The term, “engineered modification,” as used herein, refers to a structural change of one or more nucleic acid residues of a nucleotide sequence or one or more amino acid residue of an amino acid sequence, such as chemical modification of one or more nucleobases; or a chemical change to the phosphate backbone, a nucleotide, a nucleobase, or a nucleoside. Such modifications can be made to an effector protein amino acid sequence or guide nucleic acid nucleotide sequence, or any sequence disclosed herein (e.g., a nucleic acid encoding an effector protein or a nucleic acid that encodes a guide nucleic acid). Methods of modifying a nucleic acid or amino acid sequence are known. One of ordinary skill in the art will appreciate that the engineered modification(s) may be located at any position(s) of a nucleic acid such that the function of the nucleic acid, protein, composition or system is not substantially decreased. Nucleic acids provided herein can be prepared according to any available technique including, but not limited to chemical synthesis, enzymatic synthesis, which is generally termed in vitro-transcription, cloning, enzymatic, or chemical cleavage, etc. In some instances, the nucleic acids provided herein are not uniformly modified along the entire length of the molecule. Different nucleotide modifications and/or backbone structures can exist at various positions within the nucleic acid.

The term, “functional domain,” as used herein, refers to a region of one or more amino acids in a protein that is required for an activity of the protein, or the full extent of that activity, as measured in an in vitro assay. Activities include, but are not limited to nucleic acid binding, nucleic acid editing, nucleic acid modifying, nucleic acid cleaving, protein binding. The absence of the functional domain, including mutations of the functional domain, would abolish or reduce activity.

The term, “functional fragment,” as used herein, refers to a fragment of a protein that retains some function relative to the entire protein. Non-limiting examples of functions are nucleic acid binding, nucleic acid editing, protein binding, nuclease activity, nickase activity, deaminase activity, demethylase activity, or acetylation activity. A functional fragment may be a recognized functional domain, e.g., a catalytic domain such as, but not limited to, a HEPN domain.

The term, “functional protein,” as used herein, refers to protein that retains at least some if not all activity relative to the wildtype protein. A functional protein can also include a protein having enhanced activity relative to the wildtype protein. Assays are known and available for detecting and quantifying protein activity, e.g., colorimetric and fluorescent assays. In some instances, a functional protein is a wildtype protein. In some instances, a functional protein is a functional portion of a wildtype protein.

The term, “fused,” as used herein, refers to at least two sequences that are connected together, such as by a linker, or by conjugation (e.g., chemical conjugation or enzymatic conjugation). The term “fused” includes a linker.

The term, “fusion protein,” as used herein, refers to a protein comprising at least two heterologous polypeptides. The fusion protein may comprise one or more effector protein and fusion partner. In some instances, an effector protein and fusion partner are not found connected to one another as a native protein or complex that occurs together in nature.

The term, “fusion partner,” as used herein, refers to a protein, polypeptide or peptide that is fused, or linked by a linker, to one or more effector protein. The fusion partner can impart some function to the fusion protein that is not provided by the effector protein.

The term, “genetic disease,” as used herein, refers to a disease, disorder, condition, or syndrome associated with or caused by one or more mutations in the DNA of an organism having the genetic disease.

The term, “guide nucleic acid,” as used herein, refers to a nucleic acid that, when in a complex with one or more polypeptides described herein (e.g., an RNP complex) can impart sequence selectivity to the complex when the complex interacts with a target nucleic acid. A guide nucleic acid may be referred to interchangeably as a guide RNA, however it is understood that guide nucleic acids may comprise deoxyribonucleotides (DNA), ribonucleotides (RNA), a combination thereof (e.g., RNA with a thymine base), biochemically or chemically modified nucleobases (e.g., one or more engineered modifications described herein), or combinations thereof.

The terms “heater”, “heating unit”, “heating element”, “heat source”, and the like, as used herein in reference to a device, generally refers to an element that is configured to produce heat and is in thermal communication with a portion of a device.

The term, “heterologous,” as used herein, refers to at least two different polypeptide sequences that are not found similarly connected to one another in a native nucleic acid or protein. A protein that is heterologous to the effector protein is a protein that is not covalently linked by an amide bond to the effector protein in nature. In some instances, a heterologous protein is not encoded by a species that encodes the effector protein. A guide nucleic acid may comprise “heterologous” sequences, which means that it includes a first sequence and a second sequence, wherein the first sequence and the second sequence are not found covalently linked by a phosphodiester bond in nature. Thus, the first sequence is considered to be heterologous with the second sequence, and the guide nucleic acid may be referred to as a heterologous guide nucleic acid.

The terms, “hybridize,” “hybridizable” and grammatical equivalents thereof, refer to a nucleotide sequence that is able to noncovalently interact, i.e. form Watson-Crick base pairs and/or G/U base pairs, or anneal, to another nucleotide sequence in a sequence-specific, antiparallel, manner (i.e., a nucleotide sequence specifically interacts to a complementary nucleotide sequence) under the appropriate in vitro and/or in vivo conditions of temperature and solution ionic strength. Standard Watson-Crick base-pairing includes: adenine (A) pairing with thymidine (T), adenine (A) pairing with uracil (U), and guanine (G) pairing with cytosine (C) for both DNA and RNA. In addition, for hybridization between two RNA molecules (e.g., dsRNA), and for hybridization of a DNA molecule with an RNA molecule (e.g., when a DNA target nucleic acid base pairs with a guide RNA, etc.): guanine (G) can also base pair with uracil (U). For example, G/U base-pairing is at least partially responsible for the degeneracy (i.e., redundancy) of the genetic code in the context of tRNA anti-codon base-pairing with codons in mRNA. Thus, a guanine (G) can be considered complementary to both an uracil (U) and to an adenine (A). Accordingly, when a G/U base-pair can be made at a given nucleotide position, the position is not considered to be noncomplementary, but is instead considered to be complementary. While hybridization typically occurs between two nucleotide sequences that are complementary, mismatches between bases are possible. It is understood that two nucleotide sequences need not be 100% complementary to be specifically hybridizable, hybridizable, partially hybridizable, or for hybridization to occur. Moreover, a nucleotide sequence may hybridize over one or more segments such that intervening or adjacent segments are not involved in the hybridization event (e.g., a bulge, a loop structure or hairpin structure, etc.). The conditions appropriate for hybridization between two nucleotide sequences depend on the length of the sequence and the degree of complementarity, variables which are well known in the art. For hybridizations between nucleic acids with short stretches of complementarity (e.g. complementarity over 35 or less, 30 or less, 25 or less, 22 or less, 20 or less, or 18 or less nucleotides) the position of mismatches may become important (see Sambrook et al., supra, 11.7-11.8). Typically, the length for a hybridizable nucleic acid is 8 nucleotides or more (e.g., 10 nucleotides or more, 12 nucleotides or more, 15 nucleotides or more, 20 nucleotides or more, 22 nucleotides or more, 25 nucleotides or more, or 30 nucleotides or more). Any suitable in vitro assay may be utilized to assess whether two sequences “hybridize”. One such assay is a melting point analysis where the greater the degree of complementarity between two nucleotide sequences, the greater the value of the melting temperature (Tm) for hybrids of nucleic acids having those sequences. The conditions of temperature and ionic strength determine the “stringency” of the hybridization. Temperature, wash solution salt concentration, and other conditions may be adjusted as necessary according to factors such as length of the region of complementation and the degree of complementation. Hybridization and washing conditions are well known and exemplified in Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein; and Sambrook, J. and Russell, W., Molecular Cloning: A Laboratory Manual, Third Edition, Cold Spring Harbor Laboratory Press, Cold Spring Harbor (2001).

The term, “indel,” as used herein, refers to an insertion-deletion or indel mutation, which is a type of genetic mutation that results from the insertion and/or deletion of one or more nucleotide in a target nucleic acid. An indel can vary in length (e.g., 1 to 1,000 nucleotides in length) and be detected by any suitable method, including sequencing.

The term, “indel percentage,” as used herein, refers to a percentage of sequencing reads that show at least one nucleotide has been edited from the insertion and/or deletion of nucleotides regardless of the size of insertion or deletion, or number of nucleotides edited. For example, if there is at least one nucleotide deletion detected in a given target nucleic acid, it counts towards the percent indel value. As another example, if one copy of the target nucleic acid has one nucleotide deleted, and another copy of the target nucleic acid has 10 nucleotides deleted, they are counted the same. This number reflects the percentage of target nucleic acids that are edited by a given effector protein.

The term, “in vitro,” as used herein, refers to describing something outside an organism. An in vitro system, composition or method may take place in a container for holding laboratory reagents such that it is separated from the biological source from which a material in the container is obtained. In vitro assays can encompass cell-based assays in which living or dead cells are employed. In vitro assays can also encompass a cell-free assay in which no intact cells are employed. The term “in vivo” is used to describe an event that takes place within an organism. The term “ex vivo” is used to describe an event that takes place in a cell that has been obtained from an organism. An ex vivo assay is not performed on a subject. Rather, it is performed upon a sample separate from a subject.

The terms, “length” and “linked nucleosides,” as used herein, refer to a nucleic acid (polynucleotide) or polypeptide, may be expressed as “kilobases” (kb) or “base pairs (bp),”. Thus, a length of 1 kb refers to a length of 1000 linked nucleosides, and a length of 500 bp refers to a length of 500 linked nucleosides. Similarly, a protein having a length of 500 linked amino acids may also be simply described as having a length of 500 amino acids.

The term, “linker,” as used herein, refers to a covalent bond or molecule that links a first polypeptide to a second polypeptide (e.g., by an amide bond) or a first nucleic acid to a second nucleic acid (e.g., by a phosphodiester bond).

The term, “modified target nucleic acid,” as used herein, refers to a target nucleic acid that has undergone a physical alteration from its original form, for example, after contact with an effector protein. In some instances, the modifying is an alteration in the sequence of the target nucleic acid. In some instances, the modified target nucleic acid comprises an insertion, deletion, or substitution of one or more nucleotides compared to the unmodified target nucleic acid.

The term, “mutation,” as used herein, refers to an alteration that changes an amino acid residue or a nucleotide as described herein. Such an alteration can include, for example, deletions, insertions, and/or substitutions. The mutation can refer to a change in structure of an amino acid residue or nucleotide relative to the starting or reference residue or nucleotide. A mutation of an amino acid residue includes, for example, deletions, insertions and substituting one amino acid residue for a structurally different amino acid residue. Such substitutions can be a conservative substitution, a non-conservative substitution, a substitution to a specific sub-class of amino acids, or a combination thereof as described herein. A mutation of a nucleotide includes, for example, changing one naturally occurring base for a different naturally occurring base, such as changing an adenine to a thymine or a guanine to a cytosine or an adenine to a cytosine or a guanine to a thymine. A mutation of a nucleotide base may result in a structural and/or functional alteration of the encoding peptide, polypeptide or protein by changing the encoded amino acid residue of the peptide, polypeptide or protein. A mutation of a nucleotide base may not result in an alteration of the amino acid sequence or function of encoded peptide, polypeptide or protein, also known as a silent mutation. Methods of mutating an amino acid residue or a nucleotide are well known.

The terms, “mutation associated with a disease” and “mutation associated with a genetic disorder,” as used herein, refer to the co-occurrence of a mutation and the phenotype of a disease. The mutation may occur in a gene, wherein transcription or translation products from the gene occur at a significantly abnormal level or in an abnormal form in a cell or subject harboring the mutation as compared to a non-disease control subject not having the mutation.

The term, “nickase,” as used herein, refers to an enzyme that possess catalytic activity for single stranded nucleic acid cleavage of a double stranded nucleic acid.

The term, “nickase activity,” as used herein, refers to catalytic activity that results in single stranded nucleic acid cleavage of a double stranded nucleic acid.

The terms, “non-naturally occurring” and “engineered,” as used herein, refer to indicate involvement of the hand of man. The terms, when referring to a nucleic acid, nucleotide, protein, polypeptide, peptide or amino acid, refer to a molecule, such as but not limited to, a nucleic acid, nucleotide, protein, polypeptide, peptide or amino acid refers to a modification of that molecule (e.g., chemical modification, nucleotide sequence, or amino acid sequence) that is not present in the naturally molecule. The terms, when referring to a composition or system described herein, refer to a composition or system having at least one component that is not naturally associated with the other components of the composition or system. By way of a non-limiting example, a composition may include an effector protein and a guide nucleic acid that do not naturally occur together. Conversely, and as a non-limiting further clarifying example, an effector protein or guide nucleic acid that is “natural,” “naturally-occurring,” or “found in nature” includes an effector protein and a guide nucleic acid from a cell or organism that have not been genetically modified by the hand of man.

The terms, “nuclease” and “endonuclease” as used herein, refer to an enzyme which possesses catalytic activity for nucleic acid cleavage.

The term, “nuclease activity,” as used herein, refers to catalytic activity that results in nucleic acid cleavage (e.g., ribonuclease activity (ribonucleic acid cleavage), or deoxyribonuclease activity (deoxyribonucleic acid cleavage), etc.).

The term, “nucleic acid,” as used herein, refers to a polymer of nucleotides. A nucleic acid may comprise ribonucleotides, deoxyribonucleotides, combinations thereof, and modified versions of the same. A nucleic acid may be single-stranded or double-stranded, unless specified. Non-limiting examples of nucleic acids are double stranded DNA (dsDNA), single stranded (ssDNA), messenger RNA, genomic DNA, cDNA, DNA-RNA hybrids, and a polymer comprising purine and pyrimidine bases or other natural, chemically or biochemically modified, non-natural, or derivatized nucleotide bases. Accordingly, nucleic acids as described herein may comprise one or more mutations, one or more engineered modifications, or both.

The term, “nucleic acid expression vector,” as used herein, refers to a plasmid that can be used to express a nucleic acid of interest.

The term, “nuclear localization signal (NLS),” as used herein, refers to an entity (e.g., peptide) that facilitates localization of a nucleic acid, protein, or small molecule to the nucleus, when present in a cell that contains a nuclear compartment.

The terms, “nucleotide(s)” and “nucleoside(s)”, as used herein, in the context of a nucleic acid molecule having multiple residues, refer to describing the sugar and base of the residue contained in the nucleic acid molecule. Similarly, a skilled artisan could understand that linked nucleotides and/or linked nucleosides, as used in the context of a nucleic acid having multiple linked residues, are interchangeable and describe linked sugars and bases of residues contained in a nucleic acid molecule. When referring to a “nucleobase(s)”, or linked nucleobase, as used in the context of a nucleic acid molecule, it can be understood as describing the base of the residue contained in the nucleic acid molecule, for example, the base of a nucleotide, nucleosides, or linked nucleotides or linked nucleosides. A person of ordinary skill in the art when referring to nucleotides, nucleosides, and/or nucleobases would also understand the differences between RNA and DNA (generally the exchange of uridine for thymidine or vice versa) and the presence of nucleoside analogs, such as modified uridines, do not contribute to differences in identity or complementarity among polynucleotides as long as the relevant nucleotides (such as thymidine, uridine, or modified uridine) have the same complement (e.g., adenosine for all of thymidine, uridine, or modified uridine; another example is cytosine and 5-methylcytosine, both of which have guanosine or modified guanosine as a complement). Thus, for example, the sequence 5′-AXG where X is any modified uridine, such as pseudouridine, NI-methyl pseudouridine, or 5-methoxyuridine, is considered 100% identical to AUG in that both are perfectly complementary to the same sequence (5′-CAU).

The term, “pharmaceutically acceptable excipient, carrier or diluent,” as used herein, refers to any substance formulated alongside the active ingredient of a pharmaceutical composition that allows the active ingredient to retain biological activity and is non-reactive with the subject's immune system. Such a substance can be included for the purpose of long-term stabilization, bulking up solid formulations that contain potent active ingredients in small amounts, or to confer a therapeutic enhancement on the active ingredient in the final dosage form, such as facilitating absorption, reducing viscosity, or enhancing solubility. The selection of appropriate substance can depend upon the route of administration and the dosage form, as well as the active ingredient and other factors. Compositions having such substances can be formulated by suitable methods (see, e.g., Remington's Pharmaceutical Sciences, 18th edition, A. Gennaro, ed., Mack Publishing Co., Easton, Pa., 1990; and Remington, The Science and Practice of Pharmacy 21st Ed. Mack Publishing, 2005).

The terms, “polypeptide” and “protein,” as used herein, refer to a polymeric form of amino acids. A polypeptide may include coded and non-coded amino acids, chemically or biochemically modified or derivatized amino acids, and polypeptides having modified peptide backbones. Accordingly, polypeptides as described herein may comprise one or more mutations, one or more engineered modifications, or both. It is understood that when describing coding sequences of polypeptides described herein, said coding sequences do not necessarily require a codon encoding an N-terminal Methionine (M) or a Valine (V) as described for the effector proteins described herein. One skilled in the art would understand that a start codon could be replaced or substituted with a start codon that encodes for an amino acid residue sufficient for initiating translation in a host cell. In some instances, when a heterologous peptide, such as a fusion partner protein, protein tag or NLS, is located at the N terminus of the effector protein, a start codon for the heterologous peptide serves as a start codon for the effector protein as well. Thus, the natural start codon encoding an amino acid residue sufficient for initiating translation (e.g., Methionine (M) or a Valine (V)) of the effector protein may be removed or absent.

The terms, “promoter” and “promoter sequence,” as used herein, refer to a DNA regulatory region capable of binding RNA polymerase and initiating transcription of a downstream (3′ direction) coding or non-coding sequence. A transcription initiation site, as well as protein binding domains responsible for the binding of RNA polymerase, can also be found in a promoter region. Eukaryotic promoters will often, but not always, contain “TATA” boxes and “CAT” boxes. Various promoters, including inducible promoters, may be used to drive expression by the various vectors of the present disclosure.

The terms, “protospacer adjacent motif” and “PAM,” as used herein, refer to a nucleotide sequence found in a target nucleic acid that directs an effector protein to edit the target nucleic acid at a specific location. In some instances, a PAM is required for a complex of an effector protein and a guide nucleic acid (e.g., an RNP complex) to hybridize to and edit the target nucleic acid. In some instances, the complex does not require a PAM to edit the target nucleic acid.

The term “reagent mix”, “reagent master mix”, “reagents”, and the like, as used herein, generally refers to a formulation comprising one or more chemicals that partake in a reaction that the formulation is intended for.

The term, “recombinant,” as used herein, in the context of proteins, polypeptides, peptides and nucleic acids, refers to proteins, polypeptides, peptides and nucleic acids that are products of various combinations of cloning, restriction, and/or ligation steps resulting in a construct having a structural coding or non-coding sequence distinguishable from endogenous nucleic acids found in natural systems.

The term, “regulatory element,” used herein, refers to transcriptional and translational control sequences, such as promoters, enhancers, polyadenylation signals, terminators, protein degradation signals, and the like, that provide for and/or regulate transcription of a non-coding sequence (e.g., a guide nucleic acid) or a coding sequence (e.g., effector proteins, fusion proteins, and the like) and/or regulate translation of an encoded polypeptide.

The term, “repeat sequence,” as used herein, refers to a sequence of nucleotides in a guide nucleic acid that is capable of, at least partially, interacting with an effector protein.

The terms, “reporter,” “reporter nucleic acid,” and “reporter molecule,” as used herein, are used interchangeably and refer to a non-target nucleic acid molecule that can provide a detectable signal upon cleavage by an effector protein. Examples of detectable signals and detectable moieties that generate detectable signals are provided herein.

The term, “sample,” as used herein, refers to something comprising a target nucleic acid. In some instances, the sample is a biological sample, such as a biological fluid or tissue sample. In some instances, the sample is an environmental sample. The sample may be a biological sample or environmental sample that is modified or manipulated. By way of non-limiting example, samples may be modified or manipulated with purification techniques, heat, nucleic acid amplification, salts and buffers.

The terms “sample interface” and “sample input,” as used herein in reference to a microfluidic device, generally refer to a structural component capable of receiving a composition comprising a target nucleic acid as disclosed herein (e.g., a sample). The composition comprising a target nucleic acid may be a sample as defined above, which may be collected with a sample collector (e.g., swab, tube, etc.) before being received in a sample interface. By way of a non-limiting example, the sample may be directly collected at the sample interface (e.g., without the use of a separate sample collector). In some instances, a sample interface may be in fluid communication with a plurality of chambers, channels, or reservoirs of a microfluidic device. In some instances, the sample interface is fluidically connected to the plurality of chambers via lysis, preparation, amplification, or detection regions.

The term, “single nucleic acid system,” as used herein, refers to a system that uses a guide nucleic acid complexed with one or more polypeptides described herein, wherein the complex is capable of interacting with a target nucleic acid in a sequence specific manner, and wherein the guide nucleic acid is capable of non-covalently interacting with the one or more polypeptides described herein, and wherein the guide nucleic acid is capable of hybridizing with a target sequence of the target nucleic acid. A single nucleic acid system lacks a duplex of a guide nucleic acid as hybridized to a second nucleic acid, wherein in such a duplex the second nucleic acid, and not the guide nucleic acid, is capable of interacting with the effector protein.

The term, “spacer sequence,” as used herein, refers to a nucleotide sequence in a guide nucleic acid that is capable of, at least partially, hybridizing to an equal length portion of a sequence (e.g., a target sequence) of a target nucleic acid.

The term, “subject,” as used herein, refers to an animal. The subject may be a mammal. The subject may be a human. The subject may be diagnosed or at risk for a disease.

The term, “syndrome,” as used herein, refers to a group of symptoms which, taken together, characterize a condition.

The term, “target nucleic acid,” as used herein, refers to a nucleic acid that is selected as the nucleic acid for editing, modifying, binding, hybridization or any other activity of or interaction with a nucleic acid, protein, polypeptide, or peptide described herein. A target nucleic acid may comprise RNA, DNA, or a combination thereof. A target nucleic acid may be single-stranded (e.g., single-stranded RNA or single-stranded DNA) or double-stranded (e.g., double-stranded DNA).

The term, “target sequence,” as used herein, in the context of a target nucleic acid, refers to a nucleotide sequence found within a target nucleic acid. Such a nucleotide sequence can, for example, hybridize to a respective length portion of a guide nucleic acid.

The term, “trans cleavage,” as used herein, in the context of cleavage (e.g., hydrolysis of a phosphodiester bond) of one or more target nucleic acids or non-target nucleic acids, or both, by an effector protein that is complexed with a guide nucleic acid and the target nucleic acid. Trans cleavage activity may be triggered by the hybridization of a guide nucleic acid to a target nucleic acid. The effector may cleave a target strand as well as non-target strand, wherein the target nucleic is a double stranded nucleic acid. Trans cleavage of the target nucleic acid may occur away from (e.g., not within or directly adjacent to) the portion of the target nucleic acid that is hybridized to the portion of the guide nucleic acid.

The term, “transgene,” as used herein, refers to a nucleotide sequence that is inserted into a cell for expression of said nucleotide sequence in the cell. A transgene is meant to include (1) a nucleotide sequence that is not naturally found in the cell (e.g., a heterologous nucleotide sequence); (2) a nucleotide sequence that is a mutant form of a nucleotide sequence naturally found in the cell into which it has been introduced; (3) a nucleotide sequence that serves to add additional copies of the same (e.g., exogenous or homologous) or a similar nucleotide sequence naturally occurring in the cell into which it has been introduced; or (4) a silent naturally occurring or homologous nucleotide sequence whose expression is induced in the cell into which it has been introduced. A donor nucleic acid can comprise a transgene. The cell in which transgene expression occurs can be a target cell, such as a host cell.

The term, “transposase activity,” as used herein, refers to catalytic activity that results in the transposition of a first nucleic acid into a second nucleic acid.

The terms, “treatment” and “treating,” as used herein, refer to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying, or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The term, “valve,” as used herein, refers to a mechanism or device for directing, regulating, controlling, or obstructing the passage of fluid, gas, or loose materials through an opening or passageway. A valve may regulate the movement of fluid through an opening in one direction only. A valve may operate automatically, pneumatically, hydraulically, mechanically, electrically, chemically or combinations thereof.

The term, “variant,” as used herein, refers to a form or version of a protein that differs from the wild-type protein. A variant may have a different function or activity relative to the wild-type protein.

The term, “viral vector,” as used herein, refers to a nucleic acid to be delivered into a host cell by a recombinantly produced virus or viral particle.

### III. Introduction

Disclosed herein are compositions, systems, devices, kits, and methods comprising at least one of:
a) a polypeptide or a nucleic acid encoding the polypeptide; and
b) a guide nucleic acid or a nucleic acid encoding the guide nucleic acid.

Polypeptides described herein may bind and, optionally, cleave nucleic acids in a sequence-specific manner. Polypeptides described herein may also cleave the target nucleic acid within a target sequence or at a position adjacent to the target sequence. In some embodiments, a polypeptide is activated when it binds a certain sequence of a nucleic acid described herein, allowing the polypeptide to cleave a region of a target nucleic acid that is near, but not adjacent to the target sequence. A polypeptide may be an effector protein, such as a CRISPR-associated (Cas) protein, which may bind a guide nucleic acid that imparts activity or sequence selectivity to the polypeptide. An effector protein may also be referred to as a programmable nuclease because the nuclease activity of the protein may be directed to different target nucleic acids by way of revising the guide nucleic acid that the protein binds.

In some embodiments, compositions, systems, devices, kits, and methods comprising effector proteins and guide nucleic acids comprise a first region or sequence, at least a portion of which interacts with a polypeptide. In some embodiments, the first region or sequence comprises a sequence that is similar or identical to a repeat sequence. In some embodiments, compositions, systems, devices, kits, and methods comprising effector proteins and guide nucleic acids comprise a second region or sequence that is at least partially complementary to a target sequence of a target nucleic acid, and which, in some embodiments, is referred to as a spacer sequence. In some embodiments, compositions, systems, devices, kits, and methods comprising effector proteins and guide nucleic acids comprise a first region or sequence and a second region or sequence, wherein the first region or sequence and the second region or sequence are heterologous to each other. In some embodiments, the first region or sequence is covalently linked to the 5′ end of the second region or sequence. In some embodiments, the first region or sequence at least partially interacts with the polypeptide. In some embodiments, compositions, systems, devices, kits, and methods described herein comprise a guide nucleic acid, wherein the guide nucleic acid comprises a crRNA.

In some embodiments, effector proteins disclosed herein binds and/or cleaves nucleic acids such as RNA, single stranded RNA, double stranded RNA, linear single-stranded RNA, circular RNA, coding RNA, non-coding RNA, or combinations thereof. In some embodiments, polypeptides disclosed herein provide binding activity, cis cleavage activity, trans cleavage activity, nickase activity, nuclease activity, or a combination thereof.

The compositions, systems, devices, kits, and methods described herein are non-naturally occurring. In some embodiments, compositions, systems, devices, kits, and methods comprise an engineered guide nucleic acid (also referred to herein as a guide nucleic acid) or a use thereof. In some embodiments, compositions, systems, devices, kits, and methods comprise an engineered protein or a use thereof. In some embodiments, compositions, systems, devices, kits, and methods comprise an isolated polypeptide or a use thereof. In general, compositions, systems, devices, kits, and methods described herein are not found in nature. In some embodiments, compositions, systems, devices, kits, and methods described herein comprise at least one non-naturally occurring component. For example, in some embodiments, disclosed compositions, systems, devices, kits, and methods comprise a guide nucleic acid, wherein the nucleotide sequence of the guide nucleic acid is different or modified from that of a naturally-occurring guide nucleic acid.

In some embodiments, compositions, systems, devices, kits, and methods comprise at least two components that do not naturally occur together. For example, in some embodiments, disclosed compositions, systems, devices, kits, and methods comprise a guide nucleic acid comprising a first region or sequence, at least a portion of which, interacts with a polypeptide, and a second region or sequence that is at least partially complementary to a target sequence in a target nucleic acid, wherein the first region or sequence and second region or sequence do not naturally occur together and/or are heterologous to each other. Also, by way of non-limiting example, in some embodiments, disclosed compositions, systems, and methods comprise a guide nucleic acid and an effector protein that do not naturally occur together. Likewise, by way of non-limiting example, disclosed compositions, systems, and methods comprise a ribonucleotide-protein (RNP) complex comprising an effector protein and a guide nucleic acid that do not occur together in nature. Conversely, and for clarity, an effector protein or guide nucleic acid that is “natural,” “naturally-occurring,” or “found in nature” includes effector proteins and guide nucleic acids from cells or organisms that have not been genetically modified by a human or machine.

In some embodiments, the guide nucleic acid comprises a non-natural nucleotide sequence. In some embodiments, the non-natural nucleotide sequence is a nucleotide sequence that is not found in nature. In some embodiments, the non-natural nucleotide sequence comprises a portion of a naturally-occurring nucleotide sequence, wherein the portion of the naturally-occurring nucleotide sequence is not present in nature absent the remainder of the naturally-occurring nucleotide sequence. In some embodiments, the guide nucleic acid comprises two naturally-occurring nucleotide sequences arranged in an order or proximity that is not observed in nature. In some embodiments, compositions and systems comprise a ribonucleotide complex comprising an effector protein and a guide nucleic acid that do not occur together in nature. In some embodiments, guide nucleic acids comprise a first region or sequence and a second region or sequence that do not occur naturally together. For example, in some embodiments, a guide nucleic acid comprises a naturally-occurring repeat sequence and a spacer sequence that is complementary to a naturally-occurring eukaryotic nucleotide sequence. In some embodiments, the guide nucleic acid comprises a repeat sequence that occurs naturally in an organism and a spacer sequence that does not occur naturally in that organism. In some embodiments, a guide nucleic acid comprises a first region or sequence that occurs in a first organism and a second region or sequence that occurs in a second organism, wherein the first organism and the second organism are different. In some embodiments, the guide nucleic acid comprises a third region or sequence disposed at a 3′ or 5′ end of the guide nucleic acid, or between the first and second regions or sequences of the guide nucleic acid. In some embodiments, the guide nucleic acid comprises two heterologous nucleotide sequences arranged in an order or proximity that is not observed in nature. Therefore, compositions and systems described herein are not naturally occurring.

In some embodiments, compositions, systems, devices, kits, and methods described herein comprise a polypeptide (e.g., an effector protein, a fusion partner, a fusion protein, or a combination thereof) that is similar to a naturally occurring polypeptide. In some embodiments, the polypeptide lacks a portion of the naturally occurring polypeptide. In some embodiments, the polypeptide comprises a mutation relative to the naturally-occurring polypeptide, wherein the mutation is not found in nature. In some embodiments, the polypeptide also comprises at least one additional amino acid relative to the naturally-occurring polypeptide. In some embodiments, the polypeptide comprises a heterologous polypeptide. For example, in some embodiments, the polypeptide comprises an addition of a nuclear localization signal relative to the natural occurring polypeptide. In some embodiments, a nucleotide sequence encoding the polypeptide is codon optimized (e.g., for expression in a eukaryotic cell) relative to the naturally occurring sequence.

### IV. Polypeptide Systems

Provided herein are compositions, systems and methods comprising a polypeptide or polypeptide system, wherein the polypeptide or polypeptide system described herein comprises one or more effector proteins or variants thereof, one or more effector partners or variants thereof, one or more linkers for peptides, or combinations thereof. A polypeptide as described herein can also be referred to as a protein in the present disclosure.

### Effector Proteins

Provided herein are compositions, systems, devices, kits, and methods comprising an effector protein or a use thereof.

An effector protein provided herein interacts with a guide nucleic acid, or a portion thereof, to form a complex. In some embodiments, the complex interacts with a target nucleic acid, a non-target nucleic acid, or both. In some embodiments, an interaction between the complex and a target nucleic acid, a non-target nucleic acid, or both comprises hybridization of the guide nucleic acid to the target nucleic acid, modification of the target nucleic acid and/or the non-target nucleic acid by the effector protein, or combinations thereof. In some embodiments, the effector protein complexed with a guide nucleic acid hybridizes to the target nucleic acid (e.g., RNA). In some embodiments, the target RNA is double-stranded RNA, single-stranded RNA, linear single-stranded RNA, circular RNA, or combinations thereof. In some embodiments, the effector protein cleaves the non-target nucleic acid (e.g., RNA, DNA, or combinations thereof). In some embodiments, the effector protein hybridizes to the target nucleic acid (e.g., RNA), and cleaves the non-target nucleic acid (e.g., ssDNA, non-target RNA).

In some embodiments, modification activity of an effector protein results in: cleavage of a target nucleic acid, deletion of one or more nucleotides of a target nucleic acid, insertion of one or more nucleotides into a target nucleic acid, substitution of one or more nucleotides of a target nucleic acid with an alternative nucleotide, more than one of the foregoing, or any combination thereof. In some embodiments, modification activity of an effector protein results in certain measurable features, such as alternative splicing of a target nucleic acid (e.g., removal, degradation, and/or rejoining of a sequence of interest comprised in a target nucleic acid). In some embodiments, an ability of an effector protein to modify a target nucleic acid depends upon the effector protein being complexed with a guide nucleic acid, the guide nucleic acid being hybridized to a target sequence of the target nucleic acid, or combinations thereof.

The modification of the target nucleic acid generated by an effector protein, as a non-limiting example, results in modulation of the expression of the target nucleic acid (e.g., increasing or decreasing expression of the nucleic acid) or modulation of the activity of a translation product of the target nucleic acid (e.g., inactivation of a protein binding to an RNA molecule or hybridization). Accordingly, in some embodiments, provided herein are methods of modifying a target nucleic acid using an effector protein of the present disclosure, or compositions or systems thereof. Also provided herein are methods of modulating expression of a target nucleic acid using an effector protein of the present disclosure, or compositions or systems thereof. Further provided herein are methods of modulating the activity of a translation product of a target nucleic acid using an effector protein of the present disclosure, or compositions or systems thereof.

In some embodiments, effector proteins disclosed herein provide cleavage activity, such as cis cleavage activity, trans cleavage activity, nickase activity, nuclease activity, other activity, or a combination thereof. In general, effector proteins described herein modify a target nucleic acid by cis cleavage activity on the target nucleic acid. Alternatively or additionally, effector proteins described herein modify a non-target nucleic acid by trans cleavage activity on the non-target nucleic acid. In some embodiments, effector proteins disclosed herein comprise a HEPN domain capable of cleavage activity. A HEPN domain includes a region of an effector protein that is capable of cleaving a target nucleic acid (e.g., RNA), and in certain instances, of processing pre-crRNA. In some embodiments, the HEPN domain is located near the C terminus of the effector proteins described herein. In some embodiments, a HEPN domain comprises a R-X₄-H motif capable of cleaving a target nucleic acid (e.g., RNA), which, in some embodiments, results in RNA degradation (e.g., self-regulated RNase activity, pre-crRNA processing). In some embodiments, an effector protein comprises two HEPN domains (e.g., HEPN-1 and HEPN-2). In some embodiments, effector proteins disclosed herein comprise two HEPN domains or dual HEPN domains capable of cleavage activity. In some embodiments, effector proteins disclosed herein cleave nucleic acids having a RNA sequence, including single stranded RNA (ssRNA) and double stranded RNA (dsRNA). In some embodiments, effector proteins disclosed herein cleave the target nucleic acid within the target sequence or within 50 nucleotides of the 5′ or 3′ end of the target sequence. In some embodiments, the effector protein (e.g., polypeptide) comprises a HEPN domain that is capable of cleaving a target nucleic acid. In some embodiments, the effector protein (e.g., polypeptide) is capable of cleaving a target nucleic acid. In some embodiments, the effector protein (e.g., polypeptide) is capable of cleaving a target nucleic acid or the polypeptide is capable of modifying at least one nucleotide of a target nucleic acid.

In some embodiments, effector proteins disclosed herein provide catalytic activity (e.g., cleavage activity, nickase activity, nuclease activity, other activity, or combinations thereof) similar to that of a naturally-occurring effector protein, such as, for example, a naturally-occurring effector protein with reduced cleavage activity (e.g., Cas 13) including cis cleavage activity, trans cleavage activity, or combinations thereof. In some embodiments, effector proteins disclosed herein is fused to effector partners or fusion proteins, wherein the effector partners or fusion proteins comprise some function or activity not provided by an effector protein.

In some embodiments, an effector protein comprises a CRISPR-associated (“Cas”) protein. In some embodiments, an effector protein functions as a single protein, including a single protein that binds to a guide nucleic acid and modifies a target nucleic acid. Alternatively, in some embodiments, an effector protein interacts with or function with other effector proteins, including one or more of the same effector protein (e.g., dimer), forming a multiprotein complex. In some embodiments, an effector protein, when functioning in a multiprotein complex, comprises only one functional activity (e.g., binding to a guide nucleic acid), while other effector proteins present in the multiprotein complex comprises the other functional activity (e.g., modifying a target nucleic acid). In some embodiments, an effector protein, when functioning in a multiprotein complex, comprises differing and/or complementary functional activity to other effector proteins in the multiprotein complex. Multiprotein complexes, and functions thereof, are described in further detail below. In some embodiments, an effector protein comprises a modified effector protein having increased modification activity and/or increased substrate binding activity (e.g., substrate selectivity, specificity, and/or affinity). Alternatively, or in addition, an effector protein comprises a catalytically inactive effector protein having reduced modification activity or no modification activity.

In some embodiments, effector proteins described herein comprise one or more functional domains. In some embodiments, effector protein functional domains include an oligonucleotide-interacting domain, one or more recognition domains, a non-target strand interacting domain, and a catalytic domain. In some embodiments, the catalytic domain is a HEPN domain. In some embodiments, the effector proteins comprise two HEPN domain. In some embodiments the effector proteins comprise dual HEPN domains capable of cleavage activity.

In some embodiments, an effector protein has a length of at least 600, at least 700, at least 800, at least 900, at least 1,000, at least 1,100, at least 1,200, at least 1,300, at least 1,400, at least 1,500, at least 1,600, or more linked amino acids. In some embodiments, an effector protein has a length of about 600, about 700, about 800, about 900, about 1,000, about 1,100, about 1,200, about 1,300, about 1,400, about 1,500, or about 1,600 linked amino acids.

TABLE 1 provides illustrative amino acid sequences of effector proteins that are useful in the compositions, systems and methods described herein.

In some embodiments, compositions, systems, devices, kits, and methods described herein comprise an effector protein, or a nucleic acid encoding the effector protein, wherein the amino acid sequence of the effector protein comprises at least about 680 contiguous amino acids or more of any one of the amino acid sequences recited in TABLE 1. In some embodiments, the amino acid sequence of an effector protein provided herein comprises at least about 600 contiguous amino acids, at least about 640 contiguous amino acids, at least about 680 contiguous amino acids, at least about 720 contiguous amino acids, at least about 760 contiguous amino acids, at least about 800 contiguous amino acids, at least about 840 contiguous amino acids, at least about 880 contiguous amino acids, at least about 920 contiguous amino acids, at least about 960 contiguous amino acids, at least about 1,000 contiguous amino acids, at least about 1,040 contiguous amino acids, at least about 1,080 contiguous amino acids, at least about 1,120 contiguous amino acids, at least about 1,160 contiguous amino acids, at least about 1,200 contiguous amino acids, at least about 1,240 contiguous amino acids, at least about 1,280 contiguous amino acids, at least about 1,320 contiguous amino acids, at least about 1,360 contiguous amino acids, at least about 1,400 contiguous amino acids, at least about 1,440 contiguous amino acids, at least about 1,480 contiguous amino acids, at least about 1,520 contiguous amino acids, at least about 1,560 contiguous amino acids, at least about 1,600 contiguous amino acids, or more of any one of the amino acid sequences of TABLE 1.

In some embodiments, compositions, systems, devices, kits, and methods described herein comprise an effector protein or a nucleic acid encoding the effector protein, wherein the effector protein comprises a portion of any one of the amino acid sequences recited in TABLE 1. In some embodiments, the effector protein comprises a portion of any one of the amino acid sequences recited in TABLE 1, wherein the portion does not comprise at least the first 10 amino acids, at least the first 20 amino acids, at least the first 40 amino acids, at least the first 60 amino acids, at least the first 80 amino acids, at least the first 100 amino acids, at least the first 120 amino acids, at least the first 140 amino acids, at least the first 160 amino acids, at least the first 180 amino acids, or at least the first 200 amino acids of any one of the amino acid sequences recited in TABLE 1. In some embodiments, the effector protein comprises a portion of any one of the amino acid sequences recited in TABLE 1, wherein the portion does not comprise the last 10 amino acids, the last 20 amino acids, the last 40 amino acids, the last 60 amino acids, the last 80 amino acids, the last 100 amino acids, the last 120 amino acids, the last 140 amino acids, the last 160 amino acids, the last 180 amino acids, or the last 200 amino acids of any one of the amino acid sequences recited in TABLE 1.

In some embodiments, compositions, systems, devices, kits, and methods described herein comprise an effector protein, or a nucleic acid encoding the effector protein, wherein the effector protein comprises an amino acid sequence that is at least 65%, at least 70%, at least 75%, at least 80%, at least 85%, at least 90%, at least 95%, at least 97%, at least 98%, at least 99%, or 100% identical to any one of the amino acid sequences as set forth in TABLE 1. In some embodiments, an effector protein provided herein comprises an amino acid sequence that is at least 65% identical to any one of the amino acid sequences as set forth in TABLE 1. In some embodiments, an effector protein provided herein comprises an amino acid sequence that is at least 70% identical to any one of the amino acid sequences as set forth in TABLE 1. In some embodiments, an effector protein provided herein comprises an amino acid sequence that is at least 75% identical to any one of the amino acid sequences as set forth in TABLE 1. In some embodiments, an effector protein provided herein comprises an amino acid sequence that is at least 80% identical to any one of the amino acid sequences as set forth in TABLE 1. In some embodiments, an effector protein provided herein comprises an amino acid sequence that is at least 85% identical to any one of the amino acid sequences as set forth in TABLE 1. In some embodiments, an effector protein provided herein comprises an amino acid sequence that is at least 90% identical to any one of the amino acid sequences as set forth in TABLE 1. In some embodiments, an effector protein provided herein comprises an amino acid sequence that is at least 95% identical to any one of the amino acid sequences as set forth in TABLE 1. In some embodiments, an effector protein provided herein comprises an amino acid sequence that is at least 97% identical to any one of the amino acid sequences as set forth in TABLE 1. In some embodiments, an effector protein provided herein comprises an amino acid sequence that is at least 98% identical to any one of the amino acid sequences as set forth in TABLE 1. In some embodiments, an effector protein provided herein comprises an amino acid sequence that is at least 99% identical to any one of the amino acid sequences as set forth in TABLE 1. In some embodiments, an effector protein provided herein comprises an amino acid sequence that is identical to any one of the amino acid sequences as set forth in TABLE 1.

In some embodiments, compositions, systems, and methods described herein comprise an effector protein, or a nucleic acid encoding the effector protein, wherein the effector protein comprises an amino acid sequence that is at least 80%, at least 85%, at least 90%, at least 95%, at least 97%, at least 98%, at least 99%, or 100% similar to any one of the amino acid sequences as set forth in TABLE 1. In some embodiments, an effector protein provided herein comprises an amino acid sequence that is at least 80% similar to any one of the amino acid sequences as set forth in TABLE 1. In some embodiments, an effector protein provided herein comprises an amino acid sequence that is at least 85% similar to any one of the amino acid sequences as set forth in TABLE 1. In some embodiments, an effector protein provided herein comprises an amino acid sequence that is at least 90% similar to any one of the amino acid sequences as set forth in TABLE 1. In some embodiments, an effector protein provided herein comprises an amino acid sequence that is at least 95% similar to any one of the amino acid sequences as set forth in TABLE 1. In some embodiments, an effector protein provided herein comprises an amino acid sequence that is at least 97% similar to any one of the amino acid sequences as set forth in TABLE 1. In some embodiments, an effector protein provided herein comprises an amino acid sequence that is at least 98% similar to any one of the amino acid sequences as set forth in TABLE 1. In some embodiments, an effector protein provided herein comprises an amino acid sequence that is at least 99% similar to any one of the amino acid sequences as set forth in TABLE 1. In some embodiments, an effector protein provided herein comprises an amino acid sequence that is 100% similar to any one of the amino acid sequences as set forth in TABLE 1.

In some embodiments, an effector protein provided herein comprises an amino acid sequence that is at least 85% identical to any one of sequences SEQ ID NO: 1-7, 9, 11-15, 68-73, and 76-87 listed in TABLE 1. In some embodiments, an effector protein provided herein comprises an amino acid sequence that is at least 93% identical to SEQ ID NO: 74-75 listed in TABLE 1. In some embodiments, an effector protein provided herein comprises an amino acid sequence that is at least 98% identical to SEQ ID NO: 8 listed in TABLE 1. In some embodiments, an effector protein provided herein comprises an amino acid sequence that is at least 99.5% identical to SEQ ID NO: 10 listed in TABLE 1.

In some embodiments, an effector protein provided herein comprises an amino acid sequence that is at least 89% identical to SEQ ID NO:88, at least 91% identical to SEQ ID NO:89, at least 92% identical to SEQ ID NO: 90 and 92, at least 93% identical to SEQ ID NO: 74-75, at least 94% identical to SEQ ID NO: 91, at least 98% identical to SEQ ID NO: 8, or at least 99.5% identical to SEQ ID NO: 10.

In some embodiments, an effector protein provided herein comprises an amino acid sequence that is at least 85% similar to any one of sequences SEQ ID NO: 1-7, 9, 11-15, 68-73, and 76-87 listed in TABLE 1. In some embodiments, an effector protein provided herein comprises an amino acid sequence that is at least 93% similar to SEQ ID NO: 74-75 listed in TABLE 1. In some embodiments, an effector protein provided herein comprises an amino acid sequence that is at least 98% similar to SEQ ID NO: 8 listed in TABLE 1. In some embodiments, an effector protein provided herein comprises an amino acid sequence that is at least 99.5% similar to SEQ ID NO: 10 listed in TABLE 1.

In some embodiments, an effector protein provided herein comprises an amino acid sequence that is at least 89% similar to SEQ ID NO: 88, at least 91% similar to SEQ ID NO: 89, at least 92% similar to SEQ ID NO: 90 and 92, at least 93% similar to SEQ ID NO: 74-75, at least 94% similar to SEQ ID NO: 91, at least 98% similar to SEQ ID NO: 8, or at least 99.5% similar to SEQ ID NO: 10.

In some embodiments, compositions, systems, devices, kits, and methods described herein comprise an effector protein, or a nucleic acid encoding the effector protein, wherein the effector protein comprises one or more amino acid alterations relative to any one of the amino acid sequences recited in TABLE 1. In some embodiments, the one or more alterations comprises at least one, at least two, at least three, at least four, at least five, at least six, at least seven, at least eight, at least nine, at least ten, at least twelve, at least sixteen, at least twenty, or more amino acid alterations relative to any one of the amino acid sequences recited in TABLE 1. In some embodiments, the one or more alterations comprises one to twenty, one to sixteen, one to twelve, one to eight, one to four, four to twenty, four to sixteen, four to twelve, four to eight, eight to twenty, eight to sixteen, eight to twelve, twelve to twenty, twelve to sixteen, or sixteen to twenty amino acid alterations relative to any one of the amino acid sequences recited in TABLE 1. In some embodiments, the one or more alterations comprises one, two, three, four, five, six, seven, eight, nine, ten, or more amino acid alterations relative to any one of the amino acid sequences recited in TABLE 1. In some embodiments, the effector protein comprising one or more amino acid alterations is a variant of an effector protein described herein. It is understood that any reference to an effector protein herein also refers to an effector protein variant as described herein. In some embodiments, the one or more amino acid alterations comprises conservative substitutions, non-conservative substitutions, deletions, insertions, or combinations thereof.

In some embodiments, compositions, systems, and methods described herein comprise an effector protein, or a nucleic acid encoding the effector protein, wherein the effector protein comprises one or more substitutions relative to any one of the amino acid sequences recited in TABLE 1. In some embodiments, the one or more substitutions comprises at least one, at least two, at least three, at least four, at least five, at least six, at least seven, at least eight, at least nine, at least ten, at least twelve, at least sixteen, at least twenty, or more substitutions relative to any one of the amino acid sequences recited in TABLE 1. In some embodiments, the one or more substitutions comprises one to twenty, one to sixteen, one to twelve, one to eight, one to four, four to twenty, four to sixteen, four to twelve, four to eight, eight to twenty, eight to sixteen, eight to twelve, twelve to twenty, twelve to sixteen, or sixteen to twenty substitutions relative to any one of the amino acid sequences recited in TABLE 1. In some embodiments, the one or more substitutions comprise one, two, three, four, five, six, seven, eight, nine, ten or more substitutions relative to any one of the amino acid sequences recited in TABLE 1. In some embodiments, the one or more substitutions comprise one or more conservative substitutions, one or more non-conservative substitutions, or combinations thereof.

In some embodiments, compositions, systems, and methods described herein comprise an effector protein, or a nucleic acid encoding the effector protein, wherein the effector protein comprises one or more conservative substitutions relative to any one of the amino acid sequences recited in TABLE 1. In some embodiments, the one or more conservative substitutions comprises at least one, at least two, at least three, at least four, at least five, at least six, at least seven, at least eight, at least nine, at least ten, at least twelve, at least sixteen, at least twenty, or more conservative substitutions relative to any one of the amino acid sequences recited in TABLE 1. In some embodiments, the one or more conservative substitutions comprises one to twenty, one to sixteen, one to twelve, one to eight, one to four, four to twenty, four to sixteen, four to twelve, four to eight, eight to twenty, eight to sixteen, eight to twelve, twelve to twenty, twelve to sixteen, or sixteen to twenty conservative substitutions relative to any one of the amino acid sequences recited in TABLE 1. In some embodiments, the one or more conservative substitutions comprise one, two, three, four, five, six, seven, eight, nine, ten or more conservative substitutions relative to any one of the amino acid sequences recited in TABLE 1.

In some embodiments, compositions, systems, and methods described herein comprise an effector protein, or a nucleic acid encoding the effector protein, wherein the effector protein comprises one or more non-conservative substitutions relative to any one of the amino acid sequences recited in TABLE 1. In some embodiments, the one or more non-conservative substitutions comprises at least one, at least two, at least three, at least four, at least five, at least six, at least seven, at least eight, at least nine, at least ten, at least twelve, at least sixteen, at least twenty, or more non-conservative substitutions relative to any one of the amino acid sequences recited in TABLE 1. In some embodiments, the one or more non-conservative substitutions comprises one to twenty, one to sixteen, one to twelve, one to eight, one to four, four to twenty, four to sixteen, four to twelve, four to eight, eight to twenty, eight to sixteen, eight to twelve, twelve to twenty, twelve to sixteen, or sixteen to twenty non-conservative substitutions relative to any one of the amino acid sequences recited in TABLE 1. In some embodiments, the one or more non-conservative substitutions comprise one, two, three, four, five, six, seven, eight, nine, ten or more non-conservative substitutions relative to any one of the amino acid sequences recited in TABLE 1.

In some embodiments, the one or more amino acid alterations result in a change in activity of the effector protein relative to a naturally-occurring counterpart. For example, and as described in further detail below, the one or more amino acid alteration increases or decreases catalytic activity of the effector protein relative to a naturally-occurring counterpart. In another example, the one or more amino acid alteration increases or decreases binding activity of the effector protein relative to a naturally-occurring counterpart. In some embodiments, the one or more amino acid alterations results in a catalytically inactive effector protein variant.

In some embodiments, the one or more amino acid alterations result in a change in activity of the effector protein relative to a naturally-occurring counterpart. For example, and as described in further detail below, the one or more amino acid alteration increases or decreases catalytic activity of the effector protein relative to a naturally-occurring counterpart. In some embodiments, the one or more amino acid alterations results in a catalytically inactive effector protein variant. In some embodiments, the one or more amino acid alteration increases or decreases catalytic activity of the effector protein at elevated temperatures relative to a naturally-occurring counterpart. In some embodiments, the one or more amino acid alteration improves stability and/or manufacturability (e.g., expressibility, solubility, purification, etc.) of the effector protein relative to a naturally-occurring counterpart.

In some embodiments, compositions, systems, and methods described herein comprise an effector protein or a nucleic acid encoding the effector protein, wherein the effector protein comprises one or more amino acid alterations relative to any one of the amino acid sequences recited in TABLE 1. In some embodiments, an effector protein comprising one or more amino acid alterations is a variant of an effector protein described herein. It is understood that any reference to an effector protein herein also refers to an effector protein variant as described herein.

In some embodiments, an effector protein or a nucleic acid encoding the effector protein comprises 1 amino acid alteration, 2 amino acid alterations, 3 amino acid alterations, 4 amino acid alterations, 5 amino acid alterations, 6 amino acid alterations, 7 amino acid alterations, 8 amino acid alterations, 9 amino acid alterations, 10 amino acid alterations or more relative to any one of the amino acid sequences recited in TABLE 1. In some embodiments, the one or more amino acid alterations comprises one or more amino acid substitutions, amino acid deletions, amino acid insertions, or a combination thereof.

In some embodiments, amino acid sequences comprised in effector proteins described herein comprise one or more amino acid alterations relative to a reference sequence, such as an amino acid sequence comprised in a polypeptide described herein or in a corresponding WT. In some embodiments, amino acid sequences comprised in effector proteins described herein comprise one or more amino acid alterations relative to a reference sequence, wherein other than the one or more amino acid alterations, the amino acid sequence comprised in the effector protein is at least 85%, at least 90%, at least 95%, at least 98%, at least 99%, or is identical to the reference sequence, such as any one of the amino acid sequences recited in TABLE 1. In some embodiments, amino acid sequences comprised in effector proteins described herein comprise one or more amino acid alterations relative to a reference sequence, wherein other than the one or more amino acid alterations, the amino acid sequence comprised in the effector protein is at least 85%, at least 90%, at least 95%, at least 98%, at least 99%, or 100% similar to the reference sequence, such as any one of the amino acid sequences recited in TABLE 1.

In some embodiments, an effector protein described herein has a variant amino acid sequence of any one of the amino acid sequences recited in TABLE 1 comprising one or more amino acid alterations as described herein, wherein other than the one or more amino acid alterations, the amino acid sequence is at least 85%, at least 90%, at least 95%, at least 98%, at least 99%, or is identical to any one of the amino acid sequences recited in TABLE 1. In some embodiments, an effector protein described herein has a variant amino acid sequence of SEQ ID NO: 69 and comprises one or more amino acid alterations as described herein, wherein other than the one or more amino acid alterations, the amino acid sequence is at least 85%, at least 90%, at least 95%, at least 98%, at least 99%, or is identical to SEQ ID NO: 69. In some embodiments, an effector protein described herein has a variant amino acid sequence of SEQ ID NO: 69 and comprises one or more amino acid alterations as described herein, wherein other than the one or more amino acid alterations, the amino acid sequence is at least 85%, at least 90%, at least 95%, at least 98%, at least 99%, or is 100% similar to SEQ ID NO: 69. When describing an amino acid sequence of an effector protein with amino acid alteration(s) herein, a person of ordinary skill in the art understands that reference of the one or more amino acid alterations at the positions described herein (e.g., described in TABLE 1.1), and the percent identity to a reference sequence (e.g., SEQ ID NO: 69) describes an amino acid sequence of the effector protein, such that the effector protein has an amino acid sequence with a certain percent identity or similarity to the reference sequence while retaining the one or more amino acid alterations that the effector protein is described as having.

In some embodiments, the amino acid sequence of an effector protein described herein is a variant of any one of the amino acid sequences recited in TABLE 1 comprising one or more amino acid alterations as described herein, wherein other than the one or more amino acid alterations, the amino acid sequence is at least 85%, at least 90%, at least 95%, at least 98%, at least 99%, or is identical to any one of the amino acid sequences recited in TABLE 1. In some embodiments, the amino acid sequence of an effector protein described herein is a variant of SEQ ID NO: 69 and comprises one or more amino acid alterations as described herein, wherein other than the one or more amino acid alterations, the amino acid sequence is at least 85%, at least 90%, at least 95%, at least 98%, at least 99%, or is identical to SEQ ID NO: 69. In some embodiments, the amino acid sequence of an effector protein described herein is a variant of SEQ ID NO: 69 and comprises one or more amino acid alterations as described herein, wherein other than the one or more amino acid alterations, the amino acid sequence is at least 85%, at least 90%, at least 95%, at least 98%, at least 99%, or is 100% similar to SEQ ID NO: 69.

In some embodiments, one or more amino acid alterations are located in one or more regions that interact with a substrate, such as a target nucleic acid, an engineered guide nucleic acid, or a guide nucleic acid-target nucleic acid heteroduplex. In some embodiments, one or more amino acid alterations are located in a region of the effector protein that comprises a substrate binding activity, a catalytic activity, and/or a binding affinity for a substrate, such as a target nucleic acid, an engineered guide nucleic acid, or a guide nucleic acid-target nucleic acid heteroduplex. In some embodiments, one or more amino acid alterations are located in a HEPN domain (e.g., HEPN-1 and HEPN-2).

In some embodiments, the one or more amino acid alterations can be located atone or more residues corresponding to one or more positions relative to a reference sequence, such as any one of the one of the amino acid sequences recited in TABLE 1. As used herein, the phrase “a residue corresponding to any one of the positions listed in TABLE 1.1 relative to SEQ ID NO: 69”, or grammatical equivalents thereof, refers to a residue at a corresponding position following an alignment of two sequences. In some embodiments, the reference sequence is an effector protein that is not SEQ ID NO: 69. In some embodiments, the one or more amino acid alterations are of one or more residues located at one or more positions described in TABLE 1.1, relative to a reference sequence, such as any one of the one of the amino acid sequences recited in TABLE 1.

In some embodiments, an effector protein provided herein is a variant of a reference polypeptide, wherein the reference polypeptide has an amino acid sequence of SEQ ID NO: 69, and the effector protein has one or more amino acid alterations at one or more residues corresponding to one or more positions described in TABLE 1.1 relative to SEQ ID NO: 69. In some embodiments, an effector protein provided herein is a variant of a reference polypeptide, wherein the reference polypeptide has an amino acid sequence of any one of the sequences set forth in TABLE 1, and the effector protein has one or more amino acid alterations at one or more residues corresponding to one or more positions described in TABLE 1.1 relative to the reference sequence. In some embodiments, an effector protein provided herein is a variant of a reference polypeptide, wherein the reference polypeptide has an amino acid sequence of SEQ ID NO: 69, and the effector protein has one or more amino acid alterations at one or more residues independently corresponding to one or more positions selected from: 9, 15, 56, 106, 121, 125, 131, 139, 150, 154, 164, 166, 175, 184, 198, 200, 242, 247, 262, 265, 281, 289, 305, 311, 313, 314, 318, 333, 338, 352, 372, 381, 480, 485, 492, 496, 501, 517, 521, 537, 543, 546, 547, 548, 555, 559, 567, 569, 574, 579, 585, 618, 621, 622, 623, 631, 647, 656, 684, 705, 709, 717, 722, 726, 737, 747, 762, 765, 766, 769, 789, 790, 800, 801, 807, 819, 827, 836, 843, 846, 847, 857, 858, 864, 867, 870, 871, 909, 915, 919, 923, 927, 974, 1011, 1020, 1030, 1032, 1035, 1049, 1054, 1056, 1062, 1064, 1083, 1085, or a combination thereof, relative to SEQ ID NO: 69. In some embodiments, an effector protein provided herein is a variant of a reference polypeptide, wherein the reference polypeptide has an amino acid sequence of SEQ ID NO: 69, and the effector protein has one or more amino acid alterations at one or more residues independently corresponding to one or more positions selected from: 121, 139, 311, 184, 154, 547, 318, 656, 372, 858, 548, 352, 927, 737, 1062, 819, 501, 974, 1064, 722, 621, 765, 622, 807, 762, 871, 800, 827, 1020, or a combination thereof, relative to SEQ ID NO: 69.

In some embodiments, the one or more amino acid alterations comprise one or more deletions, insertions, substitutions, or a combination thereof. In some embodiments, the one or more amino acid substitutions comprise a conservative or a non-conversative substitution. As a non-limiting example, a conservative substitution of a basic amino acid involves substitution for another basic (positively charged) amino acid (e.g., Lys (K), Arg (R), or His (H)). As a non-limiting example, a non-conservative substitution of a basic amino acid involves substitution for an acidic (negatively charged) amino acid (e.g., Asp (D) or Glu (E)). In some embodiments, each one or more amino acid alteration is independently a conservative or non-conservative substitution.

In some embodiments, each of the one or more amino acid alterations is independently a substitution with a basic (positively charged) amino acid, an acidic (negatively-charged) amino acid, a non-polar (hydrophobic) amino acid, or an uncharged polar amino acid, or a combination thereof.

In some embodiments, each one or more amino acid alterations is independently a substitution with a basic (positively charged) amino acid selected from a group comprising: Lys (K), Arg (R), His (H), or combinations thereof. In some embodiments, each one or more amino acid alterations is independently a substitution with a basic (positively charged) amino acid selected from a group comprising: Lys (K), Arg (R), or combinations thereof. In some embodiments, each one or more amino acid alterations is independently a substitution of an amino acid residue with a Lys (K), Arg (R), or His (H). In some embodiments, a substitution with a basic (positively charged) amino acid is a substitution of an amino acid residue with a Lys (K), Arg (R), or His (H).

In some embodiments, each one or more amino acid alterations is independently a substitution with an acidic (negatively charged) amino acid selected from a group comprising: Asp (D), Glu (E), or combinations thereof. In some embodiments, each one or more amino acid alterations is independently a substitution with an acidic (negatively charged) amino acid comprising Glu (E). In some embodiments, each one or more amino acid alterations is independently a substitution of an amino acid residue with an Asp (D) or Glu (E). In some embodiments, a substitution with an acidic (negatively charged) amino acid is a substitution of an amino acid residue with an Asp (D) or Glu (E).

In some embodiments, each one or more amino acid alterations is independently a substitution with a non-polar (hydrophobic) amino acid selected from a group comprising: Cys (C), Ala (A), Val (V), Leu (L), Ile (I), Pro (P), Phe (F), Met (M), Trp (W), Gly (G), Tyr (Y), or combinations thereof. In some embodiments, each one or more amino acid alterations is independently a substitution with a non-polar (hydrophobic) amino acid selected from a group comprising: Cys (C), Ala (A), Val (V), Leu (L), Ile (I), Pro (P), Gly (G), Tyr (Y), or combinations thereof. In some embodiments, each one or more amino acid alterations is independently a substitution of an amino acid residue with a Cys (C), Ala (A), Val (V), Leu (L), Ile (I), Pro (P), Phe (F), Met (M), Trp (W), Gly (G), or Tyr (Y). In some embodiments, a substitution with a non-polar (hydrophobic) amino acid is a substitution of an amino acid residue with a Cys (C), Ala (A), Val (V), Leu (L), Ile (I), Pro (P), Phe (F), Met (M), Trp (W), Gly (G), or Tyr (Y).

In some embodiments, each one or more amino acid alterations is independently a substitution with an uncharged polar amino acid selected from a group comprising: Asn (N), Gln (Q), Ser (S), Thr (T), or combinations thereof. In some embodiments, each one or more amino acid alterations is independently a substitution of an amino acid residue with an Asn (N), Gln (Q), Ser (S), or Thr (T). In some embodiments, a substitution with an uncharged polar amino acid is a substitution of an amino acid residue with an Asn (N), Gln (Q), Ser (S), or Thr (T).

In some embodiments, the one or more amino acid alterations are each a substitution of an amino acid residue with a basic (positively charged) amino acid, an acidic (negatively charged) amino acid, a non-polar (hydrophobic) amino acid, or an uncharged polar amino acid. In some embodiments, the one or more amino acid alterations are independently selected from the amino acid alterations recited in TABLE 1.1, or a combination thereof are relative to the corresponding amino acid sequence referenced in TABLE 1.1.

An effector protein provided herein can comprise a combination of 2, 3, 4, 5, 6, 7, 8, 9, 10, 11, 12, 13, 14, 15, 16, 17, 18, 19, 20, 21, 22, 23, 24, 25, 26, 27, 28, 29, 30, or more, including up to an amino acid alteration at all of the positions identified in TABLE 1.1 relative to a reference sequence (e.g., SEQ ID NO: 69). In some embodiments, an effector protein described herein has a combination of amino acid alterations comprising a first amino acid alteration at a residue corresponding to any position recited in TABLE 1.1 relative to SEQ ID NO: 69, and one or more amino acid alterations, for example, one or more conservative or non-conservative substitutions. In some embodiments, an effector protein described herein has a combination of amino acid alterations comprising a first amino acid alteration at a residue corresponding to any position recited in TABLE 1.1 relative to SEQ ID NO: 69, and one or more amino acid alterations at one or more residues corresponding to any position recited in TABLE 1.1 relative to SEQ ID NO: 69 that is not at the position of the first amino acid alteration. For example, an effector protein described herein has a combination of amino acid alterations comprising a first amino acid alteration at one or more residues independently corresponding to one or more positions selected from: 9, 15, 56, 106, 121, 125, 131, 139, 150, 154, 164, 166, 175, 184, 198, 200, 242, 247, 262, 265, 281, 289, 305, 311, 313, 314, 318, 333, 338, 352, 372, 381, 480, 485, 492, 496, 501, 517, 521, 537, 543, 546, 547, 548, 555, 559, 567, 569, 574, 579, 585, 618, 621, 622, 623, 631, 647, 656, 684, 705, 709, 717, 722, 726, 737, 747, 762, 765, 766, 769, 789, 790, 800, 801, 807, 819, 827, 836, 843, 846, 847, 857, 858, 864, 867, 870, 871, 909, 915, 919, 923, 927, 974, 1011, 1020, 1030, 1032, 1035, 1049, 1054, 1056, 1062, 1064, 1083, 1085, or combinations thereof relative to SEQ ID NO: 69, and one or more amino acid alterations at one or more residues independently corresponding to one or more positions selected from: 9, 15, 56, 106, 121, 125, 131, 139, 150, 154, 164, 166, 175, 184, 198, 200, 242, 247, 262, 265, 281, 289, 305, 311, 313, 314, 318, 333, 338, 352, 372, 381, 480, 485, 492, 496, 501, 517, 521, 537, 543, 546, 547, 548, 555, 559, 567, 569, 574, 579, 585, 618, 621, 622, 623, 631, 647, 656, 684, 705, 709, 717, 722, 726, 737, 747, 762, 765, 766, 769, 789, 790, 800, 801, 807, 819, 827, 836, 843, 846, 847, 857, 858, 864, 867, 870, 871, 909, 915, 919, 923, 927, 974, 1011, 1020, 1030, 1032, 1035, 1049, 1054, 1056, 1062, 1064, 1083, 1085, or combinations thereof relative to SEQ ID NO: 69. In some embodiments, an effector protein described herein has a combination of amino acid alterations comprising two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty one or more, twenty two or more, or twenty three or more amino acid alterations each corresponding to any differing position as recited in TABLE 1.1 relative to SEQ ID NO: 69. Combinations of exemplary amino acid alteration may each be independently a conservative substitution or a non-conservative substitution.

In some embodiments, the one or more amino acid alterations may result in a change in activity of the effector protein relative to a naturally-occurring counterpart effector protein (e.g., SEQ ID NO: 69) or reference sequence (e.g., SEQ ID NO: 51). For example, and as described in further detail below, the one or more amino acid alteration increases or decreases catalytic activity of the effector protein relative to a naturally-occurring counterpart effector protein (e.g., SEQ ID NO: 51) or reference sequence (e.g., SEQ ID NO: 69). In some embodiments, the one or more amino acid alterations results in a catalytically inactive effector protein variant. Catalytically inactive effect protein variants are further described herein. In some embodiments, the effector proteins comprising the one or more amino acid alterations can carry out a similar enzymatic reaction as the naturally-occurring counterpart effector protein (e.g., SEQ ID NO: 51) or reference sequence (e.g., SEQ ID NO: 69).

In some embodiments, the variants of the effector protein as described herein can include alterations that provide a beneficial characteristic to effector proteins described herein, including but not limited to, increased activity (e.g., indel activity, catalytic activity, specificity or selectivity and/or affinity for a substrate, such as a target nucleic acid and/or a guide nucleic acid). In some embodiments, variants of effector proteins described herein can exhibit an activity that is at least the same or higher than the naturally-occurring counterpart effector protein (e.g., SEQ ID NO: 51) or reference sequence (e.g., SEQ ID NO: 69), that is, it has one or more activities that are the same or higher than the effector protein (e.g., SEQ ID NO: 51) or reference sequence (e.g., SEQ ID NO: 69) without the variant at the same amino acid position(s). For example, variants can have one or more activity that is at least 10%, at least 20%, at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, or at least 200% higher over a naturally-occurring effector protein (e.g., SEQ ID NO: 51) or reference sequence (e.g., SEQ ID NO: 69). In some embodiments, activity of effector proteins described herein or variants thereof can be measured relative to a naturally-occurring effector protein (e.g., SEQ ID NO:51) or reference sequence (e.g., SEQ ID NO: 69) in a cleavage assay, such as those described herein (see, e.g., Examples 2-5 and 9-17).

In some embodiments, the one or more amino acid alterations are independently selected from the amino acid alterations recited in TABLE 1.1, or a combination thereof are relative SEQ ID NO: 69. In some embodiments, each of the one or more amino acid alterations are independently selected from: T9G, T15K, Q56A, H106Q, E121N, C125L, E131K, H139Q, N150Y, G154K, H164R, Q166K, Q175K, D184E, E198K, F200Y, A242M, R247T, A262T, N265R, N281K, D289T, H305K, M311V, A313S, N314K, K318Q, E333H, L338I, S352C, V372L, L381M, Q480Y, Q485S, V492Q, H496S, V501T, G517N, S521A, L537R, E543L, W546Y, S547N, G548A, I555L, Y559N, N567S, D569H, D574Q, L579V, Q585L, Q618E, W621Q, I622N, I622K, M623L, D631C, L647V, M656L, L684K, A705T, Q709H, K717M, N722R, T726Q, A737S, T747L, A762G, W765R, W765N, Q766M, K769E, T789Q, N790K, D800K, D800G, D800R, E801M, E801G, S807T, S807R, S819K, S827R, S827K, N836P, N843S, A846P, T847K, E857M, Y858L, E864Q, E867A, D870E, N871K, N909D, N909E, E915V, S919Q, S919K, I923L, M927I, D974Y, L1011V, H1020R, A1030S, T1032V, D1035M, D1049G, Q1054T, Q1056L, S1062P, I1064K, P1083Q, A1085E, or combinations thereof, relative to SEQ ID NO: 69.

In some embodiments, each of the one or more amino acid alterations are independently selected from: E121N, H139Q, M311V, D184E, G154K, S547N, K318Q, M656L, V372L, Y858L, G548A, S352C, M927I, A737S, S1062P, S819K, V501T, D974Y, I1064K, N722R, W621Q, W765N, I622N, S807R, A762G, N871K, D800R, S827K, and H1020R, or combinations thereof, relative to SEQ ID NO: 69. In some embodiments, the one or more amino acid alterations comprise: E121N, M311V, S547N, M656L, Y858L, M927I, and S1062P relative to SEQ ID NO: 69. In some embodiments, the one or more amino acid alterations comprise: H139Q, D184E, K318Q, V372L, G548A, and A737S relative to SEQ ID NO: 69. In some embodiments, the one or more amino acid alterations comprise: H139Q, D184E, K318Q, V372L, G548A, A737S, S819K, D974Y, and I1064K relative to SEQ ID NO: 69. In some embodiments, the one or more amino acid alterations comprise: H139Q, D184E, M311V, K318Q, S352C, V372L, V501T, G548A, N722R, A737S, W765N, S807R, S819K, N871K, D974Y, and I1064K relative to SEQ ID NO: 69. In some embodiments, the one or more amino acid alterations comprise: H139Q, G154K, D184E, M311V, K318Q, S352C, V372L, V501T, G548A, I622N, N722R, A737S, W765N, S807R, S819K, S827K, N871K, D974Y, H1020R, and I1064K relative to SEQ ID NO: 69. In some embodiments, the one or more amino acid alterations comprise: H139Q, D184E, M311V, K318Q, S352C, V372L, V501T, G548A, W621Q, I622N, N722R, A737S, A762G, W765N, D800R, S807R, S819K, N871K, D974Y, and I1064K relative to SEQ ID NO: 69.

In some embodiments, an effector protein described herein has a variant amino acid sequence of SEQ ID NO: 69 and comprises one or more amino acid alterations, each a substitution of an amino acid residue with an amino acid residue selected from a group comprising: Asn (N), Gln (Q), Val (V), Glu (E), Lys (K), Leu (L), Ala (A), Cys (C), Ile (I), Ser (S), Pro (P), Thr (T), Tyr (Y), Arg (R), Gly (G), or combinations thereof, wherein other than the one or more amino acid alterations, the amino acid sequence is at least 85%, at least 90%, at least 95%, at least 98%, at least 99%, or 100% identical to SEQ ID NO: 69. In some embodiments, an effector protein described herein has a variant amino acid sequence of SEQ ID NO: 69 and comprises one or more amino acid alterations, each a substitution of an amino acid residue with an amino acid residue selected from a group comprising: Asn (N), Gln (Q), Val (V), Glu (E), Lys (K), Leu (L), Ala (A), Cys (C), Ile (I), Ser (S), Pro (P), Thr (T), Tyr (Y), Arg (R), Gly (G), or combinations thereof, wherein other than the one or more amino acid alterations, the amino acid sequence is at least 85%, at least 90%, at least 95%, at least 98%, at least 99%, or 100% similar to SEQ ID NO: 69.

In some embodiments, an effector protein, or a nucleic acid encoding the effector protein, comprises a variant amino acid sequence of SEQ ID NO: 69, or a functional fragment thereof, wherein the variant amino acid sequence comprises one or more amino acid alterations at one or more residues corresponding to one or more positions listed in TABLE 1.1; and optionally wherein the amino acid sequence, other than the one or more amino acid alterations, has at least 85% sequence identity to the amino acid sequence referenced in SEQ ID NO: 69. In some embodiments, an effector protein, or a nucleic acid encoding the effector protein, comprises a variant amino acid sequence of SEQ ID NO: 69, or a functional fragment thereof, wherein the variant amino acid sequence comprises one or more amino acid alterations at one or more residues corresponding to one or more positions listed in TABLE 1.1; and optionally wherein the amino acid sequence, other than the one or more amino acid alterations, has at least 85% sequence similarity to the amino acid sequence referenced in SEQ ID NO: 69.

A person of ordinary skill in the art would understand that the one or more amino acid alterations described herein are exemplary. Without being bound by theory, in some embodiments, a polypeptide comprising the one or more amino acid alterations described herein (e.g., described in at least TABLE 1.1) or a nucleic acid encoding the polypeptide comprising the one or more amino acid alterations described herein are understood to be exemplary.

In some embodiments, a polypeptide (e.g., effector protein), or a nucleic acid encoding the polypeptide (e.g., effector protein), comprises at least about 680 contiguous amino acids or more of any one of the amino acid sequences recited in TABLE 1.2. In some embodiments, the amino acid sequence of an effector protein provided herein comprises at least about 600 contiguous amino acids, at least about 640 contiguous amino acids, at least about 680 contiguous amino acids, at least about 720 contiguous amino acids, at least about 760 contiguous amino acids, at least about 800 contiguous amino acids, at least about 840 contiguous amino acids, at least about 880 contiguous amino acids, at least about 920 contiguous amino acids, at least about 960 contiguous amino acids, at least about 1,000 contiguous amino acids, at least about 1,040 contiguous amino acids, at least about 1,080 contiguous amino acids, at least about 1,120 contiguous amino acids, at least about 1,160 contiguous amino acids, at least about 1,200 contiguous amino acids, at least about 1,240 contiguous amino acids, at least about 1,280 contiguous amino acids, at least about 1,320 contiguous amino acids, at least about 1,360 contiguous amino acids, at least about 1,400 contiguous amino acids, at least about 1,440 contiguous amino acids, at least about 1,480 contiguous amino acids, at least about 1,520 contiguous amino acids, at least about 1,560 contiguous amino acids, at least about 1,600 contiguous amino acids, or more of any one of the amino acid sequences of TABLE 1.2.

In some embodiments, a polypeptide (e.g., effector protein) or a nucleic acid encoding the polypeptide (e.g., effector protein), comprises a portion of any one of the amino acid sequences recited in TABLE 1.2. In some embodiments, the effector protein comprises a portion of any one of the amino acid sequences recited in TABLE 1.2, wherein the portion does not comprise at least the first 10 amino acids, at least the first 20 amino acids, at least the first 40 amino acids, at least the first 60 amino acids, at least the first 80 amino acids, at least the first 100 amino acids, at least the first 120 amino acids, at least the first 140 amino acids, at least the first 160 amino acids, at least the first 180 amino acids, or at least the first 200 amino acids of any one of the amino acid sequences recited in TABLE 1.2. In some embodiments, the effector protein comprises a portion of any one of the amino acid sequences recited in TABLE 1.2, wherein the portion does not comprise the last 10 amino acids, the last 20 amino acids, the last 40 amino acids, the last 60 amino acids, the last 80 amino acids, the last 100 amino acids, the last 120 amino acids, the last 140 amino acids, the last 160 amino acids, the last 180 amino acids, or the last 200 amino acids of any one of the amino acid sequences recited in TABLE 1.2.

In some embodiments, a polypeptide (e.g., effector protein), or a nucleic acid encoding the polypeptide (e.g., effector protein), comprises an amino acid sequence that is at least 85%, at least 90%, at least 95%, at least 97%, at least 98%, at least 99%, or 100% identical to any one of the amino acid sequences as set forth in TABLE 1.2. In some embodiments, an effector protein provided herein comprises an amino acid sequence that is at least 85% identical to any one of the amino acid sequences as set forth in TABLE 1.2. In some embodiments, an effector protein provided herein comprises an amino acid sequence that is at least 90% identical to any one of the amino acid sequences as set forth in TABLE 1.2. In some embodiments, an effector protein provided herein comprises an amino acid sequence that is at least 95% identical to any one of the amino acid sequences as set forth in TABLE 1.2. In some embodiments, an effector protein provided herein comprises an amino acid sequence that is at least 97% identical to any one of the amino acid sequences as set forth in TABLE 1.2. In some embodiments, an effector protein provided herein comprises an amino acid sequence that is at least 98% identical to any one of the amino acid sequences as set forth in TABLE 1.2. In some embodiments, an effector protein provided herein comprises an amino acid sequence that is at least 99% identical to any one of the amino acid sequences as set forth in TABLE 1.2. In some embodiments, an effector protein provided herein comprises an amino acid sequence that is 100% identical to any one of the amino acid sequences as set forth in TABLE 1.2.

In some embodiments, a polypeptide (e.g., effector protein), or a nucleic acid encoding the polypeptide (e.g., effector protein), comprises an amino acid sequence that is at least 85%, at least 90%, at least 95%, at least 97%, at least 98%, at least 99%, or 100% similar to any one of the amino acid sequences as set forth in TABLE 1.2. In some embodiments, an effector protein provided herein comprises an amino acid sequence that is at least 85% similar to any one of the amino acid sequences as set forth in TABLE 1.2. In some embodiments, an effector protein provided herein comprises an amino acid sequence that is at least 90% similar to any one of the amino acid sequences as set forth in TABLE 1.2. In some embodiments, an effector protein provided herein comprises an amino acid sequence that is at least 95% similar to any one of the amino acid sequences as set forth in TABLE 1.2. In some embodiments, an effector protein provided herein comprises an amino acid sequence that is at least 97% similar to any one of the amino acid sequences as set forth in TABLE 1.2. In some embodiments, an effector protein provided herein comprises an amino acid sequence that is at least 98% similar to any one of the amino acid sequences as set forth in TABLE 1.2. In some embodiments, an effector protein provided herein comprises an amino acid sequence that is at least 99% similar to any one of the amino acid sequences as set forth in TABLE 1.2. In some embodiments, an effector protein provided herein comprises an amino acid sequence that is 100% similar to any one of the amino acid sequences as set forth in TABLE 1.2.

In some embodiments, the effector protein provided herein comprises an amino acid sequence that is at least 85% identical to any one of SEQ ID NO: 119-282 listed in TABLE 1.2. In some embodiments, the polypeptide comprises an amino acid sequence that is at least 85% identical to any one of SEQ ID NO: 119-282 listed in TABLE 1.2. In some embodiments, an effector protein provided herein comprises an amino acid sequence that is at least 85% similar to any one of SEQ ID NO: 119-282 listed in TABLE 1.2. In some embodiments, the polypeptide comprises an amino acid sequence that is at least 85% similar to any one of SEQ ID NO: 119-282 listed in TABLE 1.2. In some embodiments, the effector protein described herein is complexed and/or interacts with a guide nucleic acid or an engineered guide nucleic acid, or a nucleic acid that encodes the guide nucleic acid or the engineered guide nucleic acid. In some embodiments a recombinant nucleic acid encodes the effector protein described herein.

In some embodiments, an effector protein, or a nucleic acid encoding the effector protein, comprises one or more amino acid alterations relative to any one of the amino acid sequences recited in TABLE 1.2. In some embodiments, the one or more alterations comprises at least one, at least two, at least three, at least four, at least five, at least six, at least seven, at least eight, at least nine, at least ten, at least twelve, at least sixteen, at least twenty, or more amino acid alterations relative to any one of the amino acid sequences recited in TABLE 1.2. In some embodiments, the one or more alterations comprises one to twenty, one to sixteen, one to twelve, one to eight, one to four, four to twenty, four to sixteen, four to twelve, four to eight, eight to twenty, eight to sixteen, eight to twelve, twelve to twenty, twelve to sixteen, or sixteen to twenty amino acid alterations relative to any one of the amino acid sequences recited in TABLE 1.2. In some embodiments, the one or more alterations comprises one, two, three, four, five, six, seven, eight, nine, ten, or more amino acid alterations relative to any one of the amino acid sequences recited in TABLE 1.2. In some embodiments, the effector protein comprising one or more amino acid alterations is a variant of an effector protein described herein. It is understood that any reference to an effector protein herein also refers to an effector protein variant as described herein. In some embodiments, the one or more amino acid alterations comprises conservative substitutions, non-conservative substitutions, deletions, insertions, or combinations thereof.

In some embodiments, an effector protein, or a nucleic acid encoding the effector protein, comprises one or more substitutions relative to any one of the amino acid sequences recited in TABLE 1.2. In some embodiments, the one or more substitutions comprises at least one, at least two, at least three, at least four, at least five, at least six, at least seven, at least eight, at least nine, at least ten, at least twelve, at least sixteen, at least twenty, or more substitutions relative to any one of the amino acid sequences recited in TABLE 1.2. In some embodiments, the one or more substitutions comprises one to twenty, one to sixteen, one to twelve, one to eight, one to four, four to twenty, four to sixteen, four to twelve, four to eight, eight to twenty, eight to sixteen, eight to twelve, twelve to twenty, twelve to sixteen, or sixteen to twenty substitutions relative to any one of the amino acid sequences recited in TABLE 1.2. In some embodiments, the one or more substitutions comprise one, two, three, four, five, six, seven, eight, nine, ten or more substitutions relative to any one of the amino acid sequences recited in TABLE 1.2. In some embodiments, the one or more substitutions comprise one or more conservative substitutions, one or more non-conservative substitutions, or combinations thereof.

In some embodiments, an effector protein, or a nucleic acid encoding the effector protein, comprises one or more conservative substitutions relative to any one of the amino acid sequences recited in TABLE 1.2. In some embodiments, the one or more conservative substitutions comprises at least one, at least two, at least three, at least four, at least five, at least six, at least seven, at least eight, at least nine, at least ten, at least twelve, at least sixteen, at least twenty, or more conservative substitutions relative to any one of the amino acid sequences recited in TABLE 1.2. In some embodiments, the one or more conservative substitutions comprises one to twenty, one to sixteen, one to twelve, one to eight, one to four, four to twenty, four to sixteen, four to twelve, four to eight, eight to twenty, eight to sixteen, eight to twelve, twelve to twenty, twelve to sixteen, or sixteen to twenty conservative substitutions relative to any one of the amino acid sequences recited in TABLE 1.2. In some embodiments, the one or more conservative substitutions comprise one, two, three, four, five, six, seven, eight, nine, ten or more conservative substitutions relative to any one of the amino acid sequences recited in TABLE 1.2.

In some embodiments, an effector protein, or a nucleic acid encoding the effector protein, comprises one or more non-conservative substitutions relative to any one of the amino acid sequences recited in TABLE 1.2. In some embodiments, the one or more non-conservative substitutions comprises at least one, at least two, at least three, at least four, at least five, at least six, at least seven, at least eight, at least nine, at least ten, at least twelve, at least sixteen, at least twenty, or more non-conservative substitutions relative to any one of the amino acid sequences recited in TABLE 1.2. In some embodiments, the one or more non-conservative substitutions comprises one to twenty, one to sixteen, one to twelve, one to eight, one to four, four to twenty, four to sixteen, four to twelve, four to eight, eight to twenty, eight to sixteen, eight to twelve, twelve to twenty, twelve to sixteen, or sixteen to twenty non-conservative substitutions relative to any one of the amino acid sequences recited in TABLE 1.2. In some embodiments, the one or more non-conservative substitutions comprise one, two, three, four, five, six, seven, eight, nine, ten or more non-conservative substitutions relative to any one of the amino acid sequences recited in TABLE 1.2.

### Engineered Proteins

In some embodiments, proteins or polypeptides (e.g., effector proteins or fusion partners) described herein have been modified (also referred to as an engineered protein or engineered polypeptide). In some embodiments, a modification of the effector proteins includes addition of one or more amino acids, deletion of one or more amino acids, substitution of one or more amino acids, or combinations thereof. In some embodiments, effector proteins disclosed herein are engineered proteins. Unless otherwise indicated, reference to effector proteins throughout the present disclosure include engineered proteins thereof.

In some embodiments, engineered effector proteins as described herein comprise one or more amino acid modifications relative to cognate effector protein (e.g., a modification as exemplified when comparing to an effector protein having any one of the amino acid sequences recited in TABLE 1 to the cognate effector protein), and wherein the engineered effector protein exhibits one or more improved characteristics compared to the cognate effector protein (e.g., a naturally occurring effector protein). In some embodiments, a cognate effector protein, as described herein, refers to a naturally occurring effector protein that may be used as the parental effector protein sequence for protein engineering. In some embodiments, the naturally occurring effector protein comprises certain characteristics (e.g., structure and/or activity) that may be of interest for protein engineering.

In some embodiments, the one or more improved characteristics of the engineered effector protein compared to the cognate effector protein include, but are not limited to: increased catalytic activity at a temperature above 37° C.; increased catalytic activity at a defined salt concentration; increased editing of target nucleic acids; increased cleavage rate of target nucleic acids; increased trans cleavage rate; increased formation of a complex comprising the engineered polypeptide and an engineered guide nucleic acid; increased solubility; increased stability; increased manufacturability (e.g., increased expressibility, solubility, purification, etc.), increased binding affinity to the guide nucleic acid; increased binding affinity to the target nucleic acid; increased editing efficiency; increased editing specificity; increased or decreased target strand loading for double strand cleavage; increased or decreased target strand loading for single strand nicking; decreased off-target cleavage; increased binding of the non-target strand of DNA; or combinations thereof.

In some embodiments, the complex comprising the engineered polypeptide and an engineered guide nucleic acid comprises increased stability as compared to a complex comprising the cognate effector protein and an engineered guide nucleic acid. In some embodiments, the one or more improved characteristics of the engineered effector protein compared to the cognate effector protein are selected from: increased catalytic activity at a temperature above 37° C.; increased catalytic activity at a defined salt concentration; increased editing of target nucleic acid; increased cleavage rate of target nucleic acid; increased trans cleavage rate; increased formation of a complex comprising the engineered polypeptide and an engineered guide nucleic acid; increased solubility; increased manufacturability (e.g., increased expressibility, solubility, purification, etc.), and increased stability.

In some embodiments, the one or more of the improved characteristics of the engineered effector protein is at least about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 97%, about 98%, about 99%, or about 100% improved relative to the cognate effector protein when assayed in a comparable fashion and/or via the same assay wherein the assay is an appropriate assay known in the art. In some embodiments, the one or more of the improved characteristics of the engineered effector protein is at least about 1 to about 100,000-fold improved relative to the cognate effector protein when assayed in a comparable fashion. In some embodiments, the one or more of the improved characteristics of the engineered effector protein is at least about 1.1 to about 100,000-fold improved relative to the cognate effector protein when assayed in a comparable fashion and/or via the same assay wherein the assay is an appropriate assay known in the art. In other embodiments, the improvement is at least about 1.1-fold, at least about 2-fold, at least about 5-fold, at least about 10-fold, at least about 50-fold, at least about 100-fold, at least about 500-fold, at least about 1000-fold, at least about 5000-fold, at least about 10,000-fold, or at least about 100,000-fold compared to the cognate effector protein when assayed in a comparable fashion and/or via the same assay wherein the assay is an appropriate assay known in the art. In some embodiments, the engineered effector protein exhibits one or more improved characteristics compared to the cognate effector protein (e.g., a naturally occurring counterpart effector protein) when assayed via the same or a comparable assay known in the art. In some embodiments, improved characteristics are compared via one or more assays described herein, including assays described in the Examples. In some embodiments, the engineered polypeptide comprises at least two improved characteristics. In some embodiments, the engineered polypeptide comprises at least three improved characteristics. In some embodiments, the engineered polypeptide comprises only one improved characteristic. In some embodiments, the engineered polypeptide comprises only two improved characteristics.

In some embodiments, effector proteins described herein can be modified with the addition of one or more heterologous peptides or heterologous polypeptides (referred to collectively herein as a heterologous polypeptide). In some embodiments, an effector protein modified with the addition of one or more heterologous peptides or heterologous polypeptides may be referred to herein as a fusion protein. Such fusion proteins are described herein and throughout.

In some embodiments, the effector protein (e.g., polypeptide) described herein is fused to at least one heterologous polypeptide. In some embodiments, the effector protein (e.g., polypeptide) is fused to at least one heterologous polypeptide, and optionally wherein the at least one heterologous polypeptide comprises a nuclear localization signal (NLS).

In some embodiments, a heterologous peptide or heterologous polypeptide comprises a subcellular localization signal. In some embodiments, a subcellular localization signal can be a NLS. In some embodiments, a heterologous polypeptide comprises a NLS. In some embodiments, the NLS facilitates localization of a nucleic acid, protein, or small molecule to the nucleus, when present in a cell that contains a nuclear compartment. TABLE 2 lists exemplary NLS sequences. In some embodiments, the subcellular localization signal is a nuclear export signal (NES), a sequence to keep an effector protein retained in the cytoplasm, a mitochondrial localization signal for targeting to the mitochondria, a chloroplast localization signal for targeting to a chloroplast, an ER retention signal, and the like. In some embodiments, an effector protein described herein is not modified with a subcellular localization signal so that the polypeptide is not targeted to the nucleus, which can be advantageous depending on the circumstance (e.g., when the target nucleic acid is an RNA that is present in the cytosol).

In some embodiments, a heterologous peptide or heterologous polypeptide comprises a chloroplast transit peptide (CTP), also referred to as a chloroplast localization signal or a plastid transit peptide, which targets the effector protein to a chloroplast. In some embodiments, chromosomal transgenes from bacterial sources require a sequence encoding a CTP sequence fused to a sequence encoding an expressed protein (e.g., effector protein, fusion partner, or combinations thereof) if the expressed protein is to be compartmentalized in the plant plastid (e.g., chloroplast). In some embodiments, the CTP is removed in a processing step during translocation into the plastid. Accordingly, localization of an effector protein to a chloroplast is often accomplished by means of operably linking a polynucleotide sequence encoding a CTP sequence to the 5′ region of a polynucleotide encoding the exogenous protein.

In some embodiments, the heterologous polypeptide is an endosomal escape peptide (EEP). An EEP is an agent that quickly disrupts the endosome in order to minimize the amount of time that a delivered molecule, such an effector protein, spends in the endosome-like environment, and to avoid getting trapped in the endosomal vesicles and degraded in the lysosomal compartment. An exemplary EEP is set forth in TABLE 2.

In some embodiments, the heterologous polypeptide is a cell penetrating peptide (CPP), also known as a Protein Transduction Domain (PTD). A CPP or PTD is a polypeptide, polynucleotide, carbohydrate, or organic or inorganic compound that facilitates traversing a lipid bilayer, micelle, cell membrane, organelle membrane, or vesicle membrane.

Further suitable heterologous polypeptides include, but are not limited to, proteins (or fragments/domains thereof) that are boundary elements (e.g., CTCF), proteins and fragments thereof that provide periphery recruitment (e.g., Lamin A, Lamin B, etc.), and protein docking elements (e.g., FKBP/FRB, Pil1/Aby1, etc.).

In some embodiments, a heterologous peptide or heterologous polypeptide comprises a protein tag. In some embodiments, the protein tag is referred to as purification tag or a fluorescent protein. In some embodiments, the protein tag is detectable for use in detection of the effector protein and/or purification of the effector protein. Accordingly, in some embodiments, compositions, systems and methods comprise a protein tag or use thereof. In some embodiments, any suitable protein tag is used depending on the purpose of its use. Non-limiting examples of protein tags include a fluorescent protein, a histidine tag, e.g., a 6×His tag (SEQ ID NO: 113); a hemagglutinin (HA) tag; a FLAG tag; a Myc tag; and maltose binding protein (MBP). In some embodiments, the protein tag is a portion of MBP that can be detected and/or purified. Non-limiting examples of fluorescent proteins include green fluorescent protein (GFP), yellow fluorescent protein (YFP), red fluorescent protein (RFP), cyan fluorescent protein (CFP), mCherry, and tdTomato.

In some embodiments, a heterologous polypeptide is located at or near the amino terminus (N-terminus) of the effector protein disclosed herein. In some embodiments, a heterologous polypeptide is located at or near the carboxy terminus (C-terminus) of the effector proteins disclosed herein. In some embodiments, a heterologous polypeptide is located internally in an effector protein described herein (i.e., is not at the N- or C-terminus of an effector protein described herein) at a suitable insertion site.

In some embodiments, polypeptides (e.g., effector proteins or fusion proteins) described herein comprises 1, 2, 3, 4, 5, 6, 7, 8, 9, 10, or more heterologous polypeptides at or near the N-terminus, 1, 2, 3, 4, 5, 6, 7, 8, 9, 10, or more heterologous polypeptides at or near the C-terminus, or a combination of these (e.g., one or more heterologous polypeptides at the amino-terminus and one or more heterologous polypeptides at the carboxy terminus). In some embodiments, when more than one heterologous polypeptide is present, each are selected independently of the others, such that a single heterologous polypeptide is present in more than one copy and/or in combination with one or more other heterologous polypeptides present in one or more copies. In some embodiments, a heterologous polypeptide is considered near the N- or C-terminus when the nearest amino acid of the heterologous polypeptide is within about 1, 2, 3, 4, 5, 10, 15, 20, 25, 30, 40, 50, or more amino acids along the polypeptide chain from the N- or C-terminus.

In some embodiments, a heterologous polypeptide described herein comprises a heterologous polypeptide sequence recited in TABLE 2. In some embodiments, effector proteins described herein comprise an amino acid sequence that is at least about 65%, at least about 70%, at least about 75%, at least about 80%, at least about 85%, at least about 90%, at least about 95%, at least about 97%, at least about 98%, at least about 99%, or about 100% identical to any one of the amino acid sequences recited in TABLE 1 or TABLE 1.2 and further comprises one or more of the amino acid sequences set forth in TABLE 2. In some embodiments, a heterologous polypeptide described herein is a fusion partner as described en supra.

In some embodiments, polypeptides (e.g., effector proteins, fusion partners, fusion proteins, or combinations thereof) described herein are encoded by a codon optimized nucleic acid. In some embodiments, a nucleic acid sequence encoding an effector protein described herein, is codon optimized. In some embodiments, effector proteins described herein are codon optimized for expression in a specific cell, for example, a bacterial cell, a plant cell, a eukaryotic cell, an animal cell, a mammalian cell, or a human cell. In some embodiments, the effector protein is codon optimized for a human cell.

In some embodiments, polypeptides (e.g., effector proteins, fusion partners, fusion proteins, or combinations thereof) comprise one or more modifications that, in some embodiments, provide altered activity as compared to a naturally-occurring counterpart (e.g., a naturally-occurring nuclease, nickase, base editor, or deaminase activity which may be a naturally-occurring effector protein). In some embodiments, activity (e.g., nickase, nuclease, binding, base editing, or deaminase activity) of effector proteins described herein is measured relative to a naturally-occurring effector protein or compositions containing the same in a cleavage assay.

For example, in some embodiments, polypeptides (e.g., effector proteins, fusion partners, fusion proteins, or combinations thereof) comprise one or more modifications that provide increased activity (e.g., catalytic or binding activity) as compared to a naturally-occurring counterpart. In some embodiments, as another example, effector proteins provide increased catalytic activity (e.g., nickase, nuclease, binding, base editing, or deaminase activity) as compared to a naturally-occurring counterpart. In some embodiments, effector proteins provide enhanced nucleic acid binding activity (e.g., enhanced binding of a guide nucleic acid, and/or target nucleic acid) as compared to a naturally-occurring counterpart. In some embodiments, an effector protein comprises a 1%, 5%, 10%, 20%, 30%, 40%, 50%, 60%, 70%, 80%, 90%, 100%, 120%, 140%, 160%, 180%, 200%, or more, increase of the activity of a naturally-occurring counterpart.

Alternatively, or additionally, polypeptides (e.g., effector proteins, fusion partners, or combinations thereof) comprise one or more modifications that reduce the activity (e.g., catalytic (e.g., nickase, nuclease, base editing, or deaminase activity) or binding activity) of the polypeptides relative to a naturally occurring counterpart. In some embodiments, a polypeptide comprises a 100%, 90%, 80%, 70%, 60%, 50%, 40%, 30%, 20%, 10%, 5%, 1%, or less, decrease of the activity of a naturally occurring counterpart. In some embodiments, decreased activity comprises decreased catalytic activity (e.g., nickase, nuclease, binding, base editing, or deaminase activity) as compared to a naturally-occurring counterpart.

dCas Proteins

In some embodiments, an effector protein that has decreased catalytic activity is referred to as catalytically or enzymatically inactive, catalytically or enzymatically dead, as a dead protein or a dCas protein. In some embodiments, such a protein comprises an enzymatically inactive domain (e.g., inactive nuclease domain). For example, a nuclease domain (e.g., HEPN domain) of an effector protein, in some embodiments, is deleted or mutated relative to a wildtype counterpart so that it is no longer functional or comprises reduced nuclease activity. In some embodiments, a catalytically inactive effector protein binds to a guide nucleic acid and/or a target nucleic acid but does not cleave the target nucleic acid. In some embodiments, a catalytically inactive effector protein associates with a guide nucleic acid to activate or repress transcription of a target nucleic acid. In some embodiments, a catalytically inactive effector protein is fused to a fusion partner protein that confers an alternative activity to an effector protein activity. Such fusion proteins are described herein and throughout.

### Protein Engineering Methods

In some embodiments, effector proteins of the present disclosure are engineered, using any suitable protein engineering method known in the art. Examples of suitable protein engineering methods are described herein. In some embodiments, suitable protein engineering methods include a method of using mutagenesis to generate a novel nucleic acid encoding a novel effector protein or novel polypeptide, which novel effector protein is itself a modified biological molecule and/or contributes to the generation of another modified biological molecule as compared to wild-type equivalents. In some embodiments, protein engineering methods are geared towards maintaining certain existing protein functions while modifying others (e.g., maintaining binding activity to a guide nucleic acid, while modifying nuclease activity or specificity), increasing existing protein function, gaining a novel protein function, improving the stability of a protein under certain conditions, improving function in different environments, such as, for example, high temperature and/or high salt, or combinations thereof. In some embodiments, suitable protein engineering methods include, but are not limited to, random mutagenesis, focused mutagenesis, or methods that integrate both random and focused mutagenesis. In some embodiments, effector proteins are engineered in vitro or in vivo by eukaryotic cells or by prokaryotic cells.

Random mutagenesis engineering methods can generate random point mutations at codons corresponding to specific structurally characterized residues (e.g., protein residues involved in binding or catalysis, such as, for example, catalytic residues a HEPN nuclease active site). Although protein engineering by methods such as directed evolution via repeated random mutagenesis (e.g., random chemical or error prone (epPCR)) and selection can yield engineered proteins with desirable characteristics, some protein engineering efforts require more specificity. For example, in some embodiments, protein engineering methods which require mutation of more than one nucleotide relative to a non-modified codon require focused mutagenesis. In some embodiments, focused mutagenesis only introduces specific amino acid substitutions at positions corresponding to targeted nucleotide(s) or targeted residue(s). In some embodiments, focused mutagenesis employs a synthetic nucleic acid, such as a synthetic DNA oligonucleotide comprising one or more modifications. In some embodiments, a synthetic DNA oligonucleotide comprising one or more modifications is also be referred to as a mutagenic oligonucleotide. In some embodiments, the mutagenic oligonucleotide, is incorporated into a gene library as a mutagenic cassette. In some embodiments, the mutagenic oligonucleotide comprises modified/degenerate codons corresponding to targeted residues. In some embodiments, focused mutagenesis also yields more functional variations, beneficial mutations, or modifications resulting in the desired engineered protein activity while minimizing neutral or deleterious mutations.

In some embodiments, effector proteins are engineered in vitro or in vivo by focused and/or random mutagenesis methods, such as chemical mutagenesis, combinatorial libraries, computational strategies for high-quality library design, homologous recombination, non-homologous recombination, recombination based methods such as DNA shuffling (i.e., molecular breeding), directed evolution, deletion mutagenesis, error prone PCR (epPCR), insertion mutagenesis, random mutagenesis, scanning mutagenesis, site-directed mutagenesis (SDM) (and similar methods such as site-specific mutagenesis, oligonucleotide-directed mutagenesis, site-saturation mutagenesis (SSM)), use of mutator strain, assembly PCR, sexual PCR mutagenesis, cassette mutagenesis, recursive ensemble mutagenesis, exponential ensemble mutagenesis, site-specific mutagenesis, gene reassembly, gene site saturation mutagenesis (GSSM), synthetic ligation reassembly (SLR), recombination, replacing codon(s) encoding the same amino acid, recursive sequence recombination, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deletion mutagenesis, restriction-selection mutagenesis, restriction-purification mutagenesis, artificial gene synthesis, ensemble mutagenesis, chimeric nucleic acid multimer creation, other mutagenesis methods described herein, or combinations thereof (see, e.g., Packer, M., et al., Nature Reviews Genetics, 16(7):379-94 (2015)).

In some embodiments, in vivo mutagenesis methods are focused, random, or combinations thereof. In some embodiments, in vivo focused mutagenesis methods comprise selectively introducing localized DNA damage into a genome, such as, for example, targeting a pathway requiring long-range resection so as to form a single-stranded region during biasing repair and selectively mutate said single-stranded region. In some embodiments, in vivo focused mutagenesis methods comprise delivering a nucleic acid encoding an effector protein and a guide nucleic acid to a cell, and contacting the cell with a mutator compound or mutator enzyme. In some embodiments, in vivo focused mutagenesis methods comprise selectively introducing localized DNA damage in a preselected region of an organism's DNA in vivo, biasing repair of the localized DNA damage by targeting a pathway requiring long-range resectioning of the localized DNA damage, wherein the DNA forms a single-stranded region during the biasing repair, and selectively mutating the single stranded region to cause targeted mutagenesis, optionally wherein the organism is an eukaryotic organism. In some embodiments, localized DNA damage is a double stranded break (e.g., DSB). In some embodiments, a DSB is introduced by a DNA mutator enzyme domain (e.g., DNA glycosylase, 3-methyladenine glycosylase Ma lp (e.g., Maglp), DNA nuclease, Fokl). In some embodiments, biasing repair of the DSB involves contacting the cell with a compound that elicits DNA damage checkpoint activation. In some embodiments, the compound that elicits DNA damage checkpoint activation is a chemical checkpoint activator (MMS, enzymatic checkpoint activator, Magi). In some embodiments, in vivo random mutagenesis methods (i.e., traditional genetic screens) randomly damage DNA via chemical and/or physical agents such as, for example, alkylating compounds (e.g., ethyl methanesulfonate (EMS)), deaminating compounds (e.g., nitrous acid), base analogues (e.g., 2-aminopurine), radiation (e.g., ultraviolet irradiation), bisulfite, or combinations thereof. In some embodiments, random chemical mutagenesis facilitates dose-dependent modification or mutation of DNA. In some embodiments, random chemical mutagenesis, which has a broad mutational spectrum, is used to randomly deactivate genes for a genome-wide screen in vivo or in vitro. In some embodiments, random mutagenesis enhances the error rate during DNA replication, which leads to off-target mutations and/or deleterious genome mutations.

In some embodiments, random mutator strain mutagenesis, an in vivo random mutagenesis method, produces randomly mutagenized plasmid libraries upon propagation of the genes cloned in plasmids through a mutator strain, like Escherichia coli XL1-red. In brief, random mutator strain mutagenesis is a method for introducing random point mutations throughout a gene encoding a protein of interest with the use of a plasmid. The method involves transformation and propagation of a plasmid containing the target gene into a mutator strain, isolating the resulting randomly mutagenized plasmid library, transforming the library into a strain comprising the mutant target gene, and screening the mutant target gene phenotype. In some embodiments, the method elicits random mutagenesis via phage-assisted continuous evolution (PACE), a method which harnesses the phage virus bacterial infection cycle to generate multiple rounds of DNA sequence mutations, selecting for DNA mutations in a mutant target gene encoding a protein that result in a desired protein structure or activity. In some embodiments, random mutagenesis involves yeast orthogonal replication. Although the methods generally offer ease of use, in some embodiments, host intolerance to a high degree of genomic mutation(s) places an upper limit on in vivo mutagenesis rates. In vitro random mutagenesis methods generally offer protein engineering methods with higher target mutation rates as compared to most in vivo random mutagenesis methods.

In some embodiments, homologous recombination, a random mutagenesis method which can be carried out in vivo or in vitro, leads to DNA modification, damage, or repair upon DNA shuffling, family shuffling, staggered extension process (StEP), random chimeragenesis on transient templates (RACHITT), nucleotide exchange and excision technology (NexT), heritable recombination, assembly of designed oligonucleotides (ADO), synthetic shuffling, or combinations thereof. For example, StEP is a modified PCR that uses highly abbreviated annealing and extension steps to generate staggered DNA fragments and promote crossover events along the full length of the template sequence(s), such that most of the resulting polypeptides comprise sequence information from different template sequence(s). RACHITT performs molecular mutagenesis at a high recombination rate by aligning parental gene fragments on a full-length DNA template, which are then stabilized on the template by a single long annealing step at a relatively high ionic strength. In some embodiments, RACHITT yields a considerable number of crossovers per gene in a single annealing step. NexT is also a modified PCR that uses uridine triphosphate (dUTP) as a DNA fragmentation defining exchange nucleotide with thymidine. In NexT, the exchange nucleotides are removed enzymatically, followed by chemical cleavage of the DNA backbone. Finally, the oligonucleotide pool is reassembled into full-length genes by internal primer extension, and the recombined gene library is amplified by standard PCR. Another modified PCR, ADO, is a two-step reaction involving an overlap extension PCR step using synthetic oligonucleotides followed by a PCR amplification step using outer primers, resulting in double-stranded DNA assembled with engineered gene fragments. In some embodiments, homologous recombination (HR) methods lead to DNA modification comprising knocking out, or removing, mutations. In some embodiments, HR methods repair gene function by identifying sequence homology and replicating the functional version of the target gene. In some embodiments, knock out mutations result in functional modifications to the protein encoded by the modified nucleic acid sequence. In some embodiments, HR proves advantageous in its ability to identify beneficial mutation combinations, eliminate passenger mutations, shuffle functional sequences of orthologous proteins, or combinations thereof.

In some embodiments, error prone PCR (epPCR) mutagenesis, an in vitro random mutagenesis method, results in the modification/damage of DNA via PCR amplification involving supplemental mixture components such as, for example, proprietary enzyme mixes (e.g., Mutazyme), Taq supplemented with Mg2+, Taq supplemented with Mn2+ and/or unequal dNTPs, or combinations thereof. EpPCR involves the modification of DNA or creation of a mutation during PCR amplification of a target gene, a fragment of a target gene, a target sequence, a DNA sequence, or combinations thereof. In some embodiments, the low fidelity of DNA polymerases under certain conditions generates point mutations during PCR amplification of a gene of interest. In some embodiments, the base-pairing fidelity of DNA polymerases can be reduced with increased magnesium concentrations (e.g., Taq supplemented with Mg2+), supplementation with manganese (e.g., Taq supplemented with Mn2+), the use of mutagenic dNTP analogues (e.g., unequal/unbalanced dNTPs), or the use of proprietary enzyme mixes (e.g., Mutazyme) to increase mutation rates (e.g., 10⁻⁴˜10⁻³ per replicated base). Given that each cycle of PCR amplification leads to the accumulation of mutations, high mutation rates (e.g., high number mutations per clone) can be achieved by increasing the number of PCR amplification cycles. EpPCR offers advantages, such as, for example, its tendency for high mutation rates and/or a relatively even mutation spectrum, as well as easy to use commercial formulations. Optionally, in some embodiments, a more ideal nucleotide mutational spectrum is achieved via sequence saturation mutagenesis (SeSaM), a mutagenesis method that randomizes a target sequence at every single nucleotide position. Briefly, SeSaM is a chemo-enzymatic random mutagenesis method which involves the enzymatic insertion of a base, such as the universal base deoxyinosine (2′-deoxyInosine (dI)), throughout the target gene.

Suitable applications of epPCR include, but are not limited to, the generation of neutral drift libraries, which, in some embodiments, are used to identify an evolvable starting point for protein engineering (e.g., the directed evolution of a target protein of interest). In some embodiments, generating a neutral drift library involves exploring accessible sequence space by repeated rounds of mutagenesis and selection for the accumulation of mutations that are largely neutral and compatible with maintaining wild-type function. Mutations that are largely neutral for the wild-type protein function accumulate, while mutations detrimental to the wild-type protein function are purged, yielding a library of high diversity and quality. Specifically, a target gene is mutagenized by epPCR, fused to a reporter nucleic acid (e.g., GFP reporter), and the mutagenized gene variants are then screened for target protein expression. After multiple rounds of mutagenesis and screening, the resulting neutral drift library exhibits sequence diversity that does not destabilize protein structure or protein function. Screening for target protein expression ensures the resulting neutral drift library mostly lacks non-target deleterious mutations.

Another in vitro method for generating high-quality libraries is site-directed saturation mutagenesis (SDSM). SDSM and similar methods such as site-directed mutagenesis (SDM), site-saturation mutagenesis (SSM), site-specific mutagenesis, or oligonucleotide-directed mutagenesis, are in vitro focused mutagenesis methods, capable fully sampling the amino acid repertoire, and/or focusing on functionally relevant residues, increasing library quality. In some embodiments, SDSM involves NNK and NNS codons (where N can be any of the four nucleotides, K can be G or T, and S can be G or C) on mutagenic primers. SDM, which is commonly applied to study the function of a single amino acid in relation to the rest of the protein, involves the substitution of a single amino acid is substituted for another, usually an alanine. In some embodiments, site-directed mutagenesis is performed via means that are synthetic, where the design of the engineered/desirable/target/progeny polynucleotide(s) is derived by analysis of a wild-type/parental set of proteins and/or of the polypeptides correspondingly encoded by the wild-type/parental proteins. SSM, which is a similar is a similar method to SDM, involves the substitution of a single amino acid is substituted for another, usually for any of the other 19 possible substituents. Thus, the SSM mutagenesis product is a collection of clones, each having a different codon in the targeted position (i.e., saturated), yielding all possible substitutions. Analysis of the SSM mutagenesis product can indicate the relationship between the targeted amino acid positions and protein function. In some embodiments, site-specific protein engineering methods, such as SSM, target the diversification of functionally relevant residues, some of which may not be comprised in the protein's primary structure. In some embodiments, simultaneous SSM of, for example, multiple target residues, can result in combinations of mutations that, in some embodiments, exhibit synergistic or epistatic interactions. Combinations of mutations exhibiting epistatic interactions (e.g., sign epistasis, a type of interaction in which mutations may be individually non-desirable/deleterious, but confer gain-of-function in combination) can be selected for with the use of simultaneous SSM. In some embodiments, simultaneous SSM targets combinations of mutations exhibiting synergistic interactions (e.g., a type of interaction in which mutations in combination have a greater effect as compared to the sum of the effects of each individual mutation) with desirable/target effects. Overall, in some embodiments, a site-saturation library results from sequential enrichment of epistatic mutation combinations, sequential enrichment of synergistic mutation combinations, sequential enrichment of functionally relevant mutations, sequential enrichment of functionally relevant residues, or combinations thereof. Site-specific mutagenesis or oligonucleotide-directed mutagenesis involves the modification of DNA or creation of an intentional mutation at a specific location on the oligonucleotide sequence. In some embodiments, modification of DNA or creation of an intentional mutation involves insertional mutagenesis and/or deletion mutagenesis. In some embodiments, insertional mutagenesis involves the incorporation of a mutation into a target gene via the incorporation of a few nucleotides (e.g., insertional mutagenesis via conventional PCR, nested PCR, or similar techniques). In some embodiments, deletion mutagenesis involves the removal of a target gene, a fragment of a target gene, a target sequence, a DNA sequence, a few nucleotides, or combinations thereof (e.g., deletion mutagenesis via inverse PCR, or a similar technique). In some embodiments, site-specific mutagenesis or oligonucleotide-directed mutagenesis involves amplifying a gene of interest via PCR with the use of a synthetic primer possessing a specific mutation or a target mutation, which, in some embodiments, results in a deletion, insertion, or single nucleotide polymorphism (SNP), as confirmed by sequencing. In some embodiments, oligonucleotide-directed mutagenesis involves the replacement of a short sequence with a synthetically mutagenized oligonucleotide. In brief, a synthetically mutagenized oligonucleotide, in some embodiments, comprises one or more modifications, such as, for example, modified codon(s) corresponding to targeted residue(s). Mutagenesis with synthetic oligonucleotides requires sequencing of individual clones after each selection round, grouping individual clones into families, arbitrarily choosing a single family, and reducing the chosen family to a consensus motif. The consensus motif is resynthesized and reinserted into a single gene for additional selection. In some embodiments, oligonucleotide-directed mutagenesis is best suited for fine-tuning sequence areas of comparatively low information content. Cassette mutagenesis, a type of SDM, uses a short, double-stranded oligonucleotide sequence (i.e., a gene cassette) to replace a fragment of target DNA such that, a sequence block of a single template is typically replaced by a (partially) randomized sequence (e.g., a mutagenic cassette, which may be a mutagenic oligonucleotide).

In some embodiments, computational strategies, an in vitro focused mutagenesis method for high-quality library design, involves Rosetta design, computationally guided libraries, incorporating synthetic oligonucleotides via gene reassembly (ISOR), consensus design, reconstructed evolutionary adaptive path (REAP) analysis, and SCHEMA algorithm(s). The method offers an advantage in the form of creating small libraries pre-enriched for functional variation by natural selection and/or in silico filtering. In some embodiments, consensus design (a method which involves the identification of common ancestral mutations (i.e., evolutionary history) by aligning all sequences and identifying the most frequently observed amino acid(s) at each position in the sequence alignment) leads to the introduction of consensus mutations or significantly distinct/divergent mutations, yielding engineered proteins with improved thermostability, catalytic stability, enzymatic efficiency, or combinations thereof. In contrast, reconstructed evolutionary adaptive path (REAP) analysis provides a method for the identification of significant mutational divergence, which, in some embodiments, (i) comprises mutational signatures related to known protein function(s) or protein pathway characteristics, or which, in some embodiments, is (ii) used to predict changes in protein function(s) as related to, for example, structural proximity to an active site. In some embodiments, a protein engineering method, incorporating synthetic oligonucleotides via gene reassembly (ISOR), is used to predict desirable protein engineering outcomes, such as, for example, the introduction of mutations that, in some embodiments, improve protein stability and/or protein folding. ISOR, a versatile combinatorial method for the partial diversification of large sets of protein residues or targeted protein positions, offers a method to select target engineered proteins capable of desirable/target activity/properties. As compared to site-specific methods of diversification, in some embodiments, ISOR proves more efficient in identifying target protein positions related to target protein activity, while building a reasonably sized protein library for protein engineering. Briefly, ISOR incorporates synthetic oligonucleotides comprising randomized codons flanked by wild-type sequences to wild-type gene fragments via assembly PCR. The resulting reassembled gene comprises randomized cassettes (e.g., mutagenic cassettes) at target sites. As a factor of oligonucleotide concentration, the resulting reassembled gene comprises semi-randomly introduced mutations, such that, in some embodiments, resulting variants comprise a different quantity and/or combination of mutated positions. In some embodiments, randomly introduced mutations comprise a random subset of the resulting mutations. In some embodiments, ISOR is used to create libraries focused on the randomization of individual positions of interest, on the identification of proteins comprising combinations of mutated residues while maintaining/upregulating/downregulating wild-type protein function, and/or on the identification of proteins comprising combinations of mutated residues while gaining a desirable protein function. In some embodiments, ISOR is used to create libraries characterizing protein function as related to insertions and/or deletions in sequence positions surrounding an active site of interest.

In some embodiments, computational strategies or computational modelling, as described herein, facilitate the identification of specific amino acid substitution/modification as related to desired/target engineered protein activity/function. In some embodiments, computational strategies for high-quality library design, involve, for example, the use of computational algorithms such as SCHEMA and/or Rosetta. Briefly, SCHEMA provides a method for identifying protein fragments and designing novel proteins by recombination of homologous sequences. For example, SCHEMA identifies interacting amino acid residue pairs via structural information, accounting for amino acid residue pair interactions that are broken upon recombination, and predicting which elements in homologous sequences/proteins can be swapped without disturbing the integrity of the protein structure. Briefly, Rosetta is a computational modeling software comprising algorithms which, in some embodiments, is used to design methods for protein engineering based on protein structure analysis, such as, for example, protein structure prediction, protein structure refinement, protein conformation, protein docking, functional protein design, and combinations thereof. In some embodiments, rosetta models are employed to adapt protein engineering methods to specific applications, such as, for example, protein-protein docking interaction/activity of engineered protein(s). In some embodiments, rosetta models are also employed to consider protein folding, translation, rotation, association, amino acid sequence design, molecular structure interactions, degrees of freedom (DOFs), electrostatic interactions, hydrogen bonding, hydrophobic interactions, electrostatic interactions, or combinations thereof. In some embodiments, Rosetta models facilitate the design of a protein engineering method to optimize protein sequences (including, for instance, suggesting a single base change) for engineering protein(s) capable of a target protein conformation. In some embodiments, Rosetta models are geared towards maintaining existing protein function, increasing existing protein function, gaining a novel protein function, improving the stability of protein function, improving function in different environments, such as, for example, high temperature and/or high salt, or combinations thereof. In some embodiments, Rosetta's design models are employed to identify mutations that improve engineered protein stability and binding affinity.

In some embodiments, non-homologous recombination is an in vitro focused mutagenesis method which leads to DNA modification, damage, or repair upon incremental truncation for the creation of hybrid enzymes (ITCHY), sequence homology-independent protein recombination (SHIPREC), nonhomologous random recombination (NRR), sequence-independent site-directed chimeragenesis (SISDC) and overlap extension PCR. For example, ITCHY is a recombination method capable of generating a single-crossover hybrid library based on generation of N- or C-terminal fragment libraries of two genes by progressive truncation of the coding sequences by an exonuclease followed by ligation. Thus, ITCHY allows the creation of hybrid libraries between fragments of genes without any sequence dependency. SHIPREC is a recombination method capable of generating single-crossover hybrid libraries of unrelated or distantly related proteins by maintaining sequence alignment between the parent sequences and introducing crossovers mainly at structurally related sites distributed over the aligned sequences. NRR is a recombination method that enables nucleic acid or DNA fragments to randomly recombine in a length-controlled manner at sites where there is little or no sequence homology. SISDC is a recombination method that enables the recombination of distantly related (or unrelated) proteins at multiple discrete sites, such as sites related to protein function. In some embodiments, non-homologous recombination (NHR) leads to the recombination of portions of nucleic acid(s) at sites with low or no sequence homology. Thus, in some embodiments, NHR increases the frequency at which novel modified nucleic acid sequences are generated, yielding a more efficient and/or complete exploration of nucleic acid or protein diversity, as compared to HR. In some embodiments, NHR proves advantageous in its capacity to shuffle distantly related sequences, rearrange gene order, rearrange nucleic acids comprising low information content, or combinations thereof.

In some embodiments, the methods for protein engineering comprise generating a nucleic acid encoding a polypeptide comprising a mutation or modification (e.g., deleting or adding one or more nucleotides, or a combination thereof) wherein the methods for introducing the mutation or modification comprise any of the protein engineering methods disclosed herein. In some embodiments, the method for protein engineering further comprising expressing nucleic acid comprising a mutation or modification to generate a polypeptide comprising a mutation or modification. In some embodiments, the methods described herein comprise repeating the method for protein engineering until the desired modification or mutation is achieved.

In some embodiments, the methods for protein engineering further comprise a screening step, an assaying step, an isolation step, a purification step, or combinations thereof. In some embodiments, the engineered effector proteins are further processed by unfolding (e.g., heat denaturation, dithiothreitol reduction, etc.) and may be further refolded, using any suitable method.

### Fusion Proteins

In some embodiments, compositions, systems, devices, kits, and methods comprise an effector partner or use thereof. In some embodiments, when an effector partner is provided herein, reference is made to a protein, polypeptide or peptide that can, in combination with an effector protein, impart some function or activity that can be used to effectuate modification(s) of a target nucleic acid described herein and/or change expression of the target nucleic acid or other nucleic acids associated with the target nucleic acid, when used in connection with compositions, systems, and methods described herein. Examples of an effector partner provided herein include fusion partners as described herein. It is understood that when referring to an effector partner herein reference is also made to a fusion partner and vice versa. Fusion partners and fusion proteins thereof are further described in detail throughout the present disclosure.

In some embodiments, compositions, systems, devices, kits, and methods comprise a fusion protein or uses thereof. The fusion protein generally comprises at least one effector protein and at least one fusion partner protein. In some embodiments, the fusion partner comprises a polypeptide or peptide that is fused or linked to the effector protein. In some embodiments, the fusion partner protein is fused to the N-terminus of the effector protein. In some embodiments, the fusion partner protein is fused to the C-terminus of the effector protein. In some embodiments, the terms fusion partners and fusion partner proteins are used interchangeably herein.

In some embodiments, the effector partner (e.g., fusion partner) is a heterologous peptide or polypeptide as described herein. In some embodiments, the fusion partner is not an effector protein as described herein. In some embodiments, the fusion partner comprises a second effector protein or a multimeric form thereof. In some embodiments, the fusion protein is a multimeric protein. In some embodiments, the multimeric protein is a homomeric protein. In some embodiments, the multimeric protein is a heteromeric protein. Accordingly, in some embodiments, the fusion protein comprises more than one effector protein. In such embodiments, the fusion protein can comprise at least two effector proteins that are same. In some embodiments, the fusion protein comprises at least two effector proteins that are different. In some embodiments, the multimeric form is a homomeric form. In some embodiments, the multimeric form is a heteromeric form. Unless otherwise indicated, reference to effector proteins throughout the present disclosure include fusion proteins comprising the effector protein described herein and a fusion partner.

In some embodiments, the fusion partner is a heterologous protein that imparts some function or activity that is not provided by an effector protein. In some embodiments, the fusion partner modifies (e.g., edits, cleaves) a nucleic acid (e.g., target nucleic acid or non-target nucleic acid).

In some embodiments, the fusion protein disclosed herein provides cleavage activity, such as cis cleavage activity, trans cleavage activity, nickase activity, nuclease activity, other activity, or a combination thereof. In some embodiments, fusion proteins disclosed herein comprise a HEPN domain comprising cleavage activity. In some embodiments, fusion proteins disclosed herein cleave nucleic acids, including single stranded RNA (ssRNA) and double stranded RNA (dsRNA). In some embodiments, fusion proteins cleave the target nucleic acid at the target sequence or adjacent to the target sequence. In some embodiments, fusion proteins cleave the non-target nucleic acid.

In some embodiments, the fusion protein complexes with a guide nucleic acid and the complex interacts with the target nucleic acid, a non-target nucleic acid, or both. In some embodiments, the interaction comprises one or more of: recognition of the target nucleic acid by the effector protein, hybridization of the guide nucleic acid to the target nucleic acid, modification of the target nucleic acid and/or the non-target nucleic acid by the fusion protein, or combinations thereof. In some embodiments, recognition of the target nucleic acid directs the modification activity of the fusion protein.

In some embodiments, modification activity of the fusion protein described herein comprises cleavage activity, binding activity, insertion activity, and substitution activity. In some embodiments, modification activity of an effector protein results in: cleavage of at least one strand of a target nucleic acid, deletion of one or more nucleotides of a target nucleic acid, insertion of one or more nucleotides into a target nucleic acid, substitution of one or more nucleotides of a target nucleic acid with an alternative nucleotide, more than one of the foregoing, or any combination thereof. In some embodiments, the ability of the fusion protein to modify a target nucleic acid depends upon the effector protein being complexed with a guide nucleic acid, the guide nucleic acid being hybridized to a target sequence of the target nucleic acid, or combinations thereof. In some embodiments, a target nucleic acid comprises a target strand and a non-target strand. Accordingly, in some embodiments, the fusion protein modifies a target strand and/or a non-target strand of a target nucleic acid.

In some embodiments, the fusion protein described herein comprises a heterologous amino acid sequence that affects formation of a multimeric complex of the fusion protein. By way of non-limiting example, the fusion protein comprises an effector protein described herein and a fusion partner comprising a Calcineurin A tag, wherein the fusion protein dimerizes in the presence of Tacrolimus (FK506). Also, by way of non-limiting example, the fusion protein comprises an effector protein described herein and a SpyTag configured to dimerize or associate with another effector protein in a multimeric complex. Multimeric complex formation is further described herein.

In some embodiments, the effector partner (e.g., fusion partner) imparts a function or activity to the fusion protein comprising an effector protein that is not provided by the effector protein, including but are not limited to: nuclease activity, methyltransferase activity, demethylase activity, DNA repair activity, DNA damage activity, RNA repair activity, RNA damage activity, deamination activity, dismutase activity, alkylation activity, depurination activity, oxidation activity, dimer forming activity (e.g., pyrimidine dimer forming activity), integrase activity, transposase activity, recombinase activity, polymerase activity, ligase activity, helicase activity, photolyase activity, glycosylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity, modification of a polypeptide associated with target nucleic acid (e.g., a histone), and/or signaling activity. In some embodiments, the fusion partner provides signaling activity. In some embodiments, the fusion partner inhibits or promotes the formation of multimeric or multiprotein complex of an effector protein.

In some embodiments, the fusion partner directly or indirectly modifies a target nucleic acid. Modifications can be of a nucleobase, nucleotide, or nucleotide sequence of a target nucleic acid. In some embodiments, the fusion partner interacts with additional proteins, or functional fragments thereof, to make modifications to a target nucleic acid. In other embodiments, the fusion partner modifies proteins associated with a target nucleic acid. In some embodiments, a fusion partner modulates transcription (e.g., inhibits transcription, increases transcription) of a target nucleic acid. In yet another example, a fusion partner directly or indirectly inhibits, reduces, activates or increases expression of a target nucleic acid.

### Multiprotein Complex Formation Modification Activity

In some embodiments, an effector partner (e.g., fusion partner) inhibits the formation of a multiprotein complex of an effector protein. Alternatively, the effector partner (e.g., fusion partner) promotes the formation of a multiprotein complex of the effector protein.

By way of non-limiting example, in some embodiments, the fusion protein comprises an effector protein described herein and a fusion partner comprising a Calcineurin A tag, wherein the fusion protein dimerizes in the presence of Tacrolimus (FK506). Also, by way of non-limiting example, in some embodiments, the fusion protein comprises an effector protein described herein and a SpyTag configured to dimerize or associate with another effector protein in a multiprotein complex. Multiprotein complex formation is further described herein.

### Base Editing Enzymes

In some embodiments, effector partners (e.g., fusion partners) modify a nucleobase of a target nucleic acid. In some embodiments, fusion proteins comprising such a fusion partner and an effector protein are referred to as base editors. In some embodiments, the a fusion partner is referred to as a base editing enzyme. In some embodiments, a base editing enzyme variant that differs from a naturally occurring base editing enzyme, but it is understood that any reference to a base editing enzyme herein also refers to a base editing enzyme variant.

In some embodiments, a base editor is a system comprising an effector protein and a base editing enzyme. In some embodiments, the base editor comprises a base editing enzyme and an effector protein as independent components. In some embodiments, the base editor comprises a fusion protein comprising a base editing enzyme fused or linked to an effector protein. In some embodiments, the amino terminus of the fusion partner protein is linked to the carboxy terminus of the effector protein by the linker. In some embodiments, the carboxy terminus of the fusion partner protein is linked to the amino terminus of the effector protein by the linker. In some embodiments, the base editor is functional when the effector protein is coupled to a guide nucleic acid. In some embodiments, the base editor is functional when the effector protein is coupled to a target nucleic acid. In some embodiments, the base editor is functional when the effector protein complexed with a guide nucleic acid is hybridized to a target nucleic acid. In some embodiments, the guide nucleic acid imparts sequence specific activity to the base editor. By way of non-limiting example, the effector protein comprises a catalytically inactive effector protein (e.g., a catalytically inactive variant of an effector protein described herein). Also, by way of non-limiting example, the base editing enzyme comprises deaminase activity. Additional base editors are described herein.

In some embodiments, base editing enzymes or base editors catalyze editing (e.g., a chemical modification) of a nucleobase of a nucleic acid molecule, such as RNA (single stranded or double stranded). In some embodiments, a base editing enzyme, and therefore a base editor, is capable of converting an existing nucleobase to a different nucleobase, such as: an adenine (A) to guanine (G); cytosine (C) to uracil (U); cytosine (C) to guanine (G); uracil (U) to cytosine (C); guanine (G) to adenine (A); hydrolytic deamination of an adenine or adenosine, or methylation of cytosine (e.g., CpG, CpA, CpT or CpC). In the context of base editing, a person skilled in the art would recognize that reference to the nucleobase (e.g., adenine) or nucleotide (e.g., adenosine) that is being modified by the base editor or base editing enzyme is the nucleobase of the molecule. Accordingly, in the context of base editing, reference to a nucleobase and nucleotide are used interchangeably. In some embodiments, base editing enzymes edit a nucleobase on a ssRNA. In some embodiments, base editing enzymes edit a nucleobase on both strands of dsRNA. In some embodiments, base editing enzymes edit a nucleobase of an RNA.

In some embodiments, a base editing enzyme itself binds or does not bind to the nucleic acid molecule containing the nucleobase. In some embodiments, upon binding to its target locus in the target nucleic acid (e.g., a RNA molecule), base pairing between the guide nucleic acid and target strand leads to displacement of a small segment of ssRNA in a “stem-loop”. In some embodiments, RNA bases within the stem-loop are edited by the base editing enzyme or base editor having the deaminase enzyme activity.

In some embodiments, a base editing enzyme comprises a deaminase enzyme. Exemplary deaminases are described in US20210198330, WO2021041945, WO2021050571A1, and WO2020123887, all of which are incorporated herein by reference in their entirety. Exemplary deaminase domains are described WO 2018027078 and WO2017070632, and each are hereby incorporated in its entirety by reference. Also, additional exemplary deaminase domains are described in Komor et al., Nature, 533, 420-424 (2016); Gaudelli et al., Nature, 551, 464-471 (2017); Komor et al., Science Advances, 3:eaao4774 (2017), and Rees et al., Nat Rev Genet. 2018 December; 19(12):770-788. doi: 10.1038/s41576-018-0059-1, which are hereby incorporated by reference in their entirety. In some embodiments, the deaminase functions as a monomer. In some embodiments, the deaminase functions as heterodimer with an additional protein. In some embodiments, the fusion partner is a deaminase, e.g., ADAR1/2, ADAR-2, AID, or any functional variant thereof.

In some embodiments, a base editor described herein comprising one or more base editing enzymes (e.g., APOBEC1, nickase, and UGI) that efficiently edits in mammalian cells, while minimizing frequency of non-target indels. In some embodiments, base editors do not comprise a functional fragment of the base editing enzyme.

In some embodiments, the base editor is a cytosine base editor, wherein the base editing enzyme is a cytosine base editing enzyme. In some embodiments, the cytosine base editing enzyme is a cytidine deaminase. In some embodiments, the base editor comprising the cytidine deaminase is generated by ancestral sequence reconstruction as described in WO2019226953, which is hereby incorporated by reference in its entirety. Non-limiting exemplary cytidine deaminases suitable for use with effector proteins described herein include: APOBEC1, APOBEC2, APOBEC3C, APOBEC3D, APOBEC3F, APOBEC3G, APOBEC3H, APOBEC4, APOBEC3A, BE1 (APOBEC1-XTEN-dCas9), BE2 (APOBEC1-XTEN-dCas9-UGI), BE3 (APOBEC1-XTEN-dCas9(A840H)-UGI), BE3-Gam, saBE3, saBE4-Gam, BE4, BE4-Gam, saBE4, and saBE4-Gam as described in WO2021163587, WO2021087246, WO2021062227, and WO2020123887, which are incorporated herein by reference in their entirety.

In some embodiments, a base editor is a cytosine to guanine base editor (CGBE), wherein the base editing enzyme is a cytosine to guanine base editing enzyme. In some embodiments, the CGBE, converts a cytosine into a guanine.

In some embodiments, the base editor comprises an adenine deaminase (e.g., TadA). In some embodiments, the adenosine deaminase is a TadA monomer (e.g., Tad*7.10, TadA*8 or TadA*9). In some embodiments, the adenosine deaminase is a TadA*8 variant (e.g., any one of TadA*8.1, TadA*8.2, TadA*8.3, TadA*8.4, TadA*8.5, TadA*8.6, TadA*8.7, TadA*8.8, TadA*8.9, TadA*8.10, TadA*8.11, TadA*8.12, TadA*8.13, TadA*8.14, TadA*8.15, TadA*8.16, TadA*8.17, TadA*8.18, TadA*8.19, TadA*8.20, TadA*8.21, TadA*8.22, TadA*8.23, or TadA*8.24 as described in WO2021163587 and WO2021050571, which are each hereby incorporated by reference in its entirety). In some embodiments, the base editor comprises TadA.

In some embodiments, a base editing enzyme is a deaminase dimer. In some embodiments, the ABE comprises the effector protein, the adenine base editing enzyme and the deaminase dimer. In some embodiments, the deaminase dimer comprises an adenosine deaminase. In some embodiments, the deaminase dimer comprises TadA and a suitable adenine base editing enzyme including an: APOBEC3A, Anc APOBEC (a.k.a. AncBE4Max), BtAPOBEC2, and variants thereof. In some embodiments, the adenine base editing enzyme is fused to amino-terminus or the carboxy-terminus of TadA.

In some embodiments, a base editor is an RNA base editor, wherein the base editing enzyme is an RNA base editing enzyme. In some embodiments, the RNA base editing enzyme comprises an adenosine deaminase. In some embodiments, ADAR proteins bind to RNAs and alter their sequence by changing an adenosine into an inosine. In some embodiments, RNA base editors comprise an effector protein that is activated by or binds RNA.

In some embodiments, base editing enzymes, and therefore base editors, are used for treating a subject having or a subject suspected of having a disease related to a gene of interest. In some embodiments, base editing enzymes, and therefore base editors, are useful for treating a disease or a disorder caused by a point mutation in a gene of interest. In some embodiments, compositions, systems, and methods described herein comprise a base editor and a guide nucleic acid, wherein the base editor comprises an effector protein and a base editing enzyme, and wherein the guide nucleic acid directs the base editor to a sequence in a target gene.

### Linkers for Peptides

In some embodiments, a linker comprises a bond or molecule that links a first polypeptide to a second polypeptide. Accordingly, in some embodiments, effector proteins, fusion partners, or combinations thereof are connected by a linker. In some embodiments, the linker comprises or consists of a covalent bond. In some embodiments, the linker comprises or consists of a chemical group. In some embodiments, the linker comprises an amino acid. In some embodiments, a peptide linker comprises at least two amino acids linked by an amide bond. In general, the linker connects a terminus of the effector protein to a terminus of the fusion partner. In some embodiments, carboxy terminus of the effector protein is linked to the amino terminus of the fusion partner. In some embodiments, carboxy terminus of the fusion partner is linked to the amino terminus of the effector protein. In some embodiments, the effector protein and the fusion partner are directly linked by a covalent bond.

In some embodiments, linkers comprise one or more amino acids. In some embodiments, linker is a protein. In some embodiments, a terminus of the effector protein is linked to a terminus of the fusion partner through an amide bond. In some embodiments, a terminus of the effector protein is linked to a terminus of the fusion partner through a peptide bond. In some embodiments, linkers comprise an amino acid. In some embodiments, linkers comprise a peptide. In some embodiments, an effector protein is coupled to a fusion partner by a linker protein. In some embodiments, the linker comprises any of a variety of amino acid sequences. In some embodiments, the linker comprises a region of rigidity (e.g., beta sheet, alpha helix), a region of flexibility, or any combination thereof. In some embodiments, the linker comprises small amino acids, such as glycine and alanine, that impart high degrees of flexibility. The ordinarily skilled artisan will recognize that design of a peptide conjugated to any desired element comprises linkers that are all or partially flexible, such that the linker comprises a flexible linker as well as one or more portions that confer less flexible structure. Suitable linkers include proteins of 4 linked amino acids to 40 linked amino acids in length, or between 4 linked amino acids and 25 linked amino acids in length. In some embodiments, linked amino acids described herein comprise at least two amino acids linked by an amide bond.

In some embodiments, linkers are produced by using synthetic, linker-encoding oligonucleotides to couple proteins, or are encoded by a nucleic acid sequence encoding a fusion protein (e.g., an effector protein coupled to a fusion partner). In some embodiments, the linker is from 1 to 300, from 1 to 250, from 1 to 200, from 1 to 150, from 1 to 100, from 1 to 50, from 1 to 25, from 1 to 10, from 10 to 300, from 10 to 250, from 10 to 200, from 10 to 150, from 10 to 100, from 10 to 50, from 10 to 25, from 25 to 300, from 25 to 250, from 25 to 200, from 25 to 150, from 25 to 100, from 25 to 50, from 50 to 300, from 50 to 250, from 50 to 200, from 50 to 150, from 50 to 100, from 100 to 300, from 100 to 250, from 100 to 200, from 100 to 150, from 150 to 300, from 150 to 250, from 150 to 200, from 200 to 300, from 200 to 250, or from 250 to 300 amino acids in length. In some embodiments, the linker is from 1 to 100 amino acids in length. In some embodiments, the linker is more 100 amino acids in length. In some embodiments, the linker is from 10 to 27 amino acids in length. In some embodiments, linker proteins include glycine polymers (G)ₙ, glycine-serine polymers (including, for example, (GS)ₙ, GSGGSₙ (SEQ ID NO: 104), GGSGGSₙ (SEQ ID NO: 105), and GGGSₙ (SEQ ID NO: 106), where n is an integer of at least one), glycine-alanine polymers, and alanine-serine polymers. In some embodiments, exemplary linkers comprise amino acid sequences including, but not limited to, GGSG (SEQ ID NO: 107), GGSGG (SEQ ID NO: 108), GSGSG (SEQ ID NO: 109), GSGGG (SEQ ID NO: 110), GGGSG (SEQ ID NO: 111), and GSSSG (SEQ ID NO: 112). In some embodiments, the linker comprises one or more repeats a tri-peptide GGS. In some embodiments, the linker is a GS-rich linker. In some embodiments, the GS-rich linker comprises a peptide having two amino acids (2aa), three amino acids (3aa), five amino acids (5aa), ten amino acids (10aa), twenty amino acids (20aa), or forty amino acids (40aa). In some embodiments, the linker is an XTEN linker. In some embodiments, the XTEN linker is an XTEN80 linker. In some embodiments, the XTEN linker is an XTEN40 linker. In some embodiments, the XTEN linker is an XTEN20 linker. In some embodiments, the XTEN linker is an XTEN10 linker.

In some embodiments, a polypeptide described herein comprises an activity (e.g., a binding activity, a catalytic activity, or a combination thereof) for a target nucleic acid comprising a target strand and a non-target strand. In some embodiments, a length of the linker effects preference of the polypeptide for the activity on the target strand relative to the activity on the non-target strand. In some embodiments, a length of the linker effects preference of the polypeptide for the activity on the target strand relative to the activity on the non-target strand, wherein the polypeptide comprises C-terminus of an effector protein described herein linked by the linker to an effector protein described herein. In some embodiments, a shorter length of the linker (e.g., up to one amino acid, up to two amino acids, up to three amino acids, up to four amino acids, up to five amino acids, up to six amino acids, up to seven amino acids, up to eight amino acids, up to nine amino acids, or up to ten amino acids) favors activity of the polypeptide on the target strand relative to activity on the non-target strand, wherein the polypeptide comprises C-terminus of an effector protein described herein linked by the linker to an effector protein described herein.

In some embodiments, a length of a linker effects activity of the polypeptide described herein. In some embodiments, a length of the linker effects activity of the polypeptide, wherein the polypeptide comprises N-terminus of an effector protein described herein linked by the linker to an effector protein described herein.

In some embodiments, linkers do not comprise an amino acid. In some embodiments, linkers do not comprise a peptide. In some embodiments, linkers comprise a nucleotide, a polynucleotide, a polymer, or a lipid. In some embodiments, a linker comprises a polyethylene glycol (PEG), polypropylene glycol (PPG), co-poly(ethylene/propylene) glycol, polyoxyethylene (POE), polyurethane, polyphosphazene, polysaccharides, dextran, polyvinyl alcohol, polyvinylpyrrolidones, polyvinyl ethyl ether, polyacrylamide, polyacrylate, polycyanoacrylates, lipid polymers, chitins, hyaluronic acid, heparin, or an alkyl linker.

In some embodiments, a linker is recognized and cleaved by a protein. In some embodiments, a linker comprises a recognition sequence. In some embodiments, the recognition sequence is recognized and cleaved by the protein. In some embodiments, a guide nucleic acid comprises an aptamer. In some embodiments, the aptamer serves a similar function as a linker, bringing an effector protein and a fusion partner into proximity. In some embodiments, the aptamer functionally connects two proteins (e.g., effector protein, effector partners, fusion partner, fusion protein, or combinations thereof) by interacting non-covalently with both, thereby bringing both proteins into proximity of the guide nucleic acid. In some embodiments, the first protein and/or the second protein comprise or is covalently linked to an aptamer binding moiety. In some embodiments, the aptamer is a short single stranded RNA (ssRNA) molecule that binds the aptamer binding moiety. In some embodiments, the aptamer is a molecule that mimics antibody binding activity. In some embodiments, the aptamer is classified as a chemical antibody. In some instances, the aptamer described herein refers to artificial oligonucleotides that bind one or more specific molecules. In some embodiments, aptamers exhibit a range of affinities (K_{D} in the pM to μM range) with little or no off-target binding.

### Synthesis, Isolation and Assaying

Polypeptides (e.g., effector proteins, effector partners, fusion partners, fusion proteins, or combinations thereof) of the present disclosure are synthesized, using any suitable method. In some embodiments, the polypeptides are produced in vitro or by eukaryotic cells or by prokaryotic cells. In some embodiments, the polypeptides are further processed by unfolding (e.g., heat denaturation, dithiothreitol reduction, etc.) and may be further refolded, using any suitable method. In some embodiments, the nucleic acid(s) encoding the polypeptides described herein, the recombinant nucleic acid(s) described herein, the vectors described herein are produced in vitro or in vivo by eukaryotic cells or by prokaryotic cells.

Any suitable method of generating and assaying the polypeptides (e.g., effector proteins, effector partners, fusion partners, fusion proteins, or combinations thereof) described herein are used. Such methods include, but are not limited to, site-directed mutagenesis, random mutagenesis, combinatorial libraries, and other mutagenesis methods described herein (see, e.g., Sambrook et al., Molecular Cloning: A Laboratory Manual, Fourth Ed., Cold Spring Harbor Laboratory, Cold Spring Harbor (2012); Ausubel et al., Current Protocols in Molecular Biology, John Wiley and Sons, Baltimore, MD (1999); Gillman et al., Directed Evolution Library Creation: Methods and Protocols (Methods in Molecular Biology) Springer, 2^{nd} ed (2014)). One non-limiting example of a method for preparing the polypeptide is to express recombinant nucleic acids encoding the polypeptide in a suitable microbial organism, such as a bacterial cell, a yeast cell, or other suitable cell, using methods well known in the art. Exemplary methods are also described in the Examples provided herein.

In some embodiments, a polypeptide provided herein is an isolated polypeptide (e.g., effector protein, effector partner, fusion partner, fusion protein, or combinations thereof). In some embodiments, the polypeptide is isolated and purified for use in compositions, systems, devices, kits, and/or methods described herein. In some embodiments, methods described here comprise the step of isolating polypeptides described herein. Any suitable method to provide isolated polypeptides described herein is used in the present disclosure, for example, recombinant expression systems, precipitation, gel filtration, ion-exchange, reverse-phase and affinity chromatography, and the like. Other well-known methods are described in Deutscher et al., Guide to Protein Purification: Methods in Enzymology, Vol. 182, (Academic Press, (1990)). Alternatively, the isolated polypeptides of the present disclosure can be obtained using well-known recombinant methods (see, e.g., Sambrook et al., Molecular Cloning: A Laboratory Manual, Third Ed., Cold Spring Harbor Laboratory, New York (2001); and Ausubel et al., Current Protocols in Molecular Biology, John Wiley and Sons, Baltimore, MD (1999)). The methods and conditions for biochemical purification of a polypeptide described herein can be chosen by those skilled in the art, and purification monitored, for example, by a functional assay.

In some embodiments, compositions, systems, devices, kits, and methods described herein may further comprise a purification tag that can be attached to a polypeptide (e.g., effector protein, effector partner, fusion partner, fusion protein, or combinations thereof), or a nucleic acid encoding the purification tag that can be attached to a nucleic acid encoding the polypeptide as described herein. In some embodiments, the purification tag comprises an amino acid sequence which can attach or bind with high affinity to a separation substrate and assist in isolating the polypeptide of interest from its environment, which comprises its biological source, such as a cell lysate. Attachment of the purification tag is at the N or C terminus of the polypeptide. Furthermore, an amino acid sequence recognized by a protease or a nucleic acid encoding for an amino acid sequence recognized by a protease, such as TEV protease or the HRV3C protease is inserted between the purification tag and the polypeptide, such that biochemical cleavage of the amino acid sequence with the protease after initial purification liberates the purification tag. In some embodiments, purification and/or isolation are performed through high performance liquid chromatography (HPLC), exclusion chromatography, gel electrophoresis, affinity chromatography, or other purification technique. Non-limiting examples of purification tags are as described herein.

In some embodiments, polypeptides (e.g., effector proteins, effector partners, fusion partners, fusion proteins, or combinations thereof) described herein are isolated from cell lysate. In some embodiments, the compositions described herein comprise 20% or more by weight, 75% or more by weight, 95% or more by weight, 98% or more by weight, or 99.5% or more by weight of the polypeptide, related to the method of preparation of compositions described herein and its purification thereof, wherein percentages refer to total polypeptide content relative to contaminants. Thus, in some embodiments, the polypeptide is at least 80% pure, at least 85% pure, at least 90% pure, at least 95% pure, at least 98% pure, or at least 99% pure (e.g., free of contaminants, non-engineered proteins or other macromolecules, etc.) relative to the polypeptide.

### V. Nucleic Acid Systems

### Guide Nucleic Acids

The compositions, systems, devices, kits, and methods of the present disclosure may comprise a guide nucleic acid, a nucleic acid encoding the guide nucleic acid, or a use thereof. Unless otherwise indicated, compositions, systems, devices, kits, and methods comprising guide nucleic acids or uses thereof, as described herein and throughout, include DNA molecules, such as expression vectors, that encode a guide nucleic acid. Accordingly, in some embodiments, compositions, systems, and methods of the present disclosure comprise a guide nucleic acid or a nucleotide sequence encoding the guide nucleic acid. Guide nucleic acids are also referred to herein as “guide RNA.” A guide nucleic acid, as well as any components thereof (e.g., spacer sequence, repeat sequence, linker nucleotide sequence, etc.) comprise one or more deoxyribonucleotides, ribonucleotides, biochemically or chemically modified nucleotides (e.g., one or more engineered modifications as described herein), or any combinations thereof. Such nucleotide sequences described herein may be described as a nucleotide sequence of either DNA or RNA, however, no matter the form the sequence is described, it is readily understood that such nucleotide sequences can be revised to be RNA or DNA, as needed, for describing a sequence within a guide nucleic acid itself or the sequence that encodes a guide nucleic acid, such as a nucleotide sequence described herein for a vector. Similarly, disclosure of the nucleotide sequences described herein also discloses the complementary nucleotide sequence, the reverse nucleotide sequence, and the reverse complement nucleotide sequence, any one of which can be a nucleotide sequence for use in a guide nucleic acid as described herein. In some embodiments, a guide nucleic acid sequence(s) comprises one or more nucleotide alterations at one or more positions in any one of the sequences described herein. Alternative nucleotides can be any one or more of A, C, G, T or U, or a deletion, or an insertion. As described in further detailed herein, any one or more nucleotide in a guide nucleic acid sequence can be modified (see e.g., Engineered Modification section en infra). For example, a guide nucleic acid sequence can comprise one or more pseudouridine modifications. Accordingly, a person of ordinary skill in the art would understand that any description of “U” in a nucleotide sequence of a guide nucleic acid can refer to uracil or 1N-Methyl-Pseudouridine.

In some embodiments, a guide nucleic acid comprises a naturally occurring sequence. In some embodiments, a guide nucleic acid comprises a non-naturally occurring sequence, wherein the nucleotide sequence of the guide nucleic acid, or any portion thereof, is different from the nucleotide sequence of a naturally occurring guide nucleic acid. A guide nucleic acid of the present disclosure comprises one or more of the following: a) a single nucleic acid molecule; b) a DNA base; c) an RNA base; d) a modified base; e) a modified sugar; and f) a modified backbone. Modifications are described herein and throughout the present disclosure (e.g., in the section entitled “Engineered Modifications”). In some embodiments, a guide nucleic acid is chemically synthesized or recombinantly produced by any suitable methods. In some embodiments, guide nucleic acids and portions thereof are found in or identified from a CRISPR array present in the genome of a host organism or cell.

In general, the guide nucleic acid comprises a nucleotide sequence that is at least 80%, at least 85%, at least 90%, at least 95%, at least 98%, at least 99%, or at least 100% complementary to the target sequence. In general, a portion of the guide nucleic acid (i.e., the spacer sequence) has a degree of complementarity to the target sequence and hybridizes to the target sequence. In some embodiments, the guide nucleic acid comprises at least 10 contiguous nucleotides that are complementary to the target sequence in the target nucleic acid. In some embodiments, guide nucleic acid comprises a spacer sequence that is at least 80%, at least 85%, at least 90%, at least 95%, at least 98%, at least 99%, or at least 100% complementary to the target sequence.

In general, a guide nucleic acid comprises a first region or sequence that is not complementary to a target nucleic acid (FR) and a second region or sequence is complementary to the target nucleic acid (SR), wherein the FR and the SR are heterologous to each other. In some embodiments, FR is located 5′ to SR (FR-SR). In some embodiments, SR is located 5′ to FR (SR-FR). In some embodiments, at least a portion of the FR interacts or binds to an effector protein. In some embodiments, the SR comprises a spacer sequence, wherein the spacer sequence can interact in a sequence-specific manner with (e.g., has complementarity with, or can hybridize to a target sequence in) a target nucleic acid. In some embodiments, the first region or sequence interacts with the effector protein (e.g., polypeptide). In some embodiments, the first region or sequence is covalently linked to the 5′ end of the second region or sequence.

In some embodiments, the first region or sequence, the second region or sequence, or both are about 8 nucleic acids, about 10 nucleic acids, about 12 nucleic acids, about 14 nucleic acids, about 16 nucleic acids, about 18 nucleic acids, about 20 nucleic acids, about 22 nucleic acids, about 24 nucleic acids, about 26 nucleic acids, about 28 nucleic acids, about 30 nucleic acids, about 32 nucleic acids, about 34 nucleic acids, about 36 nucleic acids, about 38 nucleic acids, about 40 nucleic acids, about 42 nucleic acids, about 44 nucleic acids, about 46 nucleic acids, about 48 nucleic acids, or about 50 nucleic acids long.

In some embodiments, the first region or sequence, the second region or sequence, or both are from about 8 to about 12, from about 8 to about 16, from about 8 to about 20, from about 8 to about 24, from about 8 to about 28, from about 8 to about 30, from about 8 to about 32, from about 8 to about 34, from about 8 to about 36, from about 8 to about 38, from about 8 to about 40, from about 8 to about 42, from about 8 to about 44, from about 8 to about 48, or from about 8 to about 50 nucleic acids long.

In some embodiments, the first region or sequence, the second region or sequence, or both comprise a GC content of about 1%, about 5%, about 10%, about 15%, about 20%, about 25%, about 30%, about 40%, about 50%, about 60%, about 70%, about 80%, about 90%, about 95%, about 98%, or about 99%. In some embodiments, the first region or sequence, the second region or sequence, or both may comprise a GC content of from about 1% to about 95%, from about 5% to about 90%, from about 10% to about 80%, from about 15% to about 70%, from about 20% to about 60%, from about 25% to about 50%, or from about 30% to about 40%.

In some embodiments, the first region or sequence, the second region or sequence, or both have a melting temperature of about 38° C., about 40° C., about 42° C., about 44° C., about 46° C., about 48° C., about 50° C., about 52° C., about 54° C., about 56° C., about 58° C., about 60° C., about 62° C., about 64° C., about 66° C., about 68° C., about 70° C., about 72° C., about 74° C., about 76° C., about 78° C., about 80° C., about 82° C., about 84° C., about 86° C., about 88° C., about 90° C., or about 92° C. In some embodiments, the first region or sequence, the second region or sequence, or both may have a melting temperature of from about 35° C. to about 40° C., from about 35° C. to about 45° C., from about 35° C. to about 50° C., from about 35° C. to about 55° C., from about 35° C. to about 60° C., from about 35° C. to about 65° C., from about 35° C. to about 70° C., from about 35° C. to about 75° C., from about 35° C. to about 80° C., or from about 35° C. to about 85° C.

In some embodiments, the guide nucleic acid also forms complexes as described through herein. For example, in some embodiments, a guide nucleic acid hybridizes to another nucleic acid, such as target nucleic acid, or a portion thereof. In some embodiments, a portion of the guide nucleic acid hybridizes to a portion of a target nucleic acid. In another example, a guide nucleic acid complexes with an effector protein. In such embodiments, a guide nucleic acid-effector protein complex is described herein as an RNP. In some embodiments, when in a complex, at least a portion of the complex binds, recognizes, and/or hybridizes to a target nucleic acid. For example, when a guide nucleic acid and an effector protein are complexed to form an RNP, at least a portion of the guide nucleic acid hybridizes to a target sequence in a target nucleic acid. Those skilled in the art in reading the below specific examples of guide nucleic acids as used in RNPs described herein, will understand that in some embodiments, a RNP hybridizes to one or more target sequences in a target nucleic acid, thereby allowing the RNP to modify and/or recognize a target nucleic acid or sequence contained therein or to modify and/or recognize non-target sequences depending on the guide nucleic acid, and in some embodiments, the effector protein, used.

In some embodiments, a guide nucleic acid comprises or forms intramolecular secondary structure (e.g., hairpins, stem-loops, etc.). In some embodiments, a guide nucleic acid comprises a stem-loop structure comprising a stem region and a loop region. In some embodiments, the stem region is 4 to 8 linked nucleotides in length. In some embodiments, the stem region is 5 to 6 linked nucleotides in length. In some embodiments, the stem region is 4 to 5 linked nucleotides in length. In some embodiments, the guide nucleic acid comprises a pseudoknot (e.g., a secondary structure comprising a stem, at least partially, hybridized to a second stem or half-stem secondary structure). In some embodiments, an effector protein recognizes a guide nucleic acid comprising multiple stem regions. In some embodiments, the nucleotide sequences of the multiple stem regions are identical to one another. In some embodiments, the nucleotide sequences of at least one of the multiple stem regions is not identical to those of the others. In some embodiments, the guide nucleic acid comprises at least 2, at least 3, at least 4, or at least 5 stem regions.

In some embodiments, the compositions, systems, devices, kits, and methods of the present disclosure comprise two or more guide nucleic acids (e.g., 2, 3, 4, 5, 6, 7, 9, 10 or more guide nucleic acids), and/or uses thereof. In some embodiments, multiple guide nucleic acids target an effector protein to different locations in the target nucleic acid by hybridizing to different target sequences. In some embodiments, a first guide nucleic acid hybridizes within a location of the target nucleic acid that is different from where a second guide nucleic acid hybridizes the target nucleic acid. In some embodiments, the first loci and the second loci of the target nucleic acid are located at least 1, at least 10, at least 20, at least 30, at least 40, at least 50, at least 60, at least 70, at least 80, at least 90 or at least 100 nucleotides apart. In some embodiments, the first loci and the second loci of the target nucleic acid are located between 100 and 200, 200 and 300, 300 and 400, 400 and 500, 500 and 600, 600 and 700, 700 and 800, 800 and 900 or 900 and 1,000 nucleotides apart.

In some embodiments, the first loci and/or the second loci of the target nucleic acid are located in an intron of a gene. In some embodiments, the first loci and/or the second loci of the target nucleic acid are located in an exon of a gene. In some embodiments, the first loci and/or the second loci of the target nucleic acid span an exon-intron junction of a gene. In some embodiments, the first portion and/or the second portion of the target nucleic acid are located on either side of an exon and cutting at both sites results in deletion of the exon. In some embodiments, compositions, systems, and methods comprising multiple guide nucleic acids or uses thereof comprise multiple effector proteins, wherein the effector proteins are identical, non-identical, or combinations thereof.

In some embodiments, a guide nucleic acid comprises about: 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 linked nucleotides. In general, a guide nucleic acid comprises at least: 10, 15, 20, 25, 30, 35, 40, 45, 50, 55, 60 linked nucleotides. In some embodiments, the guide nucleic acid has about 10 to about 60, about 20 to about 50, or about 30 to about 40 linked nucleotides.

In some embodiments, a guide nucleic acid comprises at least 10, 11, 12, 13, 14, 15, 16, 17, 18, 19, 20, 21, 22, 23, 24, 25, 26, 27, 28, 29, or 30 contiguous nucleotides that are complementary to a eukaryotic sequence. Such a eukaryotic sequence is a nucleotide sequence that is present in a host eukaryotic cell. Such a nucleotide sequence is distinguished from nucleotide sequences present in other host cells, such as prokaryotic cells, or viruses. Said sequences present in a eukaryotic cell can be located in a gene, an exon, an intron, a non-coding (e.g., promoter or enhancer) region, a selectable marker, tag, or signal. In some embodiments, a target sequence is a eukaryotic sequence.

In some embodiments, a length of a guide nucleic acid is about 30 to about 120 linked nucleotides. In some embodiments, the length of a guide nucleic acid is about 40 to about 100, about 40 to about 90, about 40 to about 80, about 40 to about 70, about 40 to about 60, about 40 to about 50, about 50 to about 90, about 50 to about 80, about 50 to about 70, or about 50 to about 60 linked nucleotides. In some embodiments, the length of a guide nucleic acid is about 40, about 45, about 50, about 55, about 60, about 65, about 70 or about 75 linked nucleotides. In some embodiments, the length of a guide nucleic acid is greater than about 15, about 20, about 25, about 30, about 35, about 40, about 45, about 50, about 55, about 60, about 65, about 70 or about 75 linked nucleotides. In some embodiments, the length of a guide nucleic acid is not greater than about 40, about 45, about 50, about 55, about 60, about 65, about 70, about 75, about 80, about 85, about 90, about 95, about 100, about 105, about 110, about 115, about 120, or about 125 linked nucleotides.

In some embodiments, guide nucleic acids comprise additional elements that contribute additional functionality (e.g., stability, heat resistance, etc.) to the guide nucleic acid. In some embodiments, the elements comprise one or more nucleotide alterations, nucleotide sequences, intermolecular secondary structures, or intramolecular secondary structures (e.g., one or more hair pin regions, one or more bulges, etc.).

In some embodiments, guide nucleic acids comprise one or more linkers connecting different nucleotide sequences as described herein. In some embodiments, a linker comprises 1, 2, 3, 4, 5, 6, 7, 8, 9, 10, or more nucleotides. In some embodiments, a linker comprises any suitable linker, examples of which are described herein.

In some embodiments, guide nucleic acids comprise one or more nucleotide sequences as described herein (e.g., TABLE 3). In some embodiments, the nucleotide sequences described herein (e.g., TABLE 3) are described as a nucleotide sequence of either DNA or RNA, however, no matter the form of the nucleotide sequence described, it is readily understood that such nucleotide sequences may be revised to be RNA or DNA, as needed, for describing a sequence within a guide nucleic acid itself or the nucleotide sequence that encodes a guide nucleic acid, such as a nucleotide sequence described herein for a vector. Similarly, disclosure of the nucleotide sequences described herein (e.g., TABLE 3) also describes the complementary nucleotide sequence, the reverse nucleotide sequence, and the reverse complement nucleotide sequence, any one of which may be a nucleotide sequence for use in a guide nucleic acid as described herein. In some embodiments, guide nucleic acid sequence(s) comprises one or more nucleotide alterations at one or more positions in any one of the nucleotide sequences described herein. Alternative nucleotides may be any one or more of A, C, G, T or U, or a deletion, or an insertion. As described in further detailed herein, any one or more nucleotide in a sequence comprised in a guide nucleic acid sequence can be modified (see e.g., Engineered Modification section en infra). For example, a sequence comprised in a guide nucleic acid can comprise one or more pseudouridine modifications. Accordingly, a person of ordinary skill in the art would understand that any description of “U” in a nucleotide sequence comprised in a guide nucleic acid can refer to uracil or 1N-Methyl-Pseudouridine.

In some embodiments, the guide nucleic acid comprises a nucleotide sequence that hybridizes to a target sequence in a target nucleic acid, wherein the target nucleic acid is an RNA. In some embodiments, the target nucleic acid is a double-stranded RNA or a single-stranded RNA. In some embodiments, the target nucleic acid is linear single-stranded RNA, or circular RNA.

In some embodiments, the guide nucleic acid comprises a nucleotide sequence that is capable of hybridizing to a target sequence in a target nucleic acid, wherein the target nucleic acid comprises any one of: a naturally occurring eukaryotic sequence, a naturally occurring prokaryotic sequence, a naturally occurring viral sequence, a naturally occurring bacterial sequence, a naturally occurring fungal sequence, an engineered eukaryotic sequence, an engineered prokaryotic sequence, an engineered viral sequence, an engineered bacterial sequence, an engineered fungal sequence, a fragment of a naturally occurring sequence, a fragment of an engineered sequence, and combinations thereof.

In some embodiments, the target nucleic acid is isolated from any one of: a naturally occurring cell, a eukaryotic cell, a prokaryotic cell, a plant cell, a fungal cell, an animal cell, cell of an invertebrate, a fly cell, a cell of a vertebrate, a mammalian cell, a primate cell, a non-human primate cell, a human cell, a living cell, a non-living cell, a modified cell, a derived cell, and a non-naturally occurring cell.

### A Single Nucleic Acid System

In some embodiments, compositions, systems, devices, kits, and methods described herein comprise a single nucleic acid system comprising a guide nucleic acid or a nucleotide sequence encoding the guide nucleic acid, and one or more effector proteins or a nucleotide sequence encoding the one or more effector proteins. In some embodiments, a first region (FR) or sequence of the guide nucleic acid non-covalently interacts with the one or more polypeptides described herein. In some embodiments, a second region (SR) or sequence of the guide nucleic acid hybridizes with a target sequence of the target nucleic acid. In the single nucleic acid system having a complex of the guide nucleic acid and the effector protein, the effector protein is not transactivated by the guide nucleic acid. In other words, activity of effector protein does not require binding to a second non-target nucleic acid molecule. An exemplary guide nucleic acid for a single nucleic acid system is a crRNA.

crRNA

In some embodiments, a guide nucleic acid comprises a crRNA. In some embodiments, the guide nucleic acid is the crRNA. In general, a crRNA comprises a first region (FR) or sequence and a second region (SR) or sequence, wherein the FR of the crRNA comprises a repeat sequence, and the SR of the crRNA comprises a spacer sequence. In some embodiments, the repeat sequence and the spacer sequences are directly connected to each other (e.g., covalent bond (phosphodiester bond)). In some embodiments, the repeat sequence and the spacer sequence are connected by a linker.

In some embodiments, a crRNA comprises deoxyribonucleosides, ribonucleosides, chemically modified nucleosides, or any combination thereof. In some embodiments, a crRNA comprises about: 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 linked nucleotides. In some embodiments, a crRNA comprises at least: 10, 15, 20, 25, 30, 35, 40, 45, 50, 55, 60 linked nucleotides. In some embodiments, the length of the crRNA is about 20 to about 120 linked nucleotides. In some embodiments, the length of a crRNA is about 20 to about 100, about 30 to about 100, about 40 to about 100, about 40 to about 90, about 40 to about 80, about 40 to about 70, about 40 to about 60, about 40 to about 50, about 50 to about 90, about 50 to about 80, about 50 to about 70, or about 50 to about 60 linked nucleotides. In some embodiments, the length of a crRNA is about 20, about 25, about 30, about 35, about 40, about 45, about 50, about 55, about 60, about 65, about 70 or about 75 linked nucleotides.

### Repeat Sequence

In some embodiments, guide nucleic acids described herein comprise one or more repeat sequences. In some embodiments, a repeat sequence comprises a nucleotide sequence that is not complementary to a target sequence of a target nucleic acid. In some embodiments, a repeat sequence comprises a nucleotide sequence that interacts with an effector protein. In some embodiments, a repeat sequence is connected to another sequence of a guide nucleic acid that non-covalently interacts with an effector protein. In some embodiments, a repeat sequence includes a nucleotide sequence that forms a guide nucleic acid-effector protein complex (e.g., a RNP complex).

In some embodiments, the repeat sequence is between 10 and 50, 12 and 48, 14 and 46, 16 and 44, and 18 and 42 nucleotides in length.

In some embodiments, a repeat sequence is adjacent to a spacer sequence. In some embodiments, a repeat sequence is followed by a spacer sequence in the 5′ to 3′ direction. In some embodiments, a repeat sequence is preceded by a spacer sequence in the 5′ to 3′ direction. In some embodiments, a repeat sequence is followed by a spacer sequence in the 5′ to 3′ direction. In some embodiments, a repeat sequence is linked to a spacer sequence. In some embodiments, a guide nucleic acid comprises a repeat sequence linked to a spacer sequence by a direct link or by any suitable linker, examples of which are described herein.

In some embodiments, guide nucleic acids comprise more than one repeat sequence (e.g., two or more, three or more, or four or more repeat sequences). In some embodiments, a guide nucleic acid comprises more than one repeat sequence separated by another sequence of the guide nucleic acid. For example, in some embodiments, a guide nucleic acid comprises two repeat sequences, wherein the first repeat sequence is followed by a spacer sequence, and the spacer sequence is followed by a second repeat sequence in the 5′ to 3′ direction. In some embodiments, the more than one repeat sequences are identical. In some embodiments, the more than one repeat sequences are not identical.

In some embodiments, the repeat sequence comprises two sequences that are complementary to each other and hybridize to form a double stranded RNA duplex (dsRNA duplex). In some embodiments, the two sequences are not directly linked and hybridize to form a stem loop structure. In some embodiments, the dsRNA duplex comprises 5, 10, 15, 20 or 25 base pairs (bp). In some embodiments, not all nucleotides of the dsRNA duplex are paired, and therefore the duplex forming sequence comprises a bulge. In some embodiments, the repeat sequence comprises a hairpin or stem-loop structure, optionally at the 5′ portion of the repeat sequence. In some embodiments, a strand of the stem portion comprises a sequence and the other strand of the stem portion comprises a sequence that is, at least partially, complementary. In some embodiments, such sequences comprise 65% to 100% complementarity (e.g., 65%, 70%, 75%, 80%, 85%, 90%, 95%, 98%, or 100% complementarity). In some embodiments, a guide nucleic acid comprises a nucleotide sequence that, when involved in hybridization events, hybridizes over one or more segments of a target nucleic acid such that intervening or adjacent segments are not involved in the hybridization event (e.g., a bulge, a loop structure or hairpin structure, etc.).

In some embodiments, a repeat sequence comprises a nucleotide sequence that is at least 65%, at least 70%, at least 75%, at least 80%, at least 85%, at least 90%, at least 95%, at least 97%, or at least 98%, at least 99%, or at least 100% identical to an equal length portion of any one of the repeat sequences in TABLE 3. In some embodiments, a repeat sequence comprises at least 5, at least 6, at least 7, at least 8, at least 9, at least 10, at least 11, at least 12, at least 13, at least 14, at least 15, at least 16, at least 17, at least 18, at least 19, at least 20, or at least 21 contiguous nucleotides of any one of the sequences recited in TABLE 3.

In some embodiments, a repeat sequence comprises one or more nucleotide alterations at one or more positions in the sequence recited in TABLE 3. Alternative nucleotides can be any one or more of A, C, G, T or U, or a deletion, or an insertion. As described in further detailed herein, any one or more nucleotide in a repeat sequence can be modified (see e.g., Engineered Modification section en infra). For example, a repeat sequence can comprise one or more pseudouridine modifications. Accordingly, a person of ordinary skill in the art would understand that any description of “U” in a nucleotide sequence comprised in a repeat sequence can refer to uracil or 1N-Methyl-Pseudouridine.

In some embodiments, the repeat sequence is at least 75% identical to any one of sequences set forth in TABLE 3. In some embodiments, the engineered guide nucleic acid comprises a repeat sequence and wherein; (a) the polypeptide comprises an amino acid sequence that is at least 85% identical to SEQ ID NO: 1 and wherein the repeat sequence comprises a nucleotide sequence that is at least 75% identical to SEQ ID NO: 30 or SEQ ID NO: 50; (b) the polypeptide comprises an amino acid sequence that is at least 85% identical to SEQ ID NO: 2 and wherein the repeat sequence comprises a nucleotide sequence that is at least 75% identical to SEQ ID NO: 31, SEQ ID NO: 35, or SEQ ID NO: 47; (c) the polypeptide comprises an amino acid sequence that is at least 85% identical to SEQ ID NO: 3 and wherein the repeat sequence comprises a nucleotide sequence that is at least 75% identical to SEQ ID NO: 32 or SEQ ID NO: 39; (d) the polypeptide comprises an amino acid sequence that is at least 85% identical to SEQ ID NO: 4 and wherein the repeat sequence comprises a nucleotide sequence that is at least 75% identical to SEQ ID NO: 33 or SEQ ID NO: 44; (e) the polypeptide comprises an amino acid sequence that is at least 85% identical to SEQ ID NO: 5 and wherein the repeat sequence comprises a nucleotide sequence that is at least 75% identical to SEQ ID NO: 34, SEQ ID NO: 40, or SEQ ID NO: 48; (f) the polypeptide comprises an amino acid sequence that is at least 85% identical to SEQ ID NO: 6 and wherein the repeat sequence comprises a nucleotide sequence that is at least 75% identical to SEQ ID NO: 35 or SEQ ID NO: 31; (g) the polypeptide comprises an amino acid sequence that is at least 85% identical to SEQ ID NO: 7 and wherein the repeat sequence comprises a nucleotide sequence that is at least 75% identical to SEQ ID NO: 36, SEQ ID NO: 48, or SEQ ID NO: 52; (h) the polypeptide comprises an amino acid sequence that is at least 85% identical to SEQ ID NO: 8 and wherein the repeat sequence comprises a nucleotide sequence that is at least 75% identical to SEQ ID NO: 37 or SEQ ID NO: 42; (i) the polypeptide comprises an amino acid sequence that is at least 85% identical to SEQ ID NO: 9 and wherein the repeat sequence comprises a nucleotide sequence that is at least 75% identical to SEQ ID NO: 38 or SEQ ID NO: 58; (j) the polypeptide comprises an amino acid sequence that is at least 85% identical to SEQ ID NO: 10 and wherein the repeat sequence comprises a nucleotide sequence that is at least 75% identical to SEQ ID NO: 39, SEQ ID NO: 32, or SEQ ID NO: 49; (k) the polypeptide comprises an amino acid sequence that is at least 85% identical to SEQ ID NO: 11 and wherein the repeat sequence comprises a nucleotide sequence that is at least 75% identical to SEQ ID NO: 40 or SEQ ID NO: 34; (l) the polypeptide comprises an amino acid sequence that is at least 85% identical to SEQ ID NO: 12 and wherein the repeat sequence comprises a nucleotide sequence that is at least 75% identical to SEQ ID NO: 41 or SEQ ID NO: 46; (m) the polypeptide comprises an amino acid sequence that is at least 85% identical to SEQ ID NO: 13 and wherein the repeat sequence comprises a nucleotide sequence that is at least 75% identical to SEQ ID NO: 42; (n) the polypeptide comprises an amino acid sequence that is at least 85% identical to SEQ ID NO: 14 and wherein the repeat sequence comprises a nucleotide sequence that is at least 75% identical to SEQ ID NO: 43 or SEQ ID NO: 30; (o) the polypeptide comprises an amino acid sequence that is at least 85% identical to SEQ ID NO: 15 and wherein the repeat sequence comprises a nucleotide sequence that is at least 75% identical to SEQ ID NO: 44 or SEQ ID NO: 33; (p) the polypeptide comprises an amino acid sequence that is at least 85% identical to SEQ ID NO: 68 and wherein the repeat sequence comprises a nucleotide sequence that is at least 75% identical to SEQ ID NO: 48 or SEQ ID NO: 59; (q) the polypeptide comprises an amino acid sequence that is at least 85% identical to SEQ ID NO: 69 and wherein the repeat sequence comprises a nucleotide sequence that is at least 75% identical to SEQ ID NO: 57 or SEQ ID NO: 60; (r) the polypeptide comprises an amino acid sequence that is at least 85% identical to SEQ ID NO: 70 and wherein the repeat sequence comprises a nucleotide sequence that is at least 75% identical to SEQ ID NO: 61; (s) the polypeptide comprises an amino acid sequence that is at least 85% identical to SEQ ID NO: 71 and wherein the repeat sequence comprises a nucleotide sequence that is at least 75% identical to SEQ ID NO: 62; (t) the polypeptide comprises an amino acid sequence that is at least 85% identical to SEQ ID NO: 72 and wherein the repeat sequence comprises a nucleotide sequence that is at least 75% identical to SEQ ID NO: 63; (u) the polypeptide comprises an amino acid sequence that is at least 85% identical to SEQ ID NO: 73 and wherein the repeat sequence comprises a nucleotide sequence that is at least 75% identical to SEQ ID NO: 64; (v) the polypeptide comprises an amino acid sequence that is at least 85% identical to SEQ ID NO: 74 and wherein the repeat sequence comprises a nucleotide sequence that is at least 75% identical to SEQ ID NO: 65; (w) the polypeptide comprises an amino acid sequence that is at least 85% identical to SEQ ID NO: 75 and wherein the repeat sequence comprises a nucleotide sequence that is at least 75% identical to SEQ ID NO: 66; (x) the polypeptide comprises an amino acid sequence that is at least 85% identical to SEQ ID NO: 76 and wherein the repeat sequence comprises a nucleotide sequence that is at least 75% identical to SEQ ID NO: 67; (y) the polypeptide comprises an amino acid sequence that is at least 85% identical to SEQ ID NO: 93 and wherein the repeat sequence comprises a nucleotide sequence that is at least 75% identical to SEQ ID NO: 45; (z) the polypeptide comprises an amino acid sequence that is at least 85% identical to any one of SEQ ID NO: 94-97 and wherein the repeat sequence comprises a nucleotide sequence that is at least 75% identical to SEQ ID NO: 53; (aa) the polypeptide comprises an amino acid sequence that is at least 85% identical to SEQ ID NO: 97 and wherein the repeat sequence comprises a nucleotide sequence that is at least 75% identical to SEQ ID NO: 54; (bb) the polypeptide comprises an amino acid sequence that is at least 85% identical to SEQ ID NO: 98 or 99 and wherein the repeat sequence comprises a nucleotide sequence that is at least 75% identical to SEQ ID NO: 55; or (cc) the polypeptide comprises an amino acid sequence that is at least 85% identical to any one of SEQ ID NO: 90, 91, 92, 98, or 99 and wherein the repeat sequence comprises a nucleotide sequence that is at least 75% identical to SEQ ID NO: 56.

### Spacer Sequence

In some embodiments, guide nucleic acids described herein comprise one or more spacer sequences. In some embodiments, a spacer sequence hybridizes to a target sequence of a target nucleic acid. In some embodiments, a spacer sequence comprises a nucleotide sequence that is, at least partially, hybridizable to an equal length of a sequence (e.g., a target sequence) of a target nucleic acid. Exemplary hybridization conditions are described herein. In some embodiments, the spacer sequence functions to direct an RNP complex comprising the guide nucleic acid to the target nucleic acid for detection and/or modification. In some embodiments, the spacer sequence functions to direct a RNP to the target nucleic acid for detection and/or modification. In some embodiments, a spacer sequence is complementary to a target sequence that is adjacent to a PAM that is recognizable by an effector protein described herein.

In some embodiments, a spacer sequence comprises at least 5 to about 50 contiguous nucleotides that are complementary to a target sequence in a target nucleic acid. In some embodiments, a spacer sequence comprises at least 5 to about 50 linked nucleotides. In some embodiments, a spacer sequence comprises at least 5 to about 50, at least 5 to about 25, at least about 10 to at least about 25, or at least about 15 to about 25 linked nucleotides. In some embodiments, the spacer sequence comprises 15-28 linked nucleotides. In some embodiments, a spacer sequence comprises 15-26, 15-24, 15-22, 15-20, 15-18, 16-28, 16-26, 16-24, 16-22, 16-20, 16-18, 17-26, 17-24, 17-22, 17-20, 17-18, 18-26, 18-24, or 18-22 linked nucleotides. In some embodiments, the spacer sequence comprises 10, 11, 12, 13, 14, 15, 16, 17, 18, 19, 20, 21, 22, 23, 24, 25, 26, 27, 28, 29, 30, or more nucleotides.

In some embodiments, a spacer sequence is adjacent to a repeat sequence. In some embodiments, a spacer sequence follows a repeat sequence in a 5′ to 3′ direction. In some embodiments, a spacer sequence precedes a repeat sequence in a 5′ to 3′ direction. In some embodiments, the spacer sequence(s) and the repeat sequence(s) of the guide nucleic acid are present within the same molecule. In some embodiments, the spacer(s) and repeat sequence(s) are linked directly to one another. In some embodiments, a linker is present between the spacer(s) and repeat sequences. In some embodiments, linkers may be any suitable linker. In some embodiments, the spacer sequence(s) and the repeat sequence(s) of the guide nucleic acid are present in separate molecules, which are joined to one another by base pairing interactions.

In some embodiments, a spacer sequence comprises a nucleotide sequence that is at least 65%, at least 70%, at least 75%, at least 80%, at least 85%, at least 90%, at least 95%, at least 98%, or at least 100% complementary to a target sequence of a target nucleic acid. A spacer sequence hybridizes to an equal length portion of a target nucleic acid (e.g., a target sequence). In some embodiments, a target nucleic acid, such as RNA, is associated with a cancer or a genetic disorder, or an amplicon thereof, as described herein. In some embodiments, a target nucleic acid is associated with a gene selected from TABLE 5 and TABLE 5.1. In some embodiments, a spacer sequence comprises a nucleotide sequence that is at least 65%, at least 70%, at least 75%, at least 80%, at least 85%, at least 90%, at least 95% or 100% complementary to a target sequence associated with a nucleic acid selected from TABLE 4 and TABLE 4.1. In some embodiments, a target nucleic acid is a nucleic acid associated with a disease or syndrome set forth in TABLE 5 and TABLE 5.1. In some embodiments, a spacer sequence comprises a nucleotide sequence that is at least 65%, at least 70%, at least 75%, at least 80%, at least 85%, at least 90%, at least 95%, at least 98%, or at least 100% complementary to a target sequence of a target nucleic acid associated with a disease or syndrome set forth in TABLE 5 and TABLE 5.1. In some embodiments, the spacer sequence comprises at least 10, 11, 12, 13, 14, 15, 16, 17, 18, 19, 20, 21, 22, 23, 24, 25, 26, 27, 28, 29, or 30 contiguous nucleotides that hybridizes to the target sequence. In some embodiments, the spacer sequence comprises at least 10, 11, 12, 13, 14, 15, 16, 17, 18, 19, 20, 21, 22, 23, 24, 25, 26, 27, 28, 29, or 30 contiguous nucleotides that are complementary to the target sequence.

It is understood that the nucleotide sequence of a spacer sequence need not be 100% complementary to that of a target sequence of a target nucleic acid to hybridize or hybridize specifically to the target sequence. For example, the spacer sequence, in some embodiments, comprises at least one alteration, such as a substituted or modified nucleotide, that is not complementary to the corresponding nucleotide of the target sequence. As described in further detail herein, any one or more nucleotide in a spacer sequence can be modified (see e.g., Engineered Modification section en infra). For example, a spacer sequence can comprise one or more pseudouridine modifications. Accordingly, a person of ordinary skill in the art would understand that any description of “U” in a nucleotide sequence comprised in a spacer sequence can refer to uracil or 1N-Methyl-Pseudouridine. Spacer sequences are further described throughout herein.

### Pooling Guide Nucleic Acids

In some embodiments, a plurality of guide nucleic acids are provided herein that are pooled for use in compositions, systems, devices, and/or methods described herein. Pooling guide nucleic acids includes adding multiple guide nucleic acids to a complex master mix in a complexing reaction or a detection reaction. In some embodiments, pooling involves multiple guide nucleic acids designed to target and/or hybridize to different target sequences or different sequence segments of the same target nucleic acid. Thus, pooling can broaden the detection spectrum in a single reaction and increase the detection efficiency. Accordingly, in some embodiments, compositions, systems, devices, and/or methods described herein comprise pooling a plurality of guide nucleic acids, wherein each of a plurality of guide nucleic acids (e.g., a single nucleic acid system comprising a guide nucleic acid (e.g., sgRNA or crRNA)) are complexed to an effector protein forming multiple different effector protein-guide nucleic acid complexes.

### VI. Engineered Modifications

Polypeptides (e.g., effector proteins) and nucleic acids (e.g., engineered guide nucleic acids) can be further modified as described herein. Examples are modifications that do not alter the primary sequence of the polypeptides or nucleic acids, such as chemical derivatization of polypeptides (e.g., acylation, acetylation, carboxylation, amidation, etc.), or modifications that do alter the primary sequence of the polypeptide or nucleic acid. Also included are polypeptides that have a modified glycosylation pattern (e.g., those made by: modifying the glycosylation patterns of a polypeptide during its synthesis and processing or in further processing steps; by exposing the polypeptide to enzymes which affect glycosylation, such as mammalian glycosylating or deglycosylating enzymes). Also embraced are polypeptides that have phosphorylated amino acid residues (e.g., phosphotyrosine, phosphoserine, or phosphothreonine).

Modifications disclosed herein can also include modification of described polypeptides and/or guide nucleic acids through any suitable method, such as molecular biological techniques and/or synthetic chemistry, to improve their resistance to proteolytic degradation, to change the target sequence specificity, to optimize solubility properties, to alter protein activity (e.g., transcription modulatory activity, enzymatic activity, etc.) or to render them more suitable for their intended purpose (e.g., in vivo administration, in vitro methods, or ex vivo applications). Analogs of such polypeptides include those containing residues other than naturally occurring L-amino acids, e.g., D-amino acids or non-naturally occurring synthetic amino acids. In some embodiments, D-amino acids is substituted for some or all of the amino acid residues. Modifications can also include modifications with non-naturally occurring unnatural amino acids. The particular sequence and the manner of preparation will be determined by convenience, economics, or purity required.

Modifications can further include the introduction of various groups to polypeptides and/or guide nucleic acids described herein. For example, groups can be introduced during synthesis or during expression of a polypeptide (e.g., an effector protein), which allow for linking to other molecules or to a surface. Thus, in some embodiments, cysteines are used to make thioethers, histidines for linking to a metal ion complex, carboxyl groups for forming amides or esters, or amino groups for forming amides.

Modifications can further include changing of nucleic acids described herein (e.g., engineered guide nucleic acids) to provide the nucleic acid with a new or enhanced feature, such as improved stability. Such modifications of a nucleic acid include a base editing, a base modification, a backbone modification, a sugar modification, or combinations thereof. In some embodiments, the modifications can be of one or more nucleotides, nucleosides, or nucleobases in a nucleic acid.

In some embodiments, nucleic acids (e.g., nucleic acids encoding effector proteins, engineered guide nucleic acids, or nucleic acids encoding engineered guide nucleic acids) described herein comprise one or more modifications comprising: 2′O-methyl modified nucleotides (e.g., 2′-O-Methyl (2′OMe) sugar modifications); 2′ fluoro modified nucleotides (e.g., 2′-fluoro (2′-F) sugar modifications); locked nucleic acid (LNA) modified nucleotides; peptide nucleic acid (PNA) modified nucleotides; nucleotides with phosphorothioate linkages; a 5′ cap (e.g., a 7-methylguanylate cap (m7G)), phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkyl phosphoramidates, phosphorodiamidates, thionophosphor amidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage; phosphorothioate and/or heteroatom internucleoside linkages, such as —CH₂—NH—O—CH₂—, —CH₂—N(CH₃)—O—CH₂— (known as a methylene (methylimino) or MMI backbone), —CH₂—O—N(CH₃)—CH₂—, —CH₂—N(CH₃)—N(CH₃)—CH₂— and —O—N(CH₃)—CH₂—CH₂— (wherein the native phosphodiester internucleotide linkage is represented as —O—P(═O)(OH)—O—CH₂—); morpholino linkages (formed in part from the sugar portion of a nucleoside); morpholino backbones; phosphorodiamidate or other non-phosphodiester internucleoside linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; other backbone modifications having mixed N, O, S and CH₂ component parts; and combinations thereof.

### VII. Vectors and Multiplexed Expression Vectors

Compositions, systems, devices, kits, and methods described herein comprise a vector or a use thereof. A vector can comprise a nucleic acid of interest. In some embodiments, the nucleic acid of interest comprises one or more components of a composition or system described herein. In some embodiments, the nucleic acid of interest comprises a nucleotide sequence that encodes one or more components of the composition or system described herein. In some embodiments, one or more components comprises a polypeptide(s) (e.g., effector protein(s), effector partner(s), fusion partner(s), fusion protein(s), or combinations thereof), guide nucleic acid(s), target nucleic acid(s), and donor nucleic acid(s). In some embodiments, the component comprises a nucleic acid encoding a polypeptide (e.g., effector protein, effector partner, fusion partner, fusion protein, or combinations thereof), a donor nucleic acid, and a guide nucleic acid or a nucleic acid encoding the guide nucleic acid. In some embodiments, the vector is a part of a vector system. In some embodiments, the vector system comprises a library of vectors each encoding one or more component of a composition or system described herein. In some embodiments, components described herein (e.g., an effector protein, a guide nucleic acid, and/or a target nucleic acid) are encoded by the same vector. In some embodiments, components described herein (e.g., an effector protein, a guide nucleic acid, and/or a target nucleic acid) are each encoded by different vectors of the system. In some embodiments, a vector encoding a donor nucleic acid further encodes a target nucleic acid.

In some embodiments, a vector comprises a nucleotide sequence encoding one or more polypeptides (e.g., effector proteins, effector partners, fusion partners, fusion proteins, or combinations thereof) as described herein. In some embodiments, the one or more polypeptides comprise at least two polypeptides. In some embodiments, the at least two polypeptides are the same. In some embodiments, the at least two polypeptides are different from each other. In some embodiments, the nucleotide sequence is operably linked to a promoter that is operable in a target cell, such as a eukaryotic cell. In some embodiments, the vector comprises the nucleotide sequence encoding 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more polypeptides.

In some embodiments, a vector encodes one or more of any system components, including but not limited to polypeptide (e.g., effector protein, effector partner, fusion partner, fusion protein, or combinations thereof), guide nucleic acids, donor nucleic acids, and target nucleic acids as described herein. In some embodiments, a system component encoding sequence is operably linked to a promoter that is operable in a target cell, such as a eukaryotic cell. In some embodiments, a vector encodes 1, 2, 3, 4 or more of any system components. For example, in some embodiments, a vector encodes two or more guide nucleic acids, wherein each guide nucleic acid comprises a different sequence. In some embodiments, a vector encodes the polypeptide and the guide nucleic acid. In some embodiments, a vector encodes a polypeptide, a guide nucleic acid, a donor nucleic acid, or combinations thereof.

In some embodiments, a vector comprises one or more guide nucleic acids, or a nucleotide sequence encoding the one or more guide nucleic acids as described herein. In some embodiments, the one or more guide nucleic acids comprise at least two guide nucleic acids. In some embodiments, the at least two guide nucleic acids are the same. In some embodiments, the at least two guide nucleic acids are different from each other. In some embodiments, the guide nucleic acid or the nucleotide sequence encoding the guide nucleic acid is operably linked to a promoter that is operable in a target cell, such as a eukaryotic cell. In some embodiments, the vector comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more guide nucleic acids. In some embodiments, the vector comprises a nucleotide sequence encoding 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more guide nucleic acids.

In some embodiments, a vector comprises one or more donor nucleic acids as described herein. In some embodiments, the one or more donor nucleic acids comprise at least two donor nucleic acids. In some embodiments, the at least two donor nucleic acids are the same. In some embodiments, the at least two donor nucleic acids are different from each other. In some embodiments, the vector comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more donor nucleic acids.

In some embodiments, a vector comprises or encodes one or more regulatory elements. Regulatory elements, in some embodiments, are referred to as transcriptional and translational control sequences, such as promoters, enhancers, polyadenylation signals, terminators, and protein degradation signals, that provide for and/or regulate transcription of a non-coding sequence or a coding sequence and/or regulate translation of an encoded polypeptide. In some embodiments, a vector comprises or encodes for one or more additional elements, such as, for example, replication origins, antibiotic resistance (or a nucleic acid encoding the same), a tag (or a nucleic acid encoding the same), and selectable markers. In some embodiments, a vector comprises or encodes for one or more elements, such as, for example, ribosome binding sites, and RNA splice sites.

Vectors described herein can encode a promoter—a regulatory region on a nucleic acid, such as a DNA sequence, that initiate transcription of a downstream (3′ direction) coding or non-coding sequence. A promoter can be linked at its 3′ terminus to a nucleic acid, the expression or transcription of which is desired, and extends upstream (5′ direction) to include bases or elements necessary to initiate transcription or induce expression, which could be measured at a detectable level. A promoter can comprise a nucleotide sequence, referred to herein as a “promoter sequence”. The promoter sequence can include a transcription initiation site, and one or more protein binding domains responsible for the binding of transcription machinery, such as RNA polymerase. When eukaryotic promoters are used, such promoters can contain “TATA” boxes and “CAT” boxes. In some embodiments, various promoters, including inducible promoters, are used to drive expression, i.e., transcriptional activation, of the nucleic acid of interest. Accordingly, in some embodiments, the nucleic acid of interest can be operably linked to a promoter.

In some embodiments, promotors comprise any suitable type of promoter envisioned for the compositions, systems, and methods described herein. Examples include constitutively active promoters (e.g., CMV promoter), inducible promoters (e.g., heat shock promoter, tetracycline-regulated promoter, steroid-regulated promoter, metal-regulated promoter, estrogen receptor-regulated promoter, etc.), spatially restricted and/or temporally restricted promoters (e.g., a tissue specific promoter, a cell type specific promoter, etc.), etc. Suitable promoters include, but are not limited to: SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6), an enhanced U6 promoter, and a human Hl promoter (Hl). By transcriptional activation, it is intended that transcription will be increased above basal levels in the target cell by 2 fold, 5 fold, 10 fold, 50 fold, by 100 fold, 500 fold, or by 1000 fold, or more. In addition, vectors used for providing a nucleic acid that, when transcribed, produces a guide nucleic acid and/or a nucleic acid that encodes a polypeptide (e.g., effector protein, effector partner, fusion partner, fusion protein, or combinations thereof) to a cell comprising nucleic acid sequences that encode for selectable markers in the target cells, so as to identify cells that have taken up the guide nucleic acid and/or the polypeptides.

In general, vectors provided herein comprise at least one promotor or a combination of promoters driving expression or transcription of one or more genome editing tools described herein. In some embodiments, the vector comprises a nucleotide sequence of a promoter. In some embodiments, the vector comprises two promoters. In some embodiments, the vector comprises three promoters. In some embodiments, a length of the promoter is less than about 500, less than about 400, less than about 300, or less than about 200 linked nucleotides. In some embodiments, a length of the promoter is at least 100, at least 200, at least 300, at least 400, or at least 500 linked nucleotides. Non-limiting examples of promoters include CMV, 7SK, EF1a, RPBSA, hPGK, EFS, SV40, PGK1, Ubc, human beta actin, CAG, TRE, UAS, Ac5, Polyhedrin, CaMKIIa, GAL1-10, H1, TEF1, GDS, ADH1, CaMV35S, HSV TK, Ubi, U6, MNDU3, MSCV, MND, and CAG.

In some embodiments, some promoters (e.g., U6, enhanced U6, Hl and 7SK) prefer the nucleic acid being transcribed having “g” nucleotide at the 5′ end of the coding sequence. Accordingly, when such coding sequence is expressed, it comprises an additional “g” nucleotide at 5′ end. In some embodiments, vectors provided herein comprise a promotor driving expression or transcription of any one of the guide nucleic acids described herein. In some embodiments, the promotor is selected from U6, enhanced U6, Hl and 7SK.

In some embodiments, the promoter is a constitutive promoter. In some embodiments, the promoter is an inducible promoter. In some embodiments, the inducible promoter only drives expression of its corresponding coding sequence (e.g., polypeptide or guide nucleic acid) when a signal is present, e.g., a hormone, a small molecule, a peptide. Non-limiting examples of inducible promoters are the T7 RNA polymerase promoter, the T3 RNA polymerase promoter, the Isopropyl-beta-D-thiogalactopyranoside (IPTG)-regulated promoter, a lactose induced promoter, a heat shock promoter, a tetracycline-regulated promoter (tetracycline-inducible or tetracycline-repressible), a steroid regulated promoter, a metal-regulated promoter, and an estrogen receptor-regulated promoter. In some embodiments, the promoter is an activation-inducible promoter, such as a CD69 promoter. In some embodiments, the promoter for expressing a polypeptide (e.g., effector protein, effector partner, fusion partner, fusion protein, or combinations thereof) is a ubiquitous promoter. In some embodiments, the ubiquitous promoter comprises MND or CAG promoter sequence.

In some embodiments, the promoters are prokaryotic promoters (e.g., drive expression of a gene in a prokaryotic cell). In some embodiments, the promoters are eukaryotic promoters, (e.g., drive expression of a gene in a eukaryotic cell). In some embodiments, the promoter is EF1a. In some embodiments, the promoter is ubiquitin. In some embodiments, vectors are bicistronic or polycistronic vector (e.g., having or involving two or more loci responsible for generating a protein) having an internal ribosome entry site (IRES) is for translation initiation in a cap-independent manner.

In some embodiments, a vector described herein is a nucleic acid expression vector. In some embodiments, a vector described herein is a recombinant expression vector. In some embodiments, a vector described herein is a messenger RNA. In some embodiments, a vector comprising the recombinant nucleic acid as described herein, wherein the vector is a viral vector, an adeno associated viral (AAV) vector, a retroviral vector, or a lentiviral vector. In some embodiments, a vector described herein or a recombinant nucleic acid described herein is comprised in a cell. In some embodiments, a recombinant nucleic acid integrated into a genomic DNA sequence of the cell, wherein the cell is a eukaryotic cell or a prokaryotic cell. In some embodiments, the nucleic acid expression vector encodes at least one engineered guide nucleic acid.

In some embodiments, a vector described herein is a nucleic acid expression vector. In some embodiments, a nucleic acid expression vector as described herein is also described as a recombinant nucleic acid. In some embodiments, the recombinant nucleic acid encoding an effector protein comprises an amino acid sequence that is at least 85% identical to any one of the amino acid sequences set forth in TABLE 1 or TABLE 1.2. In some embodiments, the recombinant nucleic acid encoding the effector protein is operably linked to a promoter, wherein the promoter is functional in an eukaryotic cell or a prokaryotic cell. In some embodiments, the promoter is any one or more of: a constitutive promoter, an inducible promoter, a cell type-specific promoter, and a tissue-specific promoter. In some embodiments, the recombinant nucleic acid described herein wherein the promoter is functional in any one of: a plant cell, a fungal cell, an animal cell, cell of an invertebrate, a fly cell, a cell of a vertebrate, a mammalian cell, a primate cell, a non-human primate cell, and a human cell.

In some embodiments, a vector described herein is a delivery vector. In some embodiments, the delivery vector is a eukaryotic vector, a prokaryotic vector (e.g., a bacterial vector) a viral vector, or any combination thereof. In some embodiments, the delivery vehicle is a non-viral vector. In some embodiments, the delivery vector is a plasmid. In some embodiments, the plasmid comprises DNA. In some embodiments, the plasmid comprises RNA. In some embodiments, the plasmid comprises circular double-stranded DNA. In some embodiments, the plasmid is linear. In some embodiments, the plasmid comprises one or more coding sequences of interest and one or more regulatory elements. In some embodiments, the plasmid comprises a bacterial backbone containing an origin of replication and an antibiotic resistance gene or other selectable marker for plasmid amplification in bacteria. In some embodiments, the plasmid is a minicircle plasmid. In some embodiments, the plasmid contains one or more genes that provide a selective marker to induce a target cell to retain the plasmid. In some examples, the plasmid is formulated for delivery through injection by a needle carrying syringe. In some examples, the plasmid is formulated for delivery via electroporation. In some examples, the plasmids are engineered through synthetic or other suitable means known in the art. For example, in some embodiments, the genetic elements are assembled by restriction digest of the desired genetic sequence from a donor plasmid or organism to produce ends of the DNA which is then be readily ligated to another genetic sequence.

In some embodiments, vectors comprise an enhancer. Enhancers are nucleotide sequences that have the effect of enhancing promoter activity. In some embodiments, enhancers augment transcription regardless of the orientation of their sequence. In some embodiments, enhancers activate transcription from a distance of several kilo basepairs. Furthermore, enhancers are located optionally upstream or downstream of a gene region to be transcribed, and/or located within the gene, to activate the transcription. Exemplary enhancers include, but are not limited to, WPRE; CMV enhancers; the R-U5′ segment in LTR of HTLV-I.

### Administration of a Non-Viral Vector

In some embodiments, an administration of a non-viral vector comprises contacting a cell, such as a host cell, with the non-viral vector. In some embodiments, a physical method or a chemical method is employed for delivering the vector into the cell. Exemplary physical methods include electroporation, gene gun, sonoporation, magnetofection, or hydrodynamic delivery. Exemplary chemical methods include delivery of the recombinant polynucleotide by liposomes such as, cationic lipids or neutral lipids; lipofection; dendrimers; lipid nanoparticle (LNP); or cell-penetrating peptides.

In some embodiments, a vector is administered as part of a method of nucleic acid detection, modifying, and/or treatment as described herein. In some embodiments, a vector is administered in a single vehicle, such as a single expression vector. In some embodiments, at least two of the three components, a nucleic acid encoding one or more polypeptides (e.g., effector proteins, effector partners, fusion partners, fusion proteins, or combinations thereof), one or more donor nucleic acids, and one or more guide nucleic acids or a nucleic acid encoding the one or more guide nucleic acid, are provided in the single expression vector. In some embodiments, components, such as a guide nucleic acid and a polypeptide (e.g., effector protein, effector partner, fusion partner, fusion protein, or combinations thereof), are encoded by the same vector. In some embodiments, a polypeptide (e.g., effector protein, effector partner, fusion partner, fusion protein, or combinations thereof) (or a nucleic acid encoding same) and/or an engineered guide nucleic acid (or a nucleic acid that, when transcribed, produces same) are not co-administered with donor nucleic acid in a single vehicle. In some embodiments, a polypeptide (e.g., effector protein, effector partner, fusion partner, fusion protein, or combinations thereof) (or a nucleic acid encoding same), an engineered guide nucleic acid (or a nucleic acid that, when transcribed, produces same), and/or donor nucleic acid are administered in one or more or two or more vehicles, such as one or more, or two or more expression vectors.

In some embodiments, a vector may be part of a vector system. In some embodiments, the vector system comprises a library of vectors each encoding one or more components of a composition or system described herein. In some embodiments, a vector system is administered as part of a method of nucleic acid detection, modifying, and/or treatment as described herein, wherein at least two vectors are co-administered. In some embodiments, the at least two vectors comprise different components. In some embodiments, the at least two vectors comprise the same component having different sequences. In some embodiments, at least one of the three components, a nucleic acid encoding one or more polypeptides (e.g., effector proteins, effector partners, fusion partners, fusion proteins, or combinations thereof), one or more donor nucleic acids, and one or more guide nucleic acids or a nucleic acid encoding the one or more guide nucleic acids, or a variant thereof is provided in a different vector. In some embodiments, the nucleic acid encoding the polypeptides (e.g., effector proteins, effector partners, fusion partners, fusion proteins, or combinations thereof), and a guide nucleic acid or a nucleic acid encoding the guide nucleic acid are provided in different vectors. In some embodiments, the donor nucleic acid is encoded by a different vector than the vector encoding the effector protein and the guide nucleic acid.

### Lipid Particles and Non-Viral Vectors

In some embodiments, compositions, systems, devices, and kits provided herein comprise a lipid or lipid particle. In some embodiments, a lipid particle is a lipid nanoparticle (LNP). In some embodiments, a lipid or a lipid nanoparticle can encapsulate a nucleic acid (e.g., DNA or RNA) encoding one or more of the components as described herein. In some embodiments, a lipid or a lipid nanoparticle can encapsulate an expression vector as described herein. LNPs are a non-viral delivery system for delivery of the composition and/or system components described herein. LNPs are particularly effective for delivery of nucleic acids. Beneficial properties of LNP include ease of manufacture, low cytotoxicity and immunogenicity, high efficiency of nucleic acid encapsulation and cell transfection, multi-dosing capabilities and flexibility of design (Kulkarni et al., (2018) Nucleic Acid Therapeutics, 28(3):146-157). In some embodiments, compositions and methods comprise a lipid, polymer, nanoparticle, or a combination thereof, or use thereof, to introduce one or more effector proteins, one or more guide nucleic acids, one or more donor nucleic acids, or any combinations thereof to a cell. Non-limiting examples of lipids and polymers are cationic polymers, cationic lipids, ionizable lipids, or bio-responsive polymers. In some embodiments, the ionizable lipids exploits chemical-physical properties of the endosomal environment (e.g., pH) offering improved delivery of nucleic acids. In some embodiments, the ionizable lipids are neutral at physiological pH. In some embodiments, the ionizable lipids are protonated under acidic pH. In some embodiments, the bio-responsive polymer exploits chemical-physical properties of the endosomal environment (e.g., pH) to preferentially release the genetic material in the intracellular space.

In some embodiments, a LNP comprises an outer shell and an inner core. In some embodiments, the outer shell comprises lipids. In some embodiments, the lipids comprise modified lipids. In some embodiments, the modified lipids comprise pegylated lipids. In some embodiments, the lipids comprise one or more of cationic lipids, anionic lipids, ionizable lipids, and non-ionic lipids. In some embodiments, the LNP comprises one or more of N1,N3,N5-tris(3-(didodecylamino)propyl)benzene-1,3,5-tricarboxamide (TT3), 1,2-Dioleoyl-sn-glycero-3-phosphocholine (DOPC), 2-dioleoyl-sn-glycero-3-phosphoethanolamine (DOPE), 1-palmitoyl-2-oleoylsn-glycero-3-phosphoethanolamine (POPE), 1,2-distearoyl-sn-glycero-3-phosphocholine (DSPC), cholesterol (Chol), 1,2-dimyristoyl-sn-glycerol, and methoxypolyethylene glycol (DMG-PEChooo), 1,2-dimyristoyl-rac-glycero-3-methoxypolyethylene glycol-2000 (DMG-PEG 2000), derivatives, analogs, or variants thereof, or any combination of the foregoing.

In some embodiments, the LNP comprises one or more ionizable lipid. Such ionizable lipids include, but are not limited to: 4-(dimethylamino)-butanoic acid, (10Z,13Z)-1-(9Z,12Z)-9,12-octadecadien-1-yl-10,13-nonadecadien-1-yl ester (DLin-MC3-DMA, CAS No. 1224606-06-7); N,N-dimethyl-2,2-di-(9Z,12Z)-9,12-octadecadien-1-yl-1,3-dioxolane-4-ethanamine (DLin-KC2-DMA, CAS No. 1190197-97-7); 8-[(2-hydroxyethyl)[6-oxo-6-(undecyloxy)hexyl]amino]-octanoic acid, 1-octylnonyl ester (SM-102, CAS No. 2089251-47-6); 8-[(2-hydroxyethyl)[8-(nonyloxy)-8-oxooctyl]amino]-octanoic acid, 1-octylnonyl ester (Lipid 5, CAS No. 2089251-33-0); 1,1′-[[2-[4-[2-[[2-[bis(2-hydroxydodecyl)amino]ethyl](2-hydroxydodecyl)amino]ethyl]-1-piperazinyl]ethyl]imino]bis-2-dodecanol (C12-200, CAS No. 1220890-25-4); 2-hexyl-decanoic acid, 1,1′-[[(4-hydroxybutyl)imino]di-6,1-hexanediyl]ester (ALC-0315, CAS No. 2036272-55-4); 9,12-octadecadienoic acid, (9Z,12Z)-1,1′,1″,1′″-[(3,6-dioxo-2,5-piperazinediyl)bis(4,1-butanediylnitrilodi-4,1-butanediyl)]ester (OF-C4-Deg-Lin, CAS No. 1853203-01-6); bis(2-(dodecyldisulfaneyl)ethyl) 3,3′-((3-methyl-9-oxo-10-oxa-13,14-dithia-3,6-diazahexacosyl)azanediyl)dipropionate (BAMEA-O16B, CAS No. 2490668-30-7); 3,6-bis[4-[bis[(9Z,12Z)-2-hydroxy-9,12-octadecadien-1-yl]amino]butyl]-2,5-piperazinedione (OF-02, CAS No. 1883431-67-1); tetrakis(8-methylnonyl) 3,3′,3″,3′″-(((methylazanediyl)bis(propane-3,1-diyl))bis(azanetriyl))tetrapropionate (3060i10, CAS No. 2322290-93-5); tetrakis(2-(octyldisulfaneyl)ethyl) 3,3′,3″,3′″-(((methylazanediyl)bis(propane-3,1-diyl))bis(azanetriyl))tetrapropionate (306-012B, CAS No. 2566523-06-4); bis(2-butyloctyl) 10-(N-(3-(dimethylamino)propyl)nonanamido)nonadecanedioate (Lipid A9, CAS No. 2036272-50-9); Arcturus Lipid 2,2 (8,8) 4C CH3 (ATX-0114, CAS No. 2230647-28-4)); di((Z)-non-2-en-1-yl) 8,8′-((2-((2-(dimethylamino)ethyl)thio)acetyl)azanediyl)dioctanoate (ATX-001, CAS No. 1777792-33-2); di((Z)-non-2-en-1-yl) 8,8′-((((2-(dimethylamino)ethyl)thio)carbonyl)azanediyl)dioctanoate (ATX-002, CAS No. 1777792-34-3); Genevant CL1 (CAS No. 1450888-71-7); LP01; hexa(octan-3-yl) 9,9′,9″,9′″,9″″,9′″″-((((benzene-1,3,5-tricarbonyl)yris(azanediyl)) tris (propane-3,1-diyl)) tris(azanetriyl))hexanonanoate (FTT5); 5A2-SC8 (CAS No. 1857341-90-2); COATSOME® SS-OP; derivatives; analogs; or variants thereof. In some embodiments, the LNP comprise a combination of two, three, four, five or more of the foregoing ionizable lipids.

In some embodiments, the LNP has a negative net overall charge prior to complexation with one or more of a guide nucleic acid, a nucleic acid encoding the one or more guide nucleic acid, a nucleic acid encoding the one or more polypeptide (e.g., one or more effector proteins, effector partners, fusion partners, fusion proteins, or combinations thereof), and/or a donor nucleic acid. In some embodiments, the inner core is a hydrophobic core. In some embodiments, the one or more of a guide nucleic acid, the nucleic acid encoding the one or more guide nucleic acid, the nucleic acid encoding the polypeptide, and/or the donor nucleic acid forms a complex with one or more of the cationic lipids and the ionizable lipids. In some embodiments, the nucleic acid encoding the polypeptide or the nucleic acid encoding the guide nucleic acid is self-replicating.

In some embodiments, a LNP comprises one or more of cationic lipids, ionizable lipids, and modified versions thereof. In some embodiments, the ionizable lipid comprises N1,N3,N5-tris(3-(didodecylamino)propyl)benzene-1,3,5-tricarboxamide (TT3) or a derivative thereof. Accordingly, in some embodiments, the LNP comprises one or more of T3 and pegylated TT3. The publication WO2016187531 is hereby incorporated by reference in its entirety, which describes representative LNP formulations in Table 2 and Table 3, and representative methods of delivering LNP formulations in Example 7.

In some embodiments, a LNP comprises a lipid composition targeting to a specific organ. In some embodiments, the lipid composition comprises lipids having a specific alkyl chain length that controls accumulation of the LNP in the specific organ (e.g., liver or spleen). In some embodiments, the lipid composition comprises a biomimetic lipid that controls accumulation of the LNP in the specific organ (e.g., brain). In some embodiments, the lipid composition comprises lipid derivatives (e.g., cholesterol derivatives) that controls accumulation of the LNP in a specific cell (e.g., liver endothelial cells, Kupffer cells, hepatocytes).

In some embodiments, the LNP described herein comprises nucleic acids (e.g., DNA or RNA) encoding an effector protein described herein, an effector partner described herein, a fusion protein described herein, a guide nucleic acid described herein, or combinations thereof. In some embodiments, the LNP comprises an mRNA that produces an effector protein described herein, an effector partner described herein, or a fusion protein described herein when translated. In some embodiments, the LNP comprises chemically modified guide nucleic acids.

### Delivery of Viral Vectors

In some embodiments, a vector described herein comprises a viral vector. In some embodiments, the viral vector comprises a nucleic acid to be delivered into a host cell by a recombinantly produced virus or viral particle. In some embodiments, the nucleic acid comprises single-stranded or double stranded, linear or circular, segmented or non-segmented. In some embodiments, the nucleic acid comprises DNA, RNA, or a combination thereof. In some embodiments, the vector is an adeno-associated viral vector. There are a variety of viral vectors that are associated with various types of viruses, including but not limited to retroviruses (e.g., lentiviruses and γ-retroviruses), adenoviruses, arenaviruses, alphaviruses, adeno-associated viruses (AAVs), baculoviruses, vaccinia viruses, herpes simplex viruses and poxviruses. In some embodiments, the vector is an adeno-associated viral (AAV) vector. In some embodiments, the viral vector is a recombinant viral vector. In some embodiments, the vector is a retroviral vector. In some embodiments, the retroviral vector is a lentiviral vector. In some embodiments, the retroviral vector comprises gamma-retroviral vector. In some embodiments, a viral vector provided herein is derived from or based on any such virus. For example, in some embodiments, the gamma-retroviral vector is derived from a Moloney Murine Leukemia Virus (MoMLV, MMLV, MuLV, or MLV) or a Murine Stem cell Virus (MSCV) genome. In some embodiments, the lentiviral vector is derived from the human immunodeficiency virus (HIV) genome. In some embodiments, the viral vector is a chimeric viral vector. In some embodiments, the chimeric viral vector comprises viral portions from two or more viruses. In some embodiments, the viral vector corresponds to a virus of a specific serotype.

In some embodiments, a viral vector is an adeno-associated viral vector (AAV vector). In some embodiments, a viral particle that delivers a viral vector described herein is an AAV. In some embodiments, the AAV comprises any AAV known in the art. In some embodiments, the viral vector corresponds to a virus of a specific AAV serotype. In some embodiments, the AAV serotype is selected from an AAV1 serotype, an AAV2 serotype, AAV3 serotype, an AAV4 serotype, AAV5 serotype, an AAV6 serotype, AAV7 serotype, an AAV8 serotype, an AAV9 serotype, an AAV10 serotype, an AAV11 serotype, an AAV12 serotype, an AAV-rh10 serotype, and any combination, derivative, or variant thereof. In some embodiments, the AAV vector is a recombinant vector, a hybrid AAV vector, a chimeric AAV vector, a self-complementary AAV (scAAV) vector, a single-stranded AAV, or any combination thereof. scAAV genomes are generally known in the art and contain both DNA strands which can anneal together to form double-stranded DNA.

In some embodiments, an AAV vector described herein is a chimeric AAV vector. In some embodiments, the chimeric AAV vector comprises an exogenous amino acid or an amino acid substitution, or capsid proteins from two or more serotypes. In some embodiments, a chimeric AAV vector is genetically engineered to increase transduction efficiency, selectivity, or a combination thereof.

In some embodiments, AAV vector described herein comprises two inverted terminal repeats (ITRs). According, in some embodiments, the viral vector provided herein comprises two inverted terminal repeats of AAV. A nucleotide sequence between the ITRs of an AAV vector provided herein comprises a sequence encoding genome editing tools. In some embodiments, the genome editing tools comprise a nucleic acid encoding one or more polypeptides (e.g., effector proteins, effector partners, fusion partners, fusion proteins, or combinations thereof), a nucleic acid encoding the one or more fusion proteins or polypeptides comprising a heterologous peptide (e.g., a nuclear localization signal (NLS), polyA tail), one or more guide nucleic acids, a nucleic acid encoding the one or more guide nucleic acids, respective promoter(s), one or more donor nucleic acid, or any combinations thereof. In some embodiments, viral vectors provided herein comprise at least one promotor or a combination of promoters driving expression or transcription of one or more genome editing tools described herein. In some embodiments, a coding region of the AAV vector forms an intramolecular double-stranded DNA template thereby generating the AAV vector that is a self-complementary AAV (scAAV) vector. In some embodiments, the scAAV vector comprises the nucleotide sequence encoding genome editing tools that has a length of about 2 kb to about 3 kb. In some embodiments, the AAV vector provided herein is a self-inactivating AAV vector. In some embodiments, the AAV vector provided herein comprises a modification, such as an insertion, deletion, chemical alteration, or synthetic modification, relative to a wild-type AAV vector.

### Producing AAV Delivery Vectors

In some embodiments, methods of producing AAV delivery vectors herein comprise packaging a nucleic acid encoding a polypeptide (e.g., effector protein, effector partner, fusion partner, fusion protein, or combinations thereof) and a guide nucleic acid, or a combination thereof, into an AAV vector. In some embodiments, methods of producing the delivery vector comprises, (a) contacting a cell with at least one nucleic acid encoding: (i) a guide nucleic acid; (ii) a Replication (Rep) gene; and (iii) a Capsid (Cap) gene that encodes an AAV capsid protein; (b) expressing the AAV capsid protein in the cell; (c) assembling an AAV particle; and (d) packaging the polypeptide encoding nucleic acid into the AAV particle, thereby generating an AAV delivery vector. In some embodiments, promoters, stuffer sequences, and any combination thereof are packaged in the AAV vector. In some embodiments, the AAV vector packages 1, 2, 3, 4, or 5 guide nucleic acids or copies thereof. In some embodiments, the AAV vector comprises inverted terminal repeats, e.g., a 5′ inverted terminal repeat and a 3′ inverted terminal repeat. In some embodiments, the AAV vector comprises a mutated inverted terminal repeat that lacks a terminal resolution site.

In some embodiments, a hybrid AAV vector is produced by transcapsidation, e.g., packaging an inverted terminal repeat (ITR) from a first serotype into a capsid of a second serotype, wherein the first and second serotypes are not the same. In some examples, the Rep gene and ITR from a first AAV serotype (e.g., AAV2) is used in a capsid from a second AAV serotype (e.g., AAV9), wherein the first and second AAV serotypes are not the same. As a non-limiting example, a hybrid AAV serotype comprising the AAV2 ITRs and AAV9 capsid protein is indicated AAV2/9. In some examples, the hybrid AAV delivery vector comprises an AAV2/1, AAV2/2, AAV 2/4, AAV2/5, AAV2/8, or AAV2/9 vector.

### Producing AAV Particles

In some embodiments, AAV particles described herein are recombinant AAV (rAAV). In some embodiments, rAAV particles are generated by transfecting AAV producing cells with an AAV-containing plasmid carrying the nucleotide sequence encoding the genome editing tools, a plasmid that carries viral encoding regions, i.e., Rep and Cap gene regions; and a plasmid that provides the helper genes such as E1A, E1B, E2A, E40RF6 and VA. In some embodiments, the AAV producing cells are mammalian cells. In some embodiments, host cells for rAAV viral particle production are mammalian cells. In some embodiments, a mammalian cell for rAAV viral particle production is a COS cell, a HEK293T cell, a HeLa cell, a KB cell, a variant thereof, or a combination thereof. In some embodiments, rAAV virus particles can be produced in the mammalian cell culture system by providing the rAAV plasmid to the mammalian cell. In some embodiments, producing rAAV virus particles in a mammalian cell comprises transfecting vectors that express the rep protein, the capsid protein, and the gene-of-interest expression construct flanked by the ITR sequence on the 5′ and 3′ ends. Methods of such processes are provided in, for example, Naso et al., BioDrugs, 2017 August; 31(4):317-334 and Benskey et al., (2019), Methods Mol Biol., 1937:3-26, each of which is incorporated by reference in their entireties.

In some embodiments, rAAV is produced in a non-mammalian cell. In some embodiments, rAAV is produced in an insect cell. In some embodiments, the insect cell for producing rAAV viral particles comprises a Sf9 cell. In some embodiments, production of rAAV virus particles in insect cells comprises infecting the insect cells with baculovirus. In some embodiments, production of rAAV virus particles in insect cells comprises infecting the insect cells with three recombinant baculoviruses, one carrying the cap gene, one carrying the rep gene, and one carrying the gene-of-interest expression construct enclosed by an ITR on both the 5′ and 3′ end. In some embodiments, rAAV virus particles are produced by the One Bac system. In some embodiments, rAAV virus particles can be produced by the Two Bac system. In some embodiments, in the Two Bac system, the rep gene and the cap gene of the AAV is integrated into one baculovirus virus genome, and the ITR sequence and the gene-of-interest expression construct is integrated into another baculovirus virus genome. In some embodiments, in the One Bac system, an insect cell line that expresses both the rep protein and the capsid protein is established and infected with a baculovirus virus integrated with the ITR sequence and the gene-of-interest expression construct. Details of such processes are provided in, for example, Smith et. al., (1983), Mol. Cell. Biol., 3(12):2156-65; Urabe et al., (2002), Hum. Gene. Ther., 1; 13(16):193543; and Benskey et al., (2019), Methods Mol Biol., 1937:3-26, each of which is incorporated by reference in its entirety.

### VIII. Target Nucleic Acids

Disclosed herein are compositions, systems, devices, kits, and methods for detecting and/or modifying a target nucleic acid. In some embodiments, the target nucleic acid is a double stranded nucleic acid. In some embodiments, the target nucleic acid is a single stranded nucleic acid. Alternatively, or in combination, the target nucleic acid is a double stranded nucleic acid and is prepared into single stranded nucleic acids before or upon contacting an RNP. In some embodiments, the single stranded nucleic acid comprises a RNA, wherein the RNA comprises a mRNA, a circular RNA, a rRNA, a tRNA, a non-coding RNA, a long non-coding RNA, a microRNA (miRNA), and a single-stranded RNA (ssRNA). In some embodiments, the target nucleic acid is a single-stranded RNA template from which complementary DNA (cDNA) is synthesized from in a reaction catalyzed by a reverse transcriptase. In some embodiments, the target nucleic acid comprises RNA, DNA, or combinations thereof. In some embodiments, the target nucleic acid is a mRNA. In some embodiments, the target nucleic acid is a long non-coding RNA. In some embodiments, the target nucleic acid is circular RNA. In some embodiments, the target nucleic acid is a long non-coding RNA or a miRNA. In some embodiments, the target nucleic acid is single stranded RNA, double stranded RNA, linear single-stranded RNA, circular RNA, coding RNA, non-coding RNA, or combinations thereof. In some embodiments, guide nucleic acids described herein hybridize to a portion of the target nucleic acid. In some embodiments, the target nucleic acid is from a virus, a parasite, or a bacterium described herein.

In some embodiments, a target nucleic acid comprises 5 to 100, 5 to 90, 5 to 80, 5 to 70, 5 to 60, 5 to 50, 5 to 40, 5 to 30, 5 to 25, 5 to 20, 5 to 15, or 5 to 10 linked nucleotides. In some embodiments, the target nucleic acid comprises 10 to 90, 20 to 80, 30 to 70, or 40 to 60 linked nucleotides. In some embodiments, the target nucleic acid comprises 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 60, 70, 80, 90, or 100 linked nucleotides. In some embodiments, the target nucleic acid comprises at least 10, at least 20, at least 30, at least 40, at least 50, at least 60, at least 70, at least 80, at least 90, or at least 100 linked nucleotides. In some embodiments, the target sequence in the target nucleic acid comprises at least 10 contiguous nucleotides that are complementary to the guide nucleic acid or engineered guide nucleic acid.

In some embodiments, compositions, systems, devices, kits, and methods described herein comprise a target nucleic acid that is responsible for a disease, contain a mutation (e.g., single strand polymorphism, point mutation, insertion, or deletion), be contained in an amplicon, or be uniquely identifiable from the surrounding nucleic acids (e.g., contain a unique sequence of nucleotides). In some embodiments, the target nucleic acid has undergone a modification (e.g., an editing) after contacting with an RNP. In some embodiments, the modifying is a change in the nucleotide sequence of the target nucleic acid. In some embodiments, the change comprises an insertion, deletion, or substitution of one or more nucleotides compared to the target nucleic acid that has not undergone any modification.

In some embodiments, the target nucleic acid comprises a nucleic acid sequence from a pathogen responsible for a disease. Non-limiting examples of pathogens are bacteria, a virus and a fungus. In some embodiments, the target sequence is a portion of a nucleic acid from a virus or a bacterium or other agents responsible for a disease in the sample. The target sequence, in some embodiments, is a portion of a nucleic acid from a sexually transmitted infection or a contagious disease, in the sample. The target sequence, in some embodiments, is a portion of a nucleic acid from an upper respiratory tract infection, a lower respiratory tract infection, or a contagious disease, in the sample. The target sequence, in some embodiments, is a portion of a nucleic acid from a hospital acquired infection or a contagious disease, in the sample. The target sequence, in some embodiments, is a portion of a nucleic acid from sepsis, in the sample. In some embodiments, the target nucleic acid is a portion of a nucleic acid associated with a genomic locus, a nucleic acid associated with any DNA amplicon or a cDNA, such as a mRNA, a circular RNA, or any RNA associated with a gene locus in at least one of: human immunodeficiency virus (HIV), human papillomavirus (HPV), chlamydia, gonorrhea, syphilis, trichomoniasis, sexually transmitted infection, malaria, Dengue fever, Ebola, chikungunya, and leishmaniasis. Pathogens include viruses, fungi, helminths, protozoa, malarial parasites, Plasmodium parasites, Toxoplasma parasites, and Schistosoma parasites. Helminths include roundworms, heartworms, and phytophagous nematodes, flukes, Acanthocephala, and tapeworms. Protozoan infections include infections from Giardia spp., Trichomonas spp., African trypanosomiasis, amoebic dysentery, babesiosis, balantidial dysentery, Chaga's disease, coccidiosis, malaria and toxoplasmosis. Examples of pathogens such as parasitic/protozoan pathogens include, but are not limited to: Plasmodium falciparum, P. vivax, Trypanosoma cruzi and Toxoplasma gondii. Fungal pathogens include, but are not limited to Cryptococcus neoformans, Histoplasma capsulatum, Coccidioides immitis, Blastomyces dermatitidis, Chlamydia trachomatis, and Candida albicans. A pathogenic virus can be a DNA virus or an RNA virus. Pathogenic viruses include but are not limited to respiratory viruses, adenoviruses, parainfluenza viruses, severe acute respiratory syndrome (SARS), coronavirus (e.g., SARS-CoV), MERS, gastrointestinal viruses (e.g., noroviruses, rotaviruses, some adenoviruses, astroviruses), exanthematous viruses (e.g., the virus that causes measles, the virus that causes rubella, the virus that causes chickenpox/shingles, the virus that causes roseola, the virus that causes smallpox, the virus that causes fifth disease, chikungunya virus infection); hepatic viral diseases (e.g., hepatitis A, B, C, D, E); cutaneous viral diseases (e.g., warts (including genital, anal), herpes (including oral, genital, anal), molluscum contagiosum); hemmorhagic viral diseases (e.g., Ebola, Lassa fever, dengue fever, yellow fever, Marburg hemorrhagic fever, Crimean-Congo hemorrhagic fever); neurologic viruses (e.g., polio, viral meningitis, viral encephalitis, rabies), sexually transmitted viruses (e.g., HIV, HPV, and the like), Adenovirus, coronavirus (i.e., a virus that causes COVID-19), Coronavirus HKU1, Coronavirus NL63, Coronavirus 229E, Coronavirus OC43, Severe Acute Respiratory Syndrome Coronavirus 2 (SARS-CoV-2), Human Metapneumovirus (hMPV), Human Rhinovirus/Enterovirus, influenza virus, Influenza A, Influenza A/H1, Influenza A/H3, Influenza A/H1-2009, Influenza B, Influenza C, Parainfluenza Virus 1, Parainfluenza Virus 2, Parainfluenza Virus 3, Parainfluenza Virus 4, Respiratory Syncytial Virus), human immunodeficiency virus (e.g., HIV); human papillomavirus (e.g., HPV), chlamydia, gonorrhea, syphilis, trichomoniasis, sexually transmitted infection, malaria, Dengue fever, Ebola, chikungunya, and leishmaniasis; Orthopoxvirus (e.g., monkeypox virus, cowpox virus, camelpox virus, horsepox virus, vaccinia virus, and variola virus); West Nile virus, herpes virus, yellow fever virus, Hepatitis Virus C, Hepatitis Virus A, Hepatitis Virus B, and papillomavirus. Pathogens include, e.g., HIV virus, Bordetella parapertussis, Bordetella pertussis, Chlamydia pneumoniae, Mycoplasma pneumoniae, Mycobacterium tuberculosis, Streptococcus agalactiae, methicillin-resistant Staphylococcus aureus, Legionella pneumophila, Streptococcus pyogenes, Escherichia coli, Neisseria gonorrhoeae, Neisseria meningitidis, Pneumococcus, Cryptococcus neoformans, Histoplasma capsulatum, Hemophilus influenzae B, Treponema pallidum, Lyme disease spirochetes, Pseudomonas aeruginosa, Mycobacterium leprae, Brucella abortus, rabies virus, influenza virus, cytomegalovirus, herpes simplex virus I, herpes simplex virus II, human serum parvo-like virus, respiratory syncytial virus (RSV), M. genitalium, T. vaginalis, varicella-zoster virus, hepatitis B virus, hepatitis C virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, Reovirus, polio virus, simian virus 40, mouse mammary tumor virus, dengue virus, rubella virus, West Nile virus, Plasmodium falciparum, Plasmodium vivax, Toxoplasma gondii, Trypanosoma rangeli, Trypanosoma cruzi, Trypanosoma rhodesiense, Trypanosoma brucei, Schistosoma mansoni, Schistosoma japonicum, Babesia bovis, Eimeria tenella, Onchocerca volvulus, Leishmania tropica, Mycobacterium tuberculosis, Trichinella spiralis, Theileria parva, Taenia hydatigena, Taenia ovis, Taenia saginata, Echinococcus granulosus, Mesocestoides corti, Mycoplasma arthritidis, M. hyorhinis, M. orale, M. arginini, Acholeplasma laidlawii, M. salivarium, M. pneumoniae, Enterobacter cloacae, Kiebsiella aerogenes, Proteus vulgaris, Serratia macesens, Enterococcus faecalis, Enterococcus faecium, Streptococcus intermdius, Streptococcus pneumoniae, and Streptococcus pyogenes. In some embodiments, the target sequence is a portion of a nucleic acid associated with a genomic locus, a mRNA, or a nucleic acid associated with a cDNA from a gene locus of bacterium or other agents responsible for a disease in the sample comprising a mutation that confers resistance to a treatment, such as a single nucleotide mutation that confers resistance to antibiotic treatment.

In some embodiments, the target sequence is comprised in a sample. In some embodiments, the sample used for genetic disorder testing, cancer testing, or cancer risk testing can comprise at least one target sequence or target nucleic acid segment that can bind to a guide nucleic acid of the reagents described herein. In some embodiments, the sample used comprises a target sequence or target nucleic acid of a gene recited in TABLE 4 and TABLE 4.1.

In some embodiments, the sample used for phenotyping testing can comprise at least one target nucleic acid segment that can bind to a guide nucleic acid of the reagents described herein. The target nucleic acid segment, in some cases, is a portion of a nucleic acid from a gene associated with a phenotypic trait.

In some embodiments, the sample used for genotyping testing can comprise at least one target nucleic acid segment that can bind to a guide nucleic acid of the reagents described herein. The target nucleic acid segment, in some cases, is a portion of a nucleic acid from a gene associated with a genotype.

In some embodiments, the target nucleic acid comprises a nucleic acid sequence of a virus, a bacterium, or other pathogen responsible for a disease in a plant (e.g., a crop). In some embodiments, methods and compositions of the disclosure are used for treating or detecting a disease in a plant. For example, in some embodiments, the methods of the disclosure are used for targeting a viral nucleic acid sequence in a plant. In some embodiments, an effector protein of the disclosure cleaves the viral nucleic acid. In some embodiments, the target nucleic acid comprises a nucleic acid sequence of a virus or a bacterium or other agents (e.g., any pathogen) responsible for a disease in the plant (e.g., a crop). The target nucleic acid, in some embodiments, is a portion of a nucleic acid from a virus or a bacterium or other agents responsible for a disease in the plant (e.g., a crop). In some embodiments, the target nucleic acid comprises RNA, a portion of a nucleic acid associated with a genomic locus, a nucleic acid associated with any DNA amplicon or a cDNA, such as a mRNA, a circular RNA, or any RNA associated with a gene locus in at a virus or a bacterium or other agents (e.g., any pathogen) responsible for a disease in the plant (e.g., a crop). In some embodiments, a virus infecting the plant comprises an RNA virus. In some embodiments, a virus infecting the plant comprises a DNA virus. Non-limiting examples of viruses that may be targeted with the disclosure include Tobacco mosaic virus (TMV), Tomato spotted wilt virus (TSWV), Cucumber mosaic virus (CMV), Potato virus Y (PVY), Cauliflower mosaic virus (CaMV) (RT virus), Plum pox virus (PPV), Brome mosaic virus (BMV) and Potato virus X (PVX).

In some embodiments, a target nucleic acid comprises a portion or a specific region of a nucleic acid associated with a genomic locus, a nucleic acid associated with any DNA amplicon or a cDNA, such as a mRNA, a circular RNA, or any RNA associated with a gene described herein. In some embodiments, the target nucleic acid is an amplicon of at least a portion of a nucleic acid associated with a gene. Non-limiting examples of genes are recited in TABLE 4 and TABLE 4.1. Nucleic acid sequences of target nucleic acids and/or corresponding genes are readily available in public databases as known and used in the art. In some embodiments, the target nucleic acid is selected from TABLE 4 and TABLE 4.1. In some embodiments, the target nucleic acid comprises one or more target sequences. In some embodiments, the one or more target sequence is within any one of the target nucleic acids set forth in TABLE 4 and TABLE 4.1.

In some embodiments, the target nucleic acid is any one of: a naturally occurring eukaryotic sequence, a naturally occurring prokaryotic sequence, a naturally occurring viral sequence, a naturally occurring bacterial sequence, a naturally occurring fungal sequence, an engineered eukaryotic sequence, an engineered prokaryotic sequence, an engineered viral sequence, an engineered bacterial sequence, an engineered fungal sequence, a fragment of a naturally occurring sequence, a fragment of an engineered sequence, and combinations thereof.

In some embodiments, the target nucleic acid is isolated from any one of: a naturally occurring cell, a eukaryotic cell, a prokaryotic cell, a plant cell, a fungal cell, an animal cell, cell of an invertebrate, a fly cell, a cell of a vertebrate, a mammalian cell, a primate cell, a non-human primate cell, a human cell, a living cell, a non-living cell, a modified cell, a derived cell, and a non-naturally occurring cell. In some embodiments, the target nucleic acid is isolated from a population of cells.

In some embodiments, a precursor messenger RNA (pre-mRNA) transcript is processed by RNA splicing, wherein RNA splicing comprises removal of all introns (non-coding regions of RNA) and splicing back together exons (coding regions of RNA), producing the target nucleic acid, wherein the target nucleic acid is a mature messenger RNA (mRNA). Nucleic acids, such as pre-mRNA described herein, can contain at least one intron and at least one exon, wherein as read in the 5′ to the 3′ direction of a nucleic acid strand, the 3′ end of an intron can be adjacent to the 5′ end of an exon, and wherein said intron and exon correspond for transcription purposes. If a nucleic acid strand contains more than one intron and exon, the 5′ end of the second intron is adjacent to the 3′ end of the first exon, and 5′ end of the second exon is adjacent to the 3′ end of the second intron. The junction between an intron and an exon can be referred to herein as a splice junction, wherein a 5′ splice site (SS) can refer to the +1/+2 position at the 5′ end of intron and a 3′SS can refer to the last two positions at the 3′ end of an intron. Alternatively, a 5′ SS can refer to the 5′ end of an exon and a 3′SS can refer to the 3′ end of an exon. In some embodiments, nucleic acids can contain one or more elements that act as a signal during transcription, splicing, and/or translation. In some embodiments, signaling elements include a 5′SS, a 3′SS, a premature stop codon, U1 and/or U2 binding sequences, and cis acting elements such as branch site (BS), polypyridine tract (PYT), exonic and intronic splicing enhancers (ESEs and ISEs) or silencers (ESSs and ISSs). In some embodiments, nucleic acids also comprise an untranslated region (UTR), such as a 5′ UTR or a 3′ UTR. In some embodiments, the start of an exon or intron is referred to interchangeably herein as the 5′ end of an exon or intron, respectively. Likewise, in some embodiments, the end of an exon or intron is referred to interchangeably herein as the 3′ end of an exon or intron, respectively.

In some embodiments, at least a portion of at least one target sequence is within about 1, about 5 or more, about 10 or more, about 15 or more, about 20 or more, about 25 or more, about 30 or more, about 35 or more, about 40 or more, about 45 or more, about 50 or more, about 55 or more, about 60 or more, about 65 or more, about 70 or more, about 75 or more, about 80 or more, about 85 or more, about 90 or more, about 95 or more, about 100 or more, about 105 or more, about 110 or more, about 115 or more, about 120 or more, about 125 or more, about 130 or more, about 135 or more, about 140 or more, about 145 or more, or about 150 to about 300 nucleotides adjacent to: the 5′ end of an exon; the 3′ end of an exon; the 5′ end of an intron; the 3′ end of an intron; one or more signaling element comprising a 5′SS, a 3′SS, a premature stop codon, U1 binding sequence, U2 binding sequence, a BS, a PYT, ESE, an ISE, an ESS, an ISS; a 5′ UTR; a 3′ UTR; more than one of the foregoing, or any combination thereof. In some embodiments, the target nucleic acid comprises a target locus. In some embodiments, the target nucleic acid comprises more than one target loci. In some embodiments, the target nucleic acid comprises two target loci. Accordingly, in some embodiments, the target nucleic acid can comprise one or more target sequences.

In some embodiments, compositions, systems, and methods described herein comprise a modified target nucleic acid which can describe a target nucleic acid wherein the target nucleic acid has undergone a change, for example, after contact with a polypeptide (e.g., effector protein, effector partner, fusion partner, fusion protein, or combinations thereof). In some embodiments, the modifying is an alteration in the nucleotide sequence of the target nucleic acid. In some embodiments, the edited target nucleic acid comprises a nicked target strand or a nicked non-target strand. In some embodiments, the modified target nucleic acid comprises an insertion, deletion, or replacement of one or more nucleotides compared to the unmodified target nucleic acid. In some embodiments, the modifying is a mutation.

### Mutations

In some embodiments, target nucleic acids described herein comprise a mutation. In some embodiments, a composition, system or method described herein can be used to modify a target nucleic acid comprising a mutation such that the mutation is modified to be the wild-type nucleotide or nucleotide sequence. In some embodiments, a composition, system or method described herein can be used to detect a target nucleic acid comprising a mutation. In some embodiments, a mutation results in the insertion of at least one amino acid in a protein encoded by the target nucleic acid. In some embodiments, a mutation results in the deletion of at least one amino acid in a protein encoded by the target nucleic acid. In some embodiments, a mutation results in the substitution of at least one amino acid in a protein encoded by the target nucleic acid. A mutation that results in the deletion, insertion, or substitution of one or more amino acids of a protein encoded by the target nucleic acid results in misfolding of a protein encoded by the target nucleic acid. In some embodiments, a mutation results in a premature stop codon, thereby resulting in a truncation of the encoded protein.

Non-limiting examples of mutations are insertion-deletion (indel), a point mutation, single nucleotide polymorphism (SNP), a chromosomal mutation, a copy number mutation or variation, and frameshift mutations. In some embodiments, an indel mutation is an insertion or deletion of one or more nucleotides. In some embodiments, a point mutation comprises a substitution, insertion, or deletion. In some embodiments, a frameshift mutation occurs when the number of nucleotides in the insertion/deletion is not divisible by three, and it occurs in a protein coding region. In some embodiments, a chromosomal mutation can comprise an inversion, a deletion, a duplication, or a translocation of one or more nucleotides. In some embodiments, a copy number variation can comprise a gene amplification or an expanding trinucleotide repeat. In some embodiments, an SNP is associated with a phenotype of the sample or a phenotype of the organism from which the sample was taken. In some embodiments, an SNP is associated with altered phenotype from wild type phenotype. In some embodiments, the SNP is a synonymous substitution or a nonsynonymous substitution. In some embodiments, the nonsynonymous substitution is a missense substitution or a nonsense point mutation. In some embodiments, the synonymous substitution is a silent substitution.

In some embodiments, a target nucleic acid described herein comprises a mutation of one or more nucleotides. In some embodiments, the one or more nucleotides comprises 1, 2, 3, 4, 5, 6, 7, 8, 9, 10, 11, 12, 13, 14, 15, 16, 17, 18, 19, 20 or more nucleotides. In some embodiments, the mutation comprises a deletion, insertion, and/or substitution of about 5, about 10, about 15, about 20, about 25, about 30, about 35, about 40, about 45, about 50, about 55, about 60, about 65, about 70, about 75, about 80, about 85, about 90, about 95, about 100, about 200, about 300, about 400, about 500, about 600, about 700, about 800, about 900, or about 1000 nucleotides. In some embodiments, the mutation comprises a deletion, insertion, and/or substitution of 1 to 5, 5 to 10, 10 to 15, 15 to 20, 20 to 25, 25 to 30, 30 to 35, 35 to 40, 40 to 45, 45 to 50, 50 to 55, 55 to 60, 60 to 65, 65 to 70, 70 to 75, 75 to 80, 80 to 85, 85 to 90, 90 to 95, 95 to 100, 100 to 200, 200 to 300, 300 to 400, 400 to 500, 500 to 600, 600 to 700, 700 to 800, 800 to 900, 900 to 1000, 1 to 50, 1 to 100, 25 to 50, 25 to 100, 50 to 100, 100 to 500, 100 to 1000, or 500 to 1000 nucleotides. In some embodiments, the mutation is located in a non-coding region or a coding region of a gene, wherein the gene is a target nucleic acid. In some embodiments, a mutation is in an open reading frame of a target nucleic acid. In some embodiments, guide nucleic acids described herein hybridize to a portion of the target nucleic acid comprising or adjacent to the mutation.

In some embodiments, the target nucleic acid comprises one or more mutations. In some embodiments, the target nucleic acid comprises one or more, two or more, three or more, four or more, five or more, six or more, seven or more, eight or more, nine or more, or ten or more mutations as compared to the unmutated target nucleic acid. In some embodiments, the target nucleic acid comprises a sequence comprising one or more, two or more, three or more, four or more, five or more, six or more, seven or more, eight or more, nine or more, or ten or more mutations as compared to the wildtype sequence. In some embodiments, the target nucleic acid comprises a mutation associated with a disease or disorder.

In some embodiments, target nucleic acids comprise a mutation, wherein the mutation is a SNP. In some embodiments, the single nucleotide mutation or SNP is associated with a phenotype of the sample or a phenotype of the organism from which the sample was taken. In some embodiments, the SNP is associated with altered phenotype from wild type phenotype. In some embodiments, a single nucleotide mutation, SNP, or deletion described herein is associated with a disease, such as a genetic disease. In some embodiments, the SNP is a synonymous substitution or a nonsynonymous substitution. In some embodiments, the nonsynonymous substitution is a missense substitution or a nonsense point mutation. In some embodiments, the synonymous substitution is a silent substitution. In some embodiments, the mutation is a deletion of one or more nucleotides. In some embodiments, the single nucleotide mutation, SNP, or deletion is associated with a disease such as a genetic disorder. In some embodiments, the mutation, such as a single nucleotide mutation, a SNP, or a deletion, is encoded in the nucleotide sequence of a target nucleic acid from the germline of an organism or is encoded in a target nucleic acid from a diseased cell.

In some embodiments, the mutation is associated with a disease, such as a genetic disorder. In some embodiments, the mutation is encoded in the nucleotide sequence of a target nucleic acid from the germline of an organism or is encoded in a target nucleic acid from a diseased cell. In some embodiments, a target nucleic acid described herein comprises a mutation associated with a disease. In some examples, a mutation associated with a disease refers to a mutation whose presence in a subject indicates that the subject is susceptible to or suffers from, a disease, disorder, condition, or syndrome. In some examples, a mutation associated with a disease refers to a mutation which causes, contributes to the development of, or indicates the existence of the disease, disorder, condition, or syndrome. In some embodiments, a mutation associated with a disease also refers to any mutation which generates transcription or translation products at an abnormal level, or in an abnormal form, in cells affected by a disease relative to a control without the disease. In some examples, a mutation associated with a disease refers to a mutation whose presence in a subject indicates that the subject is susceptible to, or suffers from, a disease, disorder, or pathological state. In some embodiments, a mutation associated with a disease, comprises the co-occurrence of a mutation and the phenotype of a disease. In some embodiments, the mutation occurs in a gene, wherein transcription or translation products from the gene occur at a significantly abnormal level or in an abnormal form in a cell or subject harboring the mutation as compared to a non-disease control subject not having the mutation. In some embodiments, a target nucleic acid described herein comprises a mutation associated with a disease, wherein the target nucleic acid is any one of the target nucleic acids set forth in TABLE 4 and TABLE 4.1. In some embodiments, a target nucleic acid described herein comprises a mutation associated with a disease, wherein the disease is any one of the diseases set forth in TABLE 5 and TABLE 5.1.

### Detection of Target Nucleic Acids

Described herein are devices, systems, fluidic devices, kits, and methods for detecting the presence of a target nucleic acid in a sample. In some embodiments, a target nucleic acid described herein comprises a mutation associated with a disease, wherein the target nucleic acid is any one of the target nucleic acids set forth in TABLE 4 and TABLE 4.1. In some embodiments, a target nucleic acid described herein comprises a mutation associated with a disease, wherein the disease is any one of the diseases set forth in TABLE 5 and TABLE 5.1. In some embodiments, a target nucleic acid is in a cell. In some embodiments, the cell is a single-cell eukaryotic organism; a plant cell an algal cell; a fungal cell; an animal cell; a cell of an invertebrate animal; a cell of a vertebrate animal such as fish, amphibian, reptile, bird, and mammal; or a cell of a mammal such as a human, a non-human primate, an ungulate, a feline, a bovine, an ovine, and a caprine. In some embodiments, the cell is a eukaryotic cell. In some embodiments, the cell is a mammalian cell, a human cell, or a plant cell. In some embodiments, the cell is a human cell. In some embodiments, the human cell is a: muscle cell, liver cell, lung cell, cardiac cell, visceral cell, cardiac muscle cell, smooth muscle cell, cardiomyocyte, nodal cardiac muscle cell, smooth muscle cell, visceral muscle cell, skeletal muscle cell, myocyte, red (or slow) skeletal muscle cell, white (fast) skeletal muscle cell, intermediate skeletal muscle, muscle satellite cell, muscle stem cell, myoblast, muscle progenitor cell, induced pluripotent stem cell (iPS), or a cell derived from an iPS cell, modified to have its gene edited and differentiated into myoblasts, muscle progenitor cells, muscle satellite cells, muscle stem cells, skeletal muscle cells, cardiac muscle cells or smooth muscle cells.

In some embodiments, an effector protein-guide nucleic acid complex comprises high selectivity for a target sequence. In some embodiments, an RNP comprise a selectivity of at least 200:1, 100:1, 50:1, 20:1, 10:1, or 5:1 for a target nucleic acid over a single nucleotide variant of the target nucleic acid. In some embodiments, an RNP comprises a selectivity of at least 5:1 for a target nucleic acid over a single nucleotide variant of the target nucleic acid.

By leveraging such effector protein selectivity, some methods described herein detects a target nucleic acid present in the sample in various concentrations or amounts as a target nucleic acid population. In some embodiments, the method detects at least 2 target nucleic acid populations. In some embodiments, the method detects at least 3, 4, 5, 6, 7, 8, 9, 10, 20, 30, 40, or 50 target nucleic acid populations. In some embodiments, the method detects 3 to 50, 5 to 40, or 10 to 25 target nucleic acid populations. In some embodiments, the method detects at least 2 individual target nucleic acids. In some embodiments, the method detects at least 3, 5, 10, 20, 30, 40, 50, 100, 200, 300, 400, 500, 600, 700, 800, 900, 1000, 2000, 3000, 4000, 5000, 6000, 7000, 8000, 9000, or 10000 individual target nucleic acids. In some embodiments, the method detects 1 to 10,000, 100 to 8000, 400 to 6000, 500 to 5000, 1000 to 4000, or 2000 to 3000 individual target nucleic acids. In some embodiments, the method detects target nucleic acid present at least at one copy per 10 non-target nucleic acids, 10² non-target nucleic acids, 10³ non-target nucleic acids, 10⁴ non-target nucleic acids, 10⁵ non-target nucleic acids, 10⁶ non-target nucleic acids, 10⁷ non-target nucleic acids, 10⁸ non-target nucleic acids, 10⁹ non-target nucleic acids, or 10¹⁰ non-target nucleic acids.

In some embodiments, compositions described herein exhibit indiscriminate trans cleavage of a nucleic acid (e.g., a ssRNA), enabling their use for detection of a nucleic acid (e.g., RNA) in samples. In some embodiments, target nucleic acids are generated from many nucleic acid templates (e.g., RNA) in order to achieve cleavage of a reporter (e.g., a FQ reporter) in a device (e.g., a DETECTR platform). In some embodiments, certain effector proteins are activated by a nucleic acid (e.g., a ssRNA), upon which they exhibit trans cleavage of the nucleic acid (e.g., ssRNA) and are, thereby, used for cleaving the reporter molecules (e.g., ssRNA FQ reporter molecules) in a device (e.g., a DETECTR system). In some embodiments, the effector proteins target nucleic acids present in the sample or nucleic acids generated and/or amplified from any number of nucleic acid templates (e.g., RNA). Described herein are reagents comprising a single stranded reporter nucleic acid comprising a detection moiety, wherein the reporter nucleic acid (e.g., a ssDNA-FQ reporter described herein) is cleaved by the effector protein, upon generation and/or amplification of nucleic acids from a nucleic acid template (e.g., ssRNA) using the methods disclosed herein, thereby generating a first detectable signal. While DNA is used as an exemplary reporter in the foregoing, any suitable reporter may be used. Suitable reporters may comprise DNA and RNA.

In some embodiments, a target nucleic acid is an amplified nucleic acid of interest. In some embodiments, the nucleic acid of interest is any nucleic acid disclosed herein or from any sample as disclosed herein. In some embodiments, the nucleic acid of interest is DNA. In some embodiments, the nucleic acid of interest is an RNA. In some embodiments, the nucleic acid of interest is an RNA that is reverse transcribed before amplification. In some embodiments, the target nucleic acid is associated to an amplicon (DNA) or is an amplicon of a target nucleic acid (DNA or RNA) generated via amplification (with or without reverse transcription). In some embodiments, the target nucleic acid is an amplicon of a target nucleic acid (DNA or RNA) generated via amplification that is reverse transcribed before amplification.

In some embodiments, target nucleic acids activate an effector protein to initiate sequence-independent cleavage of a nucleic acid-based reporter (e.g., a reporter comprising an RNA sequence, or a reporter comprising DNA and RNA). For example, an effector protein of the present disclosure is activated by a target nucleic acid to cleave reporters having an RNA (also referred to herein as an “RNA reporter”). Alternatively, an effector protein of the present disclosure is activated by a target nucleic acid to cleave reporters having an RNA. Alternatively, an effector protein of the present disclosure is activated by a target RNA to cleave reporters having an RNA (also referred to herein as a “RNA reporter”). In some embodiments, the RNA reporter comprises a single-stranded RNA labelled with a detection moiety or any RNA reporter as disclosed herein.

In some embodiments, systems described herein comprise a reporter as described herein. In some embodiments, the reporter is cleaved by the polypeptide (e.g., effector protein, effector partner, fusion partner, fusion protein, or combinations thereof). In some embodiments, the reporter is cleaved by the polypeptide (e.g., effector protein, effector partner, fusion partner, fusion protein, or combinations thereof) or the reporter is configured to release a detection moiety when cleaved by the polypeptide (e.g., effector protein, effector partner, fusion partner, fusion protein, or combinations thereof) following hybridizing of an engineered guide nucleic acid to the target nucleic acid, and wherein release of the detection moiety is indicative of a presence or absence of the target nucleic acid.

Further description of editing or detecting a target nucleic acid in a gene of interest can be found in more detail in Kim et al., “Enhancement of target specificity of CRISPR-Cas12a by using a chimeric DNA-RNA guide”, Nucleic Acids Res. 2020 Sep. 4; 48(15):8601-8616; Wang et al., “Specificity profiling of CRISPR system reveals greatly enhanced off-target gene editing”, Scientific Reports volume 10, Article number: 2269 (2020); Tuladhar et al., “CRISPR-Cas9-based mutagenesis frequently provokes on-target mRNA misregulation”, Nature Communications volume 10, Article number: 4056 (2019); Dong et al., “Genome-Wide Off-Target Analysis in CRISPR-Cas9 Modified Mice and Their Offspring”, G3, Volume 9, Issue 11, 1 Nov. 2019, Pages 3645-3651; Winter et al., “Genome-wide CRISPR screen reveals novel host factors required for Staphylococcus aureus α-hemolysin-mediated toxicity”, Scientific Reports volume 6, Article number: 24242 (2016); and Ma et al., “A CRISPR-Based Screen Identifies Genes Essential for West-Nile-Virus-Induced Cell Death”, Cell Rep. 2015 Jul. 28; 12(4):673-83, which are hereby incorporated by reference in their entirety.

### Samples

Various sample types comprising a target nucleic acid of interest are consistent with the present disclosure. In some embodiments, these samples comprise a target nucleic acid for detection. In some embodiments, the detection of the target nucleic indicates an ailment, such as a disease, cancer, or genetic disorder, or genetic information, such as for phenotyping, genotyping, or determining ancestry and are compatible with the reagents and support mediums as described herein. Generally, a sample from an individual or an animal or an environmental sample is obtained for testing presence of a disease, cancer, genetic disorder, or any mutation of interest.

In some embodiments, a sample comprises a target nucleic acid from 0.05% to 20% of total nucleic acids in the sample. In some embodiments, the target nucleic acid is 0.1% to 10% of the total nucleic acids in the sample. In some embodiments, the target nucleic acid is 0.1% to 5% of the total nucleic acids in the sample. In some embodiments, the target nucleic acid is 0.1% to 1% of the total nucleic acids in the sample. In some embodiments, the target nucleic acid is in any amount less than 100% of the total nucleic acids in the sample. In some embodiments, the target nucleic acid is 100% of the total nucleic acids in the sample. In some embodiments, the sample comprises a portion of the target nucleic acid and at least one nucleic acid comprising less than 100% sequence identity to the portion of the target nucleic acid but no less than 50% sequence identity to the portion of the target nucleic acid. For example, the portion of the target nucleic acid comprises a mutation as compared to at least one nucleic acid comprising less than 100% sequence identity to the portion of the target nucleic acid but no less than 50% sequence identity to the portion of the target nucleic acid. In some embodiments, the portion of the target nucleic acid comprises a single nucleotide mutation as compared to at least one nucleic acid comprising less than 100% sequence identity to the portion of the target nucleic acid but no less than 50% sequence identity to the portion of the target nucleic acid.

In some embodiments, a sample comprises target nucleic acid populations at different concentrations or amounts. In some embodiments, the sample has at least 2 target nucleic acid populations. In some embodiments, the sample has at least 3, 4, 5, 6, 7, 8, 9, 10, 20, 30, 40, or 50 target nucleic acid populations. In some embodiments, the sample has 3 to 50, 5 to 40, or 10 to 25 target nucleic acid populations.

In some embodiments, a sample has at least 2 individual target nucleic acids. In some embodiments, the sample has at least 3, 5, 10, 20, 30, 40, 50, 100, 200, 300, 400, 500, 600, 700, 800, 900, 1000, 2000, 3000, 4000, 5000, 6000, 7000, 8000, 9000, or 10000 individual target nucleic acids. In some embodiments, the sample comprises 1 to 10,000, 100 to 8000, 400 to 6000, 500 to 5000, 1000 to 4000, or 2000 to 3000 individual target nucleic acids.

In some embodiments, a sample comprises one copy of target nucleic acid per 10 non-target nucleic acids, 10² non-target nucleic acids, 10³ non-target nucleic acids, 10⁴ non-target nucleic acids, 10⁵ non-target nucleic acids, 10⁶ non-target nucleic acids, 10⁷ non-target nucleic acids, 10⁸ non-target nucleic acids, 10⁹ non-target nucleic acids, or 10¹⁰ non-target nucleic acids.

In some embodiments, samples comprise a target nucleic acid at a concentration of less than 1 nM, less than 2 nM, less than 3 nM, less than 4 nM, less than 5 nM, less than 6 nM, less than 7 nM, less than 8 nM, less than 9 nM, less than 10 nM, less than 20 nM, less than 30 nM, less than 40 nM, less than 50 nM, less than 60 nM, less than 70 nM, less than 80 nM, less than 90 nM, less than 100 nM, less than 200 nM, less than 300 nM, less than 400 nM, less than 500 nM, less than 600 nM, less than 700 nM, less than 800 nM, less than 900 nM, less than 1 μM, less than 2 μM, less than 3 μM, less than 4 μM, less than 5 μM, less than 6 μM, less than 7 μM, less than 8 μM, less than 9 μM, less than 10 μM, less than 100 μM, or less than 1 mM. In some embodiments, the sample comprises a target nucleic acid at a concentration of 1 nM to 2 nM, 2 nM to 3 nM, 3 nM to 4 nM, 4 nM to 5 nM, 5 nM to 6 nM, 6 nM to 7 nM, 7 nM to 8 nM, 8 nM to 9 nM, 9 nM to 10 nM, 10 nM to 20 nM, 20 nM to 30 nM, 30 nM to 40 nM, 40 nM to 50 nM, 50 nM to 60 nM, 60 nM to 70 nM, 70 nM to 80 nM, 80 nM to 90 nM, 90 nM to 100 nM, 100 nM to 200 nM, 200 nM to 300 nM, 300 nM to 400 nM, 400 nM to 500 nM, 500 nM to 600 nM, 600 nM to 700 nM, 700 nM to 800 nM, 800 nM to 900 nM, 900 nM to 1 μM, 1 μM to 2 μM, 2 μM to 3 μM, 3 μM to 4 μM, 4 μM to 5 μM, 5 μM to 6 μM, 6 μM to 7 μM, 7 μM to 8 μM, 8 μM to 9 μM, 9 μM to 10 μM, 10 μM to 100 μM, 100 μM to 1 mM, 1 nM to 10 nM, 1 nM to 100 nM, 1 nM to 1 μM, 1 nM to 10 μM, 1 nM to 100 μM, 1 nM to 1 mM, 10 nM to 100 nM, 10 nM to 1 μM, 10 nM to 10 μM, 10 nM to 100 μM, 10 nM to 1 mM, 100 nM to 1 μM, 100 nM to 10 μM, 100 nM to 100 μM, 100 nM to 1 mM, 1 μM to 10 μM, 1 μM to 100 μM, 1 μM to 1 mM, 10 μM to 100 μM, 10 μM to 1 mM, or 100 μM to 1 mM. In some embodiments, the sample comprises a target nucleic acid at a concentration of 20 nM to 200 μM, 50 nM to 100 μM, 200 nM to 50 μM, 500 nM to 20 μM, or 2 μM to 10 μM. In some embodiments, the target nucleic acid is not present in the sample.

In some embodiments, samples comprise fewer than 10 copies, fewer than 100 copies, fewer than 1,000 copies, fewer than 10,000 copies, fewer than 100,000 copies, or fewer than 1,000,000 copies of a target nucleic acid. In some embodiments, the sample comprises 10 copies to 100 copies, 100 copies to 1,000 copies, 1,000 copies to 10,000 copies, 10,000 copies to 100,000 copies, 100,000 copies to 1,000,000 copies, 10 copies to 1,000 copies, 10 copies to 10,000 copies, 10 copies to 100,000 copies, 10 copies to 1,000,000 copies, 100 copies to 10,000 copies, 100 copies to 100,000 copies, 100 copies to 1,000,000 copies, 1,000 copies to 100,000 copies, or 1,000 copies to 1,000,000 copies of a target nucleic acid. In some embodiments, the sample comprises 10 copies to 500,000 copies, 200 copies to 200,000 copies, 500 copies to 100,000 copies, 1,000 copies to 50,000 copies, 2,000 copies to 20,000 copies, 3,000 copies to 10,000 copies, or 4,000 copies to 8,000 copies. In some embodiments, the target nucleic acid is not present in the sample.

In some embodiments, the sample is a biological sample, an environmental sample, or a combination thereof. Non-limiting examples of biological samples are blood, serum, plasma, saliva, urine, mucosal sample, peritoneal sample, cerebrospinal fluid, gastric secretions, nasal secretions, sputum, pharyngeal exudates, urethral or vaginal secretions, an exudate, an effusion, and a tissue sample (e.g., a biopsy sample). In some embodiments, a tissue sample from a subject is dissociated or liquified prior to application to detection system of the present disclosure. Non-limiting examples of environmental samples are soil, air, or water. In some embodiments, an environmental sample is taken as a swab from a surface of interest or taken directly from the surface of interest.

In some embodiments, the sample is a raw (unprocessed, unedited, unmodified) sample. In some embodiments, raw samples are applied to a system for detecting or modifying a target nucleic acid, such as those described herein. In some embodiments, the sample is diluted with a buffer or a fluid or concentrated prior to its application to the system or be applied neat to the detection system. Sometimes, the sample contains no more 20 μl of buffer or fluid. The sample, in some embodiments, is contained in no more than 0.01, 0.1, 0.5, 1, 5, 10, 15, 20, 25, 30, 35 40, 45, 50, 55, 60, 65, 70, 75, 80, 90, 100, 200, 300, 400, 500 μl, or any of value 0.01 μl to 500 μl, 0.1 μL to 100 μL, or more preferably 1 μL to 50 μL of buffer or fluid. Sometimes, the sample is contained in more than 500 μl. In some embodiments, the compositions, systems, devices, kits, and methods disclosed herein are compatible with the buffers or fluid disclosed herein.

In some embodiments, the sample is taken from a single-cell eukaryotic organism; a plant or a plant cell; an algal cell; a fungal cell; an animal cell, tissue, or organ; a cell, tissue, or organ from an invertebrate animal; a cell, tissue, fluid, or organ from a vertebrate animal such as fish, amphibian, reptile, bird, and mammal; a cell, tissue, fluid, or organ from a mammal such as a human, a non-human primate, an ungulate, a feline, a bovine, an ovine, and a caprine. In some embodiments, the sample is taken from nematodes, protozoans, helminths, or malarial parasites. In some embodiments, the sample comprises nucleic acids from a cell lysate from a eukaryotic cell, a mammalian cell, a human cell, a prokaryotic cell, or a plant cell. In some embodiments, the sample comprises nucleic acids expressed from a cell.

In some embodiments, samples are used for diagnosing a disease. In some embodiments the disease is cancer. In some embodiments, the sample used for cancer testing comprises at least one target nucleic acid that hybridizes to a guide nucleic acid of the reagents described herein. The target nucleic acid, in some embodiments, comprises a nucleic acid associated with a portion of a gene comprising a mutation associated with a disease, such as cancer, a gene whose overexpression is associated with cancer, a tumor suppressor gene, an oncogene, a checkpoint inhibitor gene, a gene associated with cellular growth, a gene associated with cellular metabolism, or a gene associated with cell cycle. Sometimes, the target nucleic acid encodes a cancer biomarker or is associated with a cancer biomarker. In some embodiments, the assay is used to detect “hotspots” in target nucleic acids that are predictive of a cancer. In some embodiments, the target nucleic acid comprises a portion of a nucleic acid that is associated with a cancer.

In some embodiments, the target nucleic acid is a portion of a nucleic acid associated with a genomic locus, a nucleic acid associated with any DNA amplicon or a cDNA, a circular RNA, a reverse transcribed mRNA, or any RNA associated with a locus of at least one gene set forth in TABLE 4 and TABLE 4.1. Any region of the aforementioned gene loci is probed for a mutation or deletion using the compositions and methods disclosed herein. For example, in the EGFR gene locus, the compositions and methods for detection disclosed herein are used for detecting a single nucleotide polymorphism or a deletion.

In some embodiments, samples are used to diagnose a genetic disorder, also referred to as genetic disorder testing. In some embodiments, the sample used for genetic disorder testing comprises at least one target nucleic acid that hybridizes to a guide nucleic acid of the reagents described herein. The target nucleic acid, in some embodiments, is associated with a gene comprising a mutation associated with a genetic disorder, associated with a gene whose overexpression is associated with a genetic disorder, associated with a gene associated with abnormal cellular growth resulting in a genetic disorder, or associated with a gene associated with abnormal cellular metabolism resulting in a genetic disorder. In some embodiments, the target nucleic acid is a nucleic acid associated with a genomic locus, a nucleic acid associated with a DNA amplicon or cDNA, a mRNA, or a reverse transcribed mRNA, a circular RNA, or any RNA associated with a locus of at least one gene set forth in TABLE 4 and TABLE 4.1.

In some embodiments, a sample used for phenotyping testing comprises at least one target nucleic acid that hybridizes to a guide nucleic acid of the reagents described herein. The target nucleic acid, in some embodiments, is a nucleic acid encoding a sequence associated with a phenotypic trait. In some embodiments, a sample used for genotyping testing comprises at least one target nucleic acid that hybridizes to a guide nucleic acid of the reagents described herein. A target nucleic acid, in some embodiments, is a nucleic acid associated with a genotype of interest. In some embodiments, a sample used for ancestral testing comprises at least one target nucleic acid that hybridizes to a guide nucleic acid of the reagents described herein. A target nucleic acid, in some embodiments, is a nucleic acid encoding a sequence associated with a geographic region of origin or ethnic group. In some embodiments, a sample is used for identifying a disease status. For example, a sample is any sample described herein, and is obtained from a subject for use in identifying a disease status of a subject. In some embodiments, the disease is cancer. In some embodiments, the disease is a genetic disorder. In some embodiments, a method comprises obtaining a serum sample from a subject; and identifying a disease status of the subject.

### IX. Compositions

Disclosed herein are compositions comprising one or more polypeptides (e.g., effector proteins, effector partners, fusion partners, fusion proteins, or combinations thereof) described herein or nucleic acids encoding the one or more polypeptides, one or more guide nucleic acids described herein or nucleic acids encoding the one or more guide nucleic acids described herein, or combinations thereof. In some embodiments, one or more of a repeat sequence of the one or more guide nucleic acids interact with the one or more of the effector proteins. In some embodiments, spacer sequences of the one or more guide nucleic acids hybridizes with a target sequence of a target nucleic acid. In some embodiments, the compositions cleave a target strand, a non-target strand, or both. In some embodiments, the compositions do not cleave a target strand, a non-target strand, or both. In some embodiments, the compositions modify a target strand or a non-target strand. In some embodiments, the compositions modify expression of the target nucleic acids, proteins associated with the expression of the target nucleic acids, other nucleic acids associated with the target nucleic acids, or combinations thereof. In some embodiments, the compositions modify a target nucleic acid in a cell or a subject. In some embodiments, the compositions modify a target nucleic acid or the expression thereof in a cell, in a tissue, in an organ, in vitro, in vivo, or ex vivo. In some embodiments, the compositions modify a target nucleic acid in a sample comprising the target nucleic.

In some embodiments, compositions described herein comprise plasmids described herein, viral vectors described herein, non-viral vectors described herein, or combinations thereof. In some embodiments, compositions described herein comprise the viral vectors. In some embodiments, compositions described herein comprise an AAV. In some embodiments, compositions described herein comprise liposomes (e.g., cationic lipids or neutral lipids), dendrimers, lipid nanoparticle (LNP), or cell-penetrating peptides. In some embodiments, compositions described herein comprise an LNP.

Also described herein are compositions comprising: (a) a polypeptide, or a nucleic acid encoding the polypeptide, and an engineered guide nucleic acid, or a nucleic acid that encodes the engineered guide nucleic acid; (b) a polypeptide, or a nucleic acid encoding the polypeptide, and an engineered guide nucleic acid comprising a crRNA; (c) an mRNA encoding a polypeptide, and an engineered guide nucleic acid; (d) one or more recombinant expression vectors comprising: i) a nucleic acid encoding a polypeptide; and ii) an engineered guide nucleic acid; or (e) one or more recombinant expression vectors comprising: i) a nucleic acid encoding a polypeptide; and ii) an engineered guide nucleic acid comprising a crRNA; wherein the polypeptide comprises an amino acid sequence that is at least 85% identical to any one of the sequences set forth in TABLE 1 or TABLE 1.2.

### Pharmaceutical Compositions

Described herein are formulations of introducing compositions or components of a system described herein to a host.

In some embodiments, compositions described herein are pharmaceutical compositions. In some embodiments, the pharmaceutical compositions comprise compositions described herein and a pharmaceutically acceptable carrier or diluent. In some embodiments, the pharmaceutical compositions comprise compositions described herein or systems described herein. In some embodiments, the pharmaceutical composition comprises a pharmaceutically acceptable salt, one or more of a vehicle, adjuvant, excipient, or carrier, such as a filler, disintegrant, a surfactant, a binder, a lubricant, or combinations thereof. Remington: The Science and Practice of Pharmacy, 21st edition, 2005, ed. D. B. Troy, Lippincott Williams & Wilkins, Philadelphia; Encyclopedia of Pharmaceutical Technology, eds. J. Swarbrick and J. C. Boylan, 1988-1999, Marcel Dekker, New York; and Encyclopedia of Pharmaceutical Technology, eds. J. Swarbrick, 2015, CRC Press, Boca Raton disclose various carriers used in formulating pharmaceutically acceptably compositions and known techniques for the preparation thereof. Non-limiting examples of pharmaceutically acceptable carriers and diluents suitable for the pharmaceutical compositions disclosed herein include buffers (e.g., neutral buffered saline, phosphate buffered saline); carbohydrates (e.g., glucose, mannose, sucrose, dextran, mannitol); polypeptides or amino acids (e.g., glycine); antioxidants; chelating agents (e.g., EDTA, glutathione); adjuvants (e.g., aluminum hydroxide); surfactants (Polysorbate 80, Polysorbate 20, or Pluronic F68); glycerol; sorbitol; mannitol; polyethyleneglycol; and preservatives. In some embodiments, the vector is formulated for delivery through injection by a needle carrying syringe. In some embodiments, the composition is formulated for delivery by electroporation. In some embodiments, the composition is formulated for delivery by chemical method. In some embodiments, the pharmaceutical compositions comprise a virus vector or a non-viral vector.

Pharmaceutical compositions described herein comprise a salt. In some embodiments, the salt is a sodium salt. In some embodiments, the salt is a potassium salt. In some embodiments, the salt is a magnesium salt. In some embodiments, the salt is NaCl. In some embodiments, the salt is KNO₃. In some embodiments, the salt is Mg²⁺ SO₄²⁻.

Pharmaceutical compositions described herein are in the form of a solution (e.g., a liquid). In some embodiments, the solution is formulated for injection, e.g., intravenous or subcutaneous injection. In some embodiments, the pH of the solution is about 7, about 7.1, about 7.2, about 7.3, about 7.4, about 7.5, about 7.6, about 7.7, about 7.8, about 7.9, about 8, about 8.1, about 8.2, about 8.3, about 8.4, about 8.5, about 8.6, about 8.7, about 8.8, about 8.9, or about 9. In some embodiments, the pH is 7 to 7.5, 7.5 to 8, 8 to 8.5, 8.5 to 9, or 7 to 8.5. In some cases, the pH of the solution is less than 7. In some cases, the pH is greater than 7.

### X. Systems

Disclosed herein, in some aspects, are systems for detecting a target nucleic acid, comprising any one of the effector proteins described herein. In some embodiments, systems comprise a guide nucleic acid described herein. In some embodiments, systems comprise one or more components having a guide nucleic acid. In some embodiments, systems comprise one or more components having a guide nucleic acid and an additional nucleic acid. In some embodiments, systems are used for detecting a target nucleic acid. In some embodiments, systems are used for modifying or editing a target nucleic acid. In some embodiments, systems comprise an effector protein described herein, one or more guide nucleic acids, a reagent, a support medium, or combinations thereof. In some embodiments, systems comprise compositions, a solution, a buffer, a reagent, a support medium, or combinations thereof. In some embodiments, systems further comprise a donor nucleic acid as disclosed herein. In some embodiments, systems or system components described herein are comprised in a single composition.

In some embodiments, systems described herein are systems for detecting of a target nucleic acid. In some embodiments, a system for detecting a target nucleic acid comprises any one of the systems described herein, and a reporter, wherein the reporter comprises a nucleic acid and a detectable moiety, and wherein the nucleic acid comprises RNA, ssDNA, or a combination thereof. In some embodiments, cleavage of the reporter generates a detectable product or detectable signal from the detectable moiety. In some embodiments, cleavage of the reporter reduces a detectable signal from the detectable moiety. In some embodiments, cleavage of the reporter is effective to produce a detectable product comprising a detectable moiety. In some embodiments, the detectable moiety comprises a fluorophore, a quencher, a FRET (fluorescence resonance energy transfer) pair, a fluorescent protein, horseradish peroxidase protein, a colorimetric signal, an antigen or a combination thereof. In some embodiments, the reporter comprises a fluorophore which is attached to a quencher by the nucleic acid, and wherein, upon cleavage of the nucleic acid, the fluorophore generates a signal, wherein the signal is detected as a positive signal, indicating the presence of the target nucleic acid. In some embodiments, the reporter is configured to generate a signal indicative of a presence or absence of the target nucleic acid. In some embodiments, the reporter is cleaved by the polypeptide. In some embodiments, the reporter is configured to release a detection moiety when cleaved by the polypeptide following hybridizing of the engineered guide nucleic acid to the target nucleic acid, and wherein release of the detection moiety is indicative of a presence or absence of the target nucleic acid. In some embodiments, the reporter is operably linked to a polypeptide. In some embodiments, the target nucleic acid is isolated from a human cell.

In some embodiments, the system described herein further comprising at least one detection reagent for detecting a target nucleic acid. In some embodiments, the at least one detection reagent is selected from a reporter nucleic acid, a detection moiety, an additional polypeptide, or a combination thereof, optionally wherein the reporter nucleic acid comprises a fluorophore, a quencher, or a combination thereof. In some embodiments, the at least one detection reagent is operably linked to a polypeptide, such that a detection event occurs upon contacting the system with a target nucleic acid.

In some embodiments, the system comprising an engineered guide nucleic acid wherein the engineered guide nucleic acid is capable of hybridizing to a target sequence in a target nucleic acid, wherein the target nucleic acid is any one of: a naturally occurring eukaryotic sequence, an engineered eukaryotic sequence, a fragment of a naturally occurring eukaryotic sequence, a fragment of an engineered eukaryotic sequence, and combinations thereof.

Also provided herein are systems comprising a recombinant nucleic acid encoding a polypeptide, wherein the recombinant nucleic acid encoding the polypeptide is a nucleic acid expression vector, and optionally wherein the nucleic acid expression vector is a viral vector or an adeno associated viral (AAV) vector. In some embodiments, the nucleic acid expression vector is a viral vector. In some embodiments, the nucleic acid expression vector is an AAV vector. In some embodiments, the nucleic acid expression vector encodes at least one guide nucleic acid.

Also provided herein are system comprising an engineered polypeptide, or a recombinant nucleic acid encoding the engineered polypeptide, wherein the engineered polypeptide comprises an amino acid sequence that is at least 85% identical to any one of the sequences set forth in TABLE 1 or TABLE 1.2.

In some embodiments, any one of the systems described herein, any one of the kits described herein, any one of the devices described herein, or any one of the microfluidic devices described herein, wherein components of the system, kit, device, or microfluidic device are used in diagnosis of a disease or disorder.

In some embodiments, anyone of the systems described herein, anyone of the kits described herein, any one of the devices described herein, or any one of the microfluidic devices described herein, wherein components of the system, kit, device, or microfluidic device are used in diagnosis of a disease or disorder associated with a nucleic acid sequence modification in a disease or disorder associated gene selected from a viral genome, a prokaryotic genome, or an eukaryotic genome.

In some embodiments, any one of the systems described herein, any one of the kits described herein, any one of the devices described herein, or any one of the microfluidic devices described herein, wherein components of the system, kit, device, or microfluidic device are used in diagnosis of a disease or disorder associated with a non-wild type gene, a gene comprising a non-wild type reading frame; a gene comprising one or more mutations, or abnormal processing upon transcription of a gene.

In some embodiments, systems comprise a fusion protein described herein. In some embodiments, effector proteins comprise an amino acid sequence that is at least 70%, at least 75%, at least 80%, at least 85%, at least 90%, at least 95%, at least 98%, or at least 100% identical to any one of the amino acid sequences selected from TABLE 1 or TABLE 1.2. In some embodiments, the amino acid sequence of the effector protein is at least 70%, at least 75%, at least 80%, at least 85%, at least 90%, at least 95%, at least 98%, or at least 100% identical to any one of the amino acid sequences selected from TABLE 1 or TABLE 1.2.

In some embodiments, systems, compositions, methods, kits, devices, and solutions comprise 0.01 μL, 0.02 μL, 0.03 μL, 0.04 μL, 0.05 μL, 0.06 μL, 0.07 μL, 0.08 μL, 0.09 μL, 0.1 μL, 0.2 μL, 0.3 μL, 0.4 μL, 0.5 μL, 0.6 μL, 0.7 μL, 0.8 μL, 0.9 μL, 1 μL, 2 μL, 3 μL, 4 μL, 5 μL, 6 μL, 7 μL, 8 μL, 9 μL, 10 μL, 20 μL, 30 μL, 40 μL, 50 μL, 60 μL, 70 μL, 80 μL, 90 μL, 100 μL, 150 μL, 200 μL, 250 μL, 300 μL, 350 μL, 400 μL, 450 μL, 500 μL, or more, effector proteins, or nucleic acids encoding the effector proteins, as described herein. In some embodiments, systems, compositions, methods, kits, devices, and solutions comprise 1 nM, 2 nM, 3 nM, 4 nM, 5 nM, 6 nM, 7 nM, 8 nM, 9 nM, 10 nM, 20 nM, 30 nM, 40 nM, 50 nM, 60 nM, 70 nM, 80 nM, 90 nM, 100 nM, 150 nM, 200 nM, 250 nM, 300 nM, 350 nM, 400 nM, 450 nM, 500 nM, or more, effector proteins, or nucleic acids encoding the effector proteins, as described herein. In some embodiments, systems, compositions, methods, kits, devices, and solutions comprise 1 μM, 2 μM, 3 μM, 4 μM, 5 μM, 6 μM, 7 μM, 8 μM, 9 μM, 10 μM, 20 μM, 30 μM, 40 μM, 50 μM, 60 μM, 70 μM, 80 μM, 90 μM, 100 μM, 150 μM, 200 μM, 250 μM, 300 μM, 350 μM, 400 μM, 450 μM, 500 μM, or more, effector proteins, or nucleic acids encoding the effector proteins, as described herein. In some embodiments, systems, compositions, methods, kits, devices, and solutions comprise 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 150 mM, 200 mM, 250 mM, 300 mM, 350 mM, 400 mM, 450 mM, 500 mM, or more, effector proteins, or nucleic acids encoding the effector proteins, as described herein.

In some embodiments, systems, compositions, methods, kits, devices, and solutions comprise 0.01 μL, 0.02 μL, 0.03 μL, 0.04 μL, 0.05 μL, 0.06 μL, 0.07 μL, 0.08 μL, 0.09 μL, 0.1 μL, 0.2 μL, 0.3 μL, 0.4 μL, 0.5 μL, 0.6 μL, 0.7 μL, 0.8 μL, 0.9 μL, 1 μL, 2 μL, 3 μL, 4 μL, 5 μL, 6 μL, 7 μL, 8 μL, 9 μL, 10 μL, 20 μL, 30 μL, 40 μL, 50 μL, 60 μL, 70 μL, 80 μL, 90 μL, 100 μL, 150 μL, 200 μL, 250 μL, 300 μL, 350 μL, 400 μL, 450 μL, 500 μL, or more, guide nucleic acids, or nucleic acids encoding the guide nucleic acids, as described herein. In some embodiments, systems, compositions, methods, kits, devices, and solutions comprise 1 nM, 2 nM, 3 nM, 4 nM, 5 nM, 6 nM, 7 nM, 8 nM, 9 nM, 10 nM, 20 nM, 30 nM, 40 nM, 50 nM, 60 nM, 70 nM, 80 nM, 90 nM, 100 nM, 150 nM, 200 nM, 250 nM, 300 nM, 350 nM, 400 nM, 450 nM, 500 nM, or more, guide nucleic acids, or nucleic acids encoding the guide nucleic acids, as described herein. In some embodiments, systems, compositions, methods, kits, devices, and solutions comprise 1 μM, 2 μM, 3 μM, 4 μM, 5 μM, 6 μM, 7 μM, 8 μM, 9 μM, 10 μM, 20 μM, 30 μM, 40 μM, 50 μM, 60 μM, 70 μM, 80 μM, 90 μM, 100 μM, 150 μM, 200 μM, 250 μM, 300 μM, 350 μM, 400 μM, 450 μM, 500 μM, or more, guide nucleic acids, or nucleic acids encoding the guide nucleic acids, as described herein. In some embodiments, systems, compositions, methods, kits, devices, and solutions comprise 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 150 mM, 200 mM, 250 mM, 300 mM, 350 mM, 400 mM, 450 mM, 500 mM, or more, guide nucleic acids, or nucleic acids encoding the guide nucleic acids, as described herein.

In some embodiments, systems are used for detecting the presence of a target nucleic acid associated with or causative of a disease, such as cancer, a genetic disorder, or an infection. In some embodiments, systems are useful for phenotyping, genotyping, or determining ancestry. Unless specified otherwise, systems comprise kits. In some embodiments, systems comprising kits are referred to as kits. Unless specified otherwise, systems comprise devices. In some embodiments, systems comprising devices are referred to as devices. In some embodiments, systems described herein are provided in the form of a companion diagnostic assay or device, a point-of-care assay or device, or an over-the-counter diagnostic assay/device. Unless specified otherwise, systems described herein are used in methods for detecting the presence of a target nucleic acid.

In some embodiments, reagents and effector proteins of various systems are provided in a reagent chamber or on a support medium. Alternatively, the reagent and/or effector protein, in some embodiments, are contacted with the reagent chamber or the support medium by the individual using the system. An exemplary reagent chamber is a test well or container. In some embodiments, the opening of the reagent chamber is large enough to accommodate the support medium. Optionally, the system comprises a buffer and a dropper. In some embodiments, the buffer is provided in a dropper bottle for ease of dispensing. In some embodiments, the dropper is disposable and transfer a fixed volume. In some embodiments, the dropper is used to place a sample into the reagent chamber or on the support medium.

In some embodiments, the systems described herein comprising an engineered guide nucleic acid, wherein the engineered guide nucleic acid is capable of hybridizing to a target sequence in a target nucleic acid, and wherein the target nucleic acid is any one of: a naturally occurring eukaryotic sequence, an engineered eukaryotic sequence, a fragment of a naturally occurring eukaryotic sequence, a fragment of an engineered eukaryotic sequence, and combinations thereof. In some embodiments, the target nucleic acid is isolated from a human cell.

Also described herein are systems comprising an engineered polypeptide, or a recombinant nucleic acid encoding the engineered polypeptide, wherein the engineered polypeptide comprises an amino acid sequence that is at least 85% identical to any one of the sequences set forth in TABLE 1 or TABLE 1.2. In some embodiments, the recombinant nucleic acid encoding the polypeptide is a nucleic acid expression vector. In some embodiments, the nucleic acid expression vector is a viral vector. In some embodiments, the nucleic acid expression vector is an adeno associated viral (AAV) vector. In some embodiments, the nucleic acid expression vector encodes at least one engineered guide nucleic acid.

In some embodiments, the systems described herein are present in a single composition.

### System Solutions

In general, system components comprise a composition or solution in which the activity of an effector protein occurs. Often, the solution comprises or consists essentially of a buffer. In some embodiments, the solution or buffer comprises a buffering agent, a salt, a crowding agent, a detergent, a reducing agent, a competitor, or a combination thereof. Often the buffer is the primary component or the basis for the solution in which the activity occurs. Thus, concentrations for components of buffers described herein (e.g., buffering agents, salts, crowding agents, detergents, reducing agents, and competitors) are the same or essentially the same as the concentration of these components in the solution in which the activity occurs. In some embodiments, a buffer is required for cell lysis activity or viral lysis activity.

In some embodiments, systems comprise a buffer, wherein the buffer comprise at least one buffering agent. Exemplary buffering agents include HEPES, TRIS, MES, ADA, PIPES, ACES, MOPSO, BIS-TRIS propane, BES, MOPS, TES, DISO, Trizma, TRICINE, GLY-GLY, HEPPS, BICINE, TAPS, A MPD, A MPSO, CHES, CAPSO, AMP, CAPS, IB1, TCEP, EGTA, Tween 20, KC1, KOH, MgCl2, glycerol, or any combination thereof. In some embodiments, a buffer comprises Tris-HCl pH8.8, VLB, EGTA, CH3COOH, TCEP, IsoAmp®, (NH4)2SO4, KCl, MgSO4, Tween20, KOAc, MgOAc, BSA, phosphate, citrate, acetate, imidazole, or any combination thereof. In some embodiments, the concentration of the buffering agent in the buffer is 1 mM to 200 mM. In some embodiments, a buffer compatible with an effector protein comprises a buffering agent at a concentration of 10 mM to 30 mM. In some embodiments, a buffer compatible with an effector protein comprises a buffering agent at a concentration of about 20 mM. In some embodiments, a buffering agent provides a pH for the buffer or the solution in which the activity of the effector protein occurs. In some embodiments, the pH is in range of from 3 to 4, 3.5 to 4.5, 4 to 5, 4.5 to 5.5, 5 to 6, 5.5 to 6.5, 6 to 7, 6.5 to 7.5, 7 to 8, 7.5 to 8.5, 8 to 9, 8.5 to 9.5, 9 to 10, or 9.5 to 10.5.

In some embodiments, systems comprise a solution, wherein the solution comprises one or more salt. Accordingly, in some embodiments, the salt is one or more salt(s) selected from a magnesium salt, a zinc salt, a potassium salt, a calcium salt, and a sodium salt. In some embodiments, the salt is a combination of two or more salts. For example, in some embodiments, the salt is a combination of two or more salts selected from a magnesium salt, a zinc salt, a potassium salt, a calcium salt and a sodium salt. In some embodiments, the salt is magnesium acetate. In some embodiments, the salt is magnesium chloride. In some embodiments, the salt is potassium acetate. In some embodiments, the salt is potassium nitrate. In some embodiments, the salt is zinc chloride. In embodiments, the salt is sodium chloride. In some embodiments, the salt is potassium chloride. In some embodiments, the concentration of the one or more salt in the solution is about 0.001 mM to about 500 mM. In some embodiments, the concentration of the salt is about 0.001 mM to about 400 mM. In some embodiments, the concentration of the salt is about 0.001 mM to about 300 mM. In some embodiments, the concentration of the salt is about 0.001 mM to about 200 mM. In some embodiments, the concentration of the salt is about 0.001 mM to about 100 mM. In some embodiments, the concentration of the salt is about 0.001 mM to about 10 mM. In some embodiments, the concentration of the salt is about 0.01 mM to about 500 mM. In some embodiments, the concentration of the salt is about 0.01 mM to about 400 mM. In some embodiments, the concentration of the salt is about 0.01 mM to about 300 mM. In some embodiments, the concentration of the salt is about 0.01 mM to about 200 mM. In some embodiments, the concentration of the salt is about 0.01 mM to about 100 mM. In some embodiments, the concentration of the salt is about 0.01 mM to about 10 mM. In some embodiments, the concentration of the salt is about 0.1 mM to about 500 mM. In some embodiments, the concentration of the salt is about 0.1 mM to about 400 mM. In some embodiments, the concentration of the salt is about 0.1 mM to about 300 mM. In some embodiments, the concentration of the salt is about 0.1 mM to about 200 mM. In some embodiments, the concentration of the salt is about 0.1 mM to about 100 mM. In some embodiments, the concentration of the salt is about 0.1 mM to about 10 mM. In some embodiments, the concentration of the salt is about 1 mM to about 500 mM. In some embodiments, the concentration of the salt is about 1 mM to about 400 mM. In some embodiments, the concentration of the salt is about 1 mM to about 300 mM. In some embodiments, the concentration of the salt is about 1 mM to about 200 mM. In some embodiments, the concentration of the salt is about 1 mM to about 100 mM. In some embodiments, the concentration of the salt is about 1 mM to about 10 mM. In some embodiments, the concentration of the salt is about 10 mM to about 500 mM. In some embodiments, the concentration of the salt is about 10 mM to about 400 mM. In some embodiments, the concentration of the salt is about 10 mM to about 300 mM. In some embodiments, the concentration of the salt is about 10 mM to about 200 mM. In some embodiments, the concentration of the salt is about 10 mM to about 100 mM. In some embodiments, the concentration of the salt is about 100 mM to about 500 mM. In some embodiments, the concentration of the salt is about 100 mM to about 400 mM. In some embodiments, the concentration of the salt is about 100 mM to about 300 mM. In some embodiments, the concentration of the salt is about 100 mM to about 200 mM. In some embodiments, the salt is potassium acetate and the concentration of salt in the solution is about 100 mM. In some embodiments, the salt is potassium acetate or sodium chloride and the concentration of salt in the solution is about 200 mM. In some embodiments, the salt is potassium acetate or sodium chloride and the concentration of salt in the solution is about 100 mM to about 200 mM.

In some embodiments, systems comprise a solution, wherein the solution comprises at least one crowding agent. In some embodiments, a crowding agent reduces the volume of solvent available for other molecules in the solution, thereby increasing the effective concentrations of said molecules. Exemplary crowding agents include glycerol and bovine serum albumin. In some embodiments, the crowding agent is glycerol. In some embodiments, the concentration of the crowding agent in the solution is 0.01% (v/v) to 10% (v/v). In some embodiments, the concentration of the crowding agent in the solution is 0.5% (v/v) to 10% (v/v).

In some embodiments, systems comprise a solution, wherein the solution comprises at least one detergent. Exemplary detergents include Tween, Triton-X, and IGEPAL. In some embodiments, a solution comprises Tween, Triton-X, or any combination thereof. In some embodiments, a solution comprises Triton-X. In some embodiments, a solution comprises IGEPAL CA-630. In some embodiments, the concentration of the detergent in the solution is 2% (v/v) or less. In some embodiments, the concentration of the detergent in the solution is 1% (v/v) or less. In some embodiments, the concentration of the detergent in the solution is 0.00001% (v/v) to 0.01% (v/v). In some embodiments, the concentration of the detergent in the solution is about 0.01% (v/v).

In some embodiments, systems comprise a solution, wherein the solution comprises at least one reducing agent. Exemplary reducing agents comprise dithiothreitol (DTT), 8-mercaptoethanol (BME), or tris(2-carboxyethyl) phosphine (TCEP). In some embodiments, the reducing agent is DTT. In some embodiments, the concentration of the reducing agent in the solution is 0.01 mM to 100 mM. In some embodiments, the concentration of the reducing agent in the solution is 0.1 mM to 10 mM. In some embodiments, the concentration of the reducing agent in the solution is 0.5 mM to 2 mM. In some embodiments, the concentration of the reducing agent in the solution is 0.01 mM to 100 mM. In some embodiments, the concentration of the reducing agent in the solution is 0.1 mM to 10 mM. In some embodiments, the concentration of the reducing agent in the solution is about 1 mM.

In some embodiments, systems comprise a solution, wherein the solution comprises a competitor. In general, competitors compete with the target nucleic acid or the reporter nucleic acid for cleavage by the effector protein or a dimer thereof. Exemplary competitors include heparin, and imidazole, and salmon sperm DNA. In some embodiments, the concentration of the competitor in the solution is 1 μg/mL to 100 μg/mL. In some embodiments, the concentration of the competitor in the solution is 40 μg/mL to 60 μg/mL.

In some embodiments, systems comprise a solution, wherein the solution comprises a co-factor. In some embodiments, the co-factor allows an effector protein or a multiprotein complex thereof to perform a function, including pre-crRNA processing and/or target nucleic acid cleavage. In some embodiments, the suitability of a cofactor for an effector protein or a multiprotein complex thereof may be assessed, such as by methods based on those described by Sundaresan et al. (Cell Rep. 2017 Dec. 26; 21(13): 3728-3739). In some embodiments, an effector or a multiprotein complex thereof forms a complex with a co-factor. In some embodiments, the co-factor is a divalent metal ion. In some embodiments, the divalent metal ion is selected from Mg²⁺, Mn²⁺, Zn²⁺, Ca²⁺, Cu²⁺. In some embodiments, the divalent metal ion is Mg²⁺. In some embodiments, the co-factor is Mg²⁺.

In some embodiments, systems, and compositions for use with systems comprise a catalytic reagent for signal improvement or enhancement. In some embodiments, the catalytic reagent enhances signal generation via hydrolysis of inorganic pyrophosphates. In some embodiments, catalytic reagents enhance signal generation via enhancement of DNA/RNA replication. In some embodiments, catalytic reagents enhance signal amplification via revival of ions (e.g., Mg2+) in a buffer, thereby enhancing the function of an effector protein. In some embodiments, the catalytic reagent for signal improvement comprises an enzyme. In some embodiments, the catalytic reagent for signal improvement are particularly useful in amplification and/or detection reactions as described herein. Other exemplary reagents useful for amplification and/or detection reactions (i.e., amplification and detection reagents, respectively) are described throughout herein.

Any of the systems, methods, or compositions described herein comprise a catalytic reagent or the use thereof. In some embodiments, compositions comprise about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, or 10 enzyme unit (U) of a catalytic reagent per 10 μL of solution. In some embodiments, a catalytic reagent is present in a composition at a concentration of 0.125 Units, 0.5 Units, 0.25 Units, 1.0 Units, 2.0 Units, 2.5 Units, or 4 Units per discrete reaction volume. In some embodiments, a catalytic reagent is provided in a system separately from a buffer provided in the system. In some embodiments, systems comprise a buffer, wherein a catalytic reagent is provided in the buffer.

In some embodiments, a catalytic reagent improves the signal to noise ratio of an effector protein-based detection reaction. In some embodiments, a catalytic reagent improves overall signal (e.g., fluorescence of a cleaved reporter). In some embodiments, a catalytic reagent improves signal by a factor, wherein the signal is indicative of the presence of a target nucleic acid. In some embodiments, the factor is at least about 1.1, at least about 2, at least about 3, at least about 4, at least about 5, at least about 6, at least about 7, at least about 8, at least about 9, or at least about 10.

Also provided herein are reagents for: detection reactions, nuclease purification, cell lysis, in vitro transcription reactions, amplification reactions, reverse and transcription reactions. In some embodiments, systems, compositions, and/or solutions described herein comprise one or more of: detection reagents, nuclease purification reagents, cell lysis reagents, in vitro transcription reagents, amplification reagents, reverse transcription reagents, and combinations thereof. In some embodiments, any such reagents suitable with the solutions, compositions, methods, systems, devices, and/or kits described herein are used for achieving one or more of the foregoing described reactions. In some embodiments, reagents provided herein are used with any other solution components described herein, including buffers, amino acids or derivatives thereof, chaotropes, chelators, cyclodextrins, inhibitors, ionic liquids, linkers, metals, non-detergent sulfobetaines, organic acids, osmolytes, peptides, polyamides, polymers, polyols, polyols and salts, salts, or combinations thereof.

### Detection Reagents/Components and Reporters

In some embodiments, systems disclosed herein comprise detection reagents to facilitate detection of nucleic acids as described herein. Non-limiting examples of detection reagents include a reporter nucleic acid, a detection moiety, and additional polypeptides. In some embodiments, the detection reagent is operably linked to an effector protein described herein such that a detection event occurs upon contacting the detection reagent and effector protein with a target nucleic acid. Upon the occurrence of the detection event, a signal (e.g., a detectable signal or detectable product) can be generated thereby indicating detection of the target nucleic acid. In some embodiments, any suitable detection reagent may be used. Accordingly, in some embodiments, the detection reagent comprises a nucleic acid (which, in some embodiments, is referred to herein as a detection or reporter nucleic acid), a detection moiety, an additional polypeptide, or a combination thereof. Other detection reagents include buffers, reverse transcriptase mix, a catalytic reagent, and a stain. Any reagents suitable with the detection reactions, events, and signals described herein are useful as detection reagents for the systems, compositions, methods, kits, devices, and solutions provided herein. In some embodiments, detection reagents detect a nucleic acid in a sample.

In some embodiments, systems, compositions, methods, kits, devices, and solutions comprise 0.01 μL, 0.02 μL, 0.03 μL, 0.04 μL, 0.05 μL, 0.06 μL, 0.07 μL, 0.08 μL, 0.09 μL, 0.1 μL, 0.2 μL, 0.3 μL, 0.4 μL, 0.5 μL, 0.6 μL, 0.7 μL, 0.8 μL, 0.9 μL, 1 μL, 2 μL, 3 μL, 4 μL, 5 μL, 6 μL, 7 μL, 8 μL, 9 μL, 10 μL, 20 μL, 30 μL, 40 μL, 50 μL, 60 μL, 70 μL, 80 μL, 90 μL, 100 μL, 150 μL, 200 μL, 250 μL, 300 μL, 350 μL, 400 μL, 450 μL, 500 μL, or more of each detection reagent as described herein. In some embodiments, systems, compositions, methods, kits, devices, and solutions comprise 1 nM, 2 nM, 3 nM, 4 nM, 5 nM, 6 nM, 7 nM, 8 nM, 9 nM, 10 nM, 20 nM, 30 nM, 40 nM, 50 nM, 60 nM, 70 nM, 80 nM, 90 nM, 100 nM, 150 nM, 200 nM, 250 nM, 300 nM, 350 nM, 400 nM, 450 nM, 500 nM, or more of each detection reagent as described herein. In some embodiments, systems, compositions, methods, kits, devices, and solutions comprise 1 μM, 2 μM, 3 μM, 4 μM, 5 μM, 6 μM, 7 μM, 8 μM, 9 μM, 10 μM, 20 μM, 30 μM, 40 μM, 50 μM, 60 μM, 70 μM, 80 μM, 90 μM, 100 μM, 150 μM, 200 μM, 250 μM, 300 μM, 350 μM, 400 μM, 450 μM, 500 μM, or more of each detection reagent as described herein. In some embodiments, systems, compositions, methods, kits, devices, and solutions comprise 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 150 mM, 200 mM, 250 mM, 300 mM, 350 mM, 400 mM, 450 mM, 500 mM, or more of each detection reagent as described herein.

In some embodiments, detection reagents detect a nucleic acid in a sample. In some embodiments, nucleic acid amplification of the target nucleic acid improves at least one of sensitivity, specificity, or accuracy of the assay in detecting the target nucleic acid. Accordingly, in some embodiments, nucleic acid detection involves PCR or isothermal nucleic acid amplification, providing improved sensitive, specific, or rapid detection. In some embodiments, the reagents or components for nucleic acid detection comprise recombinases, primers, polypeptides, buffers, and signal reagents suitable for a detection reaction.

In some embodiments, systems described herein comprise a PCR tube, a PCR well or a PCR plate. In some embodiments, the wells of the PCR plate are pre-aliquoted with the reagent for detecting a nucleic acid, as well as a guide nucleic acid, an effector protein, a multiprotein complex, an amplification reagent, or any combination thereof. In some embodiments, the pre-aliquoted guide nucleic acid targeting a target sequence, and an effector protein that is activated when complexed with the guide nucleic acid and the target sequence. Accordingly, in some embodiments, a user adds a sample of interest to a well of the pre-aliquoted PCR plate.

In some embodiments, nucleic acid detection is performed in a nucleic acid detection region on a support medium, or sample interface. Alternatively, or in combination, the nucleic acid detection is performed in a reagent chamber, and the resulting sample is applied to the support medium, sample interface, or surface within a reagent chamber.

In some embodiments, the reporter nucleic acid is cleaved by the activated nuclease, thereby generating a detectable signal. Accordingly, in some embodiments, a user adds a sample of interest to a well of the pre-aliquoted PCR plate and measure for the detectable signal with a fluorescent light reader or a visible light reader.

In some embodiments, detection reaction of nucleic acid as described herein is performed for no greater than 1, 2, 3, 4, 5, 6, 7, 8, 9, 10, 11, 12, 13, 14, 15, 16, 17, 18, 19, 20, 25, 30, 40, 50, or 60 minutes, or any value 1 to 60 minutes. In some embodiments, the detection reaction is performed for 1 to 60, 5 to 55, 10 to 50, 15 to 45, 20 to 40, or 25 to 35 minutes. In some embodiments, the detection reaction is performed at a temperature of around 20-45° C. In some embodiments, the detection reaction is performed at a temperature no greater than 20° C., 25° C., 30° C., 35° C., 37° C., 40° C., 45° C., or any value 20° C. to 45° C. In some embodiments, the detection reaction is performed at a temperature of at least 20° C., 25° C., 30° C., 35° C., 37° C., 40° C., or 45° C., or any value 20° C. to 45° C. In some embodiments, the detection reaction is performed at a temperature of 20° C. to 45° C., 25° C. to 40° C., 30° C. to 40° C., or 35° C. to 40° C.

In some embodiments, the reagents or components for detecting a nucleic acid are, for example, consistent for use within various fluidic devices disclosed herein for detection of a target nucleic acid within the sample, wherein the fluidic device may comprise multiple pumps, valves, reservoirs, and chambers for sample preparation, amplification of a target nucleic acid within the sample, mixing with an effector protein, and detection of a detectable signal arising from cleavage of nucleic acids by an effector protein within the fluidic system itself. These reagents are compatible with the samples, solutions, compositions, systems, devices, fluidic devices, methods of detection, and support mediums as described herein for detection of an ailment, such as a disease, cancer, or genetic disorder, or genetic information, such as for phenotyping, genotyping, or determining ancestry. The reagents described herein for detecting a disease, cancer, or genetic disorder comprise a guide nucleic acid targeting the target nucleic acid segment indicative of a disease, cancer, or genetic disorder.

In some embodiments, systems disclosed herein comprise a reporter. By way of non-limiting and illustrative example, a reporter comprises a single stranded nucleic acid and a detection moiety (e.g., a labeled single stranded RNA reporter), wherein the nucleic acid is cleaved by an effector protein (e.g., a CRISPR/Cas protein as disclosed herein) or a multiprotein complex thereof, releasing the detection moiety, and generating a detectable signal or a detectable product. In some embodiments, cleavage of the reporter is effective to produce a detectable product comprising a detectable moiety or a detectable signal. The effector proteins disclosed herein, activated upon hybridization of a guide nucleic acid to a target nucleic acid, cleaves the reporter. Cleavage of a reporter produces different types of signals (e.g., a detectable signal). In some embodiments, cleavage of the reporter produces a calorimetric signal, a potentiometric signal, an amperometric signal, an optical signal, or a piezo-electric signal. Various devices and/or sensors can be used to detect these different types of signals, which indicate whether a target nucleic acid, is present in the sample. The sensors usable to detect such signals can include, for example, optical sensors (e.g., imaging devices for detecting fluorescence or optical signals with various wavelengths and frequencies), electric potential sensors, surface plasmon resonance (SPR) sensors, interferometric sensors, or any other type of sensor suitable for detecting calorimetric signals, potentiometric signals, amperometric signals, optical signals, or piezo-electric signals.

As used herein, a reporter comprises a nucleic acid (e.g., RNA and/or DNA). In some embodiments, a reporter is double-stranded. In some embodiments, a reporter is single-stranded. In some embodiments, a reporter comprises a protein that generates a detectable signal or signal. In some embodiments, a reporter is operably linked to the protein that generates a signal. In some embodiments, a signal is a calorimetric, potentiometric, amperometric, optical (e.g., fluorescent, colorimetric, etc.), or piezo-electric signal. In some embodiments, the reporter comprises a detection moiety. In some embodiments, the reporter is configured to release a detection moiety or generate a signal indicative of a presence or absence of the target nucleic acid. For example, the signal can indicate a presence of the target nucleic acid in the sample, and an absence of the signal can indicate an absence of the target nucleic acid in the sample. In some embodiments, suitable detectable labels and/or moieties provide a signal. In some embodiments, non-limiting example of a suitable detectable label and/or moiety comprises an enzyme, a radioisotope, a member of a specific binding pair; a fluorophore; a fluorescent protein; and a quantum dot.

In some embodiments, the reporter comprises a detection moiety and a quenching moiety. In some embodiments, the reporter comprises a cleavage site, wherein the detection moiety is located at a first site on the reporter and the quenching moiety is located at a second site on the reporter, wherein the first site and the second site are separated by the cleavage site. Sometimes the quenching moiety is a fluorescence quenching moiety. In some embodiments, the quenching moiety is 5′ to the cleavage site and the detection moiety is 3′ to the cleavage site. In some embodiments, the detection moiety is 5′ to the cleavage site and the quenching moiety is 3′ to the cleavage site. Sometimes the quenching moiety is at the 5′ terminus of the nucleic acid of a reporter. Sometimes the detection moiety is at the 3′ terminus of the nucleic acid of a reporter. In some embodiments, the detection moiety is at the 5′ terminus of the nucleic acid of a reporter. In some embodiments, the quenching moiety is at the 3′ terminus of the nucleic acid of a reporter.

Suitable fluorescent proteins include, but are not limited to, green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz (TYFP), Venus, Citrine, mCitrine, GFPuv, destabilised EGFP (dEGFP), destabilised ECFP (dECFP), destabilised EYFP (dEYFP), mCFPm, Cerulean, T-Sapphire, CyPet, YPet, mKO, HcRed, t-HcRed, DsRed, DsRed2, DsRed-monomer, J-Red, dimer2, t-dimer2(12), mRFP1, pocilloporin, Renilla GFP, Monster GFP, paGFP, Kaede protein and kindling protein, Phycobiliproteins and Phycobiliprotein conjugates including B-Phycoerythrin, R-Phycoerythrin and Allophycocyanin. Suitable enzymes include, but are not limited to, horseradish peroxidase (HRP), alkaline phosphatase (AP), beta-galactosidase (GAL), glucose-6-phosphate dehydrogenase, beta-N-acetylglucosaminidase, β-glucuronidase, invertase, Xanthine Oxidase, firefly luciferase, and glucose oxidase (GO).

In some embodiments, the detection moiety comprises an invertase. In some embodiments, the substrate of the invertase comprises sucrose. In some embodiments, a DNS reagent that is included in the system for producing a colorimetric change when the invertase converts sucrose to glucose. In some embodiments, the reporter nucleic acid and invertase are conjugated using a heterobifunctional linker by sulfo-SMCC chemistry.

In some embodiments, suitable fluorophores provide a detectable fluorescence signal in the same range as 6-Fluorescein (Integrated DNA Technologies), IRDye 700 (Integrated DNA Technologies), TYE 665 (Integrated DNA Technologies), Alex Fluor 594 (Integrated DNA Technologies), or ATTO TM 633 (NHS Ester) (Integrated DNA Technologies). Non-limiting examples of fluorophores are fluorescein amidite, 6-Fluorescein, IRDye 700, TYE 665, Alex Fluor 594, or ATTO TM 633 (NHS Ester). In some embodiments, the fluorophore comprises an infrared fluorophore. In some embodiments, the fluorophore emits fluorescence in the range of 500 nm and 720 nm. In some embodiments, the fluorophore emits fluorescence at a wavelength of 700 nm or higher. In other embodiments, the fluorophore emits fluorescence at about 665 nm. In some embodiments, the fluorophore emits fluorescence in the range of 500 nm to 520 nm, 500 nm to 540 nm, 500 nm to 590 nm, 590 nm to 600 nm, 600 nm to 610 nm, 610 nm to 620 nm, 620 nm to 630 nm, 630 nm to 640 nm, 640 nm to 650 nm, 650 nm to 660 nm, 660 nm to 670 nm, 670 nm to 680 nm, 690 nm to 690 nm, 690 nm to 700 nm, 700 nm to 710 nm, 710 nm to 720 nm, or 720 nm to 730 nm. In some embodiments, the fluorophore emits fluorescence in the range 450 nm to 750 nm, 500 nm to 650 nm, or 550 to 650 nm.

In some embodiments, systems may comprise a quenching moiety. In some embodiments, a quenching moiety is chosen based on its ability to quench the detection moiety. In some embodiments, a quenching moiety comprises a non-fluorescent fluorescence quencher. In some embodiments, a quenching moiety quenches a detection moiety that emits fluorescence in the range of 500 nm and 720 nm. In some embodiments, a quenching moiety quenches a detection moiety that emits fluorescence in the range of 500 nm and 720 nm. In some embodiments, the quenching moiety quenches a detection moiety that emits fluorescence at a wavelength of 700 nm or higher. In other embodiments, the quenching moiety quenches a detection moiety that emits fluorescence at about 660 nm or about 670 nm. In some embodiments, the quenching moiety quenches a detection moiety that emits fluorescence in the range of 500 to 520, 500 to 540, 500 to 590, 590 to 600, 600 to 610, 610 to 620, 620 to 630, 630 to 640, 640 to 650, 650 to 660, 660 to 670, 670 to 680, 690 to 690, 690 to 700, 700 to 710, 710 to 720, or 720 to 730 nm. In some embodiments, the quenching moiety quenches a detection moiety that emits fluorescence in the range 450 nm to 750 nm, 500 nm to 650 nm, or 550 to 650 nm. In some embodiments, a quenching moiety quenches fluorescein amidite, 6-Fluorescein, IRDye 700, TYE 665, Alex Fluor 594, or ATTO TM 633 (NHS Ester). In some embodiments, a quenching moiety comprises Iowa Black RQ, Iowa Black FQ or IRDye QC-1 Quencher. In some embodiments, a quenching moiety quenches fluorescein amidite, 6-Fluorescein (Integrated DNA Technologies), IRDye 700 (Integrated DNA Technologies), TYE 665 (Integrated DNA Technologies), Alex Fluor 594 (Integrated DNA Technologies), or ATTO TM 633 (NHS Ester) (Integrated DNA Technologies). In some embodiments, a quenching moiety comprises Iowa Black RQ (Integrated DNA Technologies), Iowa Black FQ (Integrated DNA Technologies) or IRDye QC-1 Quencher (LiCor). Any of the quenching moieties described herein may be from any commercially available source, may be an alternative with a similar function, a generic, or a non-tradename of the quenching moieties listed.

In some embodiments, the generation of a detectable product or detectable signal from the release of the detection moiety indicates that cleavage by the effector protein has occurred and that the sample contains the target nucleic acid. In some embodiments, the detection moiety comprises a fluorescent dye. Sometimes the detection moiety comprises a fluorescence resonance energy transfer (FRET) pair. In some embodiments, the detection moiety comprises an infrared (IR) dye. In some embodiments, the detection moiety comprises an ultraviolet (UV) dye. Alternatively, or in combination, the detection moiety comprises a protein. Sometimes the detection moiety comprises an antigen. Sometimes the detection moiety comprises a biotin. Sometimes the detection moiety comprises at least one of avidin or streptavidin. In some embodiments, the detection moiety comprises a polysaccharide, a polymer, or a nanoparticle. In some embodiments, the detection moiety comprises a gold nanoparticle or a latex nanoparticle.

In some embodiments, a detection moiety comprises any moiety that generates a detectable product or detectable signal upon cleavage of the reporter by the effector protein. In some embodiments, the detectable product comprises a detectable unit generated from the detectable moiety and that emits a detectable signal as described herein. In some embodiments, the detectable product further comprises a detectable label, a fluorophore, a reporter, or a combination thereof. In some embodiments, the detectable product comprises RNA, DNA, or both. In some embodiments, the detectable product is configured to generate a signal indicative of the presence or absence of the target nucleic acid in, for instance, a cell or a sample.

In some embodiments, a detection moiety comprises any moiety that generates a calorimetric, potentiometric, amperometric, optical (e.g., fluorescent, colorimetric, etc.), or piezo-electric signal. A nucleic acid of a reporter, sometimes, is protein-nucleic acid that generates a calorimetric, potentiometric, amperometric, optical (e.g., fluorescent, colorimetric, etc.), or piezo-electric signal upon cleavage of the nucleic acid. Often a calorimetric signal is heat produced after cleavage of the nucleic acids of a reporter. Sometimes, a calorimetric signal is heat absorbed after cleavage of the nucleic acids of a reporter. A potentiometric signal, for example, is electrical potential produced after cleavage of the nucleic acids of a reporter. In some embodiments, an amperometric signal comprises movement of electrons produced after the cleavage of nucleic acid of a reporter. Often, the signal is an optical signal, such as a colorimetric signal or a fluorescence signal. An optical signal is, for example, a light output produced after the cleavage of the nucleic acids of a reporter. Sometimes, an optical signal is a change in light absorbance between before and after the cleavage of nucleic acids of a reporter. Often, a piezo-electric signal is a change in mass between before and after the cleavage of the nucleic acid of a reporter.

In some embodiments, the detectable signal comprises a colorimetric signal or a signal visible by eye. In some embodiments, the detectable signal may be fluorescent, electrical, chemical, electrochemical, or magnetic. In some embodiments, the first detection signal is generated by interaction of the detection moiety to the capture molecule in the detection region, where the first detection signal indicates that the sample contained the target nucleic acid. Sometimes systems detect more than one type of target nucleic acid, wherein the system comprises more than one type of guide nucleic acid and more than one type of reporter nucleic acid. In some embodiments, the detectable signal is generated directly by the cleavage event. Alternatively, or in combination, the detectable signal is generated indirectly by the signal event. Sometimes the detectable signal is not a fluorescent signal. In some embodiments, the detectable signal comprises a colorimetric or color-based signal. In some embodiments, the detected target nucleic acid is identified based on its spatial location on the detection region of the support medium. In some embodiments, the second detectable signal is generated in a spatially distinct location than the first generated signal.

In some embodiments, the reporter nucleic acid is a single-stranded nucleic acid sequence comprising ribonucleotides. In some embodiments, the nucleic acid of a reporter comprises a single-stranded nucleic acid sequence comprising at least one ribonucleotide. In some embodiments, the nucleic acid of a reporter is a single-stranded nucleic acid comprising at least one ribonucleotide residue at an internal position that functions as a cleavage site. In some embodiments, the nucleic acid of a reporter comprises at least 2, at least 3, at least 4, at least 5, at least 6, at least 7, at least 8, at least 9, or at least 10 ribonucleotide residues at an internal position. In some embodiments, the nucleic acid of a reporter comprises from 2 to 10, from 3 to 9, from 4 to 8, or from 5 to 7 ribonucleotide residues at an internal position. Sometimes the ribonucleotide residues are continuous. Alternatively, the ribonucleotide residues are interspersed in between non-ribonucleotide residues. In some embodiments, the nucleic acid of a reporter has only ribonucleotide residues. In some embodiments, the nucleic acid of a reporter has only DNA residues. In some embodiments, the nucleic acid comprises nucleotides resistant to cleavage by the effector protein described herein. In some embodiments, the nucleic acid of a reporter comprises synthetic nucleotides. In some embodiments, the nucleic acid of a reporter comprises at least one ribonucleotide residue and at least one non-ribonucleotide residue.

In some embodiments, the nucleic acid of a reporter comprises at least one uracil ribonucleotide. In some embodiments, the nucleic acid of a reporter comprises at least two uracil ribonucleotides. Sometimes the nucleic acid of a reporter has only uracil ribonucleotides. In some embodiments, the nucleic acid of a reporter comprises at least one adenine ribonucleotide. In some embodiments, the nucleic acid of a reporter comprises at least two adenine ribonucleotides. In some embodiments, the nucleic acid of a reporter has only adenine ribonucleotides. In some embodiments, the nucleic acid of a reporter comprises at least one cytosine ribonucleotide. In some embodiments, the nucleic acid of a reporter comprises at least two cytosine ribonucleotides. In some embodiments, the nucleic acid of a reporter comprises at least one guanine ribonucleotide. In some embodiments, the nucleic acid of a reporter comprises at least two guanine ribonucleotides. In some embodiments, a nucleic acid of a reporter comprises a single unmodified ribonucleotide. In some embodiments, a nucleic acid of a reporter comprises only unmodified DNAs.

In some embodiments, the nucleic acid of a reporter is 5 to 20, 5 to 15, 5 to 10, 7 to 20, 7 to 15, or 7 to 10 nucleotides in length. In some embodiments, the nucleic acid of a reporter is 3 to 20, 4 to 10, 5 to 10, or 5 to 8 nucleotides in length. In some embodiments, the nucleic acid of a reporter is 5 to 12 nucleotides in length. In some embodiments, the reporter nucleic acid is at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 nucleotides in length. In some embodiments, the reporter nucleic acid is 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length.

In some embodiments, systems comprise a plurality of reporters. In some embodiments, the plurality of reporters comprise a plurality of signals. In some embodiments, systems comprise at least 2, at least 3, at least 4, at least 5, at least 6, at least 7, at least 8, at least 9, at least 10, at least 11, at least 12, at least 13, at least 14, at least 15, at least 20, at least 30, at least 40, or at least 50 reporters. In some embodiments, there are 2 to 50, 3 to 40, 4 to 30, 5 to 20, or 6 to 10 different reporters.

In some embodiments, systems comprise an effector protein and a reporter nucleic acid configured to undergo trans cleavage by the effector protein. In some embodiments, trans cleavage of the reporter generates a signal from the reporter or alter a signal from the reporter. In some embodiments, the signal is an optical signal, such as a fluorescence signal or absorbance band. In some embodiments, trans cleavage of the reporter alters the wavelength, intensity, or polarization of the optical signal. For example, in some embodiments, the reporter comprises a fluorophore and a quencher, such that trans cleavage of the reporter separates the fluorophore and the quencher thereby increasing a fluorescence signal from the fluorophore. Herein, in some embodiments, detection of reporter cleavage to determine the presence of a target nucleic acid is referred to as ‘DETECTR’. In some embodiments described herein is a method of assaying for a target nucleic acid in a sample comprising contacting the target nucleic acid with an effector protein, a non-naturally occurring guide nucleic acid that hybridizes to a segment of the target nucleic acid, and a reporter nucleic acid, and assaying for a change in a signal, wherein the change in the signal is produced by cleavage of the reporter nucleic acid.

In the presence of a large amount of non-target nucleic acids, in some embodiments, an activity of an effector protein (e.g., an effector protein as disclosed herein) is inhibited. This is because the activated effector proteins collaterally cleave any nucleic acids. In some embodiments, if total nucleic acids are present in large amounts, they outcompete reporters for the effector proteins. In some embodiments, systems comprise an excess of reporter(s), such that when the system is operated and a solution of the system comprising the reporter is combined with a sample comprising a target nucleic acid, the concentration of the reporter in the combined solution-sample is greater than the concentration of the target nucleic acid. In some embodiments, the sample comprises amplified target nucleic acid. In some embodiments, the sample comprises an unamplified target nucleic acid. In some embodiments, the concentration of the reporter is greater than the concentration of target nucleic acids and non-target nucleic acids. In some embodiments, the non-target nucleic acids from the original sample, either lysed or unlysed. In some embodiments, the non-target nucleic acids comprise byproducts of amplification. In some embodiments, systems comprise a reporter wherein the concentration of the reporter in a solution 1.5 fold, at least 2 fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 16 fold, at least 17 fold, at least 18 fold, at least 19 fold, at least 20 fold, at least 30 fold, at least 40 fold, at least 50 fold, at least 60 fold, at least 70 fold, at least 80 fold, at least 90 fold, at least 100 fold excess of total nucleic acids.

### Amplification Reagents/Components

In some embodiments, systems described herein comprise a reagent or component for amplifying a nucleic acid. Non-limiting examples of reagents for amplifying a nucleic acid include polymerases, primers, and nucleotides. In some embodiments, systems comprise reagents for nucleic acid amplification of a target nucleic acid in a sample. In some embodiments, nucleic acid amplification of the target nucleic acid improves at least one of sensitivity, specificity, or accuracy of the assay in detecting the target nucleic acid. In some embodiments, nucleic acid amplification is isothermal nucleic acid amplification, providing for the use of the system or system in remote regions or low resource settings without specialized equipment for amplification. In some embodiments, amplification of the target nucleic acid increases the concentration of the target nucleic acid in the sample relative to the concentration of nucleic acids that do not correspond to the target nucleic acid.

In some embodiments, the reagents for nucleic acid amplification comprise a recombinase, a primer, an oligonucleotide primer, an activator, a deoxynucleoside triphosphate (dNTP), a ribonucleoside tri-phosphate (rNTP), a single-stranded DNA binding (SSB) protein, Rnase inhibitor, water, a polymerase, reverse transcriptase mix, or a combination thereof that is suitable for an amplification reaction. Non-limiting examples of amplification reactions are transcription mediated amplification (TMA), helicase dependent amplification (HDA), or circular helicase dependent amplification (cHDA), strand displacement amplification (SDA), recombinase polymerase amplification (RPA), loop mediated amplification (LAMP), exponential amplification reaction (EXPAR), rolling circle amplification (RCA), ligase chain reaction (LCR), simple method amplifying RNA targets (SMART), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), and improved multiple displacement amplification (IMDA).

Such amplification reactions, in some embodiments, also used in combination with reverse transcription (RT) of an RNA of interest. Accordingly, also provided herein are reagents for both the reverse transcription and amplification of nucleic acids. In some embodiments, systems, compositions, methods, kits, devices, and solutions comprise 0.01 μL, 0.02 μL, 0.03 μL, 0.04 μL, 0.05 μL, 0.06 μL, 0.07 μL, 0.08 μL, 0.09 μL, 0.1 μL, 0.2 μL, 0.3 μL, 0.4 μL, 0.5 μL, 0.6 μL, 0.7 μL, 0.8 μL, 0.9 μL, 1 μL, 2 μL, 3 μL, 4 μL, 5 μL, 6 μL, 7 μL, 8 μL, 9 μL, 10 μL, 20 μL, 30 μL, 40 μL, 50 μL, 60 μL, 70 μL, 80 μL, 90 μL, 100 μL, 150 μL, 200 μL, 250 μL, 300 μL, 350 μL, 400 μL, 450 μL, 500 μL, or more of each amplification described herein. In some embodiments, systems, compositions, methods, kits, devices, and solutions comprise 1 nM, 2 nM, 3 nM, 4 nM, 5 nM, 6 nM, 7 nM, 8 nM, 9 nM, 10 nM, 20 nM, 30 nM, 40 nM, 50 nM, 60 nM, 70 nM, 80 nM, 90 nM, 100 nM, 150 nM, 200 nM, 250 nM, 300 nM, 350 nM, 400 nM, 450 nM, 500 nM, or more of each amplification reagent as described herein. In some embodiments, systems, compositions, methods, kits, devices, and solutions comprise 1 μM, 2 μM, 3 μM, 4 μM, 5 μM, 6 μM, 7 μM, 8 μM, 9 μM, 10 μM, 20 μM, 30 μM, 40 μM, 50 μM, 60 μM, 70 μM, 80 μM, 90 μM, 100 μM, 150 μM, 200 μM, 250 μM, 300 μM, 350 μM, 400 μM, 450 μM, 500 μM, or more of each amplification reagent as described herein. In some embodiments, systems, compositions, methods, kits, devices, and solutions comprise 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 150 mM, 200 mM, 250 mM, 300 mM, 350 mM, 400 mM, 450 mM, 500 mM, or more of each amplification reagent as described herein.

In some embodiments, systems described herein comprise a PCR tube, a PCR well or a PCR plate. In some embodiments, the wells of the PCR plate are pre-aliquoted with the reagent for amplifying a nucleic acid, as well as a guide nucleic acid, an effector protein, a multiprotein complex, or any combination thereof. In some embodiments, the wells of the PCR plate are pre-aliquoted with a guide nucleic acid targeting a target sequence, an effector protein that is activated when complexed with the guide nucleic acid and the target sequence, an effector protein that is activated when complexed with the guide nucleic acid and the target sequence, and at least one population of a single stranded reporter nucleic acid comprising a detection moiety. In some embodiments, a user thus adds the biological sample of interest to a well of the pre-aliquoted PCR plate and measure for the detectable signal with a fluorescent light reader or a visible light reader.

In some embodiments, systems comprise a PCR plate; a guide nucleic acid targeting a target sequence; an effector protein that is activated when complexed with the guide nucleic acid and the target sequence; and a single stranded reporter nucleic acid comprising a detection moiety, wherein the reporter nucleic acid is cleaved by the activated nuclease, thereby generating a detectable signal.

In some embodiments, systems described herein comprise a support medium; a guide nucleic acid targeting a target sequence; and an effector protein that is activated when complexed with the guide nucleic acid and the target sequence. In some embodiments, nucleic acid amplification is performed in a nucleic acid amplification region on the support medium. Alternatively, or in combination, the nucleic acid amplification is performed in a reagent chamber, and the resulting sample is applied to the support medium.

In some embodiments, a system described herein for modifying a target nucleic acid comprises a PCR plate; a guide nucleic acid targeting a target sequence; and an effector protein that is activated when complexed with the guide nucleic acid and the target sequence. In some embodiments, the wells of the PCR plate are pre-aliquoted with the guide nucleic acid targeting a target sequence, and an effector protein that is activated when complexed with the guide nucleic acid and the target sequence. In some embodiments, a user thus adds the biological sample of interest to a well of the pre-aliquoted PCR plate.

Often, the nucleic acid amplification is performed for no greater than 1, 2, 3, 4, 5, 6, 7, 8, 9, 10, 11, 12, 13, 14, 15, 16, 17, 18, 19, 20, 25, 30, 40, 50, or 60 minutes, or any value 1 to 60 minutes. In some embodiments, the amplification reaction is performed for 1 to 60, 5 to 55, 10 to 50, 15 to 45, 20 to 40, or 25 to 35 minutes. In some embodiments, the amplification reaction is performed at a temperature of around 20-45° C. In some embodiments, the amplification reaction is performed at a temperature no greater than 20° C., 25° C., 30° C., 35° C., 37° C., 40° C., 45° C., 50° C., 55° C., 60° C. or any value 20° C. to 60° C. In some embodiments, the amplification reaction is performed at a temperature of at least 20° C., 25° C., 30° C., 35° C., 37° C., 40° C., 45° C., 50° C., 55° C., 60° C. or any value 20° C. to 60° C. In some embodiments, the amplification reaction is performed at a temperature of 20° C. to 45° C., 25° C. to 40° C., 30° C. to 40° C., 35° C. to 40° C., 40° C. to 45° C., 45° C. to 50° C., 50° C. to 55° C., 55° C. to 60° C.

In some embodiments, systems comprise primers for amplifying a target nucleic acid to produce an amplification product comprising the target nucleic acid and a PAM. In some embodiments, at least one of the primers comprise the PAM that is incorporated into the amplification product during amplification. The compositions for amplification of target nucleic acids and methods of use thereof, as described herein, are compatible with any of the methods disclosed herein including methods of assaying for at least one base difference (e.g., assaying for a SNP or a base mutation) in a target nucleic acid, methods of assaying for a target nucleic acid that lacks a PAM by amplifying the target nucleic acid to introduce a PAM, and compositions used in introducing a PAM by amplification into the target nucleic acid.

### Additional System Components

In some embodiments, systems include a package, carrier, or container that is compartmentalized to receive one or more containers such as vials, or tubes, each of the container(s) comprising one of the separate elements to be used in a method described herein. Suitable containers include, for example, test wells, bottles, vials, syringes, and test tubes. In some embodiments, the containers are formed from a variety of materials such as glass, plastic, or polymers. In some embodiments, the system or systems described herein contain packaging materials. Examples of packaging materials include, but are not limited to, pouches, blister packs, bottles, tubes, bags, containers, bottles, and any packaging material suitable for intended mode of use.

In some embodiments, systems described herein include labels listing contents and/or instructions for use, or package inserts with instructions for use. In some embodiments, the systems include a set of instructions and/or a label is on or associated with the container. In some embodiments, the label is on a container when letters, numbers or other characters forming the label are attached, molded, or etched into the container itself; a label is associated with a container when it is present within a receptacle or carrier that also holds the container (e.g., as a package insert). In some embodiments, the label is used to indicate that the contents are to be used for a specific therapeutic application. In some embodiments, the label indicates directions for use of the contents, such as in the methods described herein. In some embodiments, after packaging the formed product and wrapping or boxing to maintain a sterile barrier, the product is terminally sterilized by heat sterilization, gas sterilization, gamma irradiation, or by electron beam sterilization. Alternatively, in some embodiments, the product is prepared and packaged by aseptic processing.

In some embodiments, systems comprise a solid support. In some embodiments, an RNP or effector protein is attached to a solid support. In some embodiments, the solid support comprises an electrode or a bead. In some embodiments, the bead comprises a magnetic bead. Upon cleavage, the RNP is liberated from the solid support and interacts with other mixtures. For example, upon cleavage of the nucleic acid of the RNP, the effector protein of the RNP flows through a chamber into a mixture comprising a substrate. When the effector protein meets the substrate, a reaction occurs, such as a colorimetric reaction, which is then detected. As another example, the protein is an enzyme substrate, and upon cleavage of the nucleic acid of the enzyme substrate-nucleic acid, the enzyme flows through a chamber into a mixture comprising the enzyme. When the enzyme substrate meets the enzyme, a reaction occurs, such as a calorimetric reaction, which is then detected.

### Certain System Conditions

In some embodiments, systems and methods are employed under certain conditions that enhance an activity of the effector protein relative to alternative conditions, as measured by a detectable signal released from cleavage of a reporter in the presence of the target nucleic acid. In some embodiments, the detectable signal is generated at about the rate of trans cleavage of a reporter nucleic acid. In some embodiments, the reporter nucleic acid is a homopolymeric reporter nucleic acid comprising 5 to 20 consecutive adenines (e.g., SEQ ID NO: 114), 5 to 20 consecutive thymines (e.g., SEQ ID NO: 115), 5 to 20 consecutive cytosines (e.g., SEQ ID NO: 116), or 5 to 20 consecutive guanines (e.g., SEQ ID NO: 117). In some embodiments, the reporter is an RNA-FQ reporter.

In some embodiments, effector proteins disclosed herein recognize, bind, or are activated by, different target nucleic acids having different sequences, but are active toward the same reporter nucleic acid, allowing for facile multiplexing in a single assay having a single ssRNA-FQ reporter.

In some embodiments, systems are employed under certain conditions that enhance trans cleavage activity of an effector protein. In some embodiments, under certain conditions, trans cleavage occurs at a rate of at least 0.005 mmol/min, at least 0.01 mmol/min, at least 0.05 mmol/min, at least 0.1 mmol/min, at least 0.2 mmol/min, at least 0.5 mmol/min, or at least 1 mmol/min. In some embodiments, systems and methods are employed under certain conditions that enhance cis cleavage activity of the effector protein.

Certain conditions that may enhance the activity of an effector protein include a certain salt presence or salt concentration of the solution in which the activity occurs. For example, in some embodiments, cis cleavage activity of an effector protein is inhibited or halted by a high salt concentration. In some embodiments, the salt comprises a magnesium salt, a zinc salt, a potassium salt, a calcium salt, a lithium salt, an ammonium salt, or a sodium salt. In some embodiments, the salt is magnesium acetate. In some embodiments, the salt is magnesium chloride. In some embodiments, the salt is potassium acetate. In some embodiments, the salt is potassium nitrate. In some embodiments, the salt is zinc chloride. In embodiments, the salt is sodium chloride. In some embodiments, the salt is potassium chloride. In some embodiments, the salt is lithium acetate. In some embodiments, the salt is ammonium sulfate. In some embodiments, the salt concentration is less than 150 mM, less than 125 mM, less than 100 mM, less than 75 mM, less than 50 mM, or less than 25 mM. In some embodiments, the salt concentration is more than 1 mM, but less than 150 mM, less than 125 mM, less than 100 mM, less than 75 mM, less than 50 mM, or less than 25 mM. In some embodiments, the salt concentration is more than 10 mM, but less than 150 mM, less than 125 mM, less than 100 mM, less than 75 mM, less than 50 mM, or less than 25 mM. In some embodiments, the salt is potassium acetate or sodium chloride and the concentration of salt in the solution is about 200 mM. In some embodiments, the salt is potassium acetate or, sodium chloride, lithium acetate, or ammonium sulfate and the concentration of salt in the solution is about 100 mM to about 200 mM.

Certain conditions that may enhance the activity of an effector protein include the pH of a solution in which the activity. For example, in some embodiments, increasing pH enhances trans cleavage activity. For example, in some embodiments, the rate of trans cleavage activity increases with increase in pH up to pH9. In some embodiments, the pH is about 7, about 7.1, about 7.2, about 7.3, about 7.4, about 7.5, about 7.6, about 7.7, about 7.8, about 7.9, about 8, about 8.1, about 8.2, about 8.3, about 8.4, about 8.5, about 8.6, about 8.7, about 8.8, about 8.9, or about 9. In some embodiments, the pH is 7 to 7.5, 7.5 to 8, 8 to 8.5, 8.5 to 9, or 7 to 8.5. In some embodiments, the pH is less than 7. In some embodiments, the pH is greater than 7.

Certain conditions that may enhance the activity of an effector protein includes the temperature at which the activity is performed. In some embodiments, the temperature is about 25° C. to about 80° C. In some embodiments, the temperature is about 20° C. to about 40° C., about 30° C. to about 50° C., or about 40° C. to about 60° C. In some embodiments, the temperature is about 25° C., about 30° C., about 35° C., about 40° C., about 45° C., about 50° C., about 55° C., about 60° C., about 65° C., about 70° C., about 75° C., or about 80° C.

### XI. Devices

Disclosed herein are devices for modifying and/or detecting target nucleic acid. In some embodiments, devices comprise components comprising one or more of: compositions described herein; systems described herein; other components or appurtenances as described herein; or combinations thereof.

### Device Components

In general, device components comprise a structural component as well as sample components, including compositions, solutions, and systems described herein. Often, a sample component comprises or consists essentially of compositions, or systems described herein. Additional device components may comprise one or more hydrogels or surfaces with immobilized reporters. In some embodiments, a device's sample component may be contained in at least one structural device component, such as a sample interface, which may be in fluid communication with a chamber. In some embodiments, the sample interface is fluidically connected to a chamber. By way of non-limiting example, a device's sample component may be simultaneously contained in a sample interface and a chamber. In some embodiments, by being in fluid communication with a chamber, a device's sample component may flow from the sample interface to the chamber. In some embodiments, a device's sample component may flow from the sample interface into a chamber by way of the fluid connection. In some embodiments, a reporter is immobilized to a surface or support medium within the chamber, which may be a hydrogel. In some embodiments, a chamber comprises more than one effector protein type.

In some embodiments, the devices described herein comprise a plurality of hydrogels each comprising reporter molecules (e.g., in order to facilitate multiplexing and/or improve signal). In some embodiments, a first hydrogel comprises a shape different from a shape of a second hydrogel. In some embodiments, the first hydrogel comprises a plurality of first reporter molecules different from a plurality of second reporter molecules of the second hydrogel. In some embodiments, the reporters are the same in the first and second hydrogels. In some embodiments, the first hydrogel comprises a circular shape, a square shape, a star shape, or any other shape distinguishable from a shape of the second hydrogel. In some embodiments, the plurality of first reporter molecules each comprise a sequence cleavable by an effector protein-guide nucleic acid complex comprising a first effector protein and a first guide nucleic acid. In some embodiments, the plurality of second reporter molecules each comprise a sequence not cleavable by the effector protein-guide nucleic acid complex.

Any of the devices described herein comprise a plurality of hydrogels each comprising reporter molecules. For example, a first hydrogel comprises a plurality of first reporter molecules different from a plurality of second reporter molecules of a second hydrogel. In some embodiments, the plurality of first reporter molecules each comprise a first fluorescent moiety, wherein the first fluorescent moiety is different than second fluorescent moieties of in each of the plurality of second reporter molecules. In some embodiments, the plurality of first reporter molecules each comprise a sequence cleavable by a first effector protein-guide nucleic acid complex comprising a first effector protein and a first guide nucleic acid. In some embodiments, the plurality of second reporter molecules each comprise a sequence cleavable by a second effector protein-guide nucleic acid complex comprising a second effector protein and a second guide nucleic acid.

Any of the devices described herein comprise at least about 2 hydrogels, at least about 3 hydrogels, at least about 4 hydrogels, at least about 5 hydrogels, at least about 6 hydrogels, at least about 7 hydrogels, at least about 8 hydrogels, at least about 9 hydrogels, at least about 10 hydrogels, at least about 20 hydrogels, at least about 30 hydrogels, at least about 40 hydrogels, at least about 50 hydrogels, at least about 60 hydrogels, at least about 70 hydrogels, at least about 80 hydrogels, at least about 90 hydrogels, at least about 100 hydrogels, at least about 200 hydrogels, at least about 300 hydrogels, at least about 400 hydrogels, at least about 500 hydrogels, at least about 600 hydrogels, at least about 700 hydrogels, at least about 800 hydrogels, at least about 900 hydrogels, at least about 1,000 hydrogels.

Any of the devices described herein comprise one or more compartments, chambers, channels, or locations comprising the one or more hydrogels or surfaces. In some embodiments, two or more of the compartments or chambers are in fluid communication, optical communication, thermal communication, or any combination thereof with one another. In some embodiments, two or more compartments or chambers are arranged in a sequence. In some embodiments, two or more compartments or chambers are arranged in parallel. In some embodiments, two or more compartments or chambers are arranged in sequence, parallel, or both. In some embodiments, one or more compartments or chambers comprise a well. In some embodiments, one or more compartments or chambers comprise a flow strip. In some embodiments, one or more compartments or chambers comprise a heating element.

Any of the devices described herein comprise a sample interface, which are in fluid communication with a valve and/or a chamber, or comprising configuration to be fluidically connected to a valve and/or a chamber. In some embodiments, a device's sample component lows from the sample interface, through a valve, and into a chamber. In some embodiments, a valve disposed between the sample interface and the chamber comprises configuration to selectively resist flow or permit flow. In some embodiments, a chamber comprises configuration to comprise compositions, systems, one or more reagents for amplification (i.e., amplification reagents), one or more reagents for detection (i.e., detection reagents), one or more cell lysis reagents, one or more nucleic acid purification reagents, or combinations thereof. In some embodiments, a chamber and/or a valve comprises configuration to be thermally connected to a heating element. In some embodiments, each of the valves of the plurality of valves is thermally connected to a heating element. In some embodiments, each of the valves is filled with a material configured to change between liquid and solid phases when heated by a heating element.

Any of the devices described herein comprise a plurality of chambers and/or a plurality of valves configured to be fluidically connected. In some embodiments, a plurality of valves comprise configuration to restrict flow in a first direction through channels and/or sample interface. In some embodiments, a first subset of the plurality of valves comprise configuration to restrict flow in a first direction through one or more channels towards the sample interface. In some embodiments, a plurality of valves comprise configuration to restrict flow in a second direction through channels and/or a reaction chamber. In some embodiments, a second subset of the plurality of valves comprise configuration to selectively permit flow in a second direction through one or more channels towards the reaction chamber. In some embodiments, a plurality of valves configured to comprise a valve inlet channel and/or a valve outlet channel. In some embodiments, each of the valves of the plurality of valves comprises a valve inlet channel and a valve outlet channel. In some embodiments, a cross-sectional area of the valve inlet channel is less than a cross-sectional area of the corresponding valve outlet channel. In some embodiments, a plurality of valves comprise configuration to simultaneously or independently be in an open state or a closed state. In some embodiments, a plurality of valves comprising a first valve and a second valve. In some embodiments, a plurality of valves comprise configuration to physically, fluidically, or thermally isolate a first portion of a sample from a second portion of a sample when a first valve and a second valve are in a closed state.

In some embodiments, a plurality of chambers comprise a first chamber and a second chamber, wherein the second chamber is disposed between the sample interface and the first chamber. In some embodiments, a second chamber is disposed fluidically downstream of the sample interface and the first chamber. In some embodiments, a second chamber is disposed upstream of the sample interface and the first chamber. In some embodiments, a first chamber is disposed to be fluidically connected to a detection region. In some embodiments, a second chamber comprises one or more reagents for amplification, one or more cell lysis reagents, one or more nucleic acid purification reagents. In some embodiments, a detection region comprises an array, one or more lateral flow strips, a detection tray, a detection region comprising a capture antibody, or combinations thereof.

In some embodiments, the device comprises one or more lateral flow assay strips in a detection region disposed downstream of a reaction chamber. In some embodiments, the device comprises one or more lateral flow assay strips in a detection region which, in some embodiments, is brought into fluid communication with the reaction chamber. Each lateral flow assay strip contains one or more detection regions or spots, where each detection region or spot contains a different type of capture antibody. In some embodiments, each lateral flow assay strip contains a different type of capture antibody. In some embodiments, each capture antibody type specifically binds to a particular label type of a reporter. In some embodiments, the reaction chamber comprises one or more guide nucleic acids (e.g., crRNAs), and/or effector proteins described herein.

Also described herein are devices comprising: (a) a polypeptide, or a nucleic acid encoding the polypeptide, and an engineered guide nucleic acid, or a nucleic acid that encodes the engineered guide nucleic acid; (b) a polypeptide, or a nucleic acid encoding the polypeptide, and an engineered guide nucleic acid comprising a crRNA; (c) an mRNA encoding a polypeptide, and an engineered guide nucleic acid; (d) one or more recombinant expression vectors comprising: i) a nucleic acid encoding a polypeptide; and ii) an engineered guide nucleic acid; or (e) one or more recombinant expression vectors comprising: i) a nucleic acid encoding a polypeptide; and ii) an engineered guide nucleic acid comprising a crRNA; wherein the polypeptide comprises an amino acid sequence that is at least 85% identical to any one of the sequences set forth in TABLE 1 or TABLE 1.2. In some embodiments, the device is used in diagnosis of a disease or disorder associated with a nucleic acid sequence modification in a viral genome, a prokaryotic genome, or a eukaryotic genome. In some embodiments, the device is used in diagnosis of a disease or disorder associated with a non-wild type gene, a gene comprising a non-wild type reading frame, a gene comprising one or more mutations, abnormal processing upon transcription of a gene, combinations thereof.

In general, the buffers described herein are compatible for use in the devices described herein (e.g., pneumatic valve devices, sliding valve devices, rotating valve devices, lateral flow devices, and microfluidic devices). In some embodiments, the device is a microfluidic device. In some embodiments, the device is a handheld device. In some embodiments, the device is a point-of-need device. In some embodiments, the device comprises any one of the device configurations described herein. In some embodiments, the device comprises one or more parts of any one of the device configurations described herein.

Generally, a sample comprises one or more target nucleic acids and a chamber (e.g., a reaction chamber) comprises one or more of: effector proteins, guide nucleic acids, and reporters comprising a nucleic acid and a detection moiety. In some embodiments, a sample flows from a sample interface into a chamber by way of the fluid connection wherein the sample interacts with the components of the compositions, systems, and solutions contained therein. In some embodiments, upon interacting, an effector protein and a guide nucleic acid form an effector protein-guide nucleic acid complex (e.g., an RNP). In some embodiments, an effector protein becomes activated after binding of a guide nucleic acid, that is complexed with the effector protein, with a target nucleic acid, and the activated effector protein cleaves the target nucleic acid, which can result in a trans cleavage activity. Trans cleavage activity can be non-specific cleavage of nearby single-stranded nucleic acids by the activated effector protein, such as trans cleavage of the nucleic acid (e.g., a detector nucleic acid) with a detection moiety of the reporter. Once the target nucleic acid is cleaved by the activated effector protein, the detection moiety can be released or separated from the reporter and can directly or indirectly generate a detectable signal. The reporter and/or the detection moiety can be immobilized on a support medium, such as a surface or hydrogel within the device. Often the detection moiety is at least one of a fluorophore, a dye, a polypeptide, or a nucleic acid. Sometimes the detection moiety binds to a capture molecule on the support medium or hydrogel to be immobilized. The detectable signal can be visualized on the support medium or hydrogel to assess the presence or concentration of one or more target nucleic acids associated with an ailment, such as a disease, cancer, or genetic disorder.

Any of the devices described herein are compatible with any of the compositions, systems, kits, or methods disclosed herein, including methods of detecting and treating a disease or disorder. By way of non-limiting example, in some embodiments, the devices described herein are used in diagnosis of a disease or disorder. In some embodiments, the devices described herein are used in detection of any one of the diseases or disorders recited in TABLE 5 and TABLE 5.1. In some embodiments, the devices described herein are used in detection of any one of a disease or disorder associated with a gene selected from a viral genome, a prokaryotic genome, or a eukaryotic genome. In some embodiments, the devices described herein are used in detection of any one of a disease or disorder associated with a non-wild type gene, a gene comprising a non-wild type reading frame, a gene comprising one or more mutations, or abnormal processing upon transcription of a gene. In some embodiments, the devices described herein are used in detection of a modified nucleic acid sequence associated with a disease or disorder associated gene. Also, by way of non-limiting example, the devices described herein are compatible with detection of a nucleic acid sequence selected from a viral genome, a prokaryotic genome, or a eukaryotic genome.

### Microfluidic Devices

Disclosed herein are microfluidic devices and uses thereof, e.g., use for detection of target nucleic acids. Devices described herein can be used for an effector protein-based detection (e.g., DETECTR) assay. In some embodiments, the devices are compatible with multiplex lateral flow detection. In some embodiments, the devices are configured to perform one or more of the reactions described herein (e.g., amplification, detection, etc.) in separate chambers. In some embodiments, isolating portions of a liquid sample for detection of different target nucleic acids facilitates multiplexing (e.g., by air gaps separating the liquid contents of various chambers during a reaction). In some embodiments, the devices described herein can be used in combination with enzyme-based methods for signal amplification of a binding event between one or more effector protein probes and one or more target nucleic acids. In some embodiments, signal detection is performed on the device (e.g., in a reaction chamber, or in a detection chamber connected to the reaction chamber). In some embodiments, the device is configured to allow removal of the contents of a reaction chamber to perform a signal detection step. Methods for signal detection compatible with the devices are also disclosed herein.

In some embodiments, a microfluidic device comprising: a sample interface configured to receive a sample; and a chamber fluidically connected to the sample interface. In some embodiments, the sample for use in the microfluidic device comprises one or more nucleic acids, for example, one or more target nucleic acids. In some embodiments, the sample for use in the microfluidic device comprises one or more target nucleic acids, for example, one or more target nucleic acids associated to nucleic acids as set forth in TABLE 4 and TABLE 4.1 herein. Suitable sample conditions are also described herein and include suitable target copy numbers, solutions, and the like.

In some embodiments, a chamber of the microfluidic device comprises one or more components of the compositions, systems, or solutions described herein. In some embodiments, the chamber of the microfluidic device comprises one or more of: an effector protein, a guide nucleic acid, a reporter, or any combination thereof. In some embodiments, a chamber or channel further comprises a reporter comprising a nucleic acid and a detection moiety.

In some embodiments, microfluidic devices described herein comprise a sample interface configured to receive a sample, wherein the sample comprises one or more target nucleic acids; and a chamber fluidically connected to the sample interface, wherein the chamber comprises an effector protein described herein, an engineered guide nucleic acid described herein, a reporter comprising a nucleic acid and a detection moiety, and reagents (e.g., detection reagents); wherein the sample comprising the target nucleic acids, the effector protein, the engineered guide nucleic acid, and the reporter are able to interact by way of the fluid connection. As described herein, the effector protein and the engineered guide nucleic acid contained in the chamber form an activated complex upon hybridization of the engineered guide nucleic acid to a target sequence of a target nucleic acid and wherein the nucleic acid of the reporter is a cleavage substrate of the activated complex. The activated complex is capable cleaving the nucleic acid of the reporter (i.e., the detection event), releasing the detection moiety and thereby allowing it to generate a detectable signal. Accordingly, in some embodiments, a target nucleic acid is detected in the form of a signal (i.e., a detectable signal or detectable product) as a result of the reaction between the sample liquid, or a portion thereof, and the effector protein-based reagents, as described herein.

In some embodiments, the microfluidic device further comprises a valve disposed between the sample interface and the chamber. In some embodiments, the valve is configured to selectively resist flow, or permit flow of the sample components and the chamber components as described herein. Nonlimiting examples of valves include phase-change valves, wax valves, capillary valves, electrostatic valves, check valves, sliding valves, rotary valves, pneumatic valves, vacuum valves, pinch valves, and burst valves.

In some embodiments, the chamber further comprises one or more: amplification reagents, detection reagents, cell lysis reagents, and/or nucleic acid purification reagents. Amplification reagents, detection reagents, cell lysis reagents, and/or nucleic acid purification reagents are described herein, for example, in the Examples. In some embodiments, the chamber further comprises a polymerase, for example a DNA polymerase or an RNA polymerase. Other suitable polymerases are described herein.

In some embodiments, the chamber is a first chamber and the microfluidic device further comprising a second chamber comprising one or more: amplification reagents, detection reagents, cell lysis reagents, and/or nucleic acid purification reagents. In some embodiments, the second chamber or channel is disposed between the sample interface and the first chamber, wherein the second chamber or channel is disposed downstream of the sample interface and the first chamber, wherein the second chamber or channel is disposed upstream of the sample interface and the first chamber.

In some embodiments, the microfluidic device further comprises a detection region fluidically connected to the first chamber. In some embodiments, the detection region comprises an array, one or more lateral flow strips, a detection tray, a detection region comprising a capture antibody, or combinations thereof.

In some embodiments, the microfluidic device comprising: (i) a sample interface configured to receive a sample; (ii) a first actuator configured to provide positive pressure to an upstream portion of the device proximate to the sample interface; (iii) a second actuator configured to provide negative pressure to a downstream portion of the device distal to the sample interface; (iv) a heating channel in fluid communication with the sample interface and first actuator, wherein the heating channel comprises a first portion in a first plane and a second portion in a second plane parallel to the first plane; (v) a first heating element disposed between and in thermal contact with the first and second portions of the heating channel; (vi) a reagent mixing chamber fluidically connected downstream of the heating channel by a first valve; (vii) a plurality of reaction chambers in fluid communication with the second actuator; (viii) a channel multiplexing unit fluidically connected downstream of the mixing chamber by a second valve, wherein the channel multiplexing unit comprises a main channel and a plurality of side channels, wherein each of the side channels is in fluid communication with one inlet of a plurality of inlets positioned along the main channel, and wherein each of the side channels comprises an outlet in fluid communication with one of the plurality of reaction chambers; (ix) a first valve actuator configured to actuate the first valve; and (x) a second valve actuator configured to actuate the second valve. In some embodiments, each of the first and second valves comprise a valve inlet channel and a valve outlet channel, wherein a cross-sectional area of the valve inlet channel is less than a cross-sectional area of the corresponding valve outlet channel. In some embodiments, the main channel is serially divided into a plurality of subchannel portions and a tolerance channel, wherein the tolerance channel is positioned at a distal end of the main channel. In some embodiments, each of the plurality of reaction chambers is connected to the downstream portion by each of a plurality of hydrophobic venting membranes. In some embodiments, each valve actuator comprises a heating element in thermal contact with the respective valve. In some embodiments, the first actuator and second actuator are operably connected such that actuation of the first actuator triggers actuation of the second actuator. In some embodiments, the first actuator is operably connected to a trigger or a timing mechanism that controls the heating elements and valve actuators.

It is understood that description of device configurations or components herein, also describes microfluidic device configuration or components provided herein and vice versa. Accordingly, in some embodiments, the microfluidic device comprises any one of the device configurations described herein. In still some embodiments, the microfluidic device comprises one or more parts of any one of the device configurations described herein.

Also described herein are microfluidic devices comprising: (a) a sample interface configured to receive a sample comprising nucleic acids; and (b) a chamber fluidically connected to the sample interface; wherein the chamber comprises a polypeptide and an engineered guide nucleic acid, wherein the polypeptide comprises an amino acid sequence that is at least 85% identical to any one of the sequences set forth in TABLE 1 or TABLE 1.2. In some embodiments, the chamber further comprises a reporter comprising a nucleic acid and a detection moiety. In some embodiments, the polypeptide is effective to form an activated complex with the engineered guide nucleic acid upon hybridization of the engineered guide nucleic acid to a target sequence of a target nucleic acid and wherein the nucleic acid of the reporter is a cleavage substrate of the activated complex. In some embodiments, the reporter is immobilized to a surface within the chamber. In some embodiments, the nucleic acid of the reporter comprises a ribonucleotide, a deoxyribonucleotide, or combinations thereof. In some embodiments, the microfluidic devices described herein further comprising a valve disposed between the sample interface and the chamber, optionally wherein the valve is configured to selectively resist flow, or permit flow. In some embodiments, the chamber further comprises one or more reagents for amplification, one or more cell lysis reagents, one or more nucleic acid purification reagents. In some embodiments, the chamber further comprises a polymerase, a reverse transcriptase, or a combination thereof. In some embodiments, the chamber is a first chamber and the microfluidic device further comprising a second chamber comprising one or more reagents for amplification, one or more cell lysis reagents, one or more nucleic acid purification reagents. In some embodiments, the microfluidic devices described herein further comprising a channel comprising one or more reagents for amplification, one or more cell lysis reagents, one or more nucleic acid purification reagents. In some embodiments, the second chamber or channel is disposed between the sample interface and the first chamber, wherein the second chamber or channel is disposed downstream of the sample interface and the first chamber, wherein the second chamber or channel is disposed upstream of the sample interface and the first chamber. In some embodiments, the microfluidic devices described herein comprising a plurality of chambers fluidically connected to a plurality of valves. In some embodiments, a first subset of the plurality of valves are configured to restrict flow in a first direction through one or more channels towards the sample interface. In some embodiments, a second subset of the plurality of valves are configured to selectively permit flow in a second direction through one or more channels towards a reaction chamber. In some embodiments, a first valve and a second valve of the plurality of valves are configured to physically, fluidically, or thermally isolate a first portion of the sample from a second portion of the sample when the first valve and the second valve are in a closed state. In some embodiments, each valve of the plurality of valves comprises a valve inlet channel and a valve outlet channel; and wherein a cross-sectional area of the valve inlet channel is less than a cross-sectional area of the corresponding valve outlet channel. In some embodiments, each valve is thermally connected to a heating element. In some embodiments, each valve is filled with a material configured to change between liquid and solid phases when heated by a heating element. In some embodiments, the microfluidic devices described herein further comprising a detection region fluidically connected to the first chamber. In some embodiments, the detection region comprises an array, one or more lateral flow strips, a detection tray, a detection region comprising a capture antibody, or combinations thereof.

Also disclosed herein is a method of microfluidic devices described herein for target nucleic acid detection. In some embodiments, the method comprises applying a sample to the sample interface. In some embodiments, said applying forms a sample liquid. In some embodiments, the method can comprise sample collection. The method can further comprise sample preparation. In some embodiments, the method comprises using a physical filter to filter one or more particles from the sample that do not comprise the at least one analyte of interest (e.g., a target nucleic acid). In some embodiments, the method comprises lysing the sample before detecting the analyte. In some embodiments, the method comprises performing enzyme (e.g., Proteinase K or savinase) inactivation on the sample. In some embodiments, the method comprises performing heat inactivation on the sample. In some embodiments, the method comprises performing nucleic acid purification on the sample. In some embodiments, the method comprises contacting a plurality of sub-samples with a plurality of effector protein probes comprising different guide RNAs. In some embodiments, the sample is diluted with a buffer or a fluid or concentrated prior to application to the detection system.

In some embodiments, the sample can be provided manually to the device of the present disclosure. For example, a swab sample can be dipped into a solution and the sample/solution can be pipetted into the device. In other embodiments, the sample can be provided via an automated syringe. The automated syringe can be configured to control a flow rate at which the sample is provided to the device. The automated syringe can be configured to control a volume of the sample that is provided to the device over a predetermined period.

In some embodiments, the sample can be provided directly to the device of the present disclosure. For example, a swab sample can be inserted into a sample chamber on the device.

The sample can be prepared before one or more targets are detected within the sample. The sample preparation steps described herein can process a crude sample to generate a pure or purer sample. In some embodiments, sample preparation comprises one or more physical or chemical processes, including, for example, nucleic acid purification, lysis, binding, washing, and/or eluting. In certain embodiments, sample preparation can comprise the following steps, including sample collection, nucleic acid purification, heat inactivation, enzyme inactivation, and/or base/acid lysis.

In some embodiments, nucleic acid purification can be performed on the sample. Purification can comprise disrupting a biological matrix of a cell to release nucleic acids, denaturing structural proteins associated with the nucleic acids (nucleoproteins), inactivating nucleases that can degrade the isolated product (RNase and/or DNase), and/or removing contaminants (e.g., proteins, carbohydrates, lipids, biological or environmental elements, unwanted nucleic acids, and/or other cellular debris).

In some embodiments, lysis of a collected sample can be performed. Lysis can be performed using a protease (e.g., a Proteinase K or PK enzyme). Exemplary proteases include serine proteases (e.g., Proteinase K, Savinase®, trypsin, Protamex®, etc.), metalloproteinases (e.g., MMP-3, etc.), cysteine proteases (e.g., cathepsin B, papin, etc.), threonine proteases, aspartic proteases (e.g., renin, pepsin, cathepsin D, etc.), glutamic proteases, asparagine peptide lyases, or the like. In some cases, a solution of reagents can be used to lyse the cells in the sample and release the nucleic acids so that they are accessible to the effector protein. Active ingredients of the solution can be chaotropic agents, detergents, salts, and can be of high osmolality, ionic strength, and pH. Chaotropic agents or chaotropes are substances that disrupt the three-dimensional structure in macromolecules such as proteins, DNA, or RNA. In some embodiments, one example protocol comprises a 4 M guanidinium isothiocyanate, 25 mM sodium citrate·2H20, 0.5% (w/v) sodium lauryl sarcosinate, and 0.1 M β-mercaptoethanol), but numerous commercial buffers for different cellular targets can also be used. Alkaline buffers can also be used for cells with hard shells, particularly for environmental samples. Detergents such as sodium dodecyl sulphate (SDS) and cetyl trimethylammonium bromide (CTAB) can also be implemented to chemical lysis buffers. Cell lysis can also be performed by physical, mechanical, thermal or enzymatic means, in addition to chemically-induced cell lysis mentioned previously. In some cases, depending on the type of sample, nanoscale barbs, nanowires, acoustic generators, integrated lasers, integrated heaters, and/or microcapillary probes can be used to perform lysis.

In certain instances, heat inactivation can be performed on the sample. In some embodiments, a processed/lysed sample can undergo heat inactivation to inactivate, in the lysed sample, the proteins used during lysing (e.g., a PK enzyme or a lysing reagent) and/or other residual proteins in the sample (e.g., RNases, DNases, viral proteins, etc.). In some cases, a heating element integrated into the nucleic acid detection device can be used for heat-inactivation. The heating element can be powered by a battery or another source of thermal or electric energy that is integrated with the nucleic acid detection device.

In certain instances, enzyme inactivation can be performed on the sample. In some embodiments, a processed/lysed sample can undergo enzyme inactivation to inhibit or inactivate, in the lysed sample, the proteins used during lysing (e.g., a PK enzyme or a lysing reagent) and/or other residual proteins in the sample (e.g., RNases, DNases, etc.). In some cases, a solution of reagents can be used to inactivate one or more enzymes present in the sample. Enzyme inactivation can occur before, during, or after lysis, when lysis is performed. For example, in some embodiments, an RNase inhibitor is included as a lysis reagent to inhibit native RNases within the sample (which might otherwise impair target and/or reporter detection downstream). Exemplary RNase inhibitors include RNAse Inhibitor, Murine (NEB), RnaseIn Plus (Promega), Protector Rnase Inhibitor (Roche), SuperaseIn (Ambion), RiboLock (Thermo), Ribosafe (Bioline), or the like. Alternatively, or in combination, when a protease is used for sample lysis, a protease inhibitor can be applied to the lysed sample to inactivate the protease prior to contacting the sample nucleic acids to the effector protein. In some embodiments, additional application of heat is not be required to inhibit the protease (e.g., proteinase K) sufficiently to prevent additional activity of the protease (which could potentially impair effector protein activity downstream, in some embodiments). Exemplary protease inhibitors include AEBSF, antipain, aprotinin, bestatin, chymostatin, EDTA, leupeptin, pepstatin A, phosphoramidon, PMSF, soybean trypsin inhibitor, TPCK, or the like. In some embodiments, enzyme inactivation occurs before, during, after, or instead of heat inactivation.

In some embodiments, a target nucleic acid within the sample can undergo amplification before binding to a guide nucleic acid. The target nucleic acid within a purified sample can be amplified. In some instances, amplification can be accomplished using loop mediated amplification (LAMP), isothermal recombinase polymerase amplification (RPA), and/or polymerase chain reaction (PCR). In some instances, digital droplet amplification can used. Such nucleic acid amplification of the sample can improve at least one of a sensitivity, specificity, or accuracy of the detection of the target DNA. The reagents for nucleic acid amplification can comprise a recombinase, an oligonucleotide primer, a single-stranded DNA binding (SSB) protein, and a polymerase. The nucleic acid amplification can be transcription mediated amplification (TMA). Nucleic acid amplification can be helicase dependent amplification (HDA) or circular helicase dependent amplification (cHDA). In additional cases, nucleic acid amplification is strand displacement amplification (SDA). The nucleic acid amplification can be recombinase polymerase amplification (RPA). The nucleic acid amplification can be at least one of loop mediated amplification (LAMP) or the exponential amplification reaction (EXPAR). Nucleic acid amplification is, in some cases, by rolling circle amplification (RCA), ligase chain reaction (LCR), simple method amplifying RNA targets (SMART), single primer isothermal amplification (SPIA), multiple displacement amplification (MDA), nucleic acid sequence-based amplification (NASBA), hinge-initiated primer-dependent amplification of nucleic acids (HIP), nicking enzyme amplification reaction (NEAR), or improved multiple displacement amplification (IMDA).

In some embodiments, the method further comprises actuating flow of the sample liquid through the heating channel to each of the reaction chambers. In some embodiments, the method of actuating comprises actuating using a plunger, a spring-actuated plunger, or a spring mechanism. In some embodiments, the actuation is manual. In some embodiments, actuation is configured to move the sample from the sample interface to the heating region via manual actuation of the first actuator. In some embodiments, the device is configured to be operated manually without electrical power. In some embodiments, actuation is achieved using a pneumatic pump, a sliding device, a rotary device, and/or a lateral flow device.

In some embodiments, the method further comprises reacting the sample liquid with the effector protein, the guide nucleic acid, and the reporter. In some embodiments, the reagents described herein include a composition for improving detection signal strength, detection reaction time, detection reaction efficiency, stability, solubility, or the like. In some embodiments, the reaction generates a colorimetric signal, a fluorescent signal, an electrochemical signal, a chemiluminescent signal, or another type of signal. In some embodiments, the reaction induces color-change in substances.

In some embodiments, the method further comprises detecting a detectable signal when a target nucleic acid is present in the sample. The method can further comprise using an effector protein-based detection module to detect one or more targets (e.g., target sequences or target nucleic acids) in the sample. In some cases, the sample can be divided into a plurality of aliquots or subsamples to facilitate sample preparation and to enhance the detection capabilities of the devices of the present disclosure. In some cases, the sample is not divided into subsamples. In some embodiments, the detectable signal is a colorimetric signal, a fluorescent signal, an electrochemical signal, a chemiluminescent signal, or another type of signal. In some embodiments, the detectable signal is a color-change in substances. In some embodiments, detection is achieved using a sensor or detector. In some embodiments, detection is achieved either directly or indirectly. Additional illustrative embodiments for detecting a target nucleic acid using devices described herein are provided herein.

Further illustrative embodiments for devices (e.g., microfluidic devices) and methods of detecting target nucleic acids are described in further detail for example in WO2021207702, WO2021236850, WO2020142739, WO2021159020, WO2020028729, WO2020142754, WO2022133108, WO2020257356, WO2021243308, WO 2022061166, WO2022132833, and WO2022020393, the entire contents of each of which are incorporated herein by reference.

### XII. Kits

In some embodiments, compositions and/or system components are assembled in a kit. Accordingly, disclosed herein are kits for modifying, and/or detecting target nucleic acid. In some embodiments, kits are compatible with any methods disclosed herein, including methods used for detection, treatment, and/or diagnosis of a disease or disorder.

Any of the kits described herein are compatible with any of the compositions, systems, kits, or methods disclosed herein, including methods of detecting and treating a disease or disorder. By way of non-limiting example, in some embodiments, the kits described herein are used in diagnosis of a disease or disorder. In some embodiments, the kits described herein are used in detection of any one of the diseases or disorders recited in TABLE 5 and TABLE 5.1. By way of non-limiting example, in some embodiments, the kits described herein are used in diagnosis of a disease or disorder. In some embodiments, the kits described herein are used in detection of any one of the diseases or disorders recited in TABLE 5 and TABLE 5.1. In some embodiments, the kits described herein are used in detection of any one of a disease or disorder associated with a gene selected from a viral genome, a prokaryotic genome, or a eukaryotic genome. In some embodiments, the kits described herein are used in detection of any one of a disease or disorder associated with a non-wild type gene, a gene comprising a non-wild type reading frame, a gene comprising one or more mutations, or abnormal processing upon transcription of a gene. In some embodiments, the kits described herein are used in detection of a modified nucleic acid sequence associated with a disease or disorder associated gene. Also, by way of non-limiting example, the kits described herein are compatible with detection of a nucleic acid sequence selected from a viral genome, a prokaryotic genome, or a eukaryotic genome.

In some embodiments, kits are used in diagnosis of a disease or disorder associated with a non-wild type gene, a gene comprising a non-wild type reading frame; a gene comprising one or more mutations, or abnormal processing upon transcription of a gene. In some embodiments, kits are compatible with methods of diagnosis as disclosed herein, wherein a kit further comprises a detectable label or a nucleic acid comprising a detectable label capable of hybridizing to a target nucleic acid. In some embodiments, hybridizing to a target nucleic acid results in modification of a detectable label, which in turn emits a detectable signal upon modification.

Also described herein are kits comprising: (a) a polypeptide, or a nucleic acid encoding the polypeptide, and an engineered guide nucleic acid, or a nucleic acid that encodes the engineered guide nucleic acid; (b) a polypeptide, or a nucleic acid encoding the polypeptide, and an engineered guide nucleic acid comprising a crRNA; (c) an mRNA encoding a polypeptide, and an engineered guide nucleic acid; (d) one or more recombinant expression vectors comprising: i) a nucleic acid encoding a polypeptide; and ii) an engineered guide nucleic acid; or (e) one or more recombinant expression vectors comprising: i) a nucleic acid encoding a polypeptide; and ii) an engineered guide nucleic acid comprising a crRNA; wherein the polypeptide comprises an amino acid sequence that is at least 85% identical to any one of the sequences set forth in TABLE 1 or TABLE 1.2. In some embodiments, the components of the kit are in same container. In some embodiments, the components of the kit are in separate containers.

### Kit Components

In general, kit components comprise structural components as well as sample components, including compositions and systems described herein. Often, kits comprise one or more containers compatible for containing the samples, compositions, and systems described herein. In some embodiments, components of the samples, compositions, and systems are contained in the same container or in separate containers. In some embodiments, a container is a syringe, test wells, bottles, chambers, channels, vials, or test tubes. In one embodiment, the containers are formed from a variety of materials such as glass, plastic, or polymers. The system or systems described herein contain packaging materials. Examples of packaging materials include, but are not limited to, pouches, blister packs, bottles, tubes, bags, containers, bottles, and any packaging material suitable for intended mode of use.

In some embodiments, a kit comprises components, compositions, systems, and/or reagents for performing any methods disclosed herein. In some embodiments, a kit comprises components, compositions, and/or reagents for performing an assay disclosed herein. In some embodiments, a kit comprises other therapeutic agents, carriers, buffers, containers, devices for administration, and the like. In some embodiments, kits described herein comprise a solid support. In some embodiments, an RNP or effector protein is attached to a solid support. For example, in some embodiments, the solid support is an electrode or a bead. In some embodiments, the bead is a magnetic bead. Upon cleavage, the RNP is liberated from the solid support and interacts with other mixtures. For example, upon cleavage of the nucleic acid of the RNP, the effector protein of the RNP flows through a chamber into a mixture comprising a substrate. When the effector protein meets the substrate, a reaction occurs, such as a colorimetric reaction, which is then detected. As another example, the protein is an enzyme substrate, and upon cleavage of the nucleic acid of the enzyme substrate-nucleic acid, the enzyme flows through a chamber into a mixture comprising the enzyme. When the enzyme substrate meets the enzyme, a reaction occurs, such as a calorimetric reaction, which is then detected.

In some embodiments, the kit comprises labels and/or instructions for modifying, and/or detecting target nucleic acid. In some embodiments, the kit comprises labels and/or instructions for use. In some embodiments, labeling and/or instructions includes, for example, information concerning the amount, frequency and method of introduction and/or administration of the compositions, systems, and/or nucleic acid constructs described herein. In some embodiments, a label is on a container when letters, numbers or other characters forming the label are attached, molded, or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein. After packaging the formed product and wrapping or boxing to maintain a sterile barrier, in some embodiments, the product is terminally sterilized by heat sterilization, gas sterilization, gamma irradiation, or by electron beam sterilization. Alternatively, in some embodiments, the product is prepared and packaged by aseptic processing.

In some embodiments, the instructions for practicing the methods are recorded on a suitable recording medium. In some embodiments, the instructions are printed on a substrate, such as paper or plastic, etc. In some embodiments, the instructions are present in the kits as a package insert, in the labeling of the container of the kit or components thereof (i.e., associated with the packaging or subpackaging) etc. In some embodiments, the instructions are present as an electronic storage data file present on a suitable computer readable storage medium. e.g., CD-ROM, diskette, flash drive, etc. In some embodiments, the actual instructions are not present in the kit, but means for obtaining the instructions from a remote source (e.g., via the Internet), are provided. In some embodiments, the kit includes a web address where the instructions are viewed and/or from which the instructions are downloaded.

Also described herein are containers comprising: (a) a polypeptide, or a nucleic acid encoding the polypeptide, and an engineered guide nucleic acid, or a nucleic acid that encodes the engineered guide nucleic acid; (b) a polypeptide, or a nucleic acid encoding the polypeptide, and an engineered guide nucleic acid comprising a crRNA; (c) an mRNA encoding a polypeptide, and an engineered guide nucleic acid; (d) one or more recombinant expression vectors comprising: i) a nucleic acid encoding a polypeptide; and ii) an engineered guide nucleic acid; or (e) one or more recombinant expression vectors comprising: i) a nucleic acid encoding a polypeptide; and ii) an engineered guide nucleic acid comprising a crRNA; wherein the polypeptide comprises an amino acid sequence that is at least 85% identical to any one of the sequences set forth in TABLE 1 or TABLE 1.2. In some embodiments, the container is a syringe.

### XIII. Methods and Formulations for Introducing System Components and Compositions into a Target Cell

Disclosed herein, in some aspects, are systems and methods for introducing systems and components of such systems into a target cell. In some embodiments, the systems comprise, as described herein, one or more components having any one of the polypeptides (e.g., effector proteins, effector partners, fusion partners, fusion proteins, or combination thereof) or a nucleic acid comprising a nucleotide sequence encoding same. In some embodiments, such systems comprise, as described herein, one or more components having a guide nucleic acid or a nucleic acid comprising a nucleotide sequence encoding same. In some embodiments, systems comprise one or more components having a guide nucleic acid and an additional nucleic acid. In some embodiments, systems and components thereof are used to introduce the polypeptides, guide nucleic acids, or combinations thereof into a target cell. In some embodiments, the methods are used for modifying or editing a target nucleic acid. In some embodiments, systems comprise the polypeptide, one or more guide nucleic acids, and a reagent for facilitating the introduction of the polypeptide and the one or more guide nucleic acids. In some embodiments, system components for the methods comprise a solution, a buffer, a reagent for facilitating the introduction of the polypeptide and the one or more guide nucleic acids, or combinations thereof. In some embodiments, a guide nucleic acid (or a nucleic acid comprising a nucleotide sequence encoding same) and/or a polypeptide (e.g., effector protein, effector partner, fusion partner, fusion protein, or combination thereof) (or a nucleic acid comprising a nucleotide sequence encoding same) described herein are introduced into a host cell by any of a variety of well-known methods. As a non-limiting example, in some embodiments, the guide nucleic acid and/or polypeptide are combined with a lipid. As another non-limiting example, in some embodiments, the guide nucleic acid and/or polypeptide are combined with a particle or formulated into a particle.

### Methods for Introducing System Components and Compositions to a Host

Described herein are methods of introducing various components described herein to a host. A host may be any suitable host. In some embodiments, a host comprises a host cell. When described herein, a host cell comprises an in vivo or in vitro eukaryotic cell, a prokaryotic cell (e.g., bacterial or archaeal cell), or a cell from a multicellular organism (e.g., a cell line) cultured as a unicellular entity. In some embodiments, eukaryotic or prokaryotic cells are, or have been, used as recipients for methods of introduction described herein. In some embodiments, eukaryotic or prokaryotic cells comprise the progeny of the original cell which has been transformed by the methods of introduction described herein. It is understood that the progeny of a single cell is not necessarily be completely identical in morphology or in genomic or total DNA complement as the original parent, due to natural, accidental, or deliberate mutation. In some embodiments, a host cell comprises a recombinant host cell or a genetically modified host cell, if a heterologous nucleic acid, e.g., an expression vector, has been introduced into the cell.

Methods of introducing a nucleic acid and/or protein into a host cell are known in the art, and any convenient method may be used to introduce a subject nucleic acid (e.g., an expression construct/vector) into a target cell (e.g., a human cell). Suitable methods include, e.g., viral infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, and nanoparticle-mediated nucleic acid delivery (see, e.g., Panyam et al. Adv Drug Deliv Rev. 2012 Sep. 13. pii: 50169-409X(12)00283-9. doi: 10.1016/j.addr.2012.09.023). In some embodiments, the nucleic acid and/or protein(s) are introduced into a disease cell comprised in a pharmaceutical composition comprising the guide nucleic acid, the polypeptide, a pharmaceutically acceptable excipient, or combinations thereof.

In some embodiments, molecules of interest, such as nucleic acids of interest, are introduced to a host. In some embodiments, polypeptides are introduced to a host. In some embodiments, vectors, such as lipid particles and/or viral vectors are introduced to a host. In some embodiments, introduction is for contact with a host or for assimilation into the host, for example, introduction into a host cell.

In some embodiments, described herein are methods of introducing one or more nucleic acids, such as a nucleic acid encoding polypeptide, a nucleic acid that, when transcribed, produces an engineered guide nucleic acid, and/or a donor nucleic acid, or combinations thereof, into a host cell. Any suitable method may be used to introduce a nucleic acid into a cell. Suitable methods include, for example, viral infection, transfection, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct microinjection, and nanoparticle-mediated nucleic acid delivery. Further methods are described throughout.

In some embodiments, introducing one or more nucleic acids into a host cell occurs in any culture media and under any culture conditions that promote the survival of the cells. In some embodiments, introducing one or more nucleic acids into a host cell is carried out in vivo or ex vivo. In some embodiments, introducing one or more nucleic acids into a host cell is carried out in vitro.

In some embodiments, polypeptides (e.g., effector proteins, effector partners, fusion partners, fusion proteins, or combinations thereof) are provided as RNA. In some embodiments, the RNA is provided by direct chemical synthesis or is transcribed in vitro from a DNA (e.g., encoding the polypeptide). Once synthesized, the RNA is introduced into a cell by way of any suitable technique for introducing nucleic acids into cells (e.g., microinjection, electroporation, transfection, etc.). In some embodiments, introduction of one or more nucleic acid is through the use of a vector and/or a vector system, accordingly, in some embodiments, compositions and system described herein comprise a vector and/or a vector system.

In some embodiments, vectors are introduced directly to a host. In some embodiments, host cells are contacted with one or more vectors as described herein, and in some embodiments, said vectors are taken up by the cells. Methods for contacting cells with vectors include but are not limited to electroporation, calcium chloride transfection, microinjection, lipofection, micro-injection, contact with the cell or particle that comprises a molecule of interest, or a package of cells or particles that comprise molecules of interest.

In some embodiments, components described herein are introduced directly to a host. For example, in some embodiments, an engineered guide nucleic acid is introduced to a host, specifically introduced into a host cell. Methods of introducing nucleic acids, such as RNA into cells include, but are not limited to direct injection, transfection, or any other method used for the introduction of nucleic acids.

In some embodiments, polypeptides (e.g., effector proteins, effector partners, fusion partners, fusion proteins, or combinations thereof) described herein are introduced directly to a host. In some embodiments, polypeptides described herein are modified to promote introduction to a host. For example, in some embodiments, polypeptides described herein are modified to increase the solubility of the polypeptide. In some embodiments, the polypeptide is optionally fused to a polypeptide domain that increases solubility. In some embodiments, the domain is linked to the polypeptide through a defined protease cleavage site, such as TEV sequence which is cleaved by TEV protease. In some embodiments, the linker comprises one or more flexible sequences, e.g., from 1 to 10 glycine residues. In some embodiments, the cleavage of the polypeptide is performed in a buffer that maintains solubility of the product, e.g., in the presence of from 0.5 to 2 M urea, or in the presence of polypeptides and/or polynucleotides that increase solubility. Domains of interest include endosomolytic domains, e.g., influenza HA domain; and other polypeptides that aid in production, e.g., IF2 domain, GST domain, and GRPE domain. In another example, the polypeptide is modified to improve stability. For example, the polypeptides is PEGylated, where the polyethyleneoxy group provides for enhanced lifetime in the blood stream. In some embodiments, polypeptides are modified to promote uptake by a host, such as a host cell. For example, a polypeptide described herein is fused to a polypeptide permeant domain to promote uptake by a host cell. Any suitable permeant domains may be used in the non-integrating polypeptides of the present disclosure, including peptides, peptidomimetics, and non-peptide carriers. Examples include penetratin, a permeant peptide that is derived from the third alpha helix of Drosophila melanogaster transcription factor Antennapaedia; the HIV-1 tat basic region amino acid sequence, e.g., amino acids 49-57 of a naturally-occurring tat protein; and poly-arginine motifs, for example, the region of amino acids 34-56 of HIV-1 rev protein, nonaarginine, and octa-arginine. In some embodiments, the site at which the fusion is made is selected in order to optimize the biological activity, secretion or binding characteristics of the polypeptide. In some embodiments, the optimal site is determined by suitable methods.

### Formulations for Introducing System Components and Compositions to a Host

Described herein are formulations of introducing compositions or components of a system described herein to a host. In some embodiments, such formulations, systems and compositions described herein comprise polypeptides (e.g., effector proteins, effector partners, fusion partners, fusion proteins, or combinations thereof) and a carrier (e.g., excipient, diluent, vehicle, or filling agent). In some embodiments of the present disclosure, the polypeptides are provided in a pharmaceutical composition comprising the polypeptides and any pharmaceutically acceptable excipient, carrier, or diluent.

### XIV. Methods of Modifying a Nucleic Acid

Provided herein are compositions, methods, and systems for modifying (e.g., editing, cleaving) a nucleic acid (e.g., target nucleic acid or non-target nucleic acid). In general, modifying refers to changing the physical composition of a nucleic acid. However, compositions, methods, and systems disclosed herein modify nucleic acids, such as making epigenetic modifications of nucleic acids, which does not change the nucleotide sequence of the nucleic acids per se. In some embodiments, polypeptides (e.g., effector proteins, effector partners, fusion partners, fusion proteins, or combinations thereof), compositions and systems described herein are used for modifying a nucleic acid (e.g., target nucleic acid or non-target nucleic acid). In some embodiments, nucleic acid modifications described herein are initiated by the recognition of and/or cleaving of a nucleic acid (e.g., target nucleic acid or non-target nucleic acid) by an effector protein. In some embodiments, modifications of a nucleic acid results in certain measurable features, such as a detectable signal, silencing of gene expression, RNA degradation, and/or alternative splicing of a nucleic acid. In some embodiments, effector proteins described herein exhibit nucleic acid cleavage activity, such as cis cleavage activity, trans cleavage activity, nicking activity, and/or nuclease activity. In some embodiments, a catalytically inactive variant of an effector protein is fused to a fusion partner, wherein a catalytically inactive variant and a guide nucleic acid function to recruit the fusion partner to a target nucleic acid where the fusion partner modifies the target nucleic acid or expression thereof. In some embodiments, fusion partner activity described herein results in other nucleic acid modifications, such as deamination or demethylation. In some embodiments, modifying a nucleic acid comprises one or more of: methylating, demethylating, deaminating, or oxidizing one or more nucleotides of the nucleic acid (e.g., target nucleic acid). In some embodiments, modifying a nucleic acid comprises modifying a target nucleic acid sequence or at least one nucleotide of a target nucleic acid.

In some embodiments, compositions, methods, and systems described herein modify a nucleic acid (e.g., target nucleic acid) associated with a coding portion of a gene, a non-coding portion of a gene, or a combination thereof. In some embodiments, modifying at least one nucleic acid associated with a gene using the compositions, methods or systems described herein modifies (e.g., reduce or increase) expression of one or more proteins and/or genes. In some embodiments, the compositions, methods or systems reduce expression of one or more proteins and/or genes by at least 10%, at least 20%, at least 30%, at least 40%, at least 50%, at least 60%, at least 70%, at least 80%, at least 90%, or at least 95%. In some embodiments, the compositions, methods or systems remove all expression of a proteins and/or gene. In some embodiments, the compositions, methods or systems increase expression of one or more proteins and/or genes by at least 10%, at least 20%, at least 30%, at least 40%, at least 50%, at least 60%, at least 70%, at least 80%, at least 90%, or at least 100%.

In some embodiments, the compositions, methods or systems comprise a nucleic acid expression vector, or use thereof, to introduce polypeptides (e.g., effector proteins, effector partners, fusion partners, fusion proteins, or combinations thereof), guide nucleic acid, or any combination thereof, to a cell. In some embodiments, the nucleic acid expression vector is a viral vector. Viral vectors include, but are not limited to, retroviruses, adenoviruses, adeno-associated viruses, and herpes simplex viruses. In some embodiments, the viral vector is a replication-defective viral vector, comprising an insertion of a therapeutic gene inserted in genes essential to the lytic cycle, preventing the virus from replicating and exerting cytotoxic effects. In some embodiments, the viral vector is an adeno associated viral (AAV) vector. In some embodiments, the nucleic acid expression vector is a non-viral vector. In some embodiments, compositions and methods comprise a lipid, polymer, nanoparticle, or a combination thereof, or use thereof, to introduce the polypeptide (e.g., a Cas protein), guide nucleic acid, donor template or any combination thereof to a cell. Non-limiting examples of lipids and polymers are cationic polymers, cationic lipids, or bio-responsive polymers. In some embodiments, the bio-responsive polymer exploits chemical-physical properties of the endosomal environment (e.g., pH) to preferentially release the genetic material in the intracellular space.

In some embodiments, methods of modifying comprise contacting a target nucleic acid with one or more components, compositions or systems described herein. In some embodiments, a method of modifying comprises contacting a target nucleic acid with at least one of: a) one or more polypeptides (e.g., effector proteins, effector partners, fusion partners, fusion proteins, or combinations thereof), or one or more nucleic acids encoding the one or more polypeptides; or b) one or more guide nucleic acids, or one or more nucleic acids encoding one or more guide nucleic acids. In some embodiments, a method of modifying comprises contacting a target nucleic acid with a system described herein wherein the system comprises components comprising at least one of: a) one or more polypeptides (e.g., effector proteins, effector partners, fusion partners, fusion proteins, or combinations thereof), or one or more nucleic acids encoding the one or more polypeptides; or b) one or more guide nucleic acids, or one or more nucleic acids encoding one or more guide nucleic acids. In some embodiments, a method of modifying comprises contacting a target nucleic acid with a composition described herein comprising at least one of: a) one or more polypeptides (e.g., effector proteins, effector partners, fusion partners, fusion proteins, or combinations thereof), or one or more nucleic acids encoding the one or more polypeptides; or b) one or more guide nucleic acids, or one or more nucleic acids encoding one or more guide nucleic acids; in a composition. In some embodiments, a method of modifying as described herein produces a modified target nucleic acid. In some embodiments, a method of modifying as described herein comprises modifying at least one nucleotide of a target nucleic acid.

In some embodiments, nucleic acid modifications are initiated by the recognition in/or cleaving of a nucleic acid (e.g., target nucleic acid or non-target nucleic acid) by an effector protein, which, in some embodiments, results in certain measurable features, such as a detectable signal, silencing of gene expression, RNA degradation, and/or alternative splicing of a nucleic acid (e.g., target nucleic acid or non-target nucleic acid). In some embodiments, degradation or alternative splicing of a nucleic acid as described herein comprises degradation or alternative splicing of at least one nucleotide of the nucleic acid. In some embodiments, alternative splicing of a nucleic acid comprises removal or degradation of a sequence of interest, a non-coding region, an intron, an intron fragment, or any combination thereof. In some embodiments, alternative splicing of a nucleic acid comprises rejoining of a sequence of interest, a coding region, an exon, an exon fragment, or any combination thereof.

Also described herein are methods of modifying a target nucleic acid, the methods comprising contacting the target nucleic acid any one of the systems described herein, or the pharmaceutical compositions described herein, thereby producing a modified target nucleic acid. In some embodiments, modifying the target nucleic acid comprises splicing, silencing, or degradation of the target nucleic acid or a target sequence in the target nucleic acid. In some embodiments, splicing the target nucleic acid comprises removal or degradation of a sequence of interest, a non-coding region, an intron, an intron fragment, or any combinations thereof. In some embodiments, splicing the target nucleic acid comprises rejoining of a sequence of interest, a coding region, an exon, an exon fragment, or any combinations thereof. In some embodiments, the method is performed in in vitro. In some embodiments, the target nucleic acid is associated with any one of the genes recited in TABLE 4 or TABLE 4.1. In some embodiments, the target nucleic acid comprises a mutation associated with a disease or disorder. In some embodiments, the target nucleic acid comprises one or more mutations. In some embodiments, the one or more mutations comprise a point mutation, a single nucleotide polymorphism (SNP), a missense mutation, a nonsense mutation, a transcription-associated mutation, or any combination thereof. In some embodiments, the disease or disorder is any one of the diseases or disorders recited in TABLE 5 or TABLE 5.1. In some embodiments, the modified target nucleic acid no longer comprises a mutation associated with a disease or disorder as compared to an unmodified target nucleic acid. In some embodiments, the modified target nucleic acid produces a polypeptide having new activity as compared to an unmodified target nucleic acid, or alters expression of an endogenous polypeptide as compared to an unmodified target nucleic acid.

In some embodiments, nucleic acid modifications described herein comprise introducing a mutation (e.g., point mutation) in a target nucleic acid sequence relative to a corresponding wildtype nucleotide sequence. In some embodiments, target nucleic acid modifications described herein remove or correct a mutation associated with a disease or disorder recited in TABLE 5 or TABLE 5.1. In some embodiments, a modified target nucleic acid no longer comprise a mutation associated with a disease or disorder described herein. In some embodiments, a modified target nucleic acid encodes a protein having new activity as compared to an unmodified target nucleic acid. In some embodiments, a modified target nucleic acid modifies the expression of an endogenous protein as compared to an unmodified target nucleic acid. In some embodiments, a modified nucleic acid no longer comprises a mutation associated with mis-expression or mutation of a gene associated with a disease or disorder described herein. In some embodiments, modifying a target nucleic acid sequence removes/corrects point mutations, deletions, insertions, or null mutations in a target nucleic acid. In some embodiments, modifying a target nucleic acid sequence is used to generate a target protein capable of a desired protein activity. In some embodiments, methods of the disclosure are targeted to modifying a target nucleic acid associated with any locus in a genome of a cell.

In some embodiments, modifying comprises single stranded cleavage, double stranded cleavage, epigenetic modification (e.g., methylation, demethylation, acetylation, or deacetylation), or a combination thereof. In some embodiments, cleavage (single-stranded or double-stranded) is site-specific, meaning cleavage occurs at a specific site in the target nucleic acid, often within the region of the target nucleic acid that hybridizes with the guide nucleic acid spacer sequence. In some embodiments, the polypeptides (e.g., effector proteins, effector partners, fusion partners, fusion proteins, or combinations thereof) introduce a single-stranded break in a target nucleic acid to produce a cleaved nucleic acid. In some embodiments, the polypeptide introduces a break in a single stranded RNA (ssRNA). In some embodiments, the polypeptide is coupled to a guide nucleic acid that targets a particular region of interest in the ssRNA. In some embodiments, the target nucleic acid is modified wherein modifying comprises deleting one or more nucleotides of the target nucleic acid, inserting one or more nucleotides into the target nucleic acid, substituting one or more nucleotides of the target nucleic acid with one or more alternative nucleotides, or combinations thereof. In some embodiments, modifying the target nucleic acid comprises modifying a nucleobase of at least one nucleotide. In some embodiments, modifying the target nucleic acid reverses a mutation or the expression of a mutation. In some embodiments, modifying the target nucleic acid involves substitution, insertion, or deletion. In some embodiments, modifying the target nucleic acid reverses an indel or the expression of an indel, sometimes referred to as an insertion-deletion or indel mutation, which is a type of genetic mutation that results from the insertion and/or deletion of one or more nucleotide(s) in a nucleic acid (e.g., RNA). In some embodiments, an indel varies in length (e.g., 1 to 1,000 nucleotides in length) and be detected using methods well known in the art, including sequencing. In some embodiments, an indel mutation is associated to a frameshift mutation. Indel percentage is the percentage of sequencing reads that show at least one nucleotide has been mutation that results from the insertion and/or deletion of nucleotides regardless of the size of insertion or deletion, or number of nucleotides mutated. For example, if there is at least one nucleotide deletion detected in a given target nucleic acid, it counts towards the percent indel value. As another example, if one copy of the target nucleic acid has one nucleotide deleted, and another copy of the target nucleic acid has 10 nucleotides deleted, they are counted the same. This number reflects the percentage of target nucleic acids that, in some embodiments, is modified by a given polypeptide.

In some embodiments, methods of modifying described herein cleave a target nucleic acid at one or more locations to generate a cleaved target nucleic acid. In some embodiments, the cleaved target nucleic acid undergoes recombination. In some embodiments, cleavage in the target nucleic acid is repaired.

In some embodiments, wherein the compositions, systems, and methods of the present disclosure restore a wild-type reading frame. In some embodiments, a wild-type reading frame comprises a reading frame that produces at least a partially, or fully, functional protein. In some embodiments, a non-wild-type reading frame comprises a reading frame that produces a non-functional or partially non-functional protein.

Accordingly, in some embodiments, compositions, systems, and methods described herein modify 1 to 1,000 nucleotides or any integer in between, in a target nucleic acid. In some embodiments, 1 to 1,000, 2 to 900, 3 to 800, 4 to 700, 5 to 600, 6 to 500, 7 to 400, 8 to 300, 9 to 200, or 10 to 100 nucleotides, or any integer in between, are modified by the compositions, systems, and methods described herein. In some embodiments, 1, 2, 3, 4, 5, 6, 7, 8, 9, 10, or more nucleotides are modified by the compositions, systems, and methods described herein. In some embodiments, 10, 20, 30, 40, 50, 60, 70, 80 90, 100 or more nucleotides, or any integer in between, are modified by the compositions, systems, and methods described herein. In some embodiments, 100, 200, 300, 400, 500, 600, 700, 800, 900 or more nucleotides, or any integer in between, are modified by the compositions, systems, and methods described herein.

In some embodiments, methods comprise use of two or more polypeptides (e.g., effector proteins, effector partners, fusion partners, fusion proteins, or combinations thereof). An illustrative method for introducing a break in a target nucleic acid comprises contacting the target nucleic acid with: (a) a first engineered guide nucleic acid comprising a region that binds to a first polypeptide described herein; and (b) a second engineered guide nucleic acid comprising a region that binds to a second polypeptide described herein, wherein the first engineered guide nucleic acid comprises an additional region that hybridizes to the target nucleic acid and wherein the second engineered guide nucleic acid comprises an additional region that hybridizes to the target nucleic acid. In some embodiments, the first and second polypeptide are identical. In some embodiments, the first and second polypeptide are not identical.

In some embodiments, modifying a target nucleic acid comprises modifying a nucleic acid associated with a genome. In some embodiments, modifying comprises modifying a nucleic acid associated with a genome, chromosome, plasmid, or other genetic material of a cell or organism. In some embodiments, the nucleic acid associated with a genome, chromosome, plasmid, or other genetic material of the cell or organism is modified in vivo. In some embodiments, the nucleic acid associated with a genome, chromosome, plasmid, or other genetic material of the cell or organism is modified in a cell. In some embodiments, the nucleic acid associated with a genome, chromosome, plasmid, or other genetic material of the cell or organism is modified in vitro. In some embodiments, modifying a nucleic acid (e.g., target nucleic acid or non-target nucleic acid) comprises modifying a nucleic acid generated by a genome, chromosome, plasmid, or other genetic material of a cell or organism. For example, in some embodiments, a plasmid is modified in vitro using a composition described herein and introduced into a cell or organism.

In some embodiments, modifying a nucleic acid (e.g., target nucleic acid or non-target nucleic acid) comprises deleting a sequence from a nucleic acid. For example, in some embodiments, a mutated sequence or a sequence associated with a disease is removed from a target nucleic acid. In some embodiments, modifying a target nucleic acid comprises replacing a sequence in a target nucleic acid with a second region or sequence. For example, in some embodiments, a mutated sequence or a sequence associated with a disease is replaced with a second region or sequence lacking the mutation or that is not associated with the disease. In some embodiments, modifying a target nucleic acid comprises deleting or replacing a sequence comprising markers associated with a disease or disorder. In some embodiments, modifying a target nucleic acid comprises introducing a sequence into a target nucleic acid. For example, in some embodiments, a beneficial sequence or a sequence that reduces or eliminates a disease is inserted into the target nucleic acid.

In some embodiments, methods comprise modifying a nucleic acid (e.g., target nucleic acid or non-target nucleic acid) with two or more polypeptides (e.g., effector proteins, effector partners, fusion partners, fusion proteins or combinations thereof). In some embodiments, modifying a target nucleic acid comprises introducing two or more single-stranded breaks in a target nucleic acid. In some embodiments, a break is introduced by contacting a target nucleic acid with an effector protein and a guide nucleic acid. In some embodiments, the guide nucleic acid binds to the effector protein and hybridizes to a region of the target nucleic acid, thereby recruits the effector protein to the region of the target nucleic acid. In some embodiments, binding of the effector protein to the guide nucleic acid and the region of the target nucleic acid activate the effector protein, and the activated effector protein introduces a break (e.g., a single stranded break) in the region of the target nucleic acid. In some embodiments, modifying a target nucleic acid comprises introducing a first break in a first region or sequence of the target nucleic acid and a second break in a second region or sequence of the target nucleic acid. For example, in some embodiments, modifying a target nucleic acid comprises contacting a target nucleic acid with a first guide nucleic acid that binds to a first effector protein and hybridizes to a first region or sequence of the target nucleic acid and a second guide nucleic acid that binds to a second effector protein or programmable nickase and hybridizes to a second region or sequence of the target nucleic acid. In some embodiments, the first effector protein introduces a first break in a first strand at the first region or sequence of the target nucleic acid, and the second effector protein introduces a second break in a second strand at the second region or sequence of the target nucleic acid. In some embodiments, a segment of the target nucleic acid between the first break and the second break is removed, thereby modifying the target nucleic acid.

In some embodiments, modifying of a target nucleic acid as described herein effects one or more mutations comprising splicing error mutations, frameshift mutations (e.g., 1+ or 2+ frameshift mutation), sequence deletion, sequence or exon skipping, sequence reframing, null mutations, or any combination thereof. In some embodiments, the splicing error can be a modifying that disrupts a splicing of a target nucleic acid or a nucleic acid associated with a target nucleic acid. In some embodiments, the frameshift mutation can be a modifying that alters the reading frame of a target nucleic acid relative to a target nucleic acid without the frameshift mutation. In some embodiments, the frameshift mutation can be a +2 frameshift mutation, wherein a reading frame is modified by 2 bases. In some embodiments, the frameshift mutation can be a +1 frameshift mutation, wherein a reading frame is modified by 1 base. In some embodiments, the frameshift mutation is a modifying that alters the number of bases in a target nucleic acid so that it is not divisible by three. In some embodiments, the frameshift mutation can be a modifying that is not a splicing error. In some embodiments, a sequence as described in reference to the nucleotide sequence deletion, sequence or exon skipping, and sequence reframing can be a RNA sequence, a modified RNA sequence, a mutated sequence, a wild-type sequence, a coding sequence, a non-coding sequence, an exonic sequence (exon), an intronic sequence (intron), or any combination thereof. In some embodiments, the nucleotide sequence deletion is a modifying where one or more sequences in a target nucleic acid are deleted relative to a target nucleic acid without the nucleotide sequence deletion. In some embodiments, the nucleotide sequence deletion can result in or effect a splicing error or a frameshift mutation. In some embodiments, a splicing error can result in sequence deletion. In some embodiments, the nucleotide sequence deletion results in or effects a splicing disruption. In some embodiments, the nucleotide sequence or exon skipping is a modifying where one or more sequences in a target nucleic acid are skipped during splicing of the target nucleic acid such that one or more sequences are deleted from the modified target nucleic acid relative to an unmodified target nucleic acid. In some embodiments, the nucleotide sequence or exon skipping can resolve a frameshift mutation. In some embodiments, the nucleotide sequence or exon skipping can result in or effect a splicing error. In some embodiments, the nucleotide sequence reframing is a modifying where one or more bases in a target are modified so that the reading frame of the nucleotide sequence is reframed relative to a target nucleic acid without the nucleotide sequence reframing. In some embodiments, the nucleotide sequence reframing can result in or effect a frameshift mutation. In some embodiments, the null mutation is a modifying where the modified target nucleic acid can no longer be translated into a functional protein.

In some embodiments, modifying of a target nucleic acid can be locus specific, wherein compositions, systems, and methods described herein can modify a target nucleic acid associated with one or more specific loci. Locus-specific target nucleic acids can be modified to effect one or more specific mutations comprising splicing error mutations, frameshift mutations, sequence deletion, sequence or exon skipping, sequence reframing, null mutation, or any combination thereof. For example, modifying of nucleic acids associated with a specific locus can affect any one of a splicing error, frameshift (e.g., 1+ or 2+ frameshift), sequence deletion, sequence or exon skipping, sequence reframing, null mutation, or any combination thereof. In some embodiments, modifying of a target nucleic acid can be locus specific, modification specific, or both. In some embodiments, modifying of a target nucleic acid can be locus specific, modification specific, or both, wherein compositions, systems, and methods described herein comprise polypeptides (e.g., effector proteins, effector partners, fusion partners, fusion proteins, or combinations thereof) described herein and a guide nucleic acid described herein.

In some embodiments, methods of modifying a target nucleic acid or modulating the expression of a target nucleic acid is performed in vivo. In some embodiments, methods of modifying a target nucleic acid or modulating the expression of a target nucleic acid is performed in vitro. For example, in some embodiments, a plasmid is modified in vitro using a composition described herein and introduced into a cell or organism. In some embodiments, methods of modifying a target nucleic acid or modulating the expression of a target nucleic acid is performed ex vivo. For example, in some embodiments, methods comprise obtaining a cell from a subject, modifying a target nucleic acid in the cell with methods described herein, and returning the cell to the subject.

In some embodiments, methods of modifying described herein comprise contacting a target nucleic acid with one or more components, compositions or systems described herein. In some embodiments, the one or more components, compositions or systems described herein comprise at least one of: a) one or more polypeptides (e.g., effector proteins, effector partners, fusion partners, fusion proteins, or combinations thereof), or one or more nucleic acids encoding the one or more polypeptides; and b) one or more guide nucleic acids, or one or more nucleic acids encoding the one or more guide nucleic acids. In some embodiments, the one or more polypeptides introduce a single-stranded break or a double-stranded break in the target nucleic acid. In some embodiments, methods of modifying described herein produce a modified target nucleic acid comprising an engineered nucleic acid sequence that expresses polypeptide having new activity as compared to an unmodified target nucleic acid, or alters expression of an endogenous polypeptide as compared to an unmodified target nucleic acid.

In some embodiments, methods of modifying described herein comprise the target nucleic acid comprises splicing, silencing, or degradation of the target nucleic acid or the target sequence in a target nucleic acid. In some embodiments, splicing the target nucleic acid comprises removal or degradation of a sequence of interest, a non-coding region, an intron, an intron fragment, or any combinations thereof. In some embodiments, splicing the target nucleic acid comprises rejoining of a sequence of interest, a coding region, an exon, an exon fragment, or any combinations thereof. In some embodiments, wherein the target nucleic acid is associated with any one of the nucleic acids recited in TABLE 4 and TABLE 4.1.

In some embodiments, methods of modifying described herein comprise using one or more guide nucleic acids or uses thereof, wherein the methods modify a target nucleic acid at a single location. In some embodiments, the methods comprise contacting an RNP comprising a polypeptides (e.g., effector proteins, effector partners, fusion partners, fusion proteins, or combinations thereof) and a guide nucleic acid to the target nucleic acid. In some embodiments, the methods introduce a mutation (e.g., point mutations, deletions) in the target nucleic acid relative to a corresponding wildtype nucleotide sequence. In some embodiments, the methods remove or correct a disease-causing mutation in a nucleic acid sequence to produce a corresponding wildtype nucleotide sequence. In some embodiments, the methods remove/correct point mutations, deletions, insertions, or null mutations in a target nucleic acid. In some embodiments, the methods introduce a single stranded cleavage, a nick, a deletion of one or two nucleotides, an insertion of one or two nucleotides, a substitution of one or two nucleotides, an epigenetic modification (e.g., methylation, demethylation, acetylation, or deacetylation), or a combination thereof to the target nucleic acid. In some embodiments, the methods comprise using an effector protein and two guide nucleic acids, wherein two RNPs cleave the target nucleic acid at the same location, wherein a first RNP comprises the effector protein and a first guide nucleic acid, and wherein a second RNP comprises the effector protein and a second guide nucleic acid. In some embodiments, methods comprising using two effector protein and two guide nucleic acids, wherein both RNPs cleave the target nucleic acid at the same location, wherein a first RNP comprises a first effector protein and a first target nucleic acid, and wherein a second RNP comprises a second effector protein and a second target nucleic acid.

In some embodiments, methods of modifying described herein comprise using one or more guide nucleic acids or uses thereof, wherein the methods modify a target nucleic acid at two different locations. In some embodiments, the methods introduce two cleavage sites in the target nucleic acid, wherein a first cleavage site and a second cleavage site comprise one or more nucleotides therebetween. In some embodiments, the methods cause deletion of the one or more nucleotides. In some embodiments, the deletion restores a wild-type reading frame. In some embodiments, the wild-type reading frame produces at least a partially functional protein. In some embodiments, the deletion causes a non-wild-type reading frame. In some embodiments, a non-wild-type reading frame produces a partially functional protein or non-functional protein. In some embodiments, the at least partially functional protein has at least 50%, at least 60%, at least 70%, at least 80%, at least 90%, at least 100%, at least 110%, at least 120%, at least 130%, at least 140%, at least 150%, at least 180%, at least 200%, at least 300%, at least 400% activity compared to a corresponding wildtype protein. In some embodiments, the methods comprise using an effector protein and two guide nucleic acids, wherein two RNPs cleave the target nucleic acid at different locations, wherein a first RNP comprises the effector protein and a first guide nucleic acid, and wherein a second RNP comprises the effector protein and a second guide nucleic acid. In some embodiments, methods comprising using two effector protein and two guide nucleic acids, wherein both RNPs cleave the target nucleic acid at the same location, wherein a first RNP comprises a first effector protein and a first target nucleic acid, and wherein a second RNP comprises a second effector protein and a second target nucleic acid.

### XV. Methods of Detecting a Target Nucleic Acid

Provided herein are methods of detecting target nucleic acids. In some embodiments, methods comprise detecting target nucleic acids with compositions or systems described herein. In some embodiments, methods comprise detecting a target nucleic acid in a sample, e.g., a cell lysate, a biological fluid, or environmental sample. In some embodiments, methods comprise detecting a target nucleic acid in a cell. In some embodiments, methods of detecting a target nucleic acid in a sample or cell comprises contacting the sample or cell with an effector protein or a multiprotein complex thereof, a guide nucleic acid, wherein at least a portion of the guide nucleic acid is complementary to at least a portion of the target nucleic acid, and a reporter nucleic acid that is cleaved in the presence of the effector protein, the guide nucleic acid, and the target nucleic acid, and detecting a signal produced by cleavage of the reporter nucleic acid, thereby detecting the target nucleic acid in the sample. In some embodiments, methods result in trans cleavage of the reporter nucleic acid. In some embodiments, methods result in cis cleavage of the reporter nucleic acid. In some embodiments, methods of detecting a target nucleic acid include a reporter nucleic acid comprising a detectable moiety that produces a detectable signal in the presence of the target nucleic acid, the effector protein, and the guide nucleic acid.

In some embodiments, the methods of detecting a target nucleic acid comprising: a) contacting the target nucleic acid with a composition comprising an effector protein as described herein, a guide nucleic acid as described herein, and a reporter nucleic acid that is cleaved in the presence of the effector protein, the guide nucleic acid, and the target nucleic acid; and b) detecting a signal produced by cleavage of the reporter nucleic acid, thereby detecting the target nucleic acid in the sample. In some embodiments, the methods result in trans cleavage of the reporter nucleic acid. In some embodiments, the methods result in cis cleavage of the reporter nucleic acid. In some embodiments, the reporter nucleic acid is a single stranded nucleic acid. In some embodiments, the reporter comprises a detection moiety. In some embodiments, the reporter nucleic acid is cleaved by the effector protein. In some embodiments, a cleaved reporter nucleic acid generates a detectable product or a first detectable signal. In some embodiments, the first detectable signal is a change in color. In some embodiments, the change is color is measured indicating presence of the target nucleic acid. In some embodiments, the first detectable signal is measured on a support medium.

In some embodiments, methods of detecting described herein comprise contacting a target nucleic acid, a cell comprising the target nucleic acid, or a sample comprising a target nucleic acid with an effector protein that comprises an amino acid sequence that is at least is at least 70%, at least 75%, at least 80%, at least 85%, at least 90%, at least 92%, at least 95%, at least 97%, at least 98%, at least 99%, or 100% identical to any one of the amino acid sequences set forth in TABLE 1 or TABLE 1.2. In some embodiments, the amino acid sequence of the effector protein is at least 70%, at least 75%, at least 80%, at least 85%, at least 90%, at least 92%, at least 95%, at least 97%, at least 98%, at least 99%, or 100% identical to any one of the amino acid sequences set forth in TABLE 1 or TABLE 1.2. In some embodiments, the effector protein comprising an amino acid sequence that is at least 90% identical to a sequence selected from any one of the amino acid sequences set forth in TABLE 1 or TABLE 1.2.

In some embodiments, methods comprise contacting the sample to a complex comprising a guide nucleic acid comprising a segment that is reverse complementary to a segment of the target nucleic acid and an effector protein that exhibits sequence independent cleavage upon forming a complex comprising the segment of the guide nucleic acid binding to the segment of the target nucleic acid; and assaying for a signal indicating cleavage of at least some protein-nucleic acids of a population of protein-nucleic acids, wherein the signal indicates a presence of the target nucleic acid in the sample and wherein absence of the signal indicates an absence of the target nucleic acid in the sample.

In some embodiments, methods comprise contacting the sample comprising the target nucleic acid with a guide nucleic acid targeting a target nucleic acid segment, an effector protein that is activated when complexed with the guide nucleic acid and the target nucleic acid segment, a single stranded nucleic acid of a reporter comprising a detection moiety, wherein the nucleic acid of a reporter is cleaved by the activated effector protein, thereby generating a first detectable signal, cleaving the single stranded nucleic acid of a reporter using the effector protein that cleaves as measured by a change in color, and measuring the first detectable signal on the support medium.

In some embodiments, methods comprise contacting the sample or cell with an effector protein or a multiprotein complex thereof and a guide nucleic acid at a temperature of at least about 25° C., at least about 30° C., at least about 35° C., at least about 37° C., at least about 40° C., at least about 50° C., at least about 65° C., at least about 70° C., or at least about 75° C. In some embodiments, the temperature is not greater than 80° C. In some embodiments, the temperature is about 25° C., about 30° C., about 35° C., at least about 37° C., about 40° C., about 45° C., about 50° C., about 55° C., about 60° C., about 65° C., about 70° C., about 75° C., about 80° C., about 85° C., or about 90° C. In some embodiments, the temperature is about 25° C. to about 45° C., about 35° C. to about 55° C., about 37° C. to about 60° C., or about 55° C. to about 65° C. In some embodiments, the temperature is about 37° C. to about 45° C., about 37° C. to about 50° C., about 37° C. to about 55° C., about 37° C. to about 60° C., or about 37° C. to about 65° C.

In some embodiments, methods comprise contacting the sample or cell with an effector protein or a multiprotein complex thereof and a guide nucleic acid in the presence of salts (e.g., compositions comprising salts). In some embodiments, the method comprises a solution, wherein the solution comprises one or more salt. Accordingly, in some embodiments, the salt comprises one or more salt selected from a magnesium salt, a zinc salt, a potassium salt, a calcium salt, and a sodium salt. In some embodiments, the salt is a combination of two or more salts. For example, in some embodiments, the salt is a combination of two or more salts selected from a magnesium salt, a zinc salt, a potassium salt, a calcium salt and a sodium salt. In some embodiments, the salt is magnesium acetate. In some embodiments, the salt is magnesium chloride. In some embodiments, the salt is potassium acetate. In some embodiments, the salt is potassium nitrate. In some embodiments, the salt is zinc chloride. In embodiments, the salt is sodium chloride. In some embodiments, the salt is potassium chloride. In some embodiments, the concentration of the one or more salt in the solution is about 0.001 mM to about 500 mM. In some embodiments, the concentration of the salt is about 0.001 mM to about 400 mM. In some embodiments, the concentration of the salt is about 0.001 mM to about 300 mM. In some embodiments, the concentration of the salt is about 0.001 mM to about 200 mM. In some embodiments, the concentration of the salt is about 0.001 mM to about 100 mM. In some embodiments, the concentration of the salt is about 0.001 mM to about 10 mM. In some embodiments, the concentration of the salt is about 0.01 mM to about 500 mM. In some embodiments, the concentration of the salt is about 0.01 mM to about 400 mM. In some embodiments, the concentration of the salt is about 0.01 mM to about 300 mM. In some embodiments, the concentration of the salt is about 0.01 mM to about 200 mM. In some embodiments, the concentration of the salt is about 0.01 mM to about 100 mM. In some embodiments, the concentration of the salt is about 0.01 mM to about 10 mM. In some embodiments, the concentration of the salt is about 0.1 mM to about 500 mM. In some embodiments, the concentration of the salt is about 0.1 mM to about 400 mM. In some embodiments, the concentration of the salt is about 0.1 mM to about 300 mM. In some embodiments, the concentration of the salt is about 0.1 mM to about 200 mM. In some embodiments, the concentration of the salt is about 0.1 mM to about 100 mM. In some embodiments, the concentration of the salt is about 0.1 mM to about 10 mM. In some embodiments, the concentration of the salt is about 1 mM to about 500 mM. In some embodiments, the concentration of the salt is about 1 mM to about 400 mM. In some embodiments, the concentration of the salt is about 1 mM to about 300 mM. In some embodiments, the concentration of the salt is about 1 mM to about 200 mM. In some embodiments, the concentration of the salt is about 1 mM to about 100 mM. In some embodiments, the concentration of the salt is about 1 mM to about 10 mM. In some embodiments, the concentration of the salt is about 10 mM to about 500 mM. In some embodiments, the concentration of the salt is about 10 mM to about 400 mM. In some embodiments, the concentration of the salt is about 10 mM to about 300 mM. In some embodiments, the concentration of the salt is about 10 mM to about 200 mM. In some embodiments, the concentration of the salt is about 10 mM to about 100 mM. In some embodiments, the concentration of the salt is about 100 mM to about 500 mM. In some embodiments, the concentration of the salt is about 100 mM to about 400 mM. In some embodiments, the concentration of the salt is about 100 mM to about 300 mM. In some embodiments, the concentration of the salt is about 100 mM to about 200 mM. In some embodiments, the salt is potassium acetate and the concentration of salt in the solution is about 100 mM. In some embodiments, the salt is potassium acetate or sodium chloride and the concentration of salt in the solution is about 200 mM. In some embodiments, the salt is potassium acetate or sodium chloride and the salt of potassium in the solution is about 100 mM to about 200 mM.

In some embodiments, methods of detecting a target nucleic acid by a cleavage assay. In some embodiments, the target nucleic acid is a single-stranded target nucleic acid. In some embodiments, the cleavage assay comprises: a) contacting the target nucleic acid with a composition comprising an effector protein as described; and b) cleaving the target nucleic acid. In some embodiments, the cleavage assay comprises an assay designed to visualize, quantitate or identify cleavage of a nucleic acid. In some embodiments, the method is an in vitro trans cleavage assay. In some embodiments, a cleavage activity is a trans cleavage activity. In some embodiments, the method is an in vitro cis cleavage assay. In some embodiments, a cleavage activity is a cis cleavage activity. In some embodiments, the cleavage assay follows a procedure comprising: (i) providing a composition comprising an equimolar amounts of an effector protein as described herein, and a guide nucleic acid described herein, under conditions to form an RNP complex; (ii) adding a plasmid comprising a target nucleic acid, wherein the target nucleic acid is a linear dsRNA, wherein the target nucleic acid is single-stranded RNA (ssRNA), double stranded RNA (dsRNA), linear RNA, circular RNA, coding RNA, non-coding RNA, or combinations thereof; (iii) incubating the mixture under conditions to enable cleavage of the plasmid; (iv) quenching the reaction with EDTA and a protease; and (v) analyzing the reaction products (e.g., viewing the cleaved and uncleaved linear dsDNA with gel electrophoresis).

In some embodiments, the methods of detecting a target nucleic acid comprises: a) contacting the target nucleic acid with a composition comprising an effector protein as described herein, a guide nucleic acid as described herein, the target nucleic acid, and the non-target nucleic acid; b) cleaving the non-target sequence in a non-target nucleic acid with the effector protein in response to the formation of a complex comprising the effector protein, the guide nucleic acid, the target sequence in a target nucleic acid, thereby producing a detectable product; and c) detecting the detectable product produced by cleavage of the non-target nucleic acid, thereby detecting the presence of the target nucleic acid in the sample. In some embodiments, the methods result in trans cleavage of the non-target nucleic acid. In some embodiments, the target nucleic acid is amplified DNA, DNA synthesized from a single-stranded RNA template, double-stranded DNA, single-stranded DNA, cDNA, or combinations thereof. In some embodiments, the non-target nucleic acid is part of a reporter. In some embodiments, the effector protein is capable of both hybridizing to the target nucleic acid and cleaving the non-target sequence. In some embodiments, the amplified DNA, DNA synthesized from a single-stranded RNA template, or cDNA is double-stranded DNA, single-stranded DNA, or combinations thereof.

Also described herein are methods of detecting a presence of a target nucleic acid in a sample. In some embodiments, such a method comprises: (a) contacting the sample with any one of the systems described herein; (b) cleaving a reporter with the polypeptide in response to formation of a complex comprising the polypeptide, an engineered guide nucleic acid, and a target sequence in a target nucleic acid, thereby producing a detectable product; and (c) detecting the detectable product, thereby detecting the presence of the target nucleic acid in the sample. In some embodiments, the target nucleic acid is double-stranded RNA, single-stranded RNA, linear RNA, circular RNA, coding RNA, non-coding RNA, or combinations thereof.

Also provided herein are methods of detecting a presence of a target nucleic acid in a sample, the method comprising: (a) contacting the sample with any one of the systems described herein; (b) cleaving a non-target sequence in a non-target nucleic acid with the polypeptide in response to formation of a complex comprising the polypeptide, an engineered guide nucleic acid, and a target sequence in a target nucleic acid, thereby producing a detectable product; and (c) detecting the detectable product, thereby detecting the presence of the target nucleic acid in the sample, wherein the target nucleic acid is amplified DNA, DNA synthesized from a single-stranded RNA template, or cDNA, wherein the non-target nucleic acid part of a reporter, and wherein the polypeptide is capable of both hybridizing to the target nucleic acid and cleaving the non-target sequence. In some embodiments, the amplified DNA, DNA synthesized from single-stranded RNA template, or cDNA is double-stranded DNA, single-stranded DNA, or combinations thereof. In some embodiments, the target nucleic acid comprises any one of: a naturally occurring eukaryotic sequence, a naturally occurring prokaryotic sequence, a naturally occurring viral sequence, a naturally occurring bacterial sequence, a naturally occurring fungal sequence, an engineered eukaryotic sequence, an engineered prokaryotic sequence, an engineered viral sequence, an engineered bacterial sequence, an engineered fungal sequence, a fragment of a naturally occurring sequence, a fragment of an engineered sequence, and combinations thereof. In some embodiments, the target nucleic acid is isolated from any one of: a naturally occurring cell, a eukaryotic cell, a prokaryotic cell, a plant cell, a fungal cell, an animal cell, cell of an invertebrate, a fly cell, a cell of a vertebrate, a mammalian cell, a primate cell, a non-human primate cell, a human cell, a living cell, a non-living cell, a modified cell, a derived cell, and a non-naturally occurring cell. In some embodiments, the detectable product further comprises a detectable label or a nucleic acid comprising a detectable label selected from a reporter nucleic acid, a detection moiety, an additional polypeptide, or a combination thereof, optionally wherein the reporter nucleic acid comprises a fluorophore, a quencher, or a combination thereof.

In some embodiments, there is a threshold of detection for methods of detecting target nucleic acids. In some embodiments, methods do not detect target nucleic acids that are present in a sample or solution at a concentration less than or equal to 10 nM. For example, when a threshold of detection is 10 nM, then a signal can be detected when a target nucleic acid is present in the sample at a concentration of 10 nM or more. In some embodiments, the threshold of detection is less than or equal to 5 nM, 1 nM, 0.5 nM, 0.1 nM, 0.05 nM, 0.01 nM, 0.005 nM, 0.001 nM, 0.0005 nM, 0.0001 nM, 0.00005 nM, 0.00001 nM, 10 pM, 1 pM, 500 fM, 250 fM, 100 fM, 50 fM, 10 fM, 5 fM, 1 fM, 500 attomole (aM), 100 aM, 50 aM, 10 aM, or 1 aM. In some embodiments, the threshold of detection is in a range of from 1 aM to 1 nM, 1 aM to 500 pM, 1 aM to 200 pM, 1 aM to 100 pM, 1 aM to 10 pM, 1 aM to 1 pM, 1 aM to 500 fM, 1 aM to 100 fM, 1 aM to 1 fM, 1 aM to 500 aM, 1 aM to 100 aM, 1 aM to 50 aM, 1 aM to 10 aM, 10 aM to 1 nM, 10 aM to 500 pM, 10 aM to 200 pM, 10 aM to 100 pM, 10 aM to 10 pM, 10 aM to 1 pM, 10 aM to 500 fM, 10 aM to 100 fM, 10 aM to 1 fM, 10 aM to 500 aM, 10 aM to 100 aM, 10 aM to 50 aM, 100 aM to 1 nM, 100 aM to 500 pM, 100 pM to 200 pM, 100 aM to 100 pM, 100 aM to 10 pM, 100 aM to 1 pM, 100 aM to 500 fM, 100 aM to 100 fM, 100 aM to 1 fM, 100 aM to 500 aM, 500 aM to 1 nM, 500 aM to 500 pM, 500 aM to 200 pM, 500 aM to 100 pM, 500 aM to 10 pM, 500 aM to 1 pM, 500 aM to 500 fM, 500 aM to 100 fM, 500 aM to 1 fM, 1 fM to 1 nM, 1 fM to 500 pM, 1 fM to 200 pM, 1 fM to 100 pM, 1 fM to 10 pM, 1 fM to 1 pM, 10 fM to 1 nM, 10 fM to 500 pM, 10 fM to 200 pM, 10 fM to 100 pM, 10 fM to 10 pM, 10 fM to 1 pM, 500 fM to 1 nM, 500 fM to 500 pM, 500 fM to 200 pM, 500 fM to 100 pM, 500 fM to 10 pM, 500 fM to 1 pM, 800 fM to 1 nM, 800 fM to 500 pM, 800 fM to 200 pM, 800 fM to 100 pM, 800 fM to 10 pM, 800 fM to 1 pM, 1 pM to 1 nM, 1 pM to 500 pM, 1 pM to 200 pM, 1 pM to 100 pM, or 1 pM to 10 pM. In some embodiments, the threshold of detection of detection in a range of from 800 fM to 100 pM, 1 pM to 10 pM, 10 fM to 500 fM, 10 fM to 50 fM, 50 fM to 100 fM, 100 fM to 250 fM, or 250 fM to 500 fM. In some embodiments, the threshold of detection is in a range of from 2 aM to 100 pM, from 20 aM to 50 pM, from 50 aM to 20 pM, from 200 aM to 5 pM, or from 500 aM to 2 pM.

In some embodiments, the target nucleic acid is present in a cleavage reaction at a concentration of about 10 nM, about 20 nM, about 30 nM, about 40 nM, about 50 nM, about 60 nM, about 70 nM, about 80 nM, about 90 nM, about 100 nM, about 200 nM, about 300 nM, about 400 nM, about 500 nM, about 600 nM, about 700 nM, about 800 nM, about 900 nM, about 1 μM, about 10 μM, or about 100 μM. In some embodiments, the target nucleic acid is present in a cleavage reaction at a concentration of from 10 nM to 20 nM, from 20 nM to 30 nM, from 30 nM to 40 nM, from 40 nM to 50 nM, from 50 nM to 60 nM, from 60 nM to 70 nM, from 70 nM to 80 nM, from 80 nM to 90 nM, from 90 nM to 100 nM, from 100 nM to 200 nM, from 200 nM to 300 nM, from 300 nM to 400 nM, from 400 nM to 500 nM, from 500 nM to 600 nM, from 600 nM to 700 nM, from 700 nM to 800 nM, from 800 nM to 900 nM, from 900 nM to 1 μM, from 1 μM to 10 μM, from 10 μM to 100 μM, from 10 nM to 100 nM, from 10 nM to 1 μM, from 10 nM to 10 μM, from 10 nM to 100 μM, from 100 nM to 1 μM, from 100 nM to 10 μM, from 100 nM to 100 μM, or from 1 μM to 100 μM. In some embodiments, the target nucleic acid is present in a cleavage reaction at a concentration of from 20 nM to 50 μM, from 50 nM to 20 μM, or from 200 nM to 5 μM.

In some embodiments, methods detect a target nucleic acid in less than 60 minutes. In some embodiments, methods detect a target nucleic acid in less than about 120 minutes, less than about 110 minutes, less than about 100 minutes, less than about 90 minutes, less than about 80 minutes, less than about 70 minutes, less than about 60 minutes, less than about 55 minutes, less than about 50 minutes, less than about 45 minutes, less than about 40 minutes, less than about 35 minutes, less than about 30 minutes, less than about 25 minutes, less than about 20 minutes, less than about 15 minutes, less than about 10 minutes, less than about 5 minutes, less than about 4 minutes, less than about 3 minutes, less than about 2 minutes, or less than about 1 minute.

In some embodiments, methods require at least about 120 minutes, at least about 110 minutes, at least about 100 minutes, at least about 90 minutes, at least about 80 minutes, at least about 70 minutes, at least about 60 minutes, at least about 55 minutes, at least about 50 minutes, at least about 45 minutes, at least about 40 minutes, at least about 35 minutes, at least about 30 minutes, at least about 25 minutes, at least about 20 minutes, at least about 15 minutes, at least about 10 minutes, or at least about 5 minutes to detect a target nucleic acid. In some embodiments, the sample is contacted with the reagents for from 5 minutes to 120 minutes, from 5 minutes to 100 minutes, from 10 minutes to 90 minutes, from 15 minutes to 45 minutes, or from 20 minutes to 35 minutes.

In some embodiments, methods of detecting are performed in less than 10 hours, less than 9 hours, less than 8 hours, less than 7 hours, less than 6 hours, less than 5 hours, less than 4 hours, less than 3 hours, less than 2 hours, less than 1 hour, less than 50 minutes, less than 45 minutes, less than 40 minutes, less than 35 minutes, less than 30 minutes, less than 25 minutes, less than 20 minutes, less than 15 minutes, less than 10 minutes, less than 9 minutes, less than 8 minutes, less than 7 minutes, less than 6 minutes, or less than 5 minutes. In some embodiments, methods of detecting are performed in about 5 minutes to about 10 hours, about 10 minutes to about 8 hours, about 15 minutes to about 6 hours, about 20 minutes to about 5 hours, about 30 minutes to about 2 hours, or about 45 minutes to about 1 hour.

In some embodiments, methods comprise detecting a detectable signal within 5 minutes of contacting the sample and/or the target nucleic acid with the guide nucleic acid and/or the effector protein. In some embodiments, detecting occurs within 1, 2, 3, 4, 5, 6, 7, 8, 9, 10, 11, 12, 13, 14, 15, 20, 25, 30, 35, 40, 45, 50, 55, 60, 70, 80, 90, 100, 110, or 120 minutes of contacting the target nucleic acid. In some embodiments, detecting occurs within 1 to 120, 5 to 100, 10 to 90, 15 to 80, 20 to 60, or 30 to 45 minutes of contacting the target nucleic acid.

In some embodiments, methods of detecting as disclosed herein are compatible with methods for diagnosis of a disease or disorder.

Also described herein are methods for diagnosis comprising the use of any one of the systems described herein, any one of the kits described herein, any one of the devices described herein, or any one of the microfluidic devices described herein, wherein components of the system, kit, device, or microfluidic device further comprises a detectable label or a nucleic acid comprising a detectable label capable of hybridizing to a target nucleic acid. In some embodiments, the methods of diagnosis described herein, wherein hybridizing to a target nucleic acid results in modification of a detectable label, and wherein the detectable label emits a detectable signal upon modification. In some embodiments, the methods of diagnosis described herein, wherein the target nucleic acid is in one or more of: a naturally occurring cell, a eukaryotic cell, a prokaryotic cell, a plant cell, a fungal cell, an animal cell, cell of an invertebrate, a fly cell, a cell of a vertebrate, a mammalian cell, a primate cell, a non-human primate cell, a human cell, a living cell, a non-living cell, a modified cell, a derived cell, and a non-naturally occurring cell.

### Amplification of a Target Nucleic Acid

Methods comprise amplifying a target nucleic acid for detection using any of the compositions or systems described herein. In some embodiments, amplifying comprises changing the temperature of the amplification reaction, also known as thermal amplification (e.g., PCR). In some embodiments, amplifying is performed at essentially one temperature, also known as isothermal amplification. In some embodiments, amplifying improves at least one of sensitivity, specificity, or accuracy of the detection of the target nucleic acid.

In some embodiments, amplifying comprises subjecting a target nucleic acid to an amplification reaction selected from transcription mediated amplification (TMA), helicase dependent amplification (HDA), or circular helicase dependent amplification (cHDA), strand displacement amplification (SDA), recombinase polymerase amplification (RPA), loop mediated amplification (LAMP), exponential amplification reaction (EXPAR), rolling circle amplification (RCA), ligase chain reaction (LCR), simple method amplifying RNA targets (SMART), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), and improved multiple displacement amplification (IMDA), or any one of the amplification methods described herein.

In some embodiments, amplification of the target nucleic acid comprises modifying the nucleotide sequence of the target nucleic acid. For example, in some embodiments, amplification is used to insert a PAM sequence into a target nucleic acid that lacks a PAM sequence. In some embodiments, amplification is used to increase the homogeneity of a target nucleic acid in a sample. For example, in some embodiments, amplification is used to remove a nucleic acid variation that is not of interest in the target nucleic acid.

In some embodiments, amplifying takes 1, 2, 3, 4, 5, 6, 7, 8, 9, 10, 11, 12, 13, 14, 15, 16, 17, 18, 19, 20, 25, 30, 40, 50, or 60 minutes. In some embodiments, amplifying is performed at a temperature of around 20-45° C. In some embodiments, amplifying is performed at a temperature of less than about 20° C., less than about 25° C., less than about 30° C., less than about 35° C., less than about 37° C., less than about 40° C., or less than about 45° C. In some embodiments, the nucleic acid amplification reaction is performed at a temperature of at least about 20° C., at least about 25° C., at least about 30° C., at least about 35° C., at least about 37° C., at least about 40° C., or at least about 45° C.

### Detection of a Target Nucleic Acid

Described herein are various methods of sample amplification and detection in a single reaction volume. In some embodiments, any of the devices described herein are configured to perform amplification and detection in a same well, chamber, channel, or volume in the device. In some embodiments, methods include simultaneous amplification and detection in the same volume and/or in the same reaction. In some embodiments, methods include sequential amplification and detection in the same volume. In some embodiments, amplification and detection occur in a single reaction, where reverse transcription, amplification, in vitro transcription, or any combination thereof, and detection are carried out in a single volume. Any suitable method of reverse transcription, amplification, in vitro transcription, and detection can be used in such a reaction, such as methods of reverse transcription, amplification, in vitro transcription, and detection described herein.

In some embodiments, a DETECTR reaction is used for detecting the presence of a specific target gene in the same. In some embodiments, the DETECTR reaction produces a detectable signal, as described elsewhere herein, in the presence of a target nucleic acid sequence comprising a target gene. In some embodiments, the DETECTR reaction does not produce a signal in the absence of the target nucleic acid or in the presence of a nucleic acid sequence that does not comprise the specific mutation or comprises a different mutation. In some embodiments the mutation is a SNP. In some embodiments, a DETECTR reaction comprises a guide RNA reverse complementary to a portion of a target nucleic acid sequence comprising a specific SNP. In some embodiments, the guide RNA and the target nucleic acid comprising the specific SNP bind to and activate an effector protein, thereby producing a detectable signal as described elsewhere herein. In some embodiments, the guide RNA and a nucleic acid sequence that does not comprise the specific SNP does not bind to or activate the effector protein and does not produce a detectable signal. In some embodiments, a target nucleic acid sequence that does or does not comprise a specific SNP, is amplified using any amplification method disclosed herein. In some embodiments, the amplification reaction is combined with a reverse transcription reaction, a DETECTR reaction, or both. In some embodiments, the target nucleic acid sequence can comprise a SNP. In some embodiments, the target nucleic acid sequence can comprise a sequence indicative of a human disease.

In some embodiments, a DETECTR reaction, as described elsewhere herein, produces a detectable signal specifically in the presence of a target nucleic acid sequence comprising a target gene. In some embodiments, the target nucleic acid sequence can comprise a sequence indicative of a human disease. In some embodiments, the detectable signal produced in the DETECTR reaction is higher in the presence of a target nucleic acid comprising target nucleic acid than in the presence of a nucleic acid that does not comprise the target nucleic acid. In some embodiments, the DETECTR reaction produces a detectable signal that is at least 1-fold, at least 2-fold, at least 3-fold, at least 4-fold, at least 5-fold, at least 10-fold, at least 20-fold, at least 30-fold, at least 40-fold, at least 50-fold, at least 100-fold, at least 200-fold, at least 300-fold, at last 400-fold, at least 500-fold, at least 1000-fold, at least 2000-fold, at least 3000-fold, at least 4000-fold, at least 5000-fold, at least 6000-fold, at least 7000-fold, at least 8000-fold, at least 9000-fold, at least 10000-fold, at least 50000-fold, at least 100000-fold, at least 500000-fold, or at least 1000000-fold greater in the presence of a target nucleic acid comprising a target nucleic acid than in the presence of a nucleic acid that does not comprise the target nucleic acid. In some embodiments, the DETECTR reaction produces a detectable signal that is from 1-fold to 2-fold, from 2-fold to 3-fold, from 3-fold to 4-fold, from 4-fold to 5-fold, from 5-fold to 10-fold, from 10-fold to 20-fold, from 20-fold to 30-fold, from 30-fold to 40-fold, from 40-fold to 50-fold, from 50-fold to 100-fold, from 100-fold to 500-fold, from 500-fold to 1000-fold, from 1000-fold to 10,000-fold, from 10,000-fold to 100,000-fold, or from 100,000-fold to 1,000,000-fold greater in the presence of a target nucleic acid comprising a specific mutation or SNP than in the presence of a nucleic acid that does not comprise the specific mutation or SNP. In some embodiments, the target nucleic acid sequence can comprise a SNP. In some embodiments, the target nucleic acid sequence can comprise a sequence indicative of a human disease.

In some embodiments, a DETECTR reaction is used for detecting the presence of a target nucleic acid associated with a disease or a condition in a nucleic acid sample. In some embodiments, the DETECTR reaction reaches signal saturation within about 30 seconds, about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 75 minutes, about 80 minutes, or about 85 minutes and be used to detect the presence of a target gene associated with an increased likelihood of developing a disease or a condition in a nucleic acid sample. In some embodiments, the DETECTR reaction is used for detecting the presence of a target gene associated with a phenotype in a nucleic acid sample. For example, in some embodiments, a DETECTR reaction is used for detecting a target nucleic acid, such as a gene or exon, or a mutation of a target nucleic acid, such as a SNP, as set forth in TABLE 4 and/or TABLE 4.1. In another example, in some embodiments, a DETECTR reaction is used for detecting a target nucleic acid or a mutation of a target nucleic acid associated with any one of the diseases or disorders recited in TABLE 5 and/or TABLE 5.1. In some embodiments, a DETECTR reaction is used for detecting a SNP associated with a phenotype, for example, eye color, hair color, height, skin color, race, alcohol flush reaction, caffeine consumption, deep sleep, genetic weight, lactose intolerance, muscle composition, saturated fat and weight, or sleep movement. In some embodiments, a DETECTR reaction is used for detecting the presence of a pathological organism. In some embodiments, the pathological organism is a prokaryote, eukaryote, or a protozoa. In some embodiments, the pathological organism is a virus, an opportunistic pathogen, a parasite, a bacterium, or any combination thereof. In some embodiments, the pathological organism is SARS-CoV-2 or Streptococcus pyogenes.

### XVI. Methods of Treating a Disease or Disorder

Described herein are methods for treating a disease in a subject by contacting a target nucleic acid with a composition or system described herein, wherein the target nucleic acid is associated with a gene or expression of a gene related to the disease. In some embodiments, methods comprise treating, preventing, or inhibiting a disease or disorder associated with a mutation or aberrant expression of a gene. In some embodiments, methods for treating a disease or disorder comprise methods of editing a nucleic acid described herein. In some embodiments, compositions or systems described herein are for use in a method for treating a disease. In some embodiments, compositions or systems described herein are for use in the manufacture of a medicament for treating a disease.

In some embodiments, methods comprise administration of a composition(s) or component(s) of a system described herein. In some embodiments, the composition(s) or component(s) of the system comprises use of a recombinant nucleic acid (DNA or RNA), administered for the purpose to modify a nucleic acid. In some embodiments, the composition or component of the system comprises use of a vector to introduce a functional gene or transgene. In some embodiments, vectors comprise nonviral vectors, including cationic polymers, cationic lipids, or bio-responsive polymers. In some embodiments, the bio-responsive polymer exploits chemical-physical properties of the endosomal environment (e.g., pH) to preferentially release the genetic material in the intracellular space. In some embodiments, vectors comprise viral vectors, including retroviruses, adenoviruses, adeno-associated viruses, and herpes simplex viruses. In some embodiments, the vector comprises a replication-defective viral vector, comprising an insertion of a therapeutic gene inserted in genes essential to the lytic cycle, preventing the virus from replicating and exerting cytotoxic effects. By way of non-limiting example, in some embodiments, the composition(s) comprises pharmaceutical compositions described herein. Methods of gene therapy that are applicable to the compositions and systems described herein are described in more detail in Ingusci et al., “Gene Therapy Tools for Brain Diseases”, Front. Pharmacol. 10:724 (2019), which is hereby incorporated by reference in its entirety.

In some embodiments, treating, preventing, or inhibiting disease or disorder in a subject comprises contacting a target nucleic acid associated with a particular ailment with a composition described herein. In some embodiments, the methods of treating, preventing, or inhibiting a disease or disorder involves removing, editing, modifying, replacing, transposing, or affecting the regulation of a genomic sequence of a patient in need thereof. In some embodiments, the methods of treating, preventing, or inhibiting a disease or disorder involves modulating gene expression.

In some embodiments, the compositions and systems described herein are for use in therapy. In some embodiments, the compositions and systems described herein are for use in treating a disease or condition described herein. Also provided is the use of the compositions described herein in the manufacture of a medicament. Also provided is the use of the compositions described herein in the manufacture of a medicament for therapeutic and/or prophylactic treatment of a disease or condition described herein.

In some embodiments, the polypeptides (e.g., effector proteins, effector partners, fusion partners, fusion proteins, or combination thereof) described herein are for use in therapy. In some embodiments, the polypeptides described herein are for use in treating a disease or condition described herein. Also provided is the use of the polypeptides described herein in the manufacture of a medicament. Also provided is the use of the polypeptides described herein in the manufacture of a medicament for therapeutic and/or prophylactic treatment of a disease or condition described herein.

In some embodiments, the guide nucleic acids described herein are for use in therapy. In some embodiments, the guide nucleic acids described herein are for use in treating a disease or condition described herein. Also provided is the use of the guide nucleic acids described herein in the manufacture of a medicament. Also provided is the use of the guide nucleic acids described herein in the manufacture of a medicament for therapeutic and/or prophylactic treatment of a disease or condition described herein.

Described herein are compositions, systems, and methods for treating a disease in a subject by modifying a target nucleic acid associated with a gene or expression of a gene related to the disease. For example, in some embodiments, the editing comprises knock-down of expression of a gene that produces the target nucleic acid. In some embodiments, the compositions, systems and methods comprise LNPs, wherein the LNPs comprise the effector proteins described herein or nucleic acids encoding the effector proteins, the fusion partners described herein or nucleic acids encoding the fusion partners, the fusion proteins described herein or nucleic acids encoding the fusion proteins, or combinations thereof. In some embodiments, the LNPs comprise chemically modified guide nucleic acids. In some embodiments, the LNPs described herein are used for delivering the compositions, or one or more components of the systems described herein to a specific organ (e.g., liver). Alternatively, in some embodiments, the compositions, systems and methods comprise AAV particles, wherein the AAV particles comprise nucleic acids encoding the effector proteins described herein, the fusion partners described herein, the fusion proteins described herein, or combinations thereof. In some embodiments, the AAV particles comprise nucleic acids encoding guide nucleic acids described herein. In some embodiments, the AAV particles described herein are used for delivering the compositions, or one or more components of the systems described herein to specific cells (e.g., nerve cells or muscle cells). In some embodiments, methods comprise administering a composition or cell described herein to a subject. By way of non-limiting example, in some embodiments, the disease comprises a cancer, an ophthalmological disorder, a neurological disorder, a neurodegenerative disease, a blood disorder, or a metabolic disorder, or a combination thereof. In some embodiments, the disease comprises an inherited disorder, also referred to as a genetic disorder. In some embodiments, the disease is the result of an infection or associated with an infection. Also, by way of non-limiting example, the compositions are pharmaceutical compositions described herein.

Also described herein are methods of treating a disease or disorder associated with a mutation or aberrant expression of a gene in a subject in need thereof, the methods comprising administering to the subject the pharmaceutical composition described herein.

In some embodiments, the compositions and methods described herein are used for treating, preventing, or inhibiting a disease or syndrome in a subject. In some embodiments, the disease is a liver disease, a lung disease, an eye disease, or a muscle disease. Exemplary diseases and syndromes include but are not limited to the diseases and syndromes listed in TABLE 5 and TABLE 5.1.

In some embodiments, compositions and methods modify at least one nucleic acid associated to a gene associated with a disease described herein or the expression thereof. In some embodiments, the disease comprises Alzheimer's disease and the gene is selected from APP, BACE-1, PSD95, MAPT, PSEN1, PSEN2, TARDBP, APOE, and APOEε4. In some embodiments, the disease comprises Parkinson's disease and the gene is selected from SNCA, GDNF, and LRRK2. In some embodiments, the disease comprises dementia and the gene is TARDBP. In some embodiments, the disease comprises Pick's Disease and the gene is TARDBP. In some embodiments, the disease comprises congenital muscular dystrophy 1A (MDC1A) and the gene is LAMA1 or LAMA2. In some embodiments, the disease comprises Ullrich Congenital Muscular Dystrophy (UCMD) and the gene is selected from COL6A1, COL6A2 and COL6A3. In some embodiments, the disease comprises Limb Girdle Muscular Dystrophies (LGMD1B, LGMD2A, LGMD2B) and the gene is selected from LMNA, DYSF, and CAPN3. In some embodiments, the disease comprises Nemaline Myopathy and the gene is selected from ACTA1, NEB, TPM2, TPM3, TNNT1, TNNT3, TNNI2 and LMOD3. In some embodiments, the disease comprises Centronuclear myopathy and the gene is DNM2. In some embodiments, the disease comprises Huntington's disease and the gene is H7T. In some embodiments, the disease comprises Alpha-1 antitrypsin deficiency (AATD) and the gene is SERPINA. In some embodiments, the disease comprises amyotrophic lateral sclerosis (ALS) and the gene is selected from SOD), FUS, C90RF72, ATXN2, TARDBP, and CHCHD10. In some embodiments, the disease comprises Alexander Disease and the gene is GFAP. In some embodiments, the disease comprises anaplastic large cell lymphoma and the gene is CD30. In some embodiments, the disease comprises Angelman Syndrome and the gene is UBE3A. In some embodiments, the disease comprises calcific aortic stenosis and the gene is Apo(a). In some embodiments, the disease comprises CD3Z-associated primary T-cell immunodeficiency and the gene is CD3Z or CD247. In some embodiments, the disease comprises CD18 deficiency and the gene is ITGB2. In some embodiments, the disease comprises CD40L deficiency and the gene is CD40L. In some embodiments, the disease comprises congenital adrenal hyperplasia and the gene is CAH1. In some embodiments, the disease comprises CNS trauma and the gene is VEGF. In some embodiments, the disease comprises coronary heart disease and the gene is selected from FGA, FGB, and FGG. In some embodiments, the disease comprises MECP2 Duplication syndrome and Rett syndrome and the gene is MECP2. In some embodiments, the disease comprises a bleeding disorder (coagulation) and the gene is FXI. In some embodiments, the disease comprises fragile X syndrome and the gene is FMR1. In some embodiments, the disease comprises Fuchs corneal dystrophy and the gene is selected from ZEB1, SLC4A11, and LOXHD1. In some embodiments, the disease comprises GM2-Gangliosidoses (e.g., Tay Sachs Disease, Sandhoff disease) and the gene is selected from HEXA and HEXB. In some embodiments, the disease comprises Hearing loss disorders and the gene is DFNA36. In some embodiments, the disease comprises Pompe disease, including infantile onset Pompe disease (IOPD) and late onset Pompe disease (LOPD) and the gene is GAA. In some embodiments, the disease comprises Retinitis pigmentosa and the gene is selected from PDE6B, RHO, RP1, RP2, RPGR, PRPH2, IMPDH1, PRPF31, CRB1, PRPF8, TULP1, CA4, HPRPF3, ABCA4, EYS, CERKL, FSCN2, TOPORS, SNRNP200, PRCD, NR2E3, MERTK, USH2A, PROM1, KLHL7, CNGB1, TTC8, ARL6, DHDDS, BEST1, LRAT, SPARA7, CRX, CLRN1, RPE65, and WDR19. In some embodiments, the disease comprises Leber Congenital Amaurosis Type 10 and the gene is CEP290. In some embodiments, the disease comprises cardiovascular disease and/or lipodystrophies and the gene is selected from ABCG5, ABCG8, AGT, ANGPTL3, APOCIII, APOA1, APOE, APOL1, ARH, CDKN2B, CFB, CXCL12, FXI, FXII, GATA-4, MIA3, MKL2, MTHFD1L, MYH7, NKX2-5, NOTCH1, PKK, PCSK9, PSRC1, SMAD3, and TTR. In some embodiments, the disease comprises cardiovascular disease and/or lipodystrophies and the gene is ANGPTL3. In some embodiments, the disease comprises cardiovascular disease and/or lipodystrophies and the gene is PCSK9. In some embodiments, the disease comprises cardiovascular disease and/or lipodystrophies and the gene is TTR. In some embodiments, the disease comprises severe hypertriglyceridemia (SHTG) and the gene is APOCIII or ANGPTL4. In some embodiments, the disease comprises acromegaly and the gene is GHR. In some embodiments, the disease comprises acute myeloid leukemia and the gene is CD22. In some embodiments, the disease comprises diabetes and the gene is selected from ANGPTL7 and GCGR. In some embodiments the disease comprises insulin resistance and the gene is ANGPTL7. In some embodiments, the disease comprises glaucoma and the gene is selected from ANGPTL7 and MYOC. In some embodiments, the disease comprises NAFLD/NASH and the gene is selected from HSD17B13, PSD3, GPAM, CIDEB, DGAT2 and PNPLA3. In some embodiments, the disease comprises NASH/cirrhosis and the gene is MARC1. In some embodiments, the disease comprises cancer and the gene is selected from STAT3, YAP1, FOXP3, AR (Prostate cancer), and IRF4 (multiple myeloma). In some embodiments, the disease comprises cystic fibrosis and the gene is CFTR. In some embodiments, the disease comprises Duchenne muscular dystrophy and the gene is DMD. In some embodiments, the disease comprises ornithine transcarbamylase deficiency (OTCD) and the gene is OTC. In some embodiments, the disease comprises congenital adrenal hyperplasia (CAH) and the gene is CYP21A2. In some embodiments, the disease comprises atherosclerotic cardiovascular disease (ASCVD) and the gene is LPA. In some embodiments, the disease comprises hepatitis B virus infection (CHB) and the gene is HBV covalently closed circular DNA (cccDNA). In some embodiments, the disease comprises citrullinemia type I and the gene is ASS). In some embodiments, the disease comprises citrullinemia type I and the gene is SLC25A13. In some embodiments, the disease comprises citrullinemia type I and the gene is ASS). In some embodiments, the disease comprises arginase-1 deficiency and the gene is ARG1. In some embodiments, the disease comprises carbamoyl phosphate synthetase I deficiency and the gene is CPS1. In some embodiments, the disease comprises argininosuccinic aciduria and the gene is ASL. In some embodiments, the disease comprises angioedema and the gene is PKK. In some embodiments, the disease comprises thalassemia and the gene is TMPRSS6. In some embodiments, the disease comprises achondroplasia and the gene is FGFR3. In some embodiments, the disease comprises Cri du chat syndrome and the gene is selected from CTNND2. In some embodiments, the disease comprises sickle cell anemia and the gene is Beta globin gene. In some embodiments, the disease comprises Alagille Syndrome and the gene is selected from JAG1 and NOTCH2. In some embodiments, the disease comprises Charcot-Marie-Tooth disease and the gene is selected from PMP22 and MFN2. In some embodiments, the disease comprises Crouzon syndrome and the gene is selected from FGFR2, FGFR3, and FGFR3. In some embodiments, the disease comprises Dravet Syndrome and the gene is selected from SCN1A and SCN2A. In some embodiments, the disease comprises Emery-Dreifuss syndrome and the gene is selected from EMD, LMNA, SYNE1, SYNE2, FHL1, and TMEM43. In some embodiments, the disease comprises Factor V Leiden thrombophilia and the gene is F5. In some embodiments, the disease comprises fabry disease and the gene is GLA. In some embodiments, the disease comprises facioscapulohumeral muscular dystrophy and the gene is selected from FSHD1 and DUX4. In some embodiments, the disease comprises Fanconi anemia and the gene is selected from FANCA, FANCB, FANCC, FANCD1, FANCD2, FANCE, FANCF, FANCG, FANCI, FANCJ, FANCL, FANCM, FANCN, FANCP, FANCS, RAD51C, and XPF. In some embodiments, the disease comprises Familial Creutzfeld-Jakob disease and the gene is PRNP. In some embodiments, the disease comprises Familial Mediterranean Fever and the gene is MEFV. In some embodiments, the disease comprises Friedreich's ataxia and the gene is FXN. In some embodiments, the disease comprises Gaucher disease and the gene is GBA. In some embodiments, the disease comprises human papilloma virus (HPV) infection and the gene is HPV E7. In some embodiments, the disease comprises hemochromatosis and the gene is HFE, optionally comprising a C282Y mutation. In some embodiments, the disease comprises Hemophilia A and the gene is FVHIII. In some embodiments, the disease comprises hereditary angioedema and the gene is SERPING1 or KLKB1. In some embodiments, the disease comprises histiocytosis and the gene is CDL. In some embodiments, the disease comprises immunodeficiency 17 and the gene is CD3D. In some embodiments, the disease comprises immunodeficiency 13 and the gene is CD4. In some embodiments, the disease comprises Common Variable Immunodeficiency and the gene is selected from CD19 and CD81. In some embodiments, the disease comprises Joubert syndrome and the gene is selected from INPP5E, TMEM216, AHI1, NPHP1, CEP290, TMEM67, RPGRIP1L. ARL13B, CC2D2A, OFD1, TMEM138, TCTN3, ZNF423, and AMRC9. In some embodiments, the disease comprises leukocyte adhesion deficiency and the gene is CD18. In some embodiments, the disease comprises Li-Fraumeni syndrome and the gene is TPS3. In some embodiments, the disease comprises lymphoproliferative syndrome and the gene is CD27. In some embodiments, the disease comprises Lynch syndrome and the gene is selected from MSH2, MLH1, MSH6, PMS2, PMS1, TGFBR2, and MLH3. In some embodiments, the disease comprises mantle cell lymphoma and the gene is CD5. In some embodiments, the disease comprises Marfan syndrome and the gene is FBN1. In some embodiments, the disease comprises mastocytosis and the gene is CD2. In some embodiments, the disease comprises methylmalonic acidemia and the gene is selected from MMAA, MMAB, and MUT. In some embodiments, the disease comprises mycosis fungoides and the gene is CD7. In some embodiments, the disease comprises myotonic dystrophy and the gene is selected from CNBP and DMPK. In some embodiments, the disease comprises neurofibromatosis and the gene is selected from NF1, and NF2. In some embodiments, the disease comprises osteogenesis imperfecta and the gene is selected from COL1A1, COL1A2, and IFITM5. In some embodiments, the disease comprises non-small cell lung cancer and the gene is selected from KRAS, EGFR, ALK, METex14, BRAF V600E, ROSI, RET, and NTRK. In some embodiments, the disease comprises subependymal glioma and the gene is RPTOR. In some embodiments, the disease comprises Peutz-Jeghers syndrome and the gene is STK11. In some embodiments, the disease comprises polycystic kidney disease and the gene is selected from PKD1 and PKD2. In some embodiments, the disease comprises Pitt-Hopkins Syndrome and the gene is TCF4. In some embodiments, the disease comprises Severe Combined Immune Deficiency and the gene is selected from IL7R, RAG1, and JAK3. In some embodiments, the disease comprises PRKAG2 cardiac syndrome and the gene is PRKAG2. In some embodiments, the disease comprises spinocerebellar ataxia and the gene is selected from ATXN1, ATXN2, ATXN3, PLEKHG4, SPTBN2, CACNA1A, ATXN7, ATXN8OS, ATXN10, TTBK2, PPP2R2B, KCNC3, PRKCG, ITPR1, TBP, KCND3, and FGF14. In some embodiments, the disease comprises thrombophilia due to antithrombin III deficiency and the gene is SERPINC1. In some embodiments the disease comprises spinal muscular atrophy and the gene is SMN1. In some embodiments, the disease comprises Usher Syndrome and the gene is selected from MYO7A, USH1C, CDH23, PCDH15, USH1G, USH2A, GPR98, DFNB31, and CLRN1. In some embodiments, the disease comprises von Willebrand disease and the gene is VWF. In some embodiments, the disease comprises Waardenburg syndrome and the gene is selected from PAX3, MITF, WS2B, WS2C, SNAI2, EDNRB, EDN3, and SOX10. In some embodiments, the disease comprises Wiskott-Aldrich Syndrome and the gene is WAS. In some embodiments, the disease comprises von Hippel-Lindau disease and the gene is VHL. In some embodiments, the disease comprises Wilson disease and the gene is ATP7B. In some embodiments, the disease comprises Zellweger syndrome and the gene is selected from PEX1, PEX2, PEX3, PEX5, PEX6, PEX10, PEX12, PEX13, PEX14, PEX16, PEX19, and PEX26. In some embodiments, the disease comprises infantile myofibromatosis and the gene is CD34. In some embodiments, the disease comprises platelet glycoprotein IV deficiency and the gene is CD36. In some embodiments, the disease comprises immunodeficiency with hyper-IgM type 3 and the gene is CD40. In some embodiments, the disease comprises hemolytic uremic syndrome and the gene is CD46. In some embodiments, the disease comprises complement hyperactivation, angiopathic thrombosis, or protein-losing enteropathy and the gene is CD55. In some embodiments, the disease comprises hemolytic anemia and the gene is CD59. In some embodiments, the disease comprises calcification of joints and arteries and the gene is CD73. In some embodiments, the disease comprises immunoglobulin alpha deficiency and the gene is CD79A. In some embodiments, the disease comprises C syndrome and the gene is CD96. In some embodiments, the disease comprises pain and the gene is NAV1.7. In some embodiments, the disease comprises hairy cell leukemia and the gene is CD123. In some embodiments, the disease comprises histiocytic sarcoma and the gene is CD163. In some embodiments, the disease comprises autosomal dominant deafness and the gene is CD164. In some embodiments, the disease comprises immunodeficiency 25 and the gene is CD247. In some embodiments, the disease comprises methymalonic acidemia due to transcobalamin receptor defect and the gene is CD320.

### Cancer

In some embodiments, the disease comprises cancer. Non-limiting examples of cancers include: acute lymphoblastic leukemia; acute lymphoblastic lymphoma; acute lymphocytic leukemia; acute myelogenous leukemia; acute myeloid leukemia (adult/childhood); adrenocortical carcinoma; AIDS-related cancers; AIDS-related lymphoma; anal cancer; appendix cancer; astrocytoma; atypical teratoid/rhabdoid tumor; basal-cell carcinoma; bile duct cancer; extrahepatic (cholangiocarcinoma); bladder cancer; bone osteosarcoma/malignant fibrous histiocytoma; brain cancer (adult/childhood); brain tumor; cerebellar astrocytoma (adult/childhood); brain tumor, cerebral astrocytoma/malignant glioma brain tumor; brain tumor, ependymoma; brain tumor, medulloblastoma; brain tumor, supratentorial primitive neuroectodermal tumors; brain tumor, visual pathway and hypothalamic glioma; brainstem glioma; breast cancer; bronchial adenomas/carcinoids; bronchial tumor; Burkitt lymphoma; cancer of childhood; carcinoid gastrointestinal tumor; carcinoid tumor; carcinoma of adult, unknown primary site; carcinoma of unknown primary; central nervous system embryonal tumor; central nervous system lymphoma, primary; cervical cancer; childhood adrenocortical carcinoma; childhood cancers; childhood cerebral astrocytoma; chordoma, childhood; chronic lymphocytic leukemia; chronic myelogenous leukemia; chronic myeloid leukemia; chronic myeloproliferative disorders; colon cancer; colorectal cancer; craniopharyngioma; cutaneous T-cell lymphoma; desmoplastic small round cell tumor; emphysema; endometrial cancer; ependymoblastoma; ependymoma; esophageal cancer; Ewing sarcoma in the Ewing family of tumors; extracranial germ cell tumor; extragonadal germ cell tumor; extrahepatic bile duct cancer; gallbladder cancer; gastric (stomach) cancer; gastric carcinoid; gastrointestinal carcinoid tumor; gastrointestinal stromal tumor; germ cell tumor: extracranial, extragonadal, or ovarian gestational trophoblastic tumor; gestational trophoblastic tumor, unknown primary site; glioma; glioma of the brain stem; glioma, childhood visual pathway and hypothalamic; hairy cell leukemia; head and neck cancer; heart cancer; hepatocellular (liver) cancer; Hodgkin's lymphoma; hypopharyngeal cancer; hypothalamic and visual pathway glioma; intraocular melanoma; islet cell carcinoma (endocrine pancreas); Kaposi Sarcoma; kidney cancer (renal cell cancer); Langerhans cell histiocytosis; laryngeal cancer; lip and oral cavity cancer; liposarcoma; liver cancer (primary); lung cancer, non-small cell; lung cancer, small cell; lymphoma, primary central nervous system; macroglobulinemia, Waldenstrom; male breast cancer; malignant fibrous histiocytoma of bone/osteosarcoma; medulloblastoma; medulloepithelioma; melanoma; melanoma, intraocular (eye); Merkel cell cancer; Merkel cell skin carcinoma; mesothelioma; mesothelioma, adult malignant; metastatic squamous neck cancer with occult primary; mouth cancer; multiple endocrine neoplasia syndrome; multiple myeloma/plasma cell neoplasm; mycosis fungoides, myelodysplastic syndromes; myelodysplastic/myeloproliferative diseases; myelogenous leukemia, chronic; myeloid leukemia, adult acute; myeloid leukemia, childhood acute; myeloma, multiple (cancer of the bone-marrow); myeloproliferative disorders, chronic; nasal cavity and paranasal sinus cancer; nasopharyngeal carcinoma; neuroblastoma, non-small cell lung cancer; non-Hodgkin's lymphoma; oligodendroglioma; oral cancer; oral cavity cancer; oropharyngeal cancer; osteosarcoma/malignant fibrous histiocytoma of bone; ovarian cancer; ovarian epithelial cancer (surface epithelial-stromal tumor); ovarian germ cell tumor; ovarian low malignant potential tumor; pancreatic cancer; pancreatic cancer, islet cell; papillomatosis; paranasal sinus and nasal cavity cancer; parathyroid cancer; penile cancer; pharyngeal cancer; pheochromocytoma; pineal astrocytoma; pineal germinoma; pineal parenchymal tumors of intermediate differentiation; pineoblastoma and supratentorial primitive neuroectodermal tumors; pituitary tumor; pituitary adenoma; plasma cell neoplasia/multiple myeloma; pleuropulmonary blastoma; primary central nervous system lymphoma; prostate cancer; rectal cancer; renal cell carcinoma (kidney cancer); renal pelvis and ureter, transitional cell cancer; NUT midline carcinoma; retinoblastoma; rhabdomyosarcoma, childhood; salivary gland cancer; sarcoma, Ewing family of tumors; Sézary syndrome; skin cancer (melanoma); skin cancer (non-melanoma); small cell lung cancer; small intestine cancer soft tissue sarcoma; soft tissue sarcoma; spinal cord tumor; squamous cell carcinoma; squamous neck cancer with occult primary, metastatic; stomach (gastric) cancer; subependymal glioma; supratentorial primitive neuroectodermal tumor; T-cell lymphoma, cutaneous (Mycosis Fungoides and Sézary syndrome); testicular cancer; throat cancer; thymoma; thymoma and thymic carcinoma; thyroid cancer; thyroid cancer, childhood; transitional cell cancer of the renal pelvis and ureter; urethral cancer; uterine cancer, endometrial; uterine sarcoma; vaginal cancer; vulvar cancer; and Wilms Tumor.

In some embodiments, mutations are associated with cancer or are causative of cancer. The target nucleic acid, in some embodiments, comprises a portion of a gene comprising a mutation associated with cancer, a gene whose overexpression is associated with cancer, a tumor suppressor gene, an oncogene, a checkpoint inhibitor gene, a gene associated with cellular growth, a gene associated with cellular metabolism, a gene associated with cell cycle, or combinations thereof. Non-limiting examples of genes comprising a mutation associated with cancer are ABL, ACE, AF4/HRX, AKT-2, ALK, ALK/NPM, AML), AML1/MTG8, APC, ATM, AXIN2, AXL, BAP1, BARD1, BCL-2, BCL-3, BCL-6, BCR/ABL, BLM, BMPR1A, BRCA1, BRCA2, BRIP1, c-MYC, CASR, CCRS, CDC73, CDH1, CDK4, CDKN1B, CDKN1C, CDKN2A, CEBPA, CHEK2, CREBBP, CTNNA1, DBL, DEK/CAN, DICER1, DIS3L2, E2A/PBX1, EGFR, ENL/HRX, EPCAM, ERG/TLS, ERBB, ERBB-2, ETS-1, EWS/FLI-1, FH, FKRP, FLCN, FMS, FOS, FPS, GATA2, GCG, GLI, GPC3, GPGSP, GREM1, HER2/neu, HOX11, HOXB13, HRAS, HST, IL-3, INT-2, JAK1, JUN, KIT, KS3, K-SAM, LBC, LCK, LMO1, LMO2, L-MYC, LYL-1, LYT-10, LYT-10/Cα1, MAS, MAX, MDM-2, MEN1, MET, MITF, MLH1, MLL, MOS, MSH1, MSH2, MSH3, MSH6, MTG8/AML1, MUTYH, MYB, MYH11/CBFB, NBN, NEU, NF1, NF2, N-MYC, NTHL1, OST, PALB2, PAX-5, PBX1/E2A, PCDC1, PDGFRA, PHOX2B, PIM-1, PMS2, POLD1, POLE, POT1, PPARG, PRAD-1, PRKAR1A, PTCH1, PTEN, RAD50, RAD51C, RAD5D, RAF, RAR/PML, RAS-H, RAS-K, RAS-N, RB1, RECQL4, REL/NRG, RET, RHOM1, RHOM2, ROS, RPTOR, RUNX1, SDHA, SDHAF, SDHAF2, SDHB, SDHC, SDHD, SET/CAN, SIS, SKI, SMAD4, SMARCA4, SMARCB1, SMARCE1, SRC, STK11, SUFU, TAL1, TAL2, TAN-1, TIAM1, TERC, TERT, TIMP3, TMEM127, TNF, TP53, TRAC, TSC1, TSC2, TRK, VHL, WRN, and WT1. Non-limiting examples of oncogenes are KRAS, NRAS, BRAF, MYC, CTNNB1, and EGFR. In some embodiments, the oncogene is a gene that encodes a cyclin dependent kinase (CDK). Non-limiting examples of CDKs are Cdk1, Cdk4, Cdk5, Cdk7, Cdk8, Cdk9, Cdk11 and Cdk20. Non-limiting examples of tumor suppressor genes are TPS3, RB1, and PTEN.

### Infections

Described herein are compositions, systems, and methods for treating an infection in a subject. In some embodiments, infections are caused by a pathogen (e.g., bacteria, viruses, fungi, and parasites). In some embodiments, compositions, systems, and methods modify a target nucleic acid associated with the pathogen or parasite causing the infection. In some embodiments, the target nucleic acid is in the pathogen or parasite itself or in a cell, tissue or organ of the subject that the pathogen or parasite infects. In some embodiments, the methods described herein include treating an infection caused by one or more bacterial pathogens. Non-limiting examples of bacterial pathogens include Acholeplasma laidlawii, Brucella abortus, Chlamydia psittaci, Chlamydia trachomatis, Cryptococcus neoformans, Escherichia coli, Legionella pneumophila, Lyme disease spirochetes, methicillin-resistant Staphylococcus aureus, Mycobacterium leprae, Mycobacterium tuberculosis, Mycoplasma arginini, Mycoplasma arthritidis, Mycoplasma genitalium, Mycoplasma hyorhinis, Mycoplasma orale, Mycoplasma pneumoniae, Mycoplasma salivarium, Neisseria gonorrhoeae, Neisseria meningitidis, Pneumococcus, Pseudomonas aeruginosa, sexually transmitted infection, Streptococcus agalactiae, Streptococcus pyogenes, and Treponema pallidum.

In some embodiments, compositions, systems or methods described herein treat an infection caused by one or more viral pathogens. Non-limiting examples of viral pathogens include adenovirus, blue tongue virus, chikungunya, coronavirus (e.g., SARS-CoV-2), cytomegalovirus, Dengue virus, Ebola, Epstein-Barr virus, feline leukemia virus, Hemophilus influenzae B, Hepatitis Virus A, Hepatitis Virus B, Hepatitis Virus C, herpes simplex virus I, herpes simplex virus II, human papillomavirus (HPV) including HPV16 and HPV18, human serum parvo-like virus, human T-cell leukemia viruses, immunodeficiency virus (e.g., HIV), influenza virus, lymphocytic choriomeningitis virus, measles virus, mouse mammary tumor virus, mumps virus, murine leukemia virus, polio virus, rabies virus, Reovirus, respiratory syncytial virus (RSV), rubella virus, Sendai virus, simian virus 40, Sindbis virus, varicella-zoster virus, vesicular stomatitis virus, wart virus, West Nile virus, yellow fever virus, or any combination thereof.

In some embodiments, compositions, systems or methods described herein treat an infection caused by one or more parasites. Non-limiting examples of parasites include helminths, annelids, platyhelminthes, nematodes, and thorny-headed worms. In some embodiments, parasitic pathogens comprise, without limitation, Babesia bovis, Echinococcus granulosus, Eimeria tenella, Leishmania tropica, Mesocestoides cord, Onchocerca volvulus, Plasmodium falciparum, Plasmodium vivax, Schistosoma japonicum, Schistosoma mansoni, Schistosoma spp., Taenia hydatigena, Taenia ovis, Taenia saginata, Theileria parva, Toxoplasma gondii, Toxoplasma spp., Trichinella spiralis, Trichomonas vaginalis, Trypanosoma brucei, Trypanosoma cruzi, Trypanosoma rangeli, Trypanosoma rhodesiense, Balantidium coli, Entamoeba histolytica, Giardia spp., Isospora spp., Trichomonas spp., or any combination thereof.

### Genetically Modified Cells and Organisms

In some embodiments, methods of modifying described herein is employed to generate a genetically modified cell. In some embodiments, the cell is a eukaryotic cell (e.g., a mammalian cell) or a prokaryotic cell (e.g., an archaeal cell). In some embodiments, the cell is derived from a multicellular organism and cultured as a unicellular entity. In some embodiments, the cell comprises a heritable genetic modification, such that progeny cells derived therefrom comprise the heritable genetic mutation. In some embodiments, the cell is progeny of a genetically modified cell comprising a genetic modification of the genetically modified parent cell. In some embodiments, the genetically modified cell comprises a deletion, insertion, mutation, or non-native sequence relative to a wild-type version of the cell or the organism from which the cell was derived.

In some embodiments, methods of modifying described herein is performed in a cell. In some embodiments, the cell is in vitro. In some embodiments, the cell is in vivo. In some embodiments, the cell is ex vivo. In some embodiments, the cell is an isolated cell. In some embodiments, the cell is inside of an organism. In some embodiments, the cell is an organism. In some embodiments, the cell is in a cell culture. In some embodiments, the cell is one of a collection of cells. In some embodiments, the cell is a mammalian cell or derived there from. In some embodiments, the cell is a rodent cell or derived there from. In some embodiments, the cell is a human cell or derived there from. In some embodiments, the cell is a eukaryotic cell or derived there from. In some embodiments, the cell is a progenitor cell or derived there from. In some embodiments, the cell is a pluripotent stem cell or derived there from. In some embodiments, the cell is an animal cell or derived there from. In some embodiments, the cell is an invertebrate cell or derived there from. In some embodiments, the cell is a vertebrate cell or derived there from. In some embodiments, the cell is from a specific organ or tissue. In some embodiments, the cell is a hepatocyte. In some embodiments, the tissue is a subject's blood, bone marrow, or cord blood. In some embodiments, the tissue is a heterologous donor blood, cord blood, or bone marrow. In some embodiments, the tissue is an allogenic blood, cord blood, or bone marrow. In some embodiments, the tissue is muscle. In some embodiments, the muscle is a skeletal muscle. In some embodiments, skeletal muscles include the following: abductor digiti minimi (foot), abductor digiti minimi (hand), abductor hallucis, abductor pollicis brevis, abductor pollicis longus, adductor brevis, adductor hallucis, adductor longus, adductor magnus, adductor pollicis, anconeus, articularis cubiti, articularis genu, aryepiglotticus, auricularis, biceps brachii, biceps femoris, brachialis, brachioradialis, buccinator, bulbospongiosus, constrictor of pharynx—inferior, constrictor of pharynx—middle, constrictor of pharynx—superior, coracobrachialis, corrugator supercilii, cremaster, cricothyroid, dartos, deep transverse perinei, deltoid, depressor anguli oris, depressor labii inferioris, diaphragm, digastric, digastric (anterior view), erector spinae—spinalis, erector spinae—iliocostalis, erector spinae—longissimus, extensor carpi radialis brevis, extensor carpi radialis longus, extensor carpi ulnaris, extensor digiti minimi (hand), extensor digitorum (hand), extensor digitorum brevis (foot), extensor digitorum longus (foot), extensor hallucis brevis, extensor hallucis longus, extensor indicis, extensor pollicis brevis, extensor pollicis longus, external oblique abdominis, flexor carpi radialis, flexor carpi ulnaris, flexor digiti minimi brevis (foot), flexor digiti minimi brevis (hand), flexor digitorum brevis, flexor digitorum longus (foot), flexor digitorum profundus, flexor digitorum superficialis, flexor hallucis brevis, flexor hallucis longus, flexor pollicis brevis, flexor pollicis longus, frontalis, gastrocnemius, gemellus inferior, gemellus superior, genioglossus, geniohyoid, gluteus maximus, gluteus medius, gluteus minimus, gracilis, hyoglossus, iliacus, inferior oblique, inferior rectus, infraspinatus, intercostals external, intercostals innermost, intercostals internal, internal oblique abdominis, interossei—dorsal of hand, interossei—dorsal of foot, interossei—palmar of hand, interossei—plantar of foot, interspinales, intertransversarii, intrinsic muscles of tongue, ishiocavernosus, lateral cricoarytenoid, lateral pterygoid, lateral rectus, latissimus dorsi, levator anguli oris, levator ani-coccygeus, levator ani—iliococcygeus, levator ani-pubococcygeus, levator ani-puborectalis, levator ani-pubovaginalis, levator labii superioris, levator labii superioris, alaeque nasi, levator palpebrae superioris, levator scapulae, levator veli palatini, levatores costarum, longus capitis, longus colli, lumbricals of foot, lumbricals of hand, masseter, medial pterygoid, medial rectus, mentalis, m. uvulae, mylohyoid, nasalis, oblique arytenoid, obliquus capitis inferior, obliquus capitis superior, obturator externus, obturator internus (A), obturator internus (B), omohyoid, opponens digiti minimi (hand), opponens pollicis, orbicularis oculi, orbicularis oris, palatoglossus, palatopharyngeus, palmaris brevis, palmaris longus, pectineus, pectoralis major, pectoralis minor, peroneus brevis, peroneus longus, peroneus tertius, piriformis (A), piriformis (B), plantaris, platysma, popliteus, posterior cricoarytenoid, procerus, pronator quadratus, pronator teres, psoas major, psoas minor, pyramidalis, quadratus femoris, quadratus lumborum, quadratus plantae, rectus abdominis, rectus capitus anterior, rectus capitus lateralis, rectus capitus posterior major, rectus capitus posterior minor, rectus femoris, rhomboid major, rhomboid minor, risorius, salpingopharyngeus, sartorius, scalenus anterior, scalenus medius, scalenus minimus, scalenus posterior, semimembranosus, semitendinosus, serratus anterior, serratus posterior inferior, serratus posterior superior, soleus, sphincter ani, sphincter urethrae, splenius capitis, splenius cervicis, stapedius, sternocleidomastoid, sternohyoid, sternothyroid, styloglossus, stylohyoid, stylohyoid (anterior view), stylopharyngeus, subclavius, subcostalis, subscapularis, superficial transverse perinei, superior oblique, superior rectus, supinator, supraspinatus, temporalis, temporoparietalis, tensor fasciae lata, tensor tympani, tensor veli palatini, teres major, teres minor, thyro-arytenoid & vocalis, thyro-epiglotticus, thyrohyoid, tibialis anterior, tibialis posterior, transverse arytenoid, transversospinalis—multifidus, transversospinalis—rotatores, transversospinalis—semispinalis, transversus abdominis, transversus thoracis, trapezius, triceps, vastus intermedius, vastus lateralis, vastus medialis, zygomaticus major, or zygomaticus minor. In some embodiments, the cell is a myocyte. In some embodiments, the cell is a muscle cell. In some embodiments, the muscle cell is a skeletal muscle cell. In some embodiments, the skeletal muscle cell is a red (slow) skeletal muscle cell, a white (fast) skeletal muscle cell or an intermediate skeletal muscle cell.

In some embodiments, methods of modifying described herein comprise contacting cells with compositions or systems described herein. In some embodiments, the contacting comprises electroporation, acoustic poration, optoporation, viral vector-based delivery, iTOP, nanoparticle delivery (e.g., lipid or gold nanoparticle delivery), cell-penetrating peptide (CPP) delivery, DNA nanostructure delivery, or any combination thereof.

In some embodiments, methods of modifying described herein are performed in a subject. In some embodiments, the methods comprise administering compositions described herein to the subject. In some embodiments, the subject is a human. In some embodiments, the subject is a mammal (e.g., rat, mouse, cow, dog, pig, sheep, horse). In some embodiments, the subject is a vertebrate or an invertebrate. In some embodiments, the subject is a laboratory animal. In some embodiments, the subject is a patient. In some embodiments, the subject is at risk of developing, suffering from, or displaying symptoms of a disease. In some embodiments, the subject has a mutation associated with a gene described herein. In some embodiments, the subject displays symptoms associated with a mutation of a gene described herein.

### Illustrative Embodiments

The present disclosure provides the following illustrative embodiments.

Embodiment 1. A system comprising:
(i) a polypeptide, or a recombinant nucleic acid encoding the polypeptide, wherein the polypeptide comprises an amino acid sequence that is at least 85% identical to any one of the sequences set forth in TABLE 1 or TABLE 1.2; and
(ii) an engineered guide nucleic acid or a nucleic acid that encodes the engineered guide nucleic acid.

Embodiment 2. The system of embodiment 1, wherein the polypeptide comprises an amino acid sequence that is at least 85% identical to any one of sequences SEQ ID NO: 1-7, 9, 11-15, 68-73, 76-87, and 94-99 listed in TABLE 1.

Embodiment 3. The system of embodiment 1, wherein the polypeptide comprises an amino acid sequence that is at least 89% identical to SEQ ID NO: 88.

Embodiment 4. The system of embodiment 1, wherein the polypeptide comprises an amino acid sequence that is at least 91% identical to SEQ ID NO: 89.

Embodiment 5. The system of embodiment 1, wherein the polypeptide comprises an amino acid sequence that is at least 92% identical to SEQ ID NO: 90 and 92.

Embodiment 6. The system of embodiment 1, wherein the polypeptide comprises an amino acid sequence that is at least 93% identical to SEQ ID NO: 74-75.

Embodiment 7. The system of embodiment 1, wherein the polypeptide comprises an amino acid sequence that is at least 94% identical to SEQ ID NO: 91.

Embodiment 8. The system of embodiment 1, wherein the polypeptide comprises an amino acid sequence that is at least 98% identical to SEQ ID NO: 8.

Embodiment 9. The system of embodiment 1, wherein the polypeptide comprises an amino acid sequence that is at least 99.5% identical to SEQ ID NO: 10.

Embodiment 10. The system of any one of embodiments 1-9, wherein the polypeptide interacts with an engineered guide nucleic acid.

Embodiment 11. The system of embodiment 10, wherein the engineered guide nucleic acid comprises a repeat sequence and a spacer sequence.

Embodiment 12. The system of embodiment 10, wherein the engineered guide nucleic acid comprises a crRNA.

Embodiment 13. The system of any one of embodiments 1-12, wherein the engineered guide nucleic acid comprises a first region and a second region, wherein the second region comprises a nucleotide sequence that is complementary to a target sequence in a target nucleic acid, wherein the first region and the second region are heterologous to each other.

Embodiment 14. The system of embodiment 13, wherein the first region is covalently linked to the 5′ end of the second region.

Embodiment 15. The system of embodiment 13, wherein the first region comprises a repeat sequence, wherein the repeat sequence is at least 75% identical to any one of nucleotide sequences set forth in TABLE 3.

Embodiment 16. The system of embodiment 13, wherein the engineered guide nucleic acid comprises a repeat sequence and wherein;
(a) the polypeptide comprises an amino acid sequence that is at least 85% identical to SEQ ID NO: 1 and wherein the repeat sequence comprises a nucleotide sequence that is at least 75% identical to SEQ ID NO: 30 or SEQ ID NO: 50;
(b) the polypeptide comprises an amino acid sequence that is at least 85% identical to SEQ ID NO: 2 and wherein the repeat sequence comprises a nucleotide sequence that is at least 75% identical to SEQ ID NO: 31, SEQ ID NO: 35, or SEQ ID NO: 47;
(c) the polypeptide comprises an amino acid sequence that is at least 85% identical to SEQ ID NO: 3 and wherein the repeat sequence comprises a nucleotide sequence that is at least 75% identical to SEQ ID NO: 32 or SEQ ID NO: 39;
(d) the polypeptide comprises an amino acid sequence that is at least 85% identical to SEQ ID NO: 4 and wherein the repeat sequence comprises a nucleotide sequence that is at least 75% identical to SEQ ID NO: 33 or SEQ ID NO: 44;
(e) the polypeptide comprises an amino acid sequence that is at least 85% identical to SEQ ID NO: 5 and wherein the repeat sequence comprises a nucleotide sequence that is at least 75% identical to SEQ ID NO: 34, SEQ ID NO: 40, or SEQ ID NO: 48;
(f) the polypeptide comprises an amino acid sequence that is at least 85% identical to SEQ ID NO: 6 and wherein the repeat sequence comprises a nucleotide sequence that is at least 75% identical to SEQ ID NO: 35 or SEQ ID NO: 31;
(g) the polypeptide comprises an amino acid sequence that is at least 85% identical to SEQ ID NO: 7 and wherein the repeat sequence comprises a nucleotide sequence that is at least 75% identical to SEQ ID NO: 36, SEQ ID NO: 48, or SEQ ID NO: 52;
(h) the polypeptide comprises an amino acid sequence that is at least 85% identical to SEQ ID NO: 8 and wherein the repeat sequence comprises a nucleotide sequence that is at least 75% identical to SEQ ID NO: 37 or SEQ ID NO: 42;
(i) the polypeptide comprises an amino acid sequence that is at least 85% identical to SEQ ID NO: 9 and wherein the repeat sequence comprises a nucleotide sequence that is at least 75% identical to SEQ ID NO: 38 or SEQ ID NO: 58;
(j) the polypeptide comprises an amino acid sequence that is at least 85% identical to SEQ ID NO: 10 and wherein the repeat sequence comprises a nucleotide sequence that is at least 75% identical to SEQ ID NO: 39, SEQ ID NO: 32, or SEQ ID NO: 49;
(k) the polypeptide comprises an amino acid sequence that is at least 85% identical to SEQ ID NO: 11 and wherein the repeat sequence comprises a nucleotide sequence that is at least 75% identical to SEQ ID NO: 40 or SEQ ID NO: 34;
(l) the polypeptide comprises an amino acid sequence that is at least 85% identical to SEQ ID NO: 12 and wherein the repeat sequence comprises a nucleotide sequence that is at least 75% identical to SEQ ID NO: 41 or SEQ ID NO: 46;
(m) the polypeptide comprises an amino acid sequence that is at least 85% identical to SEQ ID NO: 13 and wherein the repeat sequence comprises a nucleotide sequence that is at least 75% identical to SEQ ID NO: 42;
(n) the polypeptide comprises an amino acid sequence that is at least 85% identical to SEQ ID NO: 14 and wherein the repeat sequence comprises a nucleotide sequence that is at least 75% identical to SEQ ID NO: 43 or SEQ ID NO: 30;
(o) the polypeptide comprises an amino acid sequence that is at least 85% identical to SEQ ID NO: 15 and wherein the repeat sequence comprises a nucleotide sequence that is at least 75% identical to SEQ ID NO: 44 or SEQ ID NO: 33;
(p) the polypeptide comprises an amino acid sequence that is at least 85% identical to SEQ ID NO: 68 and wherein the repeat sequence comprises a nucleotide sequence that is at least 75% identical to SEQ ID NO: 48 or SEQ ID NO: 59;
(q) the polypeptide comprises an amino acid sequence that is at least 85% identical to SEQ ID NO: 69 and wherein the repeat sequence comprises a nucleotide sequence that is at least 75% identical to SEQ ID NO: 57 or SEQ ID NO: 60;
(r) the polypeptide comprises an amino acid sequence that is at least 85% identical to SEQ ID NO: 70 and wherein the repeat sequence comprises a nucleotide sequence that is at least 75% identical to SEQ ID NO: 61;
(s) the polypeptide comprises an amino acid sequence that is at least 85% identical to SEQ ID NO: 71 and wherein the repeat sequence comprises a nucleotide sequence that is at least 75% identical to SEQ ID NO: 62;
(t) the polypeptide comprises an amino acid sequence that is at least 85% identical to SEQ ID NO: 72 and wherein the repeat sequence comprises a nucleotide sequence that is at least 75% identical to SEQ ID NO: 63;
(u) the polypeptide comprises an amino acid sequence that is at least 85% identical to SEQ ID NO: 73 and wherein the repeat sequence comprises a nucleotide sequence that is at least 75% identical to SEQ ID NO: 64;
(v) the polypeptide comprises an amino acid sequence that is at least 85% identical to SEQ ID NO: 74 and wherein the repeat sequence comprises a nucleotide sequence that is at least 75% identical to SEQ ID NO: 65;
(w) the polypeptide comprises an amino acid sequence that is at least 85% identical to SEQ ID NO: 75 and wherein the repeat sequence comprises a nucleotide sequence that is at least 75% identical to SEQ ID NO: 66;
(x) the polypeptide comprises an amino acid sequence that is at least 85% identical to SEQ ID NO: 76 and wherein the repeat sequence comprises a nucleotide sequence that is at least 75% identical to SEQ ID NO: 67
(y) the polypeptide comprises an amino acid sequence that is at least 85% identical to SEQ ID NO: 93 and wherein the repeat sequence comprises a nucleotide sequence that is at least 75% identical to SEQ ID NO: 45;
(z) the polypeptide comprises an amino acid sequence that is at least 85% identical to any one of SEQ ID NO: 94-97 and wherein the repeat sequence comprises a nucleotide sequence that is at least 75% identical to SEQ ID NO: 53;
(aa) the polypeptide comprises an amino acid sequence that is at least 85% identical to SEQ ID NO: 97 and wherein the repeat sequence comprises a nucleotide sequence that is at least 75% identical to SEQ ID NO: 54;
(bb) the polypeptide comprises an amino acid sequence that is at least 85% identical to SEQ ID NO: 98 or 99 and wherein the repeat sequence comprises a nucleotide sequence that is at least 75% identical to SEQ ID NO: 55; or
(cc) the polypeptide comprises an amino acid sequence that is at least 85% identical to any one of SEQ ID NO: 90, 91, 92, 98, or 99 and wherein the repeat sequence comprises a nucleotide sequence that is at least 75% identical to SEQ ID NO: 56.

Embodiment 17. The system of any one of embodiments 13-15, wherein the first region, at least partially, interacts with the polypeptide.

Embodiment 18. The system of any one of embodiments 13-14, wherein the second region comprises a spacer sequence.

Embodiment 19. The system of embodiment 18, wherein the spacer sequence is at least 80%, at least 85%, at least 90%, at least 95%, at least 98%, at least 99% or 100% complementary to the target sequence.

Embodiment 20. The system of any one of embodiments 10-16, wherein the engineered guide nucleic acid or a portion thereof hybridizes to a target nucleic acid.

Embodiment 21. The system of any one of embodiments 20, wherein the target nucleic acid is RNA.

Embodiment 22. The system of embodiment 21, wherein the RNA is single stranded RNA, double stranded RNA, linear single-stranded RNA, circular RNA, coding RNA, non-coding RNA, or combinations thereof.

Embodiment 23. The system of any one of embodiments 1-22, wherein the system modifies a target nucleic acid when a complex comprising the polypeptide and an engineered guide nucleic acid hybridizes to a target sequence in a target nucleic acid.

Embodiment 24. The system of embodiment 23, wherein the complex comprising the polypeptide and an engineered guide nucleic acid cleaves the target nucleic acid within the target sequence or within 50 nucleotides of the 5′ or 3′ end of the target sequence.

Embodiment 25. The system of embodiment 23, wherein the complex comprising the polypeptide and an engineered guide nucleic acid cleaves a non-target nucleic acid.

Embodiment 26. The system of embodiment 25, wherein the non-target nucleic acid is selected from a RNA and a ssDNA.

Embodiment 27. The system of embodiment 24 or 25, wherein the engineered guide nucleic acid comprises at least 10 contiguous nucleotides that are complementary to the target sequence in the target nucleic acid.

Embodiment 28. The system of any one of embodiments 24, 25, or 27, wherein the engineered guide nucleic acid comprises one or more phosphorothioate (PS) backbone modifications, 2′-fluoro (2′-F) sugar modifications, or 2′-O-Methyl (2′OMe) sugar modifications.

Embodiment 29. The system of any one of embodiments 23-28, wherein the system comprises an additional engineered guide nucleic acid, at least a portion of which hybridizes to a different target sequence of the target nucleic acid than the engineered guide nucleic acid.

Embodiment 30. The system of any one of embodiments 1-29, wherein the polypeptide is fused to at least one heterologous polypeptide.

Embodiment 31. The system of embodiment 30, wherein a heterologous polypeptide comprises a nuclear localization signal (NLS).

Embodiment 32. The system of any one of embodiments 1-31, wherein the polypeptide comprises a length of about 800 amino acids to about 1,500 amino acids.

Embodiment 33. The system of embodiment 32, wherein the polypeptide comprises a higher eukaryotes and prokaryotes nucleotide (HEPN) domain that is capable of cleaving a target nucleic acid.

Embodiment 34. The system of any one of embodiments 1-33, wherein the polypeptide is capable of cleaving a target nucleic acid.

Embodiment 35. The system of any one of embodiments 1-33, wherein the polypeptide is capable of modifying at least one nucleotide of a target nucleic acid.

Embodiment 36. The system of embodiment 35, wherein modifying comprises cleaving the target nucleic acid, including silencing, degradation, or splicing of at least one nucleotide of the target nucleic acid.

Embodiment 37. The system of embodiment 35, wherein the polypeptide is fused to a base editing enzyme, optionally wherein the base editing enzyme comprises a deaminase.

Embodiment 38. The system of embodiment 36, wherein modifying comprises modifying a nucleobase of at least one nucleotide of the target nucleic acid.

Embodiment 39. A system for detecting a target nucleic acid, comprising the system of any one of embodiments 1-38, and a reporter, wherein the reporter comprises a nucleic acid and a detectable moiety, and wherein the nucleic acid comprises RNA, ssDNA, or a combination thereof.

Embodiment 40. The system of embodiment 39, wherein cleavage of the reporter generates a detectable product or detectable signal from the detectable moiety.

Embodiment 41. The system of embodiment 39, wherein cleavage of the reporter reduces a detectable signal from the detectable moiety.

Embodiment 42. The system of embodiment 39, wherein cleavage of the reporter is effective to produce a detectable product comprising a detectable moiety.

Embodiment 43. The system of any one of embodiments 39-42, wherein the detectable moiety comprises a fluorophore, a quencher, a FRET (fluorescence resonance energy transfer) pair, a fluorescent protein, horseradish peroxidase protein, a colorimetric signal, an antigen or a combination thereof.

Embodiment 44. The system of any one of embodiments 39-42, wherein the reporter comprises a fluorophore which is attached to a quencher by the nucleic acid, and wherein, upon cleavage of the nucleic acid, the fluorophore generates a signal, wherein the signal is detected as a positive signal, indicating the presence of the target nucleic acid.

Embodiment 45. The system of any one of embodiments 39-43, wherein the reporter is configured to generate a signal indicative of a presence or absence of the target nucleic acid.

Embodiment 46. The system of any one of embodiments 39-42, wherein the reporter is cleaved by the polypeptide.

Embodiment 47. The system of any one of embodiments 39-42, wherein the reporter is configured to release a detection moiety when cleaved by the polypeptide following hybridizing of the engineered guide nucleic acid to the target nucleic acid, and wherein release of the detection moiety is indicative of a presence or absence of the target nucleic acid.

Embodiment 48. The system of embodiment 39, comprising at least one detection reagent for detecting a target nucleic acid.

Embodiment 49. The system of embodiment 48, wherein the at least one detection reagent is selected from a reporter nucleic acid, a detection moiety, an additional polypeptide, or a combination thereof, optionally wherein the reporter nucleic acid comprises a fluorophore, a quencher, or combinations thereof.

Embodiment 50. The system of embodiment 48 or 49, wherein the at least one detection reagent is operably linked to a polypeptide, such that a detection event occurs upon contacting the system with a target nucleic acid.

Embodiment 51. The system of any one of embodiments 39-42, 44-47, or 49, wherein the reporter is operably linked to a polypeptide.

Embodiment 52. The system of any one of embodiments 1-51, wherein the engineered guide nucleic acid is capable of hybridizing to a target sequence in a target nucleic acid, and wherein the target nucleic acid is any one of: a naturally occurring eukaryotic sequence, an engineered eukaryotic sequence, a fragment of a naturally occurring eukaryotic sequence, a fragment of an engineered eukaryotic sequence, and combinations thereof.

Embodiment 53. The system of embodiment 52, wherein the target nucleic acid is isolated from a human cell.

Embodiment 54. The system of embodiment 1, wherein the recombinant nucleic acid encoding the polypeptide is a nucleic acid expression vector.

Embodiment 55. The system of embodiment 54, wherein the nucleic acid expression vector is a viral vector.

Embodiment 56. The system of embodiment 55, wherein the nucleic acid expression vector is an adeno associated viral (AAV) vector.

Embodiment 57. The system of any one of embodiments 54-56, wherein the nucleic acid expression vector encodes at least one engineered guide nucleic acid.

Embodiment 58. A system comprising an engineered polypeptide, or a recombinant nucleic acid encoding the engineered polypeptide, wherein the engineered polypeptide comprises an amino acid sequence that is at least 85% identical to any one of the sequences set forth in TABLE 1 or TABLE 1.2.

Embodiment 59. The system of any one of embodiments 1-58, wherein the system is present in a single composition.

Embodiment 60. A pharmaceutical composition, comprising the system of embodiment 59; and a pharmaceutically acceptable excipient, carrier or diluent.

Embodiment 61. A method of detecting a presence of a target nucleic acid in a sample, the method comprising:
(a) contacting the sample with the system of any one of embodiments 1-59;
(b) cleaving a reporter with the polypeptide in response to formation of a complex comprising the polypeptide, an engineered guide nucleic acid, and a target sequence in a target nucleic acid, thereby producing a detectable product; and
(c) detecting the detectable product, thereby detecting the presence of the target nucleic acid in the sample.

Embodiment 62. The method of embodiment 61, wherein the target nucleic acid is double-stranded RNA, single-stranded RNA, linear RNA, circular RNA, coding RNA, non-coding RNA, or combinations thereof.

Embodiment 63. A method of detecting a presence of a target nucleic acid in a sample, the method comprising:
(a) contacting the sample with the system of any one of embodiments 1-59;
(b) cleaving a non-target sequence in a non-target nucleic acid with the polypeptide in response to formation of a complex comprising the polypeptide, an engineered guide nucleic acid, and a target sequence in a target nucleic acid, thereby producing a detectable product; and
(c) detecting the detectable product, thereby detecting the presence of the target nucleic acid in the sample,
wherein the target nucleic acid is amplified DNA, DNA synthesized from a single-stranded RNA template, or cDNA,
wherein the non-target nucleic acid part of a reporter, and
wherein the polypeptide is capable of both hybridizing to the target nucleic acid and cleaving the non-target sequence.

Embodiment 64. The method of embodiment 63, wherein the amplified DNA, DNA synthesized from single-stranded RNA template, or cDNA is double-stranded DNA, single-stranded DNA, or combinations thereof.

Embodiment 65. The method of any one of embodiments 61-64, wherein the target nucleic acid comprises any one of: a naturally occurring eukaryotic sequence, a naturally occurring prokaryotic sequence, a naturally occurring viral sequence, a naturally occurring bacterial sequence, a naturally occurring fungal sequence, an engineered eukaryotic sequence, an engineered prokaryotic sequence, an engineered viral sequence, an engineered bacterial sequence, an engineered fungal sequence, a fragment of a naturally occurring sequence, a fragment of an engineered sequence, and combinations thereof.

Embodiment 66. The method of any one of embodiments 61-65, wherein the target nucleic acid is isolated from any one of: a naturally occurring cell, a eukaryotic cell, a prokaryotic cell, a plant cell, a fungal cell, an animal cell, cell of an invertebrate, a fly cell, a cell of a vertebrate, a mammalian cell, a primate cell, a non-human primate cell, a human cell, a living cell, a non-living cell, a modified cell, a derived cell, and a non-naturally occurring cell.

Embodiment 67. The method of embodiment 61 or 63, wherein the detectable product further comprises a detectable label or a nucleic acid comprising a detectable label selected from a reporter nucleic acid, a detection moiety, an additional polypeptide, or a combination thereof, optionally wherein the reporter nucleic acid comprises a fluorophore, a quencher, or a combination thereof.

Embodiment 68. A method of modifying a target nucleic acid, the method comprising contacting the target nucleic acid with the system of any one of embodiments 1-59, or the pharmaceutical composition of embodiment 60, thereby producing a modified target nucleic acid.

Embodiment 69. The method of embodiment 68, wherein modifying the target nucleic acid comprises splicing, silencing, or degradation of the target nucleic acid or a target sequence in the target nucleic acid.

Embodiment 70. The method of embodiment 69, wherein splicing the target nucleic acid comprises removal or degradation of a sequence of interest, a non-coding region, an intron, an intron fragment, or any combinations thereof.

Embodiment 71. The method of embodiment 69, wherein splicing the target nucleic acid comprises rejoining of a sequence of interest, a coding region, an exon, an exon fragment, or any combinations thereof.

Embodiment 72. The method of any one of embodiments 61-71, wherein the method is performed in in vitro.

Embodiment 73. The method of anyone of embodiments 61-66 or 68-71, wherein the target nucleic acid is associated with any one of the genes recited in TABLE 4 or TABLE 4.1.

Embodiment 74. The method of any one of embodiments 61-66 or 68-71, wherein the target nucleic acid comprises a mutation associated with a disease or disorder.

Embodiment 75. The method of any one of embodiments 61-66 or 68-71, wherein the target nucleic acid comprises one or more mutations.

Embodiment 76. The method of embodiment 75, wherein the one or more mutations comprise a point mutation, a single nucleotide polymorphism (SNP), a missense mutation, a nonsense mutations, a transcription-associated mutation, or any combination thereof.

Embodiment 77. The method of embodiment 74, wherein the disease or disorder is any one of the diseases or disorders recited in TABLE 5 or TABLE 5.1.

Embodiment 78. The method of embodiment 68, wherein the modified target nucleic acid no longer comprises a mutation associated with a disease or disorder as compared to an unmodified target nucleic acid.

Embodiment 79. The method of embodiment 68, wherein the modified target nucleic acid produces a polypeptide having new activity as compared to an unmodified target nucleic acid, or alters expression of an endogenous polypeptide as compared to an unmodified target nucleic acid.

Embodiment 80. A method of treating a disease or disorder associated with a mutation or aberrant expression of a gene in a subject in need thereof, the method comprising administering to the subject the pharmaceutical composition of embodiment 60.

Embodiment 81. A system comprising:
(a) a polypeptide, or a nucleic acid encoding the polypeptide, and an engineered guide nucleic acid, or a nucleic acid that encodes the engineered guide nucleic acid;
(b) a polypeptide, or a nucleic acid encoding the polypeptide, and an engineered guide nucleic acid comprising a crRNA;
(c) an mRNA encoding a polypeptide, and an engineered guide nucleic acid;
(d) one or more recombinant expression vectors comprising: i) a nucleic acid encoding a polypeptide; and ii) an engineered guide nucleic acid; or
(e) one or more recombinant expression vectors comprising: i) a nucleic acid encoding a polypeptide; and ii) an engineered guide nucleic acid comprising a crRNA; wherein the polypeptide comprises an amino acid sequence that is at least 85% identical to any one of the sequences set forth in TABLE 1 or TABLE 1.2.

Embodiment 82. A kit comprising:
(a) a polypeptide, or a nucleic acid encoding the polypeptide, and an engineered guide nucleic acid, or a nucleic acid that encodes the engineered guide nucleic acid;
(b) a polypeptide, or a nucleic acid encoding the polypeptide, and an engineered guide nucleic acid comprising a crRNA;
(c) an mRNA encoding a polypeptide, and an engineered guide nucleic acid;
(d) one or more recombinant expression vectors comprising: i) a nucleic acid encoding a polypeptide; and ii) an engineered guide nucleic acid; or
(e) one or more recombinant expression vectors comprising: i) a nucleic acid encoding a polypeptide; and ii) an engineered guide nucleic acid comprising a crRNA; wherein the polypeptide comprises an amino acid sequence that is at least 85% identical to any one of the sequences set forth in TABLE 1 or TABLE 1.2.

Embodiment 83. The kit of embodiment 82, wherein components of the kit are in same container.

Embodiment 84. The kit of embodiment 82, wherein components of the kit are in separate containers.

Embodiment 85. A container comprising:
(a) a polypeptide, or a nucleic acid encoding the polypeptide, and an engineered guide nucleic acid, or a nucleic acid that encodes the engineered guide nucleic acid;
(b) a polypeptide, or a nucleic acid encoding the polypeptide, and an engineered guide nucleic acid comprising a crRNA;
(c) an mRNA encoding a polypeptide, and an engineered guide nucleic acid;
(d) one or more recombinant expression vectors comprising: i) a nucleic acid encoding a polypeptide; and ii) an engineered guide nucleic acid; or
(e) one or more recombinant expression vectors comprising: i) a nucleic acid encoding a polypeptide; and ii) an engineered guide nucleic acid comprising a crRNA; wherein the polypeptide comprises an amino acid sequence that is at least 85% identical to any one of the sequences set forth in TABLE 1 or TABLE 1.2.

Embodiment 86. The container of embodiment 85, wherein the container is a syringe.

Embodiment 87. A device comprising:
(a) a polypeptide, or a nucleic acid encoding the polypeptide, and an engineered guide nucleic acid, or a nucleic acid that encodes the engineered guide nucleic acid;
(b) a polypeptide, or a nucleic acid encoding the polypeptide, and an engineered guide nucleic acid comprising a crRNA;
(c) an mRNA encoding a polypeptide, and an engineered guide nucleic acid;
(d) one or more recombinant expression vectors comprising: i) a nucleic acid encoding a polypeptide; and ii) an engineered guide nucleic acid; or
(e) one or more recombinant expression vectors comprising: i) a nucleic acid encoding a polypeptide; and ii) an engineered guide nucleic acid comprising a crRNA; wherein the polypeptide comprises an amino acid sequence that is at least 85% identical to any one of the sequences set forth in TABLE 1 or TABLE 1.2.

Embodiment 88. The device of embodiment 87, wherein the device is used in diagnosis of a disease or disorder associated with a nucleic acid sequence modification in a viral genome, a prokaryotic genome, or a eukaryotic genome.

Embodiment 89. The device of embodiment 87, wherein the device is used in diagnosis of a disease or disorder associated with a non-wild type gene, a gene comprising a non-wild type reading frame, a gene comprising one or more mutations, abnormal processing upon transcription of a gene, combinations thereof.

Embodiment 90. A microfluidic device comprising:
(a) a sample interface configured to receive a sample comprising nucleic acids; and
(b) a chamber fluidically connected to the sample interface;
wherein the chamber comprises a polypeptide and an engineered guide nucleic acid, wherein the polypeptide comprises an amino acid sequence that is at least 85% identical to any one of the sequences set forth in TABLE 1 or TABLE 1.2.

Embodiment 91. The microfluidic device of embodiment 90, wherein the chamber further comprises a reporter comprising a nucleic acid and a detection moiety.

Embodiment 92. The microfluidic device of embodiment 91, wherein the polypeptide is effective to form an activated complex with the engineered guide nucleic acid upon hybridization of the engineered guide nucleic acid to a target sequence of a target nucleic acid and wherein the nucleic acid of the reporter is a cleavage substrate of the activated complex.

Embodiment 93. The microfluidic device of embodiment 91, wherein the reporter is immobilized to a surface within the chamber.

Embodiment 94. The microfluidic device of embodiment 91, wherein nucleic acid of the reporter comprises a ribonucleotide, a deoxyribonucleotide, or combinations thereof.

Embodiment 95. The microfluidic device of any one of embodiments 90-94, further comprising a valve disposed between the sample interface and the chamber, optionally wherein the valve is configured to selectively resist flow, or permit flow.

Embodiment 96. The microfluidic device of any one of embodiments 90-95, wherein the chamber further comprises one or more reagents for amplification, one or more cell lysis reagents, one or more nucleic acid purification reagents.

Embodiment 97. The microfluidic device of any one of embodiments 90-96, wherein the chamber further comprises a polymerase, a reverse transcriptase, or a combination thereof.

Embodiment 98. The microfluidic device of any one of embodiments 90-97, wherein the chamber is a first chamber and the microfluidic device further comprising a second chamber comprising one or more reagents for amplification, one or more cell lysis reagents, one or more nucleic acid purification reagents.

Embodiment 99. The microfluidic device of any one of embodiments 90-98, further comprising a channel comprising one or more reagents for amplification, one or more cell lysis reagents, one or more nucleic acid purification reagents.

Embodiment 100. The microfluidic device of embodiment 98 or 99, wherein the second chamber or channel is disposed between the sample interface and the first chamber, wherein the second chamber or channel is disposed downstream of the sample interface and the first chamber, wherein the second chamber or channel is disposed upstream of the sample interface and the first chamber.

Embodiment 101. The microfluidic device of any one of embodiments 90-100, comprising a plurality of chambers fluidically connected to a plurality of valves.

Embodiment 102. The microfluidic device of embodiment 101, wherein a first subset of the plurality of valves are configured to restrict flow in a first direction through one or more channels towards the sample interface.

Embodiment 103. The microfluidic device of embodiment 101, wherein a second subset of the plurality of valves are configured to selectively permit flow in a second direction through one or more channels towards a reaction chamber.

Embodiment 104. The microfluidic device of embodiment 101, wherein a first valve and a second valve of the plurality of valves are configured to physically, fluidically, or thermally isolate a first portion of the sample from a second portion of the sample when the first valve and the second valve are in a closed state.

Embodiment 105. The microfluidic device of any one of embodiments 90-104, wherein each valve of the plurality of valves comprises a valve inlet channel and a valve outlet channel; and wherein a cross-sectional area of the valve inlet channel is less than a cross-sectional area of the corresponding valve outlet channel.

Embodiment 106. The microfluidic device of any one of embodiments 90-105, wherein each valve is thermally connected to a heating element.

Embodiment 107. The microfluidic device of any one of embodiments 90-106, wherein each valve is filled with a material configured to change between liquid and solid phases when heated by a heating element.

Embodiment 108. The microfluidic device of any one of embodiments 90-107, further comprising a detection region fluidically connected to the first chamber.

Embodiment 109. The microfluidic device of embodiment 108, wherein the detection region comprises an array, one or more lateral flow strips, a detection tray, a detection region comprising a capture antibody, or combinations thereof.

Embodiment 110. The system of any one of embodiments 1-59 or 81, the kit of any one of embodiments 82-84, the device of any one of embodiments 87-89, or the microfluidic device of any one of embodiments 90-109, wherein components of the system, kit, device, or microfluidic device are used in diagnosis of a disease or disorder.

Embodiment 111. The system of any one of embodiments 1-59 or 81, the kit of any one of embodiments 82-84, the device of any one of embodiments 87-89, or the microfluidic device of any one of embodiments 90-109, wherein components of the system, kit, device, or microfluidic device are used in diagnosis of a disease or disorder associated with a nucleic acid sequence modification in a disease or disorder associated gene selected from a viral genome, a prokaryotic genome, or an eukaryotic genome.

Embodiment 112. The system of any one of embodiments 1-59 or 81, the kit of any one of embodiments 82-84, the device of any one of embodiments 87-89, or the microfluidic device of any one of embodiments 90-109, wherein components of the system, kit, device, or microfluidic device are used in diagnosis of a disease or disorder associated with a non-wild type gene, a gene comprising a non-wild type reading frame; a gene comprising one or more mutations, or abnormal processing upon transcription of a gene.

Embodiment 113. A method for diagnosis comprising the use of the system of any one of embodiments 1-59 or 81, the kit of any one of embodiments 82-84, the device of any one of embodiments 87-89, or the microfluidic device of any one of embodiments 90-109, wherein components of the system, kit, device, or microfluidic device further comprises a detectable label or a nucleic acid comprising a detectable label capable of hybridizing to a target nucleic acid.

Embodiment 114. The method of diagnosis of embodiment 113, wherein hybridizing to a target nucleic acid results in modification of a detectable label, and wherein the detectable label emits a detectable signal upon modification.

Embodiment 115. The method of embodiment 114, wherein the target nucleic acid is in one or more of: a naturally occurring cell, a eukaryotic cell, a prokaryotic cell, a plant cell, a fungal cell, an animal cell, cell of an invertebrate, a fly cell, a cell of a vertebrate, a mammalian cell, a primate cell, a non-human primate cell, a human cell, a living cell, a non-living cell, a modified cell, a derived cell, and a non-naturally occurring cell.

Embodiment 116. A composition comprising:
(a) a polypeptide, or a nucleic acid encoding the polypeptide, and an engineered guide nucleic acid, or a nucleic acid that encodes the engineered guide nucleic acid;
(b) a polypeptide, or a nucleic acid encoding the polypeptide, and an engineered guide nucleic acid comprising a crRNA;
(c) an mRNA encoding a polypeptide, and an engineered guide nucleic acid;
(d) one or more recombinant expression vectors comprising: i) a nucleic acid encoding a polypeptide; and ii) an engineered guide nucleic acid; or
(e) one or more recombinant expression vectors comprising: i) a nucleic acid encoding a polypeptide; and ii) an engineered guide nucleic acid comprising a crRNA;
wherein the polypeptide comprises an amino acid sequence that is at least 85% identical to any one of the sequences set forth in TABLE 1 or TABLE 1.2.

Embodiment 117. A system comprising a polypeptide, or a recombinant nucleic acid encoding the polypeptide, wherein the polypeptide comprises a variant amino acid sequence of SEQ ID NO: 69, or a functional fragment thereof, wherein the variant amino acid sequence comprises one or more amino acid alterations at one or more residues corresponding to one or more positions listed in TABLE 1.1; and optionally wherein the amino acid sequence, other than the one or more amino acid alterations, has at least 85% sequence identity to the amino acid sequence referenced in SEQ ID NO: 69.

Embodiment 118. The system of embodiment 117, wherein the one or more amino acid alterations are individually at one or more residues corresponding to one or more positions selected from: 9, 15, 56, 106, 121, 125, 131, 139, 150, 154, 164, 166, 175, 184, 198, 200, 242, 247, 262, 265, 281, 289, 305, 311, 313, 314, 318, 333, 338, 352, 372, 381, 480, 485, 492, 496, 501, 517, 521, 537, 543, 546, 547, 548, 555, 559, 567, 569, 574, 579, 585, 618, 621, 622, 623, 631, 647, 656, 684, 705, 709, 717, 722, 726, 737, 747, 762, 765, 766, 769, 789, 790, 800, 801, 807, 819, 827, 836, 843, 846, 847, 857, 858, 864, 867, 870, 871, 909, 915, 919, 923, 927, 974, 1011, 1020, 1030, 1032, 1035, 1049, 1054, 1056, 1062, 1064, 1083, 1085, or combinations thereof, relative to SEQ ID NO: 69.

Embodiment 119. The system of embodiment 117, wherein the one or more amino acid alterations are individually at one or more residues corresponding to one or more positions selected from: 121, 139, 311, 184, 154, 547, 318, 656, 372, 858, 548, 352, 927, 737, 1062, 819, 501, 974, 1064, 722, 621, 765, 622, 807, 762, 871, 800, 827, 1020, or combinations thereof, relative to SEQ ID NO: 69.

Embodiment 120. The system of embodiment 117, wherein the one or more amino acid alterations are each a substitution of an amino acid residue with a basic (positively charged) amino acid, an acidic (negatively charged) amino acid, a non-polar (hydrophobic) amino acid, an uncharged polar amino acid, or combinations thereof.

Embodiment 121. The system of embodiment 117, wherein the one or more amino acid alterations are each a substitution of an amino acid residue with an amino acid residue selected from a group comprising: Gly (G), Lys (K), Ala (A), Gln (Q), Asn (N), Leu (L), Tyr (Y), Arg (R), Glu (E), Met (M), Thr (T), Val (V), Ser (S), His (H), Ile (I), Cys (C), Pro (P), Asp (D), or combinations thereof.

Embodiment 122. The system of embodiment 117, wherein the one or more amino acid alterations are each a substitution of an amino acid residue with an amino acid residue selected from a group comprising: Asn (N), Gln (Q), Val (V), Glu (E), Lys (K), Leu (L), Ala (A), Cys (C), Ile (I), Ser (S), Pro (P), Thr (T), Tyr (Y), Arg (R), Gly (G), or combinations thereof.

Embodiment 123. The system of embodiment 117, wherein each of the one or more amino acid alterations are individually selected from a group comprising: T9G, T15K, Q56A, H106Q, E121N, C125L, E131K, H139Q, N150Y, G154K, H164R, Q166K, Q175K, D184E, E198K, F200Y, A242M, R247T, A262T, N265R, N281K, D289T, H305K, M311V, A313S, N314K, K318Q, E333H, L338I, S352C, V372L, L381M, Q480Y, Q485S, V492Q, H496S, V501T, G517N, S521A, L537R, E543L, W546Y, S547N, G548A, I555L, Y559N, N567S, D569H, D574Q, L579V, Q585L, Q618E, W621Q, I622N, I622K, M623L, D631C, L647V, M656L, L684K, A705T, Q709H, K717M, N722R, T726Q, A737S, T747L, A762G, W765R, W765N, Q766M, K769E, T789Q, N790K, D800K, D800G, D800R, E801M, E801G, S807T, S807R, S819K, S827R, S827K, N836P, N843S, A846P, T847K, E857M, Y858L, E864Q, E867A, D870E, N871K, N909D, N909E, E915V, S919Q, S919K, I923L, M927I, D974Y, L1011V, H1020R, A1030S, T1032V, D1035M, D1049G, Q1054T, Q1056L, S1062P, I1064K, P1083Q, A1085E, or combinations thereof, relative to SEQ ID NO: 69.

Embodiment 124. The system of embodiment 117, wherein each of the one or more amino acid alterations are individually selected from a group comprising: E121N, H139Q, M311V, D184E, G154K, S547N, K318Q, M656L, V372L, Y858L, G548A, S352C, M927I, A737S, S1062P, S819K, V501T, D974Y, I1064K, N722R, W621Q, W765N, I622N, S807R, A762G, N871K, D800R, S827K, H1020R, or combinations thereof, relative to SEQ ID NO: 69.

Embodiment 125. The system of embodiment 117, wherein the one or more amino acid alterations comprise:
(a) E121N, M311V, S547N, M656L, Y858L, M927I, and S1062P relative to SEQ ID NO: 69;
(b) H1390, D184E, K318Q, V372L, G548A, and A737S relative to SEQ ID NO: 69;
(c) H139Q, D184E, K318Q, V372L, G548A, A737S, S819K, D974Y, and I1064K relative to SEQ ID NO: 69;
(d) H139Q, D184E, M311V, K318Q, S352C, V372L, V501T, G548A, N722R, A737S, W765N, S807R, S819K, N871K, D974Y, and I1064K relative to SEQ ID NO: 69;
(e) H139Q, G154K, D184E, M311V, K318Q, S352C, V372L, V501T, G548A, I622N, N722R, A737S, W765N, S807R, S819K, S827K, N871K, D974Y, H1020R, and I1064K relative to SEQ ID NO: 69; or
(f) H139Q, D184E, M311V, K318Q, S352C, V372L, V501T, G548A, W621Q, I622N, N722R, A737S, A762G, W765N, D800R, S807R, S819K, N871K, D974Y, and I1064K relative to SEQ ID NO: 69.

Embodiment 126. A system comprising a polypeptide, or a recombinant nucleic acid encoding the polypeptide, wherein the polypeptide comprises an amino acid sequence that is at least 85% identical to any one of SEQ ID NO: 119-282 listed in TABLE 1.2.

Embodiment 127. The system of embodiment 117 or 126, wherein the system further comprises an engineered guide nucleic acid or a nucleic acid that encodes the engineered guide nucleic acid.

Embodiment 128. A polypeptide, or a recombinant nucleic acid encoding the polypeptide, wherein the polypeptide comprises a variant amino acid sequence of SEQ ID NO: 69, or a functional fragment thereof, wherein the variant amino acid sequence comprises one or more amino acid alterations at one or more residues corresponding to one or more positions listed in TABLE 1.1; and optionally wherein the amino acid sequence, other than the one or more amino acid alterations, has at least 85% sequence identity to the amino acid sequence referenced in SEQ ID NO: 69.

Embodiment 129. A polypeptide, or a recombinant nucleic acid encoding the polypeptide, wherein the polypeptide comprises an amino acid sequence that is at least 85% identical to any one of SEQ ID NO: 119-282 listed in TABLE 1.2.

Embodiment 130. The polypeptide of embodiment 128 or 129, wherein the polypeptide is complexed with and/or interacts with a guide nucleic acid or an engineered guide nucleic acid.

Embodiment 131. A recombinant nucleic acid encoding the polypeptide of embodiment 128 or 129.

### SEQUENCES AND TABLES

TABLE 1 provides illustrative amino acid sequences of effector proteins that are useful in the compositions, systems and methods described herein.

TABLE 1
Exemplary Amino Acid Sequence(s) of Effector Protein(s)

| | SEQ ID | |
|---|---|---|
| ID | NO. | Effector Protein Sequence |
| 2712211 | 1 | MSHNKKSEIKRIGVKSAFSHGNSRLTITRFGRGNDAEVAVDTDSHGSSLS |
| | | FPHRVEKSFTVRKIDDEIDVERSSLAASILNPAETVPSDYLGLKSTLEKEFF |
| | | GEEFPKDSVRIQIIHNILDIQKILGIYINDVLYSIANLQNKEKVEEILGKYLS |
| | | DKKLNNILPSLRPYMGFLGAGFHSYIKSKSYDDSQNKDADEYNKSVLRI |
| | | LSAIRNDLAHYKKDSLLFSQKLCTGIQDIKKNDWKIINEHYTQMINRINH |
| | | SFASNSKTNLIILFKIFSTSRSEQQEMVNEYYRFSILKDGKNLGINMRKLR |
| | | ELITDSYFPEIKTTNYDSIRHKIYTILDFVLYKIVRISNELPSIIQELRDSQNT |
| | | EAKDDLYKKSAWIYGPRLKSQIGNFFEKIRNEISVYTTEELEIEMDSSKFL |
| | | KDDAEPLVKLLAFLCNFWDGKEVNEILSAYVHKFESIQSFLDTLHDLND |
| | | EVVFTSKYDAFNARPENTAYKFAGRAAEQLRVLASIGKMKPDLSGARR |
| | | ALFRDAIEILGIVENSDWLNEDGTFREDMLDQYVLPGNTVSDEEKKKINP |
| | | FRNFIANNVIESRRFMYLARYSKPKSVRALMRRAEIIKYVLARLPEKQID |
| | | TYYTNISDGNDASTFKKIAALTTLLTDFSFTSLYENREAIVENASLSTLNK |
| | | NVKIERLKALTGLYLTVAFVAVKNLVKANARYFIAFSARERDYAQMLE |
| | | KDPTLSSLCLFPEGRDPENCYALTEYYLKKEQENDYHPAPGEPFDKEAC |
| | | RKHLDSIHRHFTKKWRDIFRKEIDEALDIHPTGLLGKAVRNQVEHLNILT |
| | | SALPKYIDDFARNPERTKKWRMTSYFKLYHYLLQRILCDAEELNIPQEW |
| | | KSRILEKGLPSTDLIHACFVSLGYNLPRYKNLTTEALFDEDSESAKEGKK |
| | | A |
| 2711416 | 2 | MKGRIASLRDGMSGIVAYGMIKSDIDGSFYFFHKSHLIGCRITEMEKNMA |
| | | VSFEIRDSQLKPGSKEAYNVALWKDPSAGAIETDKHLEKGIISSFKKNDR |
| | | NFIFGGMIRSESGSNYFFHRSSVVKGNINAMHEGDKVEFLIGKPEGYDYE |
| | | QALEVRDLSRSDAAMEERNGEDWRRIERRAQDDEEKREQLLQTIRAEKP |
| | | ETEKRNQSRAKAAGLKSTFTISEHELVMTSFGRGNRAVPEKRIVEGRVE |
| | | DITAEPVLKVEPRQEQFRIHGRLPEDAYADNPLWISKENPEPQRAGSDLIE |
| | | CRKQLEKMYFGRNFDDNIHIQLIYNILDIDKILSVHINNIVYELNNLRREE |
| | | KADFDDLIGSMRLGQEYDVFMNPAADSELAREKKEVFLKTMGQKQLSY |
| | | FGTLLRDDGLRKINDADERARRREAKQKDSYYFLCLLGMARQSLAHGA |
| | | KKTRAAIYKLDAAFDTENNTARKEARAALDRLYQRKIRSLNANYIERAK |
| | | ADLNILFIAYGVHDDAKKMEIAGEYYDFIVRKEYKNLGFSIRKLRETIEM |
| | | YHAPELKDTTYDTMRAKLNHIFDFTVFDYYRRNECFGNAMVERLRASL |
| | | TEAEKEMNYRVEAEELWNEIGARFINSILPRINKDVIKETKDDKHFSVSL |
| | | EKCDLIGTNATEFSKLIYLFTCFLDGKEINDLLTTMISKFESIEAFIETMDA |
| | | LGLDHTFCADYTLFNESGRIAKELRVINSFARMLDKSDNARPIMYQEAA |
| | | ELLGFPESEDILKRIDEILDKTGSTVKDRKTGFRNFIANNVIESGRFHYLV |
| | | RYGNVKKLRKLANNRKVIDFVLKDIPDAQILRYYNSVYGKHDPMIRDD |
| | | MRAALADTITGISFEQFANVHQDDGGTSEEKADKERKKAAVRVYLTVL |
| | | YLLTKNLVYVNSRYFLAFHCVERDALITDPKRYANKLIRKDFAAFGKTY |
| | | MPFAKEYVESHNKNWRAQAYLRQDMENSDALMVSLFRNSTEHLNAIR |
| | | NAERYIDDIAGFSSYFELYHYLMQRTVIDQYERLLGQGKIEEPVNPKMLE |
| | | YFESVQKHHTYCKDMVKALNVAFAYNLPRYKSLSINGRFDRNRPDVKG |
| | | KGEKTVDEA |
| 2711260 | 3 | MKKKQNAKAIREKLQGQKSEDIRRKEKERQDAIAAERAAEEQRKLELK |
| | | AQAERDRRARMLRAIKVEKPETEKQNQSRAKAAGLKSTFTISERELVMT |
| | | SFGRGNRAVPEKRIVEGHVEDIAIEPTLKVEPQQEQFRIHGRLPEDAYAG |
| | | NPLWIGKENPEPQRNGSDLIGCRKQLEHMYFGRNFEDNIHIQLIYNILDID |
| | | KILSVHINNIVYELNNIMRREDADYDDLIGYLDLDTRYEVFMNPDAHYD |
| | | EKKGRLKTARELKDLFLKMMDQPQVSYFGTVLRDDGLRKIKDRGERAQ |
| | | RMEEKLKDSYYLLCMIGMARQAMAHGDGNTRAAIYKLDRAFDTKNNA |
| | | ARVEARAALDQLYEKKIRALNANFLERAKADLIILFRAFGVNDDATKMK |
| | | IVGEYYDFIVRKEYKNLGFSIRKLRETIEMYQAPELKDMKYDTLRAKLN |
| | | HIFDFTVFDYYRRNECIAEAMVERLRASLTEAEKEMNYRIEAERLWKEIG |
| | | ERFMNDVLPRIDGNVIKSIREDKDFSVSLDKCDLIGADATEFSKLIYLLTC |
| | | FLDGKEINDLLTTLISKFESIEAFLDTMDALGIDHTMAADYTLFNESGRIA |
| | | KELREINSFARMLDKSDSARQIMYVEAAELLGFPESEDILKRIEEILDKKG |
| | | STKWDRKTGFRNFIANNVIESGRFHYLVRYGNAKKLRRLANNRKVIDFV |
| | | LKDIPDAQILRYYNSVFGKREPMIRDGMRSALAEAITGISFEQFANVHQD |
| | | KDGTSEQIADKERKKAIVRVYLTVLYLLTKNLVYVNSRYFLAFHCAERD |
| | | AIVADPDKYLDEKQNVDYYGFAKEFVENHGKNRRAQRYLQVNFANSD |
| | | EQTVRFFRNCTAHLNAIRNADNYIDDIADENSYFELYHYLMQRTVIDQY |
| | | ERLRGMGKIKTPVNPKMIGYFELVSKYHTYCKDMVKALNVGFAYNLPR |
| | | YKNLSIDALFDRNHPGEKGTGKTPVVEE |
| 2712493 | 4 | MKGKVSKKAQREAARNSIISKYSAIDQEREKQRKAEEAIRRENEAARLK |
| | | EAEVAWRLQREPMRYSRQETGAQNQVEELLRAAALKDRLASEVEKSDK |
| | | KPSRAKAAGLKSTLVISDTELLMTSFGKGNTAIAEKRITEAGIEAIPEVPA |
| | | FEVQAQDERFRISGRMVHDAQTDNPLSADKRPGQDLIGCRDQLERLFFG |
| | | ANYEDNVHVQIAYQILDVDKILAIHINNIIYEINNLTRQEGAIDLFEKLGF |
| | | DTTYDKFSTKTNETIETFNKLLRAPQLAYFGDILYDADLIKRLKKERNEQ |
| | | RKARLLAQEKAKQREAYDLLSMLGQVRHSMAHFNPMIYAMDQQFDAQ |
| | | YTKKGDHFDPAQRLASRAALGQLYAGRVNELNAHFLDRAKVDLQILFA |
| | | VEKAVLPEDKKRIVRNYYDFIVRKDYKNQGFSIKLLREMMLTFPEASGL |
| | | LGKTYDSMRARLYRSFDFVVFEHYRQHEQEIDSLVAQLRACCDEKDKQ |
| | | AIYRTEAARVWPLVAPTIMNELLPWMDGQKLAVASSSAMDHTITYDMI |
| | | KDIAIKPDAACFTKIIYLMTRFLDGKEINDLLTTLINKFENIAGFLAILQEK |
| | | KIPCSFAQGYDMFGRSAIIAQELRAVNNFARMSEESPEAKRILFVEAAKV |
| | | LGYPGDDETLGKYIDDMLTAKGEFVARGKGFRNFIANNVIESDRFKYLV |
| | | RYGNVSQVRRLADNERVLAFVLKDIPDTQIVRYYNSCTGSSRSNCTPDM |
| | | RQGLAKLMTGISFERFAEVKQSNRATPAEKDDKVRKQAIIRLYLTVLYL |
| | | LVKNLVYVNSRYFLAFHCVERDSIIYDPERFTPKNIREHYCDFAIERMEG |
| | | GAWNKRVTQYLRQNMANANSWATRAFRNNVEHLNTVRLASSYIADAG |
| | | AFASYFELYHYLMQRRLMEEYASDKAEIEARVANGEPREKYAWFPMEN |
| | | EKILTYFGLVKRYHTYCKDMVKALNVPFAYNLPRYKNLSINELFDRNHY |
| | | LEAKGRGTALATAEAAAQDAGNQ |
| 2712234 | 5 | MAESKKTNAKRMGVKAVLAHGTDKLTMTTFGKGNRAEIAFSEGNKGQ |
| | | GLQTPFPIGKKRFSVQNIDEEIYSLGKSDGKLLETRMPNPAGNVGEDYLK |
| | | FKDILEKQFFGKSFPNDNIRIQIIYLILDIQKILGMNVFDILYTLGNLQDEDI |
| | | DQFGQSLATDSAVSKVVKTMRPYFGLFGGVFQIPPKDKPEQGMEHNINI |
| | | LRCISSLRNATAHNQEKEFPWFKKADYSKIFRNGKGEWDIIESLYRKKIR |
| | | RVNDNFAKNSKVNLDILFALLHASEAQKPEITKDYYHFSILKDGKNLGIN |
| | | LVKVRENIIDTFFRQLRNKRYDSQRQKINVIADFLIFRALNSDREFVENAV |
| | | SRLRLTEDEKAKEKLYRDFARDVWGRVKTNLEPYFRDPKAYILKYKSN |
| | | GKDYVPEKWFANSKLPETGGNSFVKVLSFLCNFLEGKEINEFLTAYIHKF |
| | | ECIQSFIDTIHELNEEIVFTDRYALFNTKDFAKNVATELRVLASIGKMKPD |
| | | LEEAKRLLYKYAIQTLGFPVGMEKYVSDEWLEENVLLNAEDRKNRAKK |
| | | AKVNPFRNFIAGNVIESRRFMYLVRYSKPSTVRALMQNQKIVNYVLHRL |
| | | PPDQVKSYAQVFPQKFHETEATILYLTEQLAQFSFETLIREKQAILDNSGQ |
| | | RRASESIERLKGITGLYLTVAYIAIKNIVKANARYYIAFSIFERDLRLLTEK |
| | | AKIEKVTLEPPIVINEKKTIPNPFTLTQYYLDRDNEIKFPGDPRNKEAFFKF |
| | | LRENKKHFSRQWREWLKEKIMEAKAVQNTGMLLTEARNQVEHLNVLR |
| | | WMSKYISDFRQGTQPMSSYFELFHYLIQRLLLENPDLNLAPYRDWILRSG |
| | | RPDRELIQYTFVSLAYNLARYRNLTKEHHFDEVVLESLREEKQENPER |
| 2715166 | 6 | MAKKMNPKKLREAEKQRKANQFKVEEEKRKKEREEEEARRVIEEEQRR |
| | | KAARIASIEKKDPELAKVKKSKVKAAGLKSTFILGDDRLFMTSFGNGAE |
| | | AKPEKYVIGDTVSDAASKTALNVLSDKNEFHVSGRIIEGARATNPLCTEN |
| | | GKVSYRDDQIHLRSKLEKMYFGHECEDNIHIQLIYQILDIEKILAVSANNI |
| | | VFTVDNMLRRSDSEHDDLIGYMMGQNYNAYKNANQANKYQLFRELLD |
| | | KPQLAYFGKTLLPLDVQGKKIKKDTPLWEEYEKRCYYILAALGMVRQV |
| | | TAHGAEKTRSSMYQLENLSSNSSLYYREMAEVLDQLYSDRVNELNKGF |
| | | LQKAYKDICIINKILGITDPEKQAETVREYYSFIVLKDYKYLGFSIKKLRE |
| | | YITKNHAEQIAKREYDSVRQKLNHILDFFLYRYYDAPENENILDNLVDKL |
| | | RSCTKDSEKALIYEDEAAGIWEVMGDMIETRLLPKMDGDYIGSMSEGAA |
| | | DDSLLADIYVKSKGLRFSEMMYVLTIFLDGKEINDLLTQLINRFDNIASFI |
| | | DVMKHEGINSTFTSKYSIFEKSREISKELRVINSFARMTESSPNVKPIMVIE |
| | | AAKILGYEDDEEQLQAYVQEMMDKEKGPKNAYGKKDNNFRNFIINNVI |
| | | ESSRFKYLVRYGNPNKLRALASNKKVVDFVLKGIPDEQIMAYYNSCNG |
| | | VEKAFFPEMRDNLAEKITGLSFMEFSNVYQGKTQDQRLIVDKERKKNIV |
| | | RIYLTVLYLLLKNLVYVNSRYYMAFHCTERDRGIYDKDKYSSSANERN |
| | | WSLFAKDYLEAYDWETIKNRRYKQITMNFANADSWSIIAFRNCAEHLTV |
| | | IRNADEFIEGIAGFESYFELYHYLVQRHLISQFEFECNTDSTRYPGTKIMST |
| | | ERAEGKLLDYFAKVQKYHTYCKDFVKALCTPFAYNLARYKNLAINELF |
| | | DRNNYLPSKGKTVEPEE |
| 2712237 | 7 | MAKKKVSLREQREAKKNASLAGYRQKSQEQEQKKKEEAAKIEAQKKA |
| | | AMGPQTAGSTQDADMVLRQRRAKEEKEQRAREGVKRHYAKAAGVKS |
| | | VYHIRKGELLMTAFGKNAQALPEKKVAGTQIETLADPSAYSAQINEKAF |
| | | DVVGRLGISARVDNPAYSSKAPGENVVGLKREFEELIFGSHYEDNIHIQIA |
| | | HNIFKIEKTLSVHINNIVYELDNMLRREDDEHDDLIGYLGLGKKWQTFR |
| | | NPMPGKEDTYKLFCELLKKPQLSFFGNILYDEELLKQAQKARKAYEKEK |
| | | AEKKLEQKRENAYDLLCLLGATRQALAHGEASTRSVLYTLDAAYDDEA |
| | | GKLNGSWVNKNQRLASRNALDRLYADQVRRLNKDFLEHSKVDLQILFD |
| | | LYGCKDAAEKREIVRAYYDFVVRKEFKNQGFSFTRLRENLLLQKGASVL |
| | | KEQKYDTMRGRLYRLFDFVLFRYYELHAEESAKLVDKLRATLDETQKE |
| | | QIYEQEAQRVWAMVKKSVIDHLAQNVSVEYITGHQNADPDVNVQMLA |
| | | GVALAEHANAFSELMYLLTCFQDGKEINDLLTSLINRFDNIAGQCALLRE |
| | | RGLECTFAPTYRMFAESEKISRELRAINSFARMDMHVEPALKREMFIEAA |
| | | RVLGYPGSDEELLAYADSMLSKDGELVKTTGRKDFRNFIMSNVIASDRF |
| | | QYIARYCNVNKIRALAANRKVVDFVLKHIPDTQIIRYYNSCEGTGFVELD |
| | | PRMRDYLAHRITGLSFSDFETVQINERFITSDEKAEKAKKQAIISLYLTVL |
| | | YLCVKNLVYVNSRYTIAFHFLEHDSITQDKDYRKSLESKGDLRDYAAAR |
| | | IPVKYADKPEKIAALQFIPEERRVPTRETRQREYLLQNLANSDKWAVRTF |
| | | RNCVQHLNAIRNADSYIGEIDRFDSWFELYHYLVQRSLMDQYRWDSTQ |
| | | KKIISDSEMTGKTLEYFDEIRTYHTYSKDFVKALCVPFGYNLSRFKNLSIN |
| | | ELFDKNNYPLK |
| 2208675 | 8 | MQTNYLGKRIEYNYLKTADKHYFGGFLNLAKNNIEVVFSLFCERFKITPT |
| | | NDISLINDQLKDDVAPSEYQKKVDFLKQYLPVIHYLDLEITNEKFSKEKD |
| | | KEKAKRKYFRENFIALIKAIGCLRDFYTHHYHKPIFFDVNLFDLLDELLIT |
| | | VVFDVRKRKMKGDQTRHLLKKGLHSEIEELVKLKTIHLKEQKKEGKRV |
| | | NLDSEAIENAVENDAFYHLLYKEGVSRNYQSKLISETENENNLNISESGL |
| | | LFLLSIFLQRKENEQLRANIKGYKGKGKDNEKPIDRNNNSLKFMATHWV |
| | | FNYLSVKTIKHRLNTTFQKETLLIQIADELSKVPDALYQTFTKEQKEEFVE |
| | | DVNEYLKDGDSSESLEKSIIIHPVIRKRYENKFNYFSLRFLDEFVNFPSLRF |
| | | QIYLGNYVQDRRVKTISGTNYTTERMIKEKINVFGKLSEISQIKSDYFGSS |
| | | NEDTHWEMFPNPSYNFVGNNIPIYINLEKSNVEGAGKLNGLLNQLHKNQ |
| | | EIKNRKENKPSKTEMISELHEGIIFGKPTAVLSMNEMQALLHEVLVNNKT |
| | | GEEVEKILINKLIERYDTIINFDPNEPLPTSKITKKLRKSTNEIKIDTDKLLR |
| | | AIESEIRVTNEKLCLIKNNVQELNDRQQKRKFVFINKELGQEATWLADDI |
| | | KRFMPKETKENWKGYQHSLLQELLAFYESKPKEAHNFLNTHWNIKDNN |
| | | YPFNEWLVKAFQNNKFDRFYRVYLSNRVKYFESLKTQISGFKNNRKLLN |
| | | KFLKQQFVWSIFYKRLYEISTTENQKEQLLLKPLVFPRGIFDEKPTYIKGE |
| | | NIVESPNLFADWYRFIQDKNQELQKFYLWERDYKELFEQHKIALEFSTN |
| | | KYGLSEIDKFNLFKRKWDNKIKATIGQDLFLNLMNRKIIKDLYNHDVDL |
| | | SLSDYYLTQKERIEKQNKALKQSQREKQDKSENIVKDDFIWSMTVSFKT |
| | | EFIDEPSIKLKDVGKFTRFLNEEKVKRIFEYNTSKKWTKLEIEKELENYEK |
| | | IRREEIFKLFQELEEKILKENKFDGVNHPNFFMQNGNPNFKKYITQGFLIK |
| | | DKSLKKEDVEWIEGLKDKGSKNTFESNDTIKELQQKPEKIKHAFMLIYLR |
| | | NKFAHNQLPIKTYFDMICPNFDTTKSITEHIFNYLK |
| 2725811 | 9 | MAKKKHLSAADRAAQKAASSAQRSSQSKEEERIRKEAEAAAKAEAEAA |
| | | ERAKKEAQLLSTFKKDRAGEPQKRKSTAKAAGLKSTFVIEDKLLMTSFG |
| | | RGNEAVPEKRISDDVITDVSSAPAYSVTPVEKRFDICGRANNSGTTDNPV |
| | | FSKKKSGDDLIRGREALETRFFGHTFSDNIHIQLIYNVLDIEKILAVHINNI |
| | | VYALDNVRRADYSEDDDFIGYLSTQNSYAVFQNPKGINKTPGAINSINK |
| | | SREAFEQFIRNPRLGYFGSAFYNVDNNNDDNNKKANRGKAELKSEEDIY |
| | | YFLCILGAARQMLIHSNDYTKAFLYNIKDKEKADVKGFLDRTYEERINE |
| | | VNKSFIENSKKDLTIIFNAFNVKTLEEKASFAHDYYDFIVRKAQKNIGFSI |
| | | KKLREIMLEMIENADLKSSDYDTTRSKMYKHFDFSIFEYYRKNPKKAEAI |
| | | VEKLRTAADDEQKERVYVREAMALWDAIGVSFRKKVVPNMTGTAIKDI |
| | | TADPDINDSLFEGVLIGADTDYFCKAIYAFTLLIDGKEINDLLTTLINKFEN |
| | | IASFISVMEHEGIEFRFEKSYKMFEKSAEIASDLRVINSFARMQKPDPSAR |
| | | KQMYIEAAEILGTGDSEEELSEYFDNILDKNKGKKRADGKKDTSLRNFM |
| | | ASNVIESSRFRYLIRYCNAKKVREIANNRPVVRFVLDGIPDTQIDRYYASC |
| | | KGTGNPDAGKKREFLADMIASMDFRSFENVKQDDRKSSDEEKQEKEKF |
| | | KTIISLYLTVLYLLIKNLVYVNSRYCIAFHCLERDRQLHGIGKDESLTCLT |
| | | QLFLDNGWLNEHATEYLKTNIENSDDRVITYYRNSVAHLGAIRNLNEFIG |
| | | PAKQFGSYFELYHFVMQSALKKQCGLEGYTPGEKTQTYFVLVEKYGSC |
| | | CKDFIKALNVPFGYNLPRYKNLSIDGLFDMNDAREKEKGKAKDINIKNT |
| | | D |
| 2724407 | 10 | MAKDKKQDRVIGLGALAGAFGMETAEPKKEAKTEKKEKQPVKPVVAE |
| | | LISLEGMEEEKKAVAAEQPKAEIGKKSSSKAAGVKSVFTMGDGSLLMTS |
| | | FGRGNSAVLEKNVTEDNEINDINTEAPVYSAKVKENGFTVKGRASHAGS |
| | | VDDPRKSEKRPDQIDCREKIEQRFFGKTYEDNIHIQMAYNIMDIEKILAV |
| | | HVNNVVYALNNILGDEEAFYADWIGSMGWDKTYEKAMSAKESNSRFV |
| | | ADCFKNLMRSKRRAYFGDVLFDAGISKITKKEIKEARIEEERKKAYYICL |
| | | LLGKARQMTAHDLADTRSALYCLDETFDAKCKNEAKEVRVEVRAVLD |
| | | ELYYARVSKLNSDFIDTSKKDLVLLFRAFGITEPAQKKEYAGLYYDFVV |
| | | RKNYKFMGFSIKELREVITKTIPEAAVVKDEKYDTVRQKLNRLFDFAIYD |
| | | YYRKNPAEADSIVCTLRAVQSETDKQLVYRRSAKQLWKSIRATVLNHIL |
| | | PNMNGDAIKAIVADRDITADALKDVLVSPNADYFVKFLYMLTRFQNGK |
| | | EINDLITTCINKFENIASFIAVLEEKHIGRRFAQEYSVFERSAKIASQLREM |
| | | NNFARMSMKSDETATKKAMFEDAALILGYENAAELDSMLDRMLDPKG |
| | | TEKGFRNFIVNNVIESVRFDYLVRYCNPTKVRKLASNAVVLGFVLRGIPD |
| | | DQILRYYNACTGSEAKECTPRMRDELTKKLEGISFNEFRDVHQKDATAT |
| | | PAQAQDKVRKQAIIRLYLAVLYLLVKNMVYVNSRYFMAFHCLERDNSL |
| | | YTGSEINKNDYAAFARAFMQEERHRKNARAARYLDVDFANSDPQAVHE |
| | | YRNCVEHLSAIRRMDEYIADVGKMDSYFDLYHYIVQRHLSDQHDRAIRE |
| | | NKYGKIENPKMLGFIANMKRYNGCSKDMIKALNVPFAYNLARFKNLSV |
| | | DGLFDMNDTREKPKGKNCGMETEEAE |
| 2712332 | 11 | MKKKENTKQKREQLAAAKRAGFQKQEEDKRKAEALKAEQQKAAEKA |
| | | KARKALEAQIRAEDPQISDKRHSTAKAAGLKSAFTINANEVLLTAFGKG |
| | | NAAIPEKWVKDEAISSISADPMFSAELNEAQFVIRGRVNNEGNADDPRK |
| | | NASAGDDQIGIRRPLERMVFGGEFEDNIHVQIAYSILDIEKIMAVHINNIV |
| | | YAIDNLQRNEGKDFDDFIGYMGLRKSFEQFMKKDSGTDKELQDIFLRLV |
| | | KSPQMGYFGDVIYDAGIQKKAVAKIRKKQDIFDDPAVAAHYKKCYYVF |
| | | ALLGMMRQFSSHGTPENRAALYTLDAAYDRSGSDYKEKNIARSEARDA |
| | | LSGMYARRVDELNKDFLDKAKRDFVILFKLFDARTVQEKQHLVRDYYD |
| | | FIITKSYKNLGFSVKHLRELLETQTEAARFKEKEFDSVRRKLNHMFDFVIF |
| | | EHYQQHPKAAQALVECLRASTSDIEKEICYREEAVSLWASISEQIMNVLL |
| | | PKMNGNIIAHIDADGDVQEAMLNDVRISSEATCFTKLIYLLTCFLDGKEI |
| | | NDLLTTLINRFENIASFVSVLRSQKMSCDFAEKYTLFADSDVISRELRLVN |
| | | SFARMTQENANTKRIMFIEAAQILGYPGSREEMETYIDKLLEKKEKGQN |
| | | GFRNFIANNVIDSTRFKYLVRYCTPAKVRKMADNRAVVDFVLKDIPDAQ |
| | | IVRYYNGCFAADEVYREGMRDALAKLINGMSFTEMEHVRQGNNLSEEE |
| | | RIFKERKQAIIRLYLTVLYLLVKNLVYVNSRYFLAFHCVERDAMITDAKK |
| | | YEKELKADNKNKDWAMFAREHVEGRRDGRVKRYLMTNFANSDLWTL |
| | | RAFRNSTEHLNTVRNAEIYIEELRSFNSYFSIYHYLMQRQLIDQYDWERD |
| | | KGYISDDRMTGKTLRYFGLVRRHGSYCMDFVKAMNVPFAYNLPRYKN |
| | | LSIDNLFDRNKPGADDGIGPKAE |
| 2708655 | 12 | MMGNKKSVAKANGLKSTFVLGENTAYMTSFGRGNAAQPEKHIRDATV |
| | | TDIQYTFRAKTDGGRTVHIEGRVGASDVLLPDAANQLHAKDAVEQMYF |
| | | GKAFSDNIHIQIAYNIMDIKKIFGVYANIIVHTVNNLCCDGDKQDDFLGM |
| | | FKTQNRYQVAAWAHKIVSLHLVKNELRGGGFFMDQEVWRAHVRTDFK |
| | | SLNLAVNAFMKKYPQKYPYWSVKIVSDFIVQEMGIKNKVILEKAAESYV |
| | | EFETVAKRLEKSAYYFSDIFAGKDGKFDEQKAFDLLRVLGMMRQEAFH |
| | | EKNSSASWLYNLDAEADEDIKAALRTVVDTKVTGINTNFAKQNKVNLL |
| | | VLQEIYPQKSKADLVREYYDFSVRKAFKNLGFSVKTLRETMCAFDAASV |
| | | ITDKQYDTVRGKLYSLFDFVIYNYCLENEAVCNAFVEELRANLDPENKT |
| | | ALYQTLAEKVWAEIGDIVLHRILPQMHAKKIQEKSKETDAETVEMQGY |
| | | VQAPQDLSLFSKAVYCISMFLDGKEINSFLSALINKFENISSLCAVLAYNG |
| | | LETEFVAPFTFFADSQAIAEDLRYIKSIARMNKGKKATKDSPVTVKEMQ |
| | | YFDAAAVLGETDTEKVKAAFHLGDKSASTADKAFRNFVVNNVINSNRF |
| | | VYVVRFINPKNAREIMQNRALIAFVLKDIPQSQLVRYCGTAGIACNADEP |
| | | NTEAMVNALADMLLQVRFDAFSNVQQKVKADSAEAVQKEKYKAIIGL |
| | | YLTVLYLLVKTLVKINMNYAIAFGILERDCQIMNQKHGKNPKRDRDAFY |
| | | MREQQNKQYVYNARAITELFIENGWLNKRVQKSVENNAALYSDEAFYK |
| | | YRNLVAHLNVISALPKYAKDITKVKSLFDVYHYILFLSLCEDKNSNLPEA |
| | | VMKSLCKNGKTMLENAREHQTVCKDFLYGLNTPFAYNAARYINLSNRE |
| | | KFLAGFGK |
| 2725856 | 13 | MKKRVNAKEKRELRKQQQKERFRLENEAKTPAAVTEDKKEGNGKAEE |
| | | KAMIPGISPKKSSAKAAGVKSAFILSENRILMTSFGKGNQAVLEKKIEGE |
| | | RVDDLNDRPVYSAFPDGTVIGIKGWKDGMVDNPLHVNDENITRAPGSDI |
| | | LGLKDKLEARYFPNDAPFDDNIHIQLIYNILDIEKILAPFVNNTIYVINNLT |
| | | RSEGREMDLIGNISEKNSYDTFMAPETEIFDDTILEKVINQKAYFEKIITMP |
| | | QRLYLGEALYNTRLKKGQKEDESDRKRYYYTLYLLGRMRQSLFHGDRK |
| | | KQSMLYTIDEKFDSNSETEDLLKARKEARETLDGIYSVRIKKLNEEFLNL |
| | | AKKDLAVLFRMYGAKTPEQKEILVREYYDFTIHKEYKTLGFSLKVLREK |
| | | VFASPEGASVKNSCHQSMWHKFVKNFDYIAYHHYKEKPEEAASIVEAL |
| | | RSSMADAEKDGIYSAAAGRLWESIRPVVTESLLPVMDPERFKHLTEDGD |
| | | VNKTMLNGVSIGTDVHYFSKLMYMLTMFLNGKEINELLTKLINKFESIDS |
| | | FNSILSSQGLSLRYEKWYSMFLESGEIAKELRTINSFARMQKEDPDAKKT |
| | | MYVEAAQIFGFNDTDEALSDYIEYILTKDPMKKAELQSSIRKSSFLLPNE |
| | | GLRNFIANNVVESTKFKYLVRYTNPAKVSVIAHNDQILKFVLGNIPDEQI |
| | | ERYYRSCIEDASASPEAMRDSLRHLIKGVSFTDFADVNQKSNAEQNEDK |
| | | RKKQSIIGLYLAVIYLAVKNLVNINSRYTLAFHCFERDYALIKKHGYDKN |
| | | NESYKCLTEEYIELGKLNKKACRYLKQNLSNAYNQTIRFYRNEIDHLTVL |
| | | RDFPEYVRDVKKVDSYFALYHYLVQRSLKSEYEHRKLCNNLMDGNEPV |
| | | TESYFADVAKYGKYRKDFVKALNVPFGYNLPRYKNLSIEGLFDMNYPG |
| | | DKGCTGPDDSCT |
| 2712490 | 14 | MAQKQQKPKNDKRMTGLSGLNALGAAFGLEAEKPTEKAPAVREWKAE |
| | | PIKAVQEKVTADGALRNRRIKEQKEALAREGVKKHPAKAAGVKSIFTLK |
| | | EGHLLMTSFGRGNAAVLEKDVNDTVIEDIAKEPAYLAQHAPKRFDIKGR |
| | | IGIKAKVGNPVCGGRPAGDDLIGLRSRIEQRFFGKNFEDNIHIQLAYNILDI |
| | | EKILAVHINDIIYAINNMAREEDSEHGDVIGLLTMNKEWANFIKNGGEKA |
| | | EAVQNLLNKEQLSYFGTVLYDPALVKKLKTAKNDRFREIAKAALDEKK |
| | | KNAYNLLTMLGAARQSLAHDNPVIYKLDKEYDDQIKNRNDERIKPAQR |
| | | LAARDTMDALFAEAINGLNSNFLNNSKVDFHILFTVFDAKDAADKTELV |
| | | RRYYDFVVRKEYKNQGFSFTRLRENMVLSAGAKPLMNTDYDSVRGRL |
| | | YRHFDFILFNYYLDNADAASAMVEKLRACMTGEEKEALYEAEAKRVW |
| | | PLIGAKVMNRVLPLLDGTSIKRVSAGSADSDIPLDALAGTAISPADTSEFS |
| | | KLIYLLTCFEDGKEINILLTTLINKFENIDSFRQVLRELKLPSGFADAYRMF |
| | | GKSGEIARELRMINSFARMDMKNQPDAKRVLFVEAAKVLGYPDTEEKL |
| | | YKYIDGMLTATGEFEAKGKDFRNFIASNVIESDRFKYLARYANVNRVRA |
| | | IAANKNVVAFVLKGIPDTQIVRYYNSCTGADKKEAEPGMREKLTAIITGL |
| | | SFEQFAGVRTKTNNIDKMQKQAIISLYLTVLYLAVKNLVNVNSRYFLAV |
| | | HCAERDANLFDRERFHASFEAKGDMREFAKERAGLRRIEEHNAEIEAWA |
| | | KAKAEREGKPAERKPRERRKLYTEDGRRINVRARSYLEQDYYNGDAFII |
| | | REFRNCAEHLGAVRNAADYIKDIGSFDSWFEMYHYIVQRYLEGQYNFY |
| | | KGQNKVSDEEMTGKTLEYFERVNATQSYCMDFVKALCVPFAYNLPRFK |
| | | NLTIKDLFDMHD |
| 2712135 | 15 | MPRLKAKEKRELQKKNMANRYAKENISVIGTVTKADEVPDEPELDETK |
| | | EIIKTKASTAKKAGVKSTFILDGNKLLMTSFGRGNEAILEKRIDNEEITDL |
| | | NNKPQFELGKGSLVNEYSVVGRNRKEGTAVNPNHKNTENDRYQKMPG |
| | | EDNIGIKGYLEKTIFDNCEDCKDNIHVQIAYNILDIEKILSPCVNNVVHCIA |
| | | NVTRKNKMSEALDLGMDIHEDNPYDALVKPEKYFDEDKTARVTSSYEK |
| | | FISLISSPRRRYFGEALYDPSEHNEKKWIDTEDDLKRYYYELYILSMLRQS |
| | | LFHGEVSDSADIYKIDERFDGDYQAKPTRVTARKEAREQLDIIYKKRVD |
| | | KLNNNFIKMASEKDLPLLFEIYHADKKEEKEKIVKEYYNFVVRKEYKNS |
| | | GLSIKKLREKILYASEGVLSKDGETIRSAVEHSAWSKFVKLFNFVIDKYY |
| | | NEHQEELRNVVEELRGDSTDEEKDNTYRIEADRLWQIIREHIMNTVVPN |
| | | LNESAIKEIKAEKGIDKNILSNIGISGDSNYFCKMIFVLCMFLDGKEINDLL |
| | | TQLINKEDNISSENDVMDAYNIKYKYSKDYSIFNDSKRIRIELGVVKNLA |
| | | CMLENDKDKDKKKKGDKTIPEKNGIERVMYLEAARALGVSCDNEELNE |
| | | YISLIITTDEEDIKAIKERLKYDFKRFPMPKKGFRNFVINNVIKSKRFRYLA |
| | | RYINPKDFELIAKNEKIVKFVLKEIPEKQIARYYQSCLEPGISIDNIKPSDYE |
| | | MMRNQLAKTISRLKFTDFTSVDQSSNALENEDKSKALNLIGLYLTIPYLF |
| | | VKNLIYVNARYSMAFHSFERDHKIITGKKFNKHKLTYDVITKKFIEEKRI |
| | | NSRASKYIQTNMEKSNAFCIHKYRVCVAHLLVLDNITKYINDIDTIDSYFS |
| | | AYHYIAQRFICDQYYSEEGTEYRDANSDAFFESIKKYHCYRKDFVKALNI |
| | | PFGYNLPRYKNLSICELFDRNNMGKQIPLQQCAD |
| 2108577 | 68 | NPSRVKIPLCDKCSSASGQNNTKLCDSCQKRQSAAKDLAVEIKQIVNQII |
| | | CEELANQNKDFGKKVINSKANSKDGSKKSKEYNIKMEDLDLLIFCLKKK |
| | | FRSNVKFQDETNVDHKFCFLTDLGDYLRSELCLSPELSQSSTPSTSSPSSK |
| | | HSSFLDAYRQWVRSYVRKKKKYTKRSIENNKLVVDYSDEASLSPRKKC |
| | | LLKWGDEYRKNGKIDLRTEEKLLGMDELIESINKVRKDHGEPIIDDARG |
| | | NEQMNYEFNVAVKRAIQSRKKAVFGLVKESSLDNGSNLINKINSTNEIN |
| | | SINERNLENRKNANLTTYFNEVVKYFEHYFPIKSKKYDIIKNKKGKRKGE |
| | | ELKKSLRYLEADTIKSTVLSQIQNAVRSQFLQQGKSELYDLKKETTCSDQ |
| | | LSLLKRDEFFVLNLIEACAFATNNIRNIVGQTQCEDNQRKDNQRKDIQRE |
| | | DILGNDITKGVIDCILKNEKDPSPNKSVINSASNSIFNKELFNSFYGRELSV |
| | | KEEDSVSQFVKTIWAMRGSVQQIRNEIVHFHMGSIDKIFNLENFEYFDQK |
| | | SDSNQTESSKLIVSSVSSEPTQSTQSAQSTEKSNEPNRSNEPYTETVFKDC |
| | | LENDLNRVSELFIEKLQSNDVLNYYRAHDIQHLLEKLEFSLCKCSIPFAPG |
| | | FKNIYEAGKYLQNAGNQHFINNQQSINNQRDERTKRSQSNECNQSNQTD |
| | | VENNPFELRHYLLKDKDAAKTAQAKNYSAYRFLLKILYDYCFLYQFLN |
| | | DKQAFEEAVRFTLDNNKAANSRKANKNEFAFNGIPYFDPSQSIEAYFSEI |
| | | HSLAVHEENKKKEKDGEQRLNTQRFITKVFVKGFDNYLKEIKVGFLLNS |
| | | NQDQPKKLTQEDYSPLAEFVKVKSCIDKKQNSHIAFYTFCKLLDAQHLS |
| | | DLRNEFIKYKASNRTTNTETSKPNDKRKFDYIIDILGLCLLSVDSLKCNDL |
| | | NQRMDECTKQSWKSDLESFVDADLLSFIDRGQPDSEVINLYKQSDDYTP |
| | | VAHASIRLLRKYGTKELLKQIIGTDYIVSVGDYNEWQKFKDIAVNKECT |
| | | VIEDYINTRTRLHDEWKSLVQEKKNQAATRGKGKYKPNKDKSNKDNEA |
| | | IISKSTIDNFILKNKAEYVDVCNKIERYNWLDNKIHLVHLRQLHNLMIQIL |
| | | SRLTRFVALWDRDFVKFDRTFTDSQYIFTGVVSLASICNGKESNLEDKVK |
| | | SRSDNEPYKSKDILSLQTDTKAKREVYNNLFFKQNKDAFSDRNYIAHYN |
| | | YITKGGTKSILDLIKRVKKLMGYDRKLHNAVVKSIINVFDQNGMELRMS |
| | | IDSNHELTLEENAIIPKTINHLERTDISTNLVSEEYCKLCQKMLEMKK |
| 2132026 | 69 | MRVSKVKVTVNNKDTMVLMHRSAEKGALIYDTIDKKDRTEEIIPQKKQ |
| | | KSFELSIQNKTLLRYEAIKKQKLDRSKYKVVEDIFKYRKLPAKITLEDILP |
| | | FLNHKFHLPVKYQKEGQWVQFELSTCILQAVEHQNLDELHPYYDWSKW |
| | | YINDKSGKLKKSITNNHIQIGDGESKRQKALLMWETDFTDKGELDLETY |
| | | HEKFIIDTLAASLRSVEKGELRDNGTPKETNSYHRNLKKVLQNHQALIFG |
| | | RRETPNQENRDNKQLAIYNLEVVKYLEHYFPIKSNVRKNSAADIVYYLK |
| | | SETIKSTVTHQLENAMRANLLRKGKYQHHDLKADATSENLTNLKRDEA |
| | | FVMNLITTSAFAANNIRNIIDPEQIKDIVGKDDFIRSLKKDAFNKHIYGLFF |
| | | PVGAEMIEPVSLWAMRGAVQQIRNNVAHYKKDALATIFNITKFEYPGAE |
| | | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSGRYFLLKIIYNYLFLPEFINNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRAASES |
| | | ELFIHLTEIIELCLLHADNVPADYWQIYKNKEQCLQRLQPFIAENSDYTN |
| | | WGDLYVQTDDETPVVHSGIELSVKYGTASLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHDAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKIIFHLGTKKESDNIYTFQVPT |
| | | AYCNMCKQLLEMKN |
| 3016461 | 70 | LKKKVKARDLREAKKQEKIAVYSAAAKAAVGDKSAVEAKPVRPVEAA |
| | | HEDKAVGNKKSVNKAAGLKSTLVYGDKLYMTSFGRGNSAVVEQRIDTS |
| | | DYSVSDMNASPSLRVSEAGKINVSFSSLRQFVNAGQLSAVNPLYGGKDK |
| | | PQKKTGQDMLGLKDKLEERYFGMTFNDNIHIQIIHNILDIEKILAVYTTNI |
| | | AVALDHMVDEEIDAEREEFIGYMNTLNTYEVFMNPEKKFGAGDKRVKN |
| | | ITESRDKFEKLLATKRMGYFGFDYDSNGKDKHKNEELKKRLYHLVAFA |
| | | GQLRQWSFHGDTGINPNSWLYRLDDKKLDQEYRDTLDYYFDIRFDEINE |
| | | KFVEQNATNLCILRELFPNDDFKAVADLYYDFIVIKAQKNIGFSLKKLRE |
| | | KMLESEEATPVKAQDMDSVRSKLYKLMDFCIFKYYTEHPEISNKNVDIL |
| | | RASVSDEQKESFYTDEAYRLWGIFGGQFLSFCSRINKWVTGEHQKEVLR |
| | | YIDKDAYRRQSSVSYFSKLLYVMCFFLDGKEINDLLTTLINKEDNIASFIN |
| | | TAKELGINVSFVKDYEFFNSSDRYVPELNIVKNISRMKKPAESVKKEMY |
| | | RDALAILGVPSNMSREAFDAELEKILEKKIDPKTGKPIKGGNPFRNFIANN |
| | | VIDNRRFIYVIKFCNPENVRRLVNNINVTEFVLRTIPEAQIDRYYKSCIVSE |
| | | LNPVPDKKISELAKMRKDINFGEFRGVQQKAHTREEALEKERFKAVIGL |
| | | YLTVVYLLVKNLVNVNARYVMAFHCLERDALLYGENVGNDYLNLSET |
| | | LCSENENSRSGYLARNRRLRECVKQDISNAHTLKVRNYRNNVAHLSAV |
| | | RNCAEYIGDITKIDSYFALYHYIIQRLLAKGTDRTAADFESRYPYFAQLFK |
| | | WNTYVKDMVKALNAPFGYNLPRFKNLSIDDLFDRNEQARKKSEMLDK |
| 3024180 | 71 | MKKSNSKKMGIKIKTNNKNDIYFFDNKDFKKDNQNYLMLSDEENKLIK |
| | | KSNTAQFDFLDTHINKRIDIIKGKNIAEYENKYSINKENLLNQCLIKKYC |
| | | YKSDVSSNLVIQLIHNNEDIKKIVSLYVNDVIYSLSSINKDNYDVISKISPS |
| | | FNMKNNKIYTNSGIYNNSDLYNILNNLKYRQTLYGLENKNKAILFEYLK |
| | | AISSLRNLVAHFNKTDVYNLNLLKNELSNYILIKGKEFLKNSALNIDLITK |
| | | LYSKENINEEVKQYYLYVLNQKDKNIGLSIKKIMEIARNIYPSNIENEEKN |
| | | KKYNVLLKYHLFNYLTNNKELTNEYIDKLRKSDYEDKDNIYYELFKKIG |
| | | KKEFDRVENIIDKIDFKEEVSINLKDIEKEILNVKYNWNYFILQIYALSKFL |
| | | TIKETNVLTSSLINKFQSMLDLYYVSKSINFNIKDCIKTNYLICSVDEMED |
| | | VINQLRIINSSRRYINIKKSKCDERFVIDGLKIFKNNYNNEELKKLLNEKIN |
| | | NKIYKSKKIKNPLKNFIRNNVIKTKEFNYLIRYSDPGKLSNILYKNVNLVK |
| | | FLFEQFTDSEKEKYKLNNVNEKTISEITLNEIIKNLKSGNMDYSINVKAYV |
| | | KCIYFLIKNMVNINSYYFIAFSFYARDMMLINSDYKEYSLKLFNYVKSKD |
| | | RDETRMTIKNFNSIKDKNIFDRYRNIVEHININKLLYDTKFPDSFKYSNYF |
| | | KIYHYCIQKELLNIEYNNKFLNECKSNINEYNKYSKNLLYVLNSIFIYNYA |
| | | RFKDLSVEEIFMRKYN |
| 3028750 | 72 | MSKEKARDKRLRQQEEKKQKIQSVNEKREAERLRKEQEEARAEKAKEE |
| | | AREKAQFENEIAQIEREAPNSAKRKKSEAKAAGLKSTFVLAADKLMMSS |
| | | FGNGNSAIREKYIEDGHISNISEIPVLDVKNDEDEKKFLISGRLVSNATAD |
| | | NPLKTKRPTGMDQIKCKDKLEKLYYGKCFDDNIHIQLIYNILDIEKILTVH |
| | | VNNVVFELNNILRNEGNEFSDLIGEGLGREKTYADFVKSGAKKFDTFKK |
| | | FAYCKQLGYFGGTLLIPRERDRKKVEDVDNVHLKRTYDLLSALSVLRQA |
| | | TAHGEASTRAQIFNFDSEKSKKSKYYLEIRTALDNLYEDRVHTLNERFLE |
| | | RAAKDLKIIFDIFSVESASEKQRVIEEYYDFVVLKSYKNTGFSIKHLRELII |
| | | QNYAGNIADHEYDSVRPRLNRMLDFIIFEYYAKTENQESASYLVDSLRA |
| | | ADRDSVKEALYRRAAGHIWKAIGNAVSDRLLPKMNGDYIGALSEVLVD |
| | | DMSISSVAIPETAHVFTEMVYLLTIFLDGKEINDLLTQLVSKFEIIESLNAV |
| | | LREEGLETGFCSDYTMFEKSGEISRQLRAVNSFARMSEEDPSVKMVLFV |
| | | EAALILGYSGNREELECYVEELFNKDGGKRLTDGRKDNGFRNFIINNVIT |
| | | SDRFKYLVRYGNPEKIRKIANNRKVVEFVLKAIPDAQIRFYYNSCNGTNA |
| | | AYESAMRDDLADKITGISFLDFENVHQNDKGYNAWHGDKERKKNIIRL |
| | | YLTVLYLLMKNLVYVNSRYYLAFHCAERDAMIWDDKKYKFTNESKCA |
| | | DRVNFAKAWIEENSRDTRAKHYLIDNFKNADIWAISTFRNCAEHLNAVR |
| | | NMDSYINDIAHFDSYFELYHYLVQRQLMKEFDYETTTPAKDNNNRMLL |
| | | EADELTGKTEKYLDIVRKYNFYCKDFVKALNVPFAYNLARYKNLSIDAL |
| | | FDRNNYLGATKESIKSEE |
| 3028811 | 73 | MVKKKLTAKERREQQKNDQQAVRAEREAQKLARAAAAEEAGRRAAA |
| | | EAEAERLRKEEEARLAHVAELRAQAKANKERRKSTAKAAGLKSAIYVN |
| | | GDELLLTAFGRGNEAFVSKRVKGSEITEAAGGRASYTAEMGDKQFLLSG |
| | | RTQGQRDDPRSSAKKSGDDMIGARAALEKRYFGHACEDNIHIQIAYCIQ |
| | | DIEKLLAIHTNNIVYELNNLRRNPDAEYDDFIGYLSVRDEFAVFMDDNRR |
| | | FADKDKDVKKNVENSRQNFRDFIKNPRLGYFGSAFVDVGTDTRARMKS |
| | | EQKPDADIYATLCLLGTLRQCMAHSYNSSRSDIYRIDKKDGAAWDEARS |
| | | LLDRTYNARVEALNRDFLSRAGKNLTILFGALGADTLEKKTAFVRDYYD |
| | | FEVRKSAKNMGFSLKGLRERMLLLPDAAVNGEPVRTHKKYDSVRSKLN |
| | | KSLDFAVYEYYKGNPARAEDMVESLRAAQCEAEKEELYAAEAKRLWK |
| | | EMKPVLFDHILPAMDGDRIGKMKPDPEITADMLQGACIGTDASYFSKLIY |
| | | LVTLFQDGKEINDLLTQLLSKFENIAAFLTVSPLCTAGDFTNDYSFFAKSG |
| | | EVAGELRVINSFARMELPEAAANVEMYVEAAVILGAPGSRDELKEYFKN |
| | | LLDKNGGPLKPNGKKDNGLRNFIASNVVSSRRFKYLVRYINPEKARALA |
| | | RNRTVMDFVLNEIPDEQIARYWNGTRSANAAYDESMRADLAGLITGMS |
| | | FSEFEDAAEYQAGRTDEQRSRKEQQQTVIRLYLAVLYLLVKNLVYVNSR |
| | | YFLAFHVLERDAQLLGSKAGYNGTPYAALTRWFVKENRLNRHATAYLT |
| | | VNLEHSDDKCISWFRNKAAHLDAIRNADKYIADVREVTSYFGLYHYLM |
| | | QRALEDKYIHSGKEIPLYTRTYLNKVRTYKTYCKDMVKALNVPFGYNL |
| | | ARYKNLSIEGLFDRNRPGDGKEDPQVSE |
| 3035346 | 74 | MSKKNNPRELREARKAKMKEKYASKAVEKKEVIEEKAAAEPKSDKNK |
| | | KSLSKAAGLKSVFYAGENLYMTSFGRGNTAVVEQKINTSDYSFSSMNKS |
| | | PNLRVNNADEINVSFSSTRPFVKENKLTADNPLHEGKSSKPKATGKDILG |
| | | LKDTLEKRYFGKVFDDNIHIQIIYNILDIEKILAEYITNATIAIDYLCDTEAN |
| | | GVYGDFIGYMNTFNTYDVFYNPSKNPRLTDRDIKNINRSRTNFEKLIKSN |
| | | RLGYFGFEKEDKKRLYHLMALAGQLRQWCFHSYGNAARTWLYNLDNR |
| | | NLAAEYYDTLDYFFDERFKEINNDFISKNSVNLFILKDIFPEEELTEVIKLY |
| | | YDFIVVKSYKNMGFSITKLREKMLEQTGAVAITSKDMDSVRSKLYKLID |
| | | FCIFYGYYKDEKKSAENVDYLRSCLNEDAKENFYAIEAKKLWEKYGNL |
| | | FLDFCNIVSETNIKNLSNDVTKKKHTEILSRYIDFEEYRNSSNVSYFSKLM |
| | | YVMCFFLDGKEINELLTTLINKFDNISSFIATAKELDIDVNFVADYSFEND |
| | | CGKYVNEINIVKNISRMKKPSAKAKKVMFRDALTILGMPHNMTEEQFEA |
| | | EIEKLVEVEYDVTGKKIKKPHDYRNFICNNIIENRRFVYIIKFCNPGSVRKI |
| | | ADNTAVTKFVLKRIDEKQIERYYISCMEAPDVNASLDYKINQLAEMMKR |
| | | MDFGQFSNVRQISSNPSEKQRKERYKAIVGLYLTVVYHLVKNLVNVNA |
| | | RYVMAFHSLERDAALYNYKLEKNYTGLAKLLCDEEENSRSGYLARNKR |
| | | LRECVKQDIENAKELDVQNYRNNVAHLTAIRRVGDFVGDTTKIDSYFAL |
| | | YHYLMQRLLLEKSNADHKYNKQLNQWHTYVKDYVKALNSPFGYNIPR |
| | | FKALTIEQLFDRNEQKRTLSE |
| 3037546 | 75 | MSKKKSPRELREARKAKMKEKYASKAVEKKEVIEEKAAAEPKSDKNKK |
| | | SLSKAAGLKSVFYAGENLYMTSFGRGNTAVVEQKINTSDYSFSSMNKSP |
| | | NLRVNNADEINVSFSSTRPFVKENKLTADNPLHEGKSSKPKATGKDILGL |
| | | KDTLEKRYFGKVFDDNIHIQIIYNILDIEKILAEYITNATIAIDYLCDTEAN |
| | | GVYGDFIGYMNTFNTYDVFYNPSKNPRLTEKDIKNINRSRTNFEKLIKSN |
| | | RLGYFGFEKEDKKRLYHLMALAGQLRQWCFHDANYKSNDINKEEYVK |
| | | GKIRTWLYNLDNRNLAAEYYDTLDYFFDKRFKEINNDFVSKNSVNLFIL |
| | | KDIFPEEELTEVIKLYYDFIVVKSYKNMGFSITKLREKMLEQTGATAITST |
| | | GMDSVRSKLYKLIDFCIFYGYYKDEKKSAENVDYLRSCLNEDAKENFYA |
| | | IEAKKLWEKYGKLFLDFCNIVSGSNIKRLQNCDIKELDRYIDFEEYRNSS |
| | | NVSYFSKLMYVVCFFLDGKEINELLTTLINKEDNISSFIATAKELDIDVNF |
| | | VADYSFFNDCGKYVNEINIVKNISRMKKPSAKAKKVMFRDALTILGMPY |
| | | NMTEEQFEAEIEKLVEVEYDVTGKKIKKPHDYRNFICNNIIENRRFVYIIK |
| | | FCNPGSVRKIADNTAVTKFVLKRIDEKQIERYYISCMEAPDVNASFEYKI |
| | | NQLAEMMKRMDFGQFSNVRQKTSNPLENQRKERYKAIVGLYLTVVYH |
| | | LVKNLVNVNARYVMAFHSLERDAALYNYKLEKNYTGLAKLLCDEEEN |
| | | SRSGYLARNKRLRECVKQDIENAKELDVQNYRNNVAHLTAIRRVGDFV |
| | | GDTTKIDSYFALYHYLMQRLLLEKSNADHKYNKQLIQWHTYVKDYVK |
| | | ALNSPFGYNIPRFKALTIESLFDRNE |
| 3040041 | 76 | MKKKVKARDIRETKQNKKMNEFADRDKALREAEAAKKAAEEKLRAEK |
| | | ELQEKISRIRVLQSNTKASPFTRRKSNVKANGLKSVLFMDDEVLMTSFGR |
| | | GNKAIEEIRVQDGKMTRLNDTPVFDAIDQTSHYDVKRTVRIDADPGVISK |
| | | HEVSEKRAQQLPHADLIGAKDILETNFFGAPQAGNIHIQVIHNILDIEKQL |
| | | SGFVNNAIYSINNVIGKVTEEGYDVDMIGSLGVTGGGATGKDWFEDFCA |
| | | NRRLLYFGPVFYEGCAGKKENKKKETLRAKEQVAAILELLGGMRQSLT |
| | | HGVGSKNGSHWLYSQDTQLQPRHKDTLDMFYESKVSELNDNFLKQSGK |
| | | DLTVAMSLMGAATMEEMTRVAEDFYRHVVRKDARNLGFSLKKLREILI |
| | | DTRMPQLRSKDYDSTRSKVNRMLDFVLFRYYVREESGIRALELFVDRLR |
| | | GLSQEGDPETLEKKRAMIYLEEAELTWPKVKERFENTVKKLGDGSRIAQ |
| | | MTVDPAISGIDLSRVRVSAANTSYFAKLLYMLSMFLDGKEINEMLSSIQN |
| | | KLENIGSFMAIIKRHGLKDLSGTRYELLMDADRLAEELRVVRSFARMER |
| | | PAPGAKESMILDAGYLIGQTVDNETLRAVFGDRYVNARGKKDSELAAE |
| | | YPQFIKRNGRFDRNRFFANDTRLRNFLIKNVVESDRFRYIIRYANAKKVR |
| | | EFAQSPALVDHVLRRMPETQIDRYFKACFPGYDNSNREDSIYALRETIRT |
| | | LKFDQFKELRIDDEEAGERKQRFQAVIGLYLTVLYVITKNLVNINARYTI |
| | | AVHCWERDHAMVLGLTNMFGRDYEGSHEDIYGMTDRYIERMEERKAE |
| | | CVRKLRAHENGAKRFNGHALNYVKANRSVWNDTEAAYHMFRLYRNAI |
| | | AHMMAVTNAYKYIPEMKKVDSYFAVYHYAMQRYIMDHGGYIANRIEE |
| | | RSETAKNYTEAVRRYHSFNKDWLHAVNAPFAYNLPRYKNLSIEALFDK |
| | | NEASAESDGKEV |
| 2143404 | 77 | MKIAKDNGRKTCVAKKWNQNQIKGMIYKSPKPGVLSMDSLHNELKKK |
| | | DGSVKFLFSIFNTIQVGQEPHKPNKDNYVDDPEKYAKKEKNYNVKMEN |
| | | YNTRKLYSKYVDMIRKEVNNVLINGKKYNDKRFKSNDEIFRSIRNANID |
| | | REIKFKTTTLVDIALRKSFKEYREEAICFIECIRKREINDNEKQFISRFLDEL |
| | | RKDFNRLDTSPLFSFLDEKVTESNNGTLIDNIKTVSRITNKILTNPKSFKSN |
| | | EKIFEGLKNASIDSNINPTELIDNALLEDYKEYKEEARCFIKCIGKKDIAEN |
| | | EKELITQFIEKLRDEYDNNAYSTKCNGTRILNSIECQNTVVQLNNNVVSL |
| | | PLADNNRLKKNEKDGLNKELLEYAVLAEDDRMEMLRKLRRLLDLYFA |
| | | VPDEYDNEASYTLPDEIDTGDFDVWDRHSEGKREDKDFVRVPEELKEA |
| | | NEENKTLEILDSKKLKYDLDNSIRRKNMNGYRFAHKIVGVTPDGVFYDE |
| | | KNINDMWIHHIENAIERVLRHRVLDNLYKLKVGYLREKIWKDMLNYISI |
| | | KYIAVGKIVFNFAMDDLGNEDGDCTLGENTSLEGITSFDLEAIKAQETM |
| | | QRDLAVNVAFAINNFANATVDLDEHNSDLLTVRIKKDNKMKEGEKILEE |
| | | LLKYNGNEEQIRKSILQFFGGYSSWIESDAINKLNDISVCKLIDGIRNAMY |
| | | SMRNRSFHFKTFDEKKYSEEIQSLIGTIFEEDAKKCVIQEKNKFYSNNLH |
| | | MFYKVSDLENTLDALYNDVHTRSSQIPSFNSVIVRKNFKAFISTELGIKNN |
| | | SFESSDDELKWQNALYYVFKEIYYNKFIHDNSIKQAFEEKLNDYGKTIKD |
| | | DSESRAYDNFKTKIRRLKKYSFAEMCQNIMTEYNLQNNQRRTVSSSRDS |
| | | LLEKDIYMHYPLLLKKIIAKTFASYIKNSNEYGYLLNPSTHISEIAIEDFLP |
| | | DWQSEKYKQLISELSKNQELQAWYITGKFLNGRSLNQLIGSIKSYIQYID |
| | | DVRKRAKIAKNKINIRFNNENEKKALEVLEVCLKMSGAVSHELTDYFKD |
| | | EEEYASYLSGYVDYTSYSDHMENHSSALVAFCNEHKCDVYADGKNPIV |
| | | QKNVIFAKMFGADSVLSKCVAKVTSDNLDTYYEKKENISNYKVKGICSD |
| | | SDEQRRLIEYQQLANKIELRNLMEYSEIINELLGQLNNWSYLRERDLLYY |
| | | QLGFHYMCLQNDFKKPESYDCIKRENDTSLKGAILNQITGMYINGIGIYN |
| | | SKGNIIENSSLGGAGKKIGCFLQYGNELLGDKKGEDLYKAGLEIFEVYKE |
| | | HANIINVRNDIEHFRFYSSDTISIMGLYSEVFDRFFTYDMKYQKNVMNMF |
| | | SNILLKHNILPYVKFGTGVKKVGKEDKEAARLIVTMLESERFKYKVDGG |
| | | IVVNADAKNKAFLNDVFSILSYNDNYIIKTDAKLRTHDVKVDKEKPNKN |
| | | NNENDNNRNHRKKKNRGEREYKDKESNSFMGGVFDNFDLESAGFKPTN |
| | | TKKKK |
| 2119983 | 78 | LKISKESHRKTGVGVSEQTGTIGLLYKNPLDKHEKIEDVVKQRANSTKRL |
| | | YNLFGTGETSKDISKNAKEVAKVVNRAIGSLKFNMKNNQRECIDKELKS |
| | | QQVISAIEKSKKYDFKENDVDEALDRLLKKSFRQAKSKNALRKLLMSTL |
| | | VDNKALSTDDIATIQNDFVKKLVADYNKSNIKNNIPKALRNQNMVVQP |
| | | DSSGILSLSQTRTDGKNNKTKDIKISEKDAFRQFLSDYAVLDDEVRHQLR |
| | | LKLRRLVDLYFYGEDTFIKDDFDEWKVHEDNKSNTEPFVTKVTVKKNT |
| | | KDNKEFEILNVDATADVIRSENISRYRQALDYVNSNKQFFFTDEMLNKF |
| | | WIHHIESEVERIYKRINAKTGDYKFQRGYLSEKVWKGIINYLSIKYIAEGK |
| | | AVYNYAMTALSLDNDSKEFGKLDERVSKGISSFEYERIKAEETLQRECA |
| | | VYVAFAANHLANATVVLDDVNTDFLLLKNKELDAKAIEKGEKSLAECA |
| | | KAKPDTLRNILQFFGGQSSWKDFDFTGCDEIQLLDDLKEMIYSLRNSSFH |
| | | FKTENIADNVKNKDLINAMFAFECVKAGTVQKNKMYSNNVSVFYSETD |
| | | IEKLLGSLYSRINERASQVPSFNTVFVRKNFPEYLKNQKLTPAFTQDDILK |
| | | WQSAVYYLYKEIYYNGFLQDKTAFDLLTKYVRSLDENCADKQQASAN |
| | | KDFKVAFELYSKSGSLSETCQMIMTEYNNQNKGNRVGISARTQKADRLI |
| | | FQHYKMILFAGLQSAFTEYIKAHSDIYGFIFKPAVKENIPEVESFLPNYRT |
| | | NQYNELVNLFKEEIELQKWYITGRLLNSKQVNQLIGSFRSYKQYVYDVV |
| | | RRAKENGNQLINAEISVDADNIIKVLDVCTKLNGVTSNCLEDYFADSDE |
| | | YAKYIGRFIDFGLKKGEELANEKLASFCQRELGDRKVGTYHDGINPILNR |
| | | NVIQCKLYGASDIISKAGILPVDYEIIKKYIGMTKDINGYQESGVCVTKDD |
| | | QQKLKKYQELKNMVELRNIVEYSEIINELQGQLINWGYLRERDLMYFQL |
| | | GFHYLCLKNSSDKPEGYDKAGEMIGTILYQIVAMYTNGLSMVDAKGKS |
| | | KRNAKASAGKKLESFFSYSAEMTDNKYGLYLAGLELFENVSEHDQCVK |
| | | LRNDIEHFHYYAVQDRSMLDMYSEVFDRFFTYDMKYAKNVPNMLYNIL |
| | | LQHLVVPNFQFGTGKKQVGDKNKQTKSRATILLAEKNGLASEKFTYKL |
| | | DGGKSTVMLSAKSNDFLSDVAAILYYPDSAP |
| 2119840 | 79 | VEGFFIDIPNELNSEKQPDAVKKKELTESFRADIRSKNIACYRYSLAVIEN |
| | | DTEGLFFSEKEINKFFVHHIENSVERILNNRLLDNLDYKLKVSYLCEKVW |
| | | KDIINYFSVKYIAFGKAVFNFGMEELLSNDAEISMGIIPDEIKKGLNSFDY |
| | | ESIKARETLQRELAVNVMFSANNLARATVDLSEKSDFLLYSYDKDYKKD |
| | | PENKKSDIDKYRLSDNDSETLRSILQFFIGKSKWDEKLFLDTYGSEYASEF |
| | | LNDLKNCIYYLRNESFHFSSAKSSFNWNKNLIGKMFEQEANACFIVEKD |
| | | KFYSNNLPMFYDIDYLRRALAKLYANDVPRASQIPAFNNVCVRKNFPKL |
| | | LEEKMKLFALANISDADLEKWQSALYYLFKEIYYNDFIQDEKSKGMFIS |
| | | AINNIKQDVEKRVEDIRKRLVIENSDLNDFQIKKKLDKYPEIAEKNAVNN |
| | | FCIRLNEIKQDNDLSQICQMFMTEYSSQNNKRTVRSSKDSILDKEIFKHY |
| | | KVLLMHGLENALVSFIKSKDEYAFILKKPVLREKPSAEDFLPNFTTNKYD |
| | | CLIKAVKNDPRLQSWYITGRFLNGKMLNQLVGSMRSYIQYVESVERRA |
| | | NNTQNVLHKDETDMKNYIKEACKVIEICILLSGTFTNNISDYFKDDREYS |
| | | DYINYIHKYVDYEDLRQGEIFFNEGGKPIVNRNVIMAKLYAPDTVLENC |
| | | VKKVNDEDIKNYMLMSGEIDSYRVKKEITSKDEQIKILDYQKIKNKVELR |
| | | TLVEYGEVINELLGQLVNWSYLRERDLFYFQLGFHYACLNNDSAKPDEY |
| | | VSLSYKEGRVINNAILYQIAAMYIYGIKLFAPKKNELQGKFDAVTGQAG |
| | | LKIPAFERFSERTTGDKDCLYNAGLELFEVINEHDNIIEIRNYIDHFKYYN |
| | | TLSDTKSIVDIYSEVFDRFFTYDMKYQKNVMNMLGNILLKHNVIPEFVV |
| | | ETGKKKVSKDGEKLRANIEVRSLEADTFTYNYLERGNQVSLIIAAKNKE |
| | | YLETIAKILYFPEKKSTVKYIIKEADSVKVNGENSRTEKTNGKKSKDNKS |
| | | KDNKRNNNIKVGDAYKSYSESESCGSSMADLLKNIKL |
| 2113610 | 80 | ESLEIIKNDKERLFFDDFELNKCFVHYIENFVEKRFENIRCSKLYMLETGN |
| | | ISEKAWKSLLSYLTVNYIAIGKAVYNFSMSDISKTEGPMNLGVVEDKWK |
| | | TGISSFEYEKIKAEETLQRDIAVYVSFAVNNLSKATIKPEKGKEDILLLGD |
| | | EELKSVLLSSLSGLTEEDALLRNIMMYFGGISIWKEFFENRFSNEYSKFDF |
| | | YKDIIHILYAVRNESFHFTTANVIEDKWNTKLISDIFNNECDSISRVQKDK |
| | | FYSNNLHKFYSEDKLKKVLNFLYDEYTIRPSQIPAFNKVFVKNDFSAYLD |
| | | ANFVTSYSWSDSEKKEWEAGLYFLFKEVYYNRFLRDITVEKFCETVKQL |
| | | DILGEKQKFAHKNFNSRLEKLKKEGFSLPEICQILTVEKNLQNKNRKVKT |
| | | SKSEKENPDIFKHYTEILESGLRQAFTSYVNEKLSFLNETKRNSNVDSGE |
| | | VFKTEFLKDWKGKKYDSLKAKISQETKYQKWYILGKFLNPKQLNLLNG |
| | | AVKHYIQYSKDVERRAKQTGNPLSADSSSHRKECEIISEILDMDMQLIGK |
| | | VFRDNYNEENNSLSPDDVILDYFDSTDDYAKYVSLFVDYGYESNKVDD |
| | | DATRPMGYLQAFCNTDTSDGQKIGIYYDDKKPIPNRNIIISKLFGLPFQND |
| | | KFKVLNRVTEHDIREYYYKKDELKEYKKTGKCNTFEDQKKLKDFQELK |
| | | NRIELYSMVDYSEIVNELLGQLVNWVYIRERDLMYFQLGFHYLCLKSNC |
| | | KFPEQYKTITIGNQGTINGAILYQLVATNTNGLPVYTLDDDGGDVDAYK |
| | | YANKSTGAKISKCFLKYSEKIGYSEENNIYDAGLELFQNMSEERDINEFR |
| | | DYIDHFKYFVDEKRSILDLYSEMFDRFMAYDQKYRKNVPNMLTNILKR |
| | | HFIITHLAFKSGEKEVGGGNKSTKKEMGLICIRDNGIESDDFTYKLADKS |
| | | TVKLPARGTNYLDGVAKLLYSPQSPPKKALAKTQEAKVASKNVNPKIGE |
| | | KYQGVVKNINKHGIFVDFGPKKEGLVRYRNIPMKYKHDSTIVGLDKGDT |
| | | IDVKLLDINEKGYDLKILEDGEFEFEKYKSEKINNTMGSVFKGIDLSFLPK |
| | | K |
| 2108733 | 81 | MKVSKIKHARNAICKRKDSLSIGGRLYDVPNKKNQEQSIENYINNAIEGT |
| | | KKFYTPFKKETVYFYAKKEDISAEKIQDEKVDELKSKIEKEITFRFEKTIT |
| | | RYLDQRNNYLNQTNEDELKRIIKNEWINASKNIKKENEKSEDILKQYFSD |
| | | LFALESNGVSNQADFIYNFLTALFNMKLKRTISKEYVANGKHNNNVDTL |
| | | TKGLMEFSNLAKGKKYNCNSKKFLYVLAAIREDYAKDEQAKSIAKSIKD |
| | | NNVKVQVDKSQKDKLLTLSCAIKKEREYRNIDENLKMFAEQKNNVVLA |
| | | VRDSIPVKNKITVSKDTLFKYVKDFVSECKTQNDKIGHMRCLIILFICGVE |
| | | AYKKAVSCKQITDDFWGNILPVENVYFNSKIDEYLQEMFSLEGQYKTTS |
| | | FLRKKIKDTVNNDIKNNFRTAIAEISGNEFDVYWMKYIQSTASKLLSNID |
| | | KLIKRNDQTYLCKAIYREWTSYLASKYIDMGKAVYNFTIPHNLSDLGND |
| | | IEFGKVLPEYKSGINSFHYEYIKAKENVHRSLAIASMYSVAVFSNNAVSE |
| | | ESRKIPKKEDVLAYSVNDFQKLGRENQVQRILQFFGGKSKWSSISETDYE |
| | | FMFTLKRNLAVARNYSFHFAPSMSEVKEQYQKLALNMFNKEFSRINRLH |
| | | MEKYYNNNVPVFYGKKSIINMLDFLYVNEVVIPEQIPSFNNVFPVKESN |
| | | MLIHQWITSINSNGGYDLPVLTKFRQAFRFALKEIYYKGFLSQPDLYDKFI |
| | | VALKAEKQEIEDNIAFVAEIEKKSAKSNDKLNAINNFSERVDEIKTKDFG |
| | | MLCQQLMVDYEMQNQGRKSVFSTDRKEKAEKRGNKEKYQHFKMILW |
| | | KMIRKAFKKYIDENCDIFGFLKEPSYKENYISKVSIDEFADSWNTPIKMA |
| | | VTEVIKNNVGIRPWYFVAHFLHPKQLNHLKGAVKSYIQFVKDINKRAKS |
| | | IDCSSISEEDMNIAYYEQLLLIFDFVSLFVGRVSNNIEDYFPSCQGKTSKEI |
| | | YIEYINKYIEYDPTDENSVFFDEVNPIVNHAVVFSLMYGADKVLSSTDVH |
| | | YMVSQKEYDTYIQLQKSKSMKNIFSKERCENIEEQKKLNEFQQLKNRVE |
| | | LHDLYSYTEMIIDHLAQLVSYAYMRERDLMYFQLGIQYIRLYHCKVVPE |
| | | GKNKLRCLSNGSDINIKNGAILYQIIAMYNHDLKVFSCDENGNIIEDNKN |
| | | TGDGVGMFLSKNGYGGYLEYINALQLFAINYDGYNGDNEFDRMHKLR |
| | | NSIAHMKYFVGEGRSIIDYYTEIYNGFFRYSSSLRKSIGFIFDNIMMRYFIV |
| | | TNISLDNIEKGKKRKVDIDSMVSDKFTYKYATGEEKLPVHNQQYMDIAR |
| | | NILYYKT |
| 2097776 | 82 | MKFSKESHRKTGVGITENDGAIGLLYRDPMKEEESIEEVVESRVDETTSL |
| | | FNIFTVSQDIKKIIKQKDVESASIEDKEKAILMEEAKNVADLVIVVVRTLK |
| | | SNNNLQTDLFVEKIKKQKKTEVKYTERGIDEALNRFLKKSFRVLPTKNA |
| | | LRELLIYTLIKNQSLSQEQKEKIQKNFIDKLVSEYNKTTVKQNVPKALRN |
| | | QNMVIQPNPNKEGKILAISETPKSKETKTSEKDAFRDFLSEYAVLDENKR |
| | | HELRVKLRRLVVLYFYGPDRVVFCDFDEWKDHESKKSEDVFFADIIKDR |
| | | KVTKNADEKETVNVDATFDSIRTKNISSYRESLSFVNSHKEGFFDNQVLN |
| | | KFWIHHIENEVERIYKHIKPSTPDYKFQIGYLSEKVWKGIINYLSIKYIAEG |
| | | KAVYNYAMTALSNENNSKAFGKIDENYIKGISSFEYERIKAGETLQRECA |
| | | VNIAFAARHLANATVNLDDVNTDFLLLKLDEPSEKEKQRGIKSLVDCAK |
| | | PNVRKNLLQFFGGESIWKDFDFRDIEDIKLLDEFKKMIYSLRNASFHYKT |
| | | ENIDTGSRDTELIAEMFKKDCQNASGIQKNKMFSNNVVKFYREEDIQRL |
| | | LEKLYSKINNRASQVPSFNAVFVRKNFKEYLAKHTNAYDLISDDLKNIW |
| | | ESAVYYLYKEIYYNLFLQDSNSFQYLKDYVDGLDEDKNCTDKKMLRAH |
| | | IDFKNAFKEYAQMGNLSSVCQMIMTEYNNQNQGHRKAISGRSGMGKEL |
| | | KYKHYKIILLNGLRSAFSKYLKENEKDYGFINKPTVNILNLEDFLPTYTSN |
| | | QYNDLVECVRSDFELQKWYVVGRLLNLKQVNQLIGSFRSYTQYVNDVK |
| | | RRAELTGNPIRNMDVSVDVENIVGVLDVCTKLNGMTSNILEDYFDNKD |
| | | DYARFVAMFVDFGLEEDSDYADAELAAFCNQEINGQKIGIYHDGTNPIIN |
| | | RNVILCKLYGATEIISKSEVKKVDLSTIREYFKLANKIKQYKVSGVCRDK |
| | | VEQLNVREYQEIKNRVELRNIVEYSEIINELQGQLINWGYLRERDLMYFQ |
| | | LGFHYLSLHNNEEKPNGYDNAGDEYTGAILYQIVAMYTNGIDLIDVNGK |
| | | SKGKENLPTGAKVNLFGSYSKEMLGLAKKEKDPIYIAGLELFEYVKEHN |
| | | QCVDLRNYIEHFHYYAKHNRSMLDIYSEVFDRFFTYDMKYAKNVPNM |
| | | MYNILLQHLVVAMFDFTSGEKKYGDSYDLLKKRASISIRKPNGLASEKFI |
| | | YNLENGKKVIKLTARGRAYLEDIATIIYYPDDVPEEIIKDPIEDDKDAKGK |
| | | DKKVKIQNDGKKRTFNNHNKKNNHGYRSKQPPKKKDYYSKRNH |
| 2086789 | 83 | MKIAKIRNSRSGISVAENKNEEQHSRQSFLYYAPGKDGKVRLESKDLEQ |
| | | HVMDCNRKAQNLYKVITPLEADFSRNKDDKEVFEDLAENFNAAINAAL |
| | | KSARTVKSNSESDLEIKLKQRVKKETEFILNLGENKIKMKKQEMQIQEMI |
| | | SELVLRRLKKTLRKEVVIADGRRMQMTEIVNSLMCAICLYGNVESFDPV |
| | | VLNAFFEKVDSDYMKYKQIKQIVKSIESQSVKVKVTEENGKYLLIPANA |
| | | EHKKKKYVFEFMKKYAVAEEKEKRELTGHIQELIGLYFCGESGYQKAG |
| | | NSFADMIENHDCISEEIKEVLDSVETLENLSKQKREEKKEIREELERNKAA |
| | | GKENKKGGKKEDKKSQNDELKETIKKLDSEIQKLSSEIRTKREELKNTTE |
| | | RVMSVHYRAAVEYLADKVGETILGQIKEKKVCACADLYWIDYIDDTVK |
| | | KLLVDSKNKGNYRYTTGFLANHIWKEWTQYISGKYIELGKGVYHFAVP |
| | | DLSGAVDGETVSICEVKPKYRSGISGFDYERIKAEESLEREMEEYILFAVN |
| | | NFARAVAPEEEREREGHEDVLLMKTLSEIEEGSNTQSKNKQDKKNNSKK |
| | | AIVLYADADRRILQFFGGQSRFREKEDSLINLYSGEDLYKEIRNELYAIRN |
| | | VAFHYTTKADKDKVRKHEMAESFFEEELADVSEVFRKKYYSNNVWKF |
| | | YSTEVINSLMEKIYSGRKYRAAQIPAFNKIISRPALPQIMERFINGTPLKRI |
| | | KNNPDKNVVIQYWSALFFVLKELYYYDFLQEQENLEDNIKERFFRALER |
| | | KSGQAEEDKKKAWESFRNRIEEIGKTSSFGEICQGLMVEYMLQNNDSSM |
| | | VRKKNIYKKTSGRDTQIYKHYRTLLYICIRDAFTEYLKEKWPELRKPILL |
| | | TKEWSEEEFCQTDGMKHLSLYDHLKEIFYSTVSGPSWYIAAHFINPKYLN |
| | | HLIGSIRNYLQFTEDIERRAVSLGDSTDIEIAEKNLRYHNTLEVLEFVIQFC |
| | | GKTTKVMEDYFESNQAYAAYLSDFVDYKTSKHVSDIETALHIFCEQKFK |
| | | IDGKECMTGIYYDGGNLIPNRNIIRANMYGNVSCLKNSMKKITVAEIRQ |
| | | MYTDKAKLDTVLKEGVCRTRQEQEKLREFQNEKNRIELLDLAIYTEILN |
| | | DMQAQLIGWSYLRERDLMYYQLGYYYTKLFWTDAIGKEDVRRKLVGD |
| | | EVHIEDGAILYQILAYNSYDLPIISNIGEAAYFMKSEESIGGKAIGAFCKNF |
| | | INAVSIYLEGLELFENTDEHDNIIKTRNYIEHFKYFIRSDRSMIDLYSEVYD |
| | | RFFRHDYKLKKSVPTVLINILARNFMDAYIYMELGCKTVGTKKTQEHKA |
| | | TQIAFTDKGIESTALTYTIQPDPISKDNEKKEEKKKEEIPAHSEIFLHQFRSI |
| | | LEYRKGNGNN |
| 2070794 | 84 | NDAVIEGDFDEWQDHAKRKSDSGVFVEKVVLTKKDRNGKTIESLNVDG |
| | | TKDAIRKKNISCYRESVAFAKEKKELFFEDDVLNKFWIHHIESEVERIYRS |
| | | INNTTGNYKFELGYLSEKVWKGIINYLSIKYIAEGKAVYHYCMNALSDE |
| | | NKAFGVLDERYKNGITSFEYERIKAEETLQRECAVSISFATNHLANATVN |
| | | LDEKNSDFLLIKNLEDVKKPHVVKNILQFFGGASTWEDFDFSGYDEVEV |
| | | LAGFKEAIYSLRNTTFHFDTKNKDQVKWNTEFIGAMFAHDCKMARKVQ |
| | | KDKMYSNNVSDFYSEENIKKVLETLYSDIHERASQVPSFNKVFVRKNFP |
| | | VYLTEQKLTPKFESAEMQKWQSAVYYLYKEIYYNAFLQDKKSYELLKN |
| | | FVHKLPENEKDDKGKRTQESAANANFKEAFSEYEKEASSLSAICQLILTE |
| | | YNNQNKGNRKAISARTNKADELIFQHYKMILLKGLEAAFTEYVKQNTV |
| | | YDFIKNPTVLQNVPSQDDFLADYKTLQFEGLSKEVNATPDLQKWYMVG |
| | | RLLNPKQLNQLIGSFRSYEQYVNDVARRAKQTKNPILDSEFSVDVHGILS |
| | | VLSVCTSLSGVVSNNWKDYFDSEDAYAEYIAKFVDFGLKDNSDLANAA |
| | | LAQFCKKIGIYHDGTNPIVNRNVILCKLFGSSDILSKAGILKVDETVIKKY |
| | | KTQEKETTGYRTTGICKDKAEQEKLKKFQELKNLVELRNIVEYLEILNEL |
| | | QGQLINWSYLRERDLMYFQLGFHYLCLQKNGKDISGYNKAGDVTGAIL |
| | | YQIVAMYTNGIPMIDKDGKSDDKGELQTSGKLNRFASYSETVNGDKWN |
| | | IYLSGLELFENIDEHDQCVKLRNDIDHFHYFSEQNRSMLDYYSEVFDRFF |
| | | TYDMKYTKNVANMLYNILLSHLVVATFGFGKGEKEVGEGKNKATKPR |
| | | ASIVLKTNGLASEKFTYKFSDGKTISLCARSNDFLKDVAALLYYPDKAPA |
| | | DVCRDTTVEDNRAKPENSKRDVNNFNRGNGSKNHSDKLRGKYED |
| 2065400 | 85 | MKISKESHRKTGVGIDDFGIKGLMYIDSLGKNDKKDSIATIVKRRADSTK |
| | | RLYKLFGTKNSNVSKEAKYVAEIVNDSLKKIKLYPREYFDGKKKKERKD |
| | | STLRAGTLIKAIREEIKAKESTIKEKCKGEVVNNPLNISKNDIEQAVDSLL |
| | | KKSFREKNEKDAIKLVLYSAFSIERKRLSDKELENITKYFINKLVDDYNK |
| | | TILQKEIPIAIENQNMVVQPRIEDGTLVVAEIEKSSNRKKAEKAAFREFLS |
| | | DYASLNENDRNEMLRKVRRLVDLYFYGGDFVRKDDFNVWSIHEERKK |
| | | CKEKFIEQKNNNKNDEKPSKAGNIDEIKDEIRTENLHRYMDSLKAIKEDN |
| | | SCYFENDILNKFWIHHIENEVERIYGHLKNDTGDYKFQLGYLSEKVWKG |
| | | IINYLSIKYIAEGKAVYHYAMGALASDEGAKIFGRLDERYLNGISSFEYER |
| | | IKAEETLQRECAVNVAFAANHLANATVQLDDINTDFLLLKNNELSEDNK |
| | | KKGIKSLRDCATAKPNTLRNILQFFGGMSAWDDFDFGGREAIELLNDMK |
| | | EMIYSLRNMSFHFKTENVNEGKWNTELIGRMFAHDCEVASLVQKNKLY |
| | | SNNVAMFYGSSDLAKLLDKLYATYNERASQVPSFNTVFVRKNFPEYLKE |
| | | QKITPTFSSDDTLKWQSAVYYLYKEIYYNAFLQDKESFNLLKRYVEKLD |
| | | EHCQDKNQAGANRDFKEAFGIYSKSGSLSATCQMIMTEYNNQNKGNRV |
| | | GISGRTNSDDKLIFQHYKMILFDGLRAAFFEYLKKNGEVYDFIKTPKLVT |
| | | TLVDADVFLGDYKSHQYDSLVENIKTKPELQKWYITGRLLNPKQVNQLI |
| | | GTFRSYVQYVNDVMRRAKQVGNPLTDATISIDVDNIIKVLDICTKLNGV |
| | | TSNKIDDYFDSYDEYAKYLSKFVDFGLRGDKDLANAQLAAFCEREVEG |
| | | QKIGIYHDGTNPVLNRNVVLCKLYGASDVVAQAGILPVDFEIIKKYMQL |
| | | SGKIKEYQLTGICKTKQEQLKLKKYQELKNMVELRNIVDYAEIIDELQGQ |
| | | LINWGYLRERDLMYFQLGFHYLSLNNTAPKPEGYEMAGDYNGAILYQI |
| | | VAMYTNGLSLIDADGKRKRNAKASTRFKIESFCSYSKSLRNVPGDTKEEI |
| | | DPIYHAGLELFENIKEHDQCVYLRNYIEHFHYYAKMDRSLLDIYSEVFDR |
| | | FFTYDMKYAKNVPNMLYNILLQHLVVPSFEFHSGEKKLGDHNSLSKKR |
| | | ATISLKSENGLASEKFTYKIGDKKNPIKLSARSNDYLSDVAAILYYPDTAP |
| | | AQVVRDTVKEDNSAKEESKKSKDFAELPNNKNNNYHGRNNGYKNQKR |
| | | DYSK |
| 1630 | 86 | MKISKVNHHKSAVSLNENDIKGILYVDPHISKNTELEKYIAERISASQNLF |
| | | SLFKNVKNKDIKKMCSKIDKTIKNKNGNVTASDLININVKKNDADGSEE |
| | | NIENLKKNIRKCVNVNTRDFIARDKRLKKALEKLLLNMRNIEKIDNGDE |
| | | DIKRLALAINKAYLKDASGIKKSIENQNLSVQLSKDNEHFEVYPVTNLQN |
| | | TNSVKKNEKEAFKHFMSDYARIDKHKRDELLLKLRRLNVLYFYGYDEA |
| | | KNSLKYPEDIFADHEKRKCDKVKFVDKNILNEELLRLHNASIESIEKDNS |
| | | LFFDDINLNRFWIRFIGNYTQKQFKKFLDSEDDFKKNRGYICEKAWKEIL |
| | | CFLSGKYIAEGKMVYQFAMDKIASNESEYDFGIIPERYKDGIKSFDFEMIS |
| | | ANESIKRDIAVSVTFAVNNIARSVIDDKKAKNNYDLLSIDDEKLVSIMYD |
| | | NEALKRNIYQFWGGMSKWKSFEDNNVFCSTKDYYHNLVIDIRNILANV |
| | | RNENFHFKTSRYNAKKIDIDRLSKIFEFEANNASKVYRDKFYSNNLWEFY |
| | | SDADMENALNILYSSYNYKASQIPAFGNVISRGKLNDFLVNDIKMTINYR |
| | | DSETTERCRNAIYYLLKEVYYRGFLASNDVYEYFVNAIKKVEDEKKNFN |
| | | FKDKQFQSIDDYVKRINELENNINTSNGKDKIAKICQFIMTENNQQNAGR |
| | | KKVDTSKTKKNNKRIFEHYKMLLYKTLKYAYLDYLNKSAFAFIKNPKN |
| | | DQMRRKDIAGTEFLPNWNAKTYISLVEKFKTENELQKMYIVAKLLPPRQ |
| | | LNRLIGEFKNYKSYTEDIERRANSNANILLVNKLYTDAVYNDIIQVLEMC |
| | | MVQSGVITNKINDYFKDENAYSDYLRRYIDKDIENFELYKDGKNFILNR |
| | | NVVLSKIYGAHEVLENTIEKISQKDIEDYKNALINVEEFRKTGKCESSEEQ |
| | | KNVNIYQRIKNRVTLANVLTYSEIINSLLGQLVGWSFLRERDLMYFQLGF |
| | | HYECLQNDEEKMPTYNMIEVMQSDNTVLKINNAILYQIAAMYINGMPIY |
| | | ACDNNKEFSTVSTGLKIKMFCEYTLNAFNDKSRKEDYYNAGLEIFEDVK |
| | | EHDNIANLRNHIDHFKYYSIRKDMPNEYAKSLLDLYSETFDRFFTYDNKL |
| | | RKSVPVVFENILRRYFVILSLKFDTAKKNIGKNNEKDMAHFTVTPRSDSF |
| | | TYKFKEKNPNDKDNNDNVRDEQFLVNIAKILYYPNEVPQDVLENINKKD |
| | | NNKNNKKNNKNKTKSKDFTYKPFKDIKL |
| 1626 | 87 | MKISKVDHNKTAVGNMDNMPIKGFIYKDPKRVENGTSVDINKRIKDAR |
| | | RTFESIYSVFDEKKFNKDTQDFNLVKQFNKDIRNVLKNSKGNIGFNELIN |
| | | GLNKFDSIKYEPDNIIYSGLKQSFQTEKIIKSIRILIRYVCENIKDINTDEKK |
| | | LIENSFIGKIIKDYQKFDKCVHIPKAISNQNMVVQIDNDTRKLNLAKIPPH |
| | | PESKHDDNSREREAFKRFLSGYSNLDDSVRHNIRVRLRRLVDLYFYGIEA |
| | | VKLEDFNEWKDHEEKKARNDLFVDKQEITVKKESEGKKTEVDADLIKD |
| | | TVRKKNIIRYRNTIKIASDNPTIFFDDQDVNVFWIHFIEKEVEKLFSKIKNN |
| | | NDFVLKIGYVSEKVWKSMINYISIKYIALGKAVYHCALKDLDITDTSNLN |
| | | IDLTKIDDKYLNGISSFHYELIKAEEDLQRDVSESVAFAANHLAHATISNI |
| | | NSKDDFLTLTYEDLKKKAYPNTMKNIMQFFGGYSKWKSFFEKNHSEDY |
| | | DDIVFLRDIRNIIYSLRNSSFHFTTKKIDNDSWNTELIGNMFEKDCEIESTV |
| | | LKNKLYFNNLPMYYADKPLENVLHRLYDSCHERASQVPSFNKVFIRKNF |
| | | ASILLKYNYNVVFNDDLDMLNWQSAIFYLFKEIYYNDFLQNDSSLSLFLR |
| | | YVDDLKIEKDDRGKTTKSTRPNEDFKNAINFFKNDNTTLSSLCQQIMTEY |
| | | NRQNDTGVKKRNYNVEKKHPKIFQHYRMILFDGLRHSFMRYVQENEKI |
| | | FGFIKKPNKKSEGMSSVTEFLPNYFSRRYINLIETVKTNPELQKWYILGRL |
| | | LNPRQVNLFIGDIRRYVQYINDILRRAKENGNNINNYIMPIDYKHIIEILEI |
| | | CTKLNGVVSNVVTDYFTDEDDYSSHIGKYIDFELENSAVTLRDFCNQIYK |
| | | GKRIGIYYDGVNPIVNRNVVLCKLFGITDILDKNKKIKKVTKDVLYKYLD |
| | | QQDKLVKYRKDGLCTNIAEQEFVNQYQWLKNRIELRDIVEYTEILNELH |
| | | GQLIKWVYLRERDLMYFQLGFHYLCLNNNSPKPKEYVEINVGGVSLYG |
| | | AILYQIISMYTNGMNLYYIYEGSDYISKMAVITGHRTGKKLRYHNIELNT |
| | | YEINKIRDEKFNGTESIGSKIKYFLRYGDGIGHDKGYGEQLYYTGLEIFEN |
| | | LSEHDNIANLRKYIEHFRYFSQMDRSMLDIYSEVFDRFFSYDSKLRKNVI |
| | | NMMYNILLSHQVISKYQFGTSEKMIGTGEKSLKQQAACFMFKEKNGIDS |
| | | DKFTYKYKQDGQNANKKTPQMLHARNTEFLMNVANLISYPKEVANDV |
| | | VGCERENKKVRINNKVKNTAPVNN |
| | 88 | MKVTKVDGISHKKYIEEGKLVKSTSEENRTDERFNDIEKKLSELLSIRLDI |
| | | YIKNPDNASEENRIRRENLKEFFSNKVLHLKDGILYLKDRRELKNAVQN |
| | | KNYSEQDISEYDLKNKNSFSVLKKILLLKLNENVKEEKIKFNSEELKEVF |
| | | RKDIKKKLNKINSLKYSFEKNKANYQKINENNIEKVEGKSKRNIIYDYYR |
| | | ESAKRDAYVSNVQEAFDKLYKNSEEDIAKLFLEIENLTKHEKYKIREFYH |
| | | NEIIGRKKENFAKIIYEEIQNVNKEENMKELIEKVGNPDMSELKKSQVFY |
| | | KYYLDKEELNDENIKYVFCHFVEIEMSKLLKNYVYKKPSNISNDKVKRIF |
| | | EYQSLKKLIENKLLNKLDTYVRNCGKYSFYLQDGEIATSDFIVGNRQNE |
| | | AFLRNIIGVSSVAYFSLRNILETENEDFYEIENDITGRMRGKESNIVSGEVD |
| | | KIYNENKQNEVKENLKMFYSYDFNMDNKNEIEDFFANIDEAISSIRHGIV |
| | | HFNLDLDGKDIFAFKNIVPSEISKKMFQNEINEKKLKLKIFRQLNSANVEN |
| | | YLEKYKILKYLKNTRFEFVNKNIPFVPSFTKLYNRIDDLKGKNSLKIYWK |
| | | IPKDKEEKDAQIYLLKNIYYGEFLNKFVSNKNGNFFKISKEIIELNKNDKR |
| | | NQKTGFYKLQKFENIEETIPKEYLAIIQSLYMINAGNEQDEEEKDTYIDFI |
| | | QKIFLKGFMDYLNNNNRLKLIEINNDETNKILAEKKQEFDKILKKYEQNN |
| | | RNLIEIPYEINEFVREIKLKEIGKILKYTERLNMFYLILKLLNHKELTNLKG |
| | | SLEKYQSANKEETFSDQLELINLLNLDNNRVTIEDFELEADEIGKFLDENS |
| | | GNKVKDRKELKKFDTKENTGEYNKIYFDGENIIKHRAFYNIKKYGMLNL |
| | | LEKISDEAKYKISIEELKKYSNKKNEIEDEKNHKMQENLHRKYARPRKD |
| | | EKFTDEDYESYKQAIKNIEEYTHLKNKVEFNELNLLQGLLLRILHRLVGY |
| | | TSIWERDLRFRLKGEFPENQYIEEIFNFDNKKNVKYKSGQIVEKYIKFYKE |
| | | LHQNDEVKINKYSSANIKVLKQEKKDLYIRNYIAHFNYIPHAEISLLEVLE |
| | | NLRKLLSYDRKLKNAVMKSVVDILKEYGFVVTFKIGAESNDKKIEIQSLK |
| | | SEEIVHLKKLKLKDNDKKKEPIKTYRNSKELCKLVKVMFEYKMEN |
| | 89 | MKVTKVDGISHKKYIEEGKLVKSTSEENRTSERFNDIEKKLSELLSIRLDI |
| | | YIKNPDNASEENRIRRENLKEFFSNKVLHLKDGILYLKDRRELKNAVQN |
| | | KNYSEQDISEYDLKNKNSFSVLKKILLLKLNENVKEEKIKFNSEELKEVF |
| | | RKDIKKKLNKINSLKYSFEKNKANYQKINENNIEKVEGKSKRNIIYDYYR |
| | | ESAKRDAYVSNVKEAFDKLYKNSEEDIAKLFLEIENLTKHEKYKIREFYH |
| | | NEIIGRKKENFAKIIYEEIQNVNKEENMKELIEKVGNPDMSELKKSQVFY |
| | | KYYLDKEELNDENIKYVFCHFVEIEMSKLLKNYVYKKPSNISNDKVKRIF |
| | | EYQSLKKLIENKLLNKLDTYVRNCGKYSFYLQDGEIATSDFIVGNRQNE |
| | | AFLRNIIGVSSVAYFSLRNILETENEDFYEIENDITGRMRGKESNIVSGEVD |
| | | KIYNENKQNEVKENLKMFYSYDFNMDNKNEIEDFFANIDEAISSIRHGIV |
| | | HFNLDLDGKDIFAFKNIVPSEISKKMFQNEINEKKLKLKIFRQLNSANVEN |
| | | YYEKDVIIKYLKNTKFNFVNKNIPFVPSFTKLYNKIDDLRGKNTLKFYWK |
| | | IPKDKEEKDAQIYLLKNIYYGEFLNKFVKNKSKDFFKITDEVIKINKQDK |
| | | RNQKTGYYKYQKFENIEKTVPVEYLAIIQSRDMINNQDKEEKNTYIDFIQ |
| | | QIFLKGFIDYLNKNNLKYIENNNNDIFSRIKIKKDSKERYDKILKNYEKNN |
| | | RNLKEIPYEINEFVREIKLKEIGKILKYTERLNMFYLILKLLNHKELTNLK |
| | | GSLEKYQSANKEETFSDQLELINLLNLDNNRVTIEDFELEADEIGKFLDEN |
| | | SGNKVKDRKELKKFDTKENTGEYKKIYFDGENIIKHRAFYNIKKYGMLN |
| | | LLEKISDEAKYKISIEELKNYSNKKNEIEDEKNHTMQENLHRKYARPRKD |
| | | EKFTDEDYENYKRAIKNIEEYTHLKNKVEFNELNLLQGLLLRILHRLVGY |
| | | TSIWERDLRFRLKGEFPENQYIEEIFNFDNKKNVKYKSGQIVEKYIKFYKE |
| | | LYQNDETKINKYSSANIKVLKQEKKDLYIRNYIAHFNYIPHAEISLLEVLE |
| | | NLRKLLSYDRKLKNAVMKSVVDILKEYGFVVTFKIGAESNDKKIEIQSLK |
| | | SEEIVHLKKLKLKDNNKKKEPIKTYRNSKELCKLVKVMFEYKAEN |
| | 90 | MKVTKVDGISHKKYIKEGKLVKSTSEENRTDERFNDIEKKLSELLSIRLD |
| | | MYIKNPDNASEENRIRRENLKEFFSNKVLHLKDGILYLKDRRELKNAVQ |
| | | NKNYSEQDISEYDLKNKNSFSVLKKILLLKLNENVKEEKIKFNSEELKEV |
| | | FRKDIKKKLNKINSLKYSFEKNKANYQKINENNIEKVEGKSKRNIIYDYY |
| | | RESAKRDAYVSNVQEAFDKLYKNSEEDIAKLFLEIENLTKHEKYKIREFY |
| | | HNEIIGRKKENFAKIIYEEIQNVNKEENMKELIEKVGNPDMSELKKSQVF |
| | | YKYYLDKEELNDENIKYVFCHFVEIEMSKLLKNYVYKKPSNISNDKVKR |
| | | IFEYQSLKKLIENKLLNKLDTYVRNCGKYSFYLQDGEIATSDFIVGNRQN |
| | | EAFLRNIIGVSSVAYFSLRNILETENEDFYEIENDITGRMRGKESNIVSGEV |
| | | DKIYNENKQNEVKENLKMFYSYDFNMDNKNEIEDFFANIDEAISSIRHGI |
| | | VHFNLDLDGKDIFAFKNIVPSEISKKMFQNEINEKKLKLKIFRQLNSANVF |
| | | RYLEKYKILNYLKRTRFEFVNKNIPFVPSFTKLYSRIDDLKGKNSLGIYW |
| | | KTPKTNDDNKTKEITDAQIYLLKNIYYGEFLNYFMSNKNGNFFEISKEIIE |
| | | LNKNDKRNLKTGFYKLQKFEDIQEKIPKEYLANIQSLYMINAGNEQDEE |
| | | EKDTYIDFIQKIFLKGFMTYLANNGRLSLIYIGSDETTSLAEKKQEFDKFL |
| | | KKYEQNNNLIEIPYEINEFVREIKLKEIGKILKYTERLNMFYLILKLLNHKE |
| | | LTNLKGSLEKYQSANKEETFSDQLELINLLNLDNNRVTIEDFELEADEIG |
| | | KFLDFNSGNKVKDRKELKKFDTKENTGEYNKIYFDGENIIKHRAFYNIK |
| | | KYGMLNLLEKISDEAKYKISIEELKKYSNKKNEIEDEKNHKMQENLHRK |
| | | YARPRKDEKFTDEDYESYKQAIKNIEEYTHLKNKVEFNELNLLQGLLLRI |
| | | LHRLVGYTSIWERDLRFRLKGEFPENQYIEEIFNFENKKNVKYKSGQIVE |
| | | KYIKFYKELHQNDEVKINKYSSANIKVLKQEKKDLYIRNYIAHFNYIPHA |
| | | EISLLEVLENLRKLLSYDRKLKNAVMKSVVDILKEYGFVVTFKIGAESND |
| | | KKIEIQSLKSEEIVHLKKLKLKDNDKKKEPIKTYRNSKELCKLVKVMFEY |
| | | KMEEKKSEN |
| | 91 | MKITKIDGISHKKYIKEGKLVKSTSEENKTDERFNDIEKKLSELLSIRLDT |
| | | YIKNPDNASEENRIRRENLKEFFSNKVLHLKDGILYLKDRRELKNAVQN |
| | | KNYSEQDISEYDLKNKNSFSVLKKILLLKLNEDIKEEKIKFNSEELKEVFR |
| | | KDIEKKLDKINSLKYSLEENKANYQKINENNIKKVEGKSKRNIFYNYYK |
| | | DSAKRNDYINNIQEAFDKLYKNSKEDIENLFFLIENSKKHEKYKIRECYH |
| | | NKIIGRKKENFATIIYEEIQNVNKEENMKELIEKVGNPNVSELKKSQVFY |
| | | KYYLNKEKLNDENIKYVFCHFVEIEMSKLLKNYVYKKPSNISNDKVKRI |
| | | FEYQSLKKLIENKLLNKLDTYVRNCGKYSFYLQDGEIATSDFIVGNRQNE |
| | | AFLRNIIGVSSVAYFSLRNILETENEDFYEIENDITGRMRGKESNIIKNNKG |
| | | EISGEVDKIYNENKQNEVKNNLKMFYGYDFNMDNKNEIEDFFANIDEAI |
| | | SSIRHGIVHFNLDLDGKDIFAFKNIVPSEISKKMFQNEINEKKLKLKIFRQL |
| | | NSANVFRYLEKYKILNYLKRTRFEFVNKNIPFVPSFTKLYSRIDDLKGKN |
| | | SLGIYWKTPKTNDDNKTKEITDAQIYLLKNIYYGEFLNYFMSNKNGNFF |
| | | EISKEIIELNKNDKRNLKTGFYKLQKFEDIQEKIPKEYLANIQSLYMINAG |
| | | NEQDEEEKDTYIDFIQKIFLKGFMTYLANNGRLSLIYIGSDETTSLAEKKQ |
| | | EFDKFLKKYEQNNNLIEIPYEINEFVREIKLKEIGKILKYTERLNMFYLILK |
| | | LLNHKELTNLKGSLEKYQSANKEETFSDQLELINLLNLDNNRVTIEDFEL |
| | | EADEIGKFLDFNSGNKVKDRKELKKFDTKENTGEYNKIYFDGENIIKHRA |
| | | FYNIKKYGMLNLLEKISDEAKYKISIEELKKYSNKKNEIEDEKNHKMQEN |
| | | LHRKYARPRKDEKFTDEDYESYKQAIKNIEEYTHLKNKVEFNELNLLQS |
| | | LLLRILHRLVGYTSIWERDLRFRLKGEFPENQYIEEIFNFENKKNVKYKSG |
| | | QIVEKYIKFYKELHQNDEVKINKYSSANIKVLKQEKKDLYIRNYIAHFNY |
| | | IPHAEISLLEVLENLRKLLSYDRKLKNAVMKSVVDILKEYGFVVTFKIGA |
| | | ESNDKKIEIQSLKSEEIVHLKKLKLKDNDKKKEPIKTYRNSKELCKLVKV |
| | | MFEYKMEEKKSEN |
| | 92 | MKVTKVGGISHKKYTSEGRLVKSESEENRTDERFNDIEKKLSALLNMRL |
| | | DMYIKNPSNTEKENQKRIGKLKKFFSNKMVYLKDNTLSLKNGKKLENIS |
| | | ETDISEYDVRDKKNFAVLKKIYLLKLNENVKEEKIKFNSEELKEVFRKDI |
| | | KKKLNKINSLKYSFEKNKANYQKINENNIEKVEGKSKRNIIYDYYRESAK |
| | | RDAYVSNVKEAFDKLYKNSEEDIAKLVLEIENLTKLEKYKIREFYHNEII |
| | | GRKKENFAKIIYEEIQNVNKEENMKELIEKVGNPDMSELKKSQVFYKYY |
| | | LDKEELNDENIKYVFCHFVEIEMSKLLKNYVYKKPSNISNDKVKRIFEYQ |
| | | SLKKLIENKLLNKLDTYVRNCGKYNYYLQDGEIATSDFIARNRQNEAFL |
| | | RNIIGVSSVAYFSLRNILETENEDFYEIENDITGRMRGKESNIIKNNKGEVS |
| | | GEVDKIYNENKQNEVKENLKMFYSYDFNMDNKNEIEDFFANIDEAISSIR |
| | | HGIVHFNLDLDGKDIFAFKNIVPSEISKKMFQNEINEKKLKLKIFRQLNSA |
| | | NVFRYLEKYKILNYLKRTRFEFVNKNIPFVPSFTKLYSRIDDLKGKNSLGI |
| | | YWKTPKTNDDNKTKEITDAQIYLLKNIYYGEFLNYFMSNKNGNFFEISK |
| | | EIIELNKNDKRNLKTGFYKLQKFEDIQEKIPKEYLANIQSLYMINAGNEQ |
| | | DEEEKDTYIDFIQKIFLKGFMTYLANNGRLSLIYIGSDETTSLAEKKQEFD |
| | | KFLKKYEQNNNLIEIPYEINEFLREIKLKEIGKILKYTERLNMFYLILKLLN |
| | | HKELTNLKGSLEKYQSANKEETFSDQLELINLLNLDNNRVTIEDFELEAD |
| | | EIGKFLDFNSGNKVKDRKELKKFDTKENTGEYNKIYFDGENIIKHRAFYN |
| | | IKKYGMLNLLEKIADKAGYKISIEELKKYSNKKNEIEDEKNHKMQENLH |
| | | RKYARPRKDEKFTDEDYESYKQAIKNIEEYTHLKNKVEFNELNLLQGLL |
| | | LRILHRLVGYTSIWERDLRFRLKGEFPENQYIEEIFNFENKKNVKYKSGQI |
| | | VEKYIKFYKELHQNDEVKINKYSSANIKVLKQEKKDLYIRNYIAHFNYIP |
| | | HAEISLLEVLENLRKLLSYDRKLKNAVMKSVVDILKEYGFVVTFKIGAES |
| | | NDKKIEIQSLKSEEIVHLKKLKLKDNDKKKEPIKTYRNSKELCKLVKVMF |
| | | EYKMEEKKSEN |
| 3610137 | 93 | MENQTKTGKGIYYYHNSIEDKHYFGGFLNLAQNNIDNVIEEFRIRLNLPA |
| | | YSIKNPVKKLSAIDIISNYFGQQSISYTDWERGVNILKEYLPVIDYLDLPVT |
| | | DKEFENIDLKAKEKAKRDYFRKNLLGLLGTINNLRNFYTHHYHPLITIDE |
| | | NIAQFLDKTLFDVCMEVRKQKMKKDKTKQALKDGIAEELKKLIELKKA |
| | | ELKEKKIKTWNVEKNVEGAVYNDAFAHMIYKNKAEVTVLKDVHKTKV |
| | | PTEKISGDLKVDFSMSSLIFLLSMFLSKKEIEQFKSNIERFKVKVIGESEIQE |
| | | ISKFNNSLKFMATHWVFSYLAFKGLKHRIKSVFDKETLLMQMIDELNKV |
| | | PDEVYQTLSKEQQKEFLEDINEYVQDNEENRKSLEGSTIVQPLIRKRYDD |
| | | KFNYFAIRFLDEFANFPTLKFFVTVGNFLHDKKNKQIKGSTVTSERLIKEK |
| | | INVFGKLSEIVKYKSDYFSKKDINDETGWELYPNPSYLLNGNNIPVYIDL |
| | | MGKGEDAKKCQIEINQLRKKTNPEKPRNLRASKEEIINIIYSKNKNIKHGE |
| | | PTALLSANELPALLYELLINKKTGEDIENIIVNKIVQQFQTVSKYDNSINLP |
| | | NSKITKKLKKADSAETIINTDKIIHAIKKEIEISDNKLELIRNNRNDYYKKS |
| | | RKYIFYLKELGQEATWLAYDIKRFMPENARKNWKGIYHNELQKYLAFY |
| | | DRNKNEAINLLSEFWDLNNDHFIGQELKNAFHEKYFDRFYESYLNARKN |
| | | ILTGFLELIKNLKNEPVILKKGLKEVFRIFTKRLYVINPMNIQKEQLLSKPI |
| | | CLPRGIFDEKPTFINDTKFEDNPSLFADWYQYTLKDTHQFQEFYNIQRDY |
| | | KELFEKYKAVDNEFIKNKKKLNSEDMFSLFKYKEDMKIRRIKAQDLFIKL |
| | | MVDELFNVIFSQKINLNLKEFYQTSDERYTNRLVAIEQSKRKKGDNSEN |
| | | KLNENFIWNKTVAVSLFNGQIYEPAVKLKDIGKFRKLENDEKVIQLLEY |
| | | DKSKVWNKLGIEDEIENSPDSYERIRREKLLKEIQEFETYILKKENFDGVN |
| | | HPEKFEQKQKTEDGKGSPNFKMYMVHGVLNKINALNNDDYKLLSKKEI |
| | | DNIPVSEIMQLPVLVQKAYLLIIIRNKFVHNQFPDTHIFNLAKQFYPKINEE |
| | | TYAKYYFDVCTNIILEFKNQYNVL |
| | 94 | MKVTKIDGLSHKKFEDEGKLVKIEDASQKNETLERLENLKGIKLGSYIKN |
| | | PDKTKNKDNKKRRKGLKEYFSEITLRKENEKYVLLKGKKLKKINNDIKD |
| | | TDIKAKDKKEEVFDILKEILKLNLLANDAEEKIQFDSIKLKNVFGKDFAK |
| | | KELQIKSIEESLEKNRADYRKEFIEIENHKYGNVKGKNKRSRIYGYYKKS |
| | | ENHKKFEDNIREAFEKLYTEENIKKLYSKIEEVLKKTHLKSIVREFYKNEII |
| | | GESEFSKKNGDGISILYNQIKDSIKKEENFIEFIENIGNLELKDLTKSQIFYK |
| | | YFLENEELNDENIKFVFCYFVEIEVSDLLKGNVYKASKIYENKIKNIFEYD |
| | | KLKKLVINKLENKLNNYVRNCGKYNYHMENGDIATSDINMRNRQTEAF |
| | | LRSMIGVSSFGYFSLRNILGVNDDDFYEIEEDLTKDERKKESSTLKKVKE |
| | | DIASKSIFEKVVDKSFQKKGIYNIKENLKMFYGDSFDDADEDELKQFFVN |
| | | MLNGITSIRHRVVHYNMNTNSENIFNFSDIEVSRLLKNIFEKETDKRELKL |
| | | KIFKQLNSAGVFDYWESWKIKKYLENIEFKFVNKNIPFVPSFTKLYSRIDD |
| | | LKNSLGIYWKTPKTNDDNKTKEIIDAQIYLLKNIYYGEFLNYFMSNNGNF |
| | | FEISKEIIELNKNDKRNLKTGFYKLQKFEDIQEKIPKEYLANIQSLYMINA |
| | | GNQDEEEKDTYIDFIQKIFLKGFMTYLANNGRLSLIYIGSDEETNTSLAEK |
| | | KQEFDKFLKKYEQNNNIKIPYEINEFLREIKLGNILKYTERLNMFYLILKL |
| | | LNHKELTNLKGSLEKYQSANKEEAFSDQLELINLLNLDNNRVTEDFELE |
| | | ADEIGKFLDFNGNKVKDNKELKKFDTNKIYFDGENIIKHRAFYNIKKYG |
| | | MLNLLEKIADKAGYKISIEELKKYSNKKNEIEKNHKMQENLHRKYARPR |
| | | KDEKFTDEDYESYKQAIENIEEYTHLKNKVEFNELNLLQGLLLRILHRLV |
| | | GYTSIWERDLRFRLKGEFPENQYIEEIFNFENKKNVKYKGGQIVEKYIKF |
| | | YKELHQNDEVKINKYSSANIKVLKQEKKDLYIRNYIAHFNYIPHAEISLLE |
| | | VLENLRKLLSYDRKLKNAVMKSVVDILKEYGFVATFKIGADKKIGIQTL |
| | | ESEKIVHLKNLKKKKLMTDRNSEELCKLVKIMFEYKMEEKKSEN |
| | 95 | MKVTKVGGISHKKYTSEGRLVKSESEENRTDERLSALLNMRLDMYIKNP |
| | | SSTETKENQKRIGKLKKFFSNKMVYLKDNTLSLKNGKKENIDREYSETDI |
| | | LESDVRDKKNFAVLKKIYLNENVNSEELEVFRNDIKKKLNKINSLKYSFE |
| | | KNKANYQKINENNIEKVEGKSKRNIIYDYYRESAKRDAYVSNVKEAFDK |
| | | LYKEEDIAKLVLEIENLTKLEKYKIREFYHEIIGRKNDKENFAKIIYEEIQN |
| | | VNNMKELIEKVPDMSELKKSQVFYKYYLDKEELNDKNIKYAFCHFVEIE |
| | | MSQLLKNYVYKRLSNISNDKIKRIFEYQNLKKLIENKLLNKLDTYVRNC |
| | | GKYNYYLQDGEIATSDFIARNRQNEAFLRNIIGVSSVAYFSLRNILETENE |
| | | NDITGRMRGKTVKNNKGEEKYVSGEVDKIYNENKKNEVKENLKMFYS |
| | | YDFNMDNKNEIEDFFANIDEAISSIRHGIVHFNLELEGKDIFAFKNIAPSEI |
| | | SKKMFQNEINEKKLKLKIFRQLNSANVFRYLEKYKILNYLKRTRFEFVNK |
| | | NIPFVPSFTKLYNRIDNLKGSNALNLGYINIPKRKEVRDSQIYLLKNIYYG |
| | | EFVEEFVNNNDNFEKIFREIIEINKNAGTNTKTKFYKLEKFETLKANTPTE |
| | | YLEKLQSLHQINYDREKIEEDKDIYVDFVQKIFLKGFINYLQESGSLKYLL |
| | | NLEKNEVIANKKSSYDEKLKLWQNNGSDLSKMPEEIYEYIKKIKINKINY |
| | | SDRMSIFYLLLKLIDYKELTNLRGNLEKYESMNKNKIYSEELNIVNLVSL |
| | | DNNKVRANFELEAEDVRKFLKTETLIKDARQLNNFSERFSDGENVIKHR |
| | | SFYNIKKYGILDLLEKIVAKADLKITKEEIKKYENLQKELEKNDFYKIQEK |
| | | IHKKYNQKPNLILRTENKKDFNDYKKAIENIQNYTQLKNKIEFNDLNLLQ |
| | | SLLFRILHRLAGYTSLWERDLQFKLKGEFPEDKYIDEIFNSDRNNNQKYK |
| | | SGGIAYKYVDFLIEKEEGKRAGKNKVKKRSEKEGSFIIRNYIAHFNYIPDA |
| | | EKSILEMLEELRALLKYDRKLKNAVMKSVKDIFKEYGFIVEFGISHESNS |
| | | KKIKVVNVESEKIKHLKNNGLVTKRNSEDLCNLVKVMLEYKKS |
| | 96 | MKVTKVGGISHKKYTSEGRLVKSESEENRTDERLSALLNMRLDMYIKNP |
| | | SSTETKENQKRIGKLKKFFSNKMVYLKDNTLSLKNGKKENIDREYSETDI |
| | | LESDVRDKKNFAVLKKIYLNENVNSEELEVFRNDIKKKLNKINSLKYSFE |
| | | KNKANYQKINENNIEKVEGKSKRNIIYDYYRESAKRDAYVSNVKEAFDK |
| | | LYKEEDIAKLVLEIENLTKLEKYKIREFYHEIIGRKNDKENFAKIIYEEIQN |
| | | VNNMKELIEKVPDMSELKKSQVFYKYFLENEELNDENIKFVFCYFVEIEV |
| | | SDLLKGNVYKASKIYENKIKNIFEYDKLKKLVINKLENKLNNYVRNCGK |
| | | YNYHMENGDIATSDINMRNRQTEAFLRSMIGVSSFGYFSLRNILGVNDD |
| | | DFYEIEEDLTKDERKKESSTLKKVKEDIASKSIFEKVVDKSFQKKGIYNIK |
| | | ENLKMFYGDSFDDADEDELKQFFVNMLNGITSIRHRVVHYNMNTNSENI |
| | | FNFSDIEVSRLLKNIFEKETDKRELKLKIFKQLNSAGVFDYWESWKIKKY |
| | | LENIEFKFVNKNIPFVPSFTKLYNRIDNLKGSNALNLGYINIPKRKEVRDS |
| | | QIYLLKNIYYGEFVEEFVNNNDNFEKIFREIIEINKNAGTNTKTKFYKLEK |
| | | FETLKANTPTEYLEKLQSLHQINYDREKIEEDKDIYVDFVQKIFLKGFINY |
| | | LQESGSLKYLLNLEKNEVIANKKSSYDEKLKLWQNNGSDLSKMPEEIYE |
| | | YIKKIKINKINYSDRMSIFYLLLKLIDYKELTNLRGNLEKYESMNKNKIYS |
| | | EELNIVNLVSLDNNKVRANFELEAEDVRKFLKTETLIKDARQLNNFSERF |
| | | SDGENVIKHRSFYNIKKYGILDLLEKIVAKADLKITKEEIKKYENLQKELE |
| | | KNDFYKIQEKIHKKYNQKPNLILRTENKKDFNDYKKAIENIQNYTQLKN |
| | | KIEFNDLNLLQSLLFRILHRLAGYTSLWERDLQFKLKGEFPENQYIEEIFN |
| | | FENKKNVKYKGGQIVEKYIKFYKELHQNDEVKINKYSSANIKVLKQEKK |
| | | DLYIRNYIAHFNYIPHAEISLLEVLENLRKLLSYDRKLKNAVMKSVVDIL |
| | | KEYGFVATFKIGADKKIGIQTLESEKIVHLKNLKKKKLMTDRNSEELCKL |
| | | VKIMFEYKMEEKKSEN |
| | 97 | MKVTKIDGISHKKYEEEGKLVKSKNIKEKNKTEERFNDIEKKLKELLEIK |
| | | LDNYIKNPDNAKEEENKIRRENLKEYFSKIILHLEDEKLILKNKKKLKNIV |
| | | QNKNYEEEDINEYDLKNKKSFNVLKKILKLKLNENVKEEKITENSEELKE |
| | | VFGKDLKKKLNQIKSIKNSLEKNKANYKKESIEIGNNNIEKVKGKNKRSII |
| | | YEYYRKSAKHEKYVNNIKEAFDKLYTNSEEDIKKLFLEIEKLTKKTHEKS |
| | | KIREFYHNEIIGKNNFSKDKGDFAKILYEEIQNSINKEENFKELIEKVGNPE |
| | | LKELTKSQIFYKYYLDKEELNDENIKYVFCHFVEIEVSKLLKNYVYKKPS |
| | | NINKDKIKNIFEYKKLKKLIENKLLNKLNTYVRNCGKYNYYLENGDIATS |
| | | DIIMRNRQNEAFLRSIIGVSSFAYFSLRNILGTDNEDFYEIEEDITGKERGK |
| | | ESNVLKTAKENIGEEKIVEGEVDKIYEENKKNEIKENLKMFYGNSFDMD |
| | | NENEIKEFFVNIDEAISSIRHGIVHFNMDTNSENIFNFNNIEVSKISKKIFEN |
| | | EINEKKLKLKIFRQLNSANVFDYLEKWKIKKYLKNIKFKFVNKNIPFVPSF |
| | | TKLYNRIDDLKGSNSLKIGYWKIPKKKEAKDAQIYLLKNIYYGEFLDKFV |
| | | NNKNGNFFKISKEIIEINKNAGKNKKTGFYKLEKFENIEETTPKEYLEIIQS |
| | | LYMINYGKEKDEEEKDIYIDFIQKIFLKGFIDYLKKNNSLKLLELLNLKEK |
| | | NEIIADKKSKYDKKLKKYEKNNRNLIEIPKEINEFIKKIKIAEINKILNYTD |
| | | RLNIFYLLLKLLNHKELTNLKGNLEKYQSMNKNEIYSEELELINLVNLDN |
| | | NKVKIENFELEAEEIGKFLETNSRGNKIKNIKELKNEDTIENTGEYNKIYA |
| | | DGENIIKHRSFYNIKKYGMLDLLEKISEKANYKISKEELKKYSNLKNELE |
| | | DEKNDFYKIQENLHRKYNRKPKNILKKENKEDYENYKKAIENIEEYTHL |
| | | KNKIEFNELNLLQSLLLRILHRLVGYTSIWERDLRFRLKGEFPEDQYIEEIF |
| | | NFDNKKNKKYKSGQIAEKYIKFLKEKEEGKKAKILGYNSKKIKKLKEKE |
| | | GNLFIRNYIAHFNYIPNAEISILEILEKLRELLSYDRKLKNAVMKSVKDILK |
| | | EYGFIVEFKISAESNDKKIKILSVKSEEIVHLKKLKLKDNDKKKNGLKTY |
| | | RNSEDLCKLVKVMFEYKKEN |
| | 98 | MKVTKIDGISHKKYKEEGKLVKSTSIKKEEKSNNDTNKEEKNKTEERFN |
| | | DIEKKLSELLSIKLDIYIKNPDNASNKDKDAEEENKIRRENLKEYFSNIVL |
| | | HLEDGKLYLKNKKELKNIVQNKNYSEEDISEYDLKNKNSFNVLKKILLL |
| | | KLNENVKEEKIKFNSEELKEVFRKDIKKKLNQIKSIKNSLEKNKANYKKE |
| | | SIEIGNNNIEKVKGKNKRSIIYEYYRESAKHEEYVSNIKEAFDKLYTNSEE |
| | | DIAKLFLEIENLTKKTHEKYKIREFYHNEIIGKNCDNFSKDKEDFAKILYE |
| | | EIQNSINKEENFKELIEKVGNPEMKELTKSQIFYKYYLDKEELNDENIKY |
| | | VFCHFVEIEVSKLLKNYVYKKPSFNNINKDKIKNIFEYKKLKKLIENKLL |
| | | NKLNTYVRNCGKYNYYLQNGDIAVTSDFIVGNRQNEAFLRSIIGVSSVA |
| | | YFSLRNILETDNEDFYEIEEDITGKMRGKESNILKEDSILKTVIKNNKGET |
| | | NEDKIVSGEVDKIYNENNKKNEVKENLKMFYGNDFDMDNKNEIKDFFV |
| | | NIDEAISSIRHGIVHFNMDANGKNIFAFNNIELSEISKKIFQNEINEKKLKL |
| | | KIFRQLNSANVFDYLEKYKIIKYLKNTKFNFVNKNIPFVPSFTKLYNRIDD |
| | | LKGNNSLKIGNYWKIPKTNDDNKEKEAKDAQIYLLKNIYYGEFLDKFVN |
| | | NKNGNFFKISKEIIELNKNDKRNKKTGFYKLEKFENIEETIPKEYLEIIQSL |
| | | YMINYGKEKDEEEKDIYIDFIQKIFLKGFIDYLKKNNSLKLLELGNNNDN |
| | | EETFSRNKIIADKKNKFDKKLKKYEKNNRNLIEIPKEINEFVRKIKIAEINK |
| | | ILNYTDRLNMFYLLLKLLNHKELTNLKGNLEKYQSMNKNEIYSEELELIN |
| | | LVNLDNNKVKIENFELEAEEIGKFLETNSIEEINRGNKIKNLKELKNEDTIE |
| | | NTGEYNKIYSDGENIIKHRSFYNIKKYGMLDLLEKISEKANYKISKEELK |
| | | KYSNLKNELEDEKNDFYKIQENLHRKYARKRKDILKKNNDEDYENYKK |
| | | AIENIEEYTHLKNKIEFNELNLLQSLLLRILHRLVGYTSIWERDLRFRLKG |
| | | EFPEDQYIEEIFNFDNKKNVKYNEKNEKSGQIVEKYIKFLKELHENEEAKI |
| | | NKYNSAKIKKLKEIIEEKNLYIRNYIAHFNYIPNAEISILEILENLRELLSYD |
| | | RKLKNAVMKSVKDILKEYGFVVEFKIGAESNDKKIEIQSVKSEEIVHLKK |
| | | LKLKDNDKKKNPLKTYRNSEDLCKLVKVMFEYKMEEKKSEN |
| | 99 | MKVTKIDGISHKKYKEEGKLVKSESEENRTDERLSALLNMRLDMYIKNP |
| | | SSTETKENQKRIGKLKKFFSNIVLHLEDGKLYLKNKKELKNIVQNKNYSE |
| | | EDISEYDLKNKNSFNVLKKILLLKLNENVKEEKIKFNSEELKEVFRKDIK |
| | | KKLNQIKSIKNSLEKNKANYKKESIEIGNNNIEKVKGKNKRSIIYEYYRES |
| | | AKHEEYVSNIKEAFDKLYTNSEEDIAKLFLEIENLTKKTHEKYKIREFYH |
| | | NEIIGRKNDKEDFAKILYEEIQNSINKEENFKELIEKVGNPEMKELTKSQIF |
| | | YKYYLDKEELNDENIKYVFCHFVEIEVSKLLKNYVYKKPSFNNINKDKIK |
| | | NIFEYKKLKKLIENKLLNKLNTYVRNCGKYNYYLQNGDIAVTSDFIVGN |
| | | RQNEAFLRSIIGVSSVAYFSLRNILETDNENDITGRMRGKTVKNNKGEEK |
| | | YVSGEVDKIYNENNKKNEVKENLKMFYGNDFDMDNKNEIKDFFVNIDE |
| | | AISSIRHGIVHFNMDANGKNIFAFNNIELSEISKKIFQNEINEKKLKLKIFR |
| | | QLNSANVFDYLEKYKIIKYLKNTKFNFVNKNIPFVPSFTKLYNRIDDLKG |
| | | NNSLKIGNYWKIPKTNDDNKEKEAKDAQIYLLKNIYYGEFLDKFVNNKN |
| | | GNFFKISKEIIELNKNDKRNKKTGFYKLEKFENIEETIPKEYLEIIQSLYMI |
| | | NYGKEKDEEEKDIYIDFIQKIFLKGFIDYLNKNNLKYIESNNNNDNNDIFS |
| | | KIKIKKDNKEKYDKILKKYEKNNRNLIEIPKEINEFVRKIKIAEINKILNYT |
| | | DRLNMFYLLLKLLNHKELTNLKGNLEKYQSMNKNEIYSEELELINLVNL |
| | | DNNKVKIENFELEAEEIGKFLDFNENKIKDRKELKKFDTNKIYSDGENIIK |
| | | HRSFYNIKKYGMLDLLEKISEKANYKISKEELKKYSNLKNELEDEKNDF |
| | | YKIQENLHRKYARKRKDILKKNNDEDYENYKKAIENIEEYTHLKNKIEF |
| | | NELNLLQSLLLRILHRLVGYTSIWERDLRFRLKGEFPEDQYIEEIFNFDNK |
| | | KNVKYNEKNEKSGQIVEKYIKFLKELHENEEAKINKYNSAKIKKLKEIIE |
| | | EKNLYIRNYIAHFNYIPNAEISILEILENLRELLSYDRKLKNAVMKSVKDI |
| | | LKEYGFVVEFKIGAESNDKKIEIQSVKSEEIVHLKNLKKKKLMTDRNSED |
| | | LCKLVKVMFEYKMEEKKSEN |

TABLE 1.1 provides exemplary amino acid alterations relative to SEQ ID NO: 69 useful in compositions, systems, and methods described herein.

TABLE 1.1
Exemplary Amino Acid Alterations relative to Effector Protein(s)

| | |
|---|---|
| Relative to | |
| SEQ ID NO: | Exemplary Amino Acid Alterations |

Exemplary Amino Acid Alterations

| | |
|---|---|
| 69 | T9G; T15K; Q56A; H106Q; E121N; C125L; E131K; H139Q; N150Y; G154K; H164R; |
| | Q166K; Q175K; D184E; E198K; F200Y; A242M; R247T; A262T; N265R; N281K; |
| | D289T; H305K; M311V; A313S; N314K; K318Q; E333H; L338I; S352C; V372L; |
| | L381M; Q480Y; Q485S; V492Q; H496S; V501T; G517N; S521A; L537R; E543L; |
| | W546Y; S547N; G548A; I555L; Y559N; N567S; D569H; D574Q; L579V; Q585L; |
| | Q618E; W621Q; I622N; I622K; M623L; D631C; L647V; M656L; L684K; A705T; |
| | Q709H; K717M; N722R; T726Q; A737S; T747L; A762G; W765R; W765N; Q766M; |
| | K769E; T789Q; N790K; D800K; D800G; D800R; E801M; E801G; S807T; S807R; |
| | S819K; S827R; S827K; N836P; N843S; A846P; T847K; E857M; Y858L; E864Q; E867A; |
| | D870E; N871K; N909D; N909E; E915V; S919Q; S919K; I923L; M927I; D974Y; |
| | L1011V; H1020R; A1030S; T1032V; D1035M; D1049G; Q1054T; Q1056L; S1062P; |
| | I1064K; P1083Q; A1085E* |

Exemplary Combinations of 2 Alterations

| | |
|---|---|
| 69 | E121N, H139Q; E121N, M311V; E121N, D184E; E121N, G154K; E121N, S547N; |
| | E121N, K318Q; E121N, M656L; E121N, V372L; E121N, Y858L; E121N, G548A; |
| | E121N, S352C; E121N, M927I; E121N, A737S; E121N, S1062P; E121N, S819K; E121N, |
| | V501T; E121N, D974Y; E121N, I1064K; E121N, N722R; E121N, W621Q; E121N, |
| | W765N; E121N, I622N; E121N, S807R; E121N, A762G; E121N, N871K; E121N, |
| | D800R; E121N, S827K; E121N, H1020R; H139Q, M311V; H139Q, D184E; H139Q, |
| | G154K; H139Q, S547N; H139Q, K318Q; H139Q, M656L; H139Q, V372L; H139Q, |
| | Y858L; H139Q, G548A; H139Q, S352C; H139Q, M927I; H139Q, A737S; H139Q, |
| | S1062P; H139Q, S819K; H139Q, V501T; H139Q, D974Y; H139Q, I1064K; H139Q, |
| | N722R; H139Q, W621Q; H139Q, W765N; H139Q, I622N; H139Q, S807R; H139Q, |
| | A762G; H139Q, N871K; H139Q, D800R; H139Q, S827K; H139Q, H1020R; M311V, |
| | D184E; M311V, G154K; M311V, S547N; M311V, K318Q; M311V, M656L; M311V, |
| | V372L; M311V, Y858L; M311V, G548A; M311V, S352C; M311V, M927I; M311V, |
| | A737S; M311V, S1062P; M311V, S819K; M311V, V501T; M311V, D974Y; M311V, |
| | I1064K; M311V, N722R; M311V, W621Q; M311V, W765N; M311V, I622N; M311V, |
| | S807R; M311V, A762G; M311V, N871K; M311V, D800R; M311V, S827K; M311V, |
| | H1020R; D184E, G154K; D184E, S547N; D184E, K318Q; D184E, M656L; D184E, |
| | V372L; D184E, Y858L; D184E, G548A; D184E, S352C; D184E, M927I; D184E, A737S; |
| | D184E, S1062P; D184E, S819K; D184E, V501T; D184E, D974Y; D184E, I1064K; |
| | D184E, N722R; D184E, W621Q; D184E, W765N; D184E, I622N; D184E, S807R; |
| | D184E, A762G; D184E, N871K; D184E, D800R; D184E, S827K; D184E, H1020R; |
| | G154K, S547N; G154K, K318Q; G154K, M656L; G154K, V372L; G154K, Y858L; |
| | G154K, G548A; G154K, S352C; G154K, M927I; G154K, A737S; G154K, S1062P; |
| | G154K, S819K; G154K, V501T; G154K, D974Y; G154K, I1064K; G154K, N722R; |
| | G154K, W621Q; G154K, W765N; G154K, I622N; G154K, S807R; G154K, A762G; |
| | G154K, N871K; G154K, D800R; G154K, S827K; G154K, H1020R; S547N, K318Q; |
| | S547N, M656L; S547N, V372L; S547N, Y858L; S547N, G548A; S547N, S352C; S547N, |
| | M927I; S547N, A737S; S547N, S1062P; S547N, S819K; S547N, V501T; S547N, D974Y; |
| | S547N, I1064K; S547N, N722R; S547N, W621Q; S547N, W765N; S547N, I622N; |
| | S547N, S807R; S547N, A762G; S547N, N871K; S547N, D800R; S547N, S827K; S547N, |
| | H1020R; K318Q, M656L; K318Q, V372L; K318Q, Y858L; K318Q, G548A; K318Q, |
| | S352C; K318Q, M927I; K318Q, A737S; K318Q, S1062P; K318Q, S819K; K318Q, |
| | V501T; K318Q, D974Y; K318Q, I1064K; K318Q, N722R; K318Q, W621Q; K318Q, |
| | W765N; K318Q, I622N; K318Q, S807R; K318Q, A762G; K318Q, N871K; K318Q, |
| | D800R; K318Q, S827K; K318Q, H1020R; M656L, V372L; M656L, Y858L; M656L, |
| | G548A; M656L, S352C; M656L, M927I; M656L, A737S; M656L, S1062P; M656L, |
| | S819K; M656L, V501T; M656L, D974Y; M656L, I1064K; M656L, N722R; M656L, |
| | W621Q; M656L, W765N; M656L, I622N; M656L, S807R; M656L, A762G; M656L, |
| | N871K; M656L, D800R; M656L, S827K; M656L, H1020R; V372L, Y858L; V372L, |
| | G548A; V372L, S352C; V372L, M927I; V372L, A737S; V372L, S1062P; V372L, S819K; |
| | V372L, V501T; V372L, D974Y; V372L, I1064K; V372L, N722R; V372L, W621Q; |
| | V372L, W765N; V372L, I622N; V372L, S807R; V372L, A762G; V372L, N871K; V372L, |
| | D800R; V372L, S827K; V372L, H1020R; Y858L, G548A; Y858L, S352C; Y858L, |
| | M927I; Y858L, A737S; Y858L, S1062P; Y858L, S819K; Y858L, V501T; Y858L, |
| | D974Y; Y858L, I1064K; Y858L, N722R; Y858L, W621Q; Y858L, W765N; Y858L, |
| | I622N; Y858L, S807R; Y858L, A762G; Y858L, N871K; Y858L, D800R; Y858L, S827K; |
| | Y858L, H1020R; G548A, S352C; G548A, M927I; G548A, A737S; G548A, S1062P; |
| | G548A, S819K; G548A, V501T; G548A, D974Y; G548A, I1064K; G548A, N722R; |
| | G548A, W621Q; G548A, W765N; G548A, I622N; G548A, S807R; G548A, A762G; |
| | G548A, N871K; G548A, D800R; G548A, S827K; G548A, H1020R; S352C, M927I; |
| | S352C, A737S; S352C, S1062P; S352C, S819K; S352C, V501T; S352C, D974Y; S352C, |
| | I1064K; S352C, N722R; S352C, W621Q; S352C, W765N; S352C, I622N; S352C, S807R; |
| | S352C, A762G; S352C, N871K; S352C, D800R; S352C, S827K; S352C, H1020R; M927I, |
| | A737S; M927I, S1062P; M927I, S819K; M927I, V501T; M927I, D974Y; M927I, I1064K; |
| | M927I, N722R; M927I, W621Q; M927I, W765N; M927I, I622N; M927I, S807R; M927I, |
| | A762G; M927I, N871K; M927I, D800R; M927I, S827K; M927I, H1020R; A737S, |
| | S1062P; A737S, S819K; A737S, V501T; A737S, D974Y; A737S, I1064K; A737S, |
| | N722R; A737S, W621Q; A737S, W765N; A737S, I622N; A737S, S807R; A737S, |
| | A762G; A737S, N871K; A737S, D800R; A737S, S827K; A737S, H1020R; S1062P, |
| | S819K; S1062P, V501T; S1062P, D974Y; S1062P, I1064K; S1062P, N722R; S1062P, |
| | W621Q; S1062P, W765N; S1062P, I622N; S1062P, S807R; S1062P, A762G; S1062P, |
| | N871K; S1062P, D800R; S1062P, S827K; S1062P, H1020R; S819K, V501T; S819K, |
| | D974Y; S819K, I1064K; S819K, N722R; S819K, W621Q; S819K, W765N; S819K, |
| | I622N; S819K, S807R; S819K, A762G; S819K, N871K; S819K, D800R; S819K, S827K; |
| | S819K, H1020R; V501T, D974Y; V501T, I1064K; V501T, N722R; V501T, W621Q; |
| | V501T, W765N; V501T, I622N; V501T, S807R; V501T, A762G; V501T, N871K; V501T, |
| | D800R; V501T, S827K; V501T, H1020R; D974Y, I1064K; D974Y, N722R; D974Y, |
| | W621Q; D974Y, W765N; D974Y, I622N; D974Y, S807R; D974Y, A762G; D974Y, |
| | N871K; D974Y, D800R; D974Y, S827K; D974Y, H1020R; I1064K, N722R; I1064K, |
| | W621Q; I1064K, W765N; I1064K, I622N; I1064K, S807R; I1064K, A762G; I1064K, |
| | N871K; I1064K, D800R; I1064K, S827K; I1064K, H1020R; N722R, W621Q; N722R, |
| | W765N; N722R, I622N; N722R, S807R; N722R, A762G; N722R, N871K; N722R, |
| | D800R; N722R, S827K; N722R, H1020R; W621Q, W765N; W621Q, I622N; W621Q, |
| | S807R; W621Q, A762G; W621Q, N871K; W621Q, D800R; W621Q, S827K; W621Q, |
| | H1020R; W765N, I622N; W765N, S807R; W765N, A762G; W765N, N871K; W765N, |
| | D800R; W765N, S827K; W765N, H1020R; I622N, S807R; I622N, A762G; I622N, |
| | N871K; I622N, D800R; I622N, S827K; I622N, H1020R; S807R, A762G; S807R, N871K; |
| | S807R, D800R; S807R, S827K; S807R, H1020R; A762G, N871K; A762G, D800R; |
| | A762G, S827K; A762G, H1020R; N871K, D800R; N871K, S827K; N871K, H1020R; |
| | D800R, S827K; D800R, H1020R; S827K, H1020R* |

Exemplary Combinations of 3 Amino Acid Alterations

| | |
|---|---|
| 69 | E121N, M311V, S547N; H139Q, D184E, K318Q; H139Q, D184E, M311V; H139Q, |
| | G154K, D184E; H106Q, E121N, C125L; E121N, M311V, Q618E; E121N, Y858L, |
| | S1062P; H106Q, E121N, M311V; H106Q, F200Y, M311V; F200Y, S547N, L579V; |
| | H106Q, F200Y, S547N; F200Y, M311V, W546Y; H139Q, K318Q, S352C; H139Q, |
| | H164R, K318Q; H139Q, V372L, G548A; H139Q, Q175K, D184E; H139Q, L338I, |
| | V372L; H139Q, K318Q, L338I; H139Q, D184E, G517N; Q175K, D184E, S352C; H139Q, |
| | V372L, L381M; H139Q, Q175K, V372L; H139Q, D184E, R247T; T9G, H139Q, D184E; |
| | H139Q, D184E, A313S; H139Q, N150Y, D184E; H139Q, Q166K, D184E; H139Q, |
| | D184E, A242M; Q56A, H139Q, D184E; H139Q, D184E, N281K; H139Q, D184E, |
| | A262T; H139Q, D184E, D289T; H139Q, D184E, N265R; T15K, H106Q, E121N; E131K, |
| | H139Q, D184E; H139Q, D184E, H305K; H106Q, H139Q, D184E; T15K, H106Q, E131K; |
| | T15K, H106Q, H139Q; H139Q, D184E, F200Y; H139Q, D184E, E198K; H106Q, E131K, |
| | H139Q; T15K, E121N, E131K |

Exemplary Combinations of 4 Amino Acid Alterations

| | |
|---|---|
| 69 | E121N, M311V, S547N, M656L; H139Q, D184E, K318Q, V372L; H139Q, D184E, |
| | M311V, K318Q; H139Q, G154K, D184E, M311V; H106Q, E121N, C125L, M311V; |
| | E121N, M311V, Q618E, M656L; H106Q, E121N, M311V, Q480Y; H106Q, F200Y, |
| | M311V, Q480Y; F200Y, S547N, L579V, M656L; H106Q, F200Y, S547N, M656L; |
| | F200Y, M311V, W546Y, Y559N; H139Q, K318Q, S352C, G548A; H139Q, H164R, |
| | K318Q, S352C; H139Q, V372L, G548A, T747L; H139Q, Q175K, D184E, K318Q; |
| | H139Q, L338I, V372L, G517N; H139Q, K318Q, L338I, V372L; H139Q, D184E, G517N, |
| | G548A; Q175K, D184E, S352C, V372L; H139Q, V372L, L381M, G517N; H139Q, |
| | Q175K, V372L, N909D; H139Q, D184E, R247T, K318Q; T9G, H139Q, D184E, K318Q; |
| | H139Q, D184E, A313S, K318Q; H139Q, N150Y, D184E, K318Q; H139Q, Q166K, |
| | D184E, K318Q; H139Q, D184E, K318Q, E333H; H139Q, G154K, D184E, K318Q; |
| | H139Q, D184E, A242M, K318Q; Q56A, H139Q, D184E, K318Q; H139Q, D184E, |
| | N281K, K318Q; H139Q, D184E, A262T, K318Q; H139Q, D184E, D289T, K318Q; |
| | H139Q, D184E, N265R, K318Q; H139Q, D184E, K318Q, S352C; T15K, H106Q, E121N, |
| | E131K; E131K, H139Q, D184E, E198K; H139Q, D184E, H305K, M311V; H106Q, |
| | H139Q, D184E, F200Y; T15K, H106Q, E131K, H139Q; H106Q, H139Q, D184E, |
| | M311V; T15K, H106Q, H139Q, D184E; H139Q, D184E, F200Y, K318Q; H139Q, |
| | D184E, E198K, H305K; H106Q, E131K, H139Q, D184E; T15K, E121N, E131K, H139Q* |

Exemplary Combinations of 5 Amino Acid Alterations

| | |
|---|---|
| 69 | E121N, M311V, S547N, M656L, Y858L; H139Q, D184E, K318Q, V372L, G548A; |
| | H139Q, D184E, M311V, K318Q, S352C; H139Q, G154K, D184E, M311V, K318Q; |
| | H106Q, E121N, C125L, M311V, S547N; E121N, M311V, Q618E, M656L, Y858L; |
| | E121N, M311V, S547N, M656L, M927I; H106Q, E121N, M311V, Q480Y, S521A; |
| | H106Q, F200Y, M311V, Q480Y, S547N; F200Y, S547N, L579V, M656L, Y858L; |
| | H106Q, F200Y, S547N, M656L, S1062P; F200Y, M311V, W546Y, Y559N, M656L; |
| | H139Q, K318Q, S352C, G548A, N909D; H139Q, H164R, K318Q, S352C, V372L; |
| | H139Q, V372L, G548A, T747L, I1064K; H139Q, Q175K, D184E, K318Q, I555L; |
| | H139Q, Q175K, D184E, K318Q, L338I; H139Q, L338I, V372L, G517N, A737S; H139Q, |
| | K318Q, L338I, V372L, G548A; H139Q, D184E, G517N, G548A, I1064K; Q175K, |
| | D184E, S352C, V372L, H496S; H139Q, V372L, L381M, G517N, A737S; H139Q, |
| | Q175K, V372L, N909D, I1064K; H139Q, D184E, K318Q, V372L, V501T; H139Q, |
| | D184E, K318Q, V372L, V492Q; H139Q, D184E, K318Q, V372L, L537R; H139Q, |
| | D184E, K318Q, V372L, Q485S; H139Q, D184E, R247T, K318Q, V372L; T9G, H139Q, |
| | D184E, K318Q, V372L; H139Q, D184E, A313S, K318Q, V372L; H139Q, N150Y, |
| | D184E, K318Q, V372L; H139Q, Q166K, D184E, K318Q, V372L; H139Q, D184E, |
| | K318Q, E333H, V372L; H139Q, G154K, D184E, K318Q, V372L; H139Q, D184E, |
| | A242M, K318Q, V372L; Q56A, H139Q, D184E, K318Q, V372L; H139Q, D184E, |
| | N281K, K318Q, V372L; H139Q, D184E, A262T, K318Q, V372L; H139Q, D184E, |
| | D289T, K318Q, V372L; H139Q, D184E, K318Q, V372L, E543L; H139Q, D184E, |
| | N265R, K318Q, V372L; H139Q, D184E, K318Q, S352C, V372L; T15K, H106Q, E121N, |
| | E131K, H139Q; E131K, H139Q, D184E, E198K, H305K; H139Q, D184E, H305K, |
| | M311V, K318Q; H106Q, H139Q, D184E, F200Y, K318Q; T15K, H106Q, E131K, |
| | H139Q, D184E; H106Q, H139Q, D184E, M311V, K318Q; T15K, H106Q, H139Q, |
| | D184E, H305K; H139Q, D184E, F200Y, K318Q, S352C; H139Q, D184E, E198K, |
| | H305K, K318Q; H106Q, E131K, H139Q, D184E, F200Y; T15K, E121N, E131K, H139Q, |
| | D184E; H139Q, D184E, F200Y, K318Q, V372L; H106Q, H139Q, D184E, F200Y, |
| | M311V* |

Exemplary Combinations of 6 Amino Acid Alterations

| | |
|---|---|
| 69 | E121N, M311V, S547N, M656L, Y858L, M927I; H139Q, D184E, K318Q, V372L, |
| | G548A, A737S; H139Q, D184E, M311V, K318Q, S352C, V372L; H139Q, G154K, |
| | D184E, M311V, K318Q, S352C; H106Q, E121N, C125L, M311V, S547N, Y559N; |
| | E121N, M311V, S547N, M656L, M927I, S1062P; H106Q, E121N, M311V, Q480Y, |
| | S521A, Q709H; H106Q, F200Y, M311V, Q480Y, S547N, Y559N; F200Y, S547N, |
| | L579V, M656L, Y858L, S1062P; F200Y, M311V, W546Y, Y559N, M656L, Y858L; |
| | H139Q, K318Q, S352C, G548A, N909D, I1064K; H139Q, H164R, K318Q, S352C, |
| | V372L, I1064K; H139Q, V372L, G548A, T747L, I1064K, ; H139Q, Q175K, D184E, |
| | K318Q, I555L, I1064K; H139Q, Q175K, D184E, K318Q, L338I, V372L; H139Q, L338I, |
| | V372L, G517N, A737S, N909D; H139Q, K318Q, L338I, V372L, G548A, T747L; Q175K, |
| | D184E, S352C, V372L, H496S, G517N; H139Q, V372L, L381M, G517N, A737S, |
| | I1064K; H139Q, D184E, K318Q, V372L, G548A, D569H; H139Q, D184E, K318Q, |
| | V372L, G548A, Q585L; H139Q, D184E, K318Q, V372L, G548A, N722R; H139Q, |
| | D184E, K318Q, V372L, G548A, N567S; H139Q, D184E, K318Q, V372L, V501T, |
| | G548A; H139Q, D184E, K318Q, V372L, V492Q, G548A; H139Q, D184E, K318Q, |
| | V372L, L537R, G548A; H139Q, D184E, K318Q, V372L, Q485S, G548A; H139Q, |
| | D184E, K318Q, V372L, G548A, D574Q; H139Q, D184E, R247T, K318Q, V372L, |
| | G548A; T9G, H139Q, D184E, K318Q, V372L, G548A; H139Q, D184E, A313S, K318Q, |
| | V372L, G548A; H139Q, N150Y, D184E, K318Q, V372L, G548A; H139Q, D184E, |
| | K318Q, V372L, G548A, I622N; H139Q, D184E, K318Q, V372L, G548A, M623L; |
| | H139Q, Q166K, D184E, K318Q, V372L, G548A; H139Q, D184E, K318Q, V372L, |
| | G548A, T726Q; H139Q, D184E, K318Q, E333H, V372L, G548A; H139Q, G154K, |
| | D184E, K318Q, V372L, G548A; H139Q, D184E, A242M, K318Q, V372L, G548A; |
| | Q56A, H139Q, D184E, K318Q, V372L, G548A; H139Q, D184E, N281K, K318Q, V372L, |
| | G548A; H139Q, D184E, K318Q, V372L, G548A, W621Q; H139Q, D184E, A262T, |
| | K318Q, V372L, G548A; H139Q, D184E, K318Q, V372L, G548A, D631C; H139Q, |
| | D184E, D289T, K318Q, V372L, G548A; H139Q, D184E, K318Q, V372L, E543L, |
| | G548A; H139Q, D184E, K318Q, V372L, G548A, I622K; H139Q, D184E, K318Q, |
| | V372L, G548A, L684K; H139Q, D184E, N265R, K318Q, V372L, G548A; H139Q, |
| | D184E, K318Q, S352C, V372L, G548A; T15K, H106Q, E121N, E131K, H139Q, D184E; |
| | E131K, H139Q, D184E, E198K, H305K, M311V; H139Q, D184E, H305K, M311V, |
| | K318Q, S352C; H106Q, H139Q, D184E, F200Y, K318Q, S352C; T15K, H106Q, E131K, |
| | H139Q, D184E, M311V; H106Q, H139Q, D184E, M311V, K318Q, S352C; T15K, |
| | H106Q, H139Q, D184E, H305K, M311V; H139Q, D184E, F200Y, K318Q, S352C, |
| | V372L; H139Q, D184E, E198K, H305K, K318Q, S352C; H106Q, E131K, H139Q, |
| | D184E, F200Y, M311V; T15K, E121N, E131K, H139Q, D184E, F200Y; H106Q, H139Q, |
| | D184E, M311V, K318Q, V372L; H139Q, D184E, F200Y, K318Q, V372L, G548A; |
| | H139Q, D184E, K318Q, V372L, V501T, L537R; H106Q, H139Q, D184E, F200Y, |
| | M311V, K318Q* |

Exemplary Combinations of 7 Amino Acid Alterations

E121N, M311V, S547N, M656L, Y858L, M927I, S1062P; H139Q, D184E, K318Q,
V372L, G548A, A737S, S819K; H139Q, D184E, M311V, K318Q, S352C, V372L,
V501T; H139Q, G154K, D184E, M311V, K318Q, S352C, V372L; H106Q, E121N,
C125L, M311V, S547N, Y559N, Y858L; H106Q, E121N, M311V, Q480Y, S521A,
Q709H, Y858L; H106Q, F200Y, M311V, Q480Y, S547N, Y559N, Y858L; F200Y,
M311V, W546Y, Y559N, M656L, Y858L, S1062P; H139Q, Q175K, D184E, K318Q,
I555L, I1064K, A1085E; H139Q, Q175K, D184E, K318Q, L338I, V372L, G548A;
H139Q, L338I, V372L, G517N, A737S, N909D, I1064K; H139Q, K318Q, L338I, V372L,
G548A, T747L, I1064K; H139Q, D184E, K318Q, V372L, G548A, A737S, S827R;
H139Q, D184E, K318Q, V372L, G548A, D569H, A737S; H139Q, D184E, K318Q,
V372L, G548A, Q585L, A737S; H139Q, D184E, K318Q, V372L, G548A, A737S,
A846P; H139Q, D184E, K318Q, V372L, G548A, A737S, T789Q; H139Q, D184E,
K318Q, V372L, G548A, N722R, A737S; H139Q, D184E, K318Q, V372L, G548A,
N567S, A737S; H139Q, D184E, K318Q, V372L, G548A, A737S, D800K; H139Q,
D184E, K318Q, V372L, G548A, A737S, S919Q; H139Q, D184E, K318Q, V372L,
G548A, A737S, N790K; H139Q, D184E, K318Q, V372L, V501T, G548A, A737S;
H139Q, D184E, K318Q, V372L, G548A, A737S, E801M; H139Q, D184E, K318Q,
V372L, G548A, A737S, T847K; H139Q, D184E, K318Q, V372L, G548A, A737S,
D870E; H139Q, D184E, K318Q, V372L, G548A, A737S, W765R; H139Q, D184E,
K318Q, V372L, G548A, A737S, D800G; H139Q, D184E, K318Q, V372L, G548A,
A737S, W765N; H139Q, D184E, K318Q, V372L, G548A, A737S, E864Q; H139Q,
D184E, K318Q, V372L, V492Q, G548A, A737S; H139Q, D184E, K318Q, V372L,
G548A, A737S, N836P; H139Q, D184E, K318Q, V372L, G548A, A737S, S807T; H139Q,
D184E, K318Q, V372L, L537R, G548A, A737S; H139Q, D184E, K318Q, V372L,
G548A, A737S, S807R; H139Q, D184E, K318Q, V372L, G548A, A737S, E867A;
H139Q, D184E, K318Q, V372L, G548A, A737S, E801G; H139Q, D184E, K318Q,
V372L, G548A, A737S, Q766M; H139Q, D184E, K318Q, V372L, Q485S, G548A,
A737S; H139Q, D184E, K318Q, V372L, G548A, A737S, K769E; H139Q, D184E,
K318Q, V372L, G548A, A737S, N843S; H139Q, D184E, K318Q, V372L, G548A,
A737S, S919K; H139Q, D184E, K318Q, V372L, G548A, D574Q, A737S; H139Q,
D184E, K318Q, V372L, G548A, A737S, E857M; H139Q, D184E, R247T, K318Q,
V372L, G548A, A737S; T9G, H139Q, D184E, K318Q, V372L, G548A, A737S; H139Q,
D184E, A313S, K318Q, V372L, G548A, A737S; H139Q, N150Y, D184E, K318Q,
V372L, G548A, A737S; H139Q, D184E, K318Q, V372L, G548A, I622N, A737S; H139Q,
D184E, K318Q, V372L, G548A, M623L, A737S; H139Q, Q166K, D184E, K318Q,
V372L, G548A, A737S; H139Q, D184E, K318Q, V372L, G548A, T726Q, A737S;
H139Q, D184E, K318Q, E333H, V372L, G548A, A737S; H139Q, G154K, D184E,
K318Q, V372L, G548A, A737S; H139Q, D184E, K318Q, V372L, G548A, A737S,
A762G; H139Q, D184E, A242M, K318Q, V372L, G548A, A737S; Q56A, H139Q,
D184E, K318Q, V372L, G548A, A737S; H139Q, D184E, N281K, K318Q, V372L,
G548A, A737S; H139Q, D184E, K318Q, V372L, G548A, W621Q, A737S; H139Q,
D184E, A262T, K318Q, V372L, G548A, A737S; H139Q, D184E, K318Q, V372L,
G548A, D631C, A737S; H139Q, D184E, D289T, K318Q, V372L, G548A, A737S;
H139Q, D184E, K318Q, V372L, E543L, G548A, A737S; H139Q, D184E, K318Q,
V372L, G548A, I622K, A737S; H139Q, D184E, K318Q, V372L, G548A, L684K, A737S;
H139Q, D184E, N265R, K318Q, V372L, G548A, A737S; H139Q, D184E, K318Q,
S352C, V372L, G548A, A737S; T15K, H106Q, E121N, E131K, H139Q, D184E, H305K;
E131K, H139Q, D184E, E198K, H305K, M311V, K318Q; H139Q, D184E, H305K,
M311V, K318Q, S352C, V372L; H106Q, H139Q, D184E, F200Y, K318Q, S352C,
V372L; T15K, H106Q, E131K, H139Q, D184E, M311V, K318Q; H106Q, H139Q,
D184E, M311V, K318Q, S352C, V372L; T15K, H106Q, H139Q, D184E, H305K,
M311V, K318Q; H139Q, D184E, F200Y, K318Q, S352C, V372L, G548A; H139Q,
D184E, E198K, H305K, K318Q, S352C, V372L; H106Q, E131K, H139Q, D184E, F200Y,
M311V, K318Q; T15K, E121N, E131K, H139Q, D184E, F200Y, K318Q; H106Q,
H139Q, D184E, M311V, K318Q, V372L, G548A; H139Q, D184E, F200Y, K318Q,
V372L, G548A, K717M; H139Q, D184E, K318Q, V372L, V501T, L537R, G548A;
H139Q, D184E, F200Y, K318Q, V372L, G548A, D569H; H106Q, H139Q, D184E,
F200Y, M311V, K318Q, V372L; H139Q, D184E, K318Q, V372L, V501T, G548A,
D569H; H139Q, D184E, M311V, K318Q, S352C, V372L, V492Q; H139Q, D184E,
F200Y, K318Q, V372L, G548A, A737S; H139Q, D184E, K318Q, V372L, G548A,
N567S, N722R*

Exemplary Combinations of 8 Amino Acid Alterations

H139Q, D184E, K318Q, V372L, G548A, A737S, S819K, D974Y; H139Q, D184E,
M311V, K318Q, S352C, V372L, V501T, G548A; H139Q, G154K, D184E, M311V,
K318Q, S352C, V372L, V501T; H106Q, E121N, C125L, M311V, S547N, Y559N,
Y858L, E915V; H106Q, E121N, C125L, M311V, S547N, Y559N, Y858L, S1062P;
H106Q, E121N, M311V, Q480Y, S521A, Q709H, Y858L, E915V; H106Q, F200Y,
M311V, Q480Y, S547N, Y559N, Y858L, M927I; H139Q, Q175K, D184E, K318Q, L338I,
V372L, G548A, T747L; H139Q, K318Q, L338I, V372L, G548A, T747L, I1064K,
A1085E; H139Q, D184E, K318Q, V372L, G548A, A737S, S827R, I1064K; H139Q,
D184E, K318Q, V372L, G548A, D569H, A737S, I1064K; H139Q, D184E, K318Q,
V372L, G548A, Q585L, A737S, I1064K; H139Q, D184E, K318Q, V372L, G548A,
A737S, A846P, I1064K; H139Q, D184E, K318Q, V372L, G548A, A737S, T789Q,
I1064K; H139Q, D184E, K318Q, V372L, G548A, N722R, A737S, I1064K; H139Q,
D184E, K318Q, V372L, G548A, N567S, A737S, I1064K; H139Q, D184E, K318Q,
V372L, G548A, A737S, D800K, I1064K; H139Q, D184E, K318Q, V372L, G548A,
A737S, S919Q, I1064K; H139Q, D184E, K318Q, V372L, G548A, A737S, N790K,
I1064K; H139Q, D184E, K318Q, V372L, V501T, G548A, A737S, I1064K; H139Q,
D184E, K318Q, V372L, G548A, A737S, E801M, I1064K; H139Q, D184E, K318Q,
V372L, G548A, A737S, T847K, I1064K; H139Q, D184E, K318Q, V372L, G548A,
A737S, D870E, I1064K; H139Q, D184E, K318Q, V372L, G548A, A737S, W765R,
I1064K; H139Q, D184E, K318Q, V372L, G548A, A737S, D800G, I1064K; H139Q,
D184E, K318Q, V372L, G548A, A737S, W765N, I1064K; H139Q, D184E, K318Q,
V372L, G548A, A737S, E864Q, I1064K; H139Q, D184E, K318Q, V372L, V492Q,
G548A, A737S, I1064K; H139Q, D184E, K318Q, V372L, G548A, A737S, N836P,
I1064K; H139Q, D184E, K318Q, V372L, G548A, A737S, S807T, I1064K; H139Q,
D184E, K318Q, V372L, L537R, G548A, A737S, I1064K; H139Q, D184E, K318Q,
V372L, G548A, A737S, S807R, N871K; H139Q, D184E, K318Q, V372L, G548A,
A737S, E867A, I1064K; H139Q, D184E, K318Q, V372L, G548A, A737S, E801G,
I1064K; H139Q, D184E, K318Q, V372L, G548A, A737S, Q766M, I1064K; H139Q,
D184E, K318Q, V372L, Q485S, G548A, A737S, I1064K; H139Q, D184E, K318Q,
V372L, G548A, A737S, K769E, I1064K; H139Q, D184E, K318Q, V372L, G548A,
A737S, N843S, I1064K; H139Q, D184E, K318Q, V372L, G548A, A737S, S919K,
I1064K; H139Q, D184E, K318Q, V372L, G548A, D574Q, A737S, I1064K; H139Q,
D184E, K318Q, V372L, G548A, A737S, E857M, I1064K; H139Q, D184E, R247T,
K318Q, V372L, G548A, A737S, S819K; T9G, H139Q, D184E, K318Q, V372L, G548A,
A737S, S819K; H139Q, D184E, A313S, K318Q, V372L, G548A, A737S, S819K; H139Q,
N150Y, D184E, K318Q, V372L, G548A, A737S, S819K; H139Q, D184E, K318Q,
V372L, G548A, I622N, A737S, S819K; H139Q, D184E, K318Q, V372L, G548A, M623L,
A737S, S819K; H139Q, Q166K, D184E, K318Q, V372L, G548A, A737S, S819K;
H139Q, D184E, K318Q, V372L, G548A, T726Q, A737S, S819K; H139Q, D184E,
K318Q, E333H, V372L, G548A, A737S, S819K; H139Q, G154K, D184E, K318Q,
V372L, G548A, A737S, S819K; H139Q, D184E, K318Q, V372L, G548A, A737S,
A762G, S819K; H139Q, D184E, A242M, K318Q, V372L, G548A, A737S, S819K;
H139Q, D184E, K318Q, V372L, G548A, A737S, S819K, N909E; Q56A, H139Q, D184E,
K318Q, V372L, G548A, A737S, S819K; H139Q, D184E, N281K, K318Q, V372L,
G548A, A737S, S819K; H139Q, D184E, K318Q, V372L, G548A, W621Q, A737S,
S819K; H139Q, D184E, A262T, K318Q, V372L, G548A, A737S, S819K; H139Q,
D184E, K318Q, V372L, G548A, D631C, A737S, S819K; H139Q, D184E, D289T,
K318Q, V372L, G548A, A737S, S819K; H139Q, D184E, K318Q, V372L, E543L,
G548A, A737S, S819K; H139Q, D184E, K318Q, V372L, G548A, I622K, A737S, S819K;
H139Q, D184E, K318Q, V372L, G548A, L684K, A737S, S819K; H139Q, D184E,
N265R, K318Q, V372L, G548A, A737S, S819K; H139Q, D184E, K318Q, S352C, V372L,
G548A, A737S, S819K; T15K, H106Q, E121N, E131K, H139Q, D184E, H305K, N314K;
E131K, H139Q, D184E, E198K, H305K, M311V, K318Q, S352C; H139Q, D184E,
H305K, M311V, K318Q, S352C, V372L, S547N; H106Q, H139Q, D184E, F200Y,
K318Q, S352C, V372L, G548A; T15K, H106Q, E131K, H139Q, D184E, M311V, K318Q,
V372L; H106Q, H139Q, D184E, M311V, K318Q, S352C, V372L, G548A; T15K, H106Q,
H139Q, D184E, H305K, M311V, K318Q, V372L; H139Q, D184E, F200Y, K318Q,
S352C, V372L, G548A, L647V; H139Q, D184E, E198K, H305K, K318Q, S352C, V372L,
G548A; H139Q, D184E, H305K, M311V, K318Q, S352C, V372L, G548A; H106Q,
E131K, H139Q, D184E, F200Y, M311V, K318Q, V372L; T15K, E121N, E131K, H139Q,
D184E, F200Y, K318Q, V372L; H106Q, H139Q, D184E, M311V, K318Q, V372L,
G548A, D569H; H139Q, D184E, F200Y, K318Q, V372L, G548A, K717M, A737S;
H139Q, D184E, K318Q, V372L, V501T, L537R, G548A, N567S; H139Q, D184E, F200Y,
K318Q, V372L, G548A, D569H, A737S; H139Q, D184E, K318Q, V372L, G548A,
N567S, A737S, D800K; H139Q, D184E, K318Q, V372L, G548A, D569H, A737S,
S807R; H106Q, H139Q, D184E, F200Y, K318Q, S352C, V372L, L537R; H106Q, H139Q,
D184E, F200Y, M311V, K318Q, V372L, G548A; H139Q, D184E, K318Q, V372L,
V501T, G548A, D569H, A737S; H139Q, D184E, K318Q, V372L, G548A, N567S,
A737S, W765N; H139Q, D184E, K318Q, V372L, G548A, A737S, W765N, S807R;
H139Q, D184E, F200Y, K318Q, S352C, V372L, G548A, Q585L; H139Q, D184E,
K318Q, V372L, G548A, A737S, D800K, S819K; H139Q, D184E, M311V, K318Q,
S352C, V372L, V501T, L537R; H139Q, D184E, M311V, K318Q, S352C, V372L,
V492Q, G548A; H139Q, D184E, K318Q, V372L, G548A, A737S, W765N, D800K;
H139Q, D184E, K318Q, V372L, V501T, L537R, G548A, D569H; H139Q, D184E,
F200Y, K318Q, V372L, G548A, A737S, S819K; H139Q, D184E, K318Q, V372L,
G548A, N567S, N722R, A737S*

Exemplary Combinations of 9 Amino Acid Alterations

H139Q, D184E, K318Q, V372L, G548A, A737S, S819K, D974Y, I1064K; H139Q,
D184E, M311V, K318Q, S352C, V372L, V501T, G548A, N722R; H139Q, G154K,
D184E, M311V, K318Q, S352C, V372L, V501T, G548A; H139Q, D184E, M311V,
K318Q, S352C, V372L, V501T, G548A, W621Q; H106Q, E121N, C125L, M311V,
S547N, Y559N, Y858L, E915V, S1062P; H106Q, E121N, M311V, Q480Y, S521A,
Q709H, Y858L, E915V, S1062P; H106Q, F200Y, M311V, Q480Y, S547N, Y559N,
Y858L, M927I, S1062P; H139Q, Q175K, D184E, K318Q, L338I, V372L, G548A, T747L,
N909D; H139Q, D184E, K318Q, V372L, G548A, A737S, S807R, N871K, I1064K;
H139Q, D184E, R247T, K318Q, V372L, G548A, A737S, S819K, D974Y; T9G, H139Q,
D184E, K318Q, V372L, G548A, A737S, S819K, D974Y; H139Q, D184E, K318Q,
V372L, G548A, A737S, S819K, D974Y, Q1056L; H139Q, D184E, K318Q, V372L,
G548A, A737S, S819K, D974Y, Q1054T; H139Q, D184E, A313S, K318Q, V372L,
G548A, A737S, S819K, D974Y; H139Q, N150Y, D184E, K318Q, V372L, G548A,
A737S, S819K, D974Y; H139Q, D184E, K318Q, V372L, G548A, A737S, S819K,
D974Y, H1020R; H139Q, D184E, K318Q, V372L, G548A, I622N, A737S, S819K,
D974Y; H139Q, D184E, K318Q, V372L, G548A, M623L, A737S, S819K, D974Y;
H139Q, Q166K, D184E, K318Q, V372L, G548A, A737S, S819K, D974Y; H139Q,
D184E, K318Q, V372L, G548A, T726Q, A737S, S819K, D974Y; H139Q, D184E,
K318Q, E333H, V372L, G548A, A737S, S819K, D974Y; H139Q, G154K, D184E,
K318Q, V372L, G548A, A737S, S819K, D974Y; H139Q, D184E, K318Q, V372L,
G548A, A737S, A762G, S819K, D974Y; H139Q, D184E, A242M, K318Q, V372L,
G548A, A737S, S819K, D974Y; H139Q, D184E, K318Q, V372L, G548A, A737S,
S819K, N909E, D974Y; H139Q, D184E, K318Q, V372L, G548A, A737S, S819K,
D974Y, D1049G; Q56A, H139Q, D184E, K318Q, V372L, G548A, A737S, S819K,
D974Y; H139Q, D184E, N281K, K318Q, V372L, G548A, A737S, S819K, D974Y;
H139Q, D184E, K318Q, V372L, G548A, W621Q, A737S, S819K, D974Y; H139Q,
D184E, K318Q, V372L, G548A, A737S, S819K, D974Y, T1032V; H139Q, D184E,
K318Q, V372L, G548A, A737S, S819K, D974Y, D1035M; H139Q, D184E, A262T,
K318Q, V372L, G548A, A737S, S819K, D974Y; H139Q, D184E, K318Q, V372L,
G548A, D631C, A737S, S819K, D974Y; H139Q, D184E, D289T, K318Q, V372L,
G548A, A737S, S819K, D974Y; H139Q, D184E, K318Q, V372L, E543L, G548A, A737S,
S819K, D974Y; H139Q, D184E, K318Q, V372L, G548A, I622K, A737S, S819K, D974Y;
H139Q, D184E, K318Q, V372L, G548A, L684K, A737S, S819K, D974Y; H139Q,
D184E, N265R, K318Q, V372L, G548A, A737S, S819K, D974Y; H139Q, D184E,
K318Q, S352C, V372L, G548A, A737S, S819K, S1062P; T15K, H106Q, E121N, E131K,
H139Q, D184E, H305K, N314K, K318Q; E131K, H139Q, D184E, E198K, H305K,
M311V, K318Q, S352C, V372L; H139Q, D184E, K318Q, S352C, V372L, G548A,
A737S, S819K, M927I; H139Q, D184E, H305K, M311V, K318Q, S352C, V372L, S547N,
G548A; H106Q, H139Q, D184E, F200Y, K318Q, S352C, V372L, G548A, A737S; T15K,
H106Q, E131K, H139Q, D184E, M311V, K318Q, V372L, G548A; H106Q, H139Q,
D184E, M311V, K318Q, S352C, V372L, G548A, L647V; T15K, H106Q, H139Q, D184E,
H305K, M311V, K318Q, V372L, G548A; H106Q, H139Q, D184E, M311V, K318Q,
S352C, V372L, G548A, A737S; H139Q, D184E, F200Y, K318Q, S352C, V372L, G548A,
L647V, A737S; H139Q, D184E, E198K, H305K, K318Q, S352C, V372L, G548A, A737S;
H139Q, D184E, H305K, M311V, K318Q, S352C, V372L, G548A, A737S; H106Q,
E131K, H139Q, D184E, F200Y, M311V, K318Q, V372L, G548A; T15K, E121N, E131K,
H139Q, D184E, F200Y, K318Q, V372L, G548A; H106Q, H139Q, D184E, M311V,
K318Q, V372L, G548A, D569H, N722R; H139Q, D184E, F200Y, K318Q, V372L,
G548A, K717M, A737S, D800K; H139Q, D184E, K318Q, V372L, V501T, L537R,
G548A, N567S, A737S; H139Q, D184E, F200Y, K318Q, V372L, G548A, D569H,
A737S, S819K; H139Q, D184E, K318Q, V372L, G548A, N567S, A737S, D800K, S819K;
H139Q, D184E, K318Q, V372L, G548A, D569H, A737S, S807R, S819K; H106Q,
H139Q, D184E, F200Y, K318Q, S352C, V372L, L537R, G548A; H139Q, D184E,
M311V, K318Q, S352C, V372L, V501T, G548A, Q585L; H106Q, H139Q, D184E,
F200Y, M311V, K318Q, V372L, G548A, N567S; H139Q, D184E, K318Q, V372L,
V501T, G548A, D569H, A737S, W765N; H139Q, D184E, K318Q, V372L, G548A,
N567S, A737S, W765N, D800K; H139Q, D184E, K318Q, V372L, G548A, A737S,
W765N, S807R, S819K; H139Q, D184E, F200Y, K318Q, S352C, V372L, G548A,
Q585L, A737S; H106Q, H139Q, D184E, M311V, K318Q, V372L, G548A, D569H,
A737S; H139Q, D184E, K318Q, V372L, G548A, A737S, D800K, S819K, D974Y;
H139Q, D184E, M311V, K318Q, S352C, V372L, V501T, L537R, G548A; H139Q,
D184E, M311V, K318Q, S352C, V372L, V492Q, G548A, Q585L; H139Q, D184E,
K318Q, V372L, G548A, A737S, W765N, D800K, S819K; H139Q, D184E, K318Q,
V372L, V501T, L537R, G548A, D569H, Q585L; H139Q, D184E, F200Y, K318Q,
V372L, G548A, A737S, S819K, D974Y; H139Q, D184E, K318Q, V372L, G548A,
N567S, N722R, A737S, S807R*

Exemplary Combinations of 10 Amino Acid Alterations

H139Q, D184E, M311V, K318Q, S352C, V372L, V501T, G548A, N722R, A737S;
H139Q, G154K, D184E, M311V, K318Q, S352C, V372L, V501T, G548A, I622N;
H139Q, D184E, M311V, K318Q, S352C, V372L, V501T, G548A, W621Q, I622N;
H139Q, Q175K, D184E, K318Q, L338I, V372L, G548A, T747L, N909D, I1064K;
H139Q, D184E, R247T, K318Q, V372L, G548A, A737S, S819K, D974Y, I1064K; T9G,
H139Q, D184E, K318Q, V372L, G548A, A737S, S819K, D974Y, I1064K; H139Q,
D184E, K318Q, V372L, G548A, A737S, S819K, D974Y, Q1056L, I1064K; H139Q,
D184E, K318Q, V372L, G548A, A737S, S819K, D974Y, Q1054T, I1064K; H139Q,
D184E, A313S, K318Q, V372L, G548A, A737S, S819K, D974Y, I1064K; H139Q,
N150Y, D184E, K318Q, V372L, G548A, A737S, S819K, D974Y, I1064K; H139Q,
D184E, K318Q, V372L, G548A, A737S, S819K, D974Y, H1020R, I1064K; H139Q,
D184E, K318Q, V372L, G548A, I622N, A737S, S819K, D974Y, I1064K; H139Q, D184E,
K318Q, V372L, G548A, M623L, A737S, S819K, D974Y, I1064K; H139Q, Q166K,
D184E, K318Q, V372L, G548A, A737S, S819K, D974Y, I1064K; H139Q, D184E,
K318Q, V372L, G548A, T726Q, A737S, S819K, D974Y, I1064K; H139Q, D184E,
K318Q, E333H, V372L, G548A, A737S, S819K, D974Y, I1064K; H139Q, G154K,
D184E, K318Q, V372L, G548A, A737S, S819K, D974Y, I1064K; H139Q, D184E,
K318Q, V372L, G548A, A737S, A762G, S819K, D974Y, I1064K; H139Q, D184E,
A242M, K318Q, V372L, G548A, A737S, S819K, D974Y, I1064K; H139Q, D184E,
K318Q, V372L, G548A, A737S, S819K, N909E, D974Y, I1064K; H139Q, D184E,
K318Q, V372L, G548A, A737S, S819K, D974Y, D1049G, I1064K; Q56A, H139Q,
D184E, K318Q, V372L, G548A, A737S, S819K, D974Y, I1064K; H139Q, D184E,
N281K, K318Q, V372L, G548A, A737S, S819K, D974Y, I1064K; H139Q, D184E,
K318Q, V372L, G548A, W621Q, A737S, S819K, D974Y, I1064K; H139Q, D184E,
K318Q, V372L, G548A, A737S, S819K, D974Y, T1032V, I1064K; H139Q, D184E,
K318Q, V372L, G548A, A737S, S819K, D974Y, D1035M, I1064K; H139Q, D184E,
K318Q, V372L, G548A, A737S, S819K, D974Y, I1064K, P1083Q; H139Q, D184E,
A262T, K318Q, V372L, G548A, A737S, S819K, D974Y, I1064K; H139Q, D184E,
K318Q, V372L, G548A, D631C, A737S, S819K, D974Y, I1064K; H139Q, D184E,
D289T, K318Q, V372L, G548A, A737S, S819K, D974Y, I1064K; H139Q, D184E,
K318Q, V372L, E543L, G548A, A737S, S819K, D974Y, I1064K; H139Q, D184E,
K318Q, V372L, G548A, I622K, A737S, S819K, D974Y, I1064K; H139Q, D184E,
K318Q, V372L, G548A, L684K, A737S, S819K, D974Y, I1064K; H139Q, D184E,
N265R, K318Q, V372L, G548A, A737S, S819K, D974Y, I1064K; T15K, H106Q, E121N,
E131K, H139Q, D184E, H305K, N314K, K318Q, S352C; E131K, H139Q, D184E,
E198K, H305K, M311V, K318Q, S352C, V372L, G548A; H139Q, D184E, K318Q,
S352C, V372L, G548A, A737S, S819K, M927I, A1030S; H139Q, D184E, H305K,
M311V, K318Q, S352C, V372L, S547N, G548A, L647V; H106Q, H139Q, D184E,
F200Y, K318Q, S352C, V372L, G548A, A737S, I1064K; T15K, H106Q, E131K, H139Q,
D184E, M311V, K318Q, V372L, G548A, A737S; H106Q, H139Q, D184E, M311V,
K318Q, S352C, V372L, G548A, L647V, A737S; T15K, H106Q, H139Q, D184E, H305K,
M311V, K318Q, V372L, G548A, A737S; H106Q, H139Q, D184E, M311V, K318Q,
S352C, V372L, G548A, A737S, S819K; H139Q, D184E, F200Y, K318Q, S352C, V372L,
G548A, L647V, A737S, Y858L; H139Q, D184E, E198K, H305K, K318Q, S352C, V372L,
G548A, A737S, S819K; H139Q, D184E, H305K, M311V, K318Q, S352C, V372L,
G548A, A737S, S819K; H106Q, E131K, H139Q, D184E, F200Y, M311V, K318Q,
V372L, G548A, A737S; T15K, E121N, E131K, H139Q, D184E, F200Y, K318Q, V372L,
G548A, A737S; H106Q, H139Q, D184E, M311V, K318Q, V372L, G548A, D569H,
N722R, A737S; H139Q, D184E, F200Y, K318Q, V372L, G548A, K717M, A737S,
D800K, S819K; H139Q, D184E, K318Q, V372L, V501T, L537R, G548A, N567S, A737S,
S807R; H139Q, D184E, F200Y, K318Q, V372L, G548A, D569H, A737S, S819K,
D974Y; H139Q, D184E, K318Q, V372L, G548A, N567S, A737S, D800K, S819K,
D974Y; H139Q, D184E, K318Q, V372L, G548A, D569H, A737S, S807R, S819K,
S919K; H106Q, H139Q, D184E, F200Y, K318Q, S352C, V372L, L537R, G548A, N567S;
H139Q, D184E, M311V, K318Q, S352C, V372L, V501T, G548A, Q585L, A705T;
H106Q, H139Q, D184E, F200Y, M311V, K318Q, V372L, G548A, N567S, D569H;
H139Q, D184E, K318Q, V372L, V501T, G548A, D569H, A737S, W765N, S807R;
H139Q, D184E, K318Q, V372L, G548A, N567S, A737S, W765N, D800K, S807R;
H139Q, D184E, K318Q, V372L, G548A, A737S, W765N, S807R, S819K, D974Y;
H139Q, D184E, F200Y, K318Q, S352C, V372L, G548A, Q585L, A737S, S819K; H106Q,
H139Q, D184E, M311V, K318Q, V372L, G548A, D569H, A737S, D800K; H139Q,
D184E, K318Q, V372L, G548A, A737S, D800K, S819K, D974Y, L1011V; H139Q,
D184E, M311V, K318Q, S352C, V372L, V501T, L537R, G548A, A737S; H139Q,
D184E, M311V, K318Q, S352C, V372L, V492Q, G548A, Q585L, N722R; H139Q,
D184E, K318Q, V372L, G548A, A737S, W765N, D800K, S819K, D974Y; H139Q,
D184E, K318Q, V372L, V501T, L537R, G548A, D569H, Q585L, A737S; H139Q,
D184E, F200Y, K318Q, V372L, G548A, A737S, S819K, D974Y, I1064K; H139Q,
D184E, K318Q, V372L, G548A, N567S, N722R, A737S, S807R, S819K*

Exemplary Combinations of 11 Amino Acid Alterations

H139Q, D184E, M311V, K318Q, S352C, V372L, V501T, G548A, N722R, A737S,
W765N; H139Q, G154K, D184E, M311V, K318Q, S352C, V372L, V501T, G548A,
I622N, N722R; H139Q, D184E, M311V, K318Q, S352C, V372L, V501T, G548A,
W621Q, I622N, N722R; H139Q, Q175K, D184E, K318Q, L338I, V372L, G548A, T747L,
N909D, I1064K, A1085E; T15K, H106Q, E121N, E131K, H139Q, D184E, H305K,
N314K, K318Q, S352C, V372L; E131K, H139Q, D184E, E198K, H305K, M311V,
K318Q, S352C, V372L, G548A, A737S; H139Q, D184E, K318Q, S352C, V372L, G548A,
A737S, S819K, M927I, A1030S, ; H139Q, D184E, H305K, M311V, K318Q, S352C,
V372L, S547N, G548A, L647V, A737S; T15K, H106Q, E131K, H139Q, D184E, M311V,
K318Q, V372L, G548A, A737S, I923L; H106Q, H139Q, D184E, M311V, K318Q, S352C,
V372L, G548A, L647V, A737S, S819K; T15K, H106Q, H139Q, D184E, H305K, M311V,
K318Q, V372L, G548A, A737S, I923L; H106Q, H139Q, D184E, M311V, K318Q, S352C,
V372L, G548A, A737S, S819K, I1064K; H139Q, D184E, F200Y, K318Q, S352C, V372L,
G548A, L647V, A737S, Y858L, A1030S; H139Q, D184E, H305K, M311V, K318Q,
S352C, V372L, G548A, A737S, S819K, S1062P; H106Q, E131K, H139Q, D184E,
F200Y, M311V, K318Q, V372L, G548A, A737S, ; T15K, E121N, E131K, H139Q,
D184E, F200Y, K318Q, V372L, G548A, A737S, I1064K; H106Q, H139Q, D184E,
M311V, K318Q, V372L, G548A, D569H, N722R, A737S, S807R; H139Q, D184E,
F200Y, K318Q, V372L, G548A, K717M, A737S, D800K, S819K, N871K; H139Q,
D184E, K318Q, V372L, V501T, L537R, G548A, N567S, A737S, S807R, S819K; H139Q,
D184E, F200Y, K318Q, V372L, G548A, D569H, A737S, S819K, D974Y, I1064K;
H139Q, D184E, K318Q, V372L, G548A, N567S, A737S, D800K, S819K, D974Y,
I1064K; H139Q, D184E, K318Q, V372L, G548A, D569H, A737S, S807R, S819K,
S919K, D974Y; H106Q, H139Q, D184E, F200Y, K318Q, S352C, V372L, L537R, G548A,
N567S, A737S; H139Q, D184E, M311V, K318Q, S352C, V372L, V501T, G548A,
Q585L, A705T, N722R; H106Q, H139Q, D184E, F200Y, M311V, K318Q, V372L,
G548A, N567S, D569H, A737S; H139Q, D184E, K318Q, V372L, V501T, G548A,
D569H, A737S, W765N, S807R, S819K; H139Q, D184E, K318Q, V372L, G548A,
N567S, A737S, W765N, D800K, S807R, S819K; H139Q, D184E, K318Q, V372L,
G548A, A737S, W765N, S807R, S819K, D974Y, I1064K; H139Q, D184E, F200Y,
K318Q, S352C, V372L, G548A, Q585L, A737S, S819K, D974Y; H106Q, H139Q,
D184E, M311V, K318Q, V372L, G548A, D569H, A737S, D800K, S807R; H139Q,
D184E, K318Q, V372L, G548A, A737S, D800K, S819K, D974Y, L1011V, I1064K;
H139Q, D184E, M311V, K318Q, S352C, V372L, V501T, L537R, G548A, A737S,
D800K; H139Q, D184E, M311V, K318Q, S352C, V372L, V492Q, G548A, Q585L,
N722R, A737S; H139Q, D184E, K318Q, V372L, G548A, A737S, W765N, D800K,
S819K, D974Y, I1064K; H139Q, D184E, K318Q, V372L, V501T, L537R, G548A,
D569H, Q585L, A737S, W765N; H139Q, D184E, K318Q, V372L, G548A, N567S,
N722R, A737S, S807R, S819K, N843S*

Exemplary Combinations of 12 Amino Acid Alterations

H139Q, D184E, M311V, K318Q, S352C, V372L, V501T, G548A, N722R, A737S,
W765N, S807R; H139Q, G154K, D184E, M311V, K318Q, S352C, V372L, V501T,
G548A, I622N, N722R, A737S; H139Q, D184E, M311V, K318Q, S352C, V372L, V501T,
G548A, W621Q, I622N, N722R, A737S; T15K, H106Q, E121N, E131K, H139Q, D184E,
H305K, N314K, K318Q, S352C, V372L, G548A; E131K, H139Q, D184E, E198K,
H305K, M311V, K318Q, S352C, V372L, G548A, A737S, S819K; H139Q, D184E,
H305K, M311V, K318Q, S352C, V372L, S547N, G548A, L647V, A737S, S819K;
H106Q, H139Q, D184E, M311V, K318Q, S352C, V372L, G548A, L647V, A737S,
S819K, A1030S; T15K, H106Q, H139Q, D184E, H305K, M311V, K318Q, V372L,
G548A, A737S, I923L, S1062P; H139Q, D184E, F200Y, K318Q, S352C, V372L, G548A,
L647V, A737S, Y858L, A1030S, S1062P; H106Q, H139Q, D184E, M311V, K318Q,
V372L, G548A, D569H, N722R, A737S, S807R, S819K; H139Q, D184E, F200Y, K318Q,
V372L, G548A, K717M, A737S, D800K, S819K, N871K, D974Y; H139Q, D184E,
K318Q, V372L, V501T, L537R, G548A, N567S, A737S, S807R, S819K, D974Y; H139Q,
D184E, K318Q, V372L, G548A, D569H, A737S, S807R, S819K, S919K, D974Y,
I1064K; H106Q, H139Q, D184E, F200Y, K318Q, S352C, V372L, L537R, G548A,
N567S, A737S, D800K; H139Q, D184E, M311V, K318Q, S352C, V372L, V501T,
G548A, Q585L, A705T, N722R, A737S; H106Q, H139Q, D184E, F200Y, M311V,
K318Q, V372L, G548A, N567S, D569H, A737S, S819K; H139Q, D184E, K318Q,
V372L, V501T, G548A, D569H, A737S, W765N, S807R, S819K, N871K; H139Q,
D184E, K318Q, V372L, G548A, N567S, A737S, W765N, D800K, S807R, S819K,
S919K; H139Q, D184E, F200Y, K318Q, S352C, V372L, G548A, Q585L, A737S, S819K,
D974Y, I1064K; H106Q, H139Q, D184E, M311V, K318Q, V372L, G548A, D569H,
A737S, D800K, S807R, S819K; H139Q, D184E, M311V, K318Q, S352C, V372L, V501T,
L537R, G548A, A737S, D800K, S807R; H139Q, D184E, M311V, K318Q, S352C,
V372L, V492Q, G548A, Q585L, N722R, A737S, D800K; H139Q, D184E, K318Q,
V372L, V501T, L537R, G548A, D569H, Q585L, A737S, W765N, S807R; H139Q,
D184E, K318Q, V372L, G548A, N567S, N722R, A737S, S807R, S819K, N843S, N871K*

Exemplary Combinations of 13 Amino Acid Alterations

H139Q, D184E, M311V, K318Q, S352C, V372L, V501T, G548A, N722R, A737S,
W765N, S807R, S819K; H139Q, G154K, D184E, M311V, K318Q, S352C, V372L,
V501T, G548A, I622N, N722R, A737S, W765N; H139Q, D184E, M311V, K318Q,
S352C, V372L, V501T, G548A, W621Q, I622N, N722R, A737S, A762G; T15K, H106Q,
E121N, E131K, H139Q, D184E, H305K, N314K, K318Q, S352C, V372L, G548A,
M656L; E131K, H139Q, D184E, E198K, H305K, M311V, K318Q, S352C, V372L,
G548A, A737S, S819K, Y858L; H139Q, D184E, H305K, M311V, K318Q, S352C,
V372L, S547N, G548A, L647V, A737S, S819K, Y858L; H106Q, H139Q, D184E,
M311V, K318Q, V372L, G548A, D569H, N722R, A737S, S807R, S819K, D974Y;
H139Q, D184E, F200Y, K318Q, V372L, G548A, K717M, A737S, D800K, S819K,
N871K, D974Y, I1064K; H139Q, D184E, K318Q, V372L, V501T, L537R, G548A,
N567S, A737S, S807R, S819K, D974Y, I1064K; H106Q, H139Q, D184E, F200Y,
K318Q, S352C, V372L, L537R, G548A, N567S, A737S, D800K, S819K; H139Q, D184E,
M311V, K318Q, S352C, V372L, V501T, G548A, Q585L, A705T, N722R, A737S,
W765N; H106Q, H139Q, D184E, F200Y, M311V, K318Q, V372L, G548A, N567S,
D569H, A737S, S819K, D974Y; H139Q, D184E, K318Q, V372L, V501T, G548A,
D569H, A737S, W765N, S807R, S819K, N871K, D974Y; H139Q, D184E, K318Q,
V372L, G548A, N567S, A737S, W765N, D800K, S807R, S819K, S919K, D974Y;
H106Q, H139Q, D184E, M311V, K318Q, V372L, G548A, D569H, A737S, D800K,
S807R, S819K, S919K; H139Q, D184E, M311V, K318Q, S352C, V372L, V501T, L537R,
G548A, A737S, D800K, S807R, S819K; H139Q, D184E, M311V, K318Q, S352C,
V372L, V492Q, G548A, Q585L, N722R, A737S, D800K, S807R; H139Q, D184E,
K318Q, V372L, V501T, L537R, G548A, D569H, Q585L, A737S, W765N, S807R,
S819K; H139Q, D184E, K318Q, V372L, G548A, N567S, N722R, A737S, S807R, S819K,
N843S, N871K, D974Y*

Exemplary Combinations of 14 Amino Acid Alterations

H139Q, D184E, M311V, K318Q, S352C, V372L, V501T, G548A, N722R, A737S,
W765N, S807R, S819K, N871K; H139Q, G154K, D184E, M311V, K318Q, S352C,
V372L, V501T, G548A, I622N, N722R, A737S, W765N, S807R; H139Q, D184E,
M311V, K318Q, S352C, V372L, V501T, G548A, W621Q, I622N, N722R, A737S,
A762G, W765N; T15K, H106Q, E121N, E131K, H139Q, D184E, H305K, N314K,
K318Q, S352C, V372L, G548A, M656L, A737S; E131K, H139Q, D184E, E198K,
H305K, M311V, K318Q, S352C, V372L, G548A, A737S, S819K, Y858L, S1062P;
H106Q, H139Q, D184E, M311V, K318Q, V372L, G548A, D569H, N722R, A737S,
S807R, S819K, D974Y, I1064K; H106Q, H139Q, D184E, F200Y, K318Q, S352C,
V372L, L537R, G548A, N567S, A737S, D800K, S819K, D974Y; H139Q, D184E,
M311V, K318Q, S352C, V372L, V501T, G548A, Q585L, A705T, N722R, A737S,
W765N, S807R; H106Q, H139Q, D184E, F200Y, M311V, K318Q, V372L, G548A,
N567S, D569H, A737S, S819K, D974Y, I1064K; H139Q, D184E, K318Q, V372L,
V501T, G548A, D569H, A737S, W765N, S807R, S819K, N871K, D974Y, I1064K;
H139Q, D184E, K318Q, V372L, G548A, N567S, A737S, W765N, D800K, S807R,
S819K, S919K, D974Y, I1064K; H106Q, H139Q, D184E, M311V, K318Q, V372L,
G548A, D569H, A737S, D800K, S807R, S819K, S919K, D974Y; H139Q, D184E,
M311V, K318Q, S352C, V372L, V501T, L537R, G548A, A737S, D800K, S807R, S819K,
D974Y; H139Q, D184E, M311V, K318Q, S352C, V372L, V492Q, G548A, Q585L,
N722R, A737S, D800K, S807R, S819K; H139Q, D184E, K318Q, V372L, V501T, L537R,
G548A, D569H, Q585L, A737S, W765N, S807R, S819K, S919K; H139Q, D184E,
K318Q, V372L, G548A, N567S, N722R, A737S, S807R, S819K, N843S, N871K, D974Y,
I1064K*

Exemplary Combinations of 15 Amino Acid Alterations

H139Q, D184E, M311V, K318Q, S352C, V372L, V501T, G548A, N722R, A737S,
W765N, S807R, S819K, N871K, D974Y; H139Q, G154K, D184E, M311V, K318Q,
S352C, V372L, V501T, G548A, I622N, N722R, A737S, W765N, S807R, S819K; H139Q,
D184E, M311V, K318Q, S352C, V372L, V501T, G548A, W621Q, I622N, N722R,
A737S, A762G, W765N, D800R; T15K, H106Q, E121N, E131K, H139Q, D184E, H305K,
N314K, K318Q, S352C, V372L, G548A, M656L, A737S, S819K; H106Q, H139Q,
D184E, F200Y, K318Q, S352C, V372L, L537R, G548A, N567S, A737S, D800K, S819K,
D974Y, I1064K; H139Q, D184E, M311V, K318Q, S352C, V372L, V501T, G548A,
Q585L, A705T, N722R, A737S, W765N, S807R, S819K; H106Q, H139Q, D184E,
M311V, K318Q, V372L, G548A, D569H, A737S, D800K, S807R, S819K, S919K,
D974Y, I1064K; H139Q, D184E, M311V, K318Q, S352C, V372L, V501T, L537R,
G548A, A737S, D800K, S807R, S819K, D974Y, I1064K; H139Q, D184E, M311V,
K318Q, S352C, V372L, V492Q, G548A, Q585L, N722R, A737S, D800K, S807R, S819K,
S919K; H139Q, D184E, K318Q, V372L, V501T, L537R, G548A, D569H, Q585L, A737S,
W765N, S807R, S819K, S919K, D974Y*

Exemplary Combinations of 16 Amino Acid Alterations

H139Q, D184E, M311V, K318Q, S352C, V372L, V501T, G548A, N722R, A737S,
W765N, S807R, S819K, N871K, D974Y, I1064K; H139Q, G154K, D184E, M311V,
K318Q, S352C, V372L, V501T, G548A, I622N, N722R, A737S, W765N, S807R, S819K,
S827K; H139Q, D184E, M311V, K318Q, S352C, V372L, V501T, G548A, W621Q,
I622N, N722R, A737S, A762G, W765N, D800R, S807R; T15K, H106Q, E121N, E131K,
H139Q, D184E, H305K, N314K, K318Q, S352C, V372L, G548A, M656L, A737S,
S819K, S1062P; H139Q, D184E, M311V, K318Q, S352C, V372L, V501T, G548A,
Q585L, A705T, N722R, A737S, W765N, S807R, S819K, D974Y; H139Q, D184E,
M311V, K318Q, S352C, V372L, V492Q, G548A, Q585L, N722R, A737S, D800K,
S807R, S819K, S919K, D974Y; H139Q, D184E, K318Q, V372L, V501T, L537R, G548A,
D569H, Q585L, A737S, W765N, S807R, S819K, S919K, D974Y, I1064K*

Exemplary Combinations of 17 Amino Acid Alterations

H139Q, G154K, D184E, M311V, K318Q, S352C, V372L, V501T, G548A, I622N,
N722R, A737S, W765N, S807R, S819K, S827K, N871K; H139Q, D184E, M311V,
K318Q, S352C, V372L, V501T, G548A, W621Q, I622N, N722R, A737S, A762G,
W765N, D800R, S807R, S819K; H139Q, D184E, M311V, K318Q, S352C, V372L,
V501T, G548A, Q585L, A705T, N722R, A737S, W765N, S807R, S819K, D974Y,
I1064K; H139Q, D184E, M311V, K318Q, S352C, V372L, V492Q, G548A, Q585L,
N722R, A737S, D800K, S807R, S819K, S919K, D974Y, I1064K; H139Q, D184E,
M311V, K318Q, S352C, V372L, V501T, G548A, I622N, N722R, A737S, W765N,
S807R, S819K, N871K, D974Y, I1064K; H139Q, D184E, M311V, K318Q, S352C,
V372L, V501T, G548A, N722R, A737S, W765N, S807R, S819K, S827K, N871K,
D974Y, I1064K; H139Q, D184E, M311V, K318Q, S352C, V372L, V501T, G548A,
N722R, A737S, W765N, S807R, S819K, N871K, D974Y, H1020R, I1064K; H139Q,
D184E, M311V, K318Q, S352C, V372L, V501T, G548A, N722R, A737S, W765N,
S807R, S819K, S827K, N871K, D974Y, I1064K; H139Q, G154K, D184E, M311V,
K318Q, S352C, V372L, V501T, G548A, N722R, A737S, W765N, S807R, S819K,
N871K, D974Y, I1064K; H139Q, D184E, M311V, K318Q, S352C, V372L, V501T,
G548A, I622N, N722R, A737S, W765N, S807R, S819K, N871K, D974Y, I1064K*

Exemplary Combinations of 18 Amino Acid Alterations

H139Q, G154K, D184E, M311V, K318Q, S352C, V372L, V501T, G548A, I622N,
N722R, A737S, W765N, S807R, S819K, S827K, N871K, D974Y; H139Q, D184E,
M311V, K318Q, S352C, V372L, V501T, G548A, W621Q, I622N, N722R, A737S,
A762G, W765N, D800R, S807R, S819K, N871K; H139Q, D184E, M311V, K318Q,
S352C, V372L, V501T, G548A, N722R, A737S, A762G, W765N, S807R, S819K, S827K,
N871K, D974Y, I1064K; H139Q, D184E, M311V, K318Q, S352C, V372L, V501T,
E543L, G548A, N722R, A737S, W765N, S807R, S819K, S827K, N871K, D974Y,
I1064K; H139Q, D184E, M311V, K318Q, S352C, V372L, V501T, G548A, I622N,
N722R, A737S, A762G, W765N, S807R, S819K, N871K, D974Y, I1064K; H139Q,
D184E, M311V, K318Q, S352C, V372L, V501T, G548A, N722R, A737S, W765N,
D800R, S807R, S819K, S827K, N871K, D974Y, I1064K; H139Q, D184E, M311V,
K318Q, S352C, V372L, V501T, G548A, T726Q, N722R, A737S, A762G, W765N,
S807R, S819K, N871K, D974Y, I1064K; H106Q, H139Q, D184E, M311V, K318Q,
S352C, V372L, V501T, G548A, N722R, A737S, W765N, S807R, S819K, N871K,
D974Y, H1020R, I1064K; H139Q, D184E, M311V, K318Q, S352C, V372L, V501T,
G548A, N722R, A737S, A762G, W765N, S807R, S819K, S827K, N871K, D974Y,
I1064K; H139Q, D184E, M311V, K318Q, S352C, V372L, V501T, E543L, G548A,
T726Q, N722R, A737S, W765N, S807R, S819K, N871K, D974Y, I1064K*

Exemplary Combinations of 19 Amino Acid Alterations

H139Q, G154K, D184E, M311V, K318Q, S352C, V372L, V501T, G548A, I622N,
N722R, A737S, W765N, S807R, S819K, S827K, N871K, D974Y, H1020R; H139Q,
D184E, M311V, K318Q, S352C, V372L, V501T, G548A, W621Q, I622N, N722R,
A737S, A762G, W765N, D800R, S807R, S819K, N871K, D974Y; H139Q, G154K,
D184E, M311V, K318Q, S352C, V372L, V501T, G548A, T726Q, N722R, A737S,
W765N, S807R, S819K, N871K, D974Y, H1020R, I1064K; H139Q, D184E, M311V,
K318Q, S352C, V372L, V501T, E543L, G548A, N722R, A737S, W765N, S807R, S819K,
S827K, N871K, D974Y, T1032V, I1064K; H106Q, H139Q, D184E, A262T, M311V,
K318Q, S352C, V372L, V501T, G548A, N722R, A737S, A762G, W765N, S807R,
S819K, N871K, D974Y, I1064K; H106Q, H139Q, D184E, M311V, K318Q, S352C,
V372L, V501T, G548A, N722R, A737S, A762G, W765N, S807R, S819K, N871K,
D974Y, T1032V, I1064K; H139Q, G154K, D184E, M311V, K318Q, S352C, V372L,
V501T, G548A, N722R, A737S, W765N, D800R, S807R, S819K, N871K, D974Y,
H1020R, I1064K*

Exemplary Combinations of 20 Amino Acid Alterations

H139Q, G154K, D184E, M311V, K318Q, S352C, V372L, V501T, G548A, I622N,
N722R, A737S, W765N, S807R, S819K, S827K, N871K, D974Y, H1020R, I1064K;
H139Q, D184E, M311V, K318Q, S352C, V372L, V501T, G548A, W621Q, I622N,
N722R, A737S, A762G, W765N, D800R, S807R, S819K, N871K, D974Y, I1064K;
H106Q, H139Q, G154K, D184E, M311V, K318Q, S352C, V372L, V501T, G548A,
N722R, A737S, A762G, W765N, S807R, S819K, N871K, D974Y, D1049G, I1064K;
H106Q, H139Q, D184E, M311V, K318Q, S352C, V372L, V501T, G548A, I622N,
N722R, A737S, W765N, S807R, S819K, S827K, N871K, D974Y, H1020R, I1064K;
H139Q, D184E, M311V, K318Q, S352C, V372L, V501T, E543L, G548A, I622N, N722R,
A737S, W765N, D800R, S807R, S819K, S827K, N871K, D974Y, I1064K; H106Q,
H139Q, G154K, D184E, M311V, K318Q, S352C, V372L, V501T, G548A, T726Q,
N722R, A737S, W765N, D800R, S807R, S819K, N871K, D974Y, I1064K; H139Q,
G154K, D184E, A262T, M311V, K318Q, S352C, V372L, V501T, G548A, T726Q,
N722R, A737S, W765N, S807R, S819K, N871K, D974Y, T1032V, I1064K; H139Q,
D184E, A262T, M311V, K318Q, S352C, V372L, V501T, G548A, W621Q, N722R,
A737S, W765N, D800R, S807R, S819K, S827K, N871K, D974Y, I1064K; H139Q,
G154K, D184E, M311V, K318Q, S352C, V372L, V501T, G548A, N722R, A737S,
W765N, D800R, S807R, S819K, S827K, N871K, D974Y, T1032V, I1064K; H139Q,
G154K, D184E, M311V, K318Q, S352C, V372L, V501T, G548A, N722R, A737S,
W765N, D800R, S807R, S819K, S827K, N871K, D974Y, T1032V, I1064K; H139Q,
D184E, M311V, K318Q, S352C, V372L, V501T, G548A, W621Q, N722R, A737S,
W765N, D800R, S807R, S819K, S827K, N871K, D974Y, H1020R, I1064K; H106Q,
H139Q, G154K, D184E, M311V, K318Q, S352C, V372L, V501T, G548A, N722R,
A737S, A762G, W765N, S807R, S819K, N871K, D974Y, H1020R, I1064K; H139Q,
G154K, D184E, M311V, K318Q, S352C, V372L, V501T, E543L, G548A, T726Q,
N722R, A737S, W765N, S807R, S819K, N871K, D974Y, H1020R, I1064K*

Exemplary Combinations of 21 Amino Acid Alterations

H106Q, H139Q, G154K, D184E, M311V, K318Q, S352C, V372L, V501T, G548A,
W621Q, N722R, A737S, A762G, W765N, S807R, S819K, S827K, N871K, D974Y,
I1064K; H106Q, H139Q, G154K, D184E, M311V, K318Q, S352C, V372L, V501T,
G548A, L684K, N722R, A737S, W765N, S807R, S819K, S827K, N871K, D974Y,
H1020R, I1064K; H106Q, H139Q, G154K, D184E, M311V, K318Q, S352C, V372L,
V501T, G548A, W621Q, N722R, A737S, A762G, W765N, S807R, S819K, S827K,
N871K, D974Y, I1064K; H139Q, D184E, M311V, K318Q, S352C, V372L, V501T,
E543L, G548A, I622N, N722R, A737S, W765N, D800R, S807R, S819K, S827K, N871K,
S919K, D974Y, I1064K; H106Q, H139Q, D184E, A262T, M311V, K318Q, S352C,
V372L, V501T, G548A, T726Q, N722R, A737S, W765N, S807R, S819K, N871K,
D974Y, T1032V, H1020R, I1064K*

Exemplary Combinations of 22 Amino Acid Alterations

H139Q, D184E, A262T, M311V, K318Q, S352C, V372L, V501T, E543L, G548A, I622N,
N722R, A737S, W765N, D800R, S807R, S819K, S827K, N871K, D974Y, H1020R,
I1064K; H106Q, H139Q, G154K, D184E, M311V, K318Q, S352C, V372L, V501T,
G548A, I622N, T726Q, N722R, A737S, W765N, S807R, S819K, S827K, N871K, D974Y,
D1049G, I1064K*

Exemplary Combinations of 23 Amino Acid Alterations

H106Q, H139Q, D184E, A262T, M311V, K318Q, S352C, V372L, V501T, G548A,
I622N, T726Q, N722R, A737S, A762G, W765N, S807R, S819K, S827K, N871K, D974Y,
H1020R, I1064K*
*wherein “,” indicates simultaneous substitutions

TABLE 1.2 provides illustrative amino acid sequences of exemplary variants of effector proteins that are useful in the compositions, systems and methods described herein.

TABLE 1.2
Sequences of Exemplary Variant Amino Acid Sequence relative to SEQ ID NO: 69

| SEQ ID | | |
|---|---|---|
| NO: | Variant | Amino Acid Sequence |
| 119 | E121N- | MRVSKVKVTVNNKDTMVLMHRSAEKGALIYDTIDKKDRTEEIIPQKKQ |
| | M311V- | KSFELSIQNKTLLRYEAIKKQKLDRSKYKVVEDIFKYRKLPAKITLEDILP |
| | S547N- | FLNHKFHLPVKYQKEGQWVQFNLSTCILQAVEHQNLDELHPYYDWSKW |
| | M656L- | YINDKSGKLKKSITNNHIQIGDGESKRQKALLMWETDFTDKGELDLETY |
| | Y858L- | HEKFIIDTLAASLRSVEKGELRDNGTPKETNSYHRNLKKVLQNHQALIFG |
| | M927I- | RRETPNQENRDNKQLAIYNLEVVKYLEHYFPIKSNVRKNSAADIVYYLK |
| | S1062P | SETIKSTVTHQLENAVRANLLRKGKYQHHDLKADATSENLTNLKRDEAF |
| | | VMNLITTSAFAANNIRNIIDPEQIKDIVGKDDFIRSLKKDAFNKHIYGLFFP |
| | | VGAEMIEPVSLWAMRGAVQQIRNNVAHYKKDALATIFNITKFEYPGAEN |
| | | ILFSNTIYKQFFEKELEQLPEAFALQLKSGGVLAQYGFDQLKFLLDVVKF |
| | | HLCRTVVPFAPGFKKVMKGGTGYQNSKQDESFYNLELTAYQLKDDFSE |
| | | EAWNGRYFLLKIIYNYLFLPEFTNNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFLKQSAYQFINTPQPQINPTDTNQQQADKLKALEKK |
| | | ICPFCHIQNNKIDPAKDEQIAFYVYCKLLDANHLSTLRNELIKYRAASESE |
| | | LFIHLTEIIELCLLHADNVPADYWQIYKNKEQCLQRLQPFIAENSDYTNW |
| | | GDLYVQTDDETPVVHSGIELSVKYGTASLLEKLIASDAKFKITENEFDLW |
| | | HNARATIEAMIKKREELHNLWLEAKEKDDNEKKTRIREKNNYARRFVET |
| | | KGEDYIDICDYIDRYNWLNNKLHFEHLKSLQNITIEILGRMAGFVVLWER |
| | | DFQYLDQQRVKQQGGALMDFSHGVPKYEELKPHDAYFKRLFLCNNYSD |
| | | TRNHIAHFNYLTKSAANYSLIDLINQLRELLHYDRKLKNAVAKTFIDLFD |
| | | KHGMVLKLKFDSQMHQLQVGSITPKIIFHLGTKKESDNIYTFQVPTAYCN |
| | | MCKQLLEMKN |
| 120 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | G548A- | HEKFIIDTLAASLRSVEKGELRDNGTPKETNSYHRNLKKVLQNHQALIFG |
| | A737S | RRETPNQENRDNKQLAIYNLEVVKYLEHYFPIKSNVRKNSAADIVYYLK |
| | | SETIKSTVTHQLENAMRANLLRQGKYQHHDLKADATSENLTNLKRDEA |
| | | FVMNLITTSAFAANNIRNIIDPEQIKDILGKDDFIRSLKKDAFNKHIYGLFF |
| | | PVGAEMIEPVSLWAMRGAVQQIRNNVAHYKKDALATIFNITKFEYPGAE |
| | | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFINNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDDETPVVHSGIELSVKYGTASLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHDAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKIIFHLGTKKESDNIYTFQVPT |
| | | AYCNMCKQLLEMKN |
| 121 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | G548A- | HEKFIIDTLAASLRSVEKGELRDNGTPKETNSYHRNLKKVLQNHQALIFG |
| | A737S- | RRETPNQENRDNKQLAIYNLEVVKYLEHYFPIKSNVRKNSAADIVYYLK |
| | S819K- | SETIKSTVTHQLENAMRANLLRQGKYQHHDLKADATSENLTNLKRDEA |
| | D974Y- | FVMNLITTSAFAANNIRNIIDPEQIKDILGKDDFIRSLKKDAFNKHIYGLFF |
| | I1064K | PVGAEMIEPVSLWAMRGAVQQIRNNVAHYKKDALATIFNITKFEYPGAE |
| | | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFINNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDDETPVVHSGIELSVKYGTAKLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHYAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 122 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | M311V- | FLNHKFHLPVKYQKEGQWVQFELSTCILQAVEHQNLDELQPYYDWSKW |
| | K318Q- | YINDKSGKLKKSITNNHIQIGDGESKRQKALLMWETEFTDKGELDLETY |
| | S352C- | HEKFIIDTLAASLRSVEKGELRDNGTPKETNSYHRNLKKVLQNHQALIFG |
| | V372L- | RRETPNQENRDNKQLAIYNLEVVKYLEHYFPIKSNVRKNSAADIVYYLK |
| | V501T- | SETIKSTVTHQLENAVRANLLRQGKYQHHDLKADATSENLTNLKRDEAF |
| | G548A- | VMNLITTCAFAANNIRNIIDPEQIKDILGKDDFIRSLKKDAFNKHIYGLFFP |
| | N722R- | VGAEMIEPVSLWAMRGAVQQIRNNVAHYKKDALATIFNITKFEYPGAEN |
| | A737S- | ILFSNTIYKQFFEKELEQLPEAFALQLKSGGVLAQYGFDQLKFLLDVVKF |
| | W765N- | HLCRTTVPFAPGFKKVMKGGTGYQNSKQDESFYNLELTAYQLKDDFSEE |
| | S807R- | AWSARYFLLKIIYNYLFLPEFTNNDKVFADTVAWLLGKNRQQAQSSKNK |
| | S819K- | NAYAFADIRLMESSESITDYMAYVQSQWIMEENKKEDDKKEDTRINFEK |
| | N871K- | FVLQLFVKGFDTFMKQSAYQFINTPQPQINPTDTNQQQADKLKALEKKIC |
| | D974Y- | PFCHIQNNKIDPAKDEQIAFYVYCKLLDARHLSTLRNELIKYRASSESELFI |
| | I1064K | HLTEIIELCLLHADNVPADYNQIYKNKEQCLQRLQPFIAENSDYTNWGDL |
| | | YVQTDDETPVVHRGIELSVKYGTAKLLEKLIASDAKFKITENEFDLWHN |
| | | ARATIEAMIKKREEYHNLWLEAKEKDDKEKKTRIREKNNYARRFVETK |
| | | GEDYIDICDYIDRYNWLNNKLHFEHLKSLQNITIEMLGRMAGFVVLWER |
| | | DFQYLDQQRVKQQGGALMDFSHGVPKYEELKPHYAYFKRLFLCNNYSD |
| | | TRNHIAHFNYLTKSAANYSLIDLINQLRELLHYDRKLKNAVAKTFIDLFD |
| | | KHGMVLKLKFDSQMHQLQVGSITSKKIFHLGTKKESDNIYTFQVPTAYC |
| | | NMCKQLLEMKN |
| 123 | H139Q- | MRVSKVKVTVNNKDTMVLMHRSAEKGALIYDTIDKKDRTEEIIPQKKQ |
| | G154K- | KSFELSIQNKTLLRYEAIKKQKLDRSKYKVVEDIFKYRKLPAKITLEDILP |
| | D184E- | FLNHKFHLPVKYQKEGQWVQFELSTCILQAVEHQNLDELQPYYDWSKW |
| | M311V- | YINDKSKKLKKSITNNHIQIGDGESKRQKALLMWETEFTDKGELDLETY |
| | K318Q- | HEKFIIDTLAASLRSVEKGELRDNGTPKETNSYHRNLKKVLQNHQALIFG |
| | S352C- | RRETPNQENRDNKQLAIYNLEVVKYLEHYFPIKSNVRKNSAADIVYYLK |
| | V372L- | SETIKSTVTHQLENAVRANLLRQGKYQHHDLKADATSENLTNLKRDEAF |
| | V501T- | VMNLITTCAFAANNIRNIIDPEQIKDILGKDDFIRSLKKDAFNKHIYGLFFP |
| | G548A- | VGAEMIEPVSLWAMRGAVQQIRNNVAHYKKDALATIFNITKFEYPGAEN |
| | I622N- | ILFSNTIYKQFFEKELEQLPEAFALQLKSGGVLAQYGFDQLKFLLDVVKF |
| | N722R- | HLCRTTVPFAPGFKKVMKGGTGYQNSKQDESFYNLELTAYQLKDDFSEE |
| | A737S- | AWSARYFLLKIIYNYLFLPEFINNDKVFADTVAWLLGKNRQQAQSSKNK |
| | W765N- | NAYAFADIRLMESSESITDYMAYVQSQWNMEENKKEDDKKEDTRINFE |
| | S807R- | KFVLQLFVKGFDTFMKQSAYQFINTPQPQINPTDTNQQQADKLKALEKKI |
| | S819K- | CPFCHIQNNKIDPAKDEQIAFYVYCKLLDARHLSTLRNELIKYRASSESEL |
| | S827K- | FIHLTEIIELCLLHADNVPADYNQIYKNKEQCLQRLQPFIAENSDYTNWG |
| | N871K- | DLYVQTDDETPVVHRGIELSVKYGTAKLLEKLIAKDAKFKITENEFDLW |
| | D974Y- | HNARATIEAMIKKREEYHNLWLEAKEKDDKEKKTRIREKNNYARRFVE |
| | H1020R- | TKGEDYIDICDYIDRYNWLNNKLHFEHLKSLQNITIEMLGRMAGFVVLW |
| | I1064K | ERDFQYLDQQRVKQQGGALMDFSHGVPKYEELKPHYAYFKRLFLCNNY |
| | | SDTRNHIAHFNYLTKSAANYSLIDLINQLRELLRYDRKLKNAVAKTFIDL |
| | | FDKHGMVLKLKFDSQMHQLQVGSITSKKIFHLGTKKESDNIYTFQVPTA |
| | | YCNMCKQLLEMKN |
| 124 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | M311V- | FLNHKFHLPVKYQKEGQWVQFELSTCILQAVEHQNLDELQPYYDWSKW |
| | K318Q- | YINDKSGKLKKSITNNHIQIGDGESKRQKALLMWETEFTDKGELDLETY |
| | S352C- | HEKFIIDTLAASLRSVEKGELRDNGTPKETNSYHRNLKKVLQNHQALIFG |
| | V372L- | RRETPNQENRDNKQLAIYNLEVVKYLEHYFPIKSNVRKNSAADIVYYLK |
| | V501T- | SETIKSTVTHQLENAVRANLLRQGKYQHHDLKADATSENLTNLKRDEAF |
| | G548A- | VMNLITTCAFAANNIRNIIDPEQIKDILGKDDFIRSLKKDAFNKHIYGLFFP |
| | W621Q- | VGAEMIEPVSLWAMRGAVQQIRNNVAHYKKDALATIFNITKFEYPGAEN |
| | I622N- | ILFSNTIYKQFFEKELEQLPEAFALQLKSGGVLAQYGFDQLKFLLDVVKF |
| | N722R- | HLCRTTVPFAPGFKKVMKGGTGYQNSKQDESFYNLELTAYQLKDDFSEE |
| | A737S- | AWSARYFLLKIIYNYLFLPEFTNNDKVFADTVAWLLGKNRQQAQSSKNK |
| | A762G- | NAYAFADIRLMESSESITDYMAYVQSQQNMEENKKEDDKKEDTRINFEK |
| | W765N- | FVLQLFVKGFDTFMKQSAYQFINTPQPQINPTDTNQQQADKLKALEKKIC |
| | D800R- | PFCHIQNNKIDPAKDEQIAFYVYCKLLDARHLSTLRNELIKYRASSESELFI |
| | S807R- | HLTEIIELCLLHADNVPGDYNQIYKNKEQCLQRLQPFIAENSDYTNWGDL |
| | S819K- | YVQTDRETPVVHRGIELSVKYGTAKLLEKLIASDAKFKITENEFDLWHN |
| | N871K- | ARATIEAMIKKREEYHNLWLEAKEKDDKEKKTRIREKNNYARRFVETK |
| | D974Y- | GEDYIDICDYIDRYNWLNNKLHFEHLKSLQNITIEMLGRMAGFVVLWER |
| | I1064K | DFQYLDQQRVKQQGGALMDFSHGVPKYEELKPHYAYFKRLFLCNNYSD |
| | | TRNHIAHFNYLTKSAANYSLIDLINQLRELLHYDRKLKNAVAKTFIDLFD |
| | | KHGMVLKLKFDSQMHQLQVGSITSKKIFHLGTKKESDNIYTFQVPTAYC |
| | | NMCKQLLEMKN |
| 125 | H106Q- | MRVSKVKVTVNNKDTMVLMHRSAEKGALIYDTIDKKDRTEEIIPQKKQ |
| | E121N- | KSFELSIQNKTLLRYEAIKKQKLDRSKYKVVEDIFKYRKLPAKITLEDILP |
| | C125L- | FLNHKFQLPVKYQKEGQWVQFNLSTLILQAVEHQNLDELHPYYDWSKW |
| | M311V- | YINDKSGKLKKSITNNHIQIGDGESKRQKALLMWETDFTDKGELDLETY |
| | S547N- | HEKFIIDTLAASLRSVEKGELRDNGTPKETNSYHRNLKKVLQNHQALIFG |
| | Y559N- | RRETPNQENRDNKQLAIYNLEVVKYLEHYFPIKSNVRKNSAADIVYYLK |
| | Y858L- | SETIKSTVTHQLENAVRANLLRKGKYQHHDLKADATSENLTNLKRDEAF |
| | E915V- | VMNLITTSAFAANNIRNIIDPEQIKDIVGKDDFIRSLKKDAFNKHIYGLFFP |
| | S1062P | VGAEMIEPVSLWAMRGAVQQIRNNVAHYKKDALATIFNITKFEYPGAEN |
| | | ILFSNTIYKQFFEKELEQLPEAFALQLKSGGVLAQYGFDQLKFLLDVVKF |
| | | HLCRTVVPFAPGFKKVMKGGTGYQNSKQDESFYNLELTAYQLKDDFSE |
| | | EAWNGRYFLLKIIYNNLFLPEFTNNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRAASES |
| | | ELFIHLTEIIELCLLHADNVPADYWQIYKNKEQCLQRLQPFIAENSDYTN |
| | | WGDLYVQTDDETPVVHSGIELSVKYGTASLLEKLIASDAKFKITENEFDL |
| | | WHNARATIEAMIKKREELHNLWLEAKEKDDNEKKTRIREKNNYARRFV |
| | | ETKGEDYIDICDYIDRYNWLNNKLHFVHLKSLQNITIEMLGRMAGFVVL |
| | | WERDFQYLDQQRVKQQGGALMDFSHGVPKYEELKPHDAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITPKIIFHLGTKKESDNIYTFQVPT |
| | | AYCNMCKQLLEMKN |
| 126 | E121N- | MRVSKVKVTVNNKDTMVLMHRSAEKGALIYDTIDKKDRTEEIIPQKKQ |
| | M311V- | KSFELSIQNKTLLRYEAIKKQKLDRSKYKVVEDIFKYRKLPAKITLEDILP |
| | Q618E- | FLNHKFHLPVKYQKEGQWVQFNLSTCILQAVEHQNLDELHPYYDWSKW |
| | M656L- | YINDKSGKLKKSITNNHIQIGDGESKRQKALLMWETDFTDKGELDLETY |
| | Y858L | HEKFIIDTLAASLRSVEKGELRDNGTPKETNSYHRNLKKVLQNHQALIFG |
| | | RRETPNQENRDNKQLAIYNLEVVKYLEHYFPIKSNVRKNSAADIVYYLK |
| | | SETIKSTVTHQLENAVRANLLRKGKYQHHDLKADATSENLTNLKRDEAF |
| | | VMNLITTSAFAANNIRNIIDPEQIKDIVGKDDFIRSLKKDAFNKHIYGLFFP |
| | | VGAEMIEPVSLWAMRGAVQQIRNNVAHYKKDALATIFNITKFEYPGAEN |
| | | ILFSNTIYKQFFEKELEQLPEAFALQLKSGGVLAQYGFDQLKFLLDVVKF |
| | | HLCRTVVPFAPGFKKVMKGGTGYQNSKQDESFYNLELTAYQLKDDFSE |
| | | EAWSGRYFLLKIIYNYLFLPEFINNDKVFADTVAWLLGKNRQQAQSSKN |
| | | KNAYAFADIRLMESSESITDYMAYVESQWIMEENKKEDDKKEDTRINFE |
| | | KFVLQLFVKGFDTFLKQSAYQFINTPQPQINPTDTNQQQADKLKALEKKI |
| | | CPFCHIQNNKIDPAKDEQIAFYVYCKLLDANHLSTLRNELIKYRAASESEL |
| | | FIHLTEIIELCLLHADNVPADYWQIYKNKEQCLQRLQPFIAENSDYTNWG |
| | | DLYVQTDDETPVVHSGIELSVKYGTASLLEKLIASDAKFKITENEFDLWH |
| | | NARATIEAMIKKREELHNLWLEAKEKDDNEKKTRIREKNNYARRFVETK |
| | | GEDYIDICDYIDRYNWLNNKLHFEHLKSLQNITIEMLGRMAGFVVLWER |
| | | DFQYLDQQRVKQQGGALMDFSHGVPKYEELKPHDAYFKRLFLCNNYSD |
| | | TRNHIAHFNYLTKSAANYSLIDLINQLRELLHYDRKLKNAVAKTFIDLFD |
| | | KHGMVLKLKFDSQMHQLQVGSITSKIIFHLGTKKESDNIYTFQVPTAYCN |
| | | MCKQLLEMKN |
| 127 | E121N- | MRVSKVKVTVNNKDTMVLMHRSAEKGALIYDTIDKKDRTEEIIPQKKQ |
| | Y858L- | KSFELSIQNKTLLRYEAIKKQKLDRSKYKVVEDIFKYRKLPAKITLEDILP |
| | S1062P | FLNHKFHLPVKYQKEGQWVQFNLSTCILQAVEHQNLDELHPYYDWSKW |
| | | YINDKSGKLKKSITNNHIQIGDGESKRQKALLMWETDFTDKGELDLETY |
| | | HEKFIIDTLAASLRSVEKGELRDNGTPKETNSYHRNLKKVLQNHQALIFG |
| | | RRETPNQENRDNKQLAIYNLEVVKYLEHYFPIKSNVRKNSAADIVYYLK |
| | | SETIKSTVTHQLENAMRANLLRKGKYQHHDLKADATSENLTNLKRDEA |
| | | FVMNLITTSAFAANNIRNIIDPEQIKDIVGKDDFIRSLKKDAFNKHIYGLFF |
| | | PVGAEMIEPVSLWAMRGAVQQIRNNVAHYKKDALATIFNITKFEYPGAE |
| | | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSGRYFLLKIIYNYLFLPEFINNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRAASES |
| | | ELFIHLTEIIELCLLHADNVPADYWQIYKNKEQCLQRLQPFIAENSDYTN |
| | | WGDLYVQTDDETPVVHSGIELSVKYGTASLLEKLIASDAKFKITENEFDL |
| | | WHNARATIEAMIKKREELHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHDAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITPKIIFHLGTKKESDNIYTFQVPT |
| | | AYCNMCKQLLEMKN |
| 128 | E121N- | MRVSKVKVTVNNKDTMVLMHRSAEKGALIYDTIDKKDRTEEIIPQKKQ |
| | M311V- | KSFELSIQNKTLLRYEAIKKQKLDRSKYKVVEDIFKYRKLPAKITLEDILP |
| | S547N- | FLNHKFHLPVKYQKEGQWVQFNLSTCILQAVEHQNLDELHPYYDWSKW |
| | M656L- | YINDKSGKLKKSITNNHIQIGDGESKRQKALLMWETDFTDKGELDLETY |
| | M927I- | HEKFIIDTLAASLRSVEKGELRDNGTPKETNSYHRNLKKVLQNHQALIFG |
| | S1062P | RRETPNQENRDNKQLAIYNLEVVKYLEHYFPIKSNVRKNSAADIVYYLK |
| | | SETIKSTVTHQLENAVRANLLRKGKYQHHDLKADATSENLTNLKRDEAF |
| | | VMNLITTSAFAANNIRNIIDPEQIKDIVGKDDFIRSLKKDAFNKHIYGLFFP |
| | | VGAEMIEPVSLWAMRGAVQQIRNNVAHYKKDALATIFNITKFEYPGAEN |
| | | ILFSNTIYKQFFEKELEQLPEAFALQLKSGGVLAQYGFDQLKFLLDVVKF |
| | | HLCRTVVPFAPGFKKVMKGGTGYQNSKQDESFYNLELTAYQLKDDFSE |
| | | EAWNGRYFLLKIIYNYLFLPEFTNNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFLKQSAYQFINTPQPQINPTDTNQQQADKLKALEKK |
| | | ICPFCHIQNNKIDPAKDEQIAFYVYCKLLDANHLSTLRNELIKYRAASESE |
| | | LFIHLTEIIELCLLHADNVPADYWQIYKNKEQCLQRLQPFIAENSDYTNW |
| | | GDLYVQTDDETPVVHSGIELSVKYGTASLLEKLIASDAKFKITENEFDLW |
| | | HNARATIEAMIKKREEYHNLWLEAKEKDDNEKKTRIREKNNYARRFVE |
| | | TKGEDYIDICDYIDRYNWLNNKLHFEHLKSLQNITIEILGRMAGFVVLWE |
| | | RDFQYLDQQRVKQQGGALMDFSHGVPKYEELKPHDAYFKRLFLCNNYS |
| | | DTRNHIAHFNYLTKSAANYSLIDLINQLRELLHYDRKLKNAVAKTFIDLF |
| | | DKHGMVLKLKFDSQMHQLQVGSITPKIIFHLGTKKESDNIYTFQVPTAYC |
| | | NMCKQLLEMKN |
| 129 | H106Q- | MRVSKVKVTVNNKDTMVLMHRSAEKGALIYDTIDKKDRTEEIIPQKKQ |
| | E121N- | KSFELSIQNKTLLRYEAIKKQKLDRSKYKVVEDIFKYRKLPAKITLEDILP |
| | C125L- | FLNHKFQLPVKYQKEGQWVQFNLSTLILQAVEHQNLDELHPYYDWSKW |
| | M311V- | YINDKSGKLKKSITNNHIQIGDGESKRQKALLMWETDFTDKGELDLETY |
| | S547N- | HEKFIIDTLAASLRSVEKGELRDNGTPKETNSYHRNLKKVLQNHQALIFG |
| | Y559N- | RRETPNQENRDNKQLAIYNLEVVKYLEHYFPIKSNVRKNSAADIVYYLK |
| | Y858L- | SETIKSTVTHQLENAVRANLLRKGKYQHHDLKADATSENLTNLKRDEAF |
| | S1062P | VMNLITTSAFAANNIRNIIDPEQIKDIVGKDDFIRSLKKDAFNKHIYGLFFP |
| | | VGAEMIEPVSLWAMRGAVQQIRNNVAHYKKDALATIFNITKFEYPGAEN |
| | | ILFSNTIYKQFFEKELEQLPEAFALQLKSGGVLAQYGFDQLKFLLDVVKF |
| | | HLCRTVVPFAPGFKKVMKGGTGYQNSKQDESFYNLELTAYQLKDDFSE |
| | | EAWNGRYFLLKIIYNNLFLPEFINNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRAASES |
| | | ELFIHLTEIIELCLLHADNVPADYWQIYKNKEQCLQRLQPFIAENSDYTN |
| | | WGDLYVQTDDETPVVHSGIELSVKYGTASLLEKLIASDAKFKITENEFDL |
| | | WHNARATIEAMIKKREELHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHDAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITPKIIFHLGTKKESDNIYTFQVPT |
| | | AYCNMCKQLLEMKN |
| 130 | H106Q- | MRVSKVKVTVNNKDTMVLMHRSAEKGALIYDTIDKKDRTEEIIPQKKQ |
| | E121N- | KSFELSIQNKTLLRYEAIKKQKLDRSKYKVVEDIFKYRKLPAKITLEDILP |
| | M311V- | FLNHKFQLPVKYQKEGQWVQFNLSTCILQAVEHQNLDELHPYYDWSKW |
| | Q480Y- | YINDKSGKLKKSITNNHIQIGDGESKRQKALLMWETDFTDKGELDLETY |
| | S521A- | HEKFIIDTLAASLRSVEKGELRDNGTPKETNSYHRNLKKVLQNHQALIFG |
| | Q709H- | RRETPNQENRDNKQLAIYNLEVVKYLEHYFPIKSNVRKNSAADIVYYLK |
| | Y858L- | SETIKSTVTHQLENAVRANLLRKGKYQHHDLKADATSENLTNLKRDEAF |
| | E915V- | VMNLITTSAFAANNIRNIIDPEQIKDIVGKDDFIRSLKKDAFNKHIYGLFFP |
| | S1062P | VGAEMIEPVSLWAMRGAVQQIRNNVAHYKKDALATIFNITKFEYPGAEN |
| | | ILFSNTIYKQFFEKELEQLPEAFALQLKSGGVLAYYGFDQLKFLLDVVKF |
| | | HLCRTVVPFAPGFKKVMKGGTGYQNAKQDESFYNLELTAYQLKDDFSE |
| | | EAWSGRYFLLKIIYNYLFLPEFINNDKVFADTVAWLLGKNRQQAQSSKN |
| | | KNAYAFADIRLMESSESITDYMAYVQSQWIMEENKKEDDKKEDTRINFE |
| | | KFVLQLFVKGFDTFMKQSAYQFINTPQPQINPTDTNQQQADKLKALEKKI |
| | | CPFCHIQNNKIDPAKDEHIAFYVYCKLLDANHLSTLRNELIKYRAASESEL |
| | | FIHLTEIIELCLLHADNVPADYWQIYKNKEQCLQRLQPFIAENSDYTNWG |
| | | DLYVQTDDETPVVHSGIELSVKYGTASLLEKLIASDAKFKITENEFDLWH |
| | | NARATIEAMIKKREELHNLWLEAKEKDDNEKKTRIREKNNYARRFVETK |
| | | GEDYIDICDYIDRYNWLNNKLHFVHLKSLQNITIEMLGRMAGFVVLWER |
| | | DFQYLDQQRVKQQGGALMDFSHGVPKYEELKPHDAYFKRLFLCNNYSD |
| | | TRNHIAHFNYLTKSAANYSLIDLINQLRELLHYDRKLKNAVAKTFIDLFD |
| | | KHGMVLKLKFDSQMHQLQVGSITPKIIFHLGTKKESDNIYTFQVPTAYCN |
| | | MCKQLLEMKN |
| 131 | H106Q- | MRVSKVKVTVNNKDTMVLMHRSAEKGALIYDTIDKKDRTEEIIPQKKQ |
| | F200Y- | KSFELSIQNKTLLRYEAIKKQKLDRSKYKVVEDIFKYRKLPAKITLEDILP |
| | M311V- | FLNHKFQLPVKYQKEGQWVQFELSTCILQAVEHQNLDELHPYYDWSKW |
| | Q480Y- | YINDKSGKLKKSITNNHIQIGDGESKRQKALLMWETDFTDKGELDLETY |
| | S547N- | HEKYIIDTLAASLRSVEKGELRDNGTPKETNSYHRNLKKVLQNHQALIFG |
| | Y559N- | RRETPNQENRDNKQLAIYNLEVVKYLEHYFPIKSNVRKNSAADIVYYLK |
| | Y858L- | SETIKSTVTHQLENAVRANLLRKGKYQHHDLKADATSENLTNLKRDEAF |
| | M927I- | VMNLITTSAFAANNIRNIIDPEQIKDIVGKDDFIRSLKKDAFNKHIYGLFFP |
| | S1062P | VGAEMIEPVSLWAMRGAVQQIRNNVAHYKKDALATIFNITKFEYPGAEN |
| | | ILFSNTIYKQFFEKELEQLPEAFALQLKSGGVLAYYGFDQLKFLLDVVKF |
| | | HLCRTVVPFAPGFKKVMKGGTGYQNSKQDESFYNLELTAYQLKDDFSE |
| | | EAWNGRYFLLKIIYNNLFLPEFINNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRAASES |
| | | ELFIHLTEIIELCLLHADNVPADYWQIYKNKEQCLQRLQPFIAENSDYTN |
| | | WGDLYVQTDDETPVVHSGIELSVKYGTASLLEKLIASDAKFKITENEFDL |
| | | WHNARATIEAMIKKREELHNLWLEAKEKDDNEKKTRIREKNNYARRFV |
| | | ETKGEDYIDICDYIDRYNWLNNKLHFEHLKSLQNITIEILGRMAGFVVLW |
| | | ERDFQYLDQQRVKQQGGALMDFSHGVPKYEELKPHDAYFKRLFLCNNY |
| | | SDTRNHIAHFNYLTKSAANYSLIDLINQLRELLHYDRKLKNAVAKTFIDL |
| | | FDKHGMVLKLKFDSQMHQLQVGSITPKIIFHLGTKKESDNIYTFQVPTAY |
| | | CNMCKQLLEMKN |
| 132 | F200Y- | MRVSKVKVTVNNKDTMVLMHRSAEKGALIYDTIDKKDRTEEIIPQKKQ |
| | S547N- | KSFELSIQNKTLLRYEAIKKQKLDRSKYKVVEDIFKYRKLPAKITLEDILP |
| | L579V- | FLNHKFHLPVKYQKEGQWVQFELSTCILQAVEHQNLDELHPYYDWSKW |
| | M656L- | YINDKSGKLKKSITNNHIQIGDGESKRQKALLMWETDFTDKGELDLETY |
| | Y858L- | HEKYIIDTLAASLRSVEKGELRDNGTPKETNSYHRNLKKVLQNHQALIFG |
| | S1062P | RRETPNQENRDNKQLAIYNLEVVKYLEHYFPIKSNVRKNSAADIVYYLK |
| | | SETIKSTVTHQLENAMRANLLRKGKYQHHDLKADATSENLTNLKRDEA |
| | | FVMNLITTSAFAANNIRNIIDPEQIKDIVGKDDFIRSLKKDAFNKHIYGLFF |
| | | PVGAEMIEPVSLWAMRGAVQQIRNNVAHYKKDALATIFNITKFEYPGAE |
| | | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWNGRYFLLKIIYNYLFLPEFTNNDKVFADTVAWVLGKNRQQAQSS |
| | | KNKNAYAFADIRLMESSESITDYMAYVQSQWIMEENKKEDDKKEDTRIN |
| | | FEKFVLQLFVKGFDTFLKQSAYQFINTPQPQINPTDTNQQQADKLKALEK |
| | | KICPFCHIQNNKIDPAKDEQIAFYVYCKLLDANHLSTLRNELIKYRAASES |
| | | ELFIHLTEIIELCLLHADNVPADYWQIYKNKEQCLQRLQPFIAENSDYTN |
| | | WGDLYVQTDDETPVVHSGIELSVKYGTASLLEKLIASDAKFKITENEFDL |
| | | WHNARATIEAMIKKREELHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHDAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITPKIIFHLGTKKESDNIYTFQVPT |
| | | AYCNMCKQLLEMKN |
| 133 | H106Q- | MRVSKVKVTVNNKDTMVLMHRSAEKGALIYDTIDKKDRTEEIIPQKKQ |
| | F200Y- | KSFELSIQNKTLLRYEAIKKQKLDRSKYKVVEDIFKYRKLPAKITLEDILP |
| | S547N- | FLNHKFQLPVKYQKEGQWVQFELSTCILQAVEHQNLDELHPYYDWSKW |
| | M656L- | YINDKSGKLKKSITNNHIQIGDGESKRQKALLMWETDFTDKGELDLETY |
| | S1062P | HEKYIIDTLAASLRSVEKGELRDNGTPKETNSYHRNLKKVLQNHQALIFG |
| | | RRETPNQENRDNKQLAIYNLEVVKYLEHYFPIKSNVRKNSAADIVYYLK |
| | | SETIKSTVTHQLENAMRANLLRKGKYQHHDLKADATSENLTNLKRDEA |
| | | FVMNLITTSAFAANNIRNIIDPEQIKDIVGKDDFIRSLKKDAFNKHIYGLFF |
| | | PVGAEMIEPVSLWAMRGAVQQIRNNVAHYKKDALATIFNITKFEYPGAE |
| | | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWNGRYFLLKIIYNYLFLPEFTNNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFLKQSAYQFINTPQPQINPTDTNQQQADKLKALEKK |
| | | ICPFCHIQNNKIDPAKDEQIAFYVYCKLLDANHLSTLRNELIKYRAASESE |
| | | LFIHLTEIIELCLLHADNVPADYWQIYKNKEQCLQRLQPFIAENSDYTNW |
| | | GDLYVQTDDETPVVHSGIELSVKYGTASLLEKLIASDAKFKITENEFDLW |
| | | HNARATIEAMIKKREEYHNLWLEAKEKDDNEKKTRIREKNNYARRFVE |
| | | TKGEDYIDICDYIDRYNWLNNKLHFEHLKSLQNITIEMLGRMAGFVVLW |
| | | ERDFQYLDQQRVKQQGGALMDFSHGVPKYEELKPHDAYFKRLFLCNNY |
| | | SDTRNHIAHFNYLTKSAANYSLIDLINQLRELLHYDRKLKNAVAKTFIDL |
| | | FDKHGMVLKLKFDSQMHQLQVGSITPKIIFHLGTKKESDNIYTFQVPTAY |
| | | CNMCKQLLEMKN |
| 134 | F200Y- | MRVSKVKVTVNNKDTMVLMHRSAEKGALIYDTIDKKDRTEEIIPQKKQ |
| | M311V- | KSFELSIQNKTLLRYEAIKKQKLDRSKYKVVEDIFKYRKLPAKITLEDILP |
| | W546Y- | FLNHKFHLPVKYQKEGQWVQFELSTCILQAVEHQNLDELHPYYDWSKW |
| | Y559N- | YINDKSGKLKKSITNNHIQIGDGESKRQKALLMWETDFTDKGELDLETY |
| | M656L- | HEKYIIDTLAASLRSVEKGELRDNGTPKETNSYHRNLKKVLQNHQALIFG |
| | Y858L- | RRETPNQENRDNKQLAIYNLEVVKYLEHYFPIKSNVRKNSAADIVYYLK |
| | S1062P | SETIKSTVTHQLENAVRANLLRKGKYQHHDLKADATSENLTNLKRDEAF |
| | | VMNLITTSAFAANNIRNIIDPEQIKDIVGKDDFIRSLKKDAFNKHIYGLFFP |
| | | VGAEMIEPVSLWAMRGAVQQIRNNVAHYKKDALATIFNITKFEYPGAEN |
| | | ILFSNTIYKQFFEKELEQLPEAFALQLKSGGVLAQYGFDQLKFLLDVVKF |
| | | HLCRTVVPFAPGFKKVMKGGTGYQNSKQDESFYNLELTAYQLKDDFSE |
| | | EAYSGRYFLLKIIYNNLFLPEFTNNDKVFADTVAWLLGKNRQQAQSSKN |
| | | KNAYAFADIRLMESSESITDYMAYVQSQWIMEENKKEDDKKEDTRINFE |
| | | KFVLQLFVKGFDTFLKQSAYQFINTPQPQINPTDTNQQQADKLKALEKKI |
| | | CPFCHIQNNKIDPAKDEQIAFYVYCKLLDANHLSTLRNELIKYRAASESEL |
| | | FIHLTEIIELCLLHADNVPADYWQIYKNKEQCLQRLQPFIAENSDYTNWG |
| | | DLYVQTDDETPVVHSGIELSVKYGTASLLEKLIASDAKFKITENEFDLWH |
| | | NARATIEAMIKKREELHNLWLEAKEKDDNEKKTRIREKNNYARRFVETK |
| | | GEDYIDICDYIDRYNWLNNKLHFEHLKSLQNITIEMLGRMAGFVVLWER |
| | | DFQYLDQQRVKQQGGALMDFSHGVPKYEELKPHDAYFKRLFLCNNYSD |
| | | TRNHIAHFNYLTKSAANYSLIDLINQLRELLHYDRKLKNAVAKTFIDLFD |
| | | KHGMVLKLKFDSQMHQLQVGSITPKIIFHLGTKKESDNIYTFQVPTAYCN |
| | | MCKQLLEMKN |
| 135 | H139Q- | MRVSKVKVTVNNKDTMVLMHRSAEKGALIYDTIDKKDRTEEIIPQKKQ |
| | K318Q- | KSFELSIQNKTLLRYEAIKKQKLDRSKYKVVEDIFKYRKLPAKITLEDILP |
| | S352C- | FLNHKFHLPVKYQKEGQWVQFELSTCILQAVEHQNLDELQPYYDWSKW |
| | G548A- | YINDKSGKLKKSITNNHIQIGDGESKRQKALLMWETDFTDKGELDLETY |
| | N909D- | HEKFIIDTLAASLRSVEKGELRDNGTPKETNSYHRNLKKVLQNHQALIFG |
| | I1064K | RRETPNQENRDNKQLAIYNLEVVKYLEHYFPIKSNVRKNSAADIVYYLK |
| | | SETIKSTVTHQLENAMRANLLRQGKYQHHDLKADATSENLTNLKRDEA |
| | | FVMNLITTCAFAANNIRNIIDPEQIKDIVGKDDFIRSLKKDAFNKHIYGLFF |
| | | PVGAEMIEPVSLWAMRGAVQQIRNNVAHYKKDALATIFNITKFEYPGAE |
| | | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFINNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRAASES |
| | | ELFIHLTEIIELCLLHADNVPADYWQIYKNKEQCLQRLQPFIAENSDYTN |
| | | WGDLYVQTDDETPVVHSGIELSVKYGTASLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLDNKLHFEHLKSLQNITIEMLGRMAGFVVL |
| | | WERDFQYLDQQRVKQQGGALMDFSHGVPKYEELKPHDAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 136 | H139Q- | MRVSKVKVTVNNKDTMVLMHRSAEKGALIYDTIDKKDRTEEIIPQKKQ |
| | H164R- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | S352C- | YINDKSGKLKKSITNNRIQIGDGESKRQKALLMWETDFTDKGELDLETY |
| | V372L- | HEKFIIDTLAASLRSVEKGELRDNGTPKETNSYHRNLKKVLQNHQALIFG |
| | I1064K | RRETPNQENRDNKQLAIYNLEVVKYLEHYFPIKSNVRKNSAADIVYYLK |
| | | SETIKSTVTHQLENAMRANLLRQGKYQHHDLKADATSENLTNLKRDEA |
| | | FVMNLITTCAFAANNIRNIIDPEQIKDILGKDDFIRSLKKDAFNKHIYGLFF |
| | | PVGAEMIEPVSLWAMRGAVQQIRNNVAHYKKDALATIFNITKFEYPGAE |
| | | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSGRYFLLKIIYNYLFLPEFTNNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRAASES |
| | | ELFIHLTEIIELCLLHADNVPADYWQIYKNKEQCLQRLQPFIAENSDYTN |
| | | WGDLYVQTDDETPVVHSGIELSVKYGTASLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHDAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 137 | H139Q- | MRVSKVKVTVNNKDTMVLMHRSAEKGALIYDTIDKKDRTEEIIPQKKQ |
| | V372L- | KSFELSIQNKTLLRYEAIKKQKLDRSKYKVVEDIFKYRKLPAKITLEDILP |
| | G548A- | FLNHKFHLPVKYQKEGQWVQFELSTCILQAVEHQNLDELQPYYDWSKW |
| | T747L- | YINDKSGKLKKSITNNHIQIGDGESKRQKALLMWETDFTDKGELDLETY |
| | I1064K | HEKFIIDTLAASLRSVEKGELRDNGTPKETNSYHRNLKKVLQNHQALIFG |
| | | RRETPNQENRDNKQLAIYNLEVVKYLEHYFPIKSNVRKNSAADIVYYLK |
| | | SETIKSTVTHQLENAMRANLLRKGKYQHHDLKADATSENLTNLKRDEA |
| | | FVMNLITTSAFAANNIRNIIDPEQIKDILGKDDFIRSLKKDAFNKHIYGLFF |
| | | PVGAEMIEPVSLWAMRGAVQQIRNNVAHYKKDALATIFNITKFEYPGAE |
| | | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFINNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRAASES |
| | | ELFIHLLEIIELCLLHADNVPADYWQIYKNKEQCLQRLQPFIAENSDYTN |
| | | WGDLYVQTDDETPVVHSGIELSVKYGTASLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHDAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 138 | H139Q- | MRVSKVKVTVNNKDTMVLMHRSAEKGALIYDTIDKKDRTEEIIPQKKQ |
| | Q175K- | KSFELSIQNKTLLRYEAIKKQKLDRSKYKVVEDIFKYRKLPAKITLEDILP |
| | D184E- | FLNHKFHLPVKYQKEGQWVQFELSTCILQAVEHQNLDELQPYYDWSKW |
| | K318Q- | YINDKSGKLKKSITNNHIQIGDGESKRKKALLMWETEFTDKGELDLETY |
| | I555L- | HEKFIIDTLAASLRSVEKGELRDNGTPKETNSYHRNLKKVLQNHQALIFG |
| | I1064K- | RRETPNQENRDNKQLAIYNLEVVKYLEHYFPIKSNVRKNSAADIVYYLK |
| | A1085E | SETIKSTVTHQLENAMRANLLRQGKYQHHDLKADATSENLTNLKRDEA |
| | | FVMNLITTSAFAANNIRNIIDPEQIKDIVGKDDFIRSLKKDAFNKHIYGLFF |
| | | PVGAEMIEPVSLWAMRGAVQQIRNNVAHYKKDALATIFNITKFEYPGAE |
| | | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSGRYFLLKLIYNYLFLPEFINNDKVFADTVAWLLGKNRQQAQSS |
| | | KNKNAYAFADIRLMESSESITDYMAYVQSQWIMEENKKEDDKKEDTRIN |
| | | FEKFVLQLFVKGFDTFMKQSAYQFINTPQPQINPTDTNQQQADKLKALE |
| | | KKICPFCHIQNNKIDPAKDEQIAFYVYCKLLDANHLSTLRNELIKYRAASE |
| | | SELFIHLTEIIELCLLHADNVPADYWQIYKNKEQCLQRLQPFIAENSDYTN |
| | | WGDLYVQTDDETPVVHSGIELSVKYGTASLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHDAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TEYCNMCKQLLEMKN |
| 139 | H139Q- | MRVSKVKVTVNNKDTMVLMHRSAEKGALIYDTIDKKDRTEEIIPQKKQ |
| | Q175K- | KSFELSIQNKTLLRYEAIKKQKLDRSKYKVVEDIFKYRKLPAKITLEDILP |
| | D184E- | FLNHKFHLPVKYQKEGQWVQFELSTCILQAVEHQNLDELQPYYDWSKW |
| | K318Q- | YINDKSGKLKKSITNNHIQIGDGESKRKKALLMWETEFTDKGELDLETY |
| | L338I- | HEKFIIDTLAASLRSVEKGELRDNGTPKETNSYHRNLKKVLQNHQALIFG |
| | V372L- | RRETPNQENRDNKQLAIYNLEVVKYLEHYFPIKSNVRKNSAADIVYYLK |
| | G548A- | SETIKSTVTHQLENAMRANLLRQGKYQHHDLKADATSENLTNIKRDEAF |
| | T747L- | VMNLITTSAFAANNIRNIIDPEQIKDILGKDDFIRSLKKDAFNKHIYGLFFP |
| | N909D- | VGAEMIEPVSLWAMRGAVQQIRNNVAHYKKDALATIFNITKFEYPGAEN |
| | I1064K- | ILFSNTIYKQFFEKELEQLPEAFALQLKSGGVLAQYGFDQLKFLLDVVKF |
| | A1085E | HLCRTVVPFAPGFKKVMKGGTGYQNSKQDESFYNLELTAYQLKDDFSE |
| | | EAWSARYFLLKIIYNYLFLPEFINNDKVFADTVAWLLGKNRQQAQSSKN |
| | | KNAYAFADIRLMESSESITDYMAYVQSQWIMEENKKEDDKKEDTRINFE |
| | | KFVLQLFVKGFDTFMKQSAYQFINTPQPQINPTDTNQQQADKLKALEKKI |
| | | CPFCHIQNNKIDPAKDEQIAFYVYCKLLDANHLSTLRNELIKYRAASESEL |
| | | FIHLLEIIELCLLHADNVPADYWQIYKNKEQCLQRLQPFIAENSDYTNWG |
| | | DLYVQTDDETPVVHSGIELSVKYGTASLLEKLIASDAKFKITENEFDLWH |
| | | NARATIEAMIKKREEYHNLWLEAKEKDDNEKKTRIREKNNYARRFVET |
| | | KGEDYIDICDYIDRYNWLDNKLHFEHLKSLQNITIEMLGRMAGFVVLWE |
| | | RDFQYLDQQRVKQQGGALMDFSHGVPKYEELKPHDAYFKRLFLCNNYS |
| | | DTRNHIAHFNYLTKSAANYSLIDLINQLRELLHYDRKLKNAVAKTFIDLF |
| | | DKHGMVLKLKFDSQMHQLQVGSITSKKIFHLGTKKESDNIYTFQVPTEY |
| | | CNMCKQLLEMKN |
| 140 | H139Q- | MRVSKVKVTVNNKDTMVLMHRSAEKGALIYDTIDKKDRTEEIIPQKKQ |
| | L338I- | KSFELSIQNKTLLRYEAIKKQKLDRSKYKVVEDIFKYRKLPAKITLEDILP |
| | V372L- | FLNHKFHLPVKYQKEGQWVQFELSTCILQAVEHQNLDELQPYYDWSKW |
| | G517N- | YINDKSGKLKKSITNNHIQIGDGESKRQKALLMWETDFTDKGELDLETY |
| | A737S- | HEKFIIDTLAASLRSVEKGELRDNGTPKETNSYHRNLKKVLQNHQALIFG |
| | N909D- | RRETPNQENRDNKQLAIYNLEVVKYLEHYFPIKSNVRKNSAADIVYYLK |
| | I1064K | SETIKSTVTHQLENAMRANLLRKGKYQHHDLKADATSENLTNIKRDEAF |
| | | VMNLITTSAFAANNIRNIIDPEQIKDILGKDDFIRSLKKDAFNKHIYGLFFP |
| | | VGAEMIEPVSLWAMRGAVQQIRNNVAHYKKDALATIFNITKFEYPGAEN |
| | | ILFSNTIYKQFFEKELEQLPEAFALQLKSGGVLAQYGFDQLKFLLDVVKF |
| | | HLCRTVVPFAPGFKKVMKGGTNYQNSKQDESFYNLELTAYQLKDDFSE |
| | | EAWSGRYFLLKIIYNYLFLPEFINNDKVFADTVAWLLGKNRQQAQSSKN |
| | | KNAYAFADIRLMESSESITDYMAYVQSQWIMEENKKEDDKKEDTRINFE |
| | | KFVLQLFVKGFDTFMKQSAYQFINTPQPQINPTDTNQQQADKLKALEKKI |
| | | CPFCHIQNNKIDPAKDEQIAFYVYCKLLDANHLSTLRNELIKYRASSESEL |
| | | FIHLTEIIELCLLHADNVPADYWQIYKNKEQCLQRLQPFIAENSDYTNWG |
| | | DLYVQTDDETPVVHSGIELSVKYGTASLLEKLIASDAKFKITENEFDLWH |
| | | NARATIEAMIKKREEYHNLWLEAKEKDDNEKKTRIREKNNYARRFVET |
| | | KGEDYIDICDYIDRYNWLDNKLHFEHLKSLQNITIEMLGRMAGFVVLWE |
| | | RDFQYLDQQRVKQQGGALMDFSHGVPKYEELKPHDAYFKRLFLCNNYS |
| | | DTRNHIAHFNYLTKSAANYSLIDLINQLRELLHYDRKLKNAVAKTFIDLF |
| | | DKHGMVLKLKFDSQMHQLQVGSITSKKIFHLGTKKESDNIYTFQVPTAY |
| | | CNMCKQLLEMKN |
| 141 | H139Q- | MRVSKVKVTVNNKDTMVLMHRSAEKGALIYDTIDKKDRTEEIIPQKKQ |
| | K318Q- | KSFELSIQNKTLLRYEAIKKQKLDRSKYKVVEDIFKYRKLPAKITLEDILP |
| | L338I- | FLNHKFHLPVKYQKEGQWVQFELSTCILQAVEHQNLDELQPYYDWSKW |
| | V372L- | YINDKSGKLKKSITNNHIQIGDGESKRQKALLMWETDFTDKGELDLETY |
| | G548A- | HEKFIIDTLAASLRSVEKGELRDNGTPKETNSYHRNLKKVLQNHQALIFG |
| | T747L- | RRETPNQENRDNKQLAIYNLEVVKYLEHYFPIKSNVRKNSAADIVYYLK |
| | I1064K- | SETIKSTVTHQLENAMRANLLRQGKYQHHDLKADATSENLTNIKRDEAF |
| | A1085E | VMNLITTSAFAANNIRNIIDPEQIKDILGKDDFIRSLKKDAFNKHIYGLFFP |
| | | VGAEMIEPVSLWAMRGAVQQIRNNVAHYKKDALATIFNITKFEYPGAEN |
| | | ILFSNTIYKQFFEKELEQLPEAFALQLKSGGVLAQYGFDQLKFLLDVVKF |
| | | HLCRTVVPFAPGFKKVMKGGTGYQNSKQDESFYNLELTAYQLKDDFSE |
| | | EAWSARYFLLKIIYNYLFLPEFINNDKVFADTVAWLLGKNRQQAQSSKN |
| | | KNAYAFADIRLMESSESITDYMAYVQSQWIMEENKKEDDKKEDTRINFE |
| | | KFVLQLFVKGFDTFMKQSAYQFINTPQPQINPTDTNQQQADKLKALEKKI |
| | | CPFCHIQNNKIDPAKDEQIAFYVYCKLLDANHLSTLRNELIKYRAASESEL |
| | | FIHLLEIIELCLLHADNVPADYWQIYKNKEQCLQRLQPFIAENSDYTNWG |
| | | DLYVQTDDETPVVHSGIELSVKYGTASLLEKLIASDAKFKITENEFDLWH |
| | | NARATIEAMIKKREEYHNLWLEAKEKDDNEKKTRIREKNNYARRFVET |
| | | KGEDYIDICDYIDRYNWLNNKLHFEHLKSLQNITIEMLGRMAGFVVLWE |
| | | RDFQYLDQQRVKQQGGALMDFSHGVPKYEELKPHDAYFKRLFLCNNYS |
| | | DTRNHIAHFNYLTKSAANYSLIDLINQLRELLHYDRKLKNAVAKTFIDLF |
| | | DKHGMVLKLKFDSQMHQLQVGSITSKKIFHLGTKKESDNIYTFQVPTEY |
| | | CNMCKQLLEMKN |
| 142 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | G517N- | FLNHKFHLPVKYQKEGQWVQFELSTCILQAVEHQNLDELQPYYDWSKW |
| | G548A- | YINDKSGKLKKSITNNHIQIGDGESKRQKALLMWETEFTDKGELDLETY |
| | I1064K | HEKFIIDTLAASLRSVEKGELRDNGTPKETNSYHRNLKKVLQNHQALIFG |
| | | RRETPNQENRDNKQLAIYNLEVVKYLEHYFPIKSNVRKNSAADIVYYLK |
| | | SETIKSTVTHQLENAMRANLLRKGKYQHHDLKADATSENLTNLKRDEA |
| | | FVMNLITTSAFAANNIRNIIDPEQIKDIVGKDDFIRSLKKDAFNKHIYGLFF |
| | | PVGAEMIEPVSLWAMRGAVQQIRNNVAHYKKDALATIFNITKFEYPGAE |
| | | NILFSNTIYKQFFEKELEQLPEAFALQLKSGGVLAQYGFDQLKFLLDVVK |
| | | FHLCRTVVPFAPGFKKVMKGGTNYQNSKQDESFYNLELTAYQLKDDFS |
| | | EEAWSARYFLLKIIYNYLFLPEFINNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRAASES |
| | | ELFIHLTEIIELCLLHADNVPADYWQIYKNKEQCLQRLQPFIAENSDYTN |
| | | WGDLYVQTDDETPVVHSGIELSVKYGTASLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHDAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 143 | Q175K- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | S352C- | FLNHKFHLPVKYQKEGQWVQFELSTCILQAVEHQNLDELHPYYDWSKW |
| | V372L- | YINDKSGKLKKSITNNHIQIGDGESKRKKALLMWETEFTDKGELDLETY |
| | H496S- | HEKFIIDTLAASLRSVEKGELRDNGTPKETNSYHRNLKKVLQNHQALIFG |
| | G517N | RRETPNQENRDNKQLAIYNLEVVKYLEHYFPIKSNVRKNSAADIVYYLK |
| | | SETIKSTVTHQLENAMRANLLRKGKYQHHDLKADATSENLTNLKRDEA |
| | | FVMNLITTCAFAANNIRNIIDPEQIKDILGKDDFIRSLKKDAFNKHIYGLFF |
| | | PVGAEMIEPVSLWAMRGAVQQIRNNVAHYKKDALATIFNITKFEYPGAE |
| | | NILFSNTIYKQFFEKELEQLPEAFALQLKSGGVLAQYGFDQLKFLLDVVK |
| | | FSLCRTVVPFAPGFKKVMKGGTNYQNSKQDESFYNLELTAYQLKDDFSE |
| | | EAWSGRYFLLKIIYNYLFLPEFINNDKVFADTVAWLLGKNRQQAQSSKN |
| | | KNAYAFADIRLMESSESITDYMAYVQSQWIMEENKKEDDKKEDTRINFE |
| | | KFVLQLFVKGFDTFMKQSAYQFINTPQPQINPTDTNQQQADKLKALEKKI |
| | | CPFCHIQNNKIDPAKDEQIAFYVYCKLLDANHLSTLRNELIKYRAASESEL |
| | | FIHLTEIIELCLLHADNVPADYWQIYKNKEQCLQRLQPFIAENSDYTNWG |
| | | DLYVQTDDETPVVHSGIELSVKYGTASLLEKLIASDAKFKITENEFDLWH |
| | | NARATIEAMIKKREEYHNLWLEAKEKDDNEKKTRIREKNNYARRFVET |
| | | KGEDYIDICDYIDRYNWLNNKLHFEHLKSLQNITIEMLGRMAGFVVLWE |
| | | RDFQYLDQQRVKQQGGALMDFSHGVPKYEELKPHDAYFKRLFLCNNYS |
| | | DTRNHIAHFNYLTKSAANYSLIDLINQLRELLHYDRKLKNAVAKTFIDLF |
| | | DKHGMVLKLKFDSQMHQLQVGSITSKIIFHLGTKKESDNIYTFQVPTAYC |
| | | NMCKQLLEMKN |
| 144 | H139Q- | MRVSKVKVTVNNKDTMVLMHRSAEKGALIYDTIDKKDRTEEIIPQKKQ |
| | V372L- | KSFELSIQNKTLLRYEAIKKQKLDRSKYKVVEDIFKYRKLPAKITLEDILP |
| | L381M- | FLNHKFHLPVKYQKEGQWVQFELSTCILQAVEHQNLDELQPYYDWSKW |
| | G517N- | YINDKSGKLKKSITNNHIQIGDGESKRQKALLMWETDFTDKGELDLETY |
| | A737S- | HEKFIIDTLAASLRSVEKGELRDNGTPKETNSYHRNLKKVLQNHQALIFG |
| | I1064K | RRETPNQENRDNKQLAIYNLEVVKYLEHYFPIKSNVRKNSAADIVYYLK |
| | | SETIKSTVTHQLENAMRANLLRKGKYQHHDLKADATSENLTNLKRDEA |
| | | FVMNLITTSAFAANNIRNIIDPEQIKDILGKDDFIRSMKKDAFNKHIYGLFF |
| | | PVGAEMIEPVSLWAMRGAVQQIRNNVAHYKKDALATIFNITKFEYPGAE |
| | | NILFSNTIYKQFFEKELEQLPEAFALQLKSGGVLAQYGFDQLKFLLDVVK |
| | | FHLCRTVVPFAPGFKKVMKGGTNYQNSKQDESFYNLELTAYQLKDDFS |
| | | EEAWSGRYFLLKIIYNYLFLPEFINNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDDETPVVHSGIELSVKYGTASLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHDAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 145 | H139Q- | MRVSKVKVTVNNKDTMVLMHRSAEKGALIYDTIDKKDRTEEIIPQKKQ |
| | Q175K- | KSFELSIQNKTLLRYEAIKKQKLDRSKYKVVEDIFKYRKLPAKITLEDILP |
| | V372L- | FLNHKFHLPVKYQKEGQWVQFELSTCILQAVEHQNLDELQPYYDWSKW |
| | N909D- | YINDKSGKLKKSITNNHIQIGDGESKRKKALLMWETDFTDKGELDLETY |
| | I1064K | HEKFIIDTLAASLRSVEKGELRDNGTPKETNSYHRNLKKVLQNHQALIFG |
| | | RRETPNQENRDNKQLAIYNLEVVKYLEHYFPIKSNVRKNSAADIVYYLK |
| | | SETIKSTVTHQLENAMRANLLRKGKYQHHDLKADATSENLTNLKRDEA |
| | | FVMNLITTSAFAANNIRNIIDPEQIKDILGKDDFIRSLKKDAFNKHIYGLFF |
| | | PVGAEMIEPVSLWAMRGAVQQIRNNVAHYKKDALATIFNITKFEYPGAE |
| | | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSGRYFLLKIIYNYLFLPEFINNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRAASES |
| | | ELFIHLTEIIELCLLHADNVPADYWQIYKNKEQCLQRLQPFIAENSDYTN |
| | | WGDLYVQTDDETPVVHSGIELSVKYGTASLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLDNKLHFEHLKSLQNITIEMLGRMAGFVVL |
| | | WERDFQYLDQQRVKQQGGALMDFSHGVPKYEELKPHDAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 146 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | G548A- | HEKFIIDTLAASLRSVEKGELRDNGTPKETNSYHRNLKKVLQNHQALIFG |
| | A737S- | RRETPNQENRDNKQLAIYNLEVVKYLEHYFPIKSNVRKNSAADIVYYLK |
| | S827R- | SETIKSTVTHQLENAMRANLLRQGKYQHHDLKADATSENLTNLKRDEA |
| | I1064K | FVMNLITTSAFAANNIRNIIDPEQIKDILGKDDFIRSLKKDAFNKHIYGLFF |
| | | PVGAEMIEPVSLWAMRGAVQQIRNNVAHYKKDALATIFNITKFEYPGAE |
| | | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFINNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDDETPVVHSGIELSVKYGTASLLEKLIARDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHDAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 147 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | G548A- | HEKFIIDTLAASLRSVEKGELRDNGTPKETNSYHRNLKKVLQNHQALIFG |
| | D569H- | RRETPNQENRDNKQLAIYNLEVVKYLEHYFPIKSNVRKNSAADIVYYLK |
| | A737S- | SETIKSTVTHQLENAMRANLLRQGKYQHHDLKADATSENLTNLKRDEA |
| | I1064K | FVMNLITTSAFAANNIRNIIDPEQIKDILGKDDFIRSLKKDAFNKHIYGLFF |
| | | PVGAEMIEPVSLWAMRGAVQQIRNNVAHYKKDALATIFNITKFEYPGAE |
| | | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFTNNHKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDDETPVVHSGIELSVKYGTASLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHDAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 148 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | G548A- | HEKFIIDTLAASLRSVEKGELRDNGTPKETNSYHRNLKKVLQNHQALIFG |
| | Q585L- | RRETPNQENRDNKQLAIYNLEVVKYLEHYFPIKSNVRKNSAADIVYYLK |
| | A737S- | SETIKSTVTHQLENAMRANLLRQGKYQHHDLKADATSENLTNLKRDEA |
| | I1064K | FVMNLITTSAFAANNIRNIIDPEQIKDILGKDDFIRSLKKDAFNKHIYGLFF |
| | | PVGAEMIEPVSLWAMRGAVQQIRNNVAHYKKDALATIFNITKFEYPGAE |
| | | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFTNNDKVFADTVAWLLGKNRLQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDDETPVVHSGIELSVKYGTASLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHDAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 149 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | G548A- | HEKFIIDTLAASLRSVEKGELRDNGTPKETNSYHRNLKKVLQNHQALIFG |
| | A737S- | RRETPNQENRDNKQLAIYNLEVVKYLEHYFPIKSNVRKNSAADIVYYLK |
| | A846P- | SETIKSTVTHQLENAMRANLLRQGKYQHHDLKADATSENLTNLKRDEA |
| | I1064K | FVMNLITTSAFAANNIRNIIDPEQIKDILGKDDFIRSLKKDAFNKHIYGLFF |
| | | PVGAEMIEPVSLWAMRGAVQQIRNNVAHYKKDALATIFNITKFEYPGAE |
| | | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFINNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDDETPVVHSGIELSVKYGTASLLEKLIASDAKFKITENEFDL |
| | | WHNARPTIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHDAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 150 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | G548A- | HEKFIIDTLAASLRSVEKGELRDNGTPKETNSYHRNLKKVLQNHQALIFG |
| | A737S- | RRETPNQENRDNKQLAIYNLEVVKYLEHYFPIKSNVRKNSAADIVYYLK |
| | T789Q- | SETIKSTVTHQLENAMRANLLRQGKYQHHDLKADATSENLTNLKRDEA |
| | I1064K | FVMNLITTSAFAANNIRNIIDPEQIKDILGKDDFIRSLKKDAFNKHIYGLFF |
| | | PVGAEMIEPVSLWAMRGAVQQIRNNVAHYKKDALATIFNITKFEYPGAE |
| | | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFINNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYQN |
| | | WGDLYVQTDDETPVVHSGIELSVKYGTASLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHDAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 151 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | G548A- | HEKFIIDTLAASLRSVEKGELRDNGTPKETNSYHRNLKKVLQNHQALIFG |
| | N722R- | RRETPNQENRDNKQLAIYNLEVVKYLEHYFPIKSNVRKNSAADIVYYLK |
| | A737S- | SETIKSTVTHQLENAMRANLLRQGKYQHHDLKADATSENLTNLKRDEA |
| | I1064K | FVMNLITTSAFAANNIRNIIDPEQIKDILGKDDFIRSLKKDAFNKHIYGLFF |
| | | PVGAEMIEPVSLWAMRGAVQQIRNNVAHYKKDALATIFNITKFEYPGAE |
| | | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFTNNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDARHLSTLRNELIKYRASSES |
| | | ELFIHLTEIIELCLLHADNVPADYWQIYKNKEQCLQRLQPFIAENSDYTN |
| | | WGDLYVQTDDETPVVHSGIELSVKYGTASLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHDAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 152 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | G548A- | HEKFIIDTLAASLRSVEKGELRDNGTPKETNSYHRNLKKVLQNHQALIFG |
| | N567S- | RRETPNQENRDNKQLAIYNLEVVKYLEHYFPIKSNVRKNSAADIVYYLK |
| | A737S- | SETIKSTVTHQLENAMRANLLRQGKYQHHDLKADATSENLTNLKRDEA |
| | I1064K | FVMNLITTSAFAANNIRNIIDPEQIKDILGKDDFIRSLKKDAFNKHIYGLFF |
| | | PVGAEMIEPVSLWAMRGAVQQIRNNVAHYKKDALATIFNITKFEYPGAE |
| | | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFTSNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDDETPVVHSGIELSVKYGTASLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHDAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 153 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | G548A- | HEKFIIDTLAASLRSVEKGELRDNGTPKETNSYHRNLKKVLQNHQALIFG |
| | A737S- | RRETPNQENRDNKQLAIYNLEVVKYLEHYFPIKSNVRKNSAADIVYYLK |
| | D800K- | SETIKSTVTHQLENAMRANLLRQGKYQHHDLKADATSENLTNLKRDEA |
| | I1064K | FVMNLITTSAFAANNIRNIIDPEQIKDILGKDDFIRSLKKDAFNKHIYGLFF |
| | | PVGAEMIEPVSLWAMRGAVQQIRNNVAHYKKDALATIFNITKFEYPGAE |
| | | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFTNNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDKETPVVHSGIELSVKYGTASLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHDAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 154 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | G548A- | HEKFIIDTLAASLRSVEKGELRDNGTPKETNSYHRNLKKVLQNHQALIFG |
| | A737S- | RRETPNQENRDNKQLAIYNLEVVKYLEHYFPIKSNVRKNSAADIVYYLK |
| | S919Q- | SETIKSTVTHQLENAMRANLLRQGKYQHHDLKADATSENLTNLKRDEA |
| | I1064K | FVMNLITTSAFAANNIRNIIDPEQIKDILGKDDFIRSLKKDAFNKHIYGLFF |
| | | PVGAEMIEPVSLWAMRGAVQQIRNNVAHYKKDALATIFNITKFEYPGAE |
| | | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFINNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDDETPVVHSGIELSVKYGTASLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKQLQNITIEMLGRMAGFVVL |
| | | WERDFQYLDQQRVKQQGGALMDFSHGVPKYEELKPHDAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 155 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | G548A- | HEKFIIDTLAASLRSVEKGELRDNGTPKETNSYHRNLKKVLQNHQALIFG |
| | A737S- | RRETPNQENRDNKQLAIYNLEVVKYLEHYFPIKSNVRKNSAADIVYYLK |
| | N790K- | SETIKSTVTHQLENAMRANLLRQGKYQHHDLKADATSENLTNLKRDEA |
| | I1064K | FVMNLITTSAFAANNIRNIIDPEQIKDILGKDDFIRSLKKDAFNKHIYGLFF |
| | | PVGAEMIEPVSLWAMRGAVQQIRNNVAHYKKDALATIFNITKFEYPGAE |
| | | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFINNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTK |
| | | WGDLYVQTDDETPVVHSGIELSVKYGTASLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHDAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 156 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | V501T- | HEKFIIDTLAASLRSVEKGELRDNGTPKETNSYHRNLKKVLQNHQALIFG |
| | G548A- | RRETPNQENRDNKQLAIYNLEVVKYLEHYFPIKSNVRKNSAADIVYYLK |
| | A737S- | SETIKSTVTHQLENAMRANLLRQGKYQHHDLKADATSENLTNLKRDEA |
| | I1064K | FVMNLITTSAFAANNIRNIIDPEQIKDILGKDDFIRSLKKDAFNKHIYGLFF |
| | | PVGAEMIEPVSLWAMRGAVQQIRNNVAHYKKDALATIFNITKFEYPGAE |
| | | NILFSNTIYKQFFEKELEQLPEAFALQLKSGGVLAQYGFDQLKFLLDVVK |
| | | FHLCRTTVPFAPGFKKVMKGGTGYQNSKQDESFYNLELTAYQLKDDFSE |
| | | EAWSARYFLLKIIYNYLFLPEFINNDKVFADTVAWLLGKNRQQAQSSKN |
| | | KNAYAFADIRLMESSESITDYMAYVQSQWIMEENKKEDDKKEDTRINFE |
| | | KFVLQLFVKGFDTFMKQSAYQFINTPQPQINPTDTNQQQADKLKALEKKI |
| | | CPFCHIQNNKIDPAKDEQIAFYVYCKLLDANHLSTLRNELIKYRASSESEL |
| | | FIHLTEIIELCLLHADNVPADYWQIYKNKEQCLQRLQPFIAENSDYTNWG |
| | | DLYVQTDDETPVVHSGIELSVKYGTASLLEKLIASDAKFKITENEFDLWH |
| | | NARATIEAMIKKREEYHNLWLEAKEKDDNEKKTRIREKNNYARRFVET |
| | | KGEDYIDICDYIDRYNWLNNKLHFEHLKSLQNITIEMLGRMAGFVVLWE |
| | | RDFQYLDQQRVKQQGGALMDFSHGVPKYEELKPHDAYFKRLFLCNNYS |
| | | DTRNHIAHFNYLTKSAANYSLIDLINQLRELLHYDRKLKNAVAKTFIDLF |
| | | DKHGMVLKLKFDSQMHQLQVGSITSKKIFHLGTKKESDNIYTFQVPTAY |
| | | CNMCKQLLEMKN |
| 157 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | G548A- | HEKFIIDTLAASLRSVEKGELRDNGTPKETNSYHRNLKKVLQNHQALIFG |
| | A737S- | RRETPNQENRDNKQLAIYNLEVVKYLEHYFPIKSNVRKNSAADIVYYLK |
| | E801M- | SETIKSTVTHQLENAMRANLLRQGKYQHHDLKADATSENLTNLKRDEA |
| | I1064K | FVMNLITTSAFAANNIRNIIDPEQIKDILGKDDFIRSLKKDAFNKHIYGLFF |
| | | PVGAEMIEPVSLWAMRGAVQQIRNNVAHYKKDALATIFNITKFEYPGAE |
| | | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFINNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDDMTPVVHSGIELSVKYGTASLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHDAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 158 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | G548A- | HEKFIIDTLAASLRSVEKGELRDNGTPKETNSYHRNLKKVLQNHQALIFG |
| | A737S- | RRETPNQENRDNKQLAIYNLEVVKYLEHYFPIKSNVRKNSAADIVYYLK |
| | T847K- | SETIKSTVTHQLENAMRANLLRQGKYQHHDLKADATSENLTNLKRDEA |
| | I1064K | FVMNLITTSAFAANNIRNIIDPEQIKDILGKDDFIRSLKKDAFNKHIYGLFF |
| | | PVGAEMIEPVSLWAMRGAVQQIRNNVAHYKKDALATIFNITKFEYPGAE |
| | | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFINNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDDETPVVHSGIELSVKYGTASLLEKLIASDAKFKITENEFDL |
| | | WHNARAKIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHDAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 159 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | G548A- | HEKFIIDTLAASLRSVEKGELRDNGTPKETNSYHRNLKKVLQNHQALIFG |
| | A737S- | RRETPNQENRDNKQLAIYNLEVVKYLEHYFPIKSNVRKNSAADIVYYLK |
| | D870E- | SETIKSTVTHQLENAMRANLLRQGKYQHHDLKADATSENLTNLKRDEA |
| | I1064K | FVMNLITTSAFAANNIRNIIDPEQIKDILGKDDFIRSLKKDAFNKHIYGLFF |
| | | PVGAEMIEPVSLWAMRGAVQQIRNNVAHYKKDALATIFNITKFEYPGAE |
| | | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFINNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDDETPVVHSGIELSVKYGTASLLEKLIASDAKFKITENEFDL |
| | | WHNARATIEAMIKKREEYHNLWLEAKEKDENEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHDAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 160 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | G548A- | HEKFIIDTLAASLRSVEKGELRDNGTPKETNSYHRNLKKVLQNHQALIFG |
| | A737S- | RRETPNQENRDNKQLAIYNLEVVKYLEHYFPIKSNVRKNSAADIVYYLK |
| | W765R- | SETIKSTVTHQLENAMRANLLRQGKYQHHDLKADATSENLTNLKRDEA |
| | I1064K | FVMNLITTSAFAANNIRNIIDPEQIKDILGKDDFIRSLKKDAFNKHIYGLFF |
| | | PVGAEMIEPVSLWAMRGAVQQIRNNVAHYKKDALATIFNITKFEYPGAE |
| | | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFTNNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYRQIYKNKEQCLQRLQPFIAENSDYTNW |
| | | GDLYVQTDDETPVVHSGIELSVKYGTASLLEKLIASDAKFKITENEFDLW |
| | | HNARATIEAMIKKREEYHNLWLEAKEKDDNEKKTRIREKNNYARRFVE |
| | | TKGEDYIDICDYIDRYNWLNNKLHFEHLKSLQNITIEMLGRMAGFVVLW |
| | | ERDFQYLDQQRVKQQGGALMDFSHGVPKYEELKPHDAYFKRLFLCNNY |
| | | SDTRNHIAHFNYLTKSAANYSLIDLINQLRELLHYDRKLKNAVAKTFIDL |
| | | FDKHGMVLKLKFDSQMHQLQVGSITSKKIFHLGTKKESDNIYTFQVPTA |
| | | YCNMCKQLLEMKN |
| 161 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | G548A- | HEKFIIDTLAASLRSVEKGELRDNGTPKETNSYHRNLKKVLQNHQALIFG |
| | A737S- | RRETPNQENRDNKQLAIYNLEVVKYLEHYFPIKSNVRKNSAADIVYYLK |
| | D800G- | SETIKSTVTHQLENAMRANLLRQGKYQHHDLKADATSENLTNLKRDEA |
| | I1064K | FVMNLITTSAFAANNIRNIIDPEQIKDILGKDDFIRSLKKDAFNKHIYGLFF |
| | | PVGAEMIEPVSLWAMRGAVQQIRNNVAHYKKDALATIFNITKFEYPGAE |
| | | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFINNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDGETPVVHSGIELSVKYGTASLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHDAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 162 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | G548A- | HEKFIIDTLAASLRSVEKGELRDNGTPKETNSYHRNLKKVLQNHQALIFG |
| | A737S- | RRETPNQENRDNKQLAIYNLEVVKYLEHYFPIKSNVRKNSAADIVYYLK |
| | W765N- | SETIKSTVTHQLENAMRANLLRQGKYQHHDLKADATSENLTNLKRDEA |
| | I1064K | FVMNLITTSAFAANNIRNIIDPEQIKDILGKDDFIRSLKKDAFNKHIYGLFF |
| | | PVGAEMIEPVSLWAMRGAVQQIRNNVAHYKKDALATIFNITKFEYPGAE |
| | | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFTNNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYNQIYKNKEQCLQRLQPFIAENSDYTNW |
| | | GDLYVQTDDETPVVHSGIELSVKYGTASLLEKLIASDAKFKITENEFDLW |
| | | HNARATIEAMIKKREEYHNLWLEAKEKDDNEKKTRIREKNNYARRFVE |
| | | TKGEDYIDICDYIDRYNWLNNKLHFEHLKSLQNITIEMLGRMAGFVVLW |
| | | ERDFQYLDQQRVKQQGGALMDFSHGVPKYEELKPHDAYFKRLFLCNNY |
| | | SDTRNHIAHFNYLTKSAANYSLIDLINQLRELLHYDRKLKNAVAKTFIDL |
| | | FDKHGMVLKLKFDSQMHQLQVGSITSKKIFHLGTKKESDNIYTFQVPTA |
| | | YCNMCKQLLEMKN |
| 163 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | G548A- | HEKFIIDTLAASLRSVEKGELRDNGTPKETNSYHRNLKKVLQNHQALIFG |
| | A737S- | RRETPNQENRDNKQLAIYNLEVVKYLEHYFPIKSNVRKNSAADIVYYLK |
| | E864Q- | SETIKSTVTHQLENAMRANLLRQGKYQHHDLKADATSENLTNLKRDEA |
| | I1064K | FVMNLITTSAFAANNIRNIIDPEQIKDILGKDDFIRSLKKDAFNKHIYGLFF |
| | | PVGAEMIEPVSLWAMRGAVQQIRNNVAHYKKDALATIFNITKFEYPGAE |
| | | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFINNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDDETPVVHSGIELSVKYGTASLLEKLIASDAKFKITENEFDL |
| | | WHNARATIEAMIKKREEYHNLWLQAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHDAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 164 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | V492Q- | HEKFIIDTLAASLRSVEKGELRDNGTPKETNSYHRNLKKVLQNHQALIFG |
| | G548A- | RRETPNQENRDNKQLAIYNLEVVKYLEHYFPIKSNVRKNSAADIVYYLK |
| | A737S- | SETIKSTVTHQLENAMRANLLRQGKYQHHDLKADATSENLTNLKRDEA |
| | I1064K | FVMNLITTSAFAANNIRNIIDPEQIKDILGKDDFIRSLKKDAFNKHIYGLFF |
| | | PVGAEMIEPVSLWAMRGAVQQIRNNVAHYKKDALATIFNITKFEYPGAE |
| | | NILFSNTIYKQFFEKELEQLPEAFALQLKSGGVLAQYGFDQLKFLLDQVK |
| | | FHLCRTVVPFAPGFKKVMKGGTGYQNSKQDESFYNLELTAYQLKDDFS |
| | | EEAWSARYFLLKIIYNYLFLPEFINNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDDETPVVHSGIELSVKYGTASLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHDAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 165 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | G548A- | HEKFIIDTLAASLRSVEKGELRDNGTPKETNSYHRNLKKVLQNHQALIFG |
| | A737S- | RRETPNQENRDNKQLAIYNLEVVKYLEHYFPIKSNVRKNSAADIVYYLK |
| | N836P- | SETIKSTVTHQLENAMRANLLRQGKYQHHDLKADATSENLTNLKRDEA |
| | I1064K | FVMNLITTSAFAANNIRNIIDPEQIKDILGKDDFIRSLKKDAFNKHIYGLFF |
| | | PVGAEMIEPVSLWAMRGAVQQIRNNVAHYKKDALATIFNITKFEYPGAE |
| | | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFINNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDDETPVVHSGIELSVKYGTASLLEKLIASDAKFKITEPEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHDAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 166 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | G548A- | HEKFIIDTLAASLRSVEKGELRDNGTPKETNSYHRNLKKVLQNHQALIFG |
| | A737S- | RRETPNQENRDNKQLAIYNLEVVKYLEHYFPIKSNVRKNSAADIVYYLK |
| | S807T- | SETIKSTVTHQLENAMRANLLRQGKYQHHDLKADATSENLTNLKRDEA |
| | I1064K | FVMNLITTSAFAANNIRNIIDPEQIKDILGKDDFIRSLKKDAFNKHIYGLFF |
| | | PVGAEMIEPVSLWAMRGAVQQIRNNVAHYKKDALATIFNITKFEYPGAE |
| | | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFINNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDDETPVVHTGIELSVKYGTASLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHDAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 167 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | L537R- | HEKFIIDTLAASLRSVEKGELRDNGTPKETNSYHRNLKKVLQNHQALIFG |
| | G548A- | RRETPNQENRDNKQLAIYNLEVVKYLEHYFPIKSNVRKNSAADIVYYLK |
| | A737S- | SETIKSTVTHQLENAMRANLLRQGKYQHHDLKADATSENLTNLKRDEA |
| | I1064K | FVMNLITTSAFAANNIRNIIDPEQIKDILGKDDFIRSLKKDAFNKHIYGLFF |
| | | PVGAEMIEPVSLWAMRGAVQQIRNNVAHYKKDALATIFNITKFEYPGAE |
| | | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQRKDDFS |
| | | EEAWSARYFLLKIIYNYLFLPEFINNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDDETPVVHSGIELSVKYGTASLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHDAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 168 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | G548A- | HEKFIIDTLAASLRSVEKGELRDNGTPKETNSYHRNLKKVLQNHQALIFG |
| | A737S- | RRETPNQENRDNKQLAIYNLEVVKYLEHYFPIKSNVRKNSAADIVYYLK |
| | S807R- | SETIKSTVTHQLENAMRANLLRQGKYQHHDLKADATSENLTNLKRDEA |
| | N871K- | FVMNLITTSAFAANNIRNIIDPEQIKDILGKDDFIRSLKKDAFNKHIYGLFF |
| | I1064K | PVGAEMIEPVSLWAMRGAVQQIRNNVAHYKKDALATIFNITKFEYPGAE |
| | | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFINNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDDETPVVHRGIELSVKYGTASLLEKLIASDAKFKITENEFDL |
| | | WHNARATIEAMIKKREEYHNLWLEAKEKDDKEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHDAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 169 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | G548A- | HEKFIIDTLAASLRSVEKGELRDNGTPKETNSYHRNLKKVLQNHQALIFG |
| | A737S- | RRETPNQENRDNKQLAIYNLEVVKYLEHYFPIKSNVRKNSAADIVYYLK |
| | E867A- | SETIKSTVTHQLENAMRANLLRQGKYQHHDLKADATSENLTNLKRDEA |
| | I1064K | FVMNLITTSAFAANNIRNIIDPEQIKDILGKDDFIRSLKKDAFNKHIYGLFF |
| | | PVGAEMIEPVSLWAMRGAVQQIRNNVAHYKKDALATIFNITKFEYPGAE |
| | | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFINNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDDETPVVHSGIELSVKYGTASLLEKLIASDAKFKITENEFDL |
| | | WHNARATIEAMIKKREEYHNLWLEAKAKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHDAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 170 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | G548A- | HEKFIIDTLAASLRSVEKGELRDNGTPKETNSYHRNLKKVLQNHQALIFG |
| | A737S- | RRETPNQENRDNKQLAIYNLEVVKYLEHYFPIKSNVRKNSAADIVYYLK |
| | E801G- | SETIKSTVTHQLENAMRANLLRQGKYQHHDLKADATSENLTNLKRDEA |
| | I1064K | FVMNLITTSAFAANNIRNIIDPEQIKDILGKDDFIRSLKKDAFNKHIYGLFF |
| | | PVGAEMIEPVSLWAMRGAVQQIRNNVAHYKKDALATIFNITKFEYPGAE |
| | | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFINNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDDGTPVVHSGIELSVKYGTASLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHDAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 171 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | G548A- | HEKFIIDTLAASLRSVEKGELRDNGTPKETNSYHRNLKKVLQNHQALIFG |
| | A737S- | RRETPNQENRDNKQLAIYNLEVVKYLEHYFPIKSNVRKNSAADIVYYLK |
| | Q766M- | SETIKSTVTHQLENAMRANLLRQGKYQHHDLKADATSENLTNLKRDEA |
| | I1064K | FVMNLITTSAFAANNIRNIIDPEQIKDILGKDDFIRSLKKDAFNKHIYGLFF |
| | | PVGAEMIEPVSLWAMRGAVQQIRNNVAHYKKDALATIFNITKFEYPGAE |
| | | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFINNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWMIYKNKEQCLQRLQPFIAENSDYTN |
| | | WGDLYVQTDDETPVVHSGIELSVKYGTASLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHDAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 172 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | Q485S- | HEKFIIDTLAASLRSVEKGELRDNGTPKETNSYHRNLKKVLQNHQALIFG |
| | G548A- | RRETPNQENRDNKQLAIYNLEVVKYLEHYFPIKSNVRKNSAADIVYYLK |
| | A737S- | SETIKSTVTHQLENAMRANLLRQGKYQHHDLKADATSENLTNLKRDEA |
| | I1064K | FVMNLITTSAFAANNIRNIIDPEQIKDILGKDDFIRSLKKDAFNKHIYGLFF |
| | | PVGAEMIEPVSLWAMRGAVQQIRNNVAHYKKDALATIFNITKFEYPGAE |
| | | NILFSNTIYKQFFEKELEQLPEAFALQLKSGGVLAQYGFDSLKFLLDVVK |
| | | FHLCRTVVPFAPGFKKVMKGGTGYQNSKQDESFYNLELTAYQLKDDFS |
| | | EEAWSARYFLLKIIYNYLFLPEFINNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDDETPVVHSGIELSVKYGTASLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHDAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 173 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | G548A- | HEKFIIDTLAASLRSVEKGELRDNGTPKETNSYHRNLKKVLQNHQALIFG |
| | A737S- | RRETPNQENRDNKQLAIYNLEVVKYLEHYFPIKSNVRKNSAADIVYYLK |
| | K769E- | SETIKSTVTHQLENAMRANLLRQGKYQHHDLKADATSENLTNLKRDEA |
| | I1064K | FVMNLITTSAFAANNIRNIIDPEQIKDILGKDDFIRSLKKDAFNKHIYGLFF |
| | | PVGAEMIEPVSLWAMRGAVQQIRNNVAHYKKDALATIFNITKFEYPGAE |
| | | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFINNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYENKEQCLQRLQPFIAENSDYTN |
| | | WGDLYVQTDDETPVVHSGIELSVKYGTASLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHDAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 174 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | G548A- | HEKFIIDTLAASLRSVEKGELRDNGTPKETNSYHRNLKKVLQNHQALIFG |
| | A737S- | RRETPNQENRDNKQLAIYNLEVVKYLEHYFPIKSNVRKNSAADIVYYLK |
| | N843S- | SETIKSTVTHQLENAMRANLLRQGKYQHHDLKADATSENLTNLKRDEA |
| | I1064K | FVMNLITTSAFAANNIRNIIDPEQIKDILGKDDFIRSLKKDAFNKHIYGLFF |
| | | PVGAEMIEPVSLWAMRGAVQQIRNNVAHYKKDALATIFNITKFEYPGAE |
| | | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFINNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDDETPVVHSGIELSVKYGTASLLEKLIASDAKFKITENEFDL |
| | | WHSARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHDAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 175 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | G548A- | HEKFIIDTLAASLRSVEKGELRDNGTPKETNSYHRNLKKVLQNHQALIFG |
| | A737S- | RRETPNQENRDNKQLAIYNLEVVKYLEHYFPIKSNVRKNSAADIVYYLK |
| | S919K- | SETIKSTVTHQLENAMRANLLRQGKYQHHDLKADATSENLTNLKRDEA |
| | I1064K | FVMNLITTSAFAANNIRNIIDPEQIKDILGKDDFIRSLKKDAFNKHIYGLFF |
| | | PVGAEMIEPVSLWAMRGAVQQIRNNVAHYKKDALATIFNITKFEYPGAE |
| | | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFINNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDDETPVVHSGIELSVKYGTASLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKKLQNITIEMLGRMAGFVVL |
| | | WERDFQYLDQQRVKQQGGALMDFSHGVPKYEELKPHDAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 176 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | G548A- | HEKFIIDTLAASLRSVEKGELRDNGTPKETNSYHRNLKKVLQNHQALIFG |
| | D574Q- | RRETPNQENRDNKQLAIYNLEVVKYLEHYFPIKSNVRKNSAADIVYYLK |
| | A737S- | SETIKSTVTHQLENAMRANLLRQGKYQHHDLKADATSENLTNLKRDEA |
| | I1064K | FVMNLITTSAFAANNIRNIIDPEQIKDILGKDDFIRSLKKDAFNKHIYGLFF |
| | | PVGAEMIEPVSLWAMRGAVQQIRNNVAHYKKDALATIFNITKFEYPGAE |
| | | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFINNDKVFAQTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDDETPVVHSGIELSVKYGTASLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHDAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 177 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | G548A- | HEKFIIDTLAASLRSVEKGELRDNGTPKETNSYHRNLKKVLQNHQALIFG |
| | A737S- | RRETPNQENRDNKQLAIYNLEVVKYLEHYFPIKSNVRKNSAADIVYYLK |
| | E857M- | SETIKSTVTHQLENAMRANLLRQGKYQHHDLKADATSENLTNLKRDEA |
| | I1064K | FVMNLITTSAFAANNIRNIIDPEQIKDILGKDDFIRSLKKDAFNKHIYGLFF |
| | | PVGAEMIEPVSLWAMRGAVQQIRNNVAHYKKDALATIFNITKFEYPGAE |
| | | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFINNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDDETPVVHSGIELSVKYGTASLLEKLIASDAKFKITENEFDL |
| | | WHNARATIEAMIKKREMYHNLWLEAKEKDDNEKKTRIREKNNYARRF |
| | | VETKGEDYIDICDYIDRYNWLNNKLHFEHLKSLQNITIEMLGRMAGFVV |
| | | LWERDFQYLDQQRVKQQGGALMDFSHGVPKYEELKPHDAYFKRLFLC |
| | | NNYSDTRNHIAHFNYLTKSAANYSLIDLINQLRELLHYDRKLKNAVAKT |
| | | FIDLFDKHGMVLKLKFDSQMHQLQVGSITSKKIFHLGTKKESDNIYTFQV |
| | | PTAYCNMCKQLLEMKN |
| 178 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | R247T- | FLNHKFHLPVKYQKEGQWVQFELSTCILQAVEHQNLDELQPYYDWSKW |
| | K318Q- | YINDKSGKLKKSITNNHIQIGDGESKRQKALLMWETEFTDKGELDLETY |
| | V372L- | HEKFIIDTLAASLRSVEKGELRDNGTPKETNSYHRNLKKVLQNHQALIFG |
| | G548A- | TRETPNQENRDNKQLAIYNLEVVKYLEHYFPIKSNVRKNSAADIVYYLK |
| | A737S- | SETIKSTVTHQLENAMRANLLRQGKYQHHDLKADATSENLTNLKRDEA |
| | S819K- | FVMNLITTSAFAANNIRNIIDPEQIKDILGKDDFIRSLKKDAFNKHIYGLFF |
| | D974Y- | PVGAEMIEPVSLWAMRGAVQQIRNNVAHYKKDALATIFNITKFEYPGAE |
| | I1064K | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDES |
| | | EEAWSARYFLLKIIYNYLFLPEFINNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDDETPVVHSGIELSVKYGTAKLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHYAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 179 | T9G- | MRVSKVKVGVNNKDTMVLMHRSAEKGALIYDTIDKKDRTEEIIPQKKQ |
| | H139Q- | KSFELSIQNKTLLRYEAIKKQKLDRSKYKVVEDIFKYRKLPAKITLEDILP |
| | D184E- | FLNHKFHLPVKYQKEGQWVQFELSTCILQAVEHQNLDELQPYYDWSKW |
| | K318Q- | YINDKSGKLKKSITNNHIQIGDGESKRQKALLMWETEFTDKGELDLETY |
| | V372L- | HEKFIIDTLAASLRSVEKGELRDNGTPKETNSYHRNLKKVLQNHQALIFG |
| | G548A- | RRETPNQENRDNKQLAIYNLEVVKYLEHYFPIKSNVRKNSAADIVYYLK |
| | A737S- | SETIKSTVTHQLENAMRANLLRQGKYQHHDLKADATSENLTNLKRDEA |
| | S819K- | FVMNLITTSAFAANNIRNIIDPEQIKDILGKDDFIRSLKKDAFNKHIYGLFF |
| | D974Y- | PVGAEMIEPVSLWAMRGAVQQIRNNVAHYKKDALATIFNITKFEYPGAE |
| | I1064K | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFTNNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDDETPVVHSGIELSVKYGTAKLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHYAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 180 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | G548A- | HEKFIIDTLAASLRSVEKGELRDNGTPKETNSYHRNLKKVLQNHQALIFG |
| | A737S- | RRETPNQENRDNKQLAIYNLEVVKYLEHYFPIKSNVRKNSAADIVYYLK |
| | S819K- | SETIKSTVTHQLENAMRANLLRQGKYQHHDLKADATSENLTNLKRDEA |
| | D974Y- | FVMNLITTSAFAANNIRNIIDPEQIKDILGKDDFIRSLKKDAFNKHIYGLFF |
| | Q1056L- | PVGAEMIEPVSLWAMRGAVQQIRNNVAHYKKDALATIFNITKFEYPGAE |
| | I1064K | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFINNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDDETPVVHSGIELSVKYGTAKLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHYAYFKRLFLCN |
| | | NYSDTRNHIAHFNYLTKSAANYSLIDLINQLRELLHYDRKLKNAVAKTFI |
| | | DLFDKHGMVLKLKFDSQMHQLLVGSITSKKIFHLGTKKESDNIYTFQVPT |
| | | AYCNMCKQLLEMKN |
| 181 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | G548A- | HEKFIIDTLAASLRSVEKGELRDNGTPKETNSYHRNLKKVLQNHQALIFG |
| | A737S- | RRETPNQENRDNKQLAIYNLEVVKYLEHYFPIKSNVRKNSAADIVYYLK |
| | S819K- | SETIKSTVTHQLENAMRANLLRQGKYQHHDLKADATSENLTNLKRDEA |
| | D974Y- | FVMNLITTSAFAANNIRNIIDPEQIKDILGKDDFIRSLKKDAFNKHIYGLFF |
| | Q1054T- | PVGAEMIEPVSLWAMRGAVQQIRNNVAHYKKDALATIFNITKFEYPGAE |
| | I1064K | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFTNNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDDETPVVHSGIELSVKYGTAKLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHYAYFKRLFLCN |
| | | NYSDTRNHIAHFNYLTKSAANYSLIDLINQLRELLHYDRKLKNAVAKTFI |
| | | DLFDKHGMVLKLKFDSQMHTLQVGSITSKKIFHLGTKKESDNIYTFQVPT |
| | | AYCNMCKQLLEMKN |
| 182 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | A313S- | FLNHKFHLPVKYQKEGQWVQFELSTCILQAVEHQNLDELQPYYDWSKW |
| | K318Q- | YINDKSGKLKKSITNNHIQIGDGESKRQKALLMWETEFTDKGELDLETY |
| | V372L- | HEKFIIDTLAASLRSVEKGELRDNGTPKETNSYHRNLKKVLQNHQALIFG |
| | G548A- | RRETPNQENRDNKQLAIYNLEVVKYLEHYFPIKSNVRKNSAADIVYYLK |
| | A737S- | SETIKSTVTHQLENAMRSNLLRQGKYQHHDLKADATSENLTNLKRDEAF |
| | S819K- | VMNLITTSAFAANNIRNIIDPEQIKDILGKDDFIRSLKKDAFNKHIYGLFFP |
| | D974Y- | VGAEMIEPVSLWAMRGAVQQIRNNVAHYKKDALATIFNITKFEYPGAEN |
| | I1064K | ILFSNTIYKQFFEKELEQLPEAFALQLKSGGVLAQYGFDQLKFLLDVVKF |
| | | HLCRTVVPFAPGFKKVMKGGTGYQNSKQDESFYNLELTAYQLKDDFSE |
| | | EAWSARYFLLKIIYNYLFLPEFINNDKVFADTVAWLLGKNRQQAQSSKN |
| | | KNAYAFADIRLMESSESITDYMAYVQSQWIMEENKKEDDKKEDTRINFE |
| | | KFVLQLFVKGFDTFMKQSAYQFINTPQPQINPTDTNQQQADKLKALEKKI |
| | | CPFCHIQNNKIDPAKDEQIAFYVYCKLLDANHLSTLRNELIKYRASSESEL |
| | | FIHLTEIIELCLLHADNVPADYWQIYKNKEQCLQRLQPFIAENSDYTNWG |
| | | DLYVQTDDETPVVHSGIELSVKYGTAKLLEKLIASDAKFKITENEFDLWH |
| | | NARATIEAMIKKREEYHNLWLEAKEKDDNEKKTRIREKNNYARRFVET |
| | | KGEDYIDICDYIDRYNWLNNKLHFEHLKSLQNITIEMLGRMAGFVVLWE |
| | | RDFQYLDQQRVKQQGGALMDFSHGVPKYEELKPHYAYFKRLFLCNNYS |
| | | DTRNHIAHFNYLTKSAANYSLIDLINQLRELLHYDRKLKNAVAKTFIDLF |
| | | DKHGMVLKLKFDSQMHQLQVGSITSKKIFHLGTKKESDNIYTFQVPTAY |
| | | CNMCKQLLEMKN |
| 183 | H139Q- | MRVSKVKVTVNNKDTMVLMHRSAEKGALIYDTIDKKDRTEEIIPQKKQ |
| | N150Y- | KSFELSIQNKTLLRYEAIKKQKLDRSKYKVVEDIFKYRKLPAKITLEDILP |
| | D184E- | FLNHKFHLPVKYQKEGQWVQFELSTCILQAVEHQNLDELQPYYDWSKW |
| | K318Q- | YIYDKSGKLKKSITNNHIQIGDGESKRQKALLMWETEFTDKGELDLETY |
| | V372L- | HEKFIIDTLAASLRSVEKGELRDNGTPKETNSYHRNLKKVLQNHQALIFG |
| | G548A- | RRETPNQENRDNKQLAIYNLEVVKYLEHYFPIKSNVRKNSAADIVYYLK |
| | A737S- | SETIKSTVTHQLENAMRANLLRQGKYQHHDLKADATSENLTNLKRDEA |
| | S819K- | FVMNLITTSAFAANNIRNIIDPEQIKDILGKDDFIRSLKKDAFNKHIYGLFF |
| | D974Y- | PVGAEMIEPVSLWAMRGAVQQIRNNVAHYKKDALATIFNITKFEYPGAE |
| | I1064K | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFINNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDDETPVVHSGIELSVKYGTAKLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHYAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 184 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | G548A- | HEKFIIDTLAASLRSVEKGELRDNGTPKETNSYHRNLKKVLQNHQALIFG |
| | A737S- | RRETPNQENRDNKQLAIYNLEVVKYLEHYFPIKSNVRKNSAADIVYYLK |
| | S819K- | SETIKSTVTHQLENAMRANLLRQGKYQHHDLKADATSENLTNLKRDEA |
| | D974Y- | FVMNLITTSAFAANNIRNIIDPEQIKDILGKDDFIRSLKKDAFNKHIYGLFF |
| | H1020R- | PVGAEMIEPVSLWAMRGAVQQIRNNVAHYKKDALATIFNITKFEYPGAE |
| | I1064K | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFTNNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDDETPVVHSGIELSVKYGTAKLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHYAYFKRLFLCN |
| | | NYSDTRNHIAHFNYLTKSAANYSLIDLINQLRELLRYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 185 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | G548A- | HEKFIIDTLAASLRSVEKGELRDNGTPKETNSYHRNLKKVLQNHQALIFG |
| | I622N- | RRETPNQENRDNKQLAIYNLEVVKYLEHYFPIKSNVRKNSAADIVYYLK |
| | A737S- | SETIKSTVTHQLENAMRANLLRQGKYQHHDLKADATSENLTNLKRDEA |
| | S819K- | FVMNLITTSAFAANNIRNIIDPEQIKDILGKDDFIRSLKKDAFNKHIYGLFF |
| | D974Y- | PVGAEMIEPVSLWAMRGAVQQIRNNVAHYKKDALATIFNITKFEYPGAE |
| | I1064K | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFINNDKVFADTVAWLLGKNRQQAQSSK |
| | | NKNAYAFADIRLMESSESITDYMAYVQSQWNMEENKKEDDKKEDTRIN |
| | | FEKFVLQLFVKGFDTFMKQSAYQFINTPQPQINPTDTNQQQADKLKALE |
| | | KKICPFCHIQNNKIDPAKDEQIAFYVYCKLLDANHLSTLRNELIKYRASSE |
| | | SELFIHLTEIIELCLLHADNVPADYWQIYKNKEQCLQRLQPFIAENSDYTN |
| | | WGDLYVQTDDETPVVHSGIELSVKYGTAKLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHYAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 186 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | G548A- | HEKFIIDTLAASLRSVEKGELRDNGTPKETNSYHRNLKKVLQNHQALIFG |
| | M623L- | RRETPNQENRDNKQLAIYNLEVVKYLEHYFPIKSNVRKNSAADIVYYLK |
| | A737S- | SETIKSTVTHQLENAMRANLLRQGKYQHHDLKADATSENLTNLKRDEA |
| | S819K- | FVMNLITTSAFAANNIRNIIDPEQIKDILGKDDFIRSLKKDAFNKHIYGLFF |
| | D974Y- | PVGAEMIEPVSLWAMRGAVQQIRNNVAHYKKDALATIFNITKFEYPGAE |
| | I1064K | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFINNDKVFADTVAWLLGKNRQQAQSSK |
| | | NKNAYAFADIRLMESSESITDYMAYVQSQWILEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDDETPVVHSGIELSVKYGTAKLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHYAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 187 | H139Q- | MRVSKVKVTVNNKDTMVLMHRSAEKGALIYDTIDKKDRTEEIIPQKKQ |
| | Q166K- | KSFELSIQNKTLLRYEAIKKQKLDRSKYKVVEDIFKYRKLPAKITLEDILP |
| | D184E- | FLNHKFHLPVKYQKEGQWVQFELSTCILQAVEHQNLDELQPYYDWSKW |
| | K318Q- | YINDKSGKLKKSITNNHIKIGDGESKRQKALLMWETEFTDKGELDLETY |
| | V372L- | HEKFIIDTLAASLRSVEKGELRDNGTPKETNSYHRNLKKVLQNHQALIFG |
| | G548A- | RRETPNQENRDNKQLAIYNLEVVKYLEHYFPIKSNVRKNSAADIVYYLK |
| | A737S- | SETIKSTVTHQLENAMRANLLRQGKYQHHDLKADATSENLTNLKRDEA |
| | S819K- | FVMNLITTSAFAANNIRNIIDPEQIKDILGKDDFIRSLKKDAFNKHIYGLFF |
| | D974Y- | PVGAEMIEPVSLWAMRGAVQQIRNNVAHYKKDALATIFNITKFEYPGAE |
| | I1064K | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFINNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDDETPVVHSGIELSVKYGTAKLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHYAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 188 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | G548A- | HEKFIIDTLAASLRSVEKGELRDNGTPKETNSYHRNLKKVLQNHQALIFG |
| | T726Q- | RRETPNQENRDNKQLAIYNLEVVKYLEHYFPIKSNVRKNSAADIVYYLK |
| | A737S- | SETIKSTVTHQLENAMRANLLRQGKYQHHDLKADATSENLTNLKRDEA |
| | S819K- | FVMNLITTSAFAANNIRNIIDPEQIKDILGKDDFIRSLKKDAFNKHIYGLFF |
| | D974Y- | PVGAEMIEPVSLWAMRGAVQQIRNNVAHYKKDALATIFNITKFEYPGAE |
| | I1064K | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFINNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSQLRNELIKYRASSES |
| | | ELFIHLTEIIELCLLHADNVPADYWQIYKNKEQCLQRLQPFIAENSDYTN |
| | | WGDLYVQTDDETPVVHSGIELSVKYGTAKLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHYAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 189 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | E333H- | YINDKSGKLKKSITNNHIQIGDGESKRQKALLMWETEFTDKGELDLETY |
| | V372L- | HEKFIIDTLAASLRSVEKGELRDNGTPKETNSYHRNLKKVLQNHQALIFG |
| | G548A- | RRETPNQENRDNKQLAIYNLEVVKYLEHYFPIKSNVRKNSAADIVYYLK |
| | A737S- | SETIKSTVTHQLENAMRANLLRQGKYQHHDLKADATSHNLTNLKRDEA |
| | S819K- | FVMNLITTSAFAANNIRNIIDPEQIKDILGKDDFIRSLKKDAFNKHIYGLFF |
| | D974Y- | PVGAEMIEPVSLWAMRGAVQQIRNNVAHYKKDALATIFNITKFEYPGAE |
| | I1064K | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFINNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDDETPVVHSGIELSVKYGTAKLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHYAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 190 | H139Q- | MRVSKVKVTVNNKDTMVLMHRSAEKGALIYDTIDKKDRTEEIIPQKKQ |
| | G154K- | KSFELSIQNKTLLRYEAIKKQKLDRSKYKVVEDIFKYRKLPAKITLEDILP |
| | D184E- | FLNHKFHLPVKYQKEGQWVQFELSTCILQAVEHQNLDELQPYYDWSKW |
| | K318Q- | YINDKSKKLKKSITNNHIQIGDGESKRQKALLMWETEFTDKGELDLETY |
| | V372L- | HEKFIIDTLAASLRSVEKGELRDNGTPKETNSYHRNLKKVLQNHQALIFG |
| | G548A- | RRETPNQENRDNKQLAIYNLEVVKYLEHYFPIKSNVRKNSAADIVYYLK |
| | A737S- | SETIKSTVTHQLENAMRANLLRQGKYQHHDLKADATSENLTNLKRDEA |
| | S819K- | FVMNLITTSAFAANNIRNIIDPEQIKDILGKDDFIRSLKKDAFNKHIYGLFF |
| | D974Y- | PVGAEMIEPVSLWAMRGAVQQIRNNVAHYKKDALATIFNITKFEYPGAE |
| | I1064K | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFINNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDDETPVVHSGIELSVKYGTAKLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHYAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 191 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | G548A- | HEKFIIDTLAASLRSVEKGELRDNGTPKETNSYHRNLKKVLQNHQALIFG |
| | A737S- | RRETPNQENRDNKQLAIYNLEVVKYLEHYFPIKSNVRKNSAADIVYYLK |
| | A762G- | SETIKSTVTHQLENAMRANLLRQGKYQHHDLKADATSENLTNLKRDEA |
| | S819K- | FVMNLITTSAFAANNIRNIIDPEQIKDILGKDDFIRSLKKDAFNKHIYGLFF |
| | D974Y- | PVGAEMIEPVSLWAMRGAVQQIRNNVAHYKKDALATIFNITKFEYPGAE |
| | I1064K | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFINNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPGDYWQIYKNKEQCLQRLQPFIAENSDYTN |
| | | WGDLYVQTDDETPVVHSGIELSVKYGTAKLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHYAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 192 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | A242M- | FLNHKFHLPVKYQKEGQWVQFELSTCILQAVEHQNLDELQPYYDWSKW |
| | K318Q- | YINDKSGKLKKSITNNHIQIGDGESKRQKALLMWETEFTDKGELDLETY |
| | V372L- | HEKFIIDTLAASLRSVEKGELRDNGTPKETNSYHRNLKKVLQNHQMLIFG |
| | G548A- | RRETPNQENRDNKQLAIYNLEVVKYLEHYFPIKSNVRKNSAADIVYYLK |
| | A737S- | SETIKSTVTHQLENAMRANLLRQGKYQHHDLKADATSENLTNLKRDEA |
| | S819K- | FVMNLITTSAFAANNIRNIIDPEQIKDILGKDDFIRSLKKDAFNKHIYGLFF |
| | D974Y- | PVGAEMIEPVSLWAMRGAVQQIRNNVAHYKKDALATIFNITKFEYPGAE |
| | I1064K | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFTNNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDDETPVVHSGIELSVKYGTAKLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHYAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 193 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | G548A- | HEKFIIDTLAASLRSVEKGELRDNGTPKETNSYHRNLKKVLQNHQALIFG |
| | A737S- | RRETPNQENRDNKQLAIYNLEVVKYLEHYFPIKSNVRKNSAADIVYYLK |
| | S819K- | SETIKSTVTHQLENAMRANLLRQGKYQHHDLKADATSENLTNLKRDEA |
| | N909E- | FVMNLITTSAFAANNIRNIIDPEQIKDILGKDDFIRSLKKDAFNKHIYGLFF |
| | D974Y- | PVGAEMIEPVSLWAMRGAVQQIRNNVAHYKKDALATIFNITKFEYPGAE |
| | I1064K | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFINNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDDETPVVHSGIELSVKYGTAKLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLENKLHFEHLKSLQNITIEMLGRMAGFVVL |
| | | WERDFQYLDQQRVKQQGGALMDFSHGVPKYEELKPHYAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 194 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | G548A- | HEKFIIDTLAASLRSVEKGELRDNGTPKETNSYHRNLKKVLQNHQALIFG |
| | A737S- | RRETPNQENRDNKQLAIYNLEVVKYLEHYFPIKSNVRKNSAADIVYYLK |
| | S819K- | SETIKSTVTHQLENAMRANLLRQGKYQHHDLKADATSENLTNLKRDEA |
| | D974Y- | FVMNLITTSAFAANNIRNIIDPEQIKDILGKDDFIRSLKKDAFNKHIYGLFF |
| | D1049G- | PVGAEMIEPVSLWAMRGAVQQIRNNVAHYKKDALATIFNITKFEYPGAE |
| | I1064K | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFINNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDDETPVVHSGIELSVKYGTAKLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHYAYFKRLFLCN |
| | | NYSDTRNHIAHFNYLTKSAANYSLIDLINQLRELLHYDRKLKNAVAKTFI |
| | | DLFDKHGMVLKLKFGSQMHQLQVGSITSKKIFHLGTKKESDNIYTFQVP |
| | | TAYCNMCKQLLEMKN |
| 195 | Q56A- | MRVSKVKVTVNNKDTMVLMHRSAEKGALIYDTIDKKDRTEEIIPQKKQ |
| | H139Q- | KSFELSIANKTLLRYEAIKKQKLDRSKYKVVEDIFKYRKLPAKITLEDILP |
| | D184E- | FLNHKFHLPVKYQKEGQWVQFELSTCILQAVEHQNLDELQPYYDWSKW |
| | K318Q- | YINDKSGKLKKSITNNHIQIGDGESKRQKALLMWETEFTDKGELDLETY |
| | V372L- | HEKFIIDTLAASLRSVEKGELRDNGTPKETNSYHRNLKKVLQNHQALIFG |
| | G548A- | RRETPNQENRDNKQLAIYNLEVVKYLEHYFPIKSNVRKNSAADIVYYLK |
| | A737S- | SETIKSTVTHQLENAMRANLLRQGKYQHHDLKADATSENLTNLKRDEA |
| | S819K- | FVMNLITTSAFAANNIRNIIDPEQIKDILGKDDFIRSLKKDAFNKHIYGLFF |
| | D974Y- | PVGAEMIEPVSLWAMRGAVQQIRNNVAHYKKDALATIFNITKFEYPGAE |
| | I1064K | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFINNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDDETPVVHSGIELSVKYGTAKLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHYAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 196 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | N281K- | FLNHKFHLPVKYQKEGQWVQFELSTCILQAVEHQNLDELQPYYDWSKW |
| | K318Q- | YINDKSGKLKKSITNNHIQIGDGESKRQKALLMWETEFTDKGELDLETY |
| | V372L- | HEKFIIDTLAASLRSVEKGELRDNGTPKETNSYHRNLKKVLQNHQALIFG |
| | G548A- | RRETPNQENRDNKQLAIYNLEVVKYLEHYFPIKSKVRKNSAADIVYYLK |
| | A737S- | SETIKSTVTHQLENAMRANLLRQGKYQHHDLKADATSENLTNLKRDEA |
| | S819K- | FVMNLITTSAFAANNIRNIIDPEQIKDILGKDDFIRSLKKDAFNKHIYGLFF |
| | D974Y- | PVGAEMIEPVSLWAMRGAVQQIRNNVAHYKKDALATIFNITKFEYPGAE |
| | I1064K | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFINNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDDETPVVHSGIELSVKYGTAKLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHYAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 197 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | G548A- | HEKFIIDTLAASLRSVEKGELRDNGTPKETNSYHRNLKKVLQNHQALIFG |
| | W621Q- | RRETPNQENRDNKQLAIYNLEVVKYLEHYFPIKSNVRKNSAADIVYYLK |
| | A737S- | SETIKSTVTHQLENAMRANLLRQGKYQHHDLKADATSENLTNLKRDEA |
| | S819K- | FVMNLITTSAFAANNIRNIIDPEQIKDILGKDDFIRSLKKDAFNKHIYGLFF |
| | D974Y- | PVGAEMIEPVSLWAMRGAVQQIRNNVAHYKKDALATIFNITKFEYPGAE |
| | I1064K | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFINNDKVFADTVAWLLGKNRQQAQSSK |
| | | NKNAYAFADIRLMESSESITDYMAYVQSQQIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDDETPVVHSGIELSVKYGTAKLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHYAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 198 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | G548A- | HEKFIIDTLAASLRSVEKGELRDNGTPKETNSYHRNLKKVLQNHQALIFG |
| | A737S- | RRETPNQENRDNKQLAIYNLEVVKYLEHYFPIKSNVRKNSAADIVYYLK |
| | S819K- | SETIKSTVTHQLENAMRANLLRQGKYQHHDLKADATSENLTNLKRDEA |
| | D974Y- | FVMNLITTSAFAANNIRNIIDPEQIKDILGKDDFIRSLKKDAFNKHIYGLFF |
| | T1032V- | PVGAEMIEPVSLWAMRGAVQQIRNNVAHYKKDALATIFNITKFEYPGAE |
| | I1064K | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFINNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDDETPVVHSGIELSVKYGTAKLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHYAYFKRLFLCN |
| | | NYSDTRNHIAHFNYLTKSAANYSLIDLINQLRELLHYDRKLKNAVAKVFI |
| | | DLFDKHGMVLKLKFDSQMHQLQVGSITSKKIFHLGTKKESDNIYTFQVP |
| | | TAYCNMCKQLLEMKN |
| 199 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | G548A- | HEKFIIDTLAASLRSVEKGELRDNGTPKETNSYHRNLKKVLQNHQALIFG |
| | A737S- | RRETPNQENRDNKQLAIYNLEVVKYLEHYFPIKSNVRKNSAADIVYYLK |
| | S819K- | SETIKSTVTHQLENAMRANLLRQGKYQHHDLKADATSENLTNLKRDEA |
| | D974Y- | FVMNLITTSAFAANNIRNIIDPEQIKDILGKDDFIRSLKKDAFNKHIYGLFF |
| | D1035M- | PVGAEMIEPVSLWAMRGAVQQIRNNVAHYKKDALATIFNITKFEYPGAE |
| | I1064K | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFINNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDDETPVVHSGIELSVKYGTAKLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHYAYFKRLFLCN |
| | | NYSDTRNHIAHFNYLTKSAANYSLIDLINQLRELLHYDRKLKNAVAKTFI |
| | | MLFDKHGMVLKLKFDSQMHQLQVGSITSKKIFHLGTKKESDNIYTFQVP |
| | | TAYCNMCKQLLEMKN |
| 200 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | G548A- | HEKFIIDTLAASLRSVEKGELRDNGTPKETNSYHRNLKKVLQNHQALIFG |
| | A737S- | RRETPNQENRDNKQLAIYNLEVVKYLEHYFPIKSNVRKNSAADIVYYLK |
| | S819K- | SETIKSTVTHQLENAMRANLLRQGKYQHHDLKADATSENLTNLKRDEA |
| | D974Y- | FVMNLITTSAFAANNIRNIIDPEQIKDILGKDDFIRSLKKDAFNKHIYGLFF |
| | I1064K- | PVGAEMIEPVSLWAMRGAVQQIRNNVAHYKKDALATIFNITKFEYPGAE |
| | P1083Q | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFINNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDDETPVVHSGIELSVKYGTAKLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHYAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVQ |
| | | TAYCNMCKQLLEMKN |
| 201 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | A262T- | FLNHKFHLPVKYQKEGQWVQFELSTCILQAVEHQNLDELQPYYDWSKW |
| | K318Q- | YINDKSGKLKKSITNNHIQIGDGESKRQKALLMWETEFTDKGELDLETY |
| | V372L- | HEKFIIDTLAASLRSVEKGELRDNGTPKETNSYHRNLKKVLQNHQALIFG |
| | G548A- | RRETPNQENRDNKQLTIYNLEVVKYLEHYFPIKSNVRKNSAADIVYYLKS |
| | A737S- | ETIKSTVTHQLENAMRANLLRQGKYQHHDLKADATSENLTNLKRDEAF |
| | S819K- | VMNLITTSAFAANNIRNIIDPEQIKDILGKDDFIRSLKKDAFNKHIYGLFFP |
| | D974Y- | VGAEMIEPVSLWAMRGAVQQIRNNVAHYKKDALATIFNITKFEYPGAEN |
| | I1064K | ILFSNTIYKQFFEKELEQLPEAFALQLKSGGVLAQYGFDQLKFLLDVVKF |
| | | HLCRTVVPFAPGFKKVMKGGTGYQNSKQDESFYNLELTAYQLKDDFSE |
| | | EAWSARYFLLKIIYNYLFLPEFINNDKVFADTVAWLLGKNRQQAQSSKN |
| | | KNAYAFADIRLMESSESITDYMAYVQSQWIMEENKKEDDKKEDTRINFE |
| | | KFVLQLFVKGFDTFMKQSAYQFINTPQPQINPTDTNQQQADKLKALEKKI |
| | | CPFCHIQNNKIDPAKDEQIAFYVYCKLLDANHLSTLRNELIKYRASSESEL |
| | | FIHLTEIIELCLLHADNVPADYWQIYKNKEQCLQRLQPFIAENSDYTNWG |
| | | DLYVQTDDETPVVHSGIELSVKYGTAKLLEKLIASDAKFKITENEFDLWH |
| | | NARATIEAMIKKREEYHNLWLEAKEKDDNEKKTRIREKNNYARRFVET |
| | | KGEDYIDICDYIDRYNWLNNKLHFEHLKSLQNITIEMLGRMAGFVVLWE |
| | | RDFQYLDQQRVKQQGGALMDFSHGVPKYEELKPHYAYFKRLFLCNNYS |
| | | DTRNHIAHFNYLTKSAANYSLIDLINQLRELLHYDRKLKNAVAKTFIDLF |
| | | DKHGMVLKLKFDSQMHQLQVGSITSKKIFHLGTKKESDNIYTFQVPTAY |
| | | CNMCKQLLEMKN |
| 202 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | G548A- | HEKFIIDTLAASLRSVEKGELRDNGTPKETNSYHRNLKKVLQNHQALIFG |
| | D631C- | RRETPNQENRDNKQLAIYNLEVVKYLEHYFPIKSNVRKNSAADIVYYLK |
| | A737S- | SETIKSTVTHQLENAMRANLLRQGKYQHHDLKADATSENLTNLKRDEA |
| | S819K- | FVMNLITTSAFAANNIRNIIDPEQIKDILGKDDFIRSLKKDAFNKHIYGLFF |
| | D974Y- | PVGAEMIEPVSLWAMRGAVQQIRNNVAHYKKDALATIFNITKFEYPGAE |
| | I1064K | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFINNDKVFADTVAWLLGKNRQQAQSSK |
| | | NKNAYAFADIRLMESSESITDYMAYVQSQWIMEENKKEDCKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDDETPVVHSGIELSVKYGTAKLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHYAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 203 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | D289T- | FLNHKFHLPVKYQKEGQWVQFELSTCILQAVEHQNLDELQPYYDWSKW |
| | K318Q- | YINDKSGKLKKSITNNHIQIGDGESKRQKALLMWETEFTDKGELDLETY |
| | V372L- | HEKFIIDTLAASLRSVEKGELRDNGTPKETNSYHRNLKKVLQNHQALIFG |
| | G548A- | RRETPNQENRDNKQLAIYNLEVVKYLEHYFPIKSNVRKNSAATIVYYLKS |
| | A737S- | ETIKSTVTHQLENAMRANLLRQGKYQHHDLKADATSENLTNLKRDEAF |
| | S819K- | VMNLITTSAFAANNIRNIIDPEQIKDILGKDDFIRSLKKDAFNKHIYGLFFP |
| | D974Y- | VGAEMIEPVSLWAMRGAVQQIRNNVAHYKKDALATIFNITKFEYPGAEN |
| | I1064K | ILFSNTIYKQFFEKELEQLPEAFALQLKSGGVLAQYGFDQLKFLLDVVKF |
| | | HLCRTVVPFAPGFKKVMKGGTGYQNSKQDESFYNLELTAYQLKDDFSE |
| | | EAWSARYFLLKIIYNYLFLPEFINNDKVFADTVAWLLGKNRQQAQSSKN |
| | | KNAYAFADIRLMESSESITDYMAYVQSQWIMEENKKEDDKKEDTRINFE |
| | | KFVLQLFVKGFDTFMKQSAYQFINTPQPQINPTDTNQQQADKLKALEKKI |
| | | CPFCHIQNNKIDPAKDEQIAFYVYCKLLDANHLSTLRNELIKYRASSESEL |
| | | FIHLTEIIELCLLHADNVPADYWQIYKNKEQCLQRLQPFIAENSDYTNWG |
| | | DLYVQTDDETPVVHSGIELSVKYGTAKLLEKLIASDAKFKITENEFDLWH |
| | | NARATIEAMIKKREEYHNLWLEAKEKDDNEKKTRIREKNNYARRFVET |
| | | KGEDYIDICDYIDRYNWLNNKLHFEHLKSLQNITIEMLGRMAGFVVLWE |
| | | RDFQYLDQQRVKQQGGALMDFSHGVPKYEELKPHYAYFKRLFLCNNYS |
| | | DTRNHIAHFNYLTKSAANYSLIDLINQLRELLHYDRKLKNAVAKTFIDLF |
| | | DKHGMVLKLKFDSQMHQLQVGSITSKKIFHLGTKKESDNIYTFQVPTAY |
| | | CNMCKQLLEMKN |
| 204 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | E543L- | HEKFIIDTLAASLRSVEKGELRDNGTPKETNSYHRNLKKVLQNHQALIFG |
| | G548A- | RRETPNQENRDNKQLAIYNLEVVKYLEHYFPIKSNVRKNSAADIVYYLK |
| | A737S- | SETIKSTVTHQLENAMRANLLRQGKYQHHDLKADATSENLTNLKRDEA |
| | S819K- | FVMNLITTSAFAANNIRNIIDPEQIKDILGKDDFIRSLKKDAFNKHIYGLFF |
| | D974Y- | PVGAEMIEPVSLWAMRGAVQQIRNNVAHYKKDALATIFNITKFEYPGAE |
| | I1064K | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | LEAWSARYFLLKIIYNYLFLPEFINNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDDETPVVHSGIELSVKYGTAKLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHYAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 205 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | G548A- | HEKFIIDTLAASLRSVEKGELRDNGTPKETNSYHRNLKKVLQNHQALIFG |
| | I622K- | RRETPNQENRDNKQLAIYNLEVVKYLEHYFPIKSNVRKNSAADIVYYLK |
| | A737S- | SETIKSTVTHQLENAMRANLLRQGKYQHHDLKADATSENLTNLKRDEA |
| | S819K- | FVMNLITTSAFAANNIRNIIDPEQIKDILGKDDFIRSLKKDAFNKHIYGLFF |
| | D974Y- | PVGAEMIEPVSLWAMRGAVQQIRNNVAHYKKDALATIFNITKFEYPGAE |
| | I1064K | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFINNDKVFADTVAWLLGKNRQQAQSSK |
| | | NKNAYAFADIRLMESSESITDYMAYVQSQWKMEENKKEDDKKEDTRIN |
| | | FEKFVLQLFVKGFDTFMKQSAYQFINTPQPQINPTDTNQQQADKLKALE |
| | | KKICPFCHIQNNKIDPAKDEQIAFYVYCKLLDANHLSTLRNELIKYRASSE |
| | | SELFIHLTEIIELCLLHADNVPADYWQIYKNKEQCLQRLQPFIAENSDYTN |
| | | WGDLYVQTDDETPVVHSGIELSVKYGTAKLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHYAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 206 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | G548A- | HEKFIIDTLAASLRSVEKGELRDNGTPKETNSYHRNLKKVLQNHQALIFG |
| | L684K- | RRETPNQENRDNKQLAIYNLEVVKYLEHYFPIKSNVRKNSAADIVYYLK |
| | A737S- | SETIKSTVTHQLENAMRANLLRQGKYQHHDLKADATSENLTNLKRDEA |
| | S819K- | FVMNLITTSAFAANNIRNIIDPEQIKDILGKDDFIRSLKKDAFNKHIYGLFF |
| | D974Y- | PVGAEMIEPVSLWAMRGAVQQIRNNVAHYKKDALATIFNITKFEYPGAE |
| | I1064K | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFINNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKKKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDDETPVVHSGIELSVKYGTAKLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHYAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 207 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | N265R- | FLNHKFHLPVKYQKEGQWVQFELSTCILQAVEHQNLDELQPYYDWSKW |
| | K318Q- | YINDKSGKLKKSITNNHIQIGDGESKRQKALLMWETEFTDKGELDLETY |
| | V372L- | HEKFIIDTLAASLRSVEKGELRDNGTPKETNSYHRNLKKVLQNHQALIFG |
| | G548A- | RRETPNQENRDNKQLAIYRLEVVKYLEHYFPIKSNVRKNSAADIVYYLK |
| | A737S- | SETIKSTVTHQLENAMRANLLRQGKYQHHDLKADATSENLTNLKRDEA |
| | S819K- | FVMNLITTSAFAANNIRNIIDPEQIKDILGKDDFIRSLKKDAFNKHIYGLFF |
| | D974Y- | PVGAEMIEPVSLWAMRGAVQQIRNNVAHYKKDALATIFNITKFEYPGAE |
| | I1064K | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFINNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDDETPVVHSGIELSVKYGTAKLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHYAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 208 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | S352C- | YINDKSGKLKKSITNNHIQIGDGESKRQKALLMWETEFTDKGELDLETY |
| | V372L- | HEKFIIDTLAASLRSVEKGELRDNGTPKETNSYHRNLKKVLQNHQALIFG |
| | G548A- | RRETPNQENRDNKQLAIYNLEVVKYLEHYFPIKSNVRKNSAADIVYYLK |
| | A737S- | SETIKSTVTHQLENAMRANLLRQGKYQHHDLKADATSENLTNLKRDEA |
| | S819K- | FVMNLITTCAFAANNIRNIIDPEQIKDILGKDDFIRSLKKDAFNKHIYGLFF |
| | S1062P | PVGAEMIEPVSLWAMRGAVQQIRNNVAHYKKDALATIFNITKFEYPGAE |
| | | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFINNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDDETPVVHSGIELSVKYGTAKLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHDAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITPKIIFHLGTKKESDNIYTFQVPT |
| | | AYCNMCKQLLEMKN |
| 209 | T15K- | MRVSKVKVTVNNKDKMVLMHRSAEKGALIYDTIDKKDRTEEIIPQKKQ |
| | H106Q- | KSFELSIQNKTLLRYEAIKKQKLDRSKYKVVEDIFKYRKLPAKITLEDILP |
| | E121N- | FLNHKFQLPVKYQKEGQWVQFNLSTCILQAVKHQNLDELQPYYDWSK |
| | E131K- | WYINDKSGKLKKSITNNHIQIGDGESKRQKALLMWETEFTDKGELDLET |
| | H139Q- | YHEKFIIDTLAASLRSVEKGELRDNGTPKETNSYHRNLKKVLQNHQALIF |
| | D184E- | GRRETPNQENRDNKQLAIYNLEVVKYLEHYFPIKSNVRKNSAADIVYYL |
| | H305K- | KSETIKSTVTKQLENAMRAKLLRQGKYQHHDLKADATSENLTNLKRDE |
| | N314K- | AFVMNLITTCAFAANNIRNIIDPEQIKDILGKDDFIRSLKKDAFNKHIYGLF |
| | K318Q- | FPVGAEMIEPVSLWAMRGAVQQIRNNVAHYKKDALATIFNITKFEYPGA |
| | S352C- | ENILFSNTIYKQFFEKELEQLPEAFALQLKSGGVLAQYGFDQLKFLLDVV |
| | V372L- | KFHLCRTVVPFAPGFKKVMKGGTGYQNSKQDESFYNLELTAYQLKDDF |
| | G548A- | SEEAWSARYFLLKIIYNYLFLPEFTNNDKVFADTVAWLLGKNRQQAQSS |
| | M656L- | KNKNAYAFADIRLMESSESITDYMAYVQSQWIMEENKKEDDKKEDTRIN |
| | A737S- | FEKFVLQLFVKGFDTFLKQSAYQFINTPQPQINPTDTNQQQADKLKALEK |
| | S819K- | KICPFCHIQNNKIDPAKDEQIAFYVYCKLLDANHLSTLRNELIKYRASSES |
| | S1062P | ELFIHLTEIIELCLLHADNVPADYWQIYKNKEQCLQRLQPFIAENSDYTN |
| | | WGDLYVQTDDETPVVHSGIELSVKYGTAKLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHDAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITPKIIFHLGTKKESDNIYTFQVPT |
| | | AYCNMCKQLLEMKN |
| 210 | E131K- | MRVSKVKVTVNNKDTMVLMHRSAEKGALIYDTIDKKDRTEEIIPQKKQ |
| | H139Q- | KSFELSIQNKTLLRYEAIKKQKLDRSKYKVVEDIFKYRKLPAKITLEDILP |
| | D184E- | FLNHKFHLPVKYQKEGQWVQFELSTCILQAVKHQNLDELQPYYDWSKW |
| | E198K- | YINDKSGKLKKSITNNHIQIGDGESKRQKALLMWETEFTDKGELDLETY |
| | H305K- | HKKFIIDTLAASLRSVEKGELRDNGTPKETNSYHRNLKKVLQNHQALIFG |
| | M311V- | RRETPNQENRDNKQLAIYNLEVVKYLEHYFPIKSNVRKNSAADIVYYLK |
| | K318Q- | SETIKSTVTKQLENAVRANLLRQGKYQHHDLKADATSENLTNLKRDEAF |
| | S352C- | VMNLITTCAFAANNIRNIIDPEQIKDILGKDDFIRSLKKDAFNKHIYGLFFP |
| | V372L- | VGAEMIEPVSLWAMRGAVQQIRNNVAHYKKDALATIFNITKFEYPGAEN |
| | G548A- | ILFSNTIYKQFFEKELEQLPEAFALQLKSGGVLAQYGFDQLKFLLDVVKF |
| | A737S- | HLCRTVVPFAPGFKKVMKGGTGYQNSKQDESFYNLELTAYQLKDDFSE |
| | S819K- | EAWSARYFLLKIIYNYLFLPEFINNDKVFADTVAWLLGKNRQQAQSSKN |
| | Y858L- | KNAYAFADIRLMESSESITDYMAYVQSQWIMEENKKEDDKKEDTRINFE |
| | S1062P | KFVLQLFVKGFDTFMKQSAYQFINTPQPQINPTDTNQQQADKLKALEKKI |
| | | CPFCHIQNNKIDPAKDEQIAFYVYCKLLDANHLSTLRNELIKYRASSESEL |
| | | FIHLTEIIELCLLHADNVPADYWQIYKNKEQCLQRLQPFIAENSDYTNWG |
| | | DLYVQTDDETPVVHSGIELSVKYGTAKLLEKLIASDAKFKITENEFDLWH |
| | | NARATIEAMIKKREELHNLWLEAKEKDDNEKKTRIREKNNYARRFVETK |
| | | GEDYIDICDYIDRYNWLNNKLHFEHLKSLQNITIEMLGRMAGFVVLWER |
| | | DFQYLDQQRVKQQGGALMDFSHGVPKYEELKPHDAYFKRLFLCNNYSD |
| | | TRNHIAHFNYLTKSAANYSLIDLINQLRELLHYDRKLKNAVAKTFIDLFD |
| | | KHGMVLKLKFDSQMHQLQVGSITPKIIFHLGTKKESDNIYTFQVPTAYCN |
| | | MCKQLLEMKN |
| 211 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | S352C- | YINDKSGKLKKSITNNHIQIGDGESKRQKALLMWETEFTDKGELDLETY |
| | V372L- | HEKFIIDTLAASLRSVEKGELRDNGTPKETNSYHRNLKKVLQNHQALIFG |
| | G548A- | RRETPNQENRDNKQLAIYNLEVVKYLEHYFPIKSNVRKNSAADIVYYLK |
| | A737S- | SETIKSTVTHQLENAMRANLLRQGKYQHHDLKADATSENLTNLKRDEA |
| | S819K- | FVMNLITTCAFAANNIRNIIDPEQIKDILGKDDFIRSLKKDAFNKHIYGLFF |
| | M927I- | PVGAEMIEPVSLWAMRGAVQQIRNNVAHYKKDALATIFNITKFEYPGAE |
| | A1030S | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFINNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDDETPVVHSGIELSVKYGTAKLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEILGRMAGFVVLW |
| | | ERDFQYLDQQRVKQQGGALMDFSHGVPKYEELKPHDAYFKRLFLCNNY |
| | | SDTRNHIAHFNYLTKSAANYSLIDLINQLRELLHYDRKLKNAVSKTFIDLF |
| | | DKHGMVLKLKFDSQMHQLQVGSITSKIIFHLGTKKESDNIYTFQVPTAYC |
| | | NMCKQLLEMKN |
| 212 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | H305K- | FLNHKFHLPVKYQKEGQWVQFELSTCILQAVEHQNLDELQPYYDWSKW |
| | M311V- | YINDKSGKLKKSITNNHIQIGDGESKRQKALLMWETEFTDKGELDLETY |
| | K318Q- | HEKFIIDTLAASLRSVEKGELRDNGTPKETNSYHRNLKKVLQNHQALIFG |
| | S352C- | RRETPNQENRDNKQLAIYNLEVVKYLEHYFPIKSNVRKNSAADIVYYLK |
| | V372L- | SETIKSTVTKQLENAVRANLLRQGKYQHHDLKADATSENLTNLKRDEAF |
| | S547N- | VMNLITTCAFAANNIRNIIDPEQIKDILGKDDFIRSLKKDAFNKHIYGLFFP |
| | G548A- | VGAEMIEPVSLWAMRGAVQQIRNNVAHYKKDALATIFNITKFEYPGAEN |
| | L647V- | ILFSNTIYKQFFEKELEQLPEAFALQLKSGGVLAQYGFDQLKFLLDVVKF |
| | A737S- | HLCRTVVPFAPGFKKVMKGGTGYQNSKQDESFYNLELTAYQLKDDFSE |
| | S819K- | EAWNARYFLLKIIYNYLFLPEFINNDKVFADTVAWLLGKNRQQAQSSK |
| | Y858L | NKNAYAFADIRLMESSESITDYMAYVQSQWIMEENKKEDDKKEDTRINF |
| | | EKFVLQVFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDDETPVVHSGIELSVKYGTAKLLEKLIASDAKFKITENEFDL |
| | | WHNARATIEAMIKKREELHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHDAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKIIFHLGTKKESDNIYTFQVPT |
| | | AYCNMCKQLLEMKN |
| 213 | H106Q- | MRVSKVKVTVNNKDTMVLMHRSAEKGALIYDTIDKKDRTEEIIPQKKQ |
| | H139Q- | KSFELSIQNKTLLRYEAIKKQKLDRSKYKVVEDIFKYRKLPAKITLEDILP |
| | D184E- | FLNHKFQLPVKYQKEGQWVQFELSTCILQAVEHQNLDELQPYYDWSKW |
| | F200Y- | YINDKSGKLKKSITNNHIQIGDGESKRQKALLMWETEFTDKGELDLETY |
| | K318Q- | HEKYIIDTLAASLRSVEKGELRDNGTPKETNSYHRNLKKVLQNHQALIFG |
| | S352C- | RRETPNQENRDNKQLAIYNLEVVKYLEHYFPIKSNVRKNSAADIVYYLK |
| | V372L- | SETIKSTVTHQLENAMRANLLRQGKYQHHDLKADATSENLTNLKRDEA |
| | G548A- | FVMNLITTCAFAANNIRNIIDPEQIKDILGKDDFIRSLKKDAFNKHIYGLFF |
| | A737S- | PVGAEMIEPVSLWAMRGAVQQIRNNVAHYKKDALATIFNITKFEYPGAE |
| | I1064K | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFINNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDDETPVVHSGIELSVKYGTASLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHDAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 214 | T15K- | MRVSKVKVTVNNKDKMVLMHRSAEKGALIYDTIDKKDRTEEIIPQKKQ |
| | H106Q- | KSFELSIQNKTLLRYEAIKKQKLDRSKYKVVEDIFKYRKLPAKITLEDILP |
| | E131K- | FLNHKFQLPVKYQKEGQWVQFELSTCILQAVKHQNLDELQPYYDWSKW |
| | H139Q- | YINDKSGKLKKSITNNHIQIGDGESKRQKALLMWETEFTDKGELDLETY |
| | D184E- | HEKFIIDTLAASLRSVEKGELRDNGTPKETNSYHRNLKKVLQNHQALIFG |
| | M311V- | RRETPNQENRDNKQLAIYNLEVVKYLEHYFPIKSNVRKNSAADIVYYLK |
| | K318Q- | SETIKSTVTHQLENAVRANLLRQGKYQHHDLKADATSENLTNLKRDEAF |
| | V372L- | VMNLITTSAFAANNIRNIIDPEQIKDILGKDDFIRSLKKDAFNKHIYGLFFP |
| | G548A- | VGAEMIEPVSLWAMRGAVQQIRNNVAHYKKDALATIFNITKFEYPGAEN |
| | A737S- | ILFSNTIYKQFFEKELEQLPEAFALQLKSGGVLAQYGFDQLKFLLDVVKF |
| | I923L | HLCRTVVPFAPGFKKVMKGGTGYQNSKQDESFYNLELTAYQLKDDFSE |
| | | EAWSARYFLLKIIYNYLFLPEFINNDKVFADTVAWLLGKNRQQAQSSKN |
| | | KNAYAFADIRLMESSESITDYMAYVQSQWIMEENKKEDDKKEDTRINFE |
| | | KFVLQLFVKGFDTFMKQSAYQFINTPQPQINPTDTNQQQADKLKALEKKI |
| | | CPFCHIQNNKIDPAKDEQIAFYVYCKLLDANHLSTLRNELIKYRASSESEL |
| | | FIHLTEIIELCLLHADNVPADYWQIYKNKEQCLQRLQPFIAENSDYTNWG |
| | | DLYVQTDDETPVVHSGIELSVKYGTASLLEKLIASDAKFKITENEFDLWH |
| | | NARATIEAMIKKREEYHNLWLEAKEKDDNEKKTRIREKNNYARRFVET |
| | | KGEDYIDICDYIDRYNWLNNKLHFEHLKSLQNLTIEMLGRMAGFVVLWE |
| | | RDFQYLDQQRVKQQGGALMDFSHGVPKYEELKPHDAYFKRLFLCNNYS |
| | | DTRNHIAHFNYLTKSAANYSLIDLINQLRELLHYDRKLKNAVAKTFIDLF |
| | | DKHGMVLKLKFDSQMHQLQVGSITSKIIFHLGTKKESDNIYTFQVPTAYC |
| | | NMCKQLLEMKN |
| 215 | H106Q- | MRVSKVKVTVNNKDTMVLMHRSAEKGALIYDTIDKKDRTEEIIPQKKQ |
| | H139Q- | KSFELSIQNKTLLRYEAIKKQKLDRSKYKVVEDIFKYRKLPAKITLEDILP |
| | D184E- | FLNHKFQLPVKYQKEGQWVQFELSTCILQAVEHQNLDELQPYYDWSKW |
| | M311V- | YINDKSGKLKKSITNNHIQIGDGESKRQKALLMWETEFTDKGELDLETY |
| | K318Q- | HEKFIIDTLAASLRSVEKGELRDNGTPKETNSYHRNLKKVLQNHQALIFG |
| | S352C- | RRETPNQENRDNKQLAIYNLEVVKYLEHYFPIKSNVRKNSAADIVYYLK |
| | V372L- | SETIKSTVTHQLENAVRANLLRQGKYQHHDLKADATSENLTNLKRDEAF |
| | G548A- | VMNLITTCAFAANNIRNIIDPEQIKDILGKDDFIRSLKKDAFNKHIYGLFFP |
| | L647V- | VGAEMIEPVSLWAMRGAVQQIRNNVAHYKKDALATIFNITKFEYPGAEN |
| | A737S- | ILFSNTIYKQFFEKELEQLPEAFALQLKSGGVLAQYGFDQLKFLLDVVKF |
| | S819K- | HLCRTVVPFAPGFKKVMKGGTGYQNSKQDESFYNLELTAYQLKDDFSE |
| | A1030S | EAWSARYFLLKIIYNYLFLPEFINNDKVFADTVAWLLGKNRQQAQSSKN |
| | | KNAYAFADIRLMESSESITDYMAYVQSQWIMEENKKEDDKKEDTRINFE |
| | | KFVLQVFVKGFDTFMKQSAYQFINTPQPQINPTDTNQQQADKLKALEKK |
| | | ICPFCHIQNNKIDPAKDEQIAFYVYCKLLDANHLSTLRNELIKYRASSESE |
| | | LFIHLTEIIELCLLHADNVPADYWQIYKNKEQCLQRLQPFIAENSDYTNW |
| | | GDLYVQTDDETPVVHSGIELSVKYGTAKLLEKLIASDAKFKITENEFDLW |
| | | HNARATIEAMIKKREEYHNLWLEAKEKDDNEKKTRIREKNNYARRFVE |
| | | TKGEDYIDICDYIDRYNWLNNKLHFEHLKSLQNITIEMLGRMAGFVVLW |
| | | ERDFQYLDQQRVKQQGGALMDFSHGVPKYEELKPHDAYFKRLFLCNNY |
| | | SDTRNHIAHFNYLTKSAANYSLIDLINQLRELLHYDRKLKNAVSKTFIDLF |
| | | DKHGMVLKLKFDSQMHQLQVGSITSKIIFHLGTKKESDNIYTFQVPTAYC |
| | | NMCKQLLEMKN |
| 216 | T15K- | MRVSKVKVTVNNKDKMVLMHRSAEKGALIYDTIDKKDRTEEIIPQKKQ |
| | H106Q- | KSFELSIQNKTLLRYEAIKKQKLDRSKYKVVEDIFKYRKLPAKITLEDILP |
| | H139Q- | FLNHKFQLPVKYQKEGQWVQFELSTCILQAVEHQNLDELQPYYDWSKW |
| | D184E- | YINDKSGKLKKSITNNHIQIGDGESKRQKALLMWETEFTDKGELDLETY |
| | H305K- | HEKFIIDTLAASLRSVEKGELRDNGTPKETNSYHRNLKKVLQNHQALIFG |
| | M311V- | RRETPNQENRDNKQLAIYNLEVVKYLEHYFPIKSNVRKNSAADIVYYLK |
| | K318Q- | SETIKSTVTKQLENAVRANLLRQGKYQHHDLKADATSENLTNLKRDEAF |
| | V372L- | VMNLITTSAFAANNIRNIIDPEQIKDILGKDDFIRSLKKDAFNKHIYGLFFP |
| | G548A- | VGAEMIEPVSLWAMRGAVQQIRNNVAHYKKDALATIFNITKFEYPGAEN |
| | A737S- | ILFSNTIYKQFFEKELEQLPEAFALQLKSGGVLAQYGFDQLKFLLDVVKF |
| | I923L- | HLCRTVVPFAPGFKKVMKGGTGYQNSKQDESFYNLELTAYQLKDDFSE |
| | S1062P | EAWSARYFLLKIIYNYLFLPEFINNDKVFADTVAWLLGKNRQQAQSSKN |
| | | KNAYAFADIRLMESSESITDYMAYVQSQWIMEENKKEDDKKEDTRINFE |
| | | KFVLQLFVKGFDTFMKQSAYQFINTPQPQINPTDTNQQQADKLKALEKKI |
| | | CPFCHIQNNKIDPAKDEQIAFYVYCKLLDANHLSTLRNELIKYRASSESEL |
| | | FIHLTEIIELCLLHADNVPADYWQIYKNKEQCLQRLQPFIAENSDYTNWG |
| | | DLYVQTDDETPVVHSGIELSVKYGTASLLEKLIASDAKFKITENEFDLWH |
| | | NARATIEAMIKKREEYHNLWLEAKEKDDNEKKTRIREKNNYARRFVET |
| | | KGEDYIDICDYIDRYNWLNNKLHFEHLKSLQNLTIEMLGRMAGFVVLWE |
| | | RDFQYLDQQRVKQQGGALMDFSHGVPKYEELKPHDAYFKRLFLCNNYS |
| | | DTRNHIAHFNYLTKSAANYSLIDLINQLRELLHYDRKLKNAVAKTFIDLF |
| | | DKHGMVLKLKFDSQMHQLQVGSITPKIIFHLGTKKESDNIYTFQVPTAYC |
| | | NMCKQLLEMKN |
| 217 | H106Q- | MRVSKVKVTVNNKDTMVLMHRSAEKGALIYDTIDKKDRTEEIIPQKKQ |
| | H139Q- | KSFELSIQNKTLLRYEAIKKQKLDRSKYKVVEDIFKYRKLPAKITLEDILP |
| | D184E- | FLNHKFQLPVKYQKEGQWVQFELSTCILQAVEHQNLDELQPYYDWSKW |
| | M311V- | YINDKSGKLKKSITNNHIQIGDGESKRQKALLMWETEFTDKGELDLETY |
| | K318Q- | HEKFIIDTLAASLRSVEKGELRDNGTPKETNSYHRNLKKVLQNHQALIFG |
| | S352C- | RRETPNQENRDNKQLAIYNLEVVKYLEHYFPIKSNVRKNSAADIVYYLK |
| | V372L- | SETIKSTVTHQLENAVRANLLRQGKYQHHDLKADATSENLTNLKRDEAF |
| | G548A- | VMNLITTCAFAANNIRNIIDPEQIKDILGKDDFIRSLKKDAFNKHIYGLFFP |
| | A737S- | VGAEMIEPVSLWAMRGAVQQIRNNVAHYKKDALATIFNITKFEYPGAEN |
| | S819K- | ILFSNTIYKQFFEKELEQLPEAFALQLKSGGVLAQYGFDQLKFLLDVVKF |
| | I1064K | HLCRTVVPFAPGFKKVMKGGTGYQNSKQDESFYNLELTAYQLKDDFSE |
| | | EAWSARYFLLKIIYNYLFLPEFINNDKVFADTVAWLLGKNRQQAQSSKN |
| | | KNAYAFADIRLMESSESITDYMAYVQSQWIMEENKKEDDKKEDTRINFE |
| | | KFVLQLFVKGFDTFMKQSAYQFINTPQPQINPTDTNQQQADKLKALEKKI |
| | | CPFCHIQNNKIDPAKDEQIAFYVYCKLLDANHLSTLRNELIKYRASSESEL |
| | | FIHLTEIIELCLLHADNVPADYWQIYKNKEQCLQRLQPFIAENSDYTNWG |
| | | DLYVQTDDETPVVHSGIELSVKYGTAKLLEKLIASDAKFKITENEFDLWH |
| | | NARATIEAMIKKREEYHNLWLEAKEKDDNEKKTRIREKNNYARRFVET |
| | | KGEDYIDICDYIDRYNWLNNKLHFEHLKSLQNITIEMLGRMAGFVVLWE |
| | | RDFQYLDQQRVKQQGGALMDFSHGVPKYEELKPHDAYFKRLFLCNNYS |
| | | DTRNHIAHFNYLTKSAANYSLIDLINQLRELLHYDRKLKNAVAKTFIDLF |
| | | DKHGMVLKLKFDSQMHQLQVGSITSKKIFHLGTKKESDNIYTFQVPTAY |
| | | CNMCKQLLEMKN |
| 218 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | F200Y- | FLNHKFHLPVKYQKEGQWVQFELSTCILQAVEHQNLDELQPYYDWSKW |
| | K318Q- | YINDKSGKLKKSITNNHIQIGDGESKRQKALLMWETEFTDKGELDLETY |
| | S352C- | HEKYIIDTLAASLRSVEKGELRDNGTPKETNSYHRNLKKVLQNHQALIFG |
| | V372L- | RRETPNQENRDNKQLAIYNLEVVKYLEHYFPIKSNVRKNSAADIVYYLK |
| | G548A- | SETIKSTVTHQLENAMRANLLRQGKYQHHDLKADATSENLTNLKRDEA |
| | L647V- | FVMNLITTCAFAANNIRNIIDPEQIKDILGKDDFIRSLKKDAFNKHIYGLFF |
| | A737S- | PVGAEMIEPVSLWAMRGAVQQIRNNVAHYKKDALATIFNITKFEYPGAE |
| | Y858L- | NILFSNTIYKQFFEKELEQLPEAFALQLKSGGVLAQYGFDQLKFLLDVVK |
| | A1030S- | FHLCRTVVPFAPGFKKVMKGGTGYQNSKQDESFYNLELTAYQLKDDFS |
| | S1062P | EEAWSARYFLLKIIYNYLFLPEFTNNDKVFADTVAWLLGKNRQQAQSSK |
| | | NKNAYAFADIRLMESSESITDYMAYVQSQWIMEENKKEDDKKEDTRINF |
| | | EKFVLQVFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDDETPVVHSGIELSVKYGTASLLEKLIASDAKFKITENEFDL |
| | | WHNARATIEAMIKKREELHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHDAYFKRLFLCN |
| | | NYSDTRNHIAHFNYLTKSAANYSLIDLINQLRELLHYDRKLKNAVSKTFI |
| | | DLFDKHGMVLKLKFDSQMHQLQVGSITPKIIFHLGTKKESDNIYTFQVPT |
| | | AYCNMCKQLLEMKN |
| 219 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | E198K- | FLNHKFHLPVKYQKEGQWVQFELSTCILQAVEHQNLDELQPYYDWSKW |
| | H305K- | YINDKSGKLKKSITNNHIQIGDGESKRQKALLMWETEFTDKGELDLETY |
| | K318Q- | HKKFIIDTLAASLRSVEKGELRDNGTPKETNSYHRNLKKVLQNHQALIFG |
| | S352C- | RRETPNQENRDNKQLAIYNLEVVKYLEHYFPIKSNVRKNSAADIVYYLK |
| | V372L- | SETIKSTVTKQLENAMRANLLRQGKYQHHDLKADATSENLTNLKRDEA |
| | G548A- | FVMNLITTCAFAANNIRNIIDPEQIKDILGKDDFIRSLKKDAFNKHIYGLFF |
| | A737S- | PVGAEMIEPVSLWAMRGAVQQIRNNVAHYKKDALATIFNITKFEYPGAE |
| | S819K | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFINNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDDETPVVHSGIELSVKYGTAKLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHDAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKIIFHLGTKKESDNIYTFQVPT |
| | | AYCNMCKQLLEMKN |
| 220 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | H305K- | FLNHKFHLPVKYQKEGQWVQFELSTCILQAVEHQNLDELQPYYDWSKW |
| | M311V- | YINDKSGKLKKSITNNHIQIGDGESKRQKALLMWETEFTDKGELDLETY |
| | K318Q- | HEKFIIDTLAASLRSVEKGELRDNGTPKETNSYHRNLKKVLQNHQALIFG |
| | S352C- | RRETPNQENRDNKQLAIYNLEVVKYLEHYFPIKSNVRKNSAADIVYYLK |
| | V372L- | SETIKSTVTKQLENAVRANLLRQGKYQHHDLKADATSENLTNLKRDEAF |
| | G548A- | VMNLITTCAFAANNIRNIIDPEQIKDILGKDDFIRSLKKDAFNKHIYGLFFP |
| | A737S- | VGAEMIEPVSLWAMRGAVQQIRNNVAHYKKDALATIFNITKFEYPGAEN |
| | S819K- | ILFSNTIYKQFFEKELEQLPEAFALQLKSGGVLAQYGFDQLKFLLDVVKF |
| | S1062P | HLCRTVVPFAPGFKKVMKGGTGYQNSKQDESFYNLELTAYQLKDDFSE |
| | | EAWSARYFLLKIIYNYLFLPEFTNNDKVFADTVAWLLGKNRQQAQSSKN |
| | | KNAYAFADIRLMESSESITDYMAYVQSQWIMEENKKEDDKKEDTRINFE |
| | | KFVLQLFVKGFDTFMKQSAYQFINTPQPQINPTDTNQQQADKLKALEKKI |
| | | CPFCHIQNNKIDPAKDEQIAFYVYCKLLDANHLSTLRNELIKYRASSESEL |
| | | FIHLTEIIELCLLHADNVPADYWQIYKNKEQCLQRLQPFIAENSDYTNWG |
| | | DLYVQTDDETPVVHSGIELSVKYGTAKLLEKLIASDAKFKITENEFDLWH |
| | | NARATIEAMIKKREEYHNLWLEAKEKDDNEKKTRIREKNNYARRFVET |
| | | KGEDYIDICDYIDRYNWLNNKLHFEHLKSLQNITIEMLGRMAGFVVLWE |
| | | RDFQYLDQQRVKQQGGALMDFSHGVPKYEELKPHDAYFKRLFLCNNYS |
| | | DTRNHIAHFNYLTKSAANYSLIDLINQLRELLHYDRKLKNAVAKTFIDLF |
| | | DKHGMVLKLKFDSQMHQLQVGSITPKIIFHLGTKKESDNIYTFQVPTAYC |
| | | NMCKQLLEMKN |
| 221 | H106Q- | MRVSKVKVTVNNKDTMVLMHRSAEKGALIYDTIDKKDRTEEIIPQKKQ |
| | E131K- | KSFELSIQNKTLLRYEAIKKQKLDRSKYKVVEDIFKYRKLPAKITLEDILP |
| | H139Q- | FLNHKFQLPVKYQKEGQWVQFELSTCILQAVKHQNLDELQPYYDWSKW |
| | D184E- | YINDKSGKLKKSITNNHIQIGDGESKRQKALLMWETEFTDKGELDLETY |
| | F200Y- | HEKYIIDTLAASLRSVEKGELRDNGTPKETNSYHRNLKKVLQNHQALIFG |
| | M311V- | RRETPNQENRDNKQLAIYNLEVVKYLEHYFPIKSNVRKNSAADIVYYLK |
| | K318Q- | SETIKSTVTHQLENAVRANLLRQGKYQHHDLKADATSENLTNLKRDEAF |
| | V372L- | VMNLITTSAFAANNIRNIIDPEQIKDILGKDDFIRSLKKDAFNKHIYGLFFP |
| | G548A- | VGAEMIEPVSLWAMRGAVQQIRNNVAHYKKDALATIFNITKFEYPGAEN |
| | A737S | ILFSNTIYKQFFEKELEQLPEAFALQLKSGGVLAQYGFDQLKFLLDVVKF |
| | | HLCRTVVPFAPGFKKVMKGGTGYQNSKQDESFYNLELTAYQLKDDFSE |
| | | EAWSARYFLLKIIYNYLFLPEFINNDKVFADTVAWLLGKNRQQAQSSKN |
| | | KNAYAFADIRLMESSESITDYMAYVQSQWIMEENKKEDDKKEDTRINFE |
| | | KFVLQLFVKGFDTFMKQSAYQFINTPQPQINPTDTNQQQADKLKALEKKI |
| | | CPFCHIQNNKIDPAKDEQIAFYVYCKLLDANHLSTLRNELIKYRASSESEL |
| | | FIHLTEIIELCLLHADNVPADYWQIYKNKEQCLQRLQPFIAENSDYTNWG |
| | | DLYVQTDDETPVVHSGIELSVKYGTASLLEKLIASDAKFKITENEFDLWH |
| | | NARATIEAMIKKREEYHNLWLEAKEKDDNEKKTRIREKNNYARRFVET |
| | | KGEDYIDICDYIDRYNWLNNKLHFEHLKSLQNITIEMLGRMAGFVVLWE |
| | | RDFQYLDQQRVKQQGGALMDFSHGVPKYEELKPHDAYFKRLFLCNNYS |
| | | DTRNHIAHFNYLTKSAANYSLIDLINQLRELLHYDRKLKNAVAKTFIDLF |
| | | DKHGMVLKLKFDSQMHQLQVGSITSKIIFHLGTKKESDNIYTFQVPTAYC |
| | | NMCKQLLEMKN |
| 222 | T15K- | MRVSKVKVTVNNKDKMVLMHRSAEKGALIYDTIDKKDRTEEIIPQKKQ |
| | E121N- | KSFELSIQNKTLLRYEAIKKQKLDRSKYKVVEDIFKYRKLPAKITLEDILP |
| | E131K- | FLNHKFHLPVKYQKEGQWVQFNLSTCILQAVKHQNLDELQPYYDWSK |
| | H139Q- | WYINDKSGKLKKSITNNHIQIGDGESKRQKALLMWETEFTDKGELDLET |
| | D184E- | YHEKYIIDTLAASLRSVEKGELRDNGTPKETNSYHRNLKKVLQNHQALIF |
| | F200Y- | GRRETPNQENRDNKQLAIYNLEVVKYLEHYFPIKSNVRKNSAADIVYYL |
| | K318Q- | KSETIKSTVTHQLENAMRANLLRQGKYQHHDLKADATSENLTNLKRDE |
| | V372L- | AFVMNLITTSAFAANNIRNIIDPEQIKDILGKDDFIRSLKKDAFNKHIYGLF |
| | G548A- | FPVGAEMIEPVSLWAMRGAVQQIRNNVAHYKKDALATIFNITKFEYPGA |
| | A737S- | ENILFSNTIYKQFFEKELEQLPEAFALQLKSGGVLAQYGFDQLKFLLDVV |
| | I1064K | KFHLCRTVVPFAPGFKKVMKGGTGYQNSKQDESFYNLELTAYQLKDDF |
| | | SEEAWSARYFLLKIIYNYLFLPEFTNNDKVFADTVAWLLGKNRQQAQSS |
| | | KNKNAYAFADIRLMESSESITDYMAYVQSQWIMEENKKEDDKKEDTRIN |
| | | FEKFVLQLFVKGFDTFMKQSAYQFINTPQPQINPTDTNQQQADKLKALE |
| | | KKICPFCHIQNNKIDPAKDEQIAFYVYCKLLDANHLSTLRNELIKYRASSE |
| | | SELFIHLTEIIELCLLHADNVPADYWQIYKNKEQCLQRLQPFIAENSDYTN |
| | | WGDLYVQTDDETPVVHSGIELSVKYGTASLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHDAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 223 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | S352C- | YINDKSGKLKKSITNNHIQIGDGESKRQKALLMWETEFTDKGELDLETY |
| | V372L- | HEKFIIDTLAASLRSVEKGELRDNGTPKETNSYHRNLKKVLQNHQALIFG |
| | G548A- | RRETPNQENRDNKQLAIYNLEVVKYLEHYFPIKSNVRKNSAADIVYYLK |
| | A737S- | SETIKSTVTHQLENAMRANLLRQGKYQHHDLKADATSENLTNLKRDEA |
| | S819K | FVMNLITTCAFAANNIRNIIDPEQIKDILGKDDFIRSLKKDAFNKHIYGLFF |
| | | PVGAEMIEPVSLWAMRGAVQQIRNNVAHYKKDALATIFNITKFEYPGAE |
| | | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFINNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDDETPVVHSGIELSVKYGTAKLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHDAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKIIFHLGTKKESDNIYTFQVPT |
| | | AYCNMCKQLLEMKN |
| 224 | H106Q- | MRVSKVKVTVNNKDTMVLMHRSAEKGALIYDTIDKKDRTEEIIPQKKQ |
| | H139Q- | KSFELSIQNKTLLRYEAIKKQKLDRSKYKVVEDIFKYRKLPAKITLEDILP |
| | D184E- | FLNHKFQLPVKYQKEGQWVQFELSTCILQAVEHQNLDELQPYYDWSKW |
| | M311V- | YINDKSGKLKKSITNNHIQIGDGESKRQKALLMWETEFTDKGELDLETY |
| | K318Q- | HEKFIIDTLAASLRSVEKGELRDNGTPKETNSYHRNLKKVLQNHQALIFG |
| | V372L- | RRETPNQENRDNKQLAIYNLEVVKYLEHYFPIKSNVRKNSAADIVYYLK |
| | G548A- | SETIKSTVTHQLENAVRANLLRQGKYQHHDLKADATSENLTNLKRDEAF |
| | D569H- | VMNLITTSAFAANNIRNIIDPEQIKDILGKDDFIRSLKKDAFNKHIYGLFFP |
| | N722R- | VGAEMIEPVSLWAMRGAVQQIRNNVAHYKKDALATIFNITKFEYPGAEN |
| | A737S- | ILFSNTIYKQFFEKELEQLPEAFALQLKSGGVLAQYGFDQLKFLLDVVKF |
| | S807R- | HLCRTVVPFAPGFKKVMKGGTGYQNSKQDESFYNLELTAYQLKDDFSE |
| | S819K- | EAWSARYFLLKIIYNYLFLPEFTNNHKVFADTVAWLLGKNRQQAQSSKN |
| | D974Y- | KNAYAFADIRLMESSESITDYMAYVQSQWIMEENKKEDDKKEDTRINFE |
| | I1064K | KFVLQLFVKGFDTFMKQSAYQFINTPQPQINPTDTNQQQADKLKALEKKI |
| | | CPFCHIQNNKIDPAKDEQIAFYVYCKLLDARHLSTLRNELIKYRASSESEL |
| | | FIHLTEIIELCLLHADNVPADYWQIYKNKEQCLQRLQPFIAENSDYTNWG |
| | | DLYVQTDDETPVVHRGIELSVKYGTAKLLEKLIASDAKFKITENEFDLWH |
| | | NARATIEAMIKKREEYHNLWLEAKEKDDNEKKTRIREKNNYARRFVET |
| | | KGEDYIDICDYIDRYNWLNNKLHFEHLKSLQNITIEMLGRMAGFVVLWE |
| | | RDFQYLDQQRVKQQGGALMDFSHGVPKYEELKPHYAYFKRLFLCNNYS |
| | | DTRNHIAHFNYLTKSAANYSLIDLINQLRELLHYDRKLKNAVAKTFIDLF |
| | | DKHGMVLKLKFDSQMHQLQVGSITSKKIFHLGTKKESDNIYTFQVPTAY |
| | | CNMCKQLLEMKN |
| 225 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | F200Y- | FLNHKFHLPVKYQKEGQWVQFELSTCILQAVEHQNLDELQPYYDWSKW |
| | K318Q- | YINDKSGKLKKSITNNHIQIGDGESKRQKALLMWETEFTDKGELDLETY |
| | V372L- | HEKYIIDTLAASLRSVEKGELRDNGTPKETNSYHRNLKKVLQNHQALIFG |
| | G548A- | RRETPNQENRDNKQLAIYNLEVVKYLEHYFPIKSNVRKNSAADIVYYLK |
| | K717M- | SETIKSTVTHQLENAMRANLLRQGKYQHHDLKADATSENLTNLKRDEA |
| | A737S- | FVMNLITTSAFAANNIRNIIDPEQIKDILGKDDFIRSLKKDAFNKHIYGLFF |
| | D800K- | PVGAEMIEPVSLWAMRGAVQQIRNNVAHYKKDALATIFNITKFEYPGAE |
| | S819K- | NILFSNTIYKQFFEKELEQLPEAFALQLKSGGVLAQYGFDQLKFLLDVVK |
| | N871K- | FHLCRTVVPFAPGFKKVMKGGTGYQNSKQDESFYNLELTAYQLKDDES |
| | D974Y- | EEAWSARYFLLKIIYNYLFLPEFINNDKVFADTVAWLLGKNRQQAQSSK |
| | I1064K | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCMLLDANHLSTLRNELIKYRASSES |
| | | ELFIHLTEIIELCLLHADNVPADYWQIYKNKEQCLQRLQPFIAENSDYTN |
| | | WGDLYVQTDKETPVVHSGIELSVKYGTAKLLEKLIASDAKFKITENEFDL |
| | | WHNARATIEAMIKKREEYHNLWLEAKEKDDKEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHYAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 226 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | V501T- | HEKFIIDTLAASLRSVEKGELRDNGTPKETNSYHRNLKKVLQNHQALIFG |
| | L537R- | RRETPNQENRDNKQLAIYNLEVVKYLEHYFPIKSNVRKNSAADIVYYLK |
| | G548A- | SETIKSTVTHQLENAMRANLLRQGKYQHHDLKADATSENLTNLKRDEA |
| | N567S- | FVMNLITTSAFAANNIRNIIDPEQIKDILGKDDFIRSLKKDAFNKHIYGLFF |
| | A737S- | PVGAEMIEPVSLWAMRGAVQQIRNNVAHYKKDALATIFNITKFEYPGAE |
| | S807R- | NILFSNTIYKQFFEKELEQLPEAFALQLKSGGVLAQYGFDQLKFLLDVVK |
| | S819K- | FHLCRTTVPFAPGFKKVMKGGTGYQNSKQDESFYNLELTAYQRKDDFSE |
| | D974Y- | EAWSARYFLLKIIYNYLFLPEFTSNDKVFADTVAWLLGKNRQQAQSSKN |
| | I1064K | KNAYAFADIRLMESSESITDYMAYVQSQWIMEENKKEDDKKEDTRINFE |
| | | KFVLQLFVKGFDTFMKQSAYQFINTPQPQINPTDTNQQQADKLKALEKKI |
| | | CPFCHIQNNKIDPAKDEQIAFYVYCKLLDANHLSTLRNELIKYRASSESEL |
| | | FIHLTEIIELCLLHADNVPADYWQIYKNKEQCLQRLQPFIAENSDYTNWG |
| | | DLYVQTDDETPVVHRGIELSVKYGTAKLLEKLIASDAKFKITENEFDLWH |
| | | NARATIEAMIKKREEYHNLWLEAKEKDDNEKKTRIREKNNYARRFVET |
| | | KGEDYIDICDYIDRYNWLNNKLHFEHLKSLQNITIEMLGRMAGFVVLWE |
| | | RDFQYLDQQRVKQQGGALMDFSHGVPKYEELKPHYAYFKRLFLCNNYS |
| | | DTRNHIAHFNYLTKSAANYSLIDLINQLRELLHYDRKLKNAVAKTFIDLF |
| | | DKHGMVLKLKFDSQMHQLQVGSITSKKIFHLGTKKESDNIYTFQVPTAY |
| | | CNMCKQLLEMKN |
| 227 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | F200Y- | FLNHKFHLPVKYQKEGQWVQFELSTCILQAVEHQNLDELQPYYDWSKW |
| | K318Q- | YINDKSGKLKKSITNNHIQIGDGESKRQKALLMWETEFTDKGELDLETY |
| | V372L- | HEKYIIDTLAASLRSVEKGELRDNGTPKETNSYHRNLKKVLQNHQALIFG |
| | G548A- | RRETPNQENRDNKQLAIYNLEVVKYLEHYFPIKSNVRKNSAADIVYYLK |
| | D569H- | SETIKSTVTHQLENAMRANLLRQGKYQHHDLKADATSENLTNLKRDEA |
| | A737S- | FVMNLITTSAFAANNIRNIIDPEQIKDILGKDDFIRSLKKDAFNKHIYGLFF |
| | S819K- | PVGAEMIEPVSLWAMRGAVQQIRNNVAHYKKDALATIFNITKFEYPGAE |
| | D974Y- | NILFSNTIYKQFFEKELEQLPEAFALQLKSGGVLAQYGFDQLKFLLDVVK |
| | I1064K | FHLCRTVVPFAPGFKKVMKGGTGYQNSKQDESFYNLELTAYQLKDDFS |
| | | EEAWSARYFLLKIIYNYLFLPEFTNNHKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDDETPVVHSGIELSVKYGTAKLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHYAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 228 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | G548A- | HEKFIIDTLAASLRSVEKGELRDNGTPKETNSYHRNLKKVLQNHQALIFG |
| | N567S- | RRETPNQENRDNKQLAIYNLEVVKYLEHYFPIKSNVRKNSAADIVYYLK |
| | A737S- | SETIKSTVTHQLENAMRANLLRQGKYQHHDLKADATSENLTNLKRDEA |
| | D800K- | FVMNLITTSAFAANNIRNIIDPEQIKDILGKDDFIRSLKKDAFNKHIYGLFF |
| | S819K- | PVGAEMIEPVSLWAMRGAVQQIRNNVAHYKKDALATIFNITKFEYPGAE |
| | D974Y- | NILFSNTIYKQFFEKELEQLPEAFALQLKSGGVLAQYGFDQLKFLLDVVK |
| | I1064K | FHLCRTVVPFAPGFKKVMKGGTGYQNSKQDESFYNLELTAYQLKDDFS |
| | | EEAWSARYFLLKIIYNYLFLPEFTSNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDKETPVVHSGIELSVKYGTAKLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHYAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 229 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | G548A- | HEKFIIDTLAASLRSVEKGELRDNGTPKETNSYHRNLKKVLQNHQALIFG |
| | D569H- | RRETPNQENRDNKQLAIYNLEVVKYLEHYFPIKSNVRKNSAADIVYYLK |
| | A737S- | SETIKSTVTHQLENAMRANLLRQGKYQHHDLKADATSENLTNLKRDEA |
| | S807R- | FVMNLITTSAFAANNIRNIIDPEQIKDILGKDDFIRSLKKDAFNKHIYGLFF |
| | S819K- | PVGAEMIEPVSLWAMRGAVQQIRNNVAHYKKDALATIFNITKFEYPGAE |
| | S919K- | NILFSNTIYKQFFEKELEQLPEAFALQLKSGGVLAQYGFDQLKFLLDVVK |
| | D974Y- | FHLCRTVVPFAPGFKKVMKGGTGYQNSKQDESFYNLELTAYQLKDDFS |
| | I1064K | EEAWSARYFLLKIIYNYLFLPEFTNNHKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDDETPVVHRGIELSVKYGTAKLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKKLQNITIEMLGRMAGFVVL |
| | | WERDFQYLDQQRVKQQGGALMDFSHGVPKYEELKPHYAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 230 | H106Q- | MRVSKVKVTVNNKDTMVLMHRSAEKGALIYDTIDKKDRTEEIIPQKKQ |
| | H139Q- | KSFELSIQNKTLLRYEAIKKQKLDRSKYKVVEDIFKYRKLPAKITLEDILP |
| | D184E- | FLNHKFQLPVKYQKEGQWVQFELSTCILQAVEHQNLDELQPYYDWSKW |
| | F200Y- | YINDKSGKLKKSITNNHIQIGDGESKRQKALLMWETEFTDKGELDLETY |
| | K318Q- | HEKYIIDTLAASLRSVEKGELRDNGTPKETNSYHRNLKKVLQNHQALIFG |
| | S352C- | RRETPNQENRDNKQLAIYNLEVVKYLEHYFPIKSNVRKNSAADIVYYLK |
| | V372L- | SETIKSTVTHQLENAMRANLLRQGKYQHHDLKADATSENLTNLKRDEA |
| | L537R- | FVMNLITTCAFAANNIRNIIDPEQIKDILGKDDFIRSLKKDAFNKHIYGLFF |
| | G548A- | PVGAEMIEPVSLWAMRGAVQQIRNNVAHYKKDALATIFNITKFEYPGAE |
| | N567S- | NILFSNTIYKQFFEKELEQLPEAFALQLKSGGVLAQYGFDQLKFLLDVVK |
| | A737S- | FHLCRTVVPFAPGFKKVMKGGTGYQNSKQDESFYNLELTAYQRKDDFS |
| | D800K- | EEAWSARYFLLKIIYNYLFLPEFTSNDKVFADTVAWLLGKNRQQAQSSK |
| | S819K- | NKNAYAFADIRLMESSESITDYMAYVQSQWIMEENKKEDDKKEDTRINF |
| | D974Y- | EKFVLQLFVKGFDTFMKQSAYQFINTPQPQINPTDTNQQQADKLKALEK |
| | I1064K | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDKETPVVHSGIELSVKYGTAKLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHYAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 231 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | M311V- | FLNHKFHLPVKYQKEGQWVQFELSTCILQAVEHQNLDELQPYYDWSKW |
| | K318Q- | YINDKSGKLKKSITNNHIQIGDGESKRQKALLMWETEFTDKGELDLETY |
| | S352C- | HEKFIIDTLAASLRSVEKGELRDNGTPKETNSYHRNLKKVLQNHQALIFG |
| | V372L- | RRETPNQENRDNKQLAIYNLEVVKYLEHYFPIKSNVRKNSAADIVYYLK |
| | V501T- | SETIKSTVTHQLENAVRANLLRQGKYQHHDLKADATSENLTNLKRDEAF |
| | G548A- | VMNLITTCAFAANNIRNIIDPEQIKDILGKDDFIRSLKKDAFNKHIYGLFFP |
| | Q585L- | VGAEMIEPVSLWAMRGAVQQIRNNVAHYKKDALATIFNITKFEYPGAEN |
| | A705T- | ILFSNTIYKQFFEKELEQLPEAFALQLKSGGVLAQYGFDQLKFLLDVVKF |
| | N722R- | HLCRTTVPFAPGFKKVMKGGTGYQNSKQDESFYNLELTAYQLKDDFSEE |
| | A737S- | AWSARYFLLKIIYNYLFLPEFINNDKVFADTVAWLLGKNRLQAQSSKNK |
| | W765N- | NAYAFADIRLMESSESITDYMAYVQSQWIMEENKKEDDKKEDTRINFEK |
| | S807R- | FVLQLFVKGFDTFMKQSAYQFINTPQPQINPTDTNQQQADKLKALEKKIC |
| | S819K- | PFCHIQNNKIDPTKDEQIAFYVYCKLLDARHLSTLRNELIKYRASSESELFI |
| | D974Y- | HLTEIIELCLLHADNVPADYNQIYKNKEQCLQRLQPFIAENSDYTNWGDL |
| | I1064K | YVQTDDETPVVHRGIELSVKYGTAKLLEKLIASDAKFKITENEFDLWHN |
| | | ARATIEAMIKKREEYHNLWLEAKEKDDNEKKTRIREKNNYARRFVETK |
| | | GEDYIDICDYIDRYNWLNNKLHFEHLKSLQNITIEMLGRMAGFVVLWER |
| | | DFQYLDQQRVKQQGGALMDFSHGVPKYEELKPHYAYFKRLFLCNNYSD |
| | | TRNHIAHFNYLTKSAANYSLIDLINQLRELLHYDRKLKNAVAKTFIDLFD |
| | | KHGMVLKLKFDSQMHQLQVGSITSKKIFHLGTKKESDNIYTFQVPTAYC |
| | | NMCKQLLEMKN |
| 232 | H106Q- | MRVSKVKVTVNNKDTMVLMHRSAEKGALIYDTIDKKDRTEEIIPQKKQ |
| | H139Q- | KSFELSIQNKTLLRYEAIKKQKLDRSKYKVVEDIFKYRKLPAKITLEDILP |
| | D184E- | FLNHKFQLPVKYQKEGQWVQFELSTCILQAVEHQNLDELQPYYDWSKW |
| | F200Y- | YINDKSGKLKKSITNNHIQIGDGESKRQKALLMWETEFTDKGELDLETY |
| | M311V- | HEKYIIDTLAASLRSVEKGELRDNGTPKETNSYHRNLKKVLQNHQALIFG |
| | K318Q- | RRETPNQENRDNKQLAIYNLEVVKYLEHYFPIKSNVRKNSAADIVYYLK |
| | V372L- | SETIKSTVTHQLENAVRANLLRQGKYQHHDLKADATSENLTNLKRDEAF |
| | G548A- | VMNLITTSAFAANNIRNIIDPEQIKDILGKDDFIRSLKKDAFNKHIYGLFFP |
| | N567S- | VGAEMIEPVSLWAMRGAVQQIRNNVAHYKKDALATIFNITKFEYPGAEN |
| | D569H- | ILFSNTIYKQFFEKELEQLPEAFALQLKSGGVLAQYGFDQLKFLLDVVKF |
| | A737S- | HLCRTVVPFAPGFKKVMKGGTGYQNSKQDESFYNLELTAYQLKDDFSE |
| | S819K- | EAWSARYFLLKIIYNYLFLPEFTSNHKVFADTVAWLLGKNRQQAQSSKN |
| | D974Y- | KNAYAFADIRLMESSESITDYMAYVQSQWIMEENKKEDDKKEDTRINFE |
| | I1064K | KFVLQLFVKGFDTFMKQSAYQFINTPQPQINPTDTNQQQADKLKALEKKI |
| | | CPFCHIQNNKIDPAKDEQIAFYVYCKLLDANHLSTLRNELIKYRASSESEL |
| | | FIHLTEIIELCLLHADNVPADYWQIYKNKEQCLQRLQPFIAENSDYTNWG |
| | | DLYVQTDDETPVVHSGIELSVKYGTAKLLEKLIASDAKFKITENEFDLWH |
| | | NARATIEAMIKKREEYHNLWLEAKEKDDNEKKTRIREKNNYARRFVET |
| | | KGEDYIDICDYIDRYNWLNNKLHFEHLKSLQNITIEMLGRMAGFVVLWE |
| | | RDFQYLDQQRVKQQGGALMDFSHGVPKYEELKPHYAYFKRLFLCNNYS |
| | | DTRNHIAHFNYLTKSAANYSLIDLINQLRELLHYDRKLKNAVAKTFIDLF |
| | | DKHGMVLKLKFDSQMHQLQVGSITSKKIFHLGTKKESDNIYTFQVPTAY |
| | | CNMCKQLLEMKN |
| 233 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | V501T- | HEKFIIDTLAASLRSVEKGELRDNGTPKETNSYHRNLKKVLQNHQALIFG |
| | G548A- | RRETPNQENRDNKQLAIYNLEVVKYLEHYFPIKSNVRKNSAADIVYYLK |
| | D569H- | SETIKSTVTHQLENAMRANLLRQGKYQHHDLKADATSENLTNLKRDEA |
| | A737S- | FVMNLITTSAFAANNIRNIIDPEQIKDILGKDDFIRSLKKDAFNKHIYGLFF |
| | W765N- | PVGAEMIEPVSLWAMRGAVQQIRNNVAHYKKDALATIFNITKFEYPGAE |
| | S807R- | NILFSNTIYKQFFEKELEQLPEAFALQLKSGGVLAQYGFDQLKFLLDVVK |
| | S819K- | FHLCRTTVPFAPGFKKVMKGGTGYQNSKQDESFYNLELTAYQLKDDFSE |
| | N871K- | EAWSARYFLLKIIYNYLFLPEFTNNHKVFADTVAWLLGKNRQQAQSSKN |
| | D974Y- | KNAYAFADIRLMESSESITDYMAYVQSQWIMEENKKEDDKKEDTRINFE |
| | I1064K | KFVLQLFVKGFDTFMKQSAYQFINTPQPQINPTDTNQQQADKLKALEKKI |
| | | CPFCHIQNNKIDPAKDEQIAFYVYCKLLDANHLSTLRNELIKYRASSESEL |
| | | FIHLTEIIELCLLHADNVPADYNQIYKNKEQCLQRLQPFIAENSDYTNWG |
| | | DLYVQTDDETPVVHRGIELSVKYGTAKLLEKLIASDAKFKITENEFDLWH |
| | | NARATIEAMIKKREEYHNLWLEAKEKDDKEKKTRIREKNNYARRFVET |
| | | KGEDYIDICDYIDRYNWLNNKLHFEHLKSLQNITIEMLGRMAGFVVLWE |
| | | RDFQYLDQQRVKQQGGALMDFSHGVPKYEELKPHYAYFKRLFLCNNYS |
| | | DTRNHIAHFNYLTKSAANYSLIDLINQLRELLHYDRKLKNAVAKTFIDLF |
| | | DKHGMVLKLKFDSQMHQLQVGSITSKKIFHLGTKKESDNIYTFQVPTAY |
| | | CNMCKQLLEMKN |
| 234 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | G548A- | HEKFIIDTLAASLRSVEKGELRDNGTPKETNSYHRNLKKVLQNHQALIFG |
| | N567S- | RRETPNQENRDNKQLAIYNLEVVKYLEHYFPIKSNVRKNSAADIVYYLK |
| | A737S- | SETIKSTVTHQLENAMRANLLRQGKYQHHDLKADATSENLTNLKRDEA |
| | W765N- | FVMNLITTSAFAANNIRNIIDPEQIKDILGKDDFIRSLKKDAFNKHIYGLFF |
| | D800K- | PVGAEMIEPVSLWAMRGAVQQIRNNVAHYKKDALATIFNITKFEYPGAE |
| | S807R- | NILFSNTIYKQFFEKELEQLPEAFALQLKSGGVLAQYGFDQLKFLLDVVK |
| | S819K- | FHLCRTVVPFAPGFKKVMKGGTGYQNSKQDESFYNLELTAYQLKDDFS |
| | S919K- | EEAWSARYFLLKIIYNYLFLPEFTSNDKVFADTVAWLLGKNRQQAQSSK |
| | D974Y- | NKNAYAFADIRLMESSESITDYMAYVQSQWIMEENKKEDDKKEDTRINF |
| | I1064K | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYNQIYKNKEQCLQRLQPFIAENSDYTNW |
| | | GDLYVQTDKETPVVHRGIELSVKYGTAKLLEKLIASDAKFKITENEFDLW |
| | | HNARATIEAMIKKREEYHNLWLEAKEKDDNEKKTRIREKNNYARRFVE |
| | | TKGEDYIDICDYIDRYNWLNNKLHFEHLKKLQNITIEMLGRMAGFVVLW |
| | | ERDFQYLDQQRVKQQGGALMDFSHGVPKYEELKPHYAYFKRLFLCNNY |
| | | SDTRNHIAHFNYLTKSAANYSLIDLINQLRELLHYDRKLKNAVAKTFIDL |
| | | FDKHGMVLKLKFDSQMHQLQVGSITSKKIFHLGTKKESDNIYTFQVPTA |
| | | YCNMCKQLLEMKN |
| 235 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | G548A- | HEKFIIDTLAASLRSVEKGELRDNGTPKETNSYHRNLKKVLQNHQALIFG |
| | A737S- | RRETPNQENRDNKQLAIYNLEVVKYLEHYFPIKSNVRKNSAADIVYYLK |
| | W765N- | SETIKSTVTHQLENAMRANLLRQGKYQHHDLKADATSENLTNLKRDEA |
| | S807R- | FVMNLITTSAFAANNIRNIIDPEQIKDILGKDDFIRSLKKDAFNKHIYGLFF |
| | S819K- | PVGAEMIEPVSLWAMRGAVQQIRNNVAHYKKDALATIFNITKFEYPGAE |
| | D974Y- | NILFSNTIYKQFFEKELEQLPEAFALQLKSGGVLAQYGFDQLKFLLDVVK |
| | I1064K | FHLCRTVVPFAPGFKKVMKGGTGYQNSKQDESFYNLELTAYQLKDDFS |
| | | EEAWSARYFLLKIIYNYLFLPEFTNNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYNQIYKNKEQCLQRLQPFIAENSDYTNW |
| | | GDLYVQTDDETPVVHRGIELSVKYGTAKLLEKLIASDAKFKITENEFDLW |
| | | HNARATIEAMIKKREEYHNLWLEAKEKDDNEKKTRIREKNNYARRFVE |
| | | TKGEDYIDICDYIDRYNWLNNKLHFEHLKSLQNITIEMLGRMAGFVVLW |
| | | ERDFQYLDQQRVKQQGGALMDFSHGVPKYEELKPHYAYFKRLFLCNNY |
| | | SDTRNHIAHFNYLTKSAANYSLIDLINQLRELLHYDRKLKNAVAKTFIDL |
| | | FDKHGMVLKLKFDSQMHQLQVGSITSKKIFHLGTKKESDNIYTFQVPTA |
| | | YCNMCKQLLEMKN |
| 236 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | F200Y- | FLNHKFHLPVKYQKEGQWVQFELSTCILQAVEHQNLDELQPYYDWSKW |
| | K318Q- | YINDKSGKLKKSITNNHIQIGDGESKRQKALLMWETEFTDKGELDLETY |
| | S352C- | HEKYIIDTLAASLRSVEKGELRDNGTPKETNSYHRNLKKVLQNHQALIFG |
| | V372L- | RRETPNQENRDNKQLAIYNLEVVKYLEHYFPIKSNVRKNSAADIVYYLK |
| | G548A- | SETIKSTVTHQLENAMRANLLRQGKYQHHDLKADATSENLTNLKRDEA |
| | Q585L- | FVMNLITTCAFAANNIRNIIDPEQIKDILGKDDFIRSLKKDAFNKHIYGLFF |
| | A737S- | PVGAEMIEPVSLWAMRGAVQQIRNNVAHYKKDALATIFNITKFEYPGAE |
| | S819K- | NILFSNTIYKQFFEKELEQLPEAFALQLKSGGVLAQYGFDQLKFLLDVVK |
| | D974Y- | FHLCRTVVPFAPGFKKVMKGGTGYQNSKQDESFYNLELTAYQLKDDFS |
| | I1064K | EEAWSARYFLLKIIYNYLFLPEFTNNDKVFADTVAWLLGKNRLQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDDETPVVHSGIELSVKYGTAKLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHYAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 237 | H106Q- | MRVSKVKVTVNNKDTMVLMHRSAEKGALIYDTIDKKDRTEEIIPQKKQ |
| | H139Q- | KSFELSIQNKTLLRYEAIKKQKLDRSKYKVVEDIFKYRKLPAKITLEDILP |
| | D184E- | FLNHKFQLPVKYQKEGQWVQFELSTCILQAVEHQNLDELQPYYDWSKW |
| | M311V- | YINDKSGKLKKSITNNHIQIGDGESKRQKALLMWETEFTDKGELDLETY |
| | K318Q- | HEKFIIDTLAASLRSVEKGELRDNGTPKETNSYHRNLKKVLQNHQALIFG |
| | V372L- | RRETPNQENRDNKQLAIYNLEVVKYLEHYFPIKSNVRKNSAADIVYYLK |
| | G548A- | SETIKSTVTHQLENAVRANLLRQGKYQHHDLKADATSENLTNLKRDEAF |
| | D569H- | VMNLITTSAFAANNIRNIIDPEQIKDILGKDDFIRSLKKDAFNKHIYGLFFP |
| | A737S- | VGAEMIEPVSLWAMRGAVQQIRNNVAHYKKDALATIFNITKFEYPGAEN |
| | D800K- | ILFSNTIYKQFFEKELEQLPEAFALQLKSGGVLAQYGFDQLKFLLDVVKF |
| | S807R- | HLCRTVVPFAPGFKKVMKGGTGYQNSKQDESFYNLELTAYQLKDDFSE |
| | S819K- | EAWSARYFLLKIIYNYLFLPEFTNNHKVFADTVAWLLGKNRQQAQSSKN |
| | S919K- | KNAYAFADIRLMESSESITDYMAYVQSQWIMEENKKEDDKKEDTRINFE |
| | D974Y- | KFVLQLFVKGFDTFMKQSAYQFINTPQPQINPTDTNQQQADKLKALEKKI |
| | I1064K | CPFCHIQNNKIDPAKDEQIAFYVYCKLLDANHLSTLRNELIKYRASSESEL |
| | | FIHLTEIIELCLLHADNVPADYWQIYKNKEQCLQRLQPFIAENSDYTNWG |
| | | DLYVQTDKETPVVHRGIELSVKYGTAKLLEKLIASDAKFKITENEFDLWH |
| | | NARATIEAMIKKREEYHNLWLEAKEKDDNEKKTRIREKNNYARRFVET |
| | | KGEDYIDICDYIDRYNWLNNKLHFEHLKKLQNITIEMLGRMAGFVVLWE |
| | | RDFQYLDQQRVKQQGGALMDFSHGVPKYEELKPHYAYFKRLFLCNNYS |
| | | DTRNHIAHFNYLTKSAANYSLIDLINQLRELLHYDRKLKNAVAKTFIDLF |
| | | DKHGMVLKLKFDSQMHQLQVGSITSKKIFHLGTKKESDNIYTFQVPTAY |
| | | CNMCKQLLEMKN |
| 238 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | G548A- | HEKFIIDTLAASLRSVEKGELRDNGTPKETNSYHRNLKKVLQNHQALIFG |
| | A737S- | RRETPNQENRDNKQLAIYNLEVVKYLEHYFPIKSNVRKNSAADIVYYLK |
| | D800K- | SETIKSTVTHQLENAMRANLLRQGKYQHHDLKADATSENLTNLKRDEA |
| | S819K- | FVMNLITTSAFAANNIRNIIDPEQIKDILGKDDFIRSLKKDAFNKHIYGLFF |
| | D974Y- | PVGAEMIEPVSLWAMRGAVQQIRNNVAHYKKDALATIFNITKFEYPGAE |
| | L1011V- | NILFSNTIYKQFFEKELEQLPEAFALQLKSGGVLAQYGFDQLKFLLDVVK |
| | I1064K | FHLCRTVVPFAPGFKKVMKGGTGYQNSKQDESFYNLELTAYQLKDDFS |
| | | EEAWSARYFLLKIIYNYLFLPEFTNNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDKETPVVHSGIELSVKYGTAKLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHYAYFKRLFLCN |
| | | NYSDTRNHIAHFNYLTKSAANYSLIDVINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 239 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | M311V- | FLNHKFHLPVKYQKEGQWVQFELSTCILQAVEHQNLDELQPYYDWSKW |
| | K318Q- | YINDKSGKLKKSITNNHIQIGDGESKRQKALLMWETEFTDKGELDLETY |
| | S352C- | HEKFIIDTLAASLRSVEKGELRDNGTPKETNSYHRNLKKVLQNHQALIFG |
| | V372L- | RRETPNQENRDNKQLAIYNLEVVKYLEHYFPIKSNVRKNSAADIVYYLK |
| | V501T- | SETIKSTVTHQLENAVRANLLRQGKYQHHDLKADATSENLTNLKRDEAF |
| | L537R- | VMNLITTCAFAANNIRNIIDPEQIKDILGKDDFIRSLKKDAFNKHIYGLFFP |
| | G548A- | VGAEMIEPVSLWAMRGAVQQIRNNVAHYKKDALATIFNITKFEYPGAEN |
| | A737S- | ILFSNTIYKQFFEKELEQLPEAFALQLKSGGVLAQYGFDQLKFLLDVVKF |
| | D800K- | HLCRTTVPFAPGFKKVMKGGTGYQNSKQDESFYNLELTAYQRKDDFSE |
| | S807R- | EAWSARYFLLKIIYNYLFLPEFINNDKVFADTVAWLLGKNRQQAQSSKN |
| | S819K- | KNAYAFADIRLMESSESITDYMAYVQSQWIMEENKKEDDKKEDTRINFE |
| | D974Y- | KFVLQLFVKGFDTFMKQSAYQFINTPQPQINPTDTNQQQADKLKALEKKI |
| | I1064K | CPFCHIQNNKIDPAKDEQIAFYVYCKLLDANHLSTLRNELIKYRASSESEL |
| | | FIHLTEIIELCLLHADNVPADYWQIYKNKEQCLQRLQPFIAENSDYTNWG |
| | | DLYVQTDKETPVVHRGIELSVKYGTAKLLEKLIASDAKFKITENEFDLWH |
| | | NARATIEAMIKKREEYHNLWLEAKEKDDNEKKTRIREKNNYARRFVET |
| | | KGEDYIDICDYIDRYNWLNNKLHFEHLKSLQNITIEMLGRMAGFVVLWE |
| | | RDFQYLDQQRVKQQGGALMDFSHGVPKYEELKPHYAYFKRLFLCNNYS |
| | | DTRNHIAHFNYLTKSAANYSLIDLINQLRELLHYDRKLKNAVAKTFIDLF |
| | | DKHGMVLKLKFDSQMHQLQVGSITSKKIFHLGTKKESDNIYTFQVPTAY |
| | | CNMCKQLLEMKN |
| 240 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | M311V- | FLNHKFHLPVKYQKEGQWVQFELSTCILQAVEHQNLDELQPYYDWSKW |
| | K318Q- | YINDKSGKLKKSITNNHIQIGDGESKRQKALLMWETEFTDKGELDLETY |
| | S352C- | HEKFIIDTLAASLRSVEKGELRDNGTPKETNSYHRNLKKVLQNHQALIFG |
| | V372L- | RRETPNQENRDNKQLAIYNLEVVKYLEHYFPIKSNVRKNSAADIVYYLK |
| | V492Q- | SETIKSTVTHQLENAVRANLLRQGKYQHHDLKADATSENLTNLKRDEAF |
| | G548A- | VMNLITTCAFAANNIRNIIDPEQIKDILGKDDFIRSLKKDAFNKHIYGLFFP |
| | Q585L- | VGAEMIEPVSLWAMRGAVQQIRNNVAHYKKDALATIFNITKFEYPGAEN |
| | N722R- | ILFSNTIYKQFFEKELEQLPEAFALQLKSGGVLAQYGFDQLKFLLDQVKF |
| | A737S- | HLCRTVVPFAPGFKKVMKGGTGYQNSKQDESFYNLELTAYQLKDDFSE |
| | D800K- | EAWSARYFLLKIIYNYLFLPEFTNNDKVFADTVAWLLGKNRLQAQSSKN |
| | S807R- | KNAYAFADIRLMESSESITDYMAYVQSQWIMEENKKEDDKKEDTRINFE |
| | S819K- | KFVLQLFVKGFDTFMKQSAYQFINTPQPQINPTDTNQQQADKLKALEKKI |
| | S919K- | CPFCHIQNNKIDPAKDEQIAFYVYCKLLDARHLSTLRNELIKYRASSESEL |
| | D974Y- | FIHLTEIIELCLLHADNVPADYWQIYKNKEQCLQRLQPFIAENSDYTNWG |
| | I1064K | DLYVQTDKETPVVHRGIELSVKYGTAKLLEKLIASDAKFKITENEFDLWH |
| | | NARATIEAMIKKREEYHNLWLEAKEKDDNEKKTRIREKNNYARRFVET |
| | | KGEDYIDICDYIDRYNWLNNKLHFEHLKKLQNITIEMLGRMAGFVVLWE |
| | | RDFQYLDQQRVKQQGGALMDFSHGVPKYEELKPHYAYFKRLFLCNNYS |
| | | DTRNHIAHFNYLTKSAANYSLIDLINQLRELLHYDRKLKNAVAKTFIDLF |
| | | DKHGMVLKLKFDSQMHQLQVGSITSKKIFHLGTKKESDNIYTFQVPTAY |
| | | CNMCKQLLEMKN |
| 241 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | G548A- | HEKFIIDTLAASLRSVEKGELRDNGTPKETNSYHRNLKKVLQNHQALIFG |
| | A737S- | RRETPNQENRDNKQLAIYNLEVVKYLEHYFPIKSNVRKNSAADIVYYLK |
| | W765N- | SETIKSTVTHQLENAMRANLLRQGKYQHHDLKADATSENLTNLKRDEA |
| | D800K- | FVMNLITTSAFAANNIRNIIDPEQIKDILGKDDFIRSLKKDAFNKHIYGLFF |
| | S819K- | PVGAEMIEPVSLWAMRGAVQQIRNNVAHYKKDALATIFNITKFEYPGAE |
| | D974Y- | NILFSNTIYKQFFEKELEQLPEAFALQLKSGGVLAQYGFDQLKFLLDVVK |
| | I1064K | FHLCRTVVPFAPGFKKVMKGGTGYQNSKQDESFYNLELTAYQLKDDFS |
| | | EEAWSARYFLLKIIYNYLFLPEFINNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYNQIYKNKEQCLQRLQPFIAENSDYTNW |
| | | GDLYVQTDKETPVVHSGIELSVKYGTAKLLEKLIASDAKFKITENEFDLW |
| | | HNARATIEAMIKKREEYHNLWLEAKEKDDNEKKTRIREKNNYARRFVE |
| | | TKGEDYIDICDYIDRYNWLNNKLHFEHLKSLQNITIEMLGRMAGFVVLW |
| | | ERDFQYLDQQRVKQQGGALMDFSHGVPKYEELKPHYAYFKRLFLCNNY |
| | | SDTRNHIAHFNYLTKSAANYSLIDLINQLRELLHYDRKLKNAVAKTFIDL |
| | | FDKHGMVLKLKFDSQMHQLQVGSITSKKIFHLGTKKESDNIYTFQVPTA |
| | | YCNMCKQLLEMKN |
| 242 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | V501T- | HEKFIIDTLAASLRSVEKGELRDNGTPKETNSYHRNLKKVLQNHQALIFG |
| | L537R- | RRETPNQENRDNKQLAIYNLEVVKYLEHYFPIKSNVRKNSAADIVYYLK |
| | G548A- | SETIKSTVTHQLENAMRANLLRQGKYQHHDLKADATSENLTNLKRDEA |
| | D569H- | FVMNLITTSAFAANNIRNIIDPEQIKDILGKDDFIRSLKKDAFNKHIYGLFF |
| | Q585L- | PVGAEMIEPVSLWAMRGAVQQIRNNVAHYKKDALATIFNITKFEYPGAE |
| | A737S- | NILFSNTIYKQFFEKELEQLPEAFALQLKSGGVLAQYGFDQLKFLLDVVK |
| | W765N- | FHLCRTTVPFAPGFKKVMKGGTGYQNSKQDESFYNLELTAYQRKDDFSE |
| | S807R- | EAWSARYFLLKIIYNYLFLPEFTNNHKVFADTVAWLLGKNRLQAQSSKN |
| | S819K- | KNAYAFADIRLMESSESITDYMAYVQSQWIMEENKKEDDKKEDTRINFE |
| | S919K- | KFVLQLFVKGFDTFMKQSAYQFINTPQPQINPTDTNQQQADKLKALEKKI |
| | D974Y- | CPFCHIQNNKIDPAKDEQIAFYVYCKLLDANHLSTLRNELIKYRASSESEL |
| | I1064K | FIHLTEIIELCLLHADNVPADYNQIYKNKEQCLQRLQPFIAENSDYTNWG |
| | | DLYVQTDDETPVVHRGIELSVKYGTAKLLEKLIASDAKFKITENEFDLWH |
| | | NARATIEAMIKKREEYHNLWLEAKEKDDNEKKTRIREKNNYARRFVET |
| | | KGEDYIDICDYIDRYNWLNNKLHFEHLKKLQNITIEMLGRMAGFVVLWE |
| | | RDFQYLDQQRVKQQGGALMDFSHGVPKYEELKPHYAYFKRLFLCNNYS |
| | | DTRNHIAHFNYLTKSAANYSLIDLINQLRELLHYDRKLKNAVAKTFIDLF |
| | | DKHGMVLKLKFDSQMHQLQVGSITSKKIFHLGTKKESDNIYTFQVPTAY |
| | | CNMCKQLLEMKN |
| 243 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | F200Y- | FLNHKFHLPVKYQKEGQWVQFELSTCILQAVEHQNLDELQPYYDWSKW |
| | K318Q- | YINDKSGKLKKSITNNHIQIGDGESKRQKALLMWETEFTDKGELDLETY |
| | V372L- | HEKYIIDTLAASLRSVEKGELRDNGTPKETNSYHRNLKKVLQNHQALIFG |
| | G548A- | RRETPNQENRDNKQLAIYNLEVVKYLEHYFPIKSNVRKNSAADIVYYLK |
| | A737S- | SETIKSTVTHQLENAMRANLLRQGKYQHHDLKADATSENLTNLKRDEA |
| | S819K- | FVMNLITTSAFAANNIRNIIDPEQIKDILGKDDFIRSLKKDAFNKHIYGLFF |
| | D974Y- | PVGAEMIEPVSLWAMRGAVQQIRNNVAHYKKDALATIFNITKFEYPGAE |
| | I1064K | NILFSNTIYKQFFEKELEQLPEAFALQLKSGGVLAQYGFDQLKFLLDVVK |
| | | FHLCRTVVPFAPGFKKVMKGGTGYQNSKQDESFYNLELTAYQLKDDFS |
| | | EEAWSARYFLLKIIYNYLFLPEFINNDKVFADTVAWLLGKNRQQAQSSK |
| | | NKNAYAFADIRLMESSESITDYMAYVQSQWIMEENKKEDDKKEDTRINF |
| | | EKFVLQLFVKGFDTFMKQSAYQFINTPQPQINPTDTNQQQADKLKALEK |
| | | KICPFCHIQNNKIDPAKDEQIAFYVYCKLLDANHLSTLRNELIKYRASSES |
| | | ELFIHLTEIIELCLLHADNVPADYWQIYKNKEQCLQRLQPFIAENSDYTN |
| | | WGDLYVQTDDETPVVHSGIELSVKYGTAKLLEKLIASDAKFKITENEFDL |
| | | WHNARATIEAMIKKREEYHNLWLEAKEKDDNEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHYAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 244 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | K318Q- | FLNHKFHLPVKYQKEGQWVQFELSTCILQAVEHQNLDELQPYYDWSKW |
| | V372L- | YINDKSGKLKKSITNNHIQIGDGESKRQKALLMWETEFTDKGELDLETY |
| | G548A- | HEKFIIDTLAASLRSVEKGELRDNGTPKETNSYHRNLKKVLQNHQALIFG |
| | N567S- | RRETPNQENRDNKQLAIYNLEVVKYLEHYFPIKSNVRKNSAADIVYYLK |
| | N722R- | SETIKSTVTHQLENAMRANLLRQGKYQHHDLKADATSENLTNLKRDEA |
| | A737S- | FVMNLITTSAFAANNIRNIIDPEQIKDILGKDDFIRSLKKDAFNKHIYGLFF |
| | S807R- | PVGAEMIEPVSLWAMRGAVQQIRNNVAHYKKDALATIFNITKFEYPGAE |
| | S819K- | NILFSNTIYKQFFEKELEQLPEAFALQLKSGGVLAQYGFDQLKFLLDVVK |
| | N843S- | FHLCRTVVPFAPGFKKVMKGGTGYQNSKQDESFYNLELTAYQLKDDFS |
| | N871K- | EEAWSARYFLLKIIYNYLFLPEFTSNDKVFADTVAWLLGKNRQQAQSSK |
| | D974Y- | NKNAYAFADIRLMESSESITDYMAYVQSQWIMEENKKEDDKKEDTRINF |
| | I1064K | EKFVLQLFVKGFDTFMKQSAYQFINTPQPQINPTDTNQQQADKLKALEK |
| | | KICPFCHIQNNKIDPAKDEQIAFYVYCKLLDARHLSTLRNELIKYRASSES |
| | | ELFIHLTEIIELCLLHADNVPADYWQIYKNKEQCLQRLQPFIAENSDYTN |
| | | WGDLYVQTDDETPVVHRGIELSVKYGTAKLLEKLIASDAKFKITENEFDL |
| | | WHSARATIEAMIKKREEYHNLWLEAKEKDDKEKKTRIREKNNYARRFV |
| | | ETKGEDYIDICDYIDRYNWLNNKLHFEHLKSLQNITIEMLGRMAGFVVL |
| | | WERDFQYLDQQRVKQQGGALMDFSHGVPKYEELKPHYAYFKRLFLCN |
| | | NYSDTRNHIAHFNYLTKSAANYSLIDLINQLRELLHYDRKLKNAVAKTFI |
| | | DLFDKHGMVLKLKFDSQMHQLQVGSITSKKIFHLGTKKESDNIYTFQVP |
| | | TAYCNMCKQLLEMKN |
| 245 | H106Q- | MRVSKVKVTVNNKDTMVLMHRSAEKGALIYDTIDKKDRTEEIIPQKKQ |
| | H139Q- | KSFELSIQNKTLLRYEAIKKQKLDRSKYKVVEDIFKYRKLPAKITLEDILP |
| | G154K- | FLNHKFQLPVKYQKEGQWVQFELSTCILQAVEHQNLDELQPYYDWSKW |
| | D184E- | YINDKSKKLKKSITNNHIQIGDGESKRQKALLMWETEFTDKGELDLETY |
| | M311V- | HEKFIIDTLAASLRSVEKGELRDNGTPKETNSYHRNLKKVLQNHQALIFG |
| | K318Q- | RRETPNQENRDNKQLAIYNLEVVKYLEHYFPIKSNVRKNSAADIVYYLK |
| | S352C- | SETIKSTVTHQLENAVRANLLRQGKYQHHDLKADATSENLTNLKRDEAF |
| | V372L- | VMNLITTCAFAANNIRNIIDPEQIKDILGKDDFIRSLKKDAFNKHIYGLFFP |
| | V501T- | VGAEMIEPVSLWAMRGAVQQIRNNVAHYKKDALATIFNITKFEYPGAEN |
| | G548A- | ILFSNTIYKQFFEKELEQLPEAFALQLKSGGVLAQYGFDQLKFLLDVVKF |
| | W621Q- | HLCRTTVPFAPGFKKVMKGGTGYQNSKQDESFYNLELTAYQLKDDFSEE |
| | N722R- | AWSARYFLLKIIYNYLFLPEFTNNDKVFADTVAWLLGKNRQQAQSSKNK |
| | A737S- | NAYAFADIRLMESSESITDYMAYVQSQQIMEENKKEDDKKEDTRINFEK |
| | A762G- | FVLQLFVKGFDTFMKQSAYQFINTPQPQINPTDTNQQQADKLKALEKKIC |
| | W765N- | PFCHIQNNKIDPAKDEQIAFYVYCKLLDARHLSTLRNELIKYRASSESELFI |
| | S807R- | HLTEIIELCLLHADNVPGDYNQIYKNKEQCLQRLQPFIAENSDYTNWGDL |
| | S819K- | YVQTDDETPVVHRGIELSVKYGTAKLLEKLIAKDAKFKITENEFDLWHN |
| | S827K- | ARATIEAMIKKREEYHNLWLEAKEKDDKEKKTRIREKNNYARRFVETK |
| | N871K- | GEDYIDICDYIDRYNWLNNKLHFEHLKSLQNITIEMLGRMAGFVVLWER |
| | D974Y- | DFQYLDQQRVKQQGGALMDFSHGIPKYEELKPHYAYFKRLFLCNNYSD |
| | I1064K | TRNHIAHFNYLTKSAANYSLIDLINQLRELLHYDRKLKNAVAKTFIDLFD |
| | | KHGMVLKLKFDSQMHQLQVGSITSKKIFHLGTKKESDNIYTFQVPTAYC |
| | | NMCKQLLEMKN |
| 246 | H139Q- | MRVSKVKVTVNNKDTMVLMHRSAEKGALIYDTIDKKDRTEEIIPQKKQ |
| | G154K- | KSFELSIQNKTLLRYEAIKKQKLDRSKYKVVEDIFKYRKLPAKITLEDILP |
| | D184E- | FLNHKFHLPVKYQKEGQWVQFELSTCILQAVEHQNLDELQPYYDWSKW |
| | M311V- | YINDKSKKLKKSITNNHIQIGDGESKRQKALLMWETEFTDKGELDLETY |
| | K318Q- | HEKFIIDTLAASLRSVEKGELRDNGTPKETNSYHRNLKKVLQNHQALIFG |
| | S352C- | RRETPNQENRDNKQLAIYNLEVVKYLEHYFPIKSNVRKNSAADIVYYLK |
| | V372L- | SETIKSTVTHQLENAVRANLLRQGKYQHHDLKADATSENLTNLKRDEAF |
| | V501T- | VMNLITTCAFAANNIRNIIDPEQIKDILGKDDFIRSLKKDAFNKHIYGLFFP |
| | G548A- | VGAEMIEPVSLWAMRGAVQQIRNNVAHYKKDALATIFNITKFEYPGAEN |
| | T726Q- | ILFSNTIYKQFFEKELEQLPEAFALQLKSGGVLAQYGFDQLKFLLDVVKF |
| | N722R- | HLCRTTVPFAPGFKKVMKGGTGYQNSKQDESFYNLELTAYQLKDDFSEE |
| | A737S- | AWSARYFLLKIIYNYLFLPEFTNNDKVFADTVAWLLGKNRQQAQSSKNK |
| | W765N- | NAYAFADIRLMESSESITDYMAYVQSQWIMEENKKEDDKKEDTRINFEK |
| | S807R- | FVLQLFVKGFDTFMKQSAYQFINTPQPQINPTDTNQQQADKLKALEKKIC |
| | S819K- | PFCHIQNNKIDPAKDEQIAFYVYCKLLDARHLSQLRNELIKYRASSESELF |
| | N871K- | IHLTEIIELCLLHADNVPADYNQIYKNKEQCLQRLQPFIAENSDYTNWGD |
| | D974Y- | LYVQTDDETPVVHRGIELSVKYGTAKLLEKLIASDAKFKITENEFDLWHN |
| | H1020R- | ARATIEAMIKKREEYHNLWLEAKEKDDKEKKTRIREKNNYARRFVETK |
| | I1064K | GEDYIDICDYIDRYNWLNNKLHFEHLKSLQNITIEMLGRMAGFVVLWER |
| | | DFQYLDQQRVKQQGGALMDFSHGVPKYEELKPHYAYFKRLFLCNNYSD |
| | | TRNHIAHFNYLTKSAANYSLIDLINQLRELLRYDRKLKNAVAKTFIDLFD |
| | | KHGMVLKLKFDSQMHQLQVGSITSKKIFHLGTKKESDNIYTFQVPTAYC |
| | | NMCKQLLEMKN |
| 247 | H106Q- | MRVSKVKVTVNNKDTMVLMHRSAEKGALIYDTIDKKDRTEEIIPQKKQ |
| | H139Q- | KSFELSIQNKTLLRYEAIKKQKLDRSKYKVVEDIFKYRKLPAKITLEDILP |
| | G154K- | FLNHKFQLPVKYQKEGQWVQFELSTCILQAVEHQNLDELQPYYDWSKW |
| | D184E- | YINDKSKKLKKSITNNHIQIGDGESKRQKALLMWETEFTDKGELDLETY |
| | M311V- | HEKFIIDTLAASLRSVEKGELRDNGTPKETNSYHRNLKKVLQNHQALIFG |
| | K318Q- | RRETPNQENRDNKQLAIYNLEVVKYLEHYFPIKSNVRKNSAADIVYYLK |
| | S352C- | SETIKSTVTHQLENAVRANLLRQGKYQHHDLKADATSENLTNLKRDEAF |
| | V372L- | VMNLITTCAFAANNIRNIIDPEQIKDILGKDDFIRSLKKDAFNKHIYGLFFP |
| | V501T- | VGAEMIEPVSLWAMRGAVQQIRNNVAHYKKDALATIFNITKFEYPGAEN |
| | G548A- | ILFSNTIYKQFFEKELEQLPEAFALQLKSGGVLAQYGFDQLKFLLDVVKF |
| | N722R- | HLCRTTVPFAPGFKKVMKGGTGYQNSKQDESFYNLELTAYQLKDDFSEE |
| | A737S- | AWSARYFLLKIIYNYLFLPEFTNNDKVFADTVAWLLGKNRQQAQSSKNK |
| | A762G- | NAYAFADIRLMESSESITDYMAYVQSQWIMEENKKEDDKKEDTRINFEK |
| | W765N- | FVLQLFVKGFDTFMKQSAYQFINTPQPQINPTDTNQQQADKLKALEKKIC |
| | S807R- | PFCHIQNNKIDPAKDEQIAFYVYCKLLDARHLSTLRNELIKYRASSESELFI |
| | S819K- | HLTEIIELCLLHADNVPGDYNQIYKNKEQCLQRLQPFIAENSDYTNWGDL |
| | N871K- | YVQTDDETPVVHRGIELSVKYGTAKLLEKLIASDAKFKITENEFDLWHN |
| | D974Y- | ARATIEAMIKKREEYHNLWLEAKEKDDKEKKTRIREKNNYARRFVETK |
| | D1049G- | GEDYIDICDYIDRYNWLNNKLHFEHLKSLQNITIEMLGRMAGFVVLWER |
| | I1064K | DFQYLDQQRVKQQGGALMDFSHGVPKYEELKPHYAYFKRLFLCNNYSD |
| | | TRNHIAHFNYLTKSAANYSLIDLINQLRELLHYDRKLKNAVAKTFIDLFD |
| | | KHGMVLKLKFGSQMHQLQVGSITSKKIFHLGTKKESDNIYTFQVPTAYC |
| | | NMCKQLLEMKN |
| 248 | H106Q- | MRVSKVKVTVNNKDTMVLMHRSAEKGALIYDTIDKKDRTEEIIPQKKQ |
| | H139Q- | KSFELSIQNKTLLRYEAIKKQKLDRSKYKVVEDIFKYRKLPAKITLEDILP |
| | D184E- | FLNHKFQLPVKYQKEGQWVQFELSTCILQAVEHQNLDELQPYYDWSKW |
| | M311V- | YINDKSGKLKKSITNNHIQIGDGESKRQKALLMWETEFTDKGELDLETY |
| | K318Q- | HEKFIIDTLAASLRSVEKGELRDNGTPKETNSYHRNLKKVLQNHQALIFG |
| | S352C- | RRETPNQENRDNKQLAIYNLEVVKYLEHYFPIKSNVRKNSAADIVYYLK |
| | V372L- | SETIKSTVTHQLENAVRANLLRQGKYQHHDLKADATSENLTNLKRDEAF |
| | V501T- | VMNLITTCAFAANNIRNIIDPEQIKDILGKDDFIRSLKKDAFNKHIYGLFFP |
| | G548A- | VGAEMIEPVSLWAMRGAVQQIRNNVAHYKKDALATIFNITKFEYPGAEN |
| | I622N- | ILFSNTIYKQFFEKELEQLPEAFALQLKSGGVLAQYGFDQLKFLLDVVKF |
| | N722R- | HLCRTTVPFAPGFKKVMKGGTGYQNSKQDESFYNLELTAYQLKDDFSEE |
| | A737S- | AWSARYFLLKIIYNYLFLPEFTNNDKVFADTVAWLLGKNRQQAQSSKNK |
| | W765N- | NAYAFADIRLMESSESITDYMAYVQSQWNMEENKKEDDKKEDTRINFE |
| | S807R- | KFVLQLFVKGFDTFMKQSAYQFINTPQPQINPTDTNQQQADKLKALEKKI |
| | S819K- | CPFCHIQNNKIDPAKDEQIAFYVYCKLLDARHLSTLRNELIKYRASSESEL |
| | S827K- | FIHLTEIIELCLLHADNVPADYNQIYKNKEQCLQRLQPFIAENSDYTNWG |
| | N871K- | DLYVQTDDETPVVHRGIELSVKYGTAKLLEKLIAKDAKFKITENEFDLW |
| | D974Y- | HNARATIEAMIKKREEYHNLWLEAKEKDDKEKKTRIREKNNYARRFVE |
| | H1020R- | TKGEDYIDICDYIDRYNWLNNKLHFEHLKSLQNITIEMLGRMAGFVVLW |
| | I1064K | ERDFQYLDQQRVKQQGGALMDFSHGVPKYEELKPHYAYFKRLFLCNNY |
| | | SDTRNHIAHFNYLTKSAANYSLIDLINQLRELLRYDRKLKNAVAKTFIDL |
| | | FDKHGMVLKLKFDSQMHQLQVGSITSKKIFHLGTKKESDNIYTFQVPTA |
| | | YCNMCKQLLEMKN |
| 249 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | A262T- | FLNHKFHLPVKYQKEGQWVQFELSTCILQAVEHQNLDELQPYYDWSKW |
| | M311V- | YINDKSGKLKKSITNNHIQIGDGESKRQKALLMWETEFTDKGELDLETY |
| | K318Q- | HEKFIIDTLAASLRSVEKGELRDNGTPKETNSYHRNLKKVLQNHQALIFG |
| | S352C- | RRETPNQENRDNKQLTIYNLEVVKYLEHYFPIKSNVRKNSAADIVYYLKS |
| | V372L- | ETIKSTVTHQLENAVRANLLRQGKYQHHDLKADATSENLTNLKRDEAF |
| | V501T- | VMNLITTCAFAANNIRNIIDPEQIKDILGKDDFIRSLKKDAFNKHIYGLFFP |
| | E543L- | VGAEMIEPVSLWAMRGAVQQIRNNVAHYKKDALATIFNITKFEYPGAEN |
| | G548A- | ILFSNTIYKQFFEKELEQLPEAFALQLKSGGVLAQYGFDQLKFLLDVVKF |
| | I622N- | HLCRTTVPFAPGFKKVMKGGTGYQNSKQDESFYNLELTAYQLKDDFSLE |
| | N722R- | AWSARYFLLKIIYNYLFLPEFTNNDKVFADTVAWLLGKNRQQAQSSKNK |
| | A737S- | NAYAFADIRLMESSESITDYMAYVQSQWNMEENKKEDDKKEDTRINFE |
| | W765N- | KFVLQLFVKGFDTFMKQSAYQFINTPQPQINPTDTNQQQADKLKALEKKI |
| | D800R- | CPFCHIQNNKIDPAKDEQIAFYVYCKLLDARHLSTLRNELIKYRASSESEL |
| | S807R- | FIHLTEIIELCLLHADNVPADYNQIYKNKEQCLQRLQPFIAENSDYTNWG |
| | S819K- | DLYVQTDRETPVVHRGIELSVKYGTAKLLEKLIAKDAKFKITENEFDLW |
| | S827K- | HNARATIEAMIKKREEYHNLWLEAKEKDDKEKKTRIREKNNYARRFVE |
| | N871K- | TKGEDYIDICDYIDRYNWLNNKLHFEHLKSLQNITIEMLGRMAGFVVLW |
| | D974Y- | ERDFQYLDQQRVKQQGGALMDFSHGVPKYEELKPHYAYFKRLFLCNNY |
| | H1020R- | SDTRNHIAHFNYLTKSAANYSLIDLINQLRELLRYDRKLKNAVAKTFIDL |
| | I1064K | FDKHGMVLKLKFDSQMHQLQVGSITSKKIFHLGTKKESDNIYTFQVPTA |
| | | YCNMCKQLLEMKN |
| 250 | H106Q- | MRVSKVKVTVNNKDTMVLMHRSAEKGALIYDTIDKKDRTEEIIPQKKQ |
| | H139Q- | KSFELSIQNKTLLRYEAIKKQKLDRSKYKVVEDIFKYRKLPAKITLEDILP |
| | G154K- | FLNHKFQLPVKYQKEGQWVQFELSTCILQAVEHQNLDELQPYYDWSKW |
| | D184E- | YINDKSKKLKKSITNNHIQIGDGESKRQKALLMWETEFTDKGELDLETY |
| | M311V- | HEKFIIDTLAASLRSVEKGELRDNGTPKETNSYHRNLKKVLQNHQALIFG |
| | K318Q- | RRETPNQENRDNKQLAIYNLEVVKYLEHYFPIKSNVRKNSAADIVYYLK |
| | S352C- | SETIKSTVTHQLENAVRANLLRQGKYQHHDLKADATSENLTNLKRDEAF |
| | V372L- | VMNLITTCAFAANNIRNIIDPEQIKDILGKDDFIRSLKKDAFNKHIYGLFFP |
| | V501T- | VGAEMIEPVSLWAMRGAVQQIRNNVAHYKKDALATIFNITKFEYPGAEN |
| | G548A- | ILFSNTIYKQFFEKELEQLPEAFALQLKSGGVLAQYGFDQLKFLLDVVKF |
| | L684K- | HLCRTTVPFAPGFKKVMKGGTGYQNSKQDESFYNLELTAYQLKDDFSEE |
| | N722R- | AWSARYFLLKIIYNYLFLPEFINNDKVFADTVAWLLGKNRQQAQSSKNK |
| | A737S- | NAYAFADIRLMESSESITDYMAYVQSQWIMEENKKEDDKKEDTRINFEK |
| | W765N- | FVLQLFVKGFDTFMKQSAYQFINTPQPQINPTDTNQQQADKKKALEKKI |
| | S807R- | CPFCHIQNNKIDPAKDEQIAFYVYCKLLDARHLSTLRNELIKYRASSESEL |
| | S819K- | FIHLTEIIELCLLHADNVPADYNQIYKNKEQCLQRLQPFIAENSDYTNWG |
| | S827K- | DLYVQTDDETPVVHRGIELSVKYGTAKLLEKLIAKDAKFKITENEFDLW |
| | N871K- | HNARATIEAMIKKREEYHNLWLEAKEKDDKEKKTRIREKNNYARRFVE |
| | D974Y- | TKGEDYIDICDYIDRYNWLNNKLHFEHLKSLQNITIEMLGRMAGFVVLW |
| | H1020R- | ERDFQYLDQQRVKQQGGALMDFSHGVPKYEELKPHYAYFKRLFLCNNY |
| | I1064K | SDTRNHIAHFNYLTKSAANYSLIDLINQLRELLRYDRKLKNAVAKTFIDL |
| | | FDKHGMVLKLKFDSQMHQLQVGSITSKKIFHLGTKKESDNIYTFQVPTA |
| | | YCNMCKQLLEMKN |
| 251 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | M311V- | FLNHKFHLPVKYQKEGQWVQFELSTCILQAVEHQNLDELQPYYDWSKW |
| | K318Q- | YINDKSGKLKKSITNNHIQIGDGESKRQKALLMWETEFTDKGELDLETY |
| | S352C- | HEKFIIDTLAASLRSVEKGELRDNGTPKETNSYHRNLKKVLQNHQALIFG |
| | V372L- | RRETPNQENRDNKQLAIYNLEVVKYLEHYFPIKSNVRKNSAADIVYYLK |
| | V501T- | SETIKSTVTHQLENAVRANLLRQGKYQHHDLKADATSENLTNLKRDEAF |
| | G548A- | VMNLITTCAFAANNIRNIIDPEQIKDILGKDDFIRSLKKDAFNKHIYGLFFP |
| | N722R- | VGAEMIEPVSLWAMRGAVQQIRNNVAHYKKDALATIFNITKFEYPGAEN |
| | A737S- | ILFSNTIYKQFFEKELEQLPEAFALQLKSGGVLAQYGFDQLKFLLDVVKF |
| | A762G- | HLCRTTVPFAPGFKKVMKGGTGYQNSKQDESFYNLELTAYQLKDDFSEE |
| | W765N- | AWSARYFLLKIIYNYLFLPEFTNNDKVFADTVAWLLGKNRQQAQSSKNK |
| | S807R- | NAYAFADIRLMESSESITDYMAYVQSQWIMEENKKEDDKKEDTRINFEK |
| | S819K- | FVLQLFVKGFDTFMKQSAYQFINTPQPQINPTDTNQQQADKLKALEKKIC |
| | S827K- | PFCHIQNNKIDPAKDEQIAFYVYCKLLDARHLSTLRNELIKYRASSESELFI |
| | N871K- | HLTEIIELCLLHADNVPGDYNQIYKNKEQCLQRLQPFIAENSDYTNWGDL |
| | D974Y- | YVQTDDETPVVHRGIELSVKYGTAKLLEKLIAKDAKFKITENEFDLWHN |
| | I1064K | ARATIEAMIKKREEYHNLWLEAKEKDDKEKKTRIREKNNYARRFVETK |
| | | GEDYIDICDYIDRYNWLNNKLHFEHLKSLQNITIEMLGRMAGFVVLWER |
| | | DFQYLDQQRVKQQGGALMDFSHGVPKYEELKPHYAYFKRLFLCNNYSD |
| | | TRNHIAHFNYLTKSAANYSLIDLINQLRELLHYDRKLKNAVAKTFIDLFD |
| | | KHGMVLKLKFDSQMHQLQVGSITSKKIFHLGTKKESDNIYTFQVPTAYC |
| | | NMCKQLLEMKN |
| 252 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | M311V- | FLNHKFHLPVKYQKEGQWVQFELSTCILQAVEHQNLDELQPYYDWSKW |
| | K318Q- | YINDKSGKLKKSITNNHIQIGDGESKRQKALLMWETEFTDKGELDLETY |
| | S352C- | HEKFIIDTLAASLRSVEKGELRDNGTPKETNSYHRNLKKVLQNHQALIFG |
| | V372L- | RRETPNQENRDNKQLAIYNLEVVKYLEHYFPIKSNVRKNSAADIVYYLK |
| | V501T- | SETIKSTVTHQLENAVRANLLRQGKYQHHDLKADATSENLTNLKRDEAF |
| | E543L- | VMNLITTCAFAANNIRNIIDPEQIKDILGKDDFIRSLKKDAFNKHIYGLFFP |
| | G548A- | VGAEMIEPVSLWAMRGAVQQIRNNVAHYKKDALATIFNITKFEYPGAEN |
| | I622N- | ILFSNTIYKQFFEKELEQLPEAFALQLKSGGVLAQYGFDQLKFLLDVVKF |
| | N722R- | HLCRTTVPFAPGFKKVMKGGTGYQNSKQDESFYNLELTAYQLKDDFSLE |
| | A737S- | AWSARYFLLKIIYNYLFLPEFTNNDKVFADTVAWLLGKNRQQAQSSKNK |
| | W765N- | NAYAFADIRLMESSESITDYMAYVQSQWNMEENKKEDDKKEDTRINFE |
| | D800R- | KFVLQLFVKGFDTFMKQSAYQFINTPQPQINPTDTNQQQADKLKALEKKI |
| | S807R- | CPFCHIQNNKIDPAKDEQIAFYVYCKLLDARHLSTLRNELIKYRASSESEL |
| | S819K- | FIHLTEIIELCLLHADNVPADYNQIYKNKEQCLQRLQPFIAENSDYTNWG |
| | S827K- | DLYVQTDRETPVVHRGIELSVKYGTAKLLEKLIAKDAKFKITENEFDLW |
| | N871K- | HNARATIEAMIKKREEYHNLWLEAKEKDDKEKKTRIREKNNYARRFVE |
| | D974Y- | TKGEDYIDICDYIDRYNWLNNKLHFEHLKSLQNITIEMLGRMAGFVVLW |
| | I1064K | ERDFQYLDQQRVKQQGGALMDFSHGVPKYEELKPHYAYFKRLFLCNNY |
| | | SDTRNHIAHFNYLTKSAANYSLIDLINQLRELLHYDRKLKNAVAKTFIDL |
| | | FDKHGMVLKLKFDSQMHQLQVGSITSKKIFHLGTKKESDNIYTFQVPTA |
| | | YCNMCKQLLEMKN |
| 253 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | M311V- | FLNHKFHLPVKYQKEGQWVQFELSTCILQAVEHQNLDELQPYYDWSKW |
| | K318Q- | YINDKSGKLKKSITNNHIQIGDGESKRQKALLMWETEFTDKGELDLETY |
| | S352C- | HEKFIIDTLAASLRSVEKGELRDNGTPKETNSYHRNLKKVLQNHQALIFG |
| | V372L- | RRETPNQENRDNKQLAIYNLEVVKYLEHYFPIKSNVRKNSAADIVYYLK |
| | V501T- | SETIKSTVTHQLENAVRANLLRQGKYQHHDLKADATSENLTNLKRDEAF |
| | E543L- | VMNLITTCAFAANNIRNIIDPEQIKDILGKDDFIRSLKKDAFNKHIYGLFFP |
| | G548A- | VGAEMIEPVSLWAMRGAVQQIRNNVAHYKKDALATIFNITKFEYPGAEN |
| | N722R- | ILFSNTIYKQFFEKELEQLPEAFALQLKSGGVLAQYGFDQLKFLLDVVKF |
| | A737S- | HLCRTTVPFAPGFKKVMKGGTGYQNSKQDESFYNLELTAYQLKDDFSLE |
| | W765N- | AWSARYFLLKIIYNYLFLPEFTNNDKVFADTVAWLLGKNRQQAQSSKNK |
| | S807R- | NAYAFADIRLMESSESITDYMAYVQSQWIMEENKKEDDKKEDTRINFEK |
| | S819K- | FVLQLFVKGFDTFMKQSAYQFINTPQPQINPTDTNQQQADKLKALEKKIC |
| | S827K- | PFCHIQNNKIDPAKDEQIAFYVYCKLLDARHLSTLRNELIKYRASSESELFI |
| | N871K- | HLTEIIELCLLHADNVPADYNQIYKNKEQCLQRLQPFIAENSDYTNWGDL |
| | D974Y- | YVQTDDETPVVHRGIELSVKYGTAKLLEKLIAKDAKFKITENEFDLWHN |
| | I1064K | ARATIEAMIKKREEYHNLWLEAKEKDDKEKKTRIREKNNYARRFVETK |
| | | GEDYIDICDYIDRYNWLNNKLHFEHLKSLQNITIEMLGRMAGFVVLWER |
| | | DFQYLDQQRVKQQGGALMDFSHGVPKYEELKPHYAYFKRLFLCNNYSD |
| | | TRNHIAHFNYLTKSAANYSLIDLINQLRELLHYDRKLKNAVAKTFIDLFD |
| | | KHGMVLKLKFDSQMHQLQVGSITSKKIFHLGTKKESDNIYTFQVPTAYC |
| | | NMCKQLLEMKN |
| 254 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | M311V- | FLNHKFHLPVKYQKEGQWVQFELSTCILQAVEHQNLDELQPYYDWSKW |
| | K318Q- | YINDKSGKLKKSITNNHIQIGDGESKRQKALLMWETEFTDKGELDLETY |
| | S352C- | HEKFIIDTLAASLRSVEKGELRDNGTPKETNSYHRNLKKVLQNHQALIFG |
| | V372L- | RRETPNQENRDNKQLAIYNLEVVKYLEHYFPIKSNVRKNSAADIVYYLK |
| | V501T- | SETIKSTVTHQLENAVRANLLRQGKYQHHDLKADATSENLTNLKRDEAF |
| | G548A- | VMNLITTCAFAANNIRNIIDPEQIKDILGKDDFIRSLKKDAFNKHIYGLFFP |
| | I622N- | VGAEMIEPVSLWAMRGAVQQIRNNVAHYKKDALATIFNITKFEYPGAEN |
| | N722R- | ILFSNTIYKQFFEKELEQLPEAFALQLKSGGVLAQYGFDQLKFLLDVVKF |
| | A737S- | HLCRTTVPFAPGFKKVMKGGTGYQNSKQDESFYNLELTAYQLKDDFSEE |
| | W765N- | AWSARYFLLKIIYNYLFLPEFTNNDKVFADTVAWLLGKNRQQAQSSKNK |
| | S807R- | NAYAFADIRLMESSESITDYMAYVQSQWNMEENKKEDDKKEDTRINFE |
| | S819K- | KFVLQLFVKGFDTFMKQSAYQFINTPQPQINPTDTNQQQADKLKALEKKI |
| | N871K- | CPFCHIQNNKIDPAKDEQIAFYVYCKLLDARHLSTLRNELIKYRASSESEL |
| | D974Y- | FIHLTEIIELCLLHADNVPADYNQIYKNKEQCLQRLQPFIAENSDYTNWG |
| | I1064K | DLYVQTDDETPVVHRGIELSVKYGTAKLLEKLIASDAKFKITENEFDLWH |
| | | NARATIEAMIKKREEYHNLWLEAKEKDDKEKKTRIREKNNYARRFVET |
| | | KGEDYIDICDYIDRYNWLNNKLHFEHLKSLQNITIEMLGRMAGFVVLWE |
| | | RDFQYLDQQRVKQQGGALMDFSHGVPKYEELKPHYAYFKRLFLCNNYS |
| | | DTRNHIAHFNYLTKSAANYSLIDLINQLRELLHYDRKLKNAVAKTFIDLF |
| | | DKHGMVLKLKFDSQMHQLQVGSITSKKIFHLGTKKESDNIYTFQVPTAY |
| | | CNMCKQLLEMKN |
| 255 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | M311V- | FLNHKFHLPVKYQKEGQWVQFELSTCILQAVEHQNLDELQPYYDWSKW |
| | K318Q- | YINDKSGKLKKSITNNHIQIGDGESKRQKALLMWETEFTDKGELDLETY |
| | S352C- | HEKFIIDTLAASLRSVEKGELRDNGTPKETNSYHRNLKKVLQNHQALIFG |
| | V372L- | RRETPNQENRDNKQLAIYNLEVVKYLEHYFPIKSNLRKNSAADIVYYLKS |
| | V501T- | ETIKSTVTHQLENAVRANLLRQGKYQHHDLKADATSENLTNLKRDEAF |
| | E543L- | VMNLITTCAFAANNIRNIIDPEQIKDILGKDDFIRSLKKDAFNKHIYGLFFP |
| | G548A- | VGAEMIEPVSLWAMRGAVQQIRNNVAHYKKDALATIFNITKFEYPGAEN |
| | N722R- | ILFSNTIYKQFFEKELEQLPEAFALQLKSGGVLAQYGFDQLKFLLDVVKF |
| | A737S- | HLCRTTVPFAPGFKKVMKGGTGYQNSKQDESFYNLELTAYQLKDDFSLE |
| | W765N- | AWSARYFLLKIIYNYLFLPEFTNNDKVFADTVAWLLGKNRQQAQSSKNK |
| | S807R- | NAYAFADIRLMESSESITDYMAYVQSQWIMEENKKEDDKKEDTRINFEK |
| | S819K- | FVLQLFVKGFDTFMKQSAYQFINTPQPQINPTDTNQQQADKLKALEKKIC |
| | S827K- | PFCHIQNNKIDPAKDEQIAFYVYCKLLDARHLSTLRNELIKYRASSESELFI |
| | N871K- | HLTEIIELCLLHADNVPADYNQIYKNKEQCLQRLQPFIAENSDYTNWGDL |
| | D974Y- | YVQTDDETPVVHRGIELSVKYGTAKLLEKLIAKDAKFKITENEFDLWHN |
| | T1032V- | ARATIEAMIKKREEYHNLWLEAKEKDDKEKKTRIREKNNYARRFVETK |
| | I1064K | GEDYIDICDYIDRYNWLNNKLHFEHLKSLQNITIEMLGRMAGFVVLWER |
| | | DFQYLDQQRVKQQGGALMDFSHGVPKYEELKPHYAYFKRLFLCNNYSD |
| | | TRNHIAHFNYLTKSAANYSLIDLINQLRELLHYDRKLKNAVAKVFIDLFD |
| | | KHGMVLKLKFDSQMHQLQVGSITSKKIFHLGTKKESDNIYTFQVPTAYC |
| | | NMCKQLLEMKN |
| 256 | H106Q- | MRVSKVKVTVNNKDTMVLMHRSAEKGALIYDTIDKKDRTEEIIPQKKQ |
| | H139Q- | KSFELSIQNKTLLRYEAIKKQKLDRSKYKVVEDIFKYRKLPAKITLEDILP |
| | G154K- | FLNHKFQLPVKYQKEGQWVQFELSTCILQAVEHQNLDELQPYYDWSKW |
| | D184E- | YINDKSKKLKKSITNNHIQIGDGESKRQKALLMWETEFTDKGELDLETY |
| | M311V- | HEKFIIDTLAASLRSVEKGELRDNGTPKETNSYHRNLKKVLQNHQALIFG |
| | K318Q- | RRETPNQENRDNKQLAIYNLEVVKYLEHYFPIKSNVRKNSAADIVYYLK |
| | S352C- | SETIKSTVTHQLENAVRANLLRQGKYQHHDLKADATSENLTNLKRDEAF |
| | V372L- | VMNLITTCAFAANNIRNIIDPEQIKDILGKDDFIRSLKKDAFNKHIYGLFFP |
| | V501T- | VGAEMIEPVSLWAMRGAVQQIRNNVAHYKKDALATIFNITKFEYPGAEN |
| | G548A- | ILFSNTIYKQFFEKELEQLPEAFALQLKSGGVLAQYGFDQLKFLLDVVKF |
| | I622N- | HLCRTTVPFAPGFKKVMKGGTGYQNSKQDESFYNLELTAYQLKDDFSEE |
| | T726Q- | AWSARYFLLKIIYNYLFLPEFINNDKVFADTVAWLLGKNRQQAQSSKNK |
| | N722R- | NAYAFADIRLMESSESITDYMAYVQSQWNMEENKKEDDKKEDTRINFE |
| | A737S- | KFVLQLFVKGFDTFMKQSAYQFINTPQPQINPTDTNQQQADKLKALEKKI |
| | W765N- | CPFCHIQNNKIDPAKDEQIAFYVYCKLLDARHLSQLRNELIKYRASSESEL |
| | S807R- | FIHLTEIIELCLLHADNVPADYNQIYKNKEQCLQRLQPFIAENSDYTNWG |
| | S819K- | DLYVQTDDETPVVHRGIELSVKYGTAKLLEKLIAKDAKFKITENEFDLW |
| | S827K- | HNARATIEAMIKKREEYHNLWLEAKEKDDKEKKTRIREKNNYARRFVE |
| | N871K- | TKGEDYIDICDYIDRYNWLNNKLHFEHLKSLQNITIEMLGRMAGFVVLW |
| | D974Y- | ERDFQYLDQQRVKQQGGALMDFSHGVPKYEELKPHYAYFKRLFLCNNY |
| | D1049G- | SDTRNHIAHFNYLTKSAANYSLIDLINQLRELLHYDRKLKNAVAKTFIDL |
| | I1064K | FDKHGMVLKLKFGSQMHQLQVGSITSKKIFHLGTKKESDNIYTFQVPTA |
| | | YCNMCKQLLEMKN |
| 257 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | M311V- | FLNHKFHLPVKYQKEGQWVQFELSTCILQAVEHQNLDELQPYYDWSKW |
| | K318Q- | YINDKSGKLKKSITNNHIQIGDGESKRQKALLMWETEFTDKGELDLETY |
| | S352C- | HEKFIIDTLAASLRSVEKGELRDNGTPKETNSYHRNLKKVLQNHQALIFG |
| | V372L- | RRETPNQENRDNKQLAIYNLEVVKYLEHYFPIKSNVRKNSAADIVYYLK |
| | V501T- | SETIKSTVTHQLENAVRANLLRQGKYQHHDLKADATSENLTNLKRDEAF |
| | G548A- | VMNLITTCAFAANNIRNIIDPEQIKDILGKDDFIRSLKKDAFNKHIYGLFFP |
| | N722R- | VGAEMIEPVSLWAMRGAVQQIRNNVAHYKKDALATIFNITKFEYPGAEN |
| | A737S- | ILFSNTIYKQFFEKELEQLPEAFALQLKSGGVLAQYGFDQLKFLLDVVKF |
| | W765N- | HLCRTTVPFAPGFKKVMKGGTGYQNSKQDESFYNLELTAYQLKDDFSEE |
| | S807R- | AWSARYFLLKIIYNYLFLPEFINNDKVFADTVAWLLGKNRQQAQSSKNK |
| | S819K- | NAYAFADIRLMESSESITDYMAYVQSQWIMEENKKEDDKKEDTRINFEK |
| | S827K- | FVLQLFVKGFDTFMKQSAYQFINTPQPQINPTDTNQQQADKLKALEKKIC |
| | N871K- | PFCHIQNNKIDPAKDEQIAFYVYCKLLDARHLSTLRNELIKYRASSESELFI |
| | D974Y- | HLTEIIELCLLHADNVPADYNQIYKNKEQCLQRLQPFIAENSDYTNWGDL |
| | I1064K | YVQTDDETPVVHRGIELSVKYGTAKLLEKLIAKDAKFKITENEFDLWHN |
| | | ARATIEAMIKKREEYHNLWLEAKEKDDKEKKTRIREKNNYARRFVETK |
| | | GEDYIDICDYIDRYNWLNNKLHFEHLKSLQNITIEMLGRMAGFVVLWER |
| | | DFQYLDQQRVKQQGGALMDFSHGVPKYEELKPHYAYFKRLFLCNNYSD |
| | | TRNHIAHFNYLTKSAANYSLIDLINQLRELLHYDRKLKNAVAKTFIDLFD |
| | | KHGMVLKLKFDSQMHQLQVGSITSKKIFHLGTKKESDNIYTFQVPTAYC |
| | | NMCKQLLEMKN |
| 258 | H106Q- | MRVSKVKVTVNNKDTMVLMHRSAEKGALIYDTIDKKDRTEEIIPQKKQ |
| | H139Q- | KSFELSIQNKTLLRYEAIKKQKLDRSKYKVVEDIFKYRKLPAKITLEDILP |
| | D184E- | FLNHKFQLPVKYQKEGQWVQFELSTCILQAVEHQNLDELQPYYDWSKW |
| | A262T- | YINDKSGKLKKSITNNHIQIGDGESKRQKALLMWETEFTDKGELDLETY |
| | M311V- | HEKFIIDTLAASLRSVEKGELRDNGTPKETNSYHRNLKKVLQNHQALIFG |
| | K318Q- | RRETPNQENRDNKQLTIYNLEVVKYLEHYFPIKSNVRKNSAADIVYYLKS |
| | S352C- | ETIKSTVTHQLENAVRANLLRQGKYQHHDLKADATSENLTNLKRDEAF |
| | V372L- | VMNLITTCAFAANNIRNIIDPEQIKDILGKDDFIRSLKKDAFNKHIYGLFFP |
| | V501T- | VGAEMIEPVSLWAMRGAVQQIRNNVAHYKKDALATIFNITKFEYPGAEN |
| | G548A- | ILFSNTIYKQFFEKELEQLPEAFALQLKSGGVLAQYGFDQLKFLLDVVKF |
| | N722R- | HLCRTTVPFAPGFKKVMKGGTGYQNSKQDESFYNLELTAYQLKDDFSEE |
| | A737S- | AWSARYFLLKIIYNYLFLPEFTNNDKVFADTVAWLLGKNRQQAQSSKNK |
| | A762G- | NAYAFADIRLMESSESITDYMAYVQSQWIMEENKKEDDKKEDTRINFEK |
| | W765N- | FVLQLFVKGFDTFMKQSAYQFINTPQPQINPTDTNQQQADKLKALEKKIC |
| | S807R- | PFCHIQNNKIDPAKDEQIAFYVYCKLLDARHLSTLRNELIKYRASSESELFI |
| | S819K- | HLTEIIELCLLHADNVPGDYNQIYKNKEQCLQRLQPFIAENSDYTNWGDL |
| | N871K- | YVQTDDETPVVHRGIELSVKYGTAKLLEKLIASDAKFKITENEFDLWHN |
| | D974Y- | ARATIEAMIKKREEYHNLWLEAKEKDDKEKKTRIREKNNYARRFVETK |
| | I1064K | GEDYIDICDYIDRYNWLNNKLHFEHLKSLQNITIEMLGRMAGFVVLWER |
| | | DFQYLDQQRVKQQGGALMDFSHGVPKYEELKPHYAYFKRLFLCNNYSD |
| | | TRNHIAHFNYLTKSAANYSLIDLINQLRELLHYDRKLKNAVAKTFIDLFD |
| | | KHGMVLKLKFDSQMHQLQVGSITSKKIFHLGTKKESDNIYTFQVPTAYC |
| | | NMCKQLLEMKN |
| 259 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | M311V- | FLNHKFHLPVKYQKEGQWVQFELSTCILQAVEHQNLDELQPYYDWSKW |
| | K318Q- | YINDKSGKLKKSITNNHIQIGDGESKRQKALLMWETEFTDKGELDLETY |
| | S352C- | HEKFIIDTLAASLRSVEKGELRDNGTPKETNSYHRNLKKVLQNHQALIFG |
| | V372L- | RRETPNQENRDNKQLAIYNLEVVKYLEHYFPIKSNVRKNSAADIVYYLK |
| | V501T- | SETIKSTVTHQLENAVRANLLRQGKYQHHDLKADATSENLTNLKRDEAF |
| | G548A- | VMNLITTCAFAANNIRNIIDPEQIKDILGKDDFIRSLKKDAFNKHIYGLFFP |
| | I622N- | VGAEMIEPVSLWAMRGAVQQIRNNVAHYKKDALATIFNITKFEYPGAEN |
| | N722R- | ILFSNTIYKQFFEKELEQLPEAFALQLKSGGVLAQYGFDQLKFLLDVVKF |
| | A737S- | HLCRTTVPFAPGFKKVMKGGTGYQNSKQDESFYNLELTAYQLKDDFSEE |
| | A762G- | AWSARYFLLKIIYNYLFLPEFTNNDKVFADTVAWLLGKNRQQAQSSKNK |
| | W765N- | NAYAFADIRLMESSESITDYMAYVQSQWNMEENKKEDDKKEDTRINFE |
| | S807R- | KFVLQLFVKGFDTFMKQSAYQFINTPQPQINPTDTNQQQADKLKALEKKI |
| | S819K- | CPFCHIQNNKIDPAKDEQIAFYVYCKLLDARHLSTLRNELIKYRASSESEL |
| | N871K- | FIHLTEIIELCLLHADNVPGDYNQIYKNKEQCLQRLQPFIAENSDYTNWG |
| | D974Y- | DLYVQTDDETPVVHRGIELSVKYGTAKLLEKLIASDAKFKITENEFDLWH |
| | I1064K | NARATIEAMIKKREEYHNLWLEAKEKDDKEKKTRIREKNNYARRFVET |
| | | KGEDYIDICDYIDRYNWLNNKLHFEHLKSLQNITIEMLGRMAGFVVLWE |
| | | RDFQYLDQQRVKQQGGALMDFSHGVPKYEELKPHYAYFKRLFLCNNYS |
| | | DTRNHIAHFNYLTKSAANYSLIDLINQLRELLHYDRKLKNAVAKTFIDLF |
| | | DKHGMVLKLKFDSQMHQLQVGSITSKKIFHLGTKKESDNIYTFQVPTAY |
| | | CNMCKQLLEMKN |
| 260 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | M311V- | FLNHKFHLPVKYQKEGQWVQFELSTCILQAVEHQNLDELQPYYDWSKW |
| | K318Q- | YINDKSGKLKKSITNNHIQIGDGESKRQKALLMWETEFTDKGELDLETY |
| | S352C- | HEKFIIDTLAASLRSVEKGELRDNGTPKETNSYHRNLKKVLQNHQALIFG |
| | V372L- | RRETPNQENRDNKQLAIYNLEVVKYLEHYFPIKSNVRKNSAADIVYYLK |
| | V501T- | SETIKSTVTHQLENAVRANLLRQGKYQHHDLKADATSENLTNLKRDEAF |
| | G548A- | VMNLITTCAFAANNIRNIIDPEQIKDILGKDDFIRSLKKDAFNKHIYGLFFP |
| | N722R- | VGAEMIEPVSLWAMRGAVQQIRNNVAHYKKDALATIFNITKFEYPGAEN |
| | A737S- | ILFSNTIYKQFFEKELEQLPEAFALQLKSGGVLAQYGFDQLKFLLDVVKF |
| | W765N- | HLCRTTVPFAPGFKKVMKGGTGYQNSKQDESFYNLELTAYQLKDDFSEE |
| | D800R- | AWSARYFLLKIIYNYLFLPEFTNNDKVFADTVAWLLGKNRQQAQSSKNK |
| | S807R- | NAYAFADIRLMESSESITDYMAYVQSQWIMEENKKEDDKKEDTRINFEK |
| | S819K- | FVLQLFVKGFDTFMKQSAYQFINTPQPQINPTDTNQQQADKLKALEKKIC |
| | S827K- | PFCHIQNNKIDPAKDEQIAFYVYCKLLDARHLSTLRNELIKYRASSESELFI |
| | N871K- | HLTEIIELCLLHADNVPADYNQIYKNKEQCLQRLQPFIAENSDYTNWGDL |
| | D974Y- | YVQTDRETPVVHRGIELSVKYGTAKLLEKLIAKDAKFKITENEFDLWHN |
| | I1064K | ARATIEAMIKKREEYHNLWLEAKEKDDKEKKTRIREKNNYARRFVETK |
| | | GEDYIDICDYIDRYNWLNNKLHFEHLKSLQNITIEMLGRMAGFVVLWER |
| | | DFQYLDQQRVKQQGGALMDFSHGVPKYEELKPHYAYFKRLFLCNNYSD |
| | | TRNHIAHFNYLTKSAANYSLIDLINQLRELLHYDRKLKNAVAKTFIDLFD |
| | | KHGMVLKLKFDSQMHQLQVGSITSKKIFHLGTKKESDNIYTFQVPTAYC |
| | | NMCKQLLEMKN |
| 261 | H106Q- | MRVSKVKVTVNNKDTMVLMHRSAEKGALIYDTIDKKDRTEEIIPQKKQ |
| | H139Q- | KSFELSIQNKTLLRYEAIKKQKLDRSKYKVVEDIFKYRKLPAKITLEDILP |
| | D184E- | FLNHKFQLPVKYQKEGQWVQFELSTCILQAVEHQNLDELQPYYDWSKW |
| | A262T- | YINDKSGKLKKSITNNHIQIGDGESKRQKALLMWETEFTDKGELDLETY |
| | M311V- | HEKFIIDTLAASLRSVEKGELRDNGTPKETNSYHRNLKKVLQNHQALIFG |
| | K318Q- | RRETPNQENRDNKQLTIYNLEVVKYLEHYFPIKSNVRKNSAADIVYYLKS |
| | S352C- | ETIKSTVTHQLENAVRANLLRQGKYQHHDLKADATSENLTNLKRDEAF |
| | V372L- | VMNLITTCAFAANNIRNIIDPEQIKDILGKDDFIRSLKKDAFNKHIYGLFFP |
| | V501T- | VGAEMIEPVSLWAMRGAVQQIRNNVAHYKKDALATIFNITKFEYPGAEN |
| | G548A- | ILFSNTIYKQFFEKELEQLPEAFALQLKSGGVLAQYGFDQLKFLLDVVKF |
| | I622N- | HLCRTTVPFAPGFKKVMKGGTGYQNSKQDESFYNLELTAYQLKDDFSEE |
| | T726Q- | AWSARYFLLKIIYNYLFLPEFTNNDKVFADTVAWLLGKNRQQAQSSKNK |
| | N722R- | NAYAFADIRLMESSESITDYMAYVQSQWNMEENKKEDDKKEDTRINFE |
| | A737S- | KFVLQLFVKGFDTFMKQSAYQFINTPQPQINPTDTNQQQADKLKALEKKI |
| | A762G- | CPFCHIQNNKIDPAKDEQIAFYVYCKLLDARHLSQLRNELIKYRASSESEL |
| | W765N- | FIHLTEIIELCLLHADNVPGDYNQIYKNKEQCLQRLQPFIAENSDYTNWG |
| | S807R- | DLYVQTDDETPVVHRGIELSVKYGTAKLLEKLIAKDAKFKITENEFDLW |
| | S819K- | HNARATIEAMIKKREEYHNLWLEAKEKDDKEKKTRIREKNNYARRFVE |
| | S827K- | TKGEDYIDICDYIDRYNWLNNKLHFEHLKSLQNITIEMLGRMAGFVVLW |
| | N871K- | ERDFQYLDQQRVKQQGGALMDFSHGVPKYEELKPHYAYFKRLFLCNNY |
| | D974Y- | SDTRNHIAHFNYLTKSAANYSLIDLINQLRELLRYDRKLKNAVAKTFIDL |
| | H1020R- | FDKHGMVLKLKFDSQMHQLQVGSITSKKIFHLGTKKESDNIYTFQVPTA |
| | I1064K | YCNMCKQLLEMKN |
| 262 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | M311V- | FLNHKFHLPVKYQKEGQWVQFELSTCILQAVEHQNLDELQPYYDWSKW |
| | K318Q- | YINDKSGKLKKSITNNHIQIGDGESKRQKALLMWETEFTDKGELDLETY |
| | S352C- | HEKFIIDTLAASLRSVEKGELRDNGTPKETNSYHRNLKKVLQNHQALIFG |
| | V372L- | RRETPNQENRDNKQLAIYNLEVVKYLEHYFPIKSNVRKNSAADIVYYLK |
| | V501T- | SETIKSTVTHQLENAVRANLLRQGKYQHHDLKADATSENLTNLKRDEAF |
| | G548A- | VMNLITTCAFAANNIRNIIDPEQIKDILGKDDFIRSLKKDAFNKHIYGLFFP |
| | T726Q- | VGAEMIEPVSLWAMRGAVQQIRNNVAHYKKDALATIFNITKFEYPGAEN |
| | N722R- | ILFSNTIYKQFFEKELEQLPEAFALQLKSGGVLAQYGFDQLKFLLDVVKF |
| | A737S- | HLCRTTVPFAPGFKKVMKGGTGYQNSKQDESFYNLELTAYQLKDDFSEE |
| | A762G- | AWSARYFLLKIIYNYLFLPEFTNNDKVFADTVAWLLGKNRQQAQSSKNK |
| | W765N- | NAYAFADIRLMESSESITDYMAYVQSQWIMEENKKEDDKKEDTRINFEK |
| | S807R- | FVLQLFVKGFDTFMKQSAYQFINTPQPQINPTDTNQQQADKLKALEKKIC |
| | S819K- | PFCHIQNNKIDPAKDEQIAFYVYCKLLDARHLSQLRNELIKYRASSESELF |
| | N871K- | IHLTEIIELCLLHADNVPGDYNQIYKNKEQCLQRLQPFIAENSDYTNWGD |
| | D974Y- | LYVQTDDETPVVHRGIELSVKYGTAKLLEKLIASDAKFKITENEFDLWHN |
| | I1064K | ARATIEAMIKKREEYHNLWLEAKEKDDKEKKTRIREKNNYARRFVETK |
| | | GEDYIDICDYIDRYNWLNNKLHFEHLKSLQNITIEMLGRMAGFVVLWER |
| | | DFQYLDQQRVKQQGGALMDFSHGVPKYEELKPHYAYFKRLFLCNNYSD |
| | | TRNHIAHFNYLTKSAANYSLIDLINQLRELLHYDRKLKNAVAKTFIDLFD |
| | | KHGMVLKLKFDSQMHQLQVGSITSKKIFHLGTKKESDNIYTFQVPTAYC |
| | | NMCKQLLEMKN |
| 263 | H106Q- | MRVSKVKVTVNNKDTMVLMHRSAEKGALIYDTIDKKDRTEEIIPQKKQ |
| | H139Q- | KSFELSIQNKTLLRYEAIKKQKLDRSKYKVVEDIFKYRKLPAKITLEDILP |
| | D184E- | FLNHKFQLPVKYQKEGQWVQFELSTCILQAVEHQNLDELQPYYDWSKW |
| | M311V- | YINDKSGKLKKSITNNHIQIGDGESKRQKALLMWETEFTDKGELDLETY |
| | K318Q- | HEKFIIDTLAASLRSVEKGELRDNGTPKETNSYHRNLKKVLQNHQALIFG |
| | S352C- | RRETPNQENRDNKQLAIYNLEVVKYLEHYFPIKSNVRKNSAADIVYYLK |
| | V372L- | SETIKSTVTHQLENAVRANLLRQGKYQHHDLKADATSENLTNLKRDEAF |
| | V501T- | VMNLITTCAFAANNIRNIIDPEQIKDILGKDDFIRSLKKDAFNKHIYGLFFP |
| | G548A- | VGAEMIEPVSLWAMRGAVQQIRNNVAHYKKDALATIFNITKFEYPGAEN |
| | N722R- | ILFSNTIYKQFFEKELEQLPEAFALQLKSGGVLAQYGFDQLKFLLDVVKF |
| | A737S- | HLCRTTVPFAPGFKKVMKGGTGYQNSKQDESFYNLELTAYQLKDDFSEE |
| | A762G- | AWSARYFLLKIIYNYLFLPEFTNNDKVFADTVAWLLGKNRQQAQSSKNK |
| | W765N- | NAYAFADIRLMESSESITDYMAYVQSQWIMEENKKEDDKKEDTRINFEK |
| | S807R- | FVLQLFVKGFDTFMKQSAYQFINTPQPQINPTDTNQQQADKLKALEKKIC |
| | S819K- | PFCHIQNNKIDPAKDEQIAFYVYCKLLDARHLSTLRNELIKYRASSESELFI |
| | N871K- | HLTEIIELCLLHADNVPGDYNQIYKNKEQCLQRLQPFIAENSDYTNWGDL |
| | D974Y- | YVQTDDETPVVHRGIELSVKYGTAKLLEKLIASDAKFKITENEFDLWHN |
| | T1032V- | ARATIEAMIKKREEYHNLWLEAKEKDDKEKKTRIREKNNYARRFVETK |
| | I1064K | GEDYIDICDYIDRYNWLNNKLHFEHLKSLQNITIEMLGRMAGFVVLWER |
| | | DFQYLDQQRVKQQGGALMDFSHGVPKYEELKPHYAYFKRLFLCNNYSD |
| | | TRNHIAHFNYLTKSAANYSLIDLINQLRELLHYDRKLKNAVAKVFIDLFD |
| | | KHGMVLKLKFDSQMHQLQVGSITSKKIFHLGTKKESDNIYTFQVPTAYC |
| | | NMCKQLLEMKN |
| 264 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | M311V- | FLNHKFHLPVKYQKEGQWVQFELSTCILQAVEHQNLDELQPYYDWSKW |
| | K318Q- | YINDKSGKLKKSITNNHIQIGDGESKRQKALLMWETEFTDKGELDLETY |
| | S352C- | HEKFIIDTLAASLRSVEKGELRDNGTPKETNSYHRNLKKVLQNHQALIFG |
| | V372L- | RRETPNQENRDNKQLAIYNLEVVKYLEHYFPIKSNVRKNSAADIVYYLK |
| | V501T- | SETIKSTVTHQLENAVRANLLRQGKYQHHDLKADATSENLTNLKRDEAF |
| | G548A- | VMNLITTCAFAANNIRNIIDPEQIKDILGKDDFIRSLKKDAFNKHIYGLFFP |
| | N722R- | VGAEMIEPVSLWAMRGAVQQIRNNVAHYKKDALATIFNITKFEYPGAEN |
| | A737S- | ILFSNTIYKQFFEKELEQLPEAFALQLKSGGVLAQYGFDQLKFLLDVVKF |
| | W765N- | HLCRTTVPFAPGFKKVMKGGTGYQNSKQDESFYNLELTAYQLKDDFSEE |
| | S807R- | AWSARYFLLKIIYNYLFLPEFTNNDKVFADTVAWLLGKNRQQAQSSKNK |
| | S819K- | NAYAFADIRLMESSESITDYMAYVQSQWIMEENKKEDDKKEDTRINFEK |
| | N871K- | FVLQLFVKGFDTFMKQSAYQFINTPQPQINPTDTNQQQADKLKALEKKIC |
| | D974Y- | PFCHIQNNKIDPAKDEQIAFYVYCKLLDARHLSTLRNELIKYRASSESELFI |
| | H1020R- | HLTEIIELCLLHADNVPADYNQIYKNKEQCLQRLQPFIAENSDYTNWGDL |
| | I1064K | YVQTDDETPVVHRGIELSVKYGTAKLLEKLIASDAKFKITENEFDLWHN |
| | | ARATIEAMIKKREEYHNLWLEAKEKDDKEKKTRIREKNNYARRFVETK |
| | | GEDYIDICDYIDRYNWLNNKLHFEHLKSLQNITIEMLGRMAGFVVLWER |
| | | DFQYLDQQRVKQQGGALMDFSHGVPKYEELKPHYAYFKRLFLCNNYSD |
| | | TRNHIAHFNYLTKSAANYSLIDLINQLRELLRYDRKLKNAVAKTFIDLFD |
| | | KHGMVLKLKFDSQMHQLQVGSITSKKIFHLGTKKESDNIYTFQVPTAYC |
| | | NMCKQLLEMKN |
| 265 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | M311V- | FLNHKFHLPVKYQKEGQWVQFELSTCILQAVEHQNLDELQPYYDWSKW |
| | K318Q- | YINDKSGKLKKSITNNHIQIGDGESKRQKALLMWETEFTDKGELDLETY |
| | S352C- | HEKFIIDTLAASLRSVEKGELRDNGTPKETNSYHRNLKKVLQNHQALIFG |
| | V372L- | RRETPNQENRDNKQLAIYNLEVVKYLEHYFPIKSNVRKNSAADIVYYLK |
| | V501T- | SETIKSTVTHQLENAVRANLLRQGKYQHHDLKADATSENLTNLKRDEAF |
| | G548A- | VMNLITTCAFAANNIRNIIDPEQIKDILGKDDFIRSLKKDAFNKHIYGLFFP |
| | N722R- | VGAEMIEPVSLWAMRGAVQQIRNNVAHYKKDALATIFNITKFEYPGAEN |
| | A737S- | ILFSNTIYKQFFEKELEQLPEAFALQLKSGGVLAQYGFDQLKFLLDVVKF |
| | W765N- | HLCRTTVPFAPGFKKVMKGGTGYQNSKQDESFYNLELTAYQLKDDFSEE |
| | S807R- | AWSARYFLLKIIYNYLFLPEFTNNDKVFADTVAWLLGKNRQQAQSSKNK |
| | S819K- | NAYAFADIRLMESSESITDYMAYVQSQWIMEENKKEDDKKEDTRINFEK |
| | S827K- | FVLQLFVKGFDTFMKQSAYQFINTPQPQINPTDTNQQQADKLKALEKKIC |
| | N871K- | PFCHIQNNKIDPAKDEQIAFYVYCKLLDARHLSTLRNELIKYRASSESELFI |
| | D974Y- | HLTEIIELCLLHADNVPADYNQIYKNKEQCLQRLQPFIAENSDYTNWGDL |
| | I1064K | YVQTDDETPVVHRGIELSVKYGTAKLLEKLIAKDAKFKITENEFDLWHN |
| | | ARATIEAMIKKREEYHNLWLEAKEKDDKEKKTRIREKNNYARRFVETK |
| | | GEDYIDICDYIDRYNWLNNKLHFEHLKSLQNITIEMLGRMAGFVVLWER |
| | | DFQYLDQQRVKQQGGALMDFSHGVPKYEELKPHYAYFKRLFLCNNYSD |
| | | TRNHIAHFNYLTKSAANYSLIDLINQLRELLHYDRKLKNAVAKTFIDLFD |
| | | KHGMVLKLKFDSQMHQLQVGSITSKKIFHLGTKKESDNIYTFQVPTAYC |
| | | NMCKQLLEMKN |
| 266 | H106Q- | MRVSKVKVTVNNKDTMVLMHRSAEKGALIYDTIDKKDRTEEIIPQKKQ |
| | H139Q- | KSFELSIQNKTLLRYEAIKKQKLDRSKYKVVEDIFKYRKLPAKITLEDILP |
| | G154K- | FLNHKFQLPVKYQKEGQWVQFELSTCILQAVEHQNLDELQPYYDWSKW |
| | D184E- | YINDKSKKLKKSITNNHIQIGDGESKRQKALLMWETEFTDKGELDLETY |
| | M311V- | HEKFIIDTLAASLRSVEKGELRDNGTPKETNSYHRNLKKVLQNHQALIFG |
| | K318Q- | RRETPNQENRDNKQLAIYNLEVVKYLEHYFPIKSNVRKNSAADIVYYLK |
| | S352C- | SETIKSTVTHQLENAVRANLLRQGKYQHHDLKADATSENLTNLKRDEAF |
| | V372L- | VMNLITTCAFAANNIRNIIDPEQIKDILGKDDFIRSLKKDAFNKHIYGLFFP |
| | V501T- | VGAEMIEPVSLWAMRGAVQQIRNNVAHYKKDALATIFNITKFEYPGAEN |
| | G548A- | ILFSNTIYKQFFEKELEQLPEAFALQLKSGGVLAQYGFDQLKFLLDVVKF |
| | T726Q- | HLCRTTVPFAPGFKKVMKGGTGYQNSKQDESFYNLELTAYQLKDDFSEE |
| | N722R- | AWSARYFLLKIIYNYLFLPEFTNNDKVFADTVAWLLGKNRQQAQSSKNK |
| | A737S- | NAYAFADIRLMESSESITDYMAYVQSQWIMEENKKEDDKKEDTRINFEK |
| | W765N- | FVLQLFVKGFDTFMKQSAYQFINTPQPQINPTDTNQQQADKLKALEKKIC |
| | D800R- | PFCHIQNNKIDPAKDEQIAFYVYCKLLDARHLSQLRNELIKYRASSESELF |
| | S807R- | IHLTEIIELCLLHADNVPADYNQIYKNKEQCLQRLQPFIAENSDYTNWGD |
| | S819K- | LYVQTDRETPVVHRGIELSVKYGTAKLLEKLIASDAKFKITENEFDLWHN |
| | N871K- | ARATIEAMIKKREEYHNLWLEAKEKDDKEKKTRIREKNNYARRFVETK |
| | D974Y- | GEDYIDICDYIDRYNWLNNKLHFEHLKSLQNITIEMLGRMAGFVVLWER |
| | I1064K | DFQYLDQQRVKQQGGALMDFSHGVPKYEELKPHYAYFKRLFLCNNYSD |
| | | TRNHIAHFNYLTKSAANYSLIDLINQLRELLHYDRKLKNAVAKTFIDLFD |
| | | KHGMVLKLKFDSQMHQLQVGSITSKKIFHLGTKKESDNIYTFQVPTAYC |
| | | NMCKQLLEMKN |
| 267 | H139Q- | MRVSKVKVTVNNKDTMVLMHRSAEKGALIYDTIDKKDRTEEIIPQKKQ |
| | G154K- | KSFELSIQNKTLLRYEAIKKQKLDRSKYKVVEDIFKYRKLPAKITLEDILP |
| | D184E- | FLNHKFHLPVKYQKEGQWVQFELSTCILQAVEHQNLDELQPYYDWSKW |
| | A262T- | YINDKSKKLKKSITNNHIQIGDGESKRQKALLMWETEFTDKGELDLETY |
| | M311V- | HEKFIIDTLAASLRSVEKGELRDNGTPKETNSYHRNLKKVLQNHQALIFG |
| | K318Q- | RRETPNQENRDNKQLTIYNLEVVKYLEHYFPIKSNVRKNSAADIVYYLKS |
| | S352C- | ETIKSTVTHQLENAVRANLLRQGKYQHHDLKADATSENLTNLKRDEAF |
| | V372L- | VMNLITTCAFAANNIRNIIDPEQIKDILGKDDFIRSLKKDAFNKHIYGLFFP |
| | V501T- | VGAEMIEPVSLWAMRGAVQQIRNNVAHYKKDALATIFNITKFEYPGAEN |
| | G548A- | ILFSNTIYKQFFEKELEQLPEAFALQLKSGGVLAQYGFDQLKFLLDVVKF |
| | T726Q- | HLCRTTVPFAPGFKKVMKGGTGYQNSKQDESFYNLELTAYQLKDDFSEE |
| | N722R- | AWSARYFLLKIIYNYLFLPEFTNNDKVFADTVAWLLGKNRQQAQSSKNK |
| | A737S- | NAYAFADIRLMESSESITDYMAYVQSQWIMEENKKEDDKKEDTRINFEK |
| | W765N- | FVLQLFVKGFDTFMKQSAYQFINTPQPQINPTDTNQQQADKLKALEKKIC |
| | S807R- | PFCHIQNNKIDPAKDEQIAFYVYCKLLDARHLSQLRNELIKYRASSESELF |
| | S819K- | IHLTEIIELCLLHADNVPADYNQIYKNKEQCLQRLQPFIAENSDYTNWGD |
| | N871K- | LYVQTDDETPVVHRGIELSVKYGTAKLLEKLIASDAKFKITENEFDLWHN |
| | D974Y- | ARATIEAMIKKREEYHNLWLEAKEKDDKEKKTRIREKNNYARRFVETK |
| | T1032V- | GEDYIDICDYIDRYNWLNNKLHFEHLKSLQNITIEMLGRMAGFVVLWER |
| | I1064K | DFQYLDQQRVKQQGGALMDFSHGVPKYEELKPHYAYFKRLFLCNNYSD |
| | | TRNHIAHFNYLTKSAANYSLIDLINQLRELLHYDRKLKNAVAKVFIDLFD |
| | | KHGMVLKLKFDSQMHQLQVGSITSKKIFHLGTKKESDNIYTFQVPTAYC |
| | | NMCKQLLEMKN |
| 268 | H139Q- | MRVSKVKVTVNNKDTMVLMHRSAEKGALIYDTIDKKDRTEEIIPQKKQ |
| | G154K- | KSFELSIQNKTLLRYEAIKKQKLDRSKYKVVEDIFKYRKLPAKITLEDILP |
| | D184E- | FLNHKFHLPVKYQKEGQWVQFELSTCILQAVEHQNLDELQPYYDWSKW |
| | M311V- | YINDKSKKLKKSITNNHIQIGDGESKRQKALLMWETEFTDKGELDLETY |
| | K318Q- | HEKFIIDTLAASLRSVEKGELRDNGTPKETNSYHRNLKKVLQNHQALIFG |
| | S352C- | RRETPNQENRDNKQLAIYNLEVVKYLEHYFPIKSNVRKNSAADIVYYLK |
| | V372L- | SETIKSTVTHQLENAVRANLLRQGKYQHHDLKADATSENLTNLKRDEAF |
| | V501T- | VMNLITTCAFAANNIRNIIDPEQIKDILGKDDFIRSLKKDAFNKHIYGLFFP |
| | G548A- | VGAEMIEPVSLWAMRGAVQQIRNNVAHYKKDALATIFNITKFEYPGAEN |
| | N722R- | ILFSNTIYKQFFEKELEQLPEAFALQLKSGGVLAQYGFDQLKFLLDVVKF |
| | A737S- | HLCRTTVPFAPGFKKVMKGGTGYQNSKQDESFYNLELTAYQLKDDFSEE |
| | W765N- | AWSARYFLLKIIYNYLFLPEFTNNDKVFADTVAWLLGKNRQQAQSSKNK |
| | S807R- | NAYAFADIRLMESSESITDYMAYVQSQWIMEENKKEDDKKEDTRINFEK |
| | S819K- | FVLQLFVKGFDTFMKQSAYQFINTPQPQINPTDTNQQQADKLKALEKKIC |
| | N871K- | PFCHIQNNKIDPAKDEQIAFYVYCKLLDARHLSTLRNELIKYRASSESELFI |
| | D974Y- | HLTEIIELCLLHADNVPADYNQIYKNKEQCLQRLQPFIAENSDYTNWGDL |
| | I1064K | YVQTDDETPVVHRGIELSVKYGTAKLLEKLIASDAKFKITENEFDLWHN |
| | | ARATIEAMIKKREEYHNLWLEAKEKDDKEKKTRIREKNNYARRFVETK |
| | | GEDYIDICDYIDRYNWLNNKLHFEHLKSLQNITIEMLGRMAGFVVLWER |
| | | DFQYLDQQRVKQQGGALMDFSHGVPKYEELKPHYAYFKRLFLCNNYSD |
| | | TRNHIAHFNYLTKSAANYSLIDLINQLRELLHYDRKLKNAVAKTFIDLFD |
| | | KHGMVLKLKFDSQMHQLQVGSITSKKIFHLGTKKESDNIYTFQVPTAYC |
| | | NMCKQLLEMKN |
| 269 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | A262T- | FLNHKFHLPVKYQKEGQWVQFELSTCILQAVEHQNLDELQPYYDWSKW |
| | M311V- | YINDKSGKLKKSITNNHIQIGDGESKRQKALLMWETEFTDKGELDLETY |
| | K318Q- | HEKFIIDTLAASLRSVEKGELRDNGTPKETNSYHRNLKKVLQNHQALIFG |
| | S352C- | RRETPNQENRDNKQLTIYNLEVVKYLEHYFPIKSNVRKNSAADIVYYLKS |
| | V372L- | ETIKSTVTHQLENAVRANLLRQGKYQHHDLKADATSENLTNLKRDEAF |
| | V501T- | VMNLITTCAFAANNIRNIIDPEQIKDILGKDDFIRSLKKDAFNKHIYGLFFP |
| | G548A- | VGAEMIEPVSLWAMRGAVQQIRNNVAHYKKDALATIFNITKFEYPGAEN |
| | W621Q- | ILFSNTIYKQFFEKELEQLPEAFALQLKSGGVLAQYGFDQLKFLLDVVKF |
| | N722R- | HLCRTTVPFAPGFKKVMKGGTGYQNSKQDESFYNLELTAYQLKDDFSEE |
| | A737S- | AWSARYFLLKIIYNYLFLPEFTNNDKVFADTVAWLLGKNRQQAQSSKNK |
| | W765N- | NAYAFADIRLMESSESITDYMAYVQSQQIMEENKKEDDKKEDTRINFEK |
| | D800R- | FVLQLFVKGFDTFMKQSAYQFINTPQPQINPTDTNQQQADKLKALEKKIC |
| | S807R- | PFCHIQNNKIDPAKDEQIAFYVYCKLLDARHLSTLRNELIKYRASSESELFI |
| | S819K- | HLTEIIELCLLHADNVPADYNQIYKNKEQCLQRLQPFIAENSDYTNWGDL |
| | S827K- | YVQTDRETPVVHRGIELSVKYGTAKLLEKLIAKDAKFKITENEFDLWHN |
| | N871K- | ARATIEAMIKKREEYHNLWLEAKEKDDKEKKTRIREKNNYARRFVETK |
| | D974Y- | GEDYIDICDYIDRYNWLNNKLHFEHLKSLQNITIEMLGRMAGFVVLWER |
| | I1064K | DFQYLDQQRVKQQGGALMDFSHGVPKYEELKPHYAYFKRLFLCNNYSD |
| | | TRNHIAHFNYLTKSAANYSLIDLINQLRELLHYDRKLKNAVAKTFIDLFD |
| | | KHGMVLKLKFDSQMHQLQVGSITSKKIFHLGTKKESDNIYTFQVPTAYC |
| | | NMCKQLLEMKN |
| 270 | H106Q- | MRVSKVKVTVNNKDTMVLMHRSAEKGALIYDTIDKKDRTEEIIPQKKQ |
| | H139Q- | KSFELSIQNKTLLRYEAIKKQKLDRSKYKVVEDIFKYRKLPAKITLEDILP |
| | D184E- | FLNHKFQLPVKYQKEGQWVQFELSTCILQAVEHQNLDELQPYYDWSKW |
| | M311V- | YINDKSGKLKKSITNNHIQIGDGESKRQKALLMWETEFTDKGELDLETY |
| | K318Q- | HEKFIIDTLAASLRSVEKGELRDNGTPKETNSYHRNLKKVLQNHQALIFG |
| | S352C- | RRETPNQENRDNKQLAIYNLEVVKYLEHYFPIKSNVRKNSAADIVYYLK |
| | V372L- | SETIKSTVTHQLENAVRANLLRQGKYQHHDLKADATSENLTNLKRDEAF |
| | V501T- | VMNLITTCAFAANNIRNIIDPEQIKDILGKDDFIRSLKKDAFNKHIYGLFFP |
| | G548A- | VGAEMIEPVSLWAMRGAVQQIRNNVAHYKKDALATIFNITKFEYPGAEN |
| | N722R- | ILFSNTIYKQFFEKELEQLPEAFALQLKSGGVLAQYGFDQLKFLLDVVKF |
| | A737S- | HLCRTTVPFAPGFKKVMKGGTGYQNSKQDESFYNLELTAYQLKDDFSEE |
| | W765N- | AWSARYFLLKIIYNYLFLPEFTNNDKVFADTVAWLLGKNRQQAQSSKNK |
| | S807R- | NAYAFADIRLMESSESITDYMAYVQSQWIMEENKKEDDKKEDTRINFEK |
| | S819K- | FVLQLFVKGFDTFMKQSAYQFINTPQPQINPTDTNQQQADKLKALEKKIC |
| | N871K- | PFCHIQNNKIDPAKDEQIAFYVYCKLLDARHLSTLRNELIKYRASSESELFI |
| | D974Y- | HLTEIIELCLLHADNVPADYNQIYKNKEQCLQRLQPFIAENSDYTNWGDL |
| | H1020R- | YVQTDDETPVVHRGIELSVKYGTAKLLEKLIASDAKFKITENEFDLWHN |
| | I1064K | ARATIEAMIKKREEYHNLWLEAKEKDDKEKKTRIREKNNYARRFVETK |
| | | GEDYIDICDYIDRYNWLNNKLHFEHLKSLQNITIEMLGRMAGFVVLWER |
| | | DFQYLDQQRVKQQGGALMDFSHGVPKYEELKPHYAYFKRLFLCNNYSD |
| | | TRNHIAHFNYLTKSAANYSLIDLINQLRELLRYDRKLKNAVAKTFIDLFD |
| | | KHGMVLKLKFDSQMHQLQVGSITSKKIFHLGTKKESDNIYTFQVPTAYC |
| | | NMCKQLLEMKN |
| 271 | H139Q- | MRVSKVKVTVNNKDTMVLMHRSAEKGALIYDTIDKKDRTEEIIPQKKQ |
| | G154K- | KSFELSIQNKTLLRYEAIKKQKLDRSKYKVVEDIFKYRKLPAKITLEDILP |
| | D184E- | FLNHKFHLPVKYQKEGQWVQFELSTCILQAVEHQNLDELQPYYDWSKW |
| | M311V- | YINDKSKKLKKSITNNHIQIGDGESKRQKALLMWETEFTDKGELDLETY |
| | K318Q- | HEKFIIDTLAASLRSVEKGELRDNGTPKETNSYHRNLKKVLQNHQALIFG |
| | S352C- | RRETPNQENRDNKQLAIYNLEVVKYLEHYFPIKSNVRKNSAADIVYYLK |
| | V372L- | SETIKSTVTHQLENAVRANLLRQGKYQHHDLKADATSENLTNLKRDEAF |
| | V501T- | VMNLITTCAFAANNIRNIIDPEQIKDILGKDDFIRSLKKDAFNKHIYGLFFP |
| | G548A- | VGAEMIEPVSLWAMRGAVQQIRNNVAHYKKDALATIFNITKFEYPGAEN |
| | N722R- | ILFSNTIYKQFFEKELEQLPEAFALQLKSGGVLAQYGFDQLKFLLDVVKF |
| | A737S- | HLCRTTVPFAPGFKKVMKGGTGYQNSKQDESFYNLELTAYQLKDDFSEE |
| | W765N- | AWSARYFLLKIIYNYLFLPEFTNNDKVFADTVAWLLGKNRQQAQSSKNK |
| | D800R- | NAYAFADIRLMESSESITDYMAYVQSQWIMEENKKEDDKKEDTRINFEK |
| | S807R- | FVLQLFVKGFDTFMKQSAYQFINTPQPQINPTDTNQQQADKLKALEKKIC |
| | S819K- | PFCHIQNNKIDPAKDEQIAFYVYCKLLDARHLSTLRNELIKYRASSESELFI |
| | S827K- | HLTEIIELCLLHADNVPADYNQIYKNKEQCLQRLQPFIAENSDYTNWGDL |
| | N871K- | YVQTDRETPVVHRGIELSVKYGTAKLLEKLIAKDAKFKITENEFDLWHN |
| | D974Y- | ARATIEAMIKKREEYHNLWLEAKEKDDKEKKTRIREKNNYARRFVETK |
| | T1032V- | GEDYIDICDYIDRYNWLNNKLHFEHLKSLQNITIEMLGRMAGFVVLWER |
| | I1064K | DFQYLDQQRVKQQGGALMDFSHGVPKYEELKPHYAYFKRLFLCNNYSD |
| | | TRNHIAHFNYLTKSAANYSLIDLINQLRELLHYDRKLKNAVAKVFIDLFD |
| | | KHGMVLKLKFDSQMHQLQVGSITSKKIFHLGTKKESDNIYTFQVPTAYC |
| | | NMCKQLLEMKN |
| 272 | H106Q- | MRVSKVKVTVNNKDTMVLMHRSAEKGALIYDTIDKKDRTEEIIPQKKQ |
| | H139Q- | KSFELSIQNKTLLRYEAIKKQKLDRSKYKVVEDIFKYRKLPAKITLEDILP |
| | G154K- | FLNHKFQLPVKYQKEGQWVQFELSTCILQAVEHQNLDELQPYYDWSKW |
| | D184E- | YINDKSKKLKKSITNNHIQIGDGESKRQKALLMWETEFTDKGELDLETY |
| | M311V- | HEKFIIDTLAASLRSVEKGELRDNGTPKETNSYHRNLKKVLQNHQALIFG |
| | K318Q- | RRETPNQENRDNKQLAIYNLEVVKYLEHYFPIKSNVRKNSAADIVYYLK |
| | S352C- | SETIKSTVTHQLENAVRANLLRQGKYQHHDLKADATSENLTNLKRDEAF |
| | V372L- | VMNLITTCAFAANNIRNIIDPEQIKDILGKDDFIRSLKKDAFNKHIYGLFFP |
| | V501T- | VGAEMIEPVSLWAMRGAVQQIRNNVAHYKKDALATIFNITKFEYPGAEN |
| | G548A- | ILFSNTIYKQFFEKELEQLPEAFALQLKSGGVLAQYGFDQLKFLLDVVKF |
| | W621Q- | HLCRTTVPFAPGFKKVMKGGTGYQNSKQDESFYNLELTAYQLKDDFSEE |
| | N722R- | AWSARYFLLKIIYNYLFLPEFTNNDKVFADTVAWLLGKNRQQAQSSKNK |
| | A737S- | NAYAFADIRLMESSESITDYMAYVQSQQIMEENKKEDDKKEDTRINFEK |
| | A762G- | FVLQLFVKGFDTFMKQSAYQFINTPQPQINPTDTNQQQADKLKALEKKIC |
| | W765N- | PFCHIQNNKIDPAKDEQIAFYVYCKLLDARHLSTLRNELIKYRASSESELFI |
| | S807R- | HLTEIIELCLLHADNVPGDYNQIYKNKEQCLQRLQPFIAENSDYTNWGDL |
| | S819K- | YVQTDDETPVVHRGIELSVKYGTAKLLEKLIAKDAKFKITENEFDLWHN |
| | S827K- | ARATIEAMIKKREEYHNLWLEAKEKDDKEKKTRIREKNNYARRFVETK |
| | N871K- | GEDYIDICDYIDRYNWLNNKLHFEHLKSLQNITIEMLGRMAGFVVLWER |
| | D974Y- | DFQYLDQQRVKQQGGALMDFSHGIPKYEELKPHYAYFKRLFLCNNYSD |
| | I1064K | TRNHIAHFNYLTKSAANYSLIDLINQLRELLHYDRKLKNAVAKTFIDLFD |
| | | KHGMVLKLKFDSQMHQLQVGSITSKKIFHLGTKKESDNIYTFQVPTAYC |
| | | NMCKQLLEMKN |
| 273 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | M311V- | FLNHKFHLPVKYQKEGQWVQFELSTCILQAVEHQNLDELQPYYDWSKW |
| | K318Q- | YINDKSGKLKKSITNNHIQIGDGESKRQKALLMWETEFTDKGELDLETY |
| | S352C- | HEKFIIDTLAASLRSVEKGELRDNGTPKETNSYHRNLKKVLQNHQALIFG |
| | V372L- | RRETPNQENRDNKQLAIYNLEVVKYLEHYFPIKSNVRKNSAADIVYYLK |
| | V501T- | SETIKSTVTHQLENAVRANLLRQGKYQHHDLKADATSENLTNLKRDEAF |
| | G548A- | VMNLITTCAFAANNIRNIIDPEQIKDILGKDDFIRSLKKDAFNKHIYGLFFP |
| | N722R- | VGAEMIEPVSLWAMRGAVQQIRNNVAHYKKDALATIFNITKFEYPGAEN |
| | A737S- | ILFSNTIYKQFFEKELEQLPEAFALQLKSGGVLAQYGFDQLKFLLDVVKF |
| | A762G- | HLCRTTVPFAPGFKKVMKGGTGYQNSKQDESFYNLELTAYQLKDDFSEE |
| | W765N- | AWSARYFLLKIIYNYLFLPEFTNNDKVFADTVAWLLGKNRQQAQSSKNK |
| | S807R- | NAYAFADIRLMESSESITDYMAYVQSQWIMEENKKEDDKKEDTRINFEK |
| | S819K- | FVLQLFVKGFDTFMKQSAYQFINTPQPQINPTDTNQQQADKLKALEKKIC |
| | S827K- | PFCHIQNNKIDPAKDEQIAFYVYCKLLDARHLSTLRNELIKYRASSESELFI |
| | N871K- | HLTEIIELCLLHADNVPGDYNQIYKNKEQCLQRLQPFIAENSDYTNWGDL |
| | D974Y- | YVQTDDETPVVHRGIELSVKYGTAKLLEKLIAKDAKFKITENEFDLWHN |
| | I1064K | ARATIEAMIKKREEYHNLWLEAKEKDDKEKKTRIREKNNYARRFVETK |
| | | GEDYIDICDYIDRYNWLNNKLHFEHLKSLQNITIEMLGRMAGFVVLWER |
| | | DFQYLDQQRVKQQGGALMDFSHGVPKYEELKPHYAYFKRLFLCNNYSD |
| | | TRNHIAHFNYLTKSAANYSLIDLINQLRELLHYDRKLKNAVAKTFIDLFD |
| | | KHGMVLKLKFDSQMHQLQVGSITSKKIFHLGTKKESDNIYTFQVPTAYC |
| | | NMCKQLLEMKN |
| 274 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | M311V- | FLNHKFHLPVKYQKEGQWVQFELSTCILQAVEHQNLDELQPYYDWSKW |
| | K318Q- | YINDKSGKLKKSITNNHIQIGDGESKRQKALLMWETEFTDKGELDLETY |
| | S352C- | HEKFIIDTLAASLRSVEKGELRDNGTPKETNSYHRNLKKVLQNHQALIFG |
| | V372L- | RRETPNQENRDNKQLAIYNLEVVKYLEHYFPIKSNVRKNSAADIVYYLK |
| | V501T- | SETIKSTVTHQLENAVRANLLRQGKYQHHDLKADATSENLTNLKRDEAF |
| | E543L- | VMNLITTCAFAANNIRNIIDPEQIKDILGKDDFIRSLKKDAFNKHIYGLFFP |
| | G548A- | VGAEMIEPVSLWAMRGAVQQIRNNVAHYKKDALATIFNITKFEYPGAEN |
| | I622N- | ILFSNTIYKQFFEKELEQLPEAFALQLKSGGVLAQYGFDQLKFLLDVVKF |
| | N722R- | HLCRTTVPFAPGFKKVMKGGTGYQNSKQDESFYNLELTAYQLKDDFSLE |
| | A737S- | AWSARYFLLKIIYNYLFLPEFTNNDKVFADTVAWLLGKNRQQAQSSKNK |
| | W765N- | NAYAFADIRLMESSESITDYMAYVQSQWNMEENKKEDDKKEDTRINFE |
| | D800R- | KFVLQLFVKGFDTFMKQSAYQFINTPQPQINPTDTNQQQADKLKALEKKI |
| | S807R- | CPFCHIQNNKIDPAKDEQIAFYVYCKLLDARHLSTLRNELIKYRASSESEL |
| | S819K- | FIHLTEIIELCLLHADNVPADYNQIYKNKEQCLQRLQPFIAENSDYTNWG |
| | S827K- | DLYVQTDRETPVVHRGIELSVKYGTAKLLEKLIAKDAKFKITENEFDLW |
| | N871K- | HNARATIEAMIKKREEYHNLWLEAKEKDDKEKKTRIREKNNYARRFVE |
| | S919K- | TKGEDYIDICDYIDRYNWLNNKLHFEHLKKLQNITIEMLGRMAGFVVLW |
| | D974Y- | ERDFQYLDQQRVKQQGGALMDFSHGVPKYEELKPHYAYFKRLFLCNNY |
| | I1064K | SDTRNHIAHFNYLTKSAANYSLIDLINQLRELLHYDRKLKNAVAKTFIDL |
| | | FDKHGMVLKLKFDSQMHQLQVGSITSKKIFHLGTKKESDNIYTFQVPTA |
| | | YCNMCKQLLEMKN |
| 275 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | M311V- | FLNHKFHLPVKYQKEGQWVQFELSTCILQAVEHQNLDELQPYYDWSKW |
| | K318Q- | YINDKSGKLKKSITNNHIQIGDGESKRQKALLMWETEFTDKGELDLETY |
| | S352C- | HEKFIIDTLAASLRSVEKGELRDNGTPKETNSYHRNLKKVLQNHQALIFG |
| | V372L- | RRETPNQENRDNKQLAIYNLEVVKYLEHYFPIKSNVRKNSAADIVYYLK |
| | V501T- | SETIKSTVTHQLENAVRANLLRQGKYQHHDLKADATSENLTNLKRDEAF |
| | G548A- | VMNLITTCAFAANNIRNIIDPEQIKDILGKDDFIRSLKKDAFNKHIYGLFFP |
| | I622N- | VGAEMIEPVSLWAMRGAVQQIRNNVAHYKKDALATIFNITKFEYPGAEN |
| | N722R- | ILFSNTIYKQFFEKELEQLPEAFALQLKSGGVLAQYGFDQLKFLLDVVKF |
| | A737S- | HLCRTTVPFAPGFKKVMKGGTGYQNSKQDESFYNLELTAYQLKDDFSEE |
| | W765N- | AWSARYFLLKIIYNYLFLPEFINNDKVFADTVAWLLGKNRQQAQSSKNK |
| | S807R- | NAYAFADIRLMESSESITDYMAYVQSQWNMEENKKEDDKKEDTRINFE |
| | S819K- | KFVLQLFVKGFDTFMKQSAYQFINTPQPQINPTDTNQQQADKLKALEKKI |
| | N871K- | CPFCHIQNNKIDPAKDEQIAFYVYCKLLDARHLSTLRNELIKYRASSESEL |
| | D974Y- | FIHLTEIIELCLLHADNVPADYNQIYKNKEQCLQRLQPFIAENSDYTNWG |
| | I1064K | DLYVQTDDETPVVHRGIELSVKYGTAKLLEKLIASDAKFKITENEFDLWH |
| | | NARATIEAMIKKREEYHNLWLEAKEKDDKEKKTRIREKNNYARRFVET |
| | | KGEDYIDICDYIDRYNWLNNKLHFEHLKSLQNITIEMLGRMAGFVVLWE |
| | | RDFQYLDQQRVKQQGGALMDFSHGVPKYEELKPHYAYFKRLFLCNNYS |
| | | DTRNHIAHFNYLTKSAANYSLIDLINQLRELLHYDRKLKNAVAKTFIDLF |
| | | DKHGMVLKLKFDSQMHQLQVGSITSKKIFHLGTKKESDNIYTFQVPTAY |
| | | CNMCKQLLEMKN |
| 276 | H139Q- | MRVSKVKVTVNNKDTMVLMHRSAEKGALIYDTIDKKDRTEEIIPQKKQ |
| | G154K- | KSFELSIQNKTLLRYEAIKKQKLDRSKYKVVEDIFKYRKLPAKITLEDILP |
| | D184E- | FLNHKFHLPVKYQKEGQWVQFELSTCILQAVEHQNLDELQPYYDWSKW |
| | M311V- | YINDKSKKLKKSITNNHIQIGDGESKRQKALLMWETEFTDKGELDLETY |
| | K318Q- | HEKFIIDTLAASLRSVEKGELRDNGTPKETNSYHRNLKKVLQNHQALIFG |
| | S352C- | RRETPNQENRDNKQLAIYNLEVVKYLEHYFPIKSNVRKNSAADIVYYLK |
| | V372L- | SETIKSTVTHQLENAVRANLLRQGKYQHHDLKADATSENLTNLKRDEAF |
| | V501T- | VMNLITTCAFAANNIRNIIDPEQIKDILGKDDFIRSLKKDAFNKHIYGLFFP |
| | G548A- | VGAEMIEPVSLWAMRGAVQQIRNNVAHYKKDALATIFNITKFEYPGAEN |
| | N722R- | ILFSNTIYKQFFEKELEQLPEAFALQLKSGGVLAQYGFDQLKFLLDVVKF |
| | A737S- | HLCRTTVPFAPGFKKVMKGGTGYQNSKQDESFYNLELTAYQLKDDFSEE |
| | W765N- | AWSARYFLLKIIYNYLFLPEFTNNDKVFADTVAWLLGKNRQQAQSSKNK |
| | D800R- | NAYAFADIRLMESSESITDYMAYVQSQWIMEENKKEDDKKEDTRINFEK |
| | S807R- | FVLQLFVKGFDTFMKQSAYQFINTPQPQINPTDTNQQQADKLKALEKKIC |
| | S819K- | PFCHIQNNKIDPAKDEQIAFYVYCKLLDARHLSTLRNELIKYRASSESELFI |
| | S827K- | HLTEIIELCLLHADNVPADYNQIYKNKEQCLQRLQPFIAENSDYTNWGDL |
| | N871K- | YVQTDRETPVVHRGIELSVKYGTAKLLEKLIAKDAKFKITENEFDLWHN |
| | D974Y- | ARATIEAMIKKREEYHNLWLEAKEKDDKEKKTRIREKNNYARRFVETK |
| | T1032V- | GEDYIDICDYIDRYNWLNNKLHFEHLKSLQNITIEMLGRMAGFVVLWER |
| | I1064K | DFQYLDQQRVKQQGGALMDFSHGVPKYEELKPHYAYFKRLFLCNNYSD |
| | | TRNHIAHFNYLTKSAANYSLIDLINQLRELLHYDRKLKNAVAKVFIDLFD |
| | | KHGMVLKLKFDSQMHQLQVGSITSKKIFHLGTKKESDNIYTFQVPTAYC |
| | | NMCKQLLEMKN |
| 277 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | M311V- | FLNHKFHLPVKYQKEGQWVQFELSTCILQAVEHQNLDELQPYYDWSKW |
| | K318Q- | YINDKSGKLKKSITNNHIQIGDGESKRQKALLMWETEFTDKGELDLETY |
| | S352C- | HEKFIIDTLAASLRSVEKGELRDNGTPKETNSYHRNLKKVLQNHQALIFG |
| | V372L- | RRETPNQENRDNKQLAIYNLEVVKYLEHYFPIKSNVRKNSAADIVYYLK |
| | V501T- | SETIKSTVTHQLENAVRANLLRQGKYQHHDLKADATSENLTNLKRDEAF |
| | G548A- | VMNLITTCAFAANNIRNIIDPEQIKDILGKDDFIRSLKKDAFNKHIYGLFFP |
| | W621Q- | VGAEMIEPVSLWAMRGAVQQIRNNVAHYKKDALATIFNITKFEYPGAEN |
| | N722R- | ILFSNTIYKQFFEKELEQLPEAFALQLKSGGVLAQYGFDQLKFLLDVVKF |
| | A737S- | HLCRTTVPFAPGFKKVMKGGTGYQNSKQDESFYNLELTAYQLKDDFSEE |
| | W765N- | AWSARYFLLKIIYNYLFLPEFINNDKVFADTVAWLLGKNRQQAQSSKNK |
| | D800R- | NAYAFADIRLMESSESITDYMAYVQSQQIMEENKKEDDKKEDTRINFEK |
| | S807R- | FVLQLFVKGFDTFMKQSAYQFINTPQPQINPTDTNQQQADKLKALEKKIC |
| | S819K- | PFCHIQNNKIDPAKDEQIAFYVYCKLLDARHLSTLRNELIKYRASSESELFI |
| | S827K- | HLTEIIELCLLHADNVPADYNQIYKNKEQCLQRLQPFIAENSDYTNWGDL |
| | N871K- | YVQTDRETPVVHRGIELSVKYGTAKLLEKLIAKDAKFKITENEFDLWHN |
| | D974Y- | ARATIEAMIKKREEYHNLWLEAKEKDDKEKKTRIREKNNYARRFVETK |
| | H1020R- | GEDYIDICDYIDRYNWLNNKLHFEHLKSLQNITIEMLGRMAGFVVLWER |
| | I1064K | DFQYLDQQRVKQQGGALMDFSHGVPKYEELKPHYAYFKRLFLCNNYSD |
| | | TRNHIAHFNYLTKSAANYSLIDLINQLRELLRYDRKLKNAVAKTFIDLFD |
| | | KHGMVLKLKFDSQMHQLQVGSITSKKIFHLGTKKESDNIYTFQVPTAYC |
| | | NMCKQLLEMKN |
| 278 | H106Q- | MRVSKVKVTVNNKDTMVLMHRSAEKGALIYDTIDKKDRTEEIIPQKKQ |
| | H139Q- | KSFELSIQNKTLLRYEAIKKQKLDRSKYKVVEDIFKYRKLPAKITLEDILP |
| | D184E- | FLNHKFQLPVKYQKEGQWVQFELSTCILQAVEHQNLDELQPYYDWSKW |
| | A262T- | YIDDKSGKLKKSITNNHIQIGDGESKRQKALLMWETEFTDKGELDLETY |
| | M311V- | HEKFIIDTLAASLRSVEKGELRDNGTPKETNSYHRNLKKVLQNHQALIFG |
| | K318Q- | RRETPNQENRDNKQLTIYNLEVVKYLEHYFPIKSNVRKNSAADIVYYLKS |
| | S352C- | ETIKSTVTHQLENAVRANLLRQGKYQHHDLKADATSENLTNLKRDEAF |
| | V372L- | VMNLITTCAFAANNIRNIIDPEQIKDILGKDDFIRSLKKDAFNKHIYGLFFP |
| | V501T- | VGAEMIEPVSLWAMRGAVQQIRNNVAHYKKDALATIFNITKFEYPGAEN |
| | G548A- | ILFSNTIYKQFFEKELEQLPEAFALQLKSGGVLAQYGFDQLKFLLDVVKF |
| | T726Q- | HLCRTTVPFAPGFKKVMKGGTGYQNSKQDESFYNLELTAYQLKDDFSEE |
| | N722R- | AWSARYFLLKIIYNYLFLPEFTNNDKVFADTVAWLLGKNRQQAQSSKNK |
| | A737S- | NAYAFADIRLMESSESITDYMAYVQSQWIMEENKKEDDKKEDTRINFEK |
| | W765N- | FVLQLFVKGFDTFMKQSAYQFINTPQPQINPTDTNQQQADKLKALEKKIC |
| | S807R- | PFCHIQNNKIDPAKDEQIAFYVYCKLLDARHLSQLRNELIKYRASSESELF |
| | S819K- | IHLTEIIELCLLHADNVPADYNQIYKNKEQCLQRLQPFIAENSDYTNWGD |
| | N871K- | LYVQTDDETPVVHRGIELSVKYGTAKLLEKLIASDAKFKITENEFDLWHN |
| | D974Y- | ARATIEAMIKKREEYHNLWLEAKEKDDKEKKTRIREKNNYARRFVETK |
| | T1032V- | GEDYIDICDYIDRYNWLNNKLHFEHLKSLQNITIEMLGRMAGFVVLWER |
| | H1020R- | DFQYLDQQRVKQQGGALMDFSHGVPKYEELKPHYAYFKRLFLCNNYSD |
| | I1064K | TRNHIAHFNYLTKSAANYSLIDLINQLRELLRYDRKLKNAVAKVFIDLFD |
| | | KHGMVLKLKFDSQMHQLQVGSITSKKIFHLGTKKESDNIYTFQVPTAYC |
| | | NMCKQLLEMKN |
| 279 | H139Q- | MRVSKVKVTVNNKDTMVLMHRSAEKGALIYDTIDKKDRTEEIIPQKKQ |
| | G154K- | KSFELSIQNKTLLRYEAIKKQKLDRSKYKVVEDIFKYRKLPAKITLEDILP |
| | D184E- | FLNHKFHLPVKYQKEGQWVQFELSTCILQAVEHQNLDELQPYYDWSKW |
| | M311V- | YINDKSKKLKKSITNNHIQIGDGESKRQKALLMWETEFTDKGELDLETY |
| | K318Q- | HEKFIIDTLAASLRSVEKGELRDNGTPKETNSYHRNLKKVLQNHQALIFG |
| | S352C- | RRETPNQENRDNKQLAIYNLEVVKYLEHYFPIKSNVRKNSAADIVYYLK |
| | V372L- | SETIKSTVTHQLENAVRANLLRQGKYQHHDLKADATSENLTNLKRDEAF |
| | V501T- | VMNLITTCAFAANNIRNIIDPEQIKDILGKDDFIRSLKKDAFNKHIYGLFFP |
| | G548A- | VGAEMIEPVSLWAMRGAVQQIRNNVAHYKKDALATIFNITKFEYPGAEN |
| | N722R- | ILFSNTIYKQFFEKELEQLPEAFALQLKSGGVLAQYGFDQLKFLLDVVKF |
| | A737S- | HLCRTTVPFAPGFKKVMKGGTGYQNSKQDESFYNLELTAYQLKDDFSEE |
| | W765N- | AWSARYFLLKIIYNYLFLPEFTNNDKVFADTVAWLLGKNRQQAQSSKNK |
| | D800R- | NAYAFADIRLMESSESITDYMAYVQSQWIMEENKKEDDKKEDTRINFEK |
| | S807R- | FVLQLFVKGFDTFMKQSAYQFINTPQPQINPTDTNQQQADKLKALEKKIC |
| | S819K- | PFCHIQNNKIDPAKDEQIAFYVYCKLLDARHLSTLRNELIKYRASSESELFI |
| | N871K- | HLTEIIELCLLHADNVPADYNQIYKNKEQCLQRLQPFIAENSDYTNWGDL |
| | D974Y- | YVQTDRETPVVHRGIELSVKYGTAKLLEKLIASDAKFKITENEFDLWHN |
| | H1020R- | ARATIEAMIKKREEYHNLWLEAKEKDDKEKKTRIREKNNYARRFVETK |
| | I1064K | GEDYIDICDYIDRYNWLNNKLHFEHLKSLQNITIEMLGRMAGFVVLWER |
| | | DFQYLDQQRVKQQGGALMDFSHGVPKYEELKPHYAYFKRLFLCNNYSD |
| | | TRNHIAHFNYLTKSAANYSLIDLINQLRELLRYDRKLKNAVAKTFIDLFD |
| | | KHGMVLKLKFDSQMHQLQVGSITSKKIFHLGTKKESDNIYTFQVPTAYC |
| | | NMCKQLLEMKN |
| 280 | H106Q- | MRVSKVKVTVNNKDTMVLMHRSAEKGALIYDTIDKKDRTEEIIPQKKQ |
| | H139Q- | KSFELSIQNKTLLRYEAIKKQKLDRSKYKVVEDIFKYRKLPAKITLEDILP |
| | G154K- | FLNHKFQLPVKYQKEGQWVQFELSTCILQAVEHQNLDELQPYYDWSKW |
| | D184E- | YINDKSKKLKKSITNNHIQIGDGESKRQKALLMWETEFTDKGELDLETY |
| | M311V- | HEKFIIDTLAASLRSVEKGELRDNGTPKETNSYHRNLKKVLQNHQALIFG |
| | K318Q- | RRETPNQENRDNKQLAIYNLEVVKYLEHYFPIKSNVRKNSAADIVYYLK |
| | S352C- | SETIKSTVTHQLENAVRANLLRQGKYQHHDLKADATSENLTNLKRDEAF |
| | V372L- | VMNLITTCAFAANNIRNIIDPEQIKDILGKDDFIRSLKKDAFNKHIYGLFFP |
| | V501T- | VGAEMIEPVSLWAMRGAVQQIRNNVAHYKKDALATIFNITKFEYPGAEN |
| | G548A- | ILFSNTIYKQFFEKELEQLPEAFALQLKSGGVLAQYGFDQLKFLLDVVKF |
| | N722R- | HLCRTTVPFAPGFKKVMKGGTGYQNSKQDESFYNLELTAYQLKDDFSEE |
| | A737S- | AWSARYFLLKIIYNYLFLPEFTNNDKVFADTVAWLLGKNRQQAQSSKNK |
| | A762G- | NAYAFADIRLMESSESITDYMAYVQSQWIMEENKKEDDKKEDTRINFEK |
| | W765N- | FVLQLFVKGFDTFMKQSAYQFINTPQPQINPTDTNQQQADKLKALEKKIC |
| | S807R- | PFCHIQNNKIDPAKDEQIAFYVYCKLLDARHLSTLRNELIKYRASSESELFI |
| | S819K- | HLTEIIELCLLHADNVPGDYNQIYKNKEQCLQRLQPFIAENSDYTNWGDL |
| | N871K- | YVQTDDETPVVHRGIELSVKYGTAKLLEKLIASDAKFKITENEFDLWHN |
| | D974Y- | ARATIEAMIKKREEYHNLWLEAKEKDDKEKKTRIREKNNYARRFVETK |
| | H1020R- | GEDYIDICDYIDRYNWLNNKLHFEHLKSLQNITIEMLGRMAGFVVLWER |
| | I1064K | DFQYLDQQRVKQQGGALMDFSHGVPKYEELKPHYAYFKRLFLCNNYSD |
| | | TRNHIAHFNYLTKSAANYSLIDLINQLRELLRYDRKLKNAVAKTFIDLFD |
| | | KHGMVLKLKFDSQMHQLQVGSITSKKIFHLGTKKESDNIYTFQVPTAYC |
| | | NMCKQLLEMKN |
| 281 | H139Q- | MRVSKVKVTVNNKDTMVLMHRSAEKGALIYDTIDKKDRTEEIIPQKKQ |
| | D184E- | KSFELSIQNKTLLRYEAIKKQKLDRSKYKVVEDIFKYRKLPAKITLEDILP |
| | M311V- | FLNHKFHLPVKYQKEGQWVQFELSTCILQAVEHQNLDELQPYYDWSKW |
| | K318Q- | YINDKSGKLKKSITNNHIQIGDGESKRQKALLMWETEFTDKGELDLETY |
| | S352C- | HEKFIIDTLAASLRSVEKGELRDNGTPKETNSYHRNLKKVLQNHQALIFG |
| | V372L- | RRETPNQENRDNKQLAIYNLEVVKYLEHYFPIKSNVRKNSAADIVYYLK |
| | V501T- | SETIKSTVTHQLENAVRANLLRQGKYQHHDLKADATSENLTNLKRDEAF |
| | E543L- | VMNLITTCAFAANNIRNIIDPEQIKDILGKDDFIRSLKKDAFNKHIYGLFFP |
| | G548A- | VGAEMIEPVSLWAMRGAVQQIRNNVAHYKKDALATIFNITKFEYPGAEN |
| | T726Q- | ILFSNTIYKQFFEKELEQLPEAFALQLKSGGVLAQYGFDQLKFLLDVVKF |
| | N722R- | HLCRTTVPFAPGFKKVMKGGTGYQNSKQDESFYNLELTAYQLKDDFSLE |
| | A737S- | AWSARYFLLKIIYNYLFLPEFTNNDKVFADTVAWLLGKNRQQAQSSKNK |
| | W765N- | NAYAFADIRLMESSESITDYMAYVQSQWIMEENKKEDDKKEDTRINFEK |
| | S807R- | FVLQLFVKGFDTFMKQSAYQFINTPQPQINPTDTNQQQADKLKALEKKIC |
| | S819K- | PFCHIQNNKIDPAKDEQIAFYVYCKLLDARHLSQLRNELIKYRASSESELF |
| | N871K- | IHLTEIIELCLLHADNVPADYNQIYKNKEQCLQRLQPFIAENSDYTNWGD |
| | D974Y- | LYVQTDDETPVVHRGIELSVKYGTAKLLEKLIASDAKFKITENEFDLWHN |
| | I1064K | ARATIEAMIKKREEYHNLWLEAKEKDDKEKKTRIREKNNYARRFVETK |
| | | GEDYIDICDYIDRYNWLNNKLHFEHLKSLQNITIEMLGRMAGFVVLWER |
| | | DFQYLDQQRVKQQGGALMDFSHGVPKYEELKPHYAYFKRLFLCNNYSD |
| | | TRNHIAHFNYLTKSAANYSLIDLINQLRELLHYDRKLKNAVAKTFIDLFD |
| | | KHGMVLKLKFDSQMHQLQVGSITSKKIFHLGTKKESDNIYTFQVPTAYC |
| | | NMCKQLLEMKN |
| 282 | H139Q- | MRVSKVKVTVNNKDTMVLMHRSAEKGALIYDTIDKKDRTEEIIPQKKQ |
| | G154K- | KSFELSIQNKTLLRYEAIKKQKLDRSKYKVVEDIFKYRKLPAKITLEDILP |
| | D184E- | FLNHKFHLPVKYQKEGQWVQFELSTCILQAVEHQNLDELQPYYDWSKW |
| | M311V- | YINDKSKKLKKSITNNHIQIGDGESKRQKALLMWETEFTDKGELDLETY |
| | K318Q- | HEKFIIDTLAASLRSVEKGELRDNGTPKETNSYHRNLKKVLQNHQALIFG |
| | S352C- | RRETPNQENRDNKQLAIYNLEVVKYLEHYFPIKSNVRKNSAADIVYYLK |
| | V372L- | SETIKSTVTHQLENAVRANLLRQGKYQHHDLKADATSENLTNLKRDEAF |
| | V501T- | VMNLITTCAFAANNIRNIIDPEQIKDILGKDDFIRSLKKDAFNKHIYGLFFP |
| | E543L- | VGAEMIEPVSLWAMRGAVQQIRNNVAHYKKDALATIFNITKFEYPGAEN |
| | G548A- | ILFSNTIYKQFFEKELEQLPEAFALQLKSGGVLAQYGFDQLKFLLDVVKF |
| | T726Q- | HLCRTTVPFAPGFKKVMKGGTGYQNSKQDESFYNLELTAYQLKDDFSLE |
| | N722R- | AWSARYFLLKIIYNYLFLPEFTNNDKVFADTVAWLLGKNRQQAQSSKNK |
| | A737S- | NAYAFADIRLMESSESITDYMAYVQSQWIMEENKKEDDKKEDTRINFEK |
| | W765N- | FVLQLFVKGFDTFMKQSAYQFINTPQPQINPTDTNQQQADKLKALEKKIC |
| | S807R- | PFCHIQNNKIDPAKDEQIAFYVYCKLLDARHLSQLRNELIKYRASSESELF |
| | S819K- | IHLTEIIELCLLHADNVPADYNQIYKNKEQCLQRLQPFIAENSDYTNWGD |
| | N871K- | LYVQTDDETPVVHRGIELSVKYGTAKLLEKLIASDAKFKITENEFDLWHN |
| | D974Y- | ARATIEAMIKKREEYHNLWLEAKEKDDKEKKTRIREKNNYARRFVETK |
| | H1020R- | GEDYIDICDYIDRYNWLNNKLHFEHLKSLQNITIEMLGRMAGFVVLWER |
| | I1064K | DFQYLDQQRVKQQGGALMDFSHGVPKYEELKPHYAYFKRLFLCNNYSD |
| | | TRNHIAHFNYLTKSAANYSLIDLINQLRELLRYDRKLKNAVAKTFIDLFD |
| | | KHGMVLKLKFDSQMHQLQVGSITSKKIFHLGTKKESDNIYTFQVPTAYC |
| | | NMCKQLLEMKN |

TABLE 2 provides illustrative sequences of exemplary heterologous polypeptide modifications of effector protein(s) that are useful in the compositions, systems and methods described herein.

TABLE 2
Sequences of Exemplary Heterologous Polypeptide
Modifications of Effector Protein(s)

| SEQ | | |
|---|---|---|
| ID | | |
| NO: | Description | Sequence* |
| 16 | NLS | KR(K/R)R |
| 17 | NLS | (P/R)XXKR(^{∧}D/E)(K/R) |
| 18 | NLS | KRX(W/F/Y)XXAF |
| 19 | NLS | (R/P)XXKR(K/R)(^{∧}D/E) |
| 20 | NLS | LGKR(K/R)(W/F/Y) |
| 21 | NLS | KRX₁₀K(K/R)(K/R) |
| 22 | EEP | GLFXALLXLLXSLWXLLLXA |
| 23 | NLS | K(K/R)RK |
| 24 | NLS | KRX₁₁K(K/R)(K/R) |
| 25 | NLS | KRX₁₂K(K/R)(K/R) |
| 26 | NLS | KRX₁₀K(K/R)X(K/R) |
| 27 | NLS | KRX₁₁K(K/R)X(K/R) |
| 28 | NLS | KRX₁₂K(K/R)X(K/R) |
| 29 | NLS | APKKKRKVGIHGVPAA |

*wherein X is independently any naturally occurring amino acid; and ^{∧}D/E is any naturally occurring amino acid except Asp or Glu

TABLE 3 provides illustrative repeat sequences for use in guide nucleic acids that are useful in the compositions, systems and methods described herein.

TABLE 3
Exemplary Repeat Sequences for use in Guide Nucleic Acids

| Effector | | | |
|---|---|---|---|
| Protein | Effector | Repeat | |
| SEQ ID | Protein | SEQ ID | |
| NO: | ID | NO: | Repeat Sequence (5′→3′), shown as RNA |
| 1 | 2712211 | 30 | GUACAGUAGCCCUACAGAUUCGUAGGGUUCUGUGAC |
| 14 | 2712490 | 30 | GUACAGUAGCCCUACAGAUUCGUAGGGUUCUGUGAC |
| 2 | 2711416 | 31 | GUUUUAGACCCCUGAUAUUUCGCAAGGGUAUAGUAC |
| 6 | 2715166 | 31 | GUUUUAGACCCCUGAUAUUUCGCAAGGGUAUAGUAC |
| 3 | 2711260 | 32 | GUUUCAGACCCCUACCAUUUGGUAAGGGUAUAGUAC |
| 10 | 2724407 | 32 | GUUUCAGACCCCUACCAUUUGGUAAGGGUAUAGUAC |
| 4 | 2712493 | 33 | GCACUACACCCCUGACUUCGGUCGGGGGUCUAAAAC |
| 15 | 2712135 | 33 | GCACUACACCCCUGACUUCGGUCGGGGGUCUAAAAC |
| 5 | 2712234 | 34 | GUAAGACAACCCUACAAACCUGUAGGGUUCUGAGAC |
| 11 | 2712332 | 34 | GUAAGACAACCCUACAAACCUGUAGGGUUCUGAGAC |
| 2 | 2711416 | 35 | GAACUACACCCCUGCGAAAUACGAGGGGUCUAAAAC |
| 6 | 2715166 | 35 | GAACUACACCCCUGCGAAAUACGAGGGGUCUAAAAC |
| 7 | 2712237 | 36 | GUGUCAGACCCCUGUCAGAUAACAGGGGUGUAGUUCC |
| 8 | 2208675 | 37 | GUUGUAACUGCUCUUAUUUUGAAGGGUAAACACAAC |
| 9 | 2725811 | 38 | GUUUCAGACUCCUGACGGAGUGUAGUAGUAACGUUC |
| 3 | 2711260 | 39 | GUACUACACCCCUGGGAUUUCACAGGGGUCUGAAAC |
| 10 | 2724407 | 39 | GUACUACACCCCUGGGAUUUCACAGGGGUCUGAAAC |
| 5 | 2712234 | 40 | GAACUACACCCCUGUAGAAUUACAGGGGUCUAAAAC |
| 11 | 2712332 | 40 | GAACUACACCCCUGUAGAAUUACAGGGGUCUAAAAC |
| 12 | 2708655 | 41 | UGUAGUUUUACAUGGGUGUAAAAC |
| 13 | 2725856 | 42 | GAACUAAACCCCUGCGGAUAUGCAGGGGUCUGAAAC |
| 8 | 2208675 | 42 | GAACUAAACCCCUGCGGAUAUGCAGGGGUCUGAAAC |
| 14 | 2712490 | 43 | GUUCUAUACCCCUGUGAUUUACCAGGGGUCUGAAACA |
| 4 | 2712493 | 44 | GGACUACACCUUGGCAGUAAUGCUGAGGUCUGAAAC |
| 15 | 2712135 | 44 | GGACUACACCUUGGCAGUAAUGCUGAGGUCUGAAAC |
| 93 | 3610137 | 45 | UGUUGUAAAUGCCCUAAUUUUGAAAGGUAAACACAAC |
| 12 | 2708655 | 46 | GAACGUUACUACUACACUCCGUCAGGAGUCUGAAAC |
| 2 | 2711416 | 47 | CACCCCUGCGAAAUACGAGGGGUCUAAAAC |
| 5 | 2712234 | 48 | CACCCCUGUAGAAUUACAGGGGUCUAAAAC |
| 7 | 2712237 | 48 | CACCCCUGUAGAAUUACAGGGGUCUAAAAC |
| 68 | 2108577 | 48 | CACCCCUGUAGAAUUACAGGGGUCUAAAAC |
| 10 | 2724407 | 49 | GACCCCUACCAUUUGGUAAGGGUAUAGUAC |
| 1 | 2712211 | 50 | GUUCUAUACCCCUGUGAUUUACCAGGGGUCUGAAAC |
| 7 | 2712237 | 52 | GUGUCAGACCCCUGUCAGAUAACAGGGGUGUAGUUC |
| 94 | | 53 | GUUUUAGACCCCUUCGUUUUUGGGGUUAUCUAUAUC |
| 95 | | 53 | GUUUUAGACCCCUUCGUUUUUGGGGUUAUCUAUAUC |
| 96 | | 53 | GUUUUAGACCCCUUCGUUUUUGGGGUUAUCUAUAUC |
| 97 | | 53 | GUUUUAGACCCCUUCGUUUUUGGGGUUAUCUAUAUC |
| 97 | | 54 | GAUAUAGAUAACCCCAAAAACGAAGGGGACUAAAAC |
| 98 | | 55 | AGUUUUAGUCCCCUUUGAUAUUGG |
| 99 | | 55 | AGUUUUAGUCCCCUUUGAUAUUGG |
| 98 | | 56 | UUUUAGUCCCCUUCAUUUUUGGGGUGCUCUAAAUC |
| 99 | | 56 | UUUUAGUCCCCUUCAUUUUUGGGGUGCUCUAAAUC |
| 90 | | 56 | UUUUAGUCCCCUUCAUUUUUGGGGUGCUCUAAAUC |
| 91 | | 56 | UUUUAGUCCCCUUCAUUUUUGGGGUGCUCUAAAUC |
| 92 | | 56 | UUUUAGUCCCCUUCAUUUUUGGGGUGCUCUAAAUC |
| 69* | 2132026 | 57 | GGCCACCCCAAAAAUGAAGGGGACUAAAACA |
| 9 | 2725811 | 58 | GUUUUAGAACUGUGUAGUUUUACAUGGGUGUAAAAC |
| 68 | 2108577 | 59 | AAUGUGAAUGAUCCCAAUAUUGAAGGGAACUACAAC |
| 69* | 2132026 | 60 | AUUGUGGAUUAUCCCAAUAUUGAAGGGAACUACAAC |
| 70 | 3016461 | 61 | GAACUAUAGUAGUGUGAAUUAACACUACUCUAAAAC |
| 71 | 3024180 | 62 | GUUUUAGACUACUAGCAUUCUUUAUUGUUCUAAAAC |
| 72 | 3028750 | 63 | AUUACUGCACAGGGGAUUCUAGAGC |
| 73 | 3028811 | 64 | GGGAACUACACCCCUGCUAACCUGCAGGGGUCUGAAAC |
| 74 | 3035346 | 65 | GUUUUAGACCCAUGUUAUUUAGCAUGGGUGUAGUUC |
| 75 | 3037546 | 66 | GAACUACACCCAUGCUAAAUAACAUGGGUCUAAAAC |
| 76 | 3040041 | 67 | GUGUCAGACCCCUCAAGUUUCUGAGGGGUAUAGUAC |
| 69* | 2132026 | 283 | GAUUAUCCCAAUAUUGAAGGGAACUACAAC |

* = Variants of this effector protein as described in TABLE 1.1 and TABLE 1.2 can also use the repeat sequence associated with the effector protein in the compositions, systems and methods described herein.

TABLE 4 provides illustrative target nucleic acids that are useful in the compositions, systems and methods described herein.

TABLE 4
Exemplary Target Nucleic Acids
Exemplary Targets

AAVS1, ABCA4, ABCB11, ABCC8, ABCD1, ABCG5, ABCG8, ACAD9, ACADM, ACADVL, ACAT1,
ACTA1, ACOX1, ACSF3, ADA, ADAMTS2, ADGRG1, AGA, AGL, AGPS, AGXT, AHI1, AIRE,
ALDH3A2, ALDOB, ALG6, ALK, ALKBH5, ALMS1, ALPL, AMRC9, AMT, ANAPC10, ANAPC11,
ANGPTL3, ANGPTL4, ANGPTL7, APC, Apo(a), APOCIII, APOE, APOEε4, APOL1, APP, AQP2, AR,
ARFRP1, ARG1, ARH, ARL13B, ARL6, ARSA, ARSB, ASL, ASNS, ASPA, ASS1, ATM, ATP6V1B1,
ATP7A, ATP7B, ATRX, ATXN1, ATXN10, ATXN2, ATXN3, ATXN7, ATXN8OS, AXIN1, AXIN2, B2M,
BACE-1, BAK1, BAP1, BARD1, BAX2, BBS1, BBS10, BBS12, BBS2, BCKDHA, BCKDHB, BCL2L2,
BCS1L, BEST1, Betaglobin gene, BLM, BMPR1A, BRAF, BRAFV600E, BRCA1, BRCA2, BRIP1,
BSND, C9orf72, CA4, CACNA1A, CAH1, CAPN3, CASR, CBS, CCNB1 CC2D2A, CCR5, CD1, CD2,
CD3, CD3D, CD3Z, CD4, CD5, CD6, CD7, CD8A, CD8B, CD9, CD14, CD18, CD19, CD21, CD22,
CD23, CD27, CD28, CD30, CD33, CD34, CD36, CD38, CD40, CD40L, CD44, CD46, CD47, CD48,
CD52, CD55, CD57, CD58, CD59, CD68, CD69, CD72, CD73, CD74, CD79A, CD80, CD81, CD83,
CD84, CD86, CD90, CD93, CD96, CD99, CD100, CD123, CD160, CD163, CD164, CD164L2,
CD166, CD200, CD204, CD207, CD209, CD226, CD244, CD247, CD274, CD276, CD300, CD320,
CDC73, CDH1, CDH23, CDK11, CDK4, CDKN1A, CDKN1B, CDKN1C, CDKN2A, CDKN2B,
CEBPA, CELA3B, CEP290, CERKL, CFB, CFTR, CHCHD10, CHEK2, CHM, CHRNE, CIDEB,
CIITA, CLN3, CLN5, CLN6, CLN8, CLRN1, CLTA, CMT1A, CNBP, CNGB1, CNGB3, COL1A1,
COL1A2, COL27A1, COL4A3, COL4A4, COL4A5, COL6A1, COL6A2, COL6A3, COL7A1, CPS1,
CPT1A, CPT2, CRB1, CREBBP, CRX, CRYAA, CTNNA1, CTNNB1, CTNND2, CTNS, CTSK,
CXCL12, CYBA, CYBB, CYP11B1, CYP11B2, CYP17A1, CYP19A1, CYP21A2, CYP27A1, DBT, DCC,
DCLRE1C, DERL2, DFNA36, DFNB31, DGAT2, DHCR7, DHDDS, DICER1, DIS3L2, DLD, DMD,
DMPK, DNAH5, DNAI1, DNAI2, DNM2, DNMT1, DPC4, DUX4, DYSF, EDA, EDN3, EDNRB,
EGFR, EIF2B5, EMC2, EMC3, EMD, EMX1, EN1, EPCAM, ERCC6, ERCC8, ESCO2, ETFA,
ETFDH, ETHE1, EVC, EVC2, EYS, F5, F9, FXI, FAH, FAM161A, FANCA, FANCB, FANCC,
FANCD1, FANCD2, FANCE, FANCF, FANCG, FANCI, FANCJ, FANCL, FANCM, FANCN, FANCP,
FANCS, FBN1, FGF14, FGFR2, FGFR3, FGA, FGB, FGG, FH, FHL1, FIX, FKRP, FKTN, FLCN,
FMR1, FOXP3, FSCN2, FSHD1, FUS, FUT8, FVIII, FXII, FXN, G6PC, GAA, GALC, GALK1, GALT,
GAMT, GATA2, GATA-4, GBA, GBE1, GCDH, GCGR, GDNF, GFAP, GFM1, GHR, GJB1, GJB2,
GLA, GLB1, GLDC, GLE1, GNE, GNPTAB, GNPTG, GNS, GPAM, GPC3, GPR98, GREM1, GRHPR,
GRIN2B, H2AFX, H2AX, HADHA, HAX1, HBA1, HBA2, HBB, HBV cccDNA, HER2, HEXA, HEXB,
HFE, HGSNAT, HLCS, HMGCL, HAO1, HOGA1, HOXB13, HPRPF3, HPRT1, HPS1, HPS3, HRAS,
HRD1, HSD3B2, HSD17B4, HSD17B13, HTT, HUS1, HYAL1, HYLS1, IDS, IDUA, IFITM5, IFN,
IFN-γ, IKBKAP, IL2RG, IL7R, IMPDH1, INPP5E, IRF4, ITGB2, ITPR1, IVD, JAG1, JAK1, JAK3,
KCNC3, KCND3, KCNJ11, KLKB1, KLHL7, KRAS, LAMA1, LAMA2, LAMA3, LAMB3, LAMC2,
LCA5, LDHA, LDLR, LDLRAP1, LHX3, LIFR, LIPA, LMNA, LMOD3, LOR, LOXHD1, LPA, LPL,
LRAT, LRP6, LRPPRC, LRRK2, MADR2, MAN2B1, MAPT, MARC1, MAX, MCM6, MCOLN1,
MECP2, MED17, MEFV, MEN1, MERTK, MESP2, MET, METex14, MFN2, MFSD8, MIA3, MITF,
MKL2, MKS1, MLC1, MLH1, MLH3, MMAA, MMAB, MMACHC, MMADHC, MMD, MPI, MPL,
MPV17, MSH2, MSH3, MSH6, MTHFD1L, MTHFR, MTM1, MTRR, MTTP, MUT, MUTYH, MYC,
MYH7, MYO7A, MYOC, NAGLU, NAGS, NAV1.7, NBN, NDRG1, NDUFAF5, NDUFS6, NEB, NF1,
NF2, NKX2-5, NOG, NOTCH1, NOTCH2, NPC1, NPC2, NPHP1, NPHS1, NPHS2, NRAS, NR2E3,
NTHL1, NTRK, NTRK1, OAT, OCT4, OFD1, OPA3, OTC, PAH, PALB2, PAQR8, PAX3, PC, PCCA,
PCCB, PCDH15, PCSK9, PD1, PDCD1, PDE6B, PDGFRA, PDHA1, PDHB, PEX1, PEX10, PEX12,
PEX13, PEX14, PEX16, PEX19, PEX2, PEX26, PEX3, PEX5, PEX6, PEX7, PFKM, PHGDH,
PHOX2B, PKD1, PKD2, PKHD1, PKK, PLEKHG4, PMM2, PMP22, PMS1, PMS2, PNPLA3,
POLD1, POLE, POMGNT1, POT1, POU5F1, PPM1A, PPP2R2B, PPT1, PRCD, PRKAG2,
PRKAR1A, PRKCG, PRNP, PROM1, PROP1, PRPF31, PRPF8, PRPH2, PRPS1, PSAP, PSD3,
PSD95, PSEN1, PSEN2, PSRC1, PTCH1, PTEN, PTS, PUS1, PYGM, RAB23, RAD50, RAD51C,
RAD51D, RAG1, RAG2, RAPSN, RARS2, RB1, RDH12, RECQL4, RET, RHO, RICTOR, RMRP, ROS1,
RP1, RP2, RPE65, RPGR, RPGRIP1L, RPL32P3, RPTOR, RS1, RTCA, RTEL1, RUNX1, SACS,
SAMHD1, SCN1A, SCN2A, SDHA, SDHAF2, SDHB, SDHC, SDHD, SEL1L, SEPSECS, SERPINA1,
SERPINC1, SERPING1, SGCA, SGCB, SGCG, SGSH, SIRT1, SLC12A3, SLC12A6, SLC17A5,
SLC22A5, SLC25A13, SLC25A15, SLC26A2, SLC26A4, SLC35A3, SLC35B4, SLC37A4, SLC39A4,
SLC4A11, SLC6A8, SLC7A7, SMAD3, SMAD4, SMARCA4, SMARCAL1, SMARCB1, SMARCE1,
SMN1, SMPD1, SNAI2, SNCA, SNRNP200, SOD1, SOX10, SPARA7, SPTBN2, STAR, STAT3, STK11,
SUFU, SUMF1, SYNE1, SYNE2, SYS1, TARDBP, TAT, TBK1, TBP, TCF4, TCIRG1, TCTN3,
TECPR2, TERC, TERT, TFR2, TGFBR2, TGM1, TH, TLE3, TMEM127, TMEM138, TMEM216,
TMEM43, TMEM67, TMPRSS6, TNNI2, TNNT1, TNNT3, TOP1, TOPORS, TP53, TPM2, TPM3,
TPP1, TRAC, TRMU, TSC1, TSC2, TSFM, TSPAN14, TTBK2, TTC8, TTPA, TTR, TULP1, TYMP,
UBE2G2, UBE2J1, UBE3A, USH1C, USH1G, USH2A, VEGF, VHL, VPS13A, VPS13B, VPS35,
VPS45, VRK1, VSX2, VWF, WAS, WDR19, WDR48, WNT10A, WRN, WS2B, WS2C, WT1, XPA, XPC,
XPF, XRCC3, YAP1, ZAC1, ZEB1, ZFYVE26, and ZNF423

TABLE 4.1 provides illustrative genes that produce target RNAs that are useful in the compositions, systems and methods described herein.

TABLE 4.1
Exemplary Target Nucleic Acids
Exemplary Targets

ABCA9, ABCB10, ABCC1, ABCC4, ABHD2, ABTB1, ACACA, ACAP2, ACP6, ACTN4, ACVR2A,
ADAM9, ADAMTS13, ADAMTS6, ADCY1, ADD1, AFAP1, AFF2, AGFG1, AGXT2L2, AHI1, AKT2,
AKT3, ALDH1A1, ALDH1A2, AMOTL1, AMOTL1L, ANAPC7, ANKLE2, ANKRD12, ANKRD17,
ANKRD36, ANO6, ANRIL, ANXA1, ANXA2, AP4E1, APP, ARAP2, ARFIP2, ARHGAP26, ARHGAP5,
ARHGEF12, ARHGEF26, ARID1A, ARIH1, ARL8B, ARPC1B, ASAP1, ASAP2, ASH2L, ASPH,
ATG4C, ATIC, ATP2A2, ATP2B1, ATP5SL, ATP8A2, ATP8B1, ATP9B, ATRNL1, ATXN10, ATXN7,
AXL, AZIN1, B3GALNT2, B3GNTL1, B4GALT2, BACH2, BANP, BARD41, BASP1, BCAR3, BCL11B,
BEND6, BGN, BICD2, BIRC6, BNC2, BPTF, BRAF, BRIP1, BTBD7, C11orf73, C11orf80, C14orf118,
C15orf41, C17orf81, C2orf43, C3P1, C6orf132, CACNA2D1, CACNB4, CADPS2, CAMTA1, CANX,
CBFB, CCDC66, CCNB1, CCNYL1, CCS, CCT3, CCT4, CD44, CD99, CDC27, CDK13, CDK14,
CDK6, CDKN2B-AS1, CDKN3, CDR1, CDR1as, CDYL, CECR2, CELSR1, CEP128, CEP192, CFH,
CGNL1, CHCHD10, CHD7, CHD9, CHMP5, CHTOP, CLINT1, CLIP1, CLIP2, CMTM3, CNIH4,
COL1A1, COL1A2, COL5A1, CORO1C, CR1, CRIM1, CRKL, CSE1L, CSF2RB, CSNK1G1, CSPP1,
CSRP1, CTBP2, CTDP1, CTNNA2, CTNNB1, CTSB, CUL1, CUL3, CUL5, CXXC5, DAZAP1, DCAF6,
DCAF8, DCUN1D4, DDX42, DEF6, DENND1A, DENND2A, DENND4C, DGCR8, DHCR24, DHPS,
DICER1, DLEU2, DLST, DNA2, DNAH14, DNAJC6, DNMT1, DOCK1, DONSON, DOPEY2, DPYD,
DROSHA, DTNA, DYM, DYNC1H1, DYNLL1, EEFSEC, EFCAB11, EFCAB2, EGFR, EHD2, EIF4B,
EIF4G2, EIF4G3, EIF6, ELK4, ELMO1, ELP3, EML4-ALK, EML5, EPHB4, EPS15, EPSTI1, ERC1,
ERN1, ESRP1, ETFA, EXOC6B, FADS2, FAF1, FAM114A2, FAM120A, FAM120B, FAM126A,
FAM169A, FAM188A, FAM49B, FAM53B, FAM64A, FARSA, FASN, FAT1, FBLIM, FBXO7, FBXW7,
FGFR1, FGFR3, FGGY, FIP1 L1, FKBP8, FLNA, FN1, FNDC3B, FOXM1, FOXO3, FOXRED1,
FSTL1, FTO, FURIN, FUT8, GADD45A, GDA, GFRA1, GLI2, GLIS3, GNB4, GNG12, GRB10,
GSDMB, GSK3B, GSPT1, GTF3C5, GTPBP3, GUSBP11, HABP4, HAS2, HEATR2, HECW2, HERC2,
HGD, HIAT1, HIP1, HIPK3, HLA-C, HMGCS1, HNRNPF, HOMER1, HSP90AA1, HSP90B1, HSPG2,
IARS, IBTK, IFF01, IFNGR2, IGF1R, IGF2BP3, INSR, IPO11, IQCK, IRAK1, IRAK3, ITCH, ITGA7,
ITGA8, ITGB6, JMJD1C, KATNAL1, KCNH1, KCNN2, KCNQ5, KIAA0355, KIAA0907, KIAA1244,
KIF4A, KLF6, KLHDC10, KLHL20, KMT2E, KPNA6, KPNB1, KRT75, KRT81, LAMA3, LAMC1,
LAMC2, LAMP1, LAMP2, LAPTM4A, LARP1B, LARP4, LCOR, LDHA, LDLRAD3, LIN52,
LINC00340, LOC100507412, LPXN, LRIG1, LRP6, LRPPRC, LYN, MAN1A2, MAN2B2, MAP2K4,
MAP4K3, MAPK1, MAPK8, MAPK9, MASP1, MCTP1, MED12L, MED13, MED24, MEMO1,
METTL3, MFN2, MFSD2A, MLLT10, MMP1, MMP9, MORC3, MPPED2, MTG1, MTO1, MTRF1,
MYLK, MYO9A, MYO9B, N4BP2L2, NAALADL2, NARS, NASP, NAV3, NBAS, NCOA3, NDST1,
NEK6, NEK9, NF1, NFATC3, NFIB, NFIX, NHS, NIN, NISCH, NMRAL1, NNT, NOL10, NOLC1,
NOTCH, NOX4, NPM1, NQO, NRIP, NT5C2, NT5DC2, NT5E, NTNG1, NUP153, NUP160, NUP214,
NuSAP1, OGDH, OLA1, OMA1, ORC4, OSBPL10, OXCT1, PABPC1, PABPN1, PAIP2, PALLD,
PAN3, PAPD4, PAPPA, PAPSS1, PARD3, PAX2, PCDH9, PCNX, PCNXL2, PDE3A, PDE4B,
PDE7B, PDK1, PDXK, PDZD8, PGC, PGM1, PGPEP1, PHACTR2, PHC3, PHDLA1, PICALM,
PIGB, PIGV, PIK3CB, PIK3R1, PIP5K1A, PLCL2, PLEKHA2, PLOD2, PNPT1, POLD1, POLE2,
POMT1, PPARA, PPFIA1, PPP1R12A, PPP1R13L, PRDM2, PRELID2, PRKCB, PRKCH, PRKCI,
PRKDC, PRMT5, PRRC2B, PSD3, PSEN2, PSME4, PTGR1, PTK2, PTN, PTPN22, PTPRA, PUM1,
PVRL3, PVT1, PWP2, R3HCC1l, R3HDM1, RAB2A, RAD23B, RANBP9, RAPGEF5, RBM4, RBMS3,
RCN1, RELL1, REXO2, RFWD2, RFWD3, RFX3, RGNEF, RHCG, RHOBTB3, RHOT1, RIMS1,
RNF10, RNF111, RNF121, RNF13, RNF216, RNF38, ROBO2, RORA, RPA2, RPH3AL, RPPH1,
RPS5, RRP12, RSL1D1, RTN4, SAFB2, SAMD4A, SATB1, SBF1, SCARF1, SCLT1, SCMH1, SDHA,
SEC24A, SEMA4B, SENP5, SERPINB5, SERPINE2, SETD2, SETD3, SH3PXD2A, SHKBP1, SHPRH,
SIPA1L1, SKA3, SKIL, SLAMF6, SLC16A12, SLC25A15, SLC25A25, SLC26A7, SLC30A7, SLC3A2,
SLC6A6, SLC8A1, SLIT3, SMAD1, SMARC1A, SMARCA5, SMO, SMOC1, SMURF2, SMYD4,
SNAP47, SNRK, SNX13, SNX19, SOAT1, SOD2, SOX13, SP100, SPECC1, SPIN1, SPPL3, SPRR1B,
SPTAN1, SRCAP, SRGAP1, ST3GAL3, ST5, STIL, STIM1, STK17A, STK24, STON2, STXBP5L,
SUSD1, SYT15, SYT7, TADA2A, TAF1, TAF15, TATDN3, TBCD, TC2N, TCEA3, TCEB1, TCF25,
TCONS, TFPI, TFRC, TGFBI, THBS2, TIMMDC1, TITIN, TLK1, TMEM189-UBE2V1, TMEM65,
TNS3, TNS4, TOP2A, TP53, TP63, TPM4, TPTE2, TPX2, TRAPPC6B, TRDMT1, TRIM24, TRIM28,
TRIO, TROAP, TSN, TSNAX-DISC1, TTBK2, TTBK2, TTC17, TTLL5, TUBGCP3, TWIST1, TXLNGY,
TXNDC11, UBA2, UBAP2, UBASH3B, UBQLN1, UBR1, UBR5, UBXN7, UGGT2, UNC13C, UNC79,
UNK, USF1, USP10, USP22, USP3, USP9Y, UVRAG, VANGL1, VAPA, VAV2, VCAN, VDAC3, VIL1,
VMA21, VPS13a, VPS33B, VPS37C, VPS45, VRK1, WBSCR17, WDR26, WDR27, WDR37, WDR48,
WDR77, WDR85, XPO1, XPOT, XPR1, YAP1, YEATS2, YES1, ZC3HC1, ZCCHC7, ZDHHC4, ZEB1,
ZFAND3, ZFR, ZKSCAN1, ZMYM2, ZNF148, ZNF292, ZNF483, ZNF536, ZNF608, ZNF609,
ZNF652, ZNF700, ZNF720, ZNF83, and ZRANB1

TABLE 5 provides illustrative diseases or disorders for compositions, systems and methods described herein.

TABLE 5
Diseases or Disorders
Exemplary Diseases and Syndromes

11-hydroxylase deficiency; 17,20-desmolase deficiency; 17-hydroxylase deficiency; 3-
hydroxyisobutyrate aciduria; 3-hydroxysteroid dehydrogenase deficiency; 46,XY gonadal dysgenesis;
AAA syndrome; ABCA3 deficiency; ABCC8-associated hyperinsulinism; aceruloplasminemia;
acromegaly; achondrogenesis type 2; acral peeling skin syndrome; acrodermatitis enteropathica; acute
bacterial infection; adrenocortical micronodular hyperplasia; adrenoleukodystrophies;
adrenomyeloneuropathies; Aicardi-Goutieres syndrome; AIDS; Alagille disease (also called Alagille
Syndrome); Alexander Disease; Alpers syndrome; alpha-1 antitrypsin deficiency (AATD); alpha-
mannosidosis; Alstrom syndrome; Alzheimer's disease; amebic dysentery; amelogenesis imperfecta;
amish type microcephaly; amyotrophic lateral sclerosis (ALS); anaplastic large cell lymphoma;
anauxetic dysplasia; androgen insensitivity syndrome; angiopathic thrombosis; antiphospholipid
syndrome; Antley-Bixler syndrome; APECED; Apert syndrome; aplasia of lacrimal and salivary
glands; arginase-1 deficiency; argininosuccinic aciduria; argininemia; arrhythmogenic right ventricular
dysplasia; Arts syndrome; ARVD2; arylsulfatase deficiency type metachromatic leukodystrophy;
ataxia telangiectasia; atherosclerotic cardiovascular disease; autoimmune lymphoproliferative
syndrome; autoimmune polyglandular syndrome type 1; autosomal dominant anhidrotic ectodermal
dysplasia; autosomal dominant deafness; autosomal dominant polycystic kidney disease; autosomal
recessive microtia; autosomal recessive renal glucosuria; autosomal visceral heterotaxy; babesiosis;
bacterial vaginosis; balantidial dysentery; Bardet-Biedl syndrome; Bartter syndrome; basal cell nevus
syndrome; Batten disease; benign recurrent intrahepatic cholestasis; beta-mannosidosis; β-thalassemia;
Bethlem myopathy; Blackfan-Diamond anemia; bleeding disorder (coagulation); blepharophimosis;
Byler disease; C syndrome; CADASIL; calcific aortic stenosis; calcification of joints and arteries;
carbamoyl phosphate synthetase I deficiency; carcinoid syndrome diarrhea; cardiofaciocutaneous
syndrome; cardiovascular disease (CVD); Carney triad; carnitine palmitoyltransferase deficiencies;
cartilage-hair hypoplasia; cblC type of combined methylmalonic aciduria; CD18 deficiency; CD3Z-
associated primary T-cell immunodeficiency; CD40L deficiency; CDAGS syndrome; CDG1A;
CDG1B; CDG1M; CDG2C; CEDNIK syndrome; central core disease; centronuclear myopathy;
cerebral capillary malformation; cerebrooculofacioskeletal syndrome type 4; cerebrooculogacioskeletal
syndrome; cerebrotendinous xanthomatosis; Chagas' Disease; Charcot Marie Tooth Disesase;
chemotherapy; cherubism; CHILD syndrome; chronic granulomatous disease; chronic recurrent
multifocal osteomyelitis; cirrhosis; citrin deficiency; citrullinemia type I; citrullinemia type II; classic
hemochromatosis; CNPPB syndrome; cobalamin C disease; Cockayne syndrome; coenzyme Q10
deficiency; Coffin-Lowry syndrome; Cohen syndrome; combined deficiency of coagulation factors V;
common variable immune deficiency 3; complement hyperactivation; complete androgen insentivity;
cone rod dystrophies; conformational diseases; congenital adrenal hyperplasia (CAH); congenital bile
acid synthesis defect type 1; congenital bile acid synthesis defect type 2; congenital defect in bile acid
synthesis type; congenital erythropoietic porphyria; congenital generalized osteosclerosis; congenital
muscular dystrophy 1A (MDC1A); Cornelia de Lange syndrome; coronary heart disease; Cousin
syndrome; Cowden disease; COX deficiency; Cri du chat syndrome; Crigler-Najjar disease; Crigler-
Najjar syndrome type 1; Crisponi syndrome; Crouzon syndrome; Currarino syndrome; Curth-Macklin
type ichthyosis hystrix; cutaneous T-cell lymphoma; cutis laxa; cystic fibrosis; cystinosis; d-2-
hydroxyglutaric aciduria; DDP syndrome; Dejerine-Sottas disease; dementia; Denys-Drash syndrome;
Dercum disease; desmin cardiomyopathy; desmin myopathy; DGUOK-associated mitochondrial DNA
depletion; diabetes Type I; diabetes Type II; disorders of glutamate metabolism; distal spinal muscular
atrophy type 5; DNA repair diseases; dominant optic atrophy; Doyne honeycomb retinal dystrophy;
Dravet Syndrome; Duchenne muscular dystrophy; dyskeratosis congenita; Ehlers-Danlos syndrome
type 4; Ehlers-Danlos syndromes; Elejalde disease; Ellis-van Creveld disease; Emery-Dreifuss
muscular dystrophies; encephalomyopathic mtDNA depletion syndrome; encephalitis; enzymatic
diseases; EPCAM-associated congenital tufting enteropathy; epidermolysis bullosa with pyloric
atresia; epilepsy; fabry disease; facioscapulohumeral muscular dystrophy; Factor V Leiden
thrombophilia; Faisalabad histiocytosis; familial atypical mycobacteriosis; familial capillary
malformation-arteriovenous; Familial Creutzfeld-Jakob disease; familial esophageal achalasia; familial
glomuvenous malformation; familial hemophagocytic lymphohistiocytosis; familial mediterranean
fever; familial megacalyces; familial schwannomatosis; familial spina bifida; familial splenic
asplenia/hypoplasia; familial thrombotic thrombocytopeniaurpura; Fanconi disease (Fanconi anemia);
Feingold syndrome; FENIB; fibrodysplasia ossificans progressiva; FKTN; Fragile X syndrome;
Francois-Neetens fleck corneal dystrophy; Frasier syndrome; Friedreich's ataxia; FTDP-17; Fuchs
corneal dystrophy; fucosidosis; G6PD deficiency; galactosialidosis; Galloway syndrome; Gardner
syndrome; Gaucher disease; Gitelman syndrome; glaucoma; GLUT1 deficiency; GM2- Gangliosidoses
(e.g., Tay Sachs Disease, Sandhoff Disease) glycogen storage disease type 1b; glycogen storage
disease type 2; glycogen storage disease type 3; glycogen storage disease type 4; glycogen storage
disease type 9a; glycogen storage diseases; GM1-gangliosidosis; Greenberg syndrome; Greig
cephalopolysyndactyly syndrome; hair genetic diseases; hairy cell leukemia; HANAC syndrome;
harlequin type ichtyosis congenita; HDR syndrome; hearing loss; heart failure; hemochromatosis type
3; hemochromatosis type 4; hemolytic anemia; hemolytic uremic syndrome; hemophilia A; hemophilia
B; hepatitis C infection; hereditary angioedema type 3; hereditary angioedemas; hereditary
hemorrhagic telangiectasia; hereditary hypofibrinogenemia; hereditary intraosseous vascular
malformation; hereditary leiomyomatosis and renal cell cancer; hereditary neuralgic amyotrophy;
hereditary orotic aciduria; hereditary sensory and autonomic neuropathy type; Hermansky-Pudlak
disease; HHH syndrome; HHT2; hidrotic ectodermal dysplasia type 1; hidrotic ectodermal dysplasias;
histiocytic sarcoma; HNF4A-associated hyperinsulinism; HNPCC; homozygous familial
hypercholesterolemia; hormone refractory prostate cancer; human immunodeficiency with
microcephaly; Human monkeypox (MPX); human papilloma virus (HPV) infection; Huntington's
disease; hyper-IgD syndrome; hyperinsulinism-hyperammonemia syndrome; hypercholesterolemia;
hypertension; hypertrophy of the retinal pigment epithelium; hypochondrogenesis; hypohidrotic
ectodermal dysplasia; hypotension; ICF syndrome; idiopathic congenital intestinal pseudo-obstruction;
immunodeficiency 13; immunodeficiency 17; immunodeficiency 25; immunodeficiency with hyper-
IgM type 1; immunodeficiency with hyper-IgM type 3; immunodeficiency with hyper-IgM type 4;
immunodeficiency with hyper-IgM type 5; immunoglobulin alpha deficiency; inborn errors of thyroid
metabolism; infantile myofibromatosis; infantile visceral myopathy; infantile X-linked spinal muscular
atrophy; influenza A; influenza B; insulin resistance; intradialytic hypotension; intrahepatic cholestasis
of pregnancy; invasive aspergillosis; invasive mucormycosis; IPEX syndrome; IRAK4 deficiency;
isolated congenital asplenia; Jeune syndrome; Johanson-Blizzard syndrome; Joubert syndrome; JP-
HHT syndrome; juvenile hemochromatosis; juvenile hyalin fibromatosis; juvenile nephronophthisis;
Kabuki mask syndrome; Kallmann syndromes; Kartagener syndrome; KCNJ11-associated
hyperinsulinism; Kearns-Sayre syndrome; Kostmann disease; Kozlowski type of spondylometaphyseal
dysplasia; Krabbe disease; LADD syndrome; late infantile-onset neuronal ceroid lipofuscinosis; LCK
deficiency; LDHCP syndrome; Leber Congenital Amaurosis Teyp 10; Legius syndrome; Leigh
syndrome; lethal congenital contracture syndrome 2; lethal congenital contracture syndromes; lethal
contractural syndrome type 3; lethal neonatal CPT deficiency type 2; lethal osteosclerotic bone
dysplasia; leukocyte adhesion deficiency; Li Fraumeni syndrome; LIG4 syndrome; limb girdle
muscular dystrophies (LGMD1B, LGMD2A, LGMD2B); lipodystrophy; lissencephaly type 1;
lissencephaly type 3; Loeys-Dietz syndrome; low phospholipid-associated cholelithiasis; Lynch
Syndrome; lysinuric protein intolerance; a lysosomal storage disease (e.g., Hunter syndrome, Hurler
syndrome); macular dystrophy; Maffucci syndrome; Majeed syndrome; malaria; mannose-binding
protein deficiency; mantle cell lymphoma; Marfan disease; Marshall syndrome; MASA syndrome;
mastocytosis; MCAD deficiency; McCune-Albright syndrome; MCKD2; Meckel syndrome; MECP2
Duplication Syndrome; Meesmann corneal dystrophy; megacystis-microcolon-intestinal
hypoperistalsis; megaloblastic anemia type 1; MEHMO; MELAS; Melnick-Needles syndrome;
MEN2s; meningitis; Menkes disease; metachromatic leukodystrophies; methymalonic acidemia due to
transcobalamin receptor defect; methylmalonic acidurias; methylvalonic aciduria; microcoria-
congenital nephrosis syndrome; microvillous atrophy; migraine; mitochondrial neurogastrointestinal
encephalomyopathy; monilethrix; monosomy X; mosaic trisomy 9 syndrome; Mowat-Wilson
syndrome; mucolipidosis type 2; mucolipidosis type Ma; mucolipidosis type IV;
mucopolysaccharidoses; mucopolysaccharidosis type 3A; mucopolysaccharidosis type 3C;
mucopolysaccharidosis type 4B; multiminicore disease; multiple acyl-CoA dehydrogenation
deficiency; multiple cutaneous and mucosal venous malformations; multiple endocrine neoplasia type
1; multiple myeloma; multiple sclerosis; multiple sulfatase deficiency; mycosis fungoides; myotonic
dystrophy; NAIC; nail-patella syndrome; nemaline myopathies; neonatal diabetes mellitus; neonatal
surfactant deficiency; nephronophtisis; Netherton disease; neurofibromatoses; neurofibromatosis type
1; Niemann-Pick disease type A; Niemann-Pick disease type B; Niemann-Pick disease type C;
NKX2E; non-alcoholic fatty liver disease (NAFLD); non-alcoholic steatohepatitis (NASH); Noonan
syndrome; North American Indian childhood cirrhosis; NROB1 duplication-associated DSD; ocular
genetic diseases; oculo-auricular syndrome; OLEDAID; oligomeganephronia; oligomeganephronic
renal hypolasia; Ollier disease; Opitz-Kaveggia syndrome; ornithine transcarbamylase deficiency
(OTCD); orofaciodigital syndrome type 1; orofaciodigital syndrome type 2; osseous Paget disease;
osteogenesis imperfecta; otopalatodigital syndrome type 2; orthostatic hypotension; overactive bladder;
OXPHOS diseases; pain; palmoplantar hyperkeratosis; panlobar nephroblastomatosis; Parkes-Weber
syndrome; Parkinson's disease; partial deletion of 21q22.2-q22.3; Pearson syndrome; Pelizaeus-
Merzbacher disease; Pendred syndrome; pentalogy of Cantrell; peroxisomal acyl-CoA-oxidase
deficiency; Peutz-Jeghers syndrome; Pfeiffer syndrome; Pierson syndrome; pigmented nodular
adrenocortical disease; pipecolic acidemia; Pitt-Hopkins syndrome; plasmalogens deficiency; platelet
glycoprotein IV deficiency; pleuropulmonary blastoma and cystic nephroma; pneumonia; polycystic
kidney disease; polycystic ovarian disease; polycystic lipomembranous osteodysplasia; Pompe disease;
including infantile onset Pompe disease (IOPD) and late onset Pompe disease (LOPD); porphyrias;
post-herpetic neuralgia; PRKAG2 cardiac syndrome; premature ovarian failure; primary erythermalgia;
primary hemochromatoses; primary hyperoxaluria; progressive familial intrahepatic cholestasis;
propionic acidemia; prostate cancer; protein-losing enteropathy; pulmonary arterial hypertension;
pyruvate decarboxylase deficiency; RAPADILINO syndrome; renal cystinosis; restless leg syndrome;
retinitis pigmentosa; Rett Syndrome; rhabdoid tumor predisposition syndrome; Rieger syndrome; ring
chromosome 4; Roberts syndrome; Robinow-Sorauf syndrome; Rothmund-Thomson syndrome; severe
combined immunodeficiency disorder (SCID); Saethre-Chotzen syndrome; Sandhoff disease; SC
phocomelia syndrome; SCAS; Schinzel phocomelia syndrome; schizophrenia; severe
hypertriglyceridemia; short rib-polydactyly syndrome type 1; short rib-polydactyly syndrome type 4;
short-rib polydactyly syndrome type 2; short-rib polydactyly syndrome type 3; Shwachman disease;
Shwachman-Diamond disease; sickle cell anemia; Silver-Russell syndrome; Simpson-Golabi-Behmel
syndrome; skin infection; Smith-Lemli-Opitz syndrome; SPG7-associated hereditary spastic
paraplegia; spherocytosis; spinocerebellar ataxia; spinal muscular atrophy; split-hand/foot
malformation with long bone deficiencies; spondylocostal dysostosis; sporadic amyotrophic lateral
sclerosis; sporadic visceral myopathy with inclusion bodies; storage diseases; Stargardt macular
dystrophy; STRA6-associated syndrome; stroke; subependymal glioma; tardive dyskinesia; Tay-Sachs
disease; thanatophoric dysplasia; thromboembolism; thrombosis; thrombophilia due to antithrombin III
deficiency; thyroid metabolism diseases; Tourette syndrome; transcarbamylase deficiency;
transthyretin-associated amyloidosis; trisomy 13; trisomy 22; trisomy 2p syndrome; tuberous sclerosis;
tufting enteropathy; ullrich congenital muscular dystrophy (UCMD); urea cycle diseases; Usher
Syndrome; Van Den Ende-Gupta syndrome; Van der Woude syndrome; variegated mosaic aneuploidy
syndrome; VLCAD deficiency; von Hippel-Lindau disease; von Willebrand disease; Waardenburg
syndrome; WAGR syndrome; Walker-Warburg syndrome; Werner syndrome; Wilson's disease;
Wiskott-Aldrich Syndrome; Wolcott-Rallison syndrome; Wolfram syndrome; X-linked
agammaglobulinemia; X-linked chronic idiopathic intestinal pseudo-obstruction; X-linked cleft palate
with ankyloglossia; X-linked dominant chondrodysplasia punctata; X-linked ectodermal dysplasia; X-
linked Emery-Dreifuss muscular dystrophy; X-linked lissencephaly; X-linked lymphoproliferative
disease; X-linked visceral heterotaxy; xanthinuria type 1; xanthinuria type 2; xeroderma pigmentosum;
XPV; and Zellweger disease.

TABLE 5.1 provides illustrative RNA-associated diseases or disorders for compositions, systems and methods described herein.

TABLE 5.1
Diseases or Disorders
Exemplary Diseases and Syndromes

Acne, Active Pulmonary Tuberculosis, Active Tuberculosis, Acute Lymphoblastic Leukemia, Acute
Myeloid Leukemia, Age-related Macular Degeneration, Alcoholic Liver Disease, Angiogenesis in
Glioma, Aortic aneurysm, Asthma, Ataxia Telangiectasia, Atherosclerosis, Autosomal Dominant
Cerebellar Ataxia, Autosomal Dominant Polycystic Kidney Disease, Autosomal Recessive Limb-girdle
Muscular Dystrophy, Autosomal Recessive Muscular Dystrophy, Basal Cell Carcinoma, Bladder
Cancer, Breast Cancer, Canavan Disease, Cardiac fibrosis, Cardiac Repair after Myocardial Infarction,
Cardiovascular Disease, Carotid plaque rupture, Cataract, Cerebral development, Cervical Cancer,
Cervical Carcinoma, Cholangiocarcinoma, Chromosome 1p36 Deletion Syndrome, Chronic
Lymphocytic Leukemia, Chronic myeloid leukemia, Chronic Thromboembolic Pulmonary
Hypertension, Chronic Thromboembolic Pulmonary Hypertension, Clear cell renal cell carcinoma,
Cleidocranial Dysplasia, Colon Cancer, Colorectal Cancer, Cone-rod Dystrophy, Coronary Artery
Disease, Coronary artery disease, Coronary atherosclerotic heart disease, Crohn's Disease, Cutaneous
Squamous Cell Carcinoma, Diabetes mellitus, Diabetes Retinopathy, Diabetic Cataract, Diabetic
Nephropathy, Diabetic Retinopathy, Digestive System Neoplasm, Dilated cardiomyopathy, Down
Syndrome, Duchene Muscular Dystrophy, Early-Stage Lung Adenocarcinoma, Ebola virus disease,
Endometrial cancer, Epithelial Ovarian Cancer, Epithelial Ovarian Carcinoma, Esophageal Cancer,
Esophageal Squamous Cell Carcinoma, Essential Hypertension, Facet joint Osteoarthritis, Familial
Adenomatous Polyposis, Familial Mediterranean Fever, Familial Temporal Lobe Epilepsy, Fragile X-
associated Tremor, Friedreich Ataxia, Gall bladder Cancer, Gastric Cancer, Gestational Diabetes
Mellitus, Glaucoma-induced retinal neurodegeneration, Glioblastoma, Glioblastoma Multiforme,
Gliomas, Glucocorticoid-induced osteonecrosis of femoral head, Hailey-hailey Disease, Heart disease,
Heart failure, Hematopoiesis, Hepatic carcinoma, Hepatic Steatosis, Hepatoblastoma, Hepatocellular
Carcinoma, Hereditary Hemorrhagic Telangiectasia, Hereditary Neutrophilia, Herpes Simplex Virus 1
Infection, Hirschsprung Disease, Hypertension, Hypopharyngeal Squamous Cell Carcinoma,
Immunosenescence, Immunoglobulin Alpha Deficiency, Infantile Hemangioma, Inflammatory Bowel
Disease, Intervertebral disc degeneration, Intervertebral Disc Degeneration, Intestinal injury and repair,
Intimal Hyperplasia, Intracranial aneurysms, Irradiated Hepatic Stellate Cell, Ischemic Stroke,
Kawasaki Disease, Kennedy Disease, Klinefelter Syndrome, Lafora Disease, Laryngeal Squamous Cell
Cancer, Laryngeal Squamous Cell Carcinoma, Leukoaraiosis, Liver Cancer, Liver Cell Carcinoma,
Lung Adenocarcinoma, Lung Cancer, Lung Squamous Cell Carcinoma, Lupus Nephritis,
Lymphangiogenesis, Lymphoblastic Lymphoma, Macular Degeneration, Machado-Joseph Disease,
Major Depressive Disorder, Malignant Melanoma, Malignant Tumours, Moyamoya Disease, Multiple
Cancers, Multiple Myeloma, Multiple Sclerosis, Multiple system atrophy, Myocardial Infarction,
Myocardial senescence, Nasopharyngeal Carcinoma, Nervous inflammatory diseases or immune
disorders, Non-Small cell Lung Cancer, Oral Squamous Cell Carcinoma, Osteoarthritis, Osteoporosis,
Osteosarcoma, Ovarian Cancer, Ovarian Carcinoma, Ovarian Endometriosis, Pancreatic Cancer,
Pancreatic Ductal Adenocarcinoma, Papillary Thyroid Carcinoma, Patau Syndrome, Pelizaeus-
Merzbacher Disease, PDLSC Osteogenis, Pituitary Adenomas, Polycystic Liver Disease,
Postmenopausal Osteoporosis, Pre- Eclampsia, Pre-diabetes and type 2 Diabetes mellitus, Primary
Biliary Cholangitis, Primary Great Saphenous Vein Varicosities, Primary Hepatic Carcinoma,
Proliferative Vitreo Retinopathy, Prostate Cancer, Pulmonary Tuberculosis, Renal Cancer, Renal Cell
Carcinoma, Repeated implantation failure, Retinoblastoma, Rhabdomyosarcoma, Rheumatic Heart
Disease, Rheumatoid Arthritis, Salivary Adenoid Cystic Carcinoma, Senile Cataract, Sickle Cell
Anemia, Small cell lung cancer, Spinocerebellar Ataxia, Systemic Lupus Erythematous, T2DM with
depression, Temporal Lobe Epilepsy, Thalassemia, Thoracic Aortic Dissection, Thyroid Cancer,
Thyroid Carcinoma, Triple-negative breast cancer, Tuberculosis, Tumorigenesis, Type 2 Diabetes
Mellitus, Ulcerative Colitis, Various Cancer Cell Types, and Velocardiofacial Syndrome.

### EXAMPLES

The following examples are included for illustrative purposes only and are not intended to limit the scope of the invention.

### Example 1. Metagenomic Identification and Analysis of Effector Proteins and CRISPR Arrays

Genes encoding effector proteins were identified by sequence homology and structural analyses of potential CRISPR arrays and cognate proteins. Cognate proteins having a length from about 1,000 to about 1,500 amino acids were identified and are as set forth in TABLE 1. Without being bound by theory, it is contemplated that identified proteins may enact cis and/or trans cleavage on target RNA and, as such, will be useful in the compositions, methods, devices, and/or diagnostics in accordance with the embodiments described herein.

### Example 2. Effector Proteins Cis Cleave RNA In Vitro

An in vitro screen is carried out to identify RNA-targeting cis cleavage nucleases. Specifically, compositions comprising varying concentrations of an isolated effector protein, a guide nucleic acid (e.g., crRNA) and a target nucleic acid are tested in a cis cleavage assay. Briefly, compositions comprising an effector protein (e.g., 1 μl of 1 μM of any one effector protein listed in TABLE 1), a guide nucleic acid (e.g., 1 μl of 5 μM crRNA), various concentrations of target nucleic acid (e.g., 10 ng/μl, 7 ng/μl, and 5 ng/μl of single stranded RNA), and a buffer (e.g., 20 mM Tricine pH9 at 37° C., 15 mM Mg(OAc)2, 1 mM TCEP, and 0.2 mg/mL BSA) are incubated at 37° C. for different time intervals (e.g., 10 minutes, 20 minutes, 30 minutes, and 1 hour). Cis cleavage activity by effector protein is indicated by a detectable signal that is released upon cleavage of the target nucleic acid to which the guide nucleic acid (e.g., a single guide RNA, a crRNA, comprising a spacer sequence) is hybridized. The detectable signal is recorded every minute for the length of the time interval (e.g., 10 minutes, 20 minutes, 30 minutes, 1 hour), quantified, and compared between conditions to determine relative cis cleavage activity among conditions comprising different effector protein compositions. Cis cleavage is confirmed by electrophoresis.

### Example 3. Effector Proteins Trans Cleave RNA In Vitro

An in vitro screen is carried out to identify RNA-targeting trans cleavage nucleases. Specifically, compositions comprising varying concentrations of an isolated effector protein, a guide nucleic acid (e.g., crRNA) and a target nucleic acid are tested in a trans cleavage assay. Briefly, compositions comprising an effector protein (e.g., 1 μl of 1 μM of any one effector protein listed in TABLE 1), a guide nucleic acid (e.g., 1 μl of 5 pM crRNA), various concentrations of target nucleic acid (e.g., 10 ng/μl, 7 ng/μl, and 5 ng/μl of single stranded RNA or DNA), a buffer (e.g., 20 mM Tricine pH9 at 37° C., 15 mM Mg(OAc)2, 1 mM TCEP, and 0.2 mg/mL BSA), and a reporter nucleic acid (e.g., 20 μl of a reporter RNA comprising a fluorescein sequence and a fluorescein quencher sequence) are incubated at 37° C. for different time intervals (e.g., 10 minutes, 20 minutes, 30 minutes, and 1 hour). Following the hybridization of the guide nucleic acid (e.g., a single guide RNA, a crRNA, comprising a spacer sequence), trans cleavage activity is indicated by a detectable signal that is released upon cleavage of the reporter nucleic acid. The fluorescent signal emitted by the reporter nucleic acid is recorded every minute for the length of the time interval (e.g., 10 minutes, 20 minutes, 30 minutes, 1 hour), quantified, and compared between conditions to determine relative trans cleavage activity among conditions comprising different effector protein compositions. Trans cleavage is confirmed by electrophoresis.

### Example 4: Effector Protein Screen for Trans Cleavage Activity with an ssRNA Reporter

An in vitro screen assay is conducted to identify the trans cleavage reporter activity of effector proteins (e.g., any one of the effector proteins listed in TABLE 1). The various compositions that are assayed include an effector protein, a guide nucleic acid (e.g., crRNA), various concentrations of a target nucleic acid (e.g., 10 ng/μl, 7 ng/μl, and 5 ng/μl of single stranded RNA or DNA), a trans cleavage buffer (e.g., 20 mM of 0.5×M Imidazole pH7.5, KC1, MgC12, BSA, Igepal Ca-630, glycerol), and a reporter nucleic acid (e.g., a fluorophore-quencher (FQ) reporter).

Briefly, to generate a ribonucleoprotein (RNP) complex, an amount of the effector protein (e.g., 40 nM) is incubated with an amount of the crRNA (e.g., 40 nM) for a time interval (e.g., 10 minutes, 15 minutes, 30 minutes), at 37° C. in a buffer. For trans cleavage reactions, 5 μL of the RNP complex is incubated with 15 μL of a mixture comprising the target nucleic acid (e.g., 5 nM), the reporter (e.g., 200 nM), and the trans cleavage buffer for 60 minutes at 37° C. Reporter preference can be determined by varying the nucleic acid sequence between the fluorophore and the quencher, with candidate reporters following the 5′ to 3′ pattern of F-TA-X-GC-Q, where F is the fluorophore (56-FAM), Q is the quencher (3IABkFQ), T is thymine, A is adenine, G is guanine, C is cytosine, and X is the RNA component that is varied. Systems are screened for 60 minutes at 37° C. Trans cleavage activity is detected by fluorescence signal produced upon cleavage of the fluorophore-quencher reporter.

### Example 5: Effector Protein Screen for Trans Cleavage Activity with an ssRNA Reporter

An effector protein (e.g., any one of the effector proteins listed in TABLE 1) screening assay is conducted to identify the trans cleavage activity on a reporter (e.g., horseradish peroxidase reporter or HRP reporter) immobilized to a solid plate in vitro. The solid plate is a streptavidin-coated multi-well plate. The reporter comprises a biotin functionality on its 3′ end and an HRP enzyme conjugated to its 5′ end, with a nucleic acid linker therebetween comprising a cleavable RNA-based linker section flanked by two uncleavable DNA-based linker sections: 5′ HRP-TTT TTT TTT TTT rUrUrUrUrU-TTT TTT TTT TTT-3′ Biotin-TEG, where T=thymine and rU=ribouridine (SEQ ID NO: 118). The various compositions that are assayed include an effector protein, a guide nucleic acid (e.g., crRNA), various concentrations of a target nucleic acid (e.g., 10 ng/μl, 7 ng/μl, and 5 ng/μl of single stranded RNA or DNA), a trans cleavage buffer (e.g., 20 mM of 0.5×M Imidazole pH7.5, KC1, MgC12, BSA, Igepal Ca-630, glycerol), and the reporter nucleic acid (e.g., 200 nM).

Briefly, to generate a ribonucleoprotein (RNP) complex, an amount of the effector protein (e.g., 40 nM) is incubated with an amount of the crRNA (e.g., 40 nM) for a time interval (e.g., 10 minutes, 15 minutes, 30 minutes), at 37° C. in a buffer.

Trans cleavage activity on the immobilized reporter is assayed using the following method. First, to generate an immobilized reporter, an amount of reporter (e.g., 25 μL) is incubated, with intermittent shaking, on a streptavidin-coated 96-well plate at 25° C. for 45 minutes. Once the reporter is immobilized to the surface of the coated well, a 25 μL mixture comprising the target nucleic acid (e.g., 5 nM), the trans cleavage buffer, and about 5 μL of the compositions having the RNP complex is added to the well. The mixture is incubated, with intermittent shaking, in the coated well at 37° C. for 60 minutes. Following incubation, the 25 μL mixture is transferred to a clear 96-well plate. About 50 μL of TMB stabilized chromagen is added to each well prior to measuring an absorbance signal produced upon presence of a detection moiety following trans cleavage of the reporter. Trans cleavage activity is indicated by the absorbance signal measured at 650 nm for 15 min at 37° C.

### Example 6: Workflow for Detecting Target Nucleic Acids Using Immobilized Reporters

An in vitro screen is carried out to detect a plurality of target nucleic acids (RNA or DNA) following cleavage of reporter nucleic acids by activated effector protein-guide nucleic acid complexes. Briefly, compositions comprising varying concentrations of an effector protein (e.g., any one effector protein listed in TABLE 1), a guide nucleic acid (e.g., crRNA), a reporter nucleic acid (e.g., a reporter nucleic acid comprising a fluorescein sequence and a fluorescein quencher sequence), and a target nucleic acid to which the guide nucleic acid is at least partially complementary, are introduced into a device or microfluidic device. Specifically, the reporter nucleic acid and/or guide nucleic acid are immobilized to a spot on a glass slide or retained within a chamber of a device or microfluidic device. Each spot on a glass slide or chamber represents one discrete detection location. The target nucleic acid and the effector protein are added onto the glass slide or introduced into the chamber comprising the reporter nucleic acid and/or guide nucleic acid, and incubated at 37° C. for different time intervals (e.g., 10 minutes, 20 minutes, 30 minutes, and 1 hour). Following the hybridization of the guide nucleic acid, trans cleavage activity is indicated by a detectable signal that is released upon cleavage of the reporter nucleic acid by an activated effector protein-guide nucleic acid complex, thereby indicating the discrete detection location at which a reporter nucleic acid is immobilized. The slide or chambers are imaged to identify the changes in the signal at each discrete detection location, thereby detecting the presence, absence, or amount of target nucleic acid in a sample.

### Example 7: Combining Immobilized Reporters with Instruments Thereof

An in vitro screen is carried out to detect a plurality of target nucleic acids (RNA or DNA) following cleavage of immobilized reporter nucleic acids in a surface-based trans cleavage assay using a microfluidic device. Briefly, compositions comprising varying concentrations of a target nucleic acid are amplified in an amplification chamber of the microfluidic device, including reverse transcription if the target nucleic acid is RNA. The amplified target nucleic acid is transported to a detection chamber of the microfluidic device comprising a plurality of immobilized guide nucleic acids (e.g., crRNA). Immobilized reporter nucleic acids are also located on the substrate surface of the detection chamber at discrete locations and are co-localized with the immobilized guide nucleic acids, such that each discrete detection location comprises at least one immobilized reporter nucleic acid and at least one immobilized guide nucleic acid. Following the addition of an effector protein (e.g., any one effector protein listed in TABLE 1) to the detection chamber, the hybridization of the guide nucleic acid and the target nucleic acid activates the effector protein, initiating trans cleavage of the immobilized reporter nucleic acid. Trans cleavage activity is indicated by a detectable signal or a detectable product, thereby indicating the presence, absence, or amount of the target nucleic acid. A microfluidic device can comprise multiple detection chambers, with different chambers having different immobilized guide nucleic acids, allowing for the simultaneous detection of different target nucleic acids.

### Example 8. Effector Protein Engineering

A cognate effector protein having desirable cleavage activity is identified as described in Example 1, and used as the parental effector protein sequence for protein engineering in accordance with the embodiments described herein.

Without being bound by theory, and in consideration of its protein structure, it is contemplated that the cis and/or trans cleavage activity of the identified cognate effector protein may be modified for enhanced cleavage activity of a target nucleic acid (e.g., under high temperature conditions, high salt conditions, high pH conditions, etc.), enhanced nucleic acid binding activity (e.g., enhanced binding of a guide nucleic acid, and/or target nucleic acid, etc.), improved manufacturability (e.g., improved expressibility, solubility, purification, etc.), or the like, or any combination thereof. Briefly, engineered effector proteins as described herein are generated from the parental effector protein sequence, such that the engineered effector protein's nuclease activity is more efficient than that of a non-engineered effector protein.

### Example 9. Effector Protein Screen for Trans Cleavage Activity in Different Buffers

An ex vivo screen assay was conducted to identify the trans cleavage reporter activity of effector proteins (e.g., any one of the effector proteins listed in TABLE 1) in two different trans cleavage buffers (Buffer 1, or Buffer 2). Briefly, for each effector protein assayed (effector protein comprising SEQ ID NO: 5 or 7), a nucleic acid sequence encoding the effector protein was inserted into a plasmid, the plasmid was then transformed into E. coli, and colonies expressing the engineered effector protein were selected for further culture and lysis. Lysates, in which each individual lysate comprised a different effector protein, were diluted and contacted with a crRNA having a repeat sequence of SEQ ID NO: 48. Briefly, to generate a ribonucleoprotein (RNP) complex, an amount of the effector protein (final concentration of effector protein lysate was 0.25%) was incubated with an amount of the crRNA (50 nM) for a time interval (10 minutes, 15 minutes, 30 minutes, or 60 minutes), at 37° C. in a buffer. For trans cleavage reactions, 10 μL of the RNP complex was incubated with 10 μL of a mixture comprising the target nucleic acid (0 nM, or 10 nM of single stranded RNA or DNA), the reporter (200 nM of a fluorophore-quencher (FQ) reporter), and one of the two different trans cleavage buffers for 60 minutes at 37° C. for a total volume of 20 μL (concentrations are listed as final concentration). The first trans cleavage buffer, Buffer 1, comprised 20 mM Imidazole, pH7.5, 50 mM KCl, 5 mM MgCl², 10 ug/ml BSA, 0.01% Igepal Ca-630, and 5% glycerol. The second trans cleavage buffer, Buffer 2, comprised 20 mM HEPES, pH7.5, 2 mM KOAc, 5 mM MgOAc, 1% glycerol, and 0.016% Triton-X 100. Without being bound by theory, Buffer 2 may be more broadly compatible with other reactions (e.g., nucleic acid amplification, signal amplification, etc.) as compared to Buffer 1. The FQ reporter used was a FAM-labeled rU5 reporter: /56-FAM/rUrUrUrUrU/3IABkFQ/ (SEQ ID NO: 100). Systems were screened for 60 minutes at 37° C. Trans cleavage activity was detected by fluorescence signal upon cleavage of the FQ reporter in a DETECTR reaction. FIG. 1 shows exemplary results of the trans cleavage activity of the effector protein assayed (effector protein comprising SEQ ID NO:5 or 7) in the trans-cleavage reaction, each run in two different trans cleavage buffer systems, depicted as area under the curve (au) blanked by subtracting the signal detected in the 0 nM target nucleic acid condition from the signal detected in the 10 nM target nucleic acid condition.

### Example 10. Effector Protein Screen for Trans Cleavage Activity with Different FQ Reporters

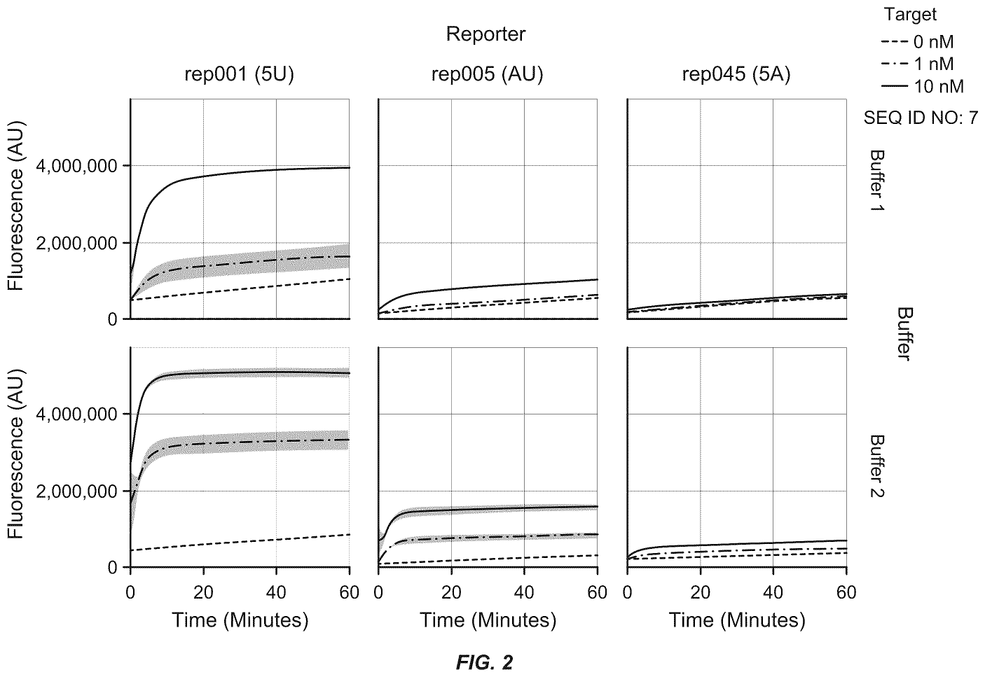

As described in Example 9, an ex vivo screen assay was conducted to identify the trans cleavage reporter activity of effector proteins (e.g., any one of the effector proteins listed in TABLE 1) in two different trans cleavage buffers (Buffer 1, or Buffer 2). Briefly, effector protein (effector protein comprising SEQ ID NO: 7) activity was assayed with three different concentrations of target nucleic acid (0 nM, 1 nM, or 10 nM of single stranded RNA or DNA) and three different FQ reporters (200 nM of rep001, rep005, or rep045). The first FQ reporter, rep001, was a FAM-labeled rU5 reporter: /56-FAM/rUrUrUrUrU/3IABkFQ/ (SEQ ID NO: 100). The second FQ reporter, rep005, was a FAM-labeled rAU reporter: /56-FAMJTArArUGC/3IABkFQ/ (SEQ ID NO: 101). The third FQ reporter, rep045, was a FAM-labeled rA5 reporter: /56-FAM/rArArArArA/3IABkFQ/ (SEQ ID NO: 102). FIG. 2 shows exemplary results of the trans cleavage activity of the effector protein (effector protein comprising SEQ ID NO: 7) on any one of the three different FQ reporters, in the presence of three different concentrations of target nucleic acid, each run in one of the two different trans cleavage buffers. As depicted, the signal detected highlights an apparent preference for rU-rich reporters compared to rA-rich reporters under the conditions tested.

### Example 11. Effector Protein Screen for Trans Cleavage Activity with Different Guide Nucleic Acids

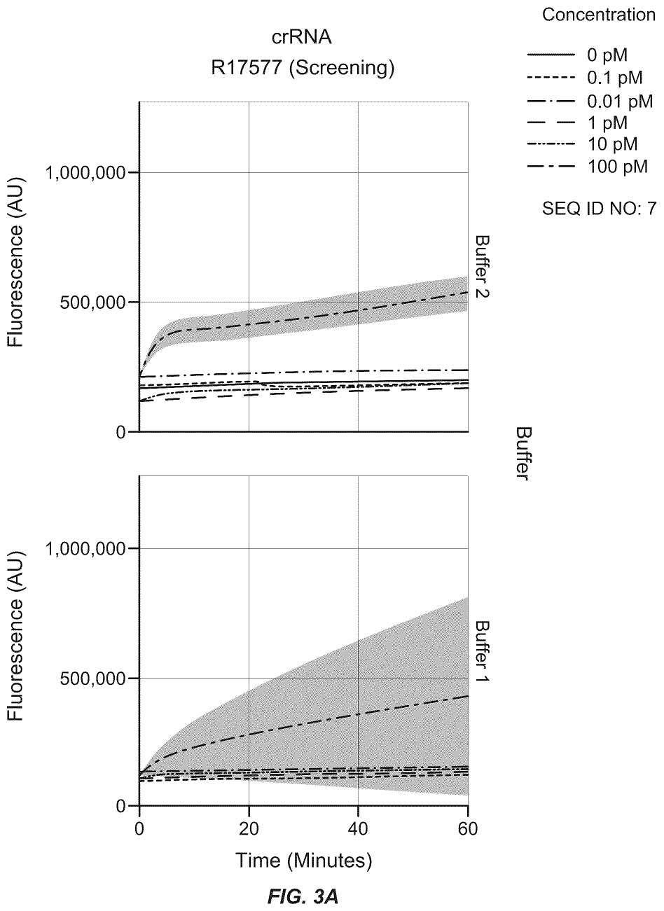

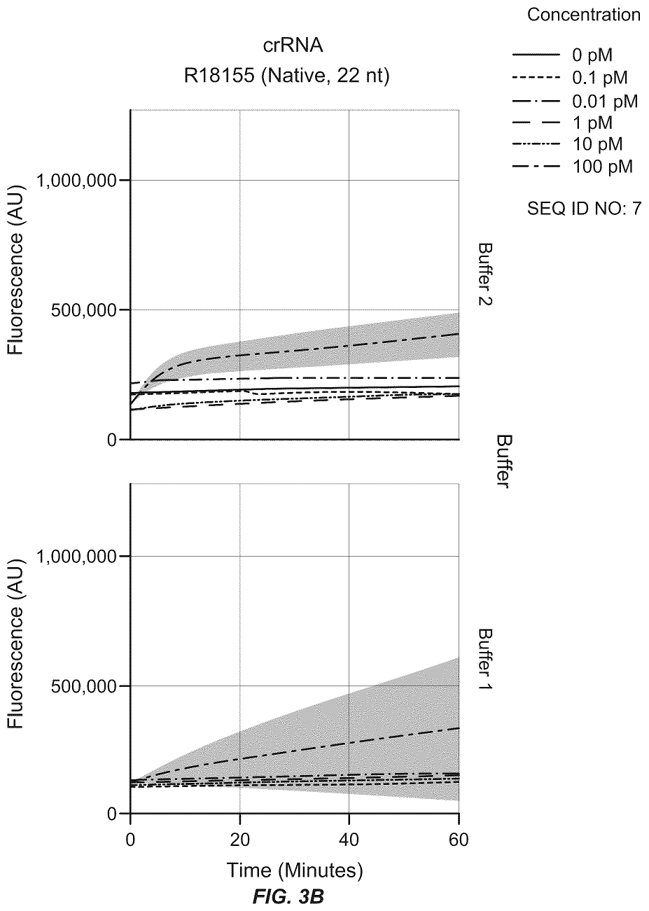

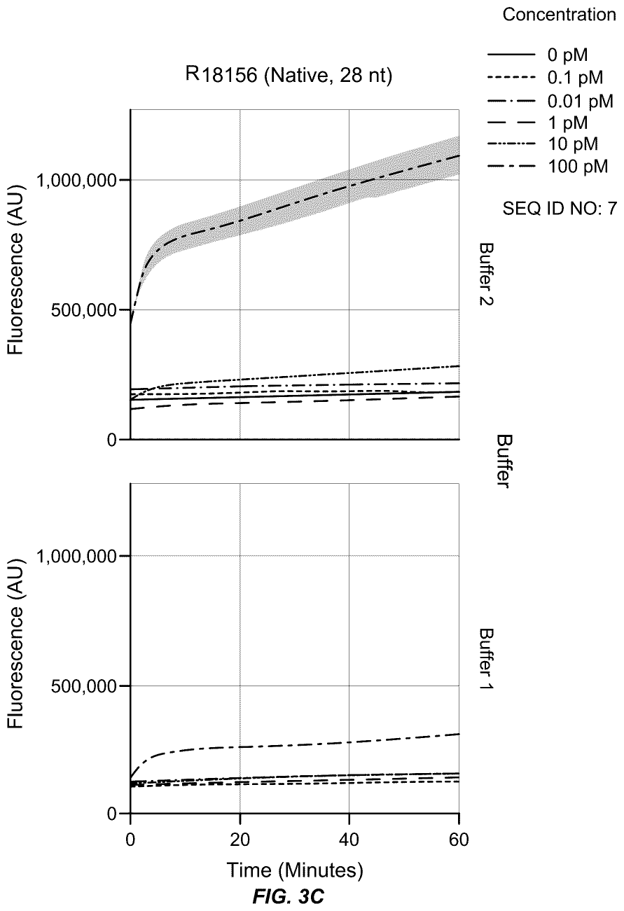

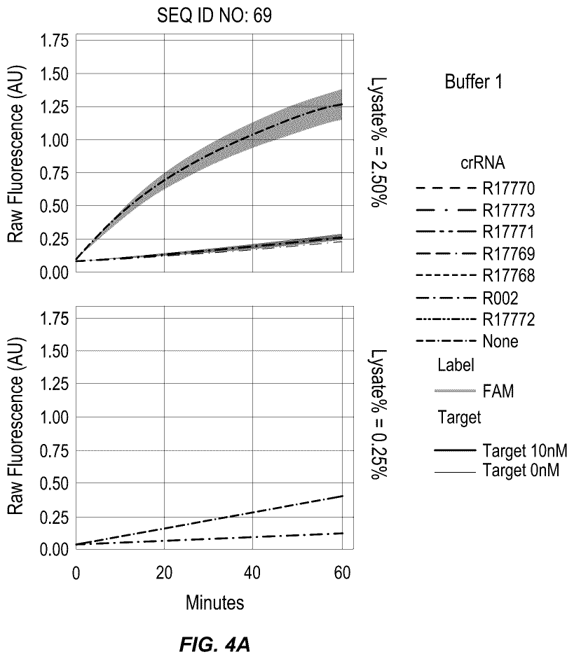

Effector proteins were isolated and purified in accordance with the embodiments described herein, generating purified effector proteins. An in vitro screen assay was conducted to identify the trans cleavage activity of purified effector proteins (e.g., any one of the effector proteins listed in TABLE 1) in two different trans cleavage buffers (Buffer 1, or Buffer 2) as related to three different guide nucleic acids (crRNA having a repeat sequence of SEQ ID NO: 48 and: (i) no spacer sequence (R17577), (ii) a spacer sequence with a length of 22 nucleotides (R18155), or (iii) a spacer sequence with a length of 28 nucleotides (R18156)). Briefly, to generate a ribonucleoprotein (RNP) complex, an amount of the purified effector protein (40 nM, as a final concentration) was incubated with an amount of the crRNA (40 nM, as a final concentration) for a time interval (10 minutes, 15 minutes, 30 minutes, or 60 minutes), at 37° C. in a buffer. For trans cleavage reactions, 10 μL of the RNP complex was incubated with 10 μL of a mixture comprising any one of the six different final target nucleic acid concentrations (0 nM, 0.01 nM, 0.1 nM 1 nM, 10 nM, or 100 nM of single stranded RNA or DNA), the reporter (200 nM of a fluorophore-quencher (FQ) reporter), and one of the two different trans cleavage buffers for 60 minutes at 37° C. for a total volume of 20 μL (concentrations listed as final concentration). The first and second trans cleavage buffer, Buffer 1 and Buffer 2, respectively, were as described in Example 9. The FQ reporter used was a FAM-labeled rU5 reporter: /56-FAM/rUrUrUrUrU/3IABkFQ/ (SEQ ID NO: 100). Systems were screened for 60 minutes at 37° C. Trans cleavage activity was detected by fluorescence signal upon cleavage of the FQ reporter in a DETECTR reaction. FIGS. 3A-3C show exemplary results of the trans cleavage activity of the effector protein (effector protein comprising SEQ ID NO: 7) as related to three different guide nucleic acids and six different target nucleic acid concentrations, each run in one of the two different trans cleavage buffers. Under the conditions tested, trans cleavage activity of the effector protein (effector protein comprising SEQ ID NO: 7) as related to the R18156 guide nucleic acid (crRNA) generated a stronger fluorescence signal, though the overall sensitivity of the system to different target nucleic acid concentrations does not appear to have been affected.

### Example 12. Effector Protein Screen for Trans Cleavage Activity with Different Guide Nucleic Acids in Different Buffer Systems

An ex vivo screen assay was conducted to identify the trans cleavage reporter activity of effector proteins (e.g., any one of the effector proteins listed in TABLE 1) in two different trans cleavage buffers (Buffer 1, or Buffer 2) at two different protein concentrations (final concentration of effector protein lysate was 0.25% or 2.5%). Briefly, for each effector protein assayed (effector protein comprising SEQ ID NO: 69, or a Cas13 variant serving as a positive control comprising SEQ ID NO: 51), a nucleic acid sequence encoding the effector protein was inserted into a plasmid, the plasmid was then transformed into E. coli, and colonies expressing the engineered effector protein were selected for further culture and lysis. Lysates, in which each individual lysate comprised a different effector protein at a different concentration, were diluted and individually contacted with seven different guide nucleic acids (crRNA comprising repeat sequence SEQ ID NO: 59 (R17768), crRNA comprising repeat sequence SEQ ID NO: 60 (R17769), crRNA comprising repeat sequence SEQ ID NO: 53 (R17770), crRNA comprising repeat sequence SEQ ID NO: 55 (R17771), crRNA comprising repeat sequence SEQ ID NO: 56 (R17772), crRNA comprising repeat sequence SEQ ID NO: 54 (R17773), crRNA comprising repeat sequence SEQ ID NO: 57 (R002), or no crRNA. Briefly, to generate a ribonucleoprotein (RNP) complex, an amount of the effector protein (final concentration of effector protein lysate was 0.25% or 2.5%) was incubated with an amount of the guide nucleic acid (40 nM) for a time interval (30 minutes), at 37° C. in a buffer. For trans cleavage reactions, 10 μL of the RNP complex was incubated with 10 μL of a mixture comprising the target nucleic acid (0 nM, or 10 nM of single stranded RNA or DNA), the reporter (200 nM of rep001, (a FAM-labeled rU5 reporter: /56-FAM/rUrUrUrUrU/3IABkFQ/ (SEQ ID NO: 100))), and one of the two different trans cleavage buffers (as described in Example 9) for 60 minutes at 37° C. for a total volume of 20 μL (concentrations listed as final concentration). Systems were screened for 60 minutes at 37° C. Trans cleavage activity was detected by fluorescence signal upon cleavage of the FQ reporter in a DETECTR reaction.

The amino acid sequence of an exemplary positive control (Cas13 variant) used in the experiments included:

(SEQ ID NO: 51)
MKVTKVGGISHKKYTSEGRLVKSESEENRTDERLSALLNMRLDMYIKNPSSTETKENQKRIGKL
KKFFSNKMVYLKDNTLSLKNGKKENIDREYSETDILESDVRDKKNFAVLKKIYLNENVNSEELE
VFRNDIKKKLNKINSLKYSFEKNKANYQKINENNIEKVEGKSKRNIIYDYYRESAKRDAYVSNVK
EAFDKLYKEEDIAKLVLEIENLTKLEKYKIREFYHEIIGRKNDKENFAKIIYEEIQNVNNMKELIEK
VPDMSELKKSQVFYKYYLDKEELNDKNIKYAFCHFVEIEMSQLLKNYVYKRLSNISNDKIKRIFE
YQNLKKLIENKLLNKLDTYVRNCGKYNYYLQDGEIATSDFIARNRQNEAFLRNIIGVSSVAYFSL
RNILETENENDITGRMRGKTVKNNKGEEKYVSGEVDKIYNENKKNEVKENLKMFYSYDFNMDN
KNEIEDFFANIDEAISSIRHGIVHFNLELEGKDIFAFKNIAPSEISKKMFQNEINEKKLKLKIFRQLNS
ANVFRYLEKYKILNYLKRTRFEFVNKNIPFVPSFTKLYSRIDDLKNSLGIYWKTPKTNDDNKTKEI
IDAQIYLLKNIYYGEFLNYFMSNNGNFFEISKEIIELNKNDKRNLKTGFYKLQKFEDIQEKIPKEYL
ANIQSLYMINAGNQDEEEKDTYIDFIQKIFLKGFMTYLANNGRLSLIYIGSDEETNTSLAEKKQEF
DKFLKKYEQNNNIKIPYEINEFLREIKLGNILKYTERLNMFYLILKLLNHKELTNLKGSLEKYQSA
NKEEAFSDQLELINLLNLDNNRVTEDFELEADEIGKFLDFNGNKVKDNKELKKFDTNKIYFDGEN
IIKHRAFYNIKKYGMLNLLEKIADKAGYKISIEELKKYSNKKNEIEKNHKMQENLHRKYARPRKD
EKFTDEDYESYKQAIENIEEYTHLKNKVEFNELNLLQGLLLRILHRLVGYTSIWERDLRFRLKGEF
PENQYIEEIFNFENKKNVKYKGGQIVEKYIKFYKELHQNDEVKINKYSSANIKVLKQEKKDLYIR
NYIAHFNYIPHAEISLLEVLENLRKLLSYDRKLKNAVMKSVVDILKEYGFVATFKIGADKKIGIQT
LESEKIVHLKNLKKKKLMTDRNSEELCKLVKIMFEYKMEEKKSEN.

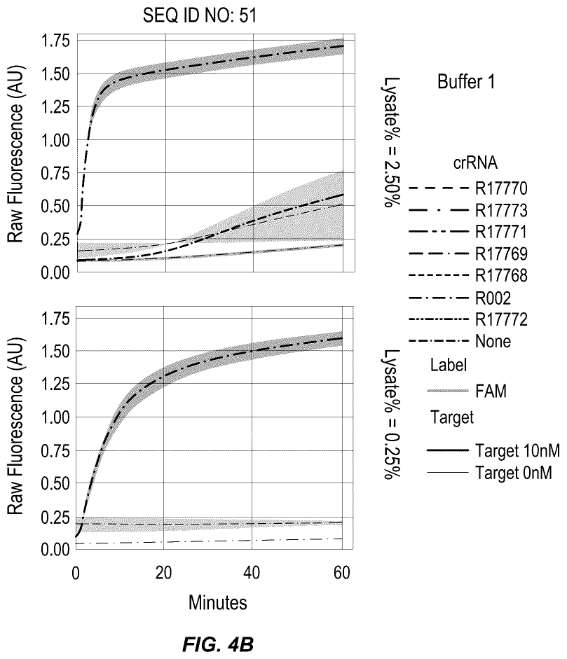

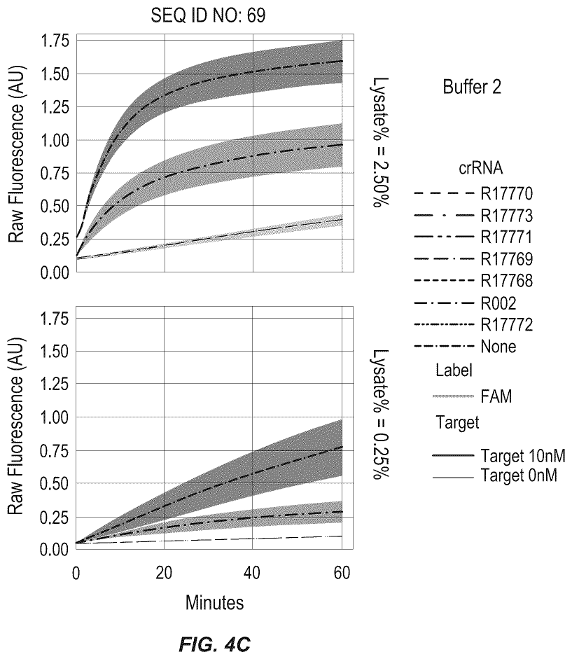

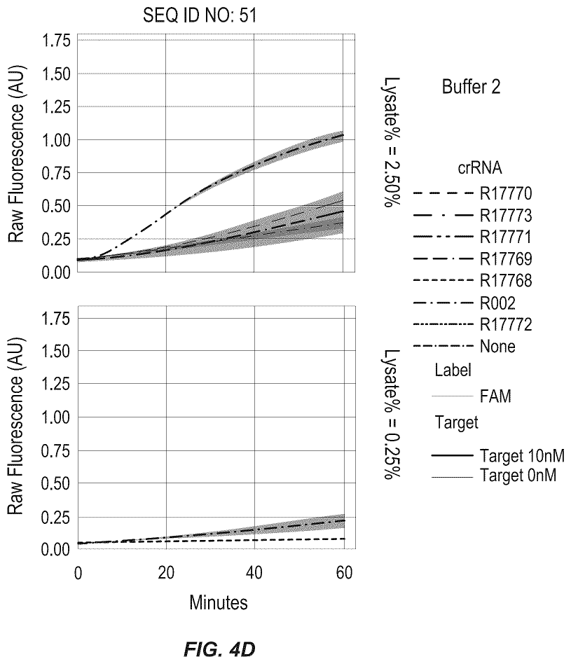

FIGS. 4A-4D show exemplary results of the trans cleavage activity of the effector protein assayed (effector protein comprising SEQ ID NO: 69 as compared to a Cas13 variant serving as a positive control comprising SEQ ID NO: 51) in the trans cleavage reaction, each run with different guide nucleic acids in two different trans cleavage buffer systems, depicted as area under the curve (au) blanked by subtracting the signal detected in the 0 nM target nucleic acid condition from the signal detected in the 10 nM target nucleic acid condition. Exemplary results show the effector protein comprising SEQ ID NO: 69 performed significantly better than the positive control in Buffer 2, as compared to Buffer 1. The effector protein comprising SEQ ID NO: 69 also appeared to have a more flexible spacer preference than the positive control.

### Example 13. Effector Protein Screen for Trans Cleavage Activity with a FQ Reporter in Buffer 2

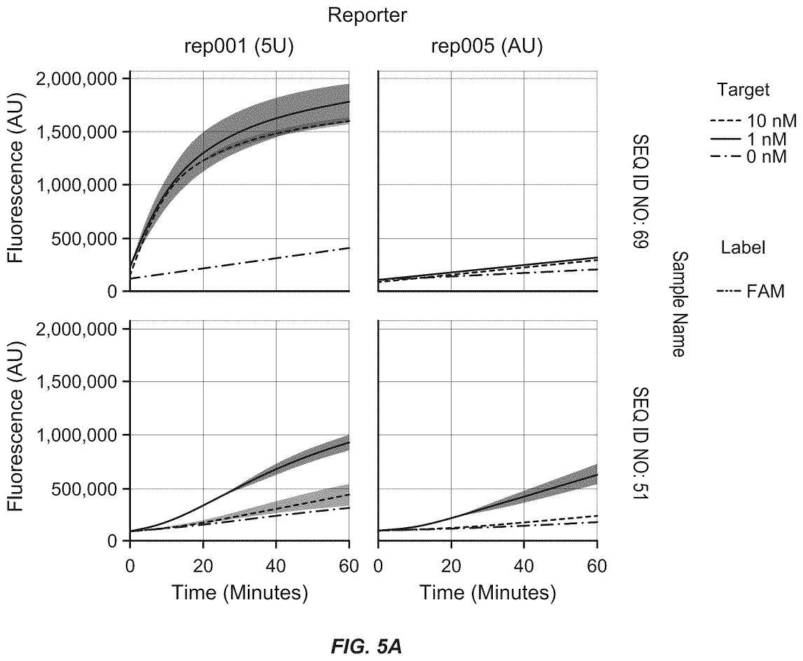

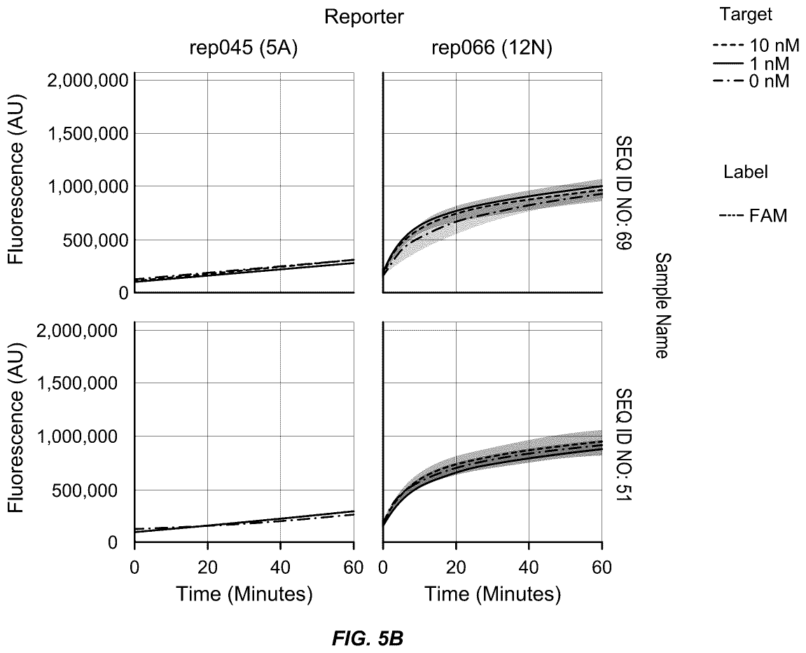

An ex vivo screen assay was conducted to identify the trans cleavage activity of effector proteins (e.g., any one of the effector proteins listed in TABLE 1) mostly as described in Example 12, but in a single buffer (Buffer 2) and at a single protein concentration (final concentration of effector protein lysate was 2.5%). Briefly, effector protein (effector protein comprising SEQ ID NO: 69 as compared to Cas13 variant serving as a positive control comprising SEQ ID NO: 51) activity was assayed with a guide nucleic acid (crRNA comprising repeat sequence SEQ ID NO: 60 (R17769); final concentration of crRNA was 40 nM) three different concentrations of target nucleic acid (0 nM, 1 nM, or 10 nM of single stranded RNA or DNA) and four different FQ reporters (200 nM of rep001, rep005, rep045, or rep066). The fourth FQ reporter, rep066, was a FAM-labeled randomized 12mer reporter: /56-FAM/rNrNrNrNrNrNrNrNrNrNrNrN/3IABkFQ/ (SEQ ID NO: 103). FIGS. 5A-5B shows exemplary results of the trans cleavage activity of the effector protein (effector protein comprising SEQ ID NO: 69 as compared to Cas13 variant serving as a positive control comprising SEQ ID NO: 51) on any one of the four different FQ reporters, in the presence of three different concentrations of target nucleic acid, each run in a trans cleavage buffer. Exemplary results show the signal detected highlights an apparent preference for rU-rich reporters compared to rA-rich reporters under the conditions tested.

### Example 14. Effector Protein Screen for Thermostability of Trans Cleavage Activity

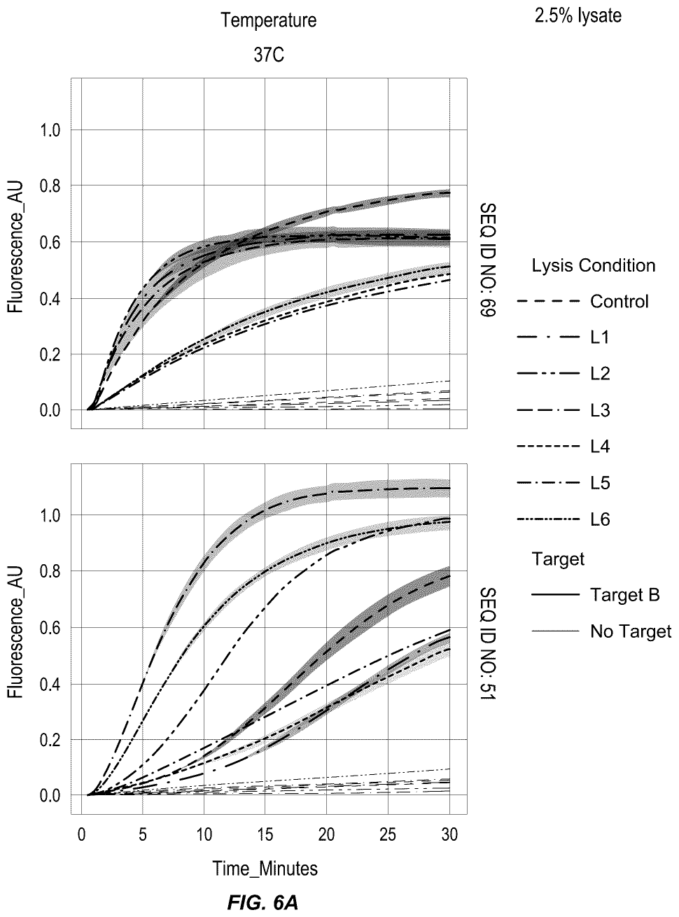

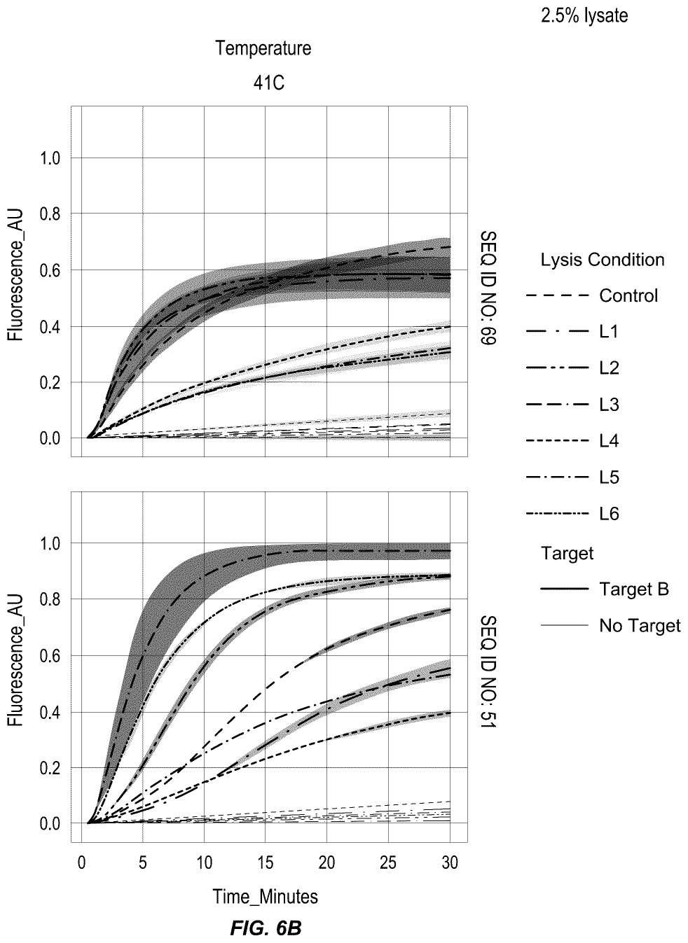

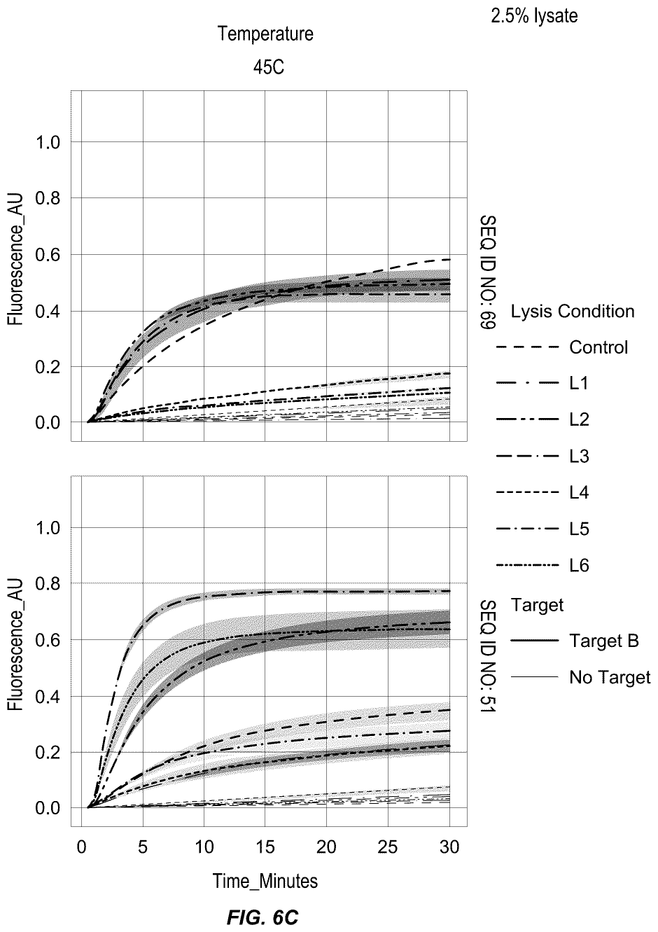

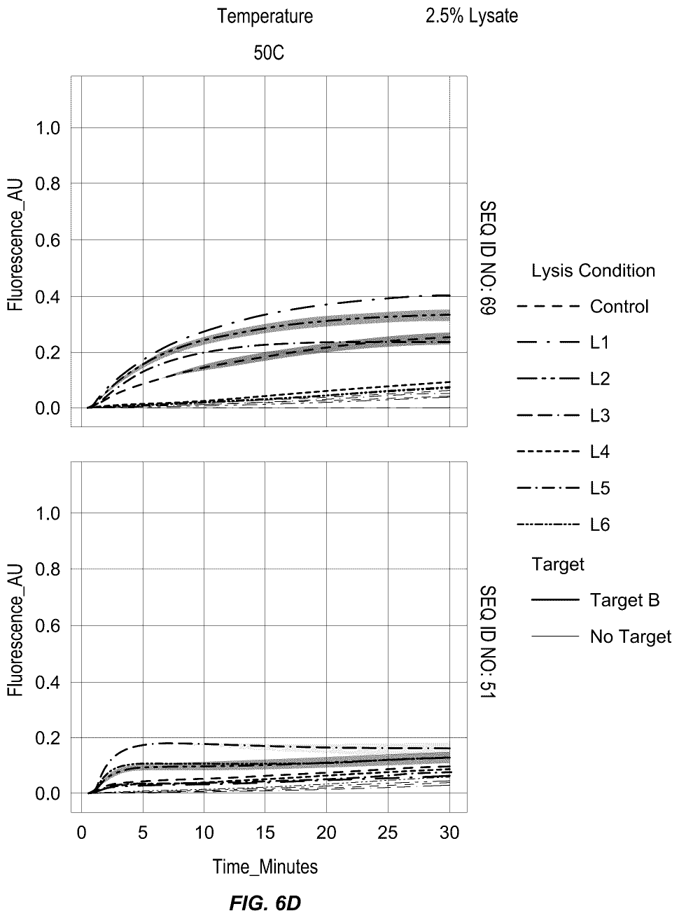

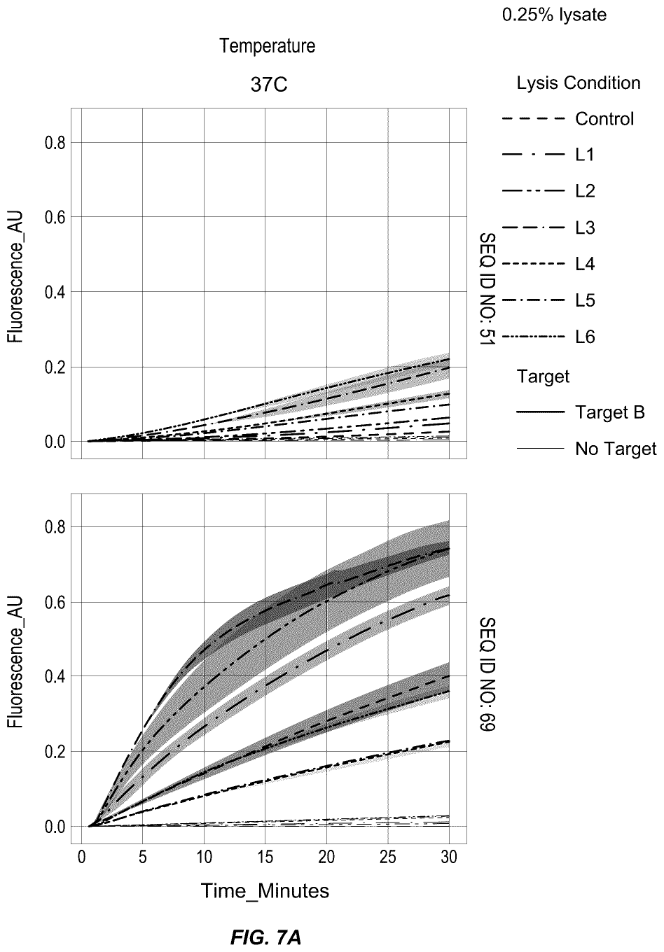

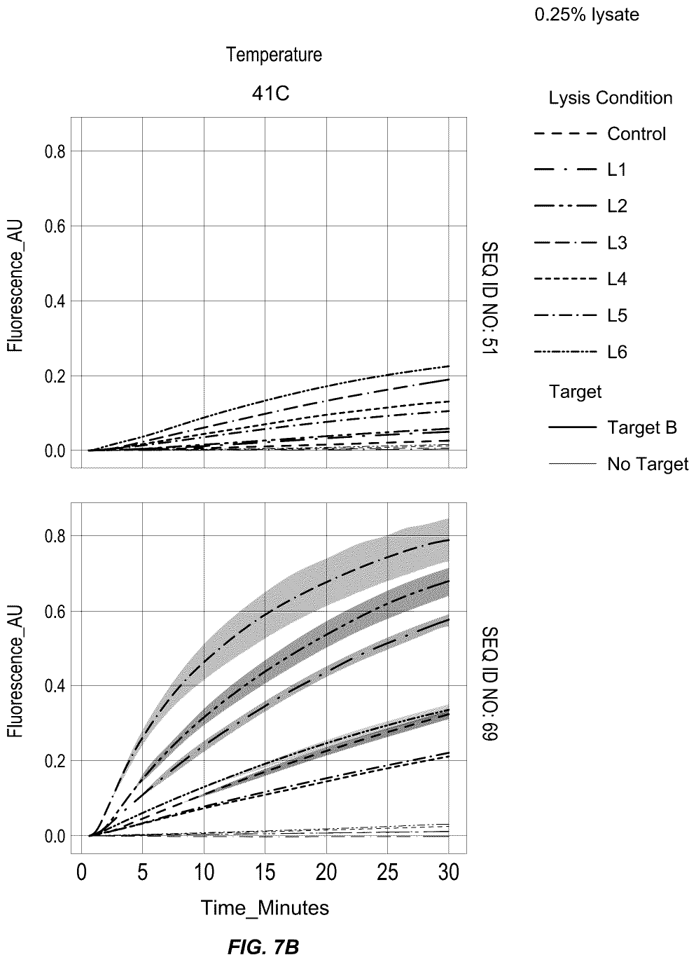

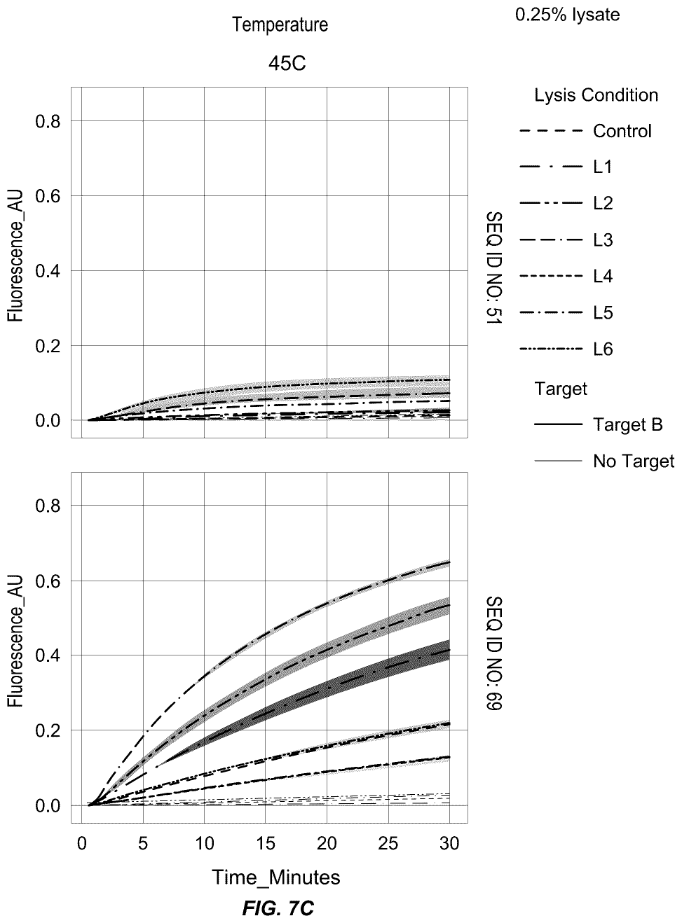

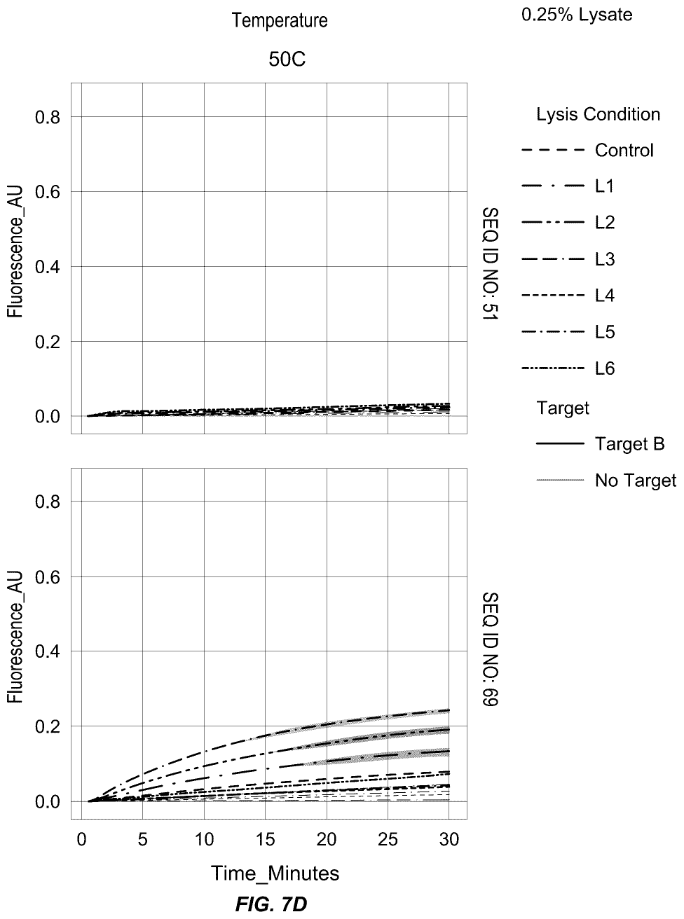

An ex vivo screen assay was conducted to identify the thermostability of the trans cleavage activity of effector proteins (e.g., any one of the effector proteins listed in TABLE 1) in a trans cleavage buffer (Buffer 2), as related to two different effector protein concentrations (final concentration of effector protein lysate was 2.5% or 0.25%) generated with different lysis conditions (L1, L2, L3, LA, L5, or L6, as compared to a positive control). Briefly, for each effector protein assayed (effector protein comprising SEQ ID NO: 69, or a Cas13 variant serving as a positive control comprising SEQ ID NO: 51), a nucleic acid sequence encoding the effector protein was inserted into a plasmid, the plasmid was then transformed into E. coli, and colonies expressing the engineered effector protein were selected for further culture and lysis. Six lysis conditions were tested to determine the effects of varying NaCl concentration and/or TritonX-100 concentration. Lysis conditions Li and LA had a final concentration of 100 mM NaCl. Lysis conditions L2 and L5 had a final concentration of 500 mM NaCl. Lysis conditions L3 and L6 had a final concentration of 1000 mM NaCl. Lysis conditions L1, L2, and L3 had no TritonX-100 while L4, L5, and L6 had 2% TritonX-100. The control lysis condition had 290 nM NaCl and no TritonX-100. Lysates, in which each individual lysate comprised a different effector protein at a different concentration (final concentration of effector protein lysate was 2.5% or 0.25%), were diluted and contacted with a guide nucleic acid (crRNA comprising repeat sequence SEQ ID NO: 60 (R17769); final concentration of crRNA was 40 nM) for a time interval (30 minutes), at 37° C. in a buffer to generate an RNP complex. For trans cleavage reactions, 10 μL of the RNP complex was incubated with 10 μL of a mixture comprising the target nucleic acid (0 nM, or 10 nM of single stranded RNA or DNA), the reporter (200 nM of rep001, (a FAM-labeled rU5 reporter: /56-FAM/rUrUrUrUrU/3IABkFQ/ (SEQ ID NO: 100))), and a trans cleavage buffer (Buffer 2) for a total volume of 20 μL (concentrations listed as final concentration). Systems were screened for 60 minutes at 37° C., 41° C., 45° C., or 50° C. for 60 minutes. Trans cleavage activity was detected by fluorescence signal upon cleavage of the FQ reporter in a DETECTR reaction. FIGS. 6A-6D and FIGS. 7A-7D show exemplary results of the thermostability of the trans cleavage activity of effector proteins (effector protein comprising SEQ ID NO: 69, or a Cas13 variant serving as a positive control comprising SEQ ID NO: 51) in a trans cleavage buffer (Buffer 2), as related to two different effector protein concentrations (final concentration of effector protein lysate was 2.5% or 0.25%) generated with different lysis conditions (L1, L2, L3, LA, L5, or L6, as compared to a positive control).

Exemplary results show the effector protein comprising SEQ ID NO: 69 demonstrated thermostability up to at least 50° C. following most lysis conditions. Additionally, both effector proteins assayed demonstrated improved trans cleavage activity following lysis involving a lysis buffer comprising a higher concentration of NaCl relative to the control concentration of NaCl (290 nM).

### Example 15. Effector Protein Screen for Trans Cleavage Activity with Different Spacer Lengths

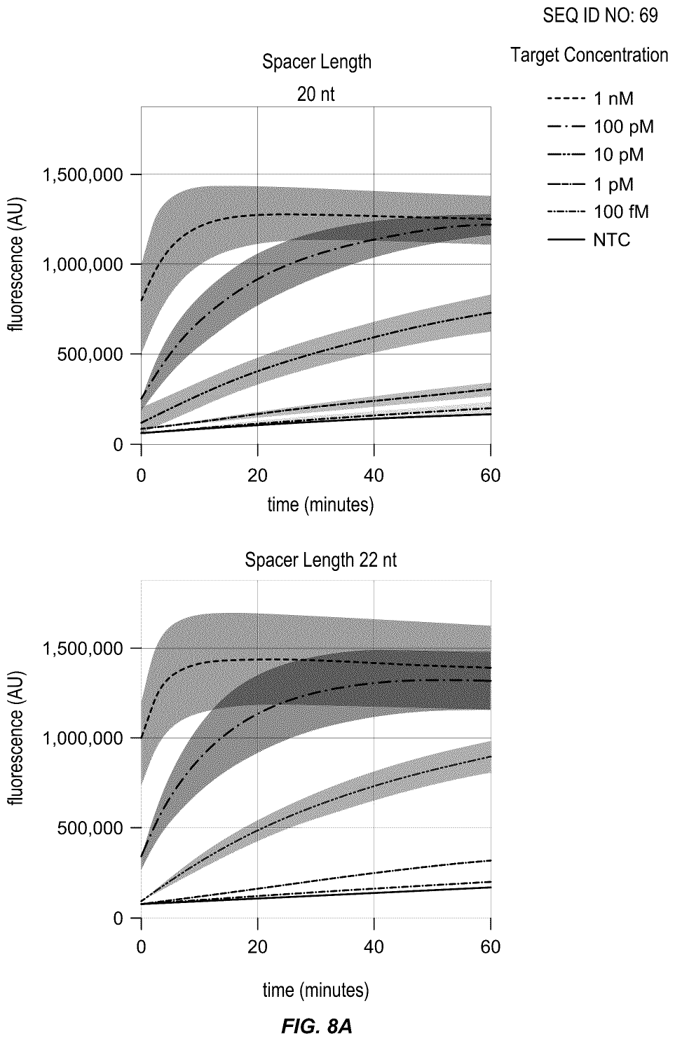

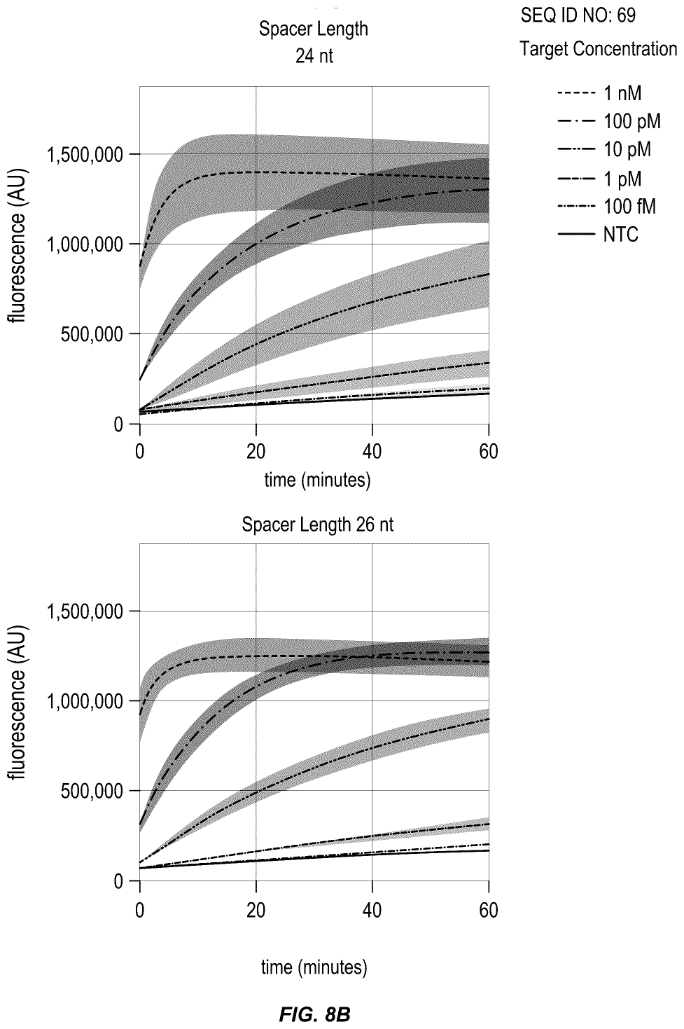

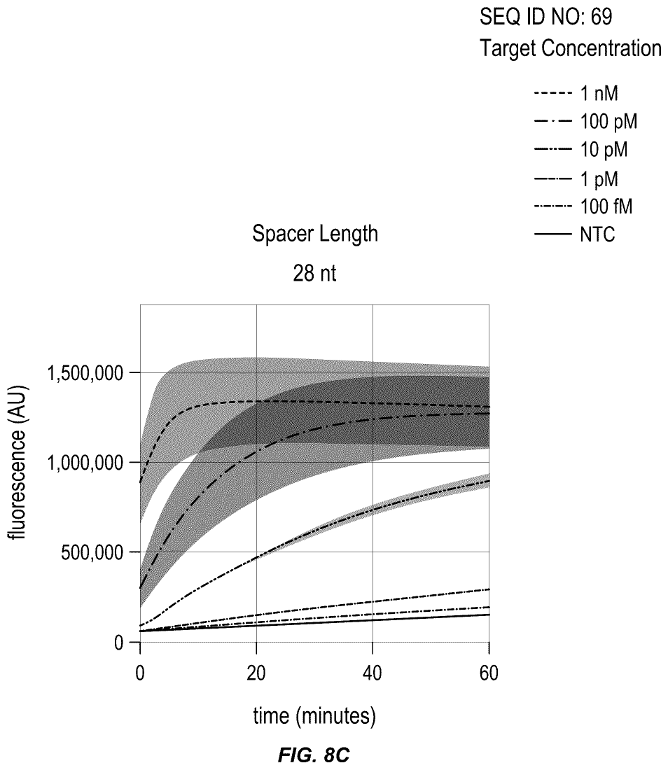

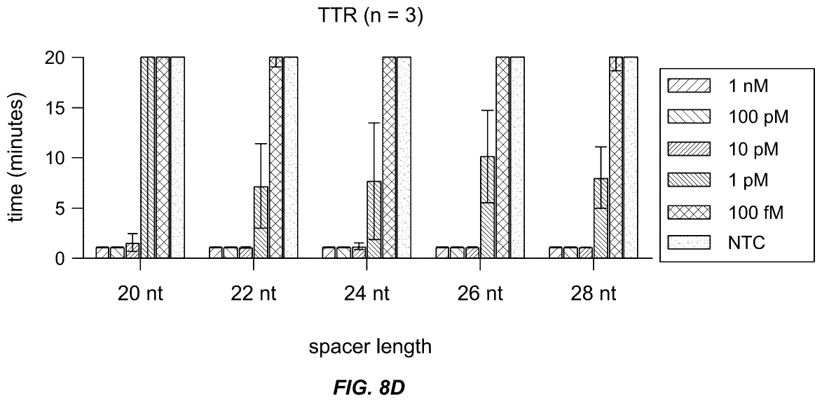

Effector proteins were isolated and purified in accordance with the embodiments described herein, generating purified effector proteins. An in vitro screen assay was conducted to identify the trans cleavage activity of purified effector proteins (e.g., any one of the effector proteins listed in TABLE 1) a trans cleavage buffer (Buffer 2) as related to five different guide nucleic acids (crRNA having a repeat sequence of SEQ ID NO: 283 and: (i) a spacer sequence with a length of 20 nucleotides (R18190), (ii) a spacer sequence with a length of 22 nucleotides (R18692), (iii) a spacer sequence with a length of 24 nucleotides (R18693), (iv) a spacer sequence with a length of 26 nucleotides (R18694), or (v) a spacer sequence with a length of 28 nucleotides (R18695)). Spacer sequences were overlapping such that the first 20 nucleotides were identical and additional target sequence-complementary nucleotides were added to the 3′ end of the 20 nt spacer sequence. Briefly, to generate a ribonucleoprotein (RNP) complex, an amount of the purified effector protein (40 nM, as a final concentration) was incubated with an amount of the crRNA (40 nM, as a final concentration) for a time interval (10 minutes, 15 minutes, 30 minutes, or 60 minutes), at 37° C. in a buffer. For trans cleavage reactions, 10 μL of the RNP complex was incubated with 10 μL of a mixture comprising any one of the six different final target nucleic acid concentrations (0 nM, 100 fM, 1 pM, 10 pM, 100 pM, or 1 nM of single stranded RNA or DNA), the reporter (200 nM of a fluorophore-quencher (FQ) reporter), and the trans cleavage buffer Buffer 2 for 60 minutes at 37° C. for a total volume of 20 μL (concentrations listed as final concentration). The second trans cleavage buffer, Buffer 2, was as described in Example 9. The FQ reporter used was a FAM-labeled rU5 reporter: /56-FAM/rUrUrUrUrU/3IABkFQ/(SEQ ID NO: 100). Systems were screened for 60 minutes at 37° C. Trans cleavage activity was detected by fluorescence signal upon cleavage of the FQ reporter in a DETECTR reaction. FIGS. 8A-8D shows exemplary results of the trans cleavage activity of the effector protein (effector protein comprising SEQ ID NO: 69) as related to five different guide nucleic acids with varying spacer lengths and six different target nucleic acid concentrations. Under the conditions tested, trans cleavage activity of the effector protein (effector protein comprising SEQ ID NO: 69) as related to the 22 nt, 24 nt, 26 nt, and 28 nt spacer sequences generated a stronger fluorescence signal than the 20 nt spacer sequence, though no appreciable difference in either signal strength or time to result was observed with spacer sequences above 22 nt.

### Example 16. Effector Protein Screen for Range of Trans Cleavage Activity with Different Target Concentrations

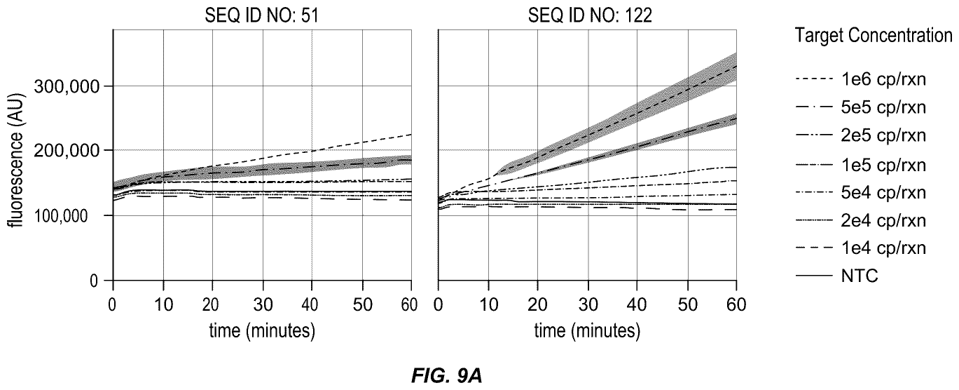

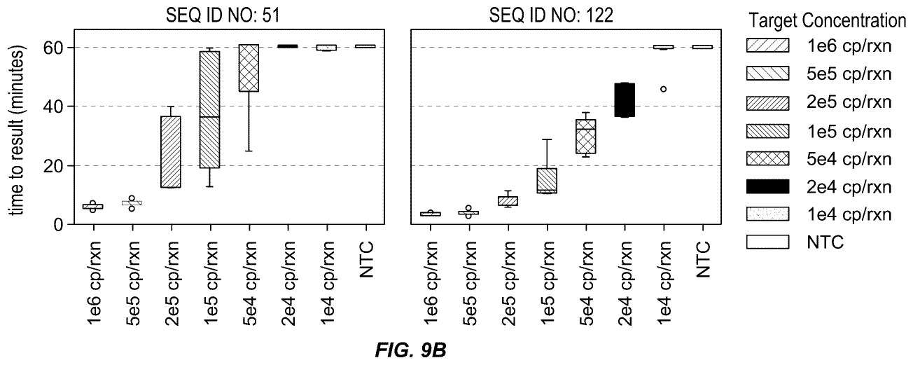

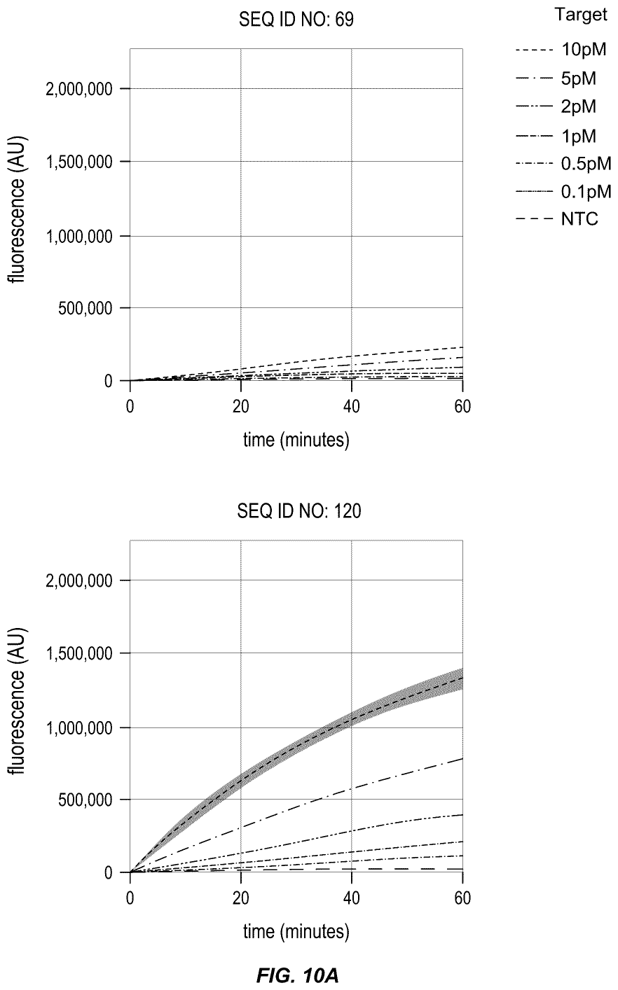

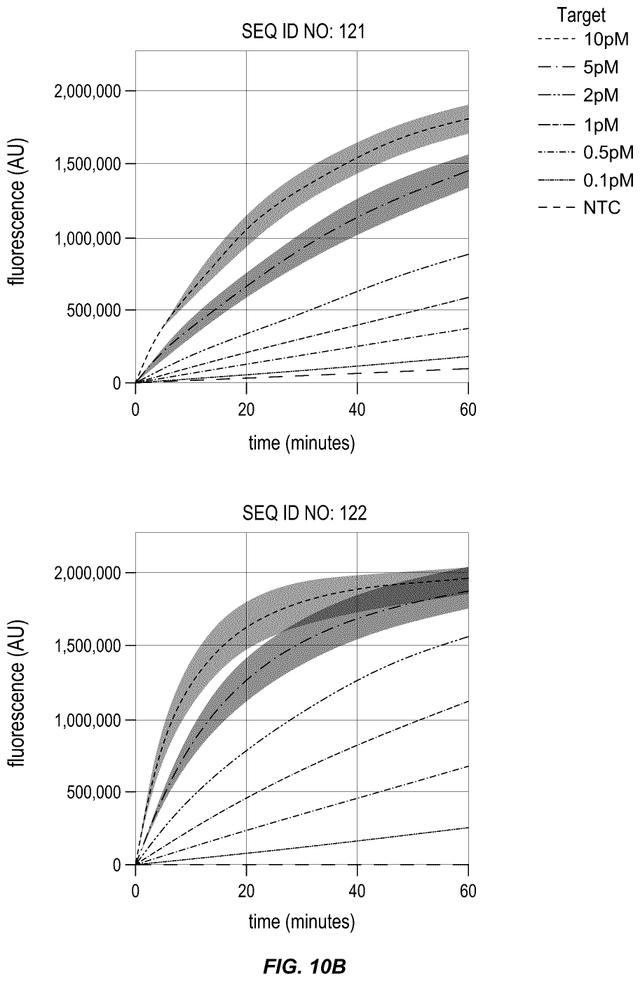

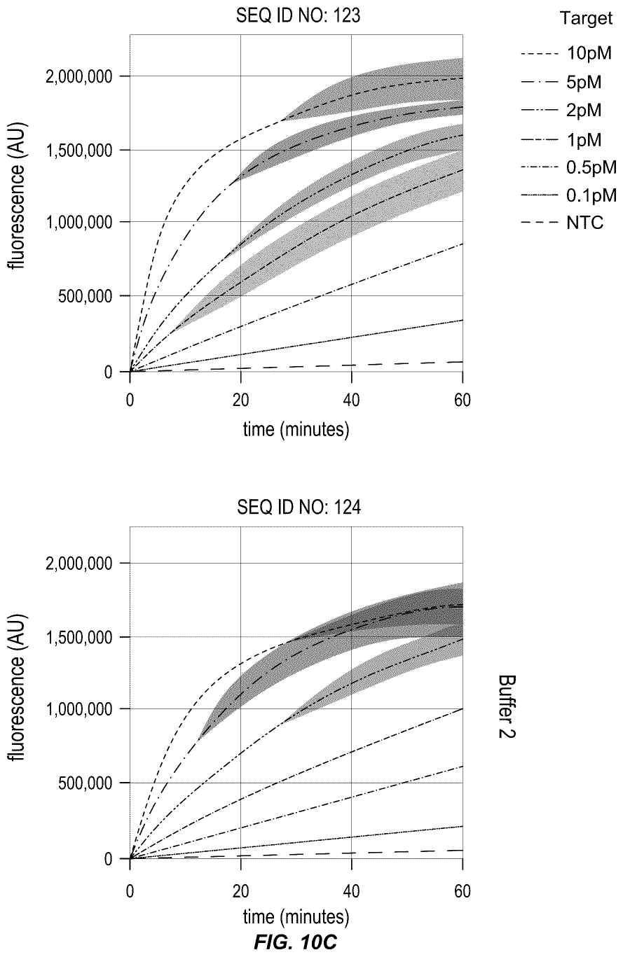

Effector proteins were isolated and purified in accordance with the embodiments described herein, generating purified effector proteins. An in vitro screen assay was conducted to identify the trans cleavage activity of purified effector proteins (e.g., any one of the effector proteins listed in TABLE 1) in a trans cleavage buffer (Buffer 2) as related to eight different target concentrations (0 copies/reaction, 1×10⁴ copies/reaction, 2×10⁴ copies/reaction, 5×10⁴ copies/reaction, 1×10⁵ copies/reaction, 2×10⁵ copies/reaction, 5×10⁵ copies/reaction, or 1×10⁶ copies/reaction) to identify the limit of detection of the system under the conditions tested. Briefly, to generate a ribonucleoprotein (RNP) complex, an amount of the purified effector protein (40 nM, as a final concentration) was incubated with an amount of the crRNA (40 nM, as a final concentration) for a time interval (10 minutes, 15 minutes, 30 minutes, or 60 minutes), at 37° C. in a buffer. For trans cleavage reactions, 10 μL of the RNP complex was incubated with 10 μL of a mixture comprising any one of the eight different final target nucleic acid concentrations (0 copies/reaction, 1×10⁴ copies/reaction, 2×10⁴ copies/reaction, 5×10⁴ copies/reaction, 1×10⁵ copies/reaction, 2×10⁵ copies/reaction, 5×10⁵ copies/reaction, or 1×10⁶ copies/reaction), the reporter (200 nM of a fluorophore-quencher (FQ) reporter), and the trans cleavage buffer Buffer 2 for 60 minutes at 37° C. for a total volume of 20 μL (concentrations listed as final concentration). The second trans cleavage buffer, Buffer 2, was as described in Example 9. The FQ reporter used was a FAM-labeled rU5 reporter: /56-FAM/rUrUrUrUrU/3IABkFQ/(SEQ ID NO: 100). Systems were screened for 60 minutes at 37° C. Trans cleavage activity was detected by fluorescence signal upon cleavage of the FQ reporter in a DETECTR reaction. FIGS. 9A-9B show exemplary results of the trans cleavage activity of each effector protein assayed (effector protein comprising SEQ ID NO: 122, or a Cas13 variant serving as a positive control comprising SEQ ID NO: 51) as related to eight different target nucleic acid concentrations. Under the conditions tested, the effector protein comprising SEQ ID NO: 122 exhibited faster kinetics and a faster time to result, with a limit of detection of about 2×10⁴ copies/reaction, compared to the effector protein comprising SEQ ID NO: 51 which exhibited a limit of detection of about 1×10⁵ copies/reaction in a DETECTR reaction.

### Example 17. Effector Protein Screen for Trans Cleavage Activity with Different Target Concentrations at Elevated Temperature

An in vitro screen assay was conducted to identify the trans cleavage activity of purified effector proteins (e.g., any one of the effector proteins listed in TABLE 1) in a trans cleavage buffer (Buffer 2) as related to eight different target concentrations (0 pM, 0.1 pM, 0.5 pM, 1 pM, 2 pM, 5 pM, or 10 pM) and to two different effector protein concentrations (final concentration of effector protein lysate was 2.5% or 0.25%) generated with different lysis conditions (L1, L2, L3, L4, L5, or L6, as compared to a positive control). Briefly, for each effector protein assayed (effector protein comprising SEQ ID NO: 120, 121, 122, 123, 124, or 69), a nucleic acid sequence encoding the effector protein was inserted into a plasmid, the plasmid was then transformed into E. coli, and colonies expressing the engineered effector protein were selected for further culture and lysis. Six lysis conditions were tested to determine the effects of varying NaCl concentration and/or TritonX-100 concentration. Lysis conditions L1 and LA had a final concentration of 100 mM NaCl. Lysis conditions L2 and L5 had a final concentration of 500 mM NaCl. Lysis conditions L3 and L6 had a final concentration of 1000 mM NaCl. Lysis conditions L1, L2, and L3 had no TritonX-100 while L4, L5, and L6 had 2% TritonX-100. The control lysis condition had 290 nM NaCl and no TritonX-100. Lysates, in which each individual lysate comprised a different effector protein at a different concentration (final concentration of effector protein lysate was 2.5% or 0.25%), were diluted and contacted with a guide nucleic acid (crRNA comprising repeat sequence SEQ ID NO: 60 (R17769); final concentration of crRNA was 40 nM) for a time interval (30 minutes), at 37° C. in a buffer to generate an RNP complex. For trans cleavage reactions, 10 μL of the RNP complex was incubated with 10 μL of a mixture comprising any one of the eight different final target nucleic acid concentrations (0 pM, 0.1 pM, 0.5 pM, 1 pM, 2 pM, 5 pM, or 10 pM), the reporter (200 nM of a fluorophore-quencher (FQ) reporter), and the trans cleavage buffer Buffer 2 for 60 minutes at 45° C. for a total volume of 20 μL (concentrations listed as final concentration). The second trans cleavage buffer, Buffer 2, was as described in Example 9. The FQ reporter used was a FAM-labeled rU5 reporter: /56-FAM/rUrUrUrUrU/3IABkFQ/ (SEQ ID NO: 100). Systems were screened for 60 minutes at 45° C. Trans cleavage activity was detected by fluorescence signal upon cleavage of the FQ reporter in a DETECTR reaction. FIGS. 10A-10C show exemplary results of the trans cleavage activity of each effector protein assayed (effector proteins comprising SEQ ID NO: 120, 121, 122, 123, 124, or 69) as related to eight different target nucleic acid concentrations at 45° C. Under the conditions tested, the effector proteins comprising SEQ ID NO: 120, 121, 122, 123, or 124 exhibited faster kinetics, improved protein expression, improved protein stability, and/or improved sensitivity compared to the effector protein comprising SEQ ID NO: 69.

1. (canceled)

2. (canceled)

3. (canceled)

4. A system comprising a polypeptide, or a recombinant nucleic acid encoding the polypeptide, wherein the polypeptide comprises a variant amino acid sequence of SEQ ID NO: 69, or a functional fragment thereof, wherein the variant amino acid sequence comprises one or more amino acid alterations at one or more residues corresponding to one or more positions listed in TABLE 1.1; and optionally wherein the amino acid sequence, other than the one or more amino acid alterations, has at least 85% sequence identity to the amino acid sequence referenced in SEQ ID NO: 69.

5. The system of claim 4, wherein the one or more amino acid alterations are individually at one or more residues corresponding to one or more positions selected from: 9, 15, 56, 106, 121, 125, 131, 139, 150, 154, 164, 166, 175, 184, 198, 200, 242, 247, 262, 265, 281, 289, 305, 311, 313, 314, 318, 333, 338, 352, 372, 381, 480, 485, 492,496, 501, 517, 521, 537, 543, 546, 547, 548, 555, 559, 567, 569, 574, 579, 585, 618, 621, 622, 623, 631, 647, 656, 684, 705, 709, 717, 722, 726, 737, 747, 762, 765, 766, 769, 789, 790, 800, 801, 807, 819, 827, 836, 843, 846, 847, 857, 858, 864, 867, 870, 871, 909, 915, 919, 923, 927, 974, 1011, 1020, 1030, 1032, 1035, 1049, 1054, 1056, 1062, 1064, 1083, 1085, or combinations thereof, relative to SEQ ID NO: 69.

6. The system of claim 4, wherein the one or more amino acid alterations are individually at one or more residues corresponding to one or more positions selected from: 121, 139, 311, 184, 154, 547, 318, 656, 372, 858, 548, 352, 927, 737, 1062, 819, 501, 974, 1064, 722, 621, 765, 622, 807, 762, 871, 800, 827, 1020, or combinations thereof, relative to SEQ ID NO: 69.

7. The system of claim 4, wherein the one or more amino acid alterations are each a substitution of an amino acid residue with a basic (positively charged) amino acid, an acidic (negatively charged) amino acid, a non-polar (hydrophobic) amino acid, an uncharged polar amino acid, or combinations thereof.

8. The system of claim 4, wherein the one or more amino acid alterations are each a substitution of an amino acid residue with an amino acid residue selected from a group comprising: Gly (G), Lys (K), Ala (A), Gin (Q), Asn (N), Leu (L), Tyr (Y), Arg (R), Glu (E), Met (M), Thr (T), Val (V), Ser (S), His (H), Ile (I), Cys (C), Pro (P), Asp (D), or combinations thereof.

9. The system of claim 4, wherein the one or more amino acid alterations are each a substitution of an amino acid residue with an amino acid residue selected from a group comprising: Asn (N), Gln (Q), Val (V), Glu (E), Lys (K), Leu (L), Ala (A), Cys (C), Ile (I), Ser (S), Pro (P), Thr (T), Tyr (Y), Arg (R), Gly (G), or combinations thereof.

10. The system of claim 4, wherein each of the one or more amino acid alterations are individually selected from a group comprising: T9G, T15K, Q56A, H106Q, E121N, C125L, E131K, H139Q, N150Y, G154K, H164R, Q166K, Q175K, D184E, E198K, F200Y, A242M, R247T, A262T, N265R, N281K, D289T, H305K, M311V, A313S, N314K, K318Q, E333H, L338I, S352C, V372L, L381M, Q480Y, Q485S, V492Q, H496S, V501T, G517N, S521A, L537R, E543L, W546Y, S547N, G548A, I555L, Y559N, N567S, D569H, D574Q, L579V, Q585L, Q618E, W621Q, I622N, I622K, M623L, D631C, L647V, M656L, L684K, A705T, Q709H, K717M, N722R, T726Q, A737S, T747L, A762G, W765R, W765N, Q766M, K769E, T789Q, N790K, D800K, D800G, D800R, E801M, E801G, S807T, S807R, S819K, S827R, S827K, N836P, N843S, A846P, T847K, E857M, Y858L, E864Q, E867A, D870E, N871K, N909D, N909E, E915V, S919Q, S919K, I923L, M927I, D974Y, L1011V, H1020R, A1030S, T1032V, D1035M, D1049G, Q1054T, Q1056L, S1062P, I1064K, P1083Q, A1085E, or combinations thereof, relative to SEQ ID NO: 69.

11. The system of claim 4, wherein each of the one or more amino acid alterations are individually selected from a group comprising: E121N, H139Q, M311V, D184E, G154K, S547N, K318Q, M656L, V372L, Y858L, G548A, S352C, M927I, A737S, S1062P, S819K, V501T, D974Y, I1064K, N722R, W621Q, W765N, I622N, S807R, A762G, N871K, D800R, S827K, H1020R, or combinations thereof, relative to SEQ ID NO: 69.

12. The system of claim 4, wherein the one or more amino acid alterations comprise:
(a) E121N, M311V, S547N, M656L, Y858L, M927I, and S1062P relative to SEQ ID NO: 69;
(b) H139Q, D184E, K318Q, V372L, G548A, and A737S relative to SEQ ID NO: 69;
(c) H139Q, D184E, K318Q, V372L, G548A, A737S, S819K, D974Y, and I1064K relative to SEQ ID NO: 69;
(d) H139Q, D184E, M311V, K318Q, S352C, V372L, V501T, G548A, N722R, A737S, W765N, S807R, S819K, N871K, D974Y, and I1064K relative to SEQ ID NO: 69;
(e) H139Q, G154K, D184E, M311V, K318Q, S352C, V372L, V501T, G548A, I622N, N722R, A737S, W765N, S807R, S819K, S827K, N871K, D974Y, H1020R, and I1064K relative to SEQ ID NO: 69; or
(f) H139Q, D184E, M311V, K318Q, S352C, V372L, V501T, G548A, W621Q, I622N, N722R, A737S, A762G, W765N, D800R, S807R, S819K, N871K, D974Y, and I1064K relative to SEQ ID NO: 69.

13. The system of claim 4, wherein the polypeptide comprises an amino acid sequence that is at least 85% identical to any one of SEQ ID NO: 119-282 listed in TABLE 1.2.

14. The system of claim 4, wherein the system further comprises an engineered guide nucleic acid or a nucleic acid that encodes the engineered guide nucleic acid.

15. (canceled)

16. (canceled)

17. (canceled)

18. (canceled)

19. (canceled)

20. The system of claim 14, wherein the engineered guide nucleic acid comprises a first region or sequence and a second region or sequence, wherein the first region or sequence comprises a repeat sequence, wherein the repeat sequence is at least 75% identical to any one of nucleotide sequences set forth in TABLE 3.

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. The system of claim 20, further comprising a reporter, wherein the reporter comprises a nucleic acid and a detectable moiety, and wherein the nucleic acid comprises RNA, ssDNA, or a combination thereof.

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. A pharmaceutical composition, comprising the system of claim 41.

56. A method of detecting a presence of a target nucleic acid in a sample, the method comprising:
(a) contacting the sample with the system of claim 41;
(b) cleaving a reporter with the polypeptide in response to formation of a complex comprising the polypeptide, an engineered guide nucleic acid, and a target sequence in a target nucleic acid, thereby producing a detectable product; and
(c) detecting the detectable product, thereby detecting the presence of the target nucleic acid in the sample.

57. (canceled)

58. (canceled)

59. (canceled)

60. (canceled)

61. A microfluidic device comprising the system of claim 14 and:
(a) a sample interface configured to receive a sample comprising nucleic acids; and
(b) a chamber fluidically connected to the sample interface;
wherein the chamber comprises the polypeptide and an engineered guide nucleic acid.

62. (canceled)

63. A method for diagnosis comprising the use of the system of claim 14, wherein components of the system further comprises a detectable label or a nucleic acid comprising a detectable label capable of hybridizing to a target nucleic acid.

64. A polypeptide, or a recombinant nucleic acid encoding the polypeptide, wherein the polypeptide comprises a variant amino acid sequence of SEQ ID NO: 69, or a functional fragment thereof, wherein the variant amino acid sequence comprises one or more amino acid alterations at one or more residues corresponding to one or more positions listed in TABLE 1.1; and optionally wherein the amino acid sequence, other than the one or more amino acid alterations, has at least 85% sequence identity to the amino acid sequence referenced in SEQ ID NO: 69.

65. A polypeptide, or a recombinant nucleic acid encoding the polypeptide, wherein the polypeptide comprises an amino acid sequence that is at least 85% identical to any one of SEQ ID NO: 119-282 listed in TABLE 1.2.

66. (canceled)

67. (canceled)

68. The system of claim 4, wherein the polypeptide comprises an amino acid sequence that is 100% identical to SEQ ID NO: 124.


SEQUENCE LISTING (283 sequences)

SEQ ID NO: 1 - protein, 905 aa, synthetic construct
MSHNKKSEIKRIGVKSAFSHGNSRLTITRFGRGNDAEVAVDTDSHGSSLSFPHRVEKSFTVRKIDDEIDVERSSLAASILNPAETVPSDYLGLKSTLEKEFFGEEFPKDSVRIQIIHNILDIQKILGIYINDVLYSIANLQNKEKVEEILGKYLSDKKLNNILPSLRPYMGFLGAGFHSYIKSKSYDDSQNKDADEYNKSVLRILSAIRNDLAHYKKDSLLFSQKLCTGIQDIKKNDWKIINEHYTQMINRINHSFASNSKTNLIILFKIFSTSRSEQQEMVNEYYRFSILKDGKNLGINMRKLRELITDSYFPEIKTTNYDSIRHKIYTILDFVLYKIVRISNELPSIIQELRDSQNTEAKDDLYKKSAWIYGPRLKSQIGNFFEKIRNEISVYTTEELEIEMDSSKFLKDDAEPLVKLLAFLCNFWDGKEVNEILSAYVHKFESIQSFLDTLHDLNDEVVFTSKYDAFNARPENTAYKFAGRAAEQLRVLASIGKMKPDLSGARRALFRDAIEILGIVENSDWLNEDGTFREDMLDQYVLPGNTVSDEEKKKINPFRNFIANNVIESRRFMYLARYSKPKSVRALMRRAEIIKYVLARLPEKQIDTYYTNISDGNDASTFKKIAALTTLLTDFSFTSLYENREAIVENASLSTLNKNVKIERLKALTGLYLTVAFVAVKNLVKANARYFIAFSARERDYAQMLEKDPTLSSLCLFPEGRDPENCYALTEYYLKKEQENDYHPAPGEPFDKEACRKHLDSIHRHFTKKWRDIFRKEIDEALDIHPTGLLGKAVRNQVEHLNILTSALPKYIDDFARNPERTKKWRMTSYFKLYHYLLQRILCDAEELNIPQEWKSRILEKGLPSTDLIHACFVSLGYNLPRYKNLTTEALFDEDSESAKEGKKA

SEQ ID NO: 2 - protein, 1094 aa, synthetic construct
MKGRIASLRDGMSGIVAYGMIKSDIDGSFYFFHKSHLIGCRITEMEKNMAVSFEIRDSQLKPGSKEAYNVALWKDPSAGAIETDKHLEKGIISSFKKNDRNFIFGGMIRSESGSNYFFHRSSVVKGNINAMHEGDKVEFLIGKPEGYDYEQALEVRDLSRSDAAMEERNGEDWRRIERRAQDDEEKREQLLQTIRAEKPETEKRNQSRAKAAGLKSTFTISEHELVMTSFGRGNRAVPEKRIVEGRVEDITAEPVLKVEPRQEQFRIHGRLPEDAYADNPLWISKENPEPQRAGSDLIECRKQLEKMYFGRNFDDNIHIQLIYNILDIDKILSVHINNIVYELNNLRREEKADFDDLIGSMRLGQEYDVFMNPAADSELAREKKEVFLKTMGQKQLSYFGTLLRDDGLRKINDADERARRREAKQKDSYYFLCLLGMARQSLAHGAKKTRAAIYKLDAAFDTENNTARKEARAALDRLYQRKIRSLNANYIERAKADLNILFIAYGVHDDAKKMEIAGEYYDFIVRKEYKNLGFSIRKLRETIEMYHAPELKDTTYDTMRAKLNHIFDFTVFDYYRRNECFGNAMVERLRASLTEAEKEMNYRVEAEELWNEIGARFINSILPRINKDVIKETKDDKHFSVSLEKCDLIGTNATEFSKLIYLFTCFLDGKEINDLLTTMISKFESIEAFIETMDALGLDHTFCADYTLFNESGRIAKELRVINSFARMLDKSDNARPIMYQEAAELLGFPESEDILKRIDEILDKTGSTVKDRKTGFRNFIANNVIESGRFHYLVRYGNVKKLRKLANNRKVIDFVLKDIPDAQILRYYNSVYGKHDPMIRDDMRAALADTITGISFEQFANVHQDDGGTSEEKADKERKKAAVRVYLTVLYLLTKNLVYVNSRYFLAFHCVERDALITDPKRYANKLIRKDFAAFGKTYMPFAKEYVESHNKNWRAQAYLRQDMENSDALMVSLFRNSTEHLNAIRNAERYIDDIAGFSSYFELYHYLMQRTVIDQYERLLGQGKIEEPVNPKMLEYFESVQKHHTYCKDMVKALNVAFAYNLPRYKSLSINGRFDRNRPDVKGKGEKTVDEA

SEQ ID NO: 3 - protein, 963 aa, synthetic construct
MKKKQNAKAIREKLQGQKSEDIRRKEKERQDAIAAERAAEEQRKLELKAQAERDRRARMLRAIKVEKPETEKQNQSRAKAAGLKSTFTISERELVMTSFGRGNRAVPEKRIVEGHVEDIAIEPTLKVEPQQEQFRIHGRLPEDAYAGNPLWIGKENPEPQRNGSDLIGCRKQLEHMYFGRNFEDNIHIQLIYNILDIDKILSVHINNIVYELNNIMRREDADYDDLIGYLDLDTRYEVFMNPDAHYDEKKGRLKTARELKDLFLKMMDQPQVSYFGTVLRDDGLRKIKDRGERAQRMEEKLKDSYYLLCMIGMARQAMAHGDGNTRAAIYKLDRAFDTKNNAARVEARAALDQLYEKKIRALNANFLERAKADLIILFRAFGVNDDATKMKIVGEYYDFIVRKEYKNLGFSIRKLRETIEMYQAPELKDMKYDTLRAKLNHIFDFTVFDYYRRNECIAEAMVERLRASLTEAEKEMNYRIEAERLWKEIGERFMNDVLPRIDGNVIKSIREDKDFSVSLDKCDLIGADATEFSKLIYLLTCFLDGKEINDLLTTLISKFESIEAFLDTMDALGIDHTMAADYTLFNESGRIAKELREINSFARMLDKSDSARQIMYVEAAELLGFPESEDILKRIEEILDKKGSTKWDRKTGFRNFIANNVIESGRFHYLVRYGNAKKLRRLANNRKVIDFVLKDIPDAQILRYYNSVFGKREPMIRDGMRSALAEAITGISFEQFANVHQDKDGTSEQIADKERKKAIVRVYLTVLYLLTKNLVYVNSRYFLAFHCAERDAIVADPDKYLDEKQNVDYYGFAKEFVENHGKNRRAQRYLQVNFANSDEQTVRFFRNCTAHLNAIRNADNYIDDIADFNSYFELYHYLMQRTVIDQYERLRGMGKIKTPVNPKMIGYFELVSKYHTYCKDMVKALNVGFAYNLPRYKNLSIDALFDRNHPGEKGTGKTPVVEE

SEQ ID NO: 4 - protein, 1004 aa, synthetic construct
MKGKVSKKAQREAARNSIISKYSAIDQEREKQRKAEEAIRRENEAARLKEAEVAWRLQREPMRYSRQETGAQNQVEELLRAAALKDRLASEVEKSDKKPSRAKAAGLKSTLVISDTELLMTSFGKGNTAIAEKRITEAGIEAIPEVPAFEVQAQDERFRISGRMVHDAQTDNPLSADKRPGQDLIGCRDQLERLFFGANYEDNVHVQIAYQILDVDKILAIHINNIIYEINNLTRQEGAIDLFEKLGFDTTYDKFSTKTNETIETFNKLLRAPQLAYFGDILYDADLIKRLKKERNEQRKARLLAQEKAKQREAYDLLSMLGQVRHSMAHFNPMIYAMDQQFDAQYTKKGDHFDPAQRLASRAALGQLYAGRVNELNAHFLDRAKVDLQILFAVEKAVLPEDKKRIVRNYYDFIVRKDYKNQGFSIKLLREMMLTFPEASGLLGKTYDSMRARLYRSFDFVVFEHYRQHEQEIDSLVAQLRACCDEKDKQAIYRTEAARVWPLVAPTIMNELLPWMDGQKLAVASSSAMDHTITYDMIKDIAIKPDAACFTKIIYLMTRFLDGKEINDLLTTLINKFENIAGFLAILQEKKIPCSFAQGYDMFGRSAIIAQELRAVNNFARMSEESPEAKRILFVEAAKVLGYPGDDETLGKYIDDMLTAKGEFVARGKGFRNFIANNVIESDRFKYLVRYGNVSQVRRLADNERVLAFVLKDIPDTQIVRYYNSCTGSSRSNCTPDMRQGLAKLMTGISFERFAEVKQSNRATPAEKDDKVRKQAIIRLYLTVLYLLVKNLVYVNSRYFLAFHCVERDSIIYDPERFTPKNIREHYCDFAIERMEGGAWNKRVTQYLRQNMANANSWATRAFRNNVEHLNTVRLASSYIADAGAFASYFELYHYLMQRRLMEEYASDKAEIEARVANGEPREKYAWFPMENEKILTYFGLVKRYHTYCKDMVKALNVPFAYNLPRYKNLSINELFDRNHYLEAKGRGTALATAEAAAQDAGNQ

SEQ ID NO: 5 - protein, 896 aa, synthetic construct
MAESKKTNAKRMGVKAVLAHGTDKLTMTTFGKGNRAEIAFSEGNKGQGLQTPFPIGKKRFSVQNIDEEIYSLGKSDGKLLETRMPNPAGNVGEDYLKFKDILEKQFFGKSFPNDNIRIQIIYLILDIQKILGMNVFDILYTLGNLQDEDIDQFGQSLATDSAVSKVVKTMRPYFGLFGGVFQIPPKDKPEQGMEHNINILRCISSLRNATAHNQEKEFPWFKKADYSKIFRNGKGEWDIIESLYRKKIRRVNDNFAKNSKVNLDILFALLHASEAQKPEITKDYYHFSILKDGKNLGINLVKVRENIIDTFFRQLRNKRYDSQRQKINVIADFLIFRALNSDREFVENAVSRLRLTEDEKAKEKLYRDFARDVWGRVKTNLEPYFRDPKAYILKYKSNGKDYVPEKWFANSKLPETGGNSFVKVLSFLCNFLEGKEINEFLTAYIHKFECIQSFIDTIHELNEEIVFTDRYALFNTKDFAKNVATELRVLASIGKMKPDLEEAKRLLYKYAIQTLGFPVGMEKYVSDEWLEENVLLNAEDRKNRAKKAKVNPFRNFIAGNVIESRRFMYLVRYSKPSTVRALMQNQKIVNYVLHRLPPDQVKSYAQVFPQKFHETEATILYLTEQLAQFSFETLIREKQAILDNSGQRRASESIERLKGITGLYLTVAYIAIKNIVKANARYYIAFSIFERDLRLLTEKAKIEKVTLEPPIVINEKKTIPNPFTLTQYYLDRDNEIKFPGDPRNKEAFFKFLRENKKHFSRQWREWLKEKIMEAKAVQNTGMLLTEARNQVEHLNVLRWMSKYISDFRQGTQPMSSYFELFHYLIQRLLLENPDLNLAPYRDWILRSGRPDRELIQYTFVSLAYNLARYRNLTKEHHFDEVVLESLREEKQENPER

SEQ ID NO: 6 - protein, 953 aa, synthetic construct
MAKKMNPKKLREAEKQRKANQFKVEEEKRKKEREEEEARRVIEEEQRRKAARIASIEKKDPELAKVKKSKVKAAGLKSTFILGDDRLFMTSFGNGAEAKPEKYVIGDTVSDAASKTALNVLSDKNEFHVSGRIIEGARATNPLCTENGKVSYRDDQIHLRSKLEKMYFGHECEDNIHIQLIYQILDIEKILAVSANNIVFTVDNMLRRSDSEHDDLIGYMMGQNYNAYKNANQANKYQLFRELLDKPQLAYFGKTLLPLDVQGKKIKKDTPLWEEYEKRCYYILAALGMVRQVTAHGAEKTRSSMYQLENLSSNSSLYYREMAEVLDQLYSDRVNELNKGFLQKAYKDICIINKILGITDPEKQAETVREYYSFIVLKDYKYLGFSIKKLREYITKNHAEQIAKREYDSVRQKLNHILDFFLYRYYDAPENENILDNLVDKLRSCTKDSEKALIYEDEAAGIWEVMGDMIETRLLPKMDGDYIGSMSEGAADDSLLADIYVKSKGLRFSEMMYVLTIFLDGKEINDLLTQLINRFDNIASFIDVMKHEGINSTFTSKYSIFEKSREISKELRVINSFARMTESSPNVKPIMVIEAAKILGYEDDEEQLQAYVQEMMDKEKGPKNAYGKKDNNFRNFIINNVIESSRFKYLVRYGNPNKLRALASNKKVVDFVLKGIPDEQIMAYYNSCNGVEKAFFPEMRDNLAEKITGLSFMEFSNVYQGKTQDQRLIVDKERKKNIVRIYLTVLYLLLKNLVYVNSRYYMAFHCTERDRGIYDKDKYSSSANERNWSLFAKDYLEAYDWETIKNRRYKQITMNFANADSWSIIAFRNCAEHLTVIRNADEFIEGIAGFESYFELYHYLVQRHLISQFEFECNTDSTRYPGTKIMSTERAEGKLLDYFAKVQKYHTYCKDFVKALCTPFAYNLARYKNLAINELFDRNNYLPSKGKTVEPEE

SEQ ID NO: 7 - protein, 990 aa, synthetic construct
MAKKKVSLREQREAKKNASLAGYRQKSQEQEQKKKEEAAKIEAQKKAAMGPQTAGSTQDADMVLRQRRAKEEKEQRAREGVKRHYAKAAGVKSVYHIRKGELLMTAFGKNAQALPEKKVAGTQIETLADPSAYSAQINEKAFDVVGRLGISARVDNPAYSSKAPGENVVGLKREFEELIFGSHYEDNIHIQIAHNIFKIEKTLSVHINNIVYELDNMLRREDDEHDDLIGYLGLGKKWQTFRNPMPGKEDTYKLFCELLKKPQLSFFGNILYDEELLKQAQKARKAYEKEKAEKKLEQKRENAYDLLCLLGATRQALAHGEASTRSVLYTLDAAYDDEAGKLNGSWVNKNQRLASRNALDRLYADQVRRLNKDFLEHSKVDLQILFDLYGCKDAAEKREIVRAYYDFVVRKEFKNQGFSFTRLRENLLLQKGASVLKEQKYDTMRGRLYRLFDFVLFRYYELHAEESAKLVDKLRATLDETQKEQIYEQEAQRVWAMVKKSVIDHLAQNVSVEYITGHQNADPDVNVQMLAGVALAEHANAFSELMYLLTCFQDGKEINDLLTSLINRFDNIAGQCALLRERGLECTFAPTYRMFAESEKISRELRAINSFARMDMHVEPALKREMFIEAARVLGYPGSDEELLAYADSMLSKDGELVKTTGRKDFRNFIMSNVIASDRFQYIARYCNVNKIRALAANRKVVDFVLKHIPDTQIIRYYNSCEGTGFVELDPRMRDYLAHRITGLSFSDFETVQINERFITSDEKAEKAKKQAIISLYLTVLYLCVKNLVYVNSRYTIAFHFLEHDSITQDKDYRKSLESKGDLRDYAAARIPVKYADKPEKIAALQFIPEERRVPTRETRQREYLLQNLANSDKWAVRTFRNCVQHLNAIRNADSYIGEIDRFDSWFELYHYLVQRSLMDQYRWDSTQKKIISDSEMTGKTLEYFDEIRTYHTYSKDFVKALCVPFGYNLSRFKNLSINELFDKNNYPLK

SEQ ID NO: 8 - protein, 1137 aa, synthetic construct
MQTNYLGKRIEYNYLKTADKHYFGGFLNLAKNNIEVVFSLFCERFKITPTNDISLINDQLKDDVAPSEYQKKVDFLKQYLPVIHYLDLEITNEKFSKEKDKEKAKRKYFRENFIALIKAIGCLRDFYTHHYHKPIFFDVNLFDLLDELLITVVFDVRKRKMKGDQTRHLLKKGLHSEIEELVKLKTIHLKEQKKEGKRVNLDSEAIENAVFNDAFYHLLYKEGVSRNYQSKLISETENENNLNISESGLLFLLSIFLQRKENEQLRANIKGYKGKGKDNEKPIDRNNNSLKFMATHWVFNYLSVKTIKHRLNTTFQKETLLIQIADELSKVPDALYQTFTKEQKEEFVEDVNEYLKDGDSSESLEKSIIIHPVIRKRYENKFNYFSLRFLDEFVNFPSLRFQIYLGNYVQDRRVKTISGTNYTTERMIKEKINVFGKLSEISQIKSDYFGSSNEDTHWEMFPNPSYNFVGNNIPIYINLEKSNVEGAGKLNGLLNQLHKNQEIKNRKENKPSKTEMISELHEGIIFGKPTAVLSMNEMQALLHEVLVNNKTGEEVEKILINKLIERYDTIINFDPNEPLPTSKITKKLRKSTNEIKIDTDKLLRAIESEIRVTNEKLCLIKNNVQELNDRQQKRKFVFTNKELGQEATWLADDIKRFMPKETKENWKGYQHSLLQELLAFYESKPKEAHNFLNTHWNIKDNNYPFNEWLVKAFQNNKFDRFYRVYLSNRVKYFESLKTQISGFKNNRKLLNKFLKQQFVWSIFYKRLYEISTTENQKEQLLLKPLVFPRGIFDEKPTYIKGENIVESPNLFADWYRFIQDKNQELQKFYLWERDYKELFEQHKIALEFSTNKYGLSEIDKFNLFKRKWDNKIKATIGQDLFLNLMNRKIIKDLYNHDVDLSLSDYYLTQKERIEKQNKALKQSQREKQDKSENIVKDDFIWSMTVSFKTEFIDEPSIKLKDVGKFTRFLNEEKVKRIFEYNTSKKWTKLEIEKELENYEKIRREEIFKLFQELEEKILKENKFDGVNHPNFFMQNGNPNFKKYITQGFLIKDKSLKKEDVEWIEGLKDKGSKNTFESNDTIKELQQKPEKIKHAFMLIYLRNKFAHNQLPIKTYFDMICPNFDTTKSITEHIFNYLK

SEQ ID NO: 9 - protein, 945 aa, synthetic construct
MAKKKHLSAADRAAQKAASSAQRSSQSKEEERIRKEAEAAAKAEAEAAERAKKEAQLLSTFKKDRAGEPQKRKSTAKAAGLKSTFVIEDKLLMTSFGRGNEAVPEKRISDDVITDVSSAPAYSVTPVEKRFDICGRANNSGTTDNPVFSKKKSGDDLIRGREALETRFFGHTFSDNIHIQLIYNVLDIEKILAVHINNIVYALDNVRRADYSEDDDFIGYLSTQNSYAVFQNPKGTNKTPGAINSINKSREAFEQFIRNPRLGYFGSAFYNVDNNNDDNNKKANRGKAELKSEEDIYYFLCILGAARQMLIHSNDYTKAFLYNIKDKEKADVKGFLDRTYEERINEVNKSFIENSKKDLTIIFNAFNVKTLEEKASFAHDYYDFIVRKAQKNIGFSIKKLREIMLEMIENADLKSSDYDTTRSKMYKHFDFSIFEYYRKNPKKAEAIVEKLRTAADDEQKERVYVREAMALWDAIGVSFRKKVVPNMTGTAIKDITADPDINDSLFEGVLIGADTDYFCKAIYAFTLLIDGKEINDLLTTLINKFENIASFISVMEHEGIEFRFEKSYKMFEKSAEIASDLRVINSFARMQKPDPSARKQMYIEAAEILGTGDSEEELSEYFDNILDKNKGKKRADGKKDTSLRNFMASNVIESSRFRYLIRYCNAKKVREIANNRPVVRFVLDGIPDTQIDRYYASCKGTGNPDAGKKREFLADMIASMDFRSFENVKQDDRKSSDEEKQEKEKFKTIISLYLTVLYLLIKNLVYVNSRYCIAFHCLERDRQLHGIGKDESLTCLTQLFLDNGWLNEHATEYLKTNIENSDDRVITYYRNSVAHLGAIRNLNEFIGPAKQFGSYFELYHFVMQSALKKQCGLEGYTPGEKTQTYFVLVEKYGSCCKDFIKALNVPFGYNLPRYKNLSIDGLFDMNDAREKEKGKAKDINIKNTD

SEQ ID NO: 10 - protein, 952 aa, synthetic construct
MAKDKKQDRVIGLGALAGAFGMETAEPKKEAKTEKKEKQPVKPVVAELISLEGMEEEKKAVAAEQPKAEIGKKSSSKAAGVKSVFTMGDGSLLMTSFGRGNSAVLEKNVTEDNEINDINTEAPVYSAKVKENGFTVKGRASHAGSVDDPRKSEKRPDQIDCREKIEQRFFGKTYEDNIHIQMAYNIMDIEKILAVHVNNVVYALNNILGDEEAFYADWIGSMGWDKTYEKAMSAKESNSRFVADCFKNLMRSKRRAYFGDVLFDAGISKITKKEIKEARIEEERKKAYYICLLLGKARQMTAHDLADTRSALYCLDETFDAKCKNEAKEVRVEVRAVLDELYYARVSKLNSDFIDTSKKDLVLLFRAFGITEPAQKKEYAGLYYDFVVRKNYKFMGFSIKELREVITKTIPEAAVVKDEKYDTVRQKLNRLFDFAIYDYYRKNPAEADSIVCTLRAVQSETDKQLVYRRSAKQLWKSIRATVLNHILPNMNGDAIKAIVADRDITADALKDVLVSPNADYFVKFLYMLTRFQNGKEINDLITTCINKFENIASFIAVLEEKHIGRRFAQEYSVFERSAKIASQLREMNNFARMSMKSDETATKKAMFEDAALILGYENAAELDSMLDRMLDPKGTEKGFRNFIVNNVIESVRFDYLVRYCNPTKVRKLASNAVVLGFVLRGIPDDQILRYYNACTGSEAKECTPRMRDELTKKLEGISFNEFRDVHQKDATATPAQAQDKVRKQAIIRLYLAVLYLLVKNMVYVNSRYFMAFHCLERDNSLYTGSEINKNDYAAFARAFMQEERHRKNARAARYLDVDFANSDPQAVHEYRNCVEHLSAIRRMDEYIADVGKMDSYFDLYHYIVQRHLSDQHDRAIRENKYGKIENPKMLGFIANMKRYNGCSKDMIKALNVPFAYNLARFKNLSVDGLFDMNDTREKPKGKNCGMETEEAE

SEQ ID NO: 11 - protein, 952 aa, synthetic construct
MKKKENTKQKREQLAAAKRAGFQKQEEDKRKAEALKAEQQKAAEKAKARKALEAQIRAEDPQISDKRHSTAKAAGLKSAFTINANEVLLTAFGKGNAAIPEKWVKDEAISSISADPMFSAELNEAQFVIRGRVNNEGNADDPRKNASAGDDQIGIRRPLERMVFGGEFEDNIHVQIAYSILDIEKIMAVHINNIVYAIDNLQRNEGKDFDDFIGYMGLRKSFEQFMKKDSGTDKELQDIFLRLVKSPQMGYFGDVIYDAGIQKKAVAKIRKKQDIFDDPAVAAHYKKCYYVFALLGMMRQFSSHGTPENRAALYTLDAAYDRSGSDYKEKNIARSEARDALSGMYARRVDELNKDFLDKAKRDFVILFKLFDARTVQEKQHLVRDYYDFIITKSYKNLGFSVKHLRELLETQTEAARFKEKEFDSVRRKLNHMFDFVIFEHYQQHPKAAQALVECLRASTSDIEKEICYREEAVSLWASISEQIMNVLLPKMNGNIIAHIDADGDVQEAMLNDVRISSEATCFTKLIYLLTCFLDGKEINDLLTTLINRFENIASFVSVLRSQKMSCDFAEKYTLFADSDVISRELRLVNSFARMTQENANTKRIMFIEAAQILGYPGSREEMETYIDKLLEKKEKGQNGFRNFIANNVIDSTRFKYLVRYCTPAKVRKMADNRAVVDFVLKDIPDAQIVRYYNGCFAADEVYREGMRDALAKLINGMSFTEMEHVRQGNNLSEEERIFKERKQAIIRLYLTVLYLLVKNLVYVNSRYFLAFHCVERDAMITDAKKYEKELKADNKNKDWAMFAREHVEGRRDGRVKRYLMTNFANSDLWTLRAFRNSTEHLNTVRNAEIYIEELRSFNSYFSIYHYLMQRQLIDQYDWERDKGYISDDRMTGKTLRYFGLVRRHGSYCMDFVKAMNVPFAYNLPRYKNLSIDNLFDRNKPGADDGIGPKAE

SEQ ID NO: 12 - protein, 931 aa, synthetic construct
MMGNKKSVAKANGLKSTFVLGENTAYMTSFGRGNAAQPEKHIRDATVTDIQYTFRAKTDGGRTVHIEGRVGASDVLLPDAANQLHAKDAVEQMYFGKAFSDNIHIQIAYNIMDIKKIFGVYANIIVHTVNNLCCDGDKQDDFLGMFKTQNRYQVAAWAHKIVSLHLVKNELRGGGFFMDQEVWRAHVRTDFKSLNLAVNAFMKKYPQKYPYWSVKIVSDFIVQEMGIKNKVILEKAAESYVEFETVAKRLEKSAYYFSDIFAGKDGKFDEQKAFDLLRVLGMMRQEAFHEKNSSASWLYNLDAEADEDIKAALRTVVDTKVTGINTNFAKQNKVNLLVLQEIYPQKSKADLVREYYDFSVRKAFKNLGFSVKTLRETMCAFDAASVITDKQYDTVRGKLYSLFDFVIYNYCLENEAVCNAFVEELRANLDPENKTALYQTLAEKVWAEIGDIVLHRILPQMHAKKIQEKSKETDAETVEMQGYVQAPQDLSLFSKAVYCISMFLDGKEINSFLSALINKFENISSLCAVLAYNGLETEFVAPFTFFADSQAIAEDLRYIKSIARMNKGKKATKDSPVTVKEMQYFDAAAVLGETDTEKVKAAFHLGDKSASTADKAFRNFVVNNVINSNRFVYVVRFINPKNAREIMQNRALIAFVLKDIPQSQLVRYCGTAGIACNADEPNTEAMVNALADMLLQVRFDAFSNVQQKVKADSAEAVQKEKYKAIIGLYLTVLYLLVKTLVKINMNYAIAFGILERDCQIMNQKHGKNPKRDRDAFYMREQQNKQYVYNARAITELFIENGWLNKRVQKSVENNAALYSDEAFYKYRNLVAHLNVISALPKYAKDITKVKSLFDVYHYILFLSLCEDKNSNLPEAVMKSLCKNGKTMLENAREHQTVCKDFLYGLNTPFAYNAARYINLSNREKFLAGFGK

SEQ ID NO: 13 - protein, 951 aa, synthetic construct
MKKRVNAKEKRELRKQQQKERFRLENEAKTPAAVTEDKKEGNGKAEEKAMIPGISPKKSSAKAAGVKSAFILSENRILMTSFGKGNQAVLEKKIEGERVDDLNDRPVYSAFPDGTVIGIKGWKDGMVDNPLHVNDENITRAPGSDILGLKDKLEARYFPNDAPFDDNIHIQLIYNILDIEKILAPFVNNTIYVINNLTRSEGREMDLIGNISEKNSYDTFMAPETEIFDDTILEKVINQKAYFEKIITMPQRLYLGEALYNTRLKKGQKEDESDRKRYYYTLYLLGRMRQSLFHGDRKKQSMLYTIDEKFDSNSETEDLLKARKEARETLDGIYSVRIKKLNEEFLNLAKKDLAVLFRMYGAKTPEQKEILVREYYDFTIHKEYKTLGFSLKVLREKVFASPEGASVKNSCHQSMWHKFVKNFDYIAYHHYKEKPEEAASIVEALRSSMADAEKDGIYSAAAGRLWESIRPVVTESLLPVMDPERFKHLTEDGDVNKTMLNGVSIGTDVHYFSKLMYMLTMFLNGKEINELLTKLINKFESIDSFNSILSSQGLSLRYEKWYSMFLESGEIAKELRTINSFARMQKEDPDAKKTMYVEAAQIFGFNDTDEALSDYIEYILTKDPMKKAELQSSIRKSSFLLPNEGLRNFIANNVVESTKFKYLVRYTNPAKVSVIAHNDQILKFVLGNIPDEQIERYYRSCIEDASASPEAMRDSLRHLIKGVSFTDFADVNQKSNAEQNEDKRKKQSIIGLYLAVIYLAVKNLVNINSRYTLAFHCFERDYALIKKHGYDKNNESYKCLTEEYIELGKLNKKACRYLKQNLSNAYNQTIRFYRNEIDHLTVLRDFPEYVRDVKKVDSYFALYHYLVQRSLKSEYEHRKLCNNLMDGNEPVTESYFADVAKYGKYRKDFVKALNVPFGYNLPRYKNLSIEGLFDMNYPGDKGCTGPDDSCT

SEQ ID NO: 14 - protein, 991 aa, synthetic construct
MAQKQQKPKNDKRMTGLSGLNALGAAFGLEAEKPTEKAPAVREWKAEPIKAVQEKVTADGALRNRRIKEQKEALAREGVKKHPAKAAGVKSIFTLKEGHLLMTSFGRGNAAVLEKDVNDTVIEDIAKEPAYLAQHAPKRFDIKGRIGIKAKVGNPVCGGRPAGDDLIGLRSRIEQRFFGKNFEDNIHIQLAYNILDIEKILAVHINDIIYAINNMAREEDSEHGDVIGLLTMNKEWANFIKNGGEKAEAVQNLLNKEQLSYFGTVLYDPALVKKLKTAKNDRFREIAKAALDEKKKNAYNLLTMLGAARQSLAHDNPVIYKLDKEYDDQIKNRNDERIKPAQRLAARDTMDALFAEAINGLNSNFLNNSKVDFHILFTVFDAKDAADKTELVRRYYDFVVRKEYKNQGFSFTRLRENMVLSAGAKPLMNTDYDSVRGRLYRHFDFILFNYYLDNADAASAMVEKLRACMTGEEKEALYEAEAKRVWPLIGAKVMNRVLPLLDGTSIKRVSAGSADSDIPLDALAGTAISPADTSEFSKLIYLLTCFEDGKEINILLTTLINKFENIDSFRQVLRELKLPSGFADAYRMFGKSGEIARELRMINSFARMDMKNQPDAKRVLFVEAAKVLGYPDTEEKLYKYIDGMLTATGEFEAKGKDFRNFIASNVIESDRFKYLARYANVNRVRAIAANKNVVAFVLKGIPDTQIVRYYNSCTGADKKEAEPGMREKLTAIITGLSFEQFAGVRTKTNNIDKMQKQAIISLYLTVLYLAVKNLVNVNSRYFLAVHCAERDANLFDRERFHASFEAKGDMREFAKERAGLRRIEEHNAEIEAWAKAKAEREGKPAERKPRERRKLYTEDGRRINVRARSYLEQDYYNGDAFIIREFRNCAEHLGAVRNAADYIKDIGSFDSWFEMYHYIVQRYLEGQYNFYKGQNKVSDEEMTGKTLEYFERVNATQSYCMDFVKALCVPFAYNLPRFKNLTIKDLFDMHD

SEQ ID NO: 15 - protein, 984 aa, synthetic construct
MPRLKAKEKRELQKKNMANRYAKENISVIGTVTKADEVPDEPELDETKEIIKTKASTAKKAGVKSTFILDGNKLLMTSFGRGNEAILEKRIDNEEITDLNNKPQFELGKGSLVNEYSVVGRNRKEGTAVNPNHKNTENDRYQKMPGEDNIGIKGYLEKTIFDNCEDCKDNIHVQIAYNILDIEKILSPCVNNVVHCIANVTRKNKMSEALDLGMDIHEDNPYDALVKPEKYFDEDKTARVTSSYEKFISLISSPRRRYFGEALYDPSEHNEKKWIDTEDDLKRYYYELYILSMLRQSLFHGEVSDSADIYKIDERFDGDYQAKPTRVTARKEAREQLDIIYKKRVDKLNNNFIKMASEKDLPLLFEIYHADKKEEKEKIVKEYYNFVVRKEYKNSGLSIKKLREKILYASEGVLSKDGETIRSAVEHSAWSKFVKLFNFVIDKYYNEHQEELRNVVEELRGDSTDEEKDNTYRIEADRLWQIIREHIMNTVVPNLNESAIKEIKAEKGIDKNILSNIGISGDSNYFCKMIFVLCMFLDGKEINDLLTQLINKFDNISSFNDVMDAYNIKYKYSKDYSIFNDSKRIRIELGVVKNLACMLENDKDKDKKKKGDKTIPEKNGIERVMYLEAARALGVSCDNEELNEYISLIITTDEEDIKAIKERLKYDFKRFPMPKKGFRNFVINNVIKSKRFRYLARYINPKDFELIAKNEKIVKFVLKEIPEKQIARYYQSCLEPGISIDNIKPSDYEMMRNQLAKTISRLKFTDFTSVDQSSNALENEDKSKALNLIGLYLTIPYLFVKNLIYVNARYSMAFHSFERDHKIITGKKFNKHKLTYDVITKKFIEEKRINSRASKYIQTNMEKSNAFCIHKYRVCVAHLLVLDNITKYINDIDTIDSYFSAYHYIAQRFICDQYYSEEGTEYRDANSDAFFESIKKYHCYRKDFVKALNIPFGYNLPRYKNLSICELFDRNNMGKQIPLQQCAD

SEQ ID NO: 16-17 - skipped (2 entries)

SEQ ID NO: 18 - protein, 8 aa, synthetic construct
  VARIANT 3: note = Any amino acid
  VARIANT 4: note = W, F, or Y
  VARIANT 5: note = Any amino acid
  VARIANT 6: note = Any amino acid
KRXXXXAF

SEQ ID NO: 19 - skipped

SEQ ID NO: 20 - protein, 6 aa, synthetic construct
  VARIANT 5: note = K or R
  VARIANT 6: note = W, F, or Y
LGKRXX

SEQ ID NO: 21 - skipped

SEQ ID NO: 22 - protein, 20 aa, synthetic construct
  VARIANT 4: note = Any amino acid
  VARIANT 8: note = Any amino acid
  VARIANT 11: note = Any amino acid
  VARIANT 15: note = Any amino acid
  VARIANT 19: note = Any amino acid
GLFXALLXLLXSLWXLLLXA

SEQ ID NO: 23-28 - skipped (6 entries)

SEQ ID NO: 29 - protein, 16 aa, synthetic construct
APKKKRKVGIHGVPAA

SEQ ID NO: 30 - RNA, 36 nt, synthetic construct
  source 1..36: mol_type = other RNA
gtacagtagccctacagattcgtagggttctgtgac

SEQ ID NO: 31 - RNA, 36 nt, synthetic construct
  source 1..36: mol_type = other RNA
gttttagacccctgatatttcgcaagggtatagtac

SEQ ID NO: 32 - RNA, 36 nt, synthetic construct
  source 1..36: mol_type = other RNA
gtttcagacccctaccatttggtaagggtatagtac

SEQ ID NO: 33 - RNA, 36 nt, synthetic construct
  source 1..36: mol_type = other RNA
gcactacacccctgacttcggtcgggggtctaaaac

SEQ ID NO: 34 - RNA, 36 nt, synthetic construct
  source 1..36: mol_type = other RNA
gtaagacaaccctacaaacctgtagggttctgagac

SEQ ID NO: 35 - RNA, 36 nt, synthetic construct
  source 1..36: mol_type = other RNA
gaactacacccctgcgaaatacgaggggtctaaaac

SEQ ID NO: 36 - RNA, 37 nt, synthetic construct
  source 1..37: mol_type = other RNA
gtgtcagacccctgtcagataacaggggtgtagttcc

SEQ ID NO: 37 - RNA, 36 nt, synthetic construct
  source 1..36: mol_type = other RNA
gttgtaactgctcttattttgaagggtaaacacaac

SEQ ID NO: 38 - RNA, 36 nt, synthetic construct
  source 1..36: mol_type = other RNA
gtttcagactcctgacggagtgtagtagtaacgttc

SEQ ID NO: 39 - RNA, 36 nt, synthetic construct
  source 1..36: mol_type = other RNA
gtactacacccctgggatttcacaggggtctgaaac

SEQ ID NO: 40 - RNA, 36 nt, synthetic construct
  source 1..36: mol_type = other RNA
gaactacacccctgtagaattacaggggtctaaaac

SEQ ID NO: 41 - RNA, 24 nt, synthetic construct
  source 1..24: mol_type = other RNA
tgtagttttacatgggtgtaaaac

SEQ ID NO: 42 - RNA, 36 nt, synthetic construct
  source 1..36: mol_type = other RNA
gaactaaacccctgcggatatgcaggggtctgaaac

SEQ ID NO: 43 - RNA, 37 nt, synthetic construct
  source 1..37: mol_type = other RNA
gttctatacccctgtgatttaccaggggtctgaaaca

SEQ ID NO: 44 - RNA, 36 nt, synthetic construct
  source 1..36: mol_type = other RNA
ggactacaccttggcagtaatgctgaggtctgaaac

SEQ ID NO: 45 - RNA, 37 nt, synthetic construct
  source 1..37: mol_type = other RNA
tgttgtaaatgccctaattttgaaaggtaaacacaac

SEQ ID NO: 46 - RNA, 36 nt, synthetic construct
  source 1..36: mol_type = other RNA
gaacgttactactacactccgtcaggagtctgaaac

SEQ ID NO: 47 - RNA, 30 nt, synthetic construct
  source 1..30: mol_type = other RNA
cacccctgcgaaatacgaggggtctaaaac

SEQ ID NO: 48 - RNA, 30 nt, synthetic construct
  source 1..30: mol_type = other RNA
cacccctgtagaattacaggggtctaaaac

SEQ ID NO: 49 - RNA, 30 nt, synthetic construct
  source 1..30: mol_type = other RNA
gacccctaccatttggtaagggtatagtac

SEQ ID NO: 50 - RNA, 36 nt, synthetic construct
  source 1..36: mol_type = other RNA
gttctatacccctgtgatttaccaggggtctgaaac

SEQ ID NO: 51 - protein, 1159 aa, unidentified
MKVTKVGGISHKKYTSEGRLVKSESEENRTDERLSALLNMRLDMYIKNPSSTETKENQKRIGKLKKFFSNKMVYLKDNTLSLKNGKKENIDREYSETDILESDVRDKKNFAVLKKIYLNENVNSEELEVFRNDIKKKLNKINSLKYSFEKNKANYQKINENNIEKVEGKSKRNIIYDYYRESAKRDAYVSNVKEAFDKLYKEEDIAKLVLEIENLTKLEKYKIREFYHEIIGRKNDKENFAKIIYEEIQNVNNMKELIEKVPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQDGEIATSDFIARNRQNEAFLRNIIGVSSVAYFSLRNILETENENDITGRMRGKTVKNNKGEEKYVSGEVDKIYNENKKNEVKENLKMFYSYDFNMDNKNEIEDFFANIDEAISSIRHGIVHFNLELEGKDIFAFKNIAPSEISKKMFQNEINEKKLKLKIFRQLNSANVFRYLEKYKILNYLKRTRFEFVNKNIPFVPSFTKLYSRIDDLKNSLGIYWKTPKTNDDNKTKEIIDAQIYLLKNIYYGEFLNYFMSNNGNFFEISKEIIELNKNDKRNLKTGFYKLQKFEDIQEKIPKEYLANIQSLYMINAGNQDEEEKDTYIDFIQKIFLKGFMTYLANNGRLSLIYIGSDEETNTSLAEKKQEFDKFLKKYEQNNNIKIPYEINEFLREIKLGNILKYTERLNMFYLILKLLNHKELTNLKGSLEKYQSANKEEAFSDQLELINLLNLDNNRVTEDFELEADEIGKFLDFNGNKVKDNKELKKFDTNKIYFDGENIIKHRAFYNIKKYGMLNLLEKIADKAGYKISIEELKKYSNKKNEIEKNHKMQENLHRKYARPRKDEKFTDEDYESYKQAIENIEEYTHLKNKVEFNELNLLQGLLLRILHRLVGYTSIWERDLRFRLKGEFPENQYIEEIFNFENKKNVKYKGGQIVEKYIKFYKELHQNDEVKINKYSSANIKVLKQEKKDLYIRNYIAHFNYIPHAEISLLEVLENLRKLLSYDRKLKNAVMKSVVDILKEYGFVATFKIGADKKIGIQTLESEKIVHLKNLKKKKLMTDRNSEELCKLVKIMFEYKMEEKKSEN

SEQ ID NO: 52 - RNA, 36 nt, synthetic construct
  source 1..36: mol_type = other RNA
gtgtcagacccctgtcagataacaggggtgtagttc

SEQ ID NO: 53 - RNA, 36 nt, synthetic construct
  source 1..36: mol_type = other RNA
gttttagaccccttcgtttttggggttatctatatc

SEQ ID NO: 54 - RNA, 36 nt, synthetic construct
  source 1..36: mol_type = other RNA
gatatagataaccccaaaaacgaaggggactaaaac

SEQ ID NO: 55 - RNA, 24 nt, synthetic construct
  source 1..24: mol_type = other RNA
agttttagtcccctttgatattgg

SEQ ID NO: 56 - RNA, 35 nt, synthetic construct
  source 1..35: mol_type = other RNA
ttttagtccccttcatttttggggtgctctaaatc

SEQ ID NO: 57 - RNA, 31 nt, synthetic construct
  source 1..31: mol_type = other RNA
ggccaccccaaaaatgaaggggactaaaaca

SEQ ID NO: 58 - RNA, 36 nt, synthetic construct
  source 1..36: mol_type = other RNA
gttttagaactgtgtagttttacatgggtgtaaaac

SEQ ID NO: 59 - RNA, 36 nt, synthetic construct
  source 1..36: mol_type = other RNA
aatgtgaatgatcccaatattgaagggaactacaac

SEQ ID NO: 60 - RNA, 36 nt, synthetic construct
  source 1..36: mol_type = other RNA
attgtggattatcccaatattgaagggaactacaac

SEQ ID NO: 61 - RNA, 36 nt, synthetic construct
  source 1..36: mol_type = other RNA
gaactatagtagtgtgaattaacactactctaaaac

SEQ ID NO: 62 - RNA, 36 nt, synthetic construct
  source 1..36: mol_type = other RNA
gttttagactactagcattctttattgttctaaaac

SEQ ID NO: 63 - RNA, 25 nt, synthetic construct
  source 1..25: mol_type = other RNA
attactgcacaggggattctagagc

SEQ ID NO: 64 - RNA, 38 nt, synthetic construct
  source 1..38: mol_type = other RNA
gggaactacacccctgctaacctgcaggggtctgaaac

SEQ ID NO: 65 - RNA, 36 nt, synthetic construct
  source 1..36: mol_type = other RNA
gttttagacccatgttatttagcatgggtgtagttc

SEQ ID NO: 66 - RNA, 36 nt, synthetic construct
  source 1..36: mol_type = other RNA
gaactacacccatgctaaataacatgggtctaaaac

SEQ ID NO: 67 - RNA, 36 nt, synthetic construct
  source 1..36: mol_type = other RNA
gtgtcagacccctcaagtttctgaggggtatagtac

SEQ ID NO: 68 - protein, 1339 aa, synthetic construct
NPSRVKIPLCDKCSSASGQNNTKLCDSCQKRQSAAKDLAVEIKQIVNQIICEELANQNKDFGKKVINSKANSKDGSKKSKEYNIKMEDLDLLIFCLKKKFRSNVKFQDETNVDHKFCFLTDLGDYLRSELCLSPELSQSSTPSTSSPSSKHSSFLDAYRQWVRSYVRKKKKYTKRSIENNKLVVDYSDEASLSPRKKCLLKWGDEYRKNGKIDLRTEEKLLGMDELIESINKVRKDHGEPIIDDARGNEQMNYEFNVAVKRAIQSRKKAVFGLVKESSLDNGSNLTNKINSTNEINSINERNLENRKNANLTTYFNEVVKYFEHYFPIKSKKYDIIKNKKGKRKGEELKKSLRYLEADTIKSTVLSQIQNAVRSQFLQQGKSELYDLKKETTCSDQLSLLKRDEFFVLNLIEACAFATNNIRNIVGQTQCEDNQRKDNQRKDIQREDILGNDITKGVIDCILKNEKDPSPNKSVINSASNSIFNKELFNSFYGRELSVKEEDSVSQFVKTIWAMRGSVQQIRNEIVHFHMGSIDKIFNLENFEYFDQKSDSNQTESSKLIVSSVSSEPTQSTQSAQSTEKSNEPNRSNEPYTETVFKDCLENDLNRVSELFIEKLQSNDVLNYYRAHDIQHLLEKLEFSLCKCSIPFAPGFKNIYEAGKYLQNAGNQHFINNQQSINNQRDERTKRSQSNECNQSNQTDVENNPFELRHYLLKDKDAAKTAQAKNYSAYRFLLKILYDYCFLYQFLNDKQAFEEAVRFTLDNNKAANSRKANKNEFAFNGIPYFDPSQSIEAYFSEIHSLAVHEENKKKEKDGEQRLNTQRFITKVFVKGFDNYLKEIKVGFLLNSNQDQPKKLTQEDYSPLAEFVKVKSCIDKKQNSHIAFYTFCKLLDAQHLSDLRNEFIKYKASNRTTNTETSKPNDKRKFDYIIDILGLCLLSVDSLKCNDLNQRMDECTKQSWKSDLESFVDADLLSFIDRGQPDSEVINLYKQSDDYTPVAHASIRLLRKYGTKELLKQIIGTDYIVSVGDYNEWQKFKDIAVNKECTVIEDYINTRTRLHDEWKSLVQEKKNQAATRGKGKYKPNKDKSNKDNEAIISKSTIDNFILKNKAEYVDVCNKIERYNWLDNKIHLVHLRQLHNLMIQILSRLTRFVALWDRDFVKFDRTFTDSQYIFTGVVSLASICNGKESNLEDKVKSRSDNEPYKSKDILSLQTDTKAKREVYNNLFFKQNKDAFSDRNYIAHYNYITKGGTKSILDLIKRVKKLMGYDRKLHNAVVKSIINVFDQNGMELRMSIDSNHELTLEENAIIPKTINHLERTDISTNLVSEEYCKLCQKMLEMKK

SEQ ID NO: 69 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELHPYYDWSKWYINDKSGKLKKSITNNHIQIGDGESKRQKALLMWETDFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRKGKYQHHDLKADATSENLTNLKRDEAFVMNLITTSAFAANNIRNIIDPEQIKDIVGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSGRYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRAASESELFIHLTEIIELCLLHADNVPADYWQIYKNKEQCLQRLQPFIAENSDYTNWGDLYVQTDDETPVVHSGIELSVKYGTASLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITSKIIFHLGTKKESDNIYTFQVPTAYCNMCKQLLEMKN

SEQ ID NO: 70 - protein, 933 aa, synthetic construct
LKKKVKARDLREAKKQEKIAVYSAAAKAAVGDKSAVEAKPVRPVEAAHEDKAVGNKKSVNKAAGLKSTLVYGDKLYMTSFGRGNSAVVEQRIDTSDYSVSDMNASPSLRVSEAGKINVSFSSLRQFVNAGQLSAVNPLYGGKDKPQKKTGQDMLGLKDKLEERYFGMTFNDNIHIQIIHNILDIEKILAVYTTNIAVALDHMVDEEIDAEREEFIGYMNTLNTYEVFMNPEKKFGAGDKRVKNITESRDKFEKLLATKRMGYFGFDYDSNGKDKHKNEELKKRLYHLVAFAGQLRQWSFHGDTGINPNSWLYRLDDKKLDQEYRDTLDYYFDIRFDEINEKFVEQNATNLCILRELFPNDDFKAVADLYYDFIVIKAQKNIGFSLKKLREKMLESEEATPVKAQDMDSVRSKLYKLMDFCIFKYYTEHPEISNKNVDILRASVSDEQKESFYTDEAYRLWGIFGGQFLSFCSRINKWVTGEHQKEVLRYIDKDAYRRQSSVSYFSKLLYVMCFFLDGKEINDLLTTLINKFDNIASFINTAKELGINVSFVKDYEFFNSSDRYVPELNIVKNISRMKKPAESVKKEMYRDALAILGVPSNMSREAFDAELEKILEKKIDPKTGKPIKGGNPFRNFIANNVIDNRRFIYVIKFCNPENVRRLVNNINVTEFVLRTIPEAQIDRYYKSCIVSELNPVPDKKISELAKMRKDINFGEFRGVQQKAHTREEALEKERFKAVIGLYLTVVYLLVKNLVNVNARYVMAFHCLERDALLYGENVGNDYLNLSETLCSENENSRSGYLARNRRLRECVKQDISNAHTLKVRNYRNNVAHLSAVRNCAEYIGDITKIDSYFALYHYIIQRLLAKGTDRTAADFESRYPYFAQLFKWNTYVKDMVKALNAPFGYNLPRFKNLSIDDLFDRNEQARKKSEMLDK

SEQ ID NO: 71 - protein, 775 aa, synthetic construct
MKKSNSKKMGIKIKTNNKNDIYFFDNKDFKKDNQNYLMLSDEENKLIKKSNTAQFDFLDTHTNKRIDIIKGKNIAEYENKYSINKENLLNQCLIKKYCYKSDVSSNLVIQLIHNNEDIKKIVSLYVNDVIYSLSSINKDNYDVISKISPSFNMKNNKIYTNSGIYNNSDLYNILNNLKYRQTLYGLENKNKAILFEYLKAISSLRNLVAHFNKTDVYNLNLLKNELSNYILIKGKEFLKNSALNIDLITKLYSKENINEEVKQYYLYVLNQKDKNIGLSIKKIMEIARNIYPSNIENEEKNKKYNVLLKYHLFNYLTNNKELTNEYIDKLRKSDYEDKDNIYYELFKKIGKKEFDRVENIIDKIDFKEEVSINLKDIEKEILNVKYNWNYFILQIYALSKFLTIKETNVLTSSLINKFQSMLDLYYVSKSINFNIKDCIKTNYLICSVDEMEDVINQLRIINSSRRYINIKKSKCDERFVIDGLKIFKNNYNNEELKKLLNEKINNKIYKSKKIKNPLKNFIRNNVIKTKEFNYLIRYSDPGKLSNILYKNVNLVKFLFEQFTDSEKEKYKLNNVNEKTISEITLNEIIKNLKSGNMDYSINVKAYVKCIYFLIKNMVNINSYYFIAFSFYARDMMLINSDYKEYSLKLFNYVKSKDRDETRMTIKNFNSIKDKNIFDRYRNIVEHININKLLYDTKFPDSFKYSNYFKIYHYCIQKELLNIEYNNKFLNECKSNINEYNKYSKNLLYVLNSIFIYNYARFKDLSVEEIFMRKYN

SEQ ID NO: 72 - protein, 956 aa, synthetic construct
MSKEKARDKRLRQQEEKKQKIQSVNEKREAERLRKEQEEARAEKAKEEAREKAQFENEIAQIEREAPNSAKRKKSEAKAAGLKSTFVLAADKLMMSSFGNGNSAIREKYIEDGHISNISEIPVLDVKNDEDEKKFLISGRLVSNATADNPLKTKRPTGMDQIKCKDKLEKLYYGKCFDDNIHIQLIYNILDIEKILTVHVNNVVFELNNILRNEGNEFSDLIGEGLGREKTYADFVKSGAKKFDTFKKFAYCKQLGYFGGTLLIPRERDRKKVEDVDNVHLKRTYDLLSALSVLRQATAHGEASTRAQIFNFDSEKSKKSKYYLEIRTALDNLYEDRVHTLNERFLERAAKDLKIIFDIFSVESASEKQRVIEEYYDFVVLKSYKNTGFSIKHLRELIIQNYAGNIADHEYDSVRPRLNRMLDFIIFEYYAKTENQESASYLVDSLRAADRDSVKEALYRRAAGHIWKAIGNAVSDRLLPKMNGDYIGALSEVLVDDMSISSVAIPETAHVFTEMVYLLTIFLDGKEINDLLTQLVSKFEIIESLNAVLREEGLETGFCSDYTMFEKSGEISRQLRAVNSFARMSEEDPSVKMVLFVEAALILGYSGNREELECYVEELFNKDGGKRLTDGRKDNGFRNFIINNVITSDRFKYLVRYGNPEKIRKIANNRKVVEFVLKAIPDAQIRFYYNSCNGTNAAYESAMRDDLADKITGISFLDFENVHQNDKGYNAWHGDKERKKNIIRLYLTVLYLLMKNLVYVNSRYYLAFHCAERDAMIWDDKKYKFTNESKCADRVNFAKAWIEENSRDTRAKHYLIDNFKNADIWAISTFRNCAEHLNAVRNMDSYINDIAHFDSYFELYHYLVQRQLMKEFDYETTTPAKDNNNRMLLEADELTGKTEKYLDIVRKYNFYCKDFVKALNVPFAYNLARYKNLSIDALFDRNNYLGATKESIKSEE

SEQ ID NO: 73 - protein, 951 aa, synthetic construct
MVKKKLTAKERREQQKNDQQAVRAEREAQKLARAAAAEEAGRRAAAEAEAERLRKEEEARLAHVAELRAQAKANKERRKSTAKAAGLKSAIYVNGDELLLTAFGRGNEAFVSKRVKGSEITEAAGGRASYTAEMGDKQFLLSGRTQGQRDDPRSSAKKSGDDMIGARAALEKRYFGHACEDNIHIQIAYCIQDIEKLLAIHTNNIVYELNNLRRNPDAEYDDFIGYLSVRDEFAVFMDDNRRFADKDKDVKKNVENSRQNFRDFIKNPRLGYFGSAFVDVGTDTRARMKSEQKPDADIYATLCLLGTLRQCMAHSYNSSRSDIYRIDKKDGAAWDEARSLLDRTYNARVEALNRDFLSRAGKNLTILFGALGADTLEKKTAFVRDYYDFEVRKSAKNMGFSLKGLRERMLLLPDAAVNGEPVRTHKKYDSVRSKLNKSLDFAVYEYYKGNPARAEDMVESLRAAQCEAEKEELYAAEAKRLWKEMKPVLFDHILPAMDGDRIGKMKPDPEITADMLQGACIGTDASYFSKLIYLVTLFQDGKEINDLLTQLLSKFENIAAFLTVSPLCTAGDFTNDYSFFAKSGEVAGELRVINSFARMELPEAAANVEMYVEAAVILGAPGSRDELKEYFKNLLDKNGGPLKPNGKKDNGLRNFIASNVVSSRRFKYLVRYINPEKARALARNRTVMDFVLNEIPDEQIARYWNGTRSANAAYDESMRADLAGLITGMSFSEFEDAAEYQAGRTDEQRSRKEQQQTVIRLYLAVLYLLVKNLVYVNSRYFLAFHVLERDAQLLGSKAGYNGTPYAALTRWFVKENRLNRHATAYLTVNLEHSDDKCISWFRNKAAHLDAIRNADKYIADVREVTSYFGLYHYLMQRALEDKYIHSGKEIPLYTRTYLNKVRTYKTYCKDMVKALNVPFGYNLARYKNLSIEGLFDRNRPGDGKEDPQVSE

SEQ ID NO: 74 - protein, 910 aa, synthetic construct
MSKKNNPRELREARKAKMKEKYASKAVEKKEVIEEKAAAEPKSDKNKKSLSKAAGLKSVFYAGENLYMTSFGRGNTAVVEQKINTSDYSFSSMNKSPNLRVNNADEINVSFSSTRPFVKENKLTADNPLHEGKSSKPKATGKDILGLKDTLEKRYFGKVFDDNIHIQIIYNILDIEKILAEYITNATIAIDYLCDTEANGVYGDFIGYMNTFNTYDVFYNPSKNPRLTDRDIKNINRSRTNFEKLIKSNRLGYFGFEKEDKKRLYHLMALAGQLRQWCFHSYGNAARTWLYNLDNRNLAAEYYDTLDYFFDERFKEINNDFISKNSVNLFILKDIFPEEELTEVIKLYYDFIVVKSYKNMGFSITKLREKMLEQTGAVAITSKDMDSVRSKLYKLIDFCIFYGYYKDEKKSAENVDYLRSCLNEDAKENFYAIEAKKLWEKYGNLFLDFCNIVSETNIKNLSNDVTKKKHTEILSRYIDFEEYRNSSNVSYFSKLMYVMCFFLDGKEINELLTTLINKFDNISSFIATAKELDIDVNFVADYSFFNDCGKYVNEINIVKNISRMKKPSAKAKKVMFRDALTILGMPHNMTEEQFEAEIEKLVEVEYDVTGKKIKKPHDYRNFICNNIIENRRFVYIIKFCNPGSVRKIADNTAVTKFVLKRIDEKQIERYYISCMEAPDVNASLDYKINQLAEMMKRMDFGQFSNVRQISSNPSEKQRKERYKAIVGLYLTVVYHLVKNLVNVNARYVMAFHSLERDAALYNYKLEKNYTGLAKLLCDEEENSRSGYLARNKRLRECVKQDIENAKELDVQNYRNNVAHLTAIRRVGDFVGDTTKIDSYFALYHYLMQRLLLEKSNADHKYNKQLNQWHTYVKDYVKALNSPFGYNIPRFKALTIEQLFDRNEQKRTLSE

SEQ ID NO: 75 - protein, 911 aa, synthetic construct
MSKKKSPRELREARKAKMKEKYASKAVEKKEVIEEKAAAEPKSDKNKKSLSKAAGLKSVFYAGENLYMTSFGRGNTAVVEQKINTSDYSFSSMNKSPNLRVNNADEINVSFSSTRPFVKENKLTADNPLHEGKSSKPKATGKDILGLKDTLEKRYFGKVFDDNIHIQIIYNILDIEKILAEYITNATIAIDYLCDTEANGVYGDFIGYMNTFNTYDVFYNPSKNPRLTEKDIKNINRSRTNFEKLIKSNRLGYFGFEKEDKKRLYHLMALAGQLRQWCFHDANYKSNDINKEEYVKGKIRTWLYNLDNRNLAAEYYDTLDYFFDKRFKEINNDFVSKNSVNLFILKDIFPEEELTEVIKLYYDFIVVKSYKNMGFSITKLREKMLEQTGATAITSTGMDSVRSKLYKLIDFCIFYGYYKDEKKSAENVDYLRSCLNEDAKENFYAIEAKKLWEKYGKLFLDFCNIVSGSNIKRLQNCDIKELDRYIDFEEYRNSSNVSYFSKLMYVVCFFLDGKEINELLTTLINKFDNISSFIATAKELDIDVNFVADYSFFNDCGKYVNEINIVKNISRMKKPSAKAKKVMFRDALTILGMPYNMTEEQFEAEIEKLVEVEYDVTGKKIKKPHDYRNFICNNIIENRRFVYIIKFCNPGSVRKIADNTAVTKFVLKRIDEKQIERYYISCMEAPDVNASFEYKINQLAEMMKRMDFGQFSNVRQKTSNPLENQRKERYKAIVGLYLTVVYHLVKNLVNVNARYVMAFHSLERDAALYNYKLEKNYTGLAKLLCDEEENSRSGYLARNKRLRECVKQDIENAKELDVQNYRNNVAHLTAIRRVGDFVGDTTKIDSYFALYHYLMQRLLLEKSNADHKYNKQLIQWHTYVKDYVKALNSPFGYNIPRFKALTIESLFDRNE

SEQ ID NO: 76 - protein, 987 aa, synthetic construct
MKKKVKARDIRETKQNKKMNEFADRDKALREAEAAKKAAEEKLRAEKELQEKISRIRVLQSNTKASPFTRRKSNVKANGLKSVLFMDDEVLMTSFGRGNKAIEEIRVQDGKMTRLNDTPVFDAIDQTSHYDVKRTVRIDADPGVISKHEVSEKRAQQLPHADLIGAKDILETNFFGAPQAGNIHIQVIHNILDIEKQLSGFVNNAIYSINNVIGKVTEEGYDVDMIGSLGVTGGGATGKDWFEDFCANRRLLYFGPVFYEGCAGKKENKKKETLRAKEQVAAILELLGGMRQSLTHGVGSKNGSHWLYSQDTQLQPRHKDTLDMFYESKVSELNDNFLKQSGKDLTVAMSLMGAATMEEMTRVAEDFYRHVVRKDARNLGFSLKKLREILIDTRMPQLRSKDYDSTRSKVNRMLDFVLFRYYVREESGIRALELFVDRLRGLSQEGDPETLEKKRAMIYLEEAELTWPKVKERFENTVKKLGDGSRIAQMTVDPAISGIDLSRVRVSAANTSYFAKLLYMLSMFLDGKEINEMLSSIQNKLENIGSFMAIIKRHGLKDLSGTRYELLMDADRLAEELRVVRSFARMERPAPGAKESMILDAGYLIGQTVDNETLRAVFGDRYVNARGKKDSELAAEYPQFIKRNGRFDRNRFFANDTRLRNFLIKNVVESDRFRYIIRYANAKKVREFAQSPALVDHVLRRMPETQIDRYFKACFPGYDNSNREDSIYALRETIRTLKFDQFKELRIDDEEAGERKQRFQAVIGLYLTVLYVITKNLVNINARYTIAVHCWERDHAMVLGLTNMFGRDYEGSHEDIYGMTDRYIERMEERKAECVRKLRAHENGAKRFNGHALNYVKANRSVWNDTEAAYHMFRLYRNAIAHMMAVTNAYKYIPEMKKVDSYFAVYHYAMQRYIMDHGGYIANRIEERSETAKNYTEAVRRYHSFNKDWLHAVNAPFAYNLPRYKNLSIEALFDKNEASAESDGKEV

SEQ ID NO: 77 - protein, 1492 aa, synthetic construct
MKIAKDNGRKTCVAKKWNQNQIKGMIYKSPKPGVLSMDSLHNELKKKDGSVKFLFSIFNTIQVGQEPHKPNKDNYVDDPEKYAKKEKNYNVKMENYNTRKLYSKYVDMIRKEVNNVLINGKKYNDKRFKSNDEIFRSIRNANIDREIKFKTTTLVDIALRKSFKEYREEAICFIECIRKREINDNEKQFISRFLDELRKDFNRLDTSPLFSFLDEKVTESNNGTLIDNIKTVSRITNKILTNPKSFKSNEKIFEGLKNASIDSNINPTELIDNALLEDYKEYKEEARCFIKCIGKKDIAENEKELITQFIEKLRDEYDNNAYSTKCNGTRILNSIECQNTVVQLNNNVVSLPLADNNRLKKNEKDGLNKFLLEYAVLAEDDRMEMLRKLRRLLDLYFAVPDEYDNEASYTLPDEIDTGDFDVWDRHSEGKREDKDFVRVPEELKEANEENKTLEILDSKKLKYDLDNSIRRKNMNGYRFAHKIVGVTPDGVFYDEKNINDMWIHHIENAIERVLRHRVLDNLYKLKVGYLREKIWKDMLNYISIKYIAVGKIVFNFAMDDLGNEDGDCTLGENTSLEGITSFDLEAIKAQETMQRDLAVNVAFAINNFANATVDLDEHNSDLLTVRIKKDNKMKEGEKILEELLKYNGNEEQIRKSILQFFGGYSSWIESDAINKLNDISVCKLIDGIRNAMYSMRNRSFHFKTFDEKKYSEEIQSLIGTIFEEDAKKCVIQEKNKFYSNNLHMFYKVSDLENTLDALYNDVHTRSSQIPSFNSVIVRKNFKAFISTELGIKNNSFESSDDELKWQNALYYVFKEIYYNKFIHDNSIKQAFEEKLNDYGKTIKDDSESRAYDNFKTKIRRLKKYSFAEMCQNIMTEYNLQNNQRRTVSSSRDSLLEKDIYMHYPLLLKKIIAKTFASYIKNSNEYGYLLNPSTHISEIAIEDFLPDWQSEKYKQLISELSKNQELQAWYITGKFLNGRSLNQLIGSIKSYIQYIDDVRKRAKIAKNKINIRFNNENEKKALEVLEVCLKMSGAVSHELTDYFKDEEEYASYLSGYVDYTSYSDHMENHSSALVAFCNEHKCDVYADGKNPIVQKNVIFAKMFGADSVLSKCVAKVTSDNLDTYYEKKENISNYKVKGICSDSDEQRRLIEYQQLANKIELRNLMEYSEIINELLGQLNNWSYLRERDLLYYQLGFHYMCLQNDFKKPESYDCIKRENDTSLKGAILNQITGMYINGIGIYNSKGNIIENSSLGGAGKKIGCFLQYGNELLGDKKGEDLYKAGLEIFEVYKEHANIINVRNDIEHFRFYSSDTISIMGLYSEVFDRFFTYDMKYQKNVMNMFSNILLKHNILPYVKFGTGVKKVGKEDKEAARLIVTMLESERFKYKVDGGIVVNADAKNKAFLNDVFSILSYNDNYIIKTDAKLRTHDVKVDKEKPNKNNNENDNNRNHRKKKNRGEREYKDKESNSFMGGVFDNFDLESAGFKPTNTKKKK

SEQ ID NO: 78 - protein, 1258 aa, synthetic construct
LKISKESHRKTGVGVSEQTGTIGLLYKNPLDKHEKIEDVVKQRANSTKRLYNLFGTGETSKDISKNAKEVAKVVNRAIGSLKFNMKNNQRECIDKELKSQQVISAIEKSKKYDFKENDVDEALDRLLKKSFRQAKSKNALRKLLMSTLVDNKALSTDDIATIQNDFVKKLVADYNKSNIKNNIPKALRNQNMVVQPDSSGILSLSQTRTDGKNNKTKDIKISEKDAFRQFLSDYAVLDDEVRHQLRLKLRRLVDLYFYGEDTFIKDDFDEWKVHEDNKSNTEPFVTKVTVKKNTKDNKEFEILNVDATADVIRSENISRYRQALDYVNSNKQFFFTDEMLNKFWIHHIESEVERIYKRINAKTGDYKFQRGYLSEKVWKGIINYLSIKYIAEGKAVYNYAMTALSLDNDSKEFGKLDERVSKGISSFEYERIKAEETLQRECAVYVAFAANHLANATVVLDDVNTDFLLLKNKELDAKAIEKGEKSLAECAKAKPDTLRNILQFFGGQSSWKDFDFTGCDEIQLLDDLKEMIYSLRNSSFHFKTENIADNVKNKDLINAMFAFECVKAGTVQKNKMYSNNVSVFYSETDIEKLLGSLYSRINERASQVPSFNTVFVRKNFPEYLKNQKLTPAFTQDDILKWQSAVYYLYKEIYYNGFLQDKTAFDLLTKYVRSLDENCADKQQASANKDFKVAFELYSKSGSLSETCQMIMTEYNNQNKGNRVGISARTQKADRLIFQHYKMILFAGLQSAFTEYIKAHSDIYGFIFKPAVKENIPEVESFLPNYRTNQYNELVNLFKEEIELQKWYITGRLLNSKQVNQLIGSFRSYKQYVYDVVRRAKENGNQLINAEISVDADNIIKVLDVCTKLNGVTSNCLEDYFADSDEYAKYIGRFIDFGLKKGEELANEKLASFCQRELGDRKVGTYHDGINPILNRNVIQCKLYGASDIISKAGILPVDYEIIKKYIGMTKDINGYQESGVCVTKDDQQKLKKYQELKNMVELRNIVEYSEIINELQGQLINWGYLRERDLMYFQLGFHYLCLKNSSDKPEGYDKAGEMIGTILYQIVAMYTNGLSMVDAKGKSKRNAKASAGKKLESFFSYSAEMTDNKYGLYLAGLELFENVSEHDQCVKLRNDIEHFHYYAVQDRSMLDMYSEVFDRFFTYDMKYAKNVPNMLYNILLQHLVVPNFQFGTGKKQVGDKNKQTKSRATILLAEKNGLASEKFTYKLDGGKSTVMLSAKSNDFLSDVAAILYYPDSAP

SEQ ID NO: 79 - protein, 1080 aa, synthetic construct
VEGFFIDIPNELNSEKQPDAVKKKELTESFRADIRSKNIACYRYSLAVIENDTEGLFFSEKEINKFFVHHIENSVERILNNRLLDNLDYKLKVSYLCEKVWKDIINYFSVKYIAFGKAVFNFGMEELLSNDAEISMGIIPDEIKKGLNSFDYESIKARETLQRELAVNVMFSANNLARATVDLSEKSDFLLYSYDKDYKKDPENKKSDIDKYRLSDNDSETLRSILQFFIGKSKWDEKLFLDTYGSEYASEFLNDLKNCIYYLRNESFHFSSAKSSFNWNKNLIGKMFEQEANACFIVEKDKFYSNNLPMFYDIDYLRRALAKLYANDVPRASQIPAFNNVCVRKNFPKLLEEKMKLFALANISDADLEKWQSALYYLFKEIYYNDFIQDEKSKGMFISAINNIKQDVEKRVEDIRKRLVIENSDLNDFQIKKKLDKYPEIAEKNAVNNFCIRLNEIKQDNDLSQICQMFMTEYSSQNNKRTVRSSKDSILDKEIFKHYKVLLMHGLENALVSFIKSKDEYAFILKKPVLREKPSAEDFLPNFTTNKYDCLIKAVKNDPRLQSWYITGRFLNGKMLNQLVGSMRSYIQYVESVERRANNTQNVLHKDETDMKNYIKEACKVIEICILLSGTFTNNISDYFKDDREYSDYINYIHKYVDYEDLRQGEIFFNEGGKPIVNRNVIMAKLYAPDTVLENCVKKVNDEDIKNYMLMSGEIDSYRVKKEITSKDEQIKILDYQKIKNKVELRTLVEYGEVINELLGQLVNWSYLRERDLFYFQLGFHYACLNNDSAKPDEYVSLSYKEGRVINNAILYQIAAMYIYGIKLFAPKKNELQGKFDAVTGQAGLKIPAFERFSERTTGDKDCLYNAGLELFEVINEHDNIIEIRNYIDHFKYYNTLSDTKSIVDIYSEVFDRFFTYDMKYQKNVMNMLGNILLKHNVIPEFVVETGKKKVSKDGEKLRANIEVRSLEADTFTYNYLERGNQVSLIIAAKNKEYLETIAKILYFPEKKSTVKYIIKEADSVKVNGENSRTEKTNGKKSKDNKSKDNKRNNNIKVGDAYKSYSESESCGSSMADLLKNIKL

SEQ ID NO: 80 - protein, 1093 aa, synthetic construct
ESLEIIKNDKERLFFDDFELNKCFVHYIENFVEKRFENIRCSKLYMLETGNISEKAWKSLLSYLTVNYIAIGKAVYNFSMSDISKTEGPMNLGVVEDKWKTGISSFEYEKIKAEETLQRDIAVYVSFAVNNLSKATIKPEKGKEDILLLGDEELKSVLLSSLSGLTEEDALLRNIMMYFGGISIWKEFFENRFSNEYSKFDFYKDIIHILYAVRNESFHFTTANVIEDKWNTKLISDIFNNECDSISRVQKDKFYSNNLHKFYSEDKLKKVLNFLYDEYTIRPSQIPAFNKVFVKNDFSAYLDANFVTSYSWSDSEKKEWEAGLYFLFKEVYYNRFLRDITVEKFCETVKQLDILGEKQKFAHKNFNSRLEKLKKEGFSLPEICQILTVEKNLQNKNRKVKTSKSEKENPDIFKHYTEILESGLRQAFTSYVNEKLSFLNETKRNSNVDSGEVFKTEFLKDWKGKKYDSLKAKISQETKYQKWYILGKFLNPKQLNLLNGAVKHYIQYSKDVERRAKQTGNPLSADSSSHRKECEIISEILDMDMQLIGKVFRDNYNEENNSLSPDDVILDYFDSTDDYAKYVSLFVDYGYESNKVDDDATRPMGYLQAFCNTDTSDGQKIGIYYDDKKPIPNRNIIISKLFGLPFQNDKFKVLNRVTEHDIREYYYKKDELKEYKKTGKCNTFEDQKKLKDFQELKNRIELYSMVDYSEIVNELLGQLVNWVYIRERDLMYFQLGFHYLCLKSNCKFPEQYKTITIGNQGTINGAILYQLVATNTNGLPVYTLDDDGGDVDAYKYANKSTGAKISKCFLKYSEKIGYSEENNIYDAGLELFQNMSEERDINEFRDYIDHFKYFVDEKRSILDLYSEMFDRFMAYDQKYRKNVPNMLTNILKRHFIITHLAFKSGEKEVGGGNKSTKKEMGLICIRDNGIESDDFTYKLADKSTVKLPARGTNYLDGVAKLLYSPQSPPKKALAKTQEAKVASKNVNPKIGEKYQGVVKNINKHGIFVDFGPKKEGLVRYRNIPMKYKHDSTIVGLDKGDTIDVKLLDINEKGYDLKILEDGEFEFEKYKSEKINNTMGSVFKGIDLSFLPKK

SEQ ID NO: 81 - protein, 1345 aa, synthetic construct
MKVSKIKHARNAICKRKDSLSIGGRLYDVPNKKNQEQSIENYINNAIEGTKKFYTPFKKETVYFYAKKEDISAEKIQDEKVDELKSKIEKEITFRFEKTITRYLDQRNNYLNQTNEDELKRIIKNEWINASKNIKKENEKSEDILKQYFSDLFALESNGVSNQADFIYNFLTALFNMKLKRTISKEYVANGKHNNNVDTLTKGLMEFSNLAKGKKYNCNSKKFLYVLAAIREDYAKDEQAKSIAKSIKDNNVKVQVDKSQKDKLLTLSCAIKKEREYRNIDENLKMFAEQKNNVVLAVRDSIPVKNKITVSKDTLFKYVKDFVSECKTQNDKIGHMRCLIILFICGVEAYKKAVSCKQITDDFWGNILPVENVYFNSKIDEYLQEMFSLEGQYKTTSFLRKKIKDTVNNDIKNNFRTAIAEISGNEFDVYWMKYIQSTASKLLSNIDKLIKRNDQTYLCKAIYREWTSYLASKYIDMGKAVYNFTIPHNLSDLGNDIEFGKVLPEYKSGINSFHYEYIKAKENVHRSLAIASMYSVAVFSNNAVSEESRKIPKKEDVLAYSVNDFQKLGRENQVQRILQFFGGKSKWSSISETDYEFMFTLKRNLAVARNYSFHFAPSMSEVKEQYQKLALNMFNKEFSRINRLHMEKYYNNNVPVFYGKKSIINMLDFLYVNEVVIPEQIPSFNNVFPVKESNMLIHQWITSINSNGGYDLPVLTKFRQAFRFALKEIYYKGFLSQPDLYDKFIVALKAEKQEIEDNIAFVAEIEKKSAKSNDKLNAINNFSERVDEIKTKDFGMLCQQLMVDYEMQNQGRKSVFSTDRKEKAEKRGNKEKYQHFKMILWKMIRKAFKKYIDENCDIFGFLKEPSYKENYISKVSIDEFADSWNTPIKMAVTEVIKNNVGIRPWYFVAHFLHPKQLNHLKGAVKSYIQFVKDINKRAKSIDCSSISEEDMNIAYYEQLLLIFDFVSLFVGRVSNNIEDYFPSCQGKTSKEIYIEYINKYIEYDPTDENSVFFDEVNPIVNHAVVFSLMYGADKVLSSTDVHYMVSQKEYDTYIQLQKSKSMKNIFSKERCENIEEQKKLNEFQQLKNRVELHDLYSYTEMIIDHLAQLVSYAYMRERDLMYFQLGIQYIRLYHCKVVPEGKNKLRCLSNGSDINIKNGAILYQIIAMYNHDLKVFSCDENGNIIEDNKNTGDGVGMFLSKNGYGGYLEYINALQLFAINYDGYNGDNEFDRMHKLRNSIAHMKYFVGEGRSIIDYYTEIYNGFFRYSSSLRKSIGFIFDNIMMRYFIVTNISLDNIEKGKKRKVDIDSMVSDKFTYKYATGEEKLPVHNQQYMDIARNILYYKT

SEQ ID NO: 82 - protein, 1330 aa, synthetic construct
MKFSKESHRKTGVGITENDGAIGLLYRDPMKEEESIEEVVESRVDETTSLFNIFTVSQDIKKIIKQKDVESASIEDKEKAILMEEAKNVADLVIVVVRTLKSNNNLQTDLFVEKIKKQKKTEVKYTERGIDEALNRFLKKSFRVLPTKNALRELLIYTLIKNQSLSQEQKEKIQKNFIDKLVSEYNKTTVKQNVPKALRNQNMVIQPNPNKEGKILAISETPKSKETKTSEKDAFRDFLSEYAVLDENKRHELRVKLRRLVVLYFYGPDRVVFCDFDEWKDHESKKSEDVFFADIIKDRKVTKNADEKETVNVDATFDSIRTKNISSYRESLSFVNSHKEGFFDNQVLNKFWIHHIENEVERIYKHIKPSTPDYKFQIGYLSEKVWKGIINYLSIKYIAEGKAVYNYAMTALSNENNSKAFGKIDENYIKGISSFEYERIKAGETLQRECAVNIAFAARHLANATVNLDDVNTDFLLLKLDEPSEKEKQRGIKSLVDCAKPNVRKNLLQFFGGESIWKDFDFRDIEDIKLLDEFKKMIYSLRNASFHYKTENIDTGSRDTELIAEMFKKDCQNASGIQKNKMFSNNVVKFYREEDIQRLLEKLYSKINNRASQVPSFNAVFVRKNFKEYLAKHTNAYDLISDDLKNIWESAVYYLYKEIYYNLFLQDSNSFQYLKDYVDGLDEDKNCTDKKMLRAHIDFKNAFKEYAQMGNLSSVCQMIMTEYNNQNQGHRKAISGRSGMGKELKYKHYKIILLNGLRSAFSKYLKENEKDYGFINKPTVNILNLEDFLPTYTSNQYNDLVECVRSDFELQKWYVVGRLLNLKQVNQLIGSFRSYTQYVNDVKRRAELTGNPIRNMDVSVDVENIVGVLDVCTKLNGMTSNILEDYFDNKDDYARFVAMFVDFGLEEDSDYADAELAAFCNQEINGQKIGIYHDGTNPIINRNVILCKLYGATEIISKSEVKKVDLSTIREYFKLANKIKQYKVSGVCRDKVEQLNVREYQEIKNRVELRNIVEYSEIINELQGQLINWGYLRERDLMYFQLGFHYLSLHNNEEKPNGYDNAGDEYTGAILYQIVAMYTNGIDLIDVNGKSKGKENLPTGAKVNLFGSYSKEMLGLAKKEKDPIYIAGLELFEYVKEHNQCVDLRNYIEHFHYYAKHNRSMLDIYSEVFDRFFTYDMKYAKNVPNMMYNILLQHLVVAMFDFTSGEKKYGDSYDLLKKRASISIRKPNGLASEKFIYNLENGKKVIKLTARGRAYLEDIATIIYYPDDVPEEIIKDPIEDDKDAKGKDKKVKIQNDGKKRTFNNHNKKNNHGYRSKQPPKKKDYYSKRNH

SEQ ID NO: 83 - protein, 1400 aa, synthetic construct
MKIAKIRNSRSGISVAENKNEEQHSRQSFLYYAPGKDGKVRLESKDLEQHVMDCNRKAQNLYKVITPLEADFSRNKDDKEVFEDLAENFNAAINAALKSARTVKSNSESDLEIKLKQRVKKETEFILNLGENKIKMKKQEMQIQEMISELVLRRLKKTLRKEVVIADGRRMQMTEIVNSLMCAICLYGNVESFDPVVLNAFFEKVDSDYMKYKQIKQIVKSIESQSVKVKVTEENGKYLLIPANAEHKKKKYVFEFMKKYAVAEEKEKRELTGHIQELIGLYFCGESGYQKAGNSFADMIENHDCISEEIKEVLDSVETLENLSKQKREEKKEIREELERNKAAGKENKKGGKKEDKKSQNDELKETIKKLDSEIQKLSSEIRTKREELKNTTERVMSVHYRAAVEYLADKVGETILGQIKEKKVCACADLYWIDYIDDTVKKLLVDSKNKGNYRYTTGFLANHIWKEWTQYISGKYIELGKGVYHFAVPDLSGAVDGETVSICEVKPKYRSGISGFDYERIKAEESLEREMEEYILFAVNNFARAVAPEEEREREGHEDVLLMKTLSEIEEGSNTQSKNKQDKKNNSKKAIVLYADADRRILQFFGGQSRFREKEDSLINLYSGEDLYKEIRNELYAIRNVAFHYTTKADKDKVRKHEMAESFFEEELADVSEVFRKKYYSNNVWKFYSTEVINSLMEKIYSGRKYRAAQIPAFNKIISRPALPQIMERFINGTPLKRIKNNPDKNVVIQYWSALFFVLKELYYYDFLQEQENLEDNIKERFFRALERKSGQAEEDKKKAWESFRNRIEEIGKTSSFGEICQGLMVEYMLQNNDSSMVRKKNIYKKTSGRDTQIYKHYRTLLYICIRDAFTEYLKEKWPELRKPILLTKEWSEEEFCQTDGMKHLSLYDHLKEIFYSTVSGPSWYIAAHFINPKYLNHLIGSIRNYLQFTEDIERRAVSLGDSTDIEIAEKNLRYHNTLEVLEFVIQFCGKTTKVMEDYFESNQAYAAYLSDFVDYKTSKHVSDIETALHIFCEQKFKIDGKECMTGIYYDGGNLIPNRNIIRANMYGNVSCLKNSMKKITVAEIRQMYTDKAKLDTVLKEGVCRTRQEQEKLREFQNEKNRIELLDLAIYTEILNDMQAQLIGWSYLRERDLMYYQLGYYYTKLFWTDAIGKEDVRRKLVGDEVHIEDGAILYQILAYNSYDLPIISNIGEAAYFMKSEESIGGKAIGAFCKNFINAVSIYLEGLELFENTDEHDNIIKTRNYIEHFKYFIRSDRSMIDLYSEVYDRFFRHDYKLKKSVPTVLINILARNFMDAYIYMELGCKTVGTKKTQEHKATQIAFTDKGIESTALTYTIQPDPISKDNEKKEEKKKEEIPAHSEIFLHQFRSILEYRKGNGNN

SEQ ID NO: 84 - protein, 1024 aa, synthetic construct
NDAVIEGDFDEWQDHAKRKSDSGVFVEKVVLTKKDRNGKTIESLNVDGTKDAIRKKNISCYRESVAFAKEKKELFFEDDVLNKFWIHHIESEVERIYRSINNTTGNYKFELGYLSEKVWKGIINYLSIKYIAEGKAVYHYCMNALSDENKAFGVLDERYKNGITSFEYERIKAEETLQRECAVSISFATNHLANATVNLDEKNSDFLLIKNLEDVKKPHVVKNILQFFGGASTWEDFDFSGYDEVEVLAGFKEAIYSLRNTTFHFDTKNKDQVKWNTEFIGAMFAHDCKMARKVQKDKMYSNNVSDFYSEENIKKVLETLYSDIHERASQVPSFNKVFVRKNFPVYLTEQKLTPKFESAEMQKWQSAVYYLYKEIYYNAFLQDKKSYELLKNFVHKLPENEKDDKGKRTQESAANANFKEAFSEYEKEASSLSAICQLILTEYNNQNKGNRKAISARTNKADELIFQHYKMILLKGLEAAFTEYVKQNTVYDFIKNPTVLQNVPSQDDFLADYKTLQFEGLSKEVNATPDLQKWYMVGRLLNPKQLNQLIGSFRSYEQYVNDVARRAKQTKNPILDSEFSVDVHGILSVLSVCTSLSGVVSNNWKDYFDSEDAYAEYIAKFVDFGLKDNSDLANAALAQFCKKIGIYHDGTNPIVNRNVILCKLFGSSDILSKAGILKVDETVIKKYKTQEKETTGYRTTGICKDKAEQEKLKKFQELKNLVELRNIVEYLEILNELQGQLINWSYLRERDLMYFQLGFHYLCLQKNGKDISGYNKAGDVTGAILYQIVAMYTNGIPMIDKDGKSDDKGELQTSGKLNRFASYSETVNGDKWNIYLSGLELFENIDEHDQCVKLRNDIDHFHYFSEQNRSMLDYYSEVFDRFFTYDMKYTKNVANMLYNILLSHLVVATFGFGKGEKEVGEGKNKATKPRASIVLKTNGLASEKFTYKFSDGKTISLCARSNDFLKDVAALLYYPDKAPADVCRDTTVEDNRAKPENSKRDVNNFNRGNGSKNHSDKLRGKYED

SEQ ID NO: 85 - protein, 1333 aa, synthetic construct
MKISKESHRKTGVGIDDFGIKGLMYIDSLGKNDKKDSIATIVKRRADSTKRLYKLFGTKNSNVSKEAKYVAEIVNDSLKKIKLYPREYFDGKKKKERKDSTLRAGTLIKAIREEIKAKESTIKEKCKGEVVNNPLNISKNDIEQAVDSLLKKSFREKNEKDAIKLVLYSAFSIERKRLSDKELENITKYFINKLVDDYNKTILQKEIPIAIENQNMVVQPRIEDGTLVVAEIEKSSNRKKAEKAAFREFLSDYASLNENDRNEMLRKVRRLVDLYFYGGDFVRKDDFNVWSIHEERKKCKEKFIEQKNNNKNDEKPSKAGNIDEIKDEIRTENLHRYMDSLKAIKEDNSCYFENDILNKFWIHHIENEVERIYGHLKNDTGDYKFQLGYLSEKVWKGIINYLSIKYIAEGKAVYHYAMGALASDEGAKIFGRLDERYLNGISSFEYERIKAEETLQRECAVNVAFAANHLANATVQLDDINTDFLLLKNNELSEDNKKKGIKSLRDCATAKPNTLRNILQFFGGMSAWDDFDFGGREAIELLNDMKEMIYSLRNMSFHFKTENVNEGKWNTELIGRMFAHDCEVASLVQKNKLYSNNVAMFYGSSDLAKLLDKLYATYNERASQVPSFNTVFVRKNFPEYLKEQKITPTFSSDDTLKWQSAVYYLYKEIYYNAFLQDKESFNLLKRYVEKLDEHCQDKNQAGANRDFKEAFGIYSKSGSLSATCQMIMTEYNNQNKGNRVGISGRTNSDDKLIFQHYKMILFDGLRAAFFEYLKKNGEVYDFIKTPKLVTTLVDADVFLGDYKSHQYDSLVENIKTKPELQKWYITGRLLNPKQVNQLIGTFRSYVQYVNDVMRRAKQVGNPLTDATISIDVDNIIKVLDICTKLNGVTSNKIDDYFDSYDEYAKYLSKFVDFGLRGDKDLANAQLAAFCEREVEGQKIGIYHDGTNPVLNRNVVLCKLYGASDVVAQAGILPVDFEIIKKYMQLSGKIKEYQLTGICKTKQEQLKLKKYQELKNMVELRNIVDYAEIIDELQGQLINWGYLRERDLMYFQLGFHYLSLNNTAPKPEGYEMAGDYNGAILYQIVAMYTNGLSLIDADGKRKRNAKASTRFKIESFCSYSKSLRNVPGDTKEEIDPIYHAGLELFENIKEHDQCVYLRNYIEHFHYYAKMDRSLLDIYSEVFDRFFTYDMKYAKNVPNMLYNILLQHLVVPSFEFHSGEKKLGDHNSLSKKRATISLKSENGLASEKFTYKIGDKKNPIKLSARSNDYLSDVAAILYYPDTAPAQVVRDTVKEDNSAKEESKKSKDFAELPNNKNNNYHGRNNGYKNQKRDYSK

SEQ ID NO: 86 - protein, 1267 aa, synthetic construct
MKISKVNHHKSAVSLNENDIKGILYVDPHISKNTELEKYIAERISASQNLFSLFKNVKNKDIKKMCSKIDKTIKNKNGNVTASDLININVKKNDADGSEENIENLKKNIRKCVNVNTRDFIARDKRLKKALEKLLLNMRNIEKIDNGDEDIKRLALAINKAYLKDASGIKKSIENQNLSVQLSKDNEHFEVYPVTNLQNTNSVKKNEKEAFKHFMSDYARIDKHKRDELLLKLRRLNVLYFYGYDEAKNSLKYPEDIFADHEKRKCDKVKFVDKNILNEELLRLHNASIESIEKDNSLFFDDINLNRFWIRFIGNYTQKQFKKFLDSEDDFKKNRGYICEKAWKEILCFLSGKYIAEGKMVYQFAMDKIASNESEYDFGIIPERYKDGIKSFDFEMISANESIKRDIAVSVTFAVNNIARSVIDDKKAKNNYDLLSIDDEKLVSIMYDNEALKRNIYQFWGGMSKWKSFEDNNVFCSTKDYYHNLVIDIRNILANVRNENFHFKTSRYNAKKIDIDRLSKIFEFEANNASKVYRDKFYSNNLWEFYSDADMENALNILYSSYNYKASQIPAFGNVISRGKLNDFLVNDIKMTINYRDSETTERCRNAIYYLLKEVYYRGFLASNDVYEYFVNAIKKVEDEKKNFNFKDKQFQSIDDYVKRINELENNINTSNGKDKIAKICQFIMTENNQQNAGRKKVDTSKTKKNNKRIFEHYKMLLYKTLKYAYLDYLNKSAFAFIKNPKNDQMRRKDIAGTEFLPNWNAKTYISLVEKFKTENELQKMYIVAKLLPPRQLNRLIGEFKNYKSYTEDIERRANSNANILLVNKLYTDAVYNDIIQVLEMCMVQSGVITNKINDYFKDENAYSDYLRRYIDKDIENFELYKDGKNFILNRNVVLSKIYGAHEVLENTIEKISQKDIEDYKNALINVEEFRKTGKCESSEEQKNVNIYQRIKNRVTLANVLTYSEIINSLLGQLVGWSFLRERDLMYFQLGFHYECLQNDEEKMPTYNMIEVMQSDNTVLKINNAILYQIAAMYINGMPIYACDNNKEFSTVSTGLKIKMFCEYTLNAFNDKSRKEDYYNAGLEIFEDVKEHDNIANLRNHIDHFKYYSIRKDMPNEYAKSLLDLYSETFDRFFTYDNKLRKSVPVVFENILRRYFVILSLKFDTAKKNIGKNNEKDMAHFTVTPRSDSFTYKFKEKNPNDKDNNDNVRDEQFLVNIAKILYYPNEVPQDVLENINKKDNNKNNKKNNKNKTKSKDFTYKPFKDIKL

SEQ ID NO: 87 - protein, 1321 aa, synthetic construct
MKISKVDHNKTAVGNMDNMPIKGFIYKDPKRVENGTSVDINKRIKDARRTFESIYSVFDEKKFNKDTQDFNLVKQFNKDIRNVLKNSKGNIGFNELINGLNKFDSIKYEPDNIIYSGLKQSFQTEKIIKSIRILIRYVCENIKDINTDEKKLIENSFIGKIIKDYQKFDKCVHIPKAISNQNMVVQIDNDTRKLNLAKIPPHPESKHDDNSREREAFKRFLSGYSNLDDSVRHNIRVRLRRLVDLYFYGIEAVKLEDFNEWKDHEEKKARNDLFVDKQEITVKKESEGKKTEVDADLIKDTVRKKNIIRYRNTIKIASDNPTIFFDDQDVNVFWIHFIEKEVEKLFSKIKNNNDFVLKIGYVSEKVWKSMINYISIKYIALGKAVYHCALKDLDITDTSNLNIDLTKIDDKYLNGISSFHYELIKAEEDLQRDVSESVAFAANHLAHATISNINSKDDFLTLTYEDLKKKAYPNTMKNIMQFFGGYSKWKSFFEKNHSEDYDDIVFLRDIRNIIYSLRNSSFHFTTKKIDNDSWNTELIGNMFEKDCEIESTVLKNKLYFNNLPMYYADKPLENVLHRLYDSCHERASQVPSFNKVFIRKNFASILLKYNYNVVFNDDLDMLNWQSAIFYLFKEIYYNDFLQNDSSLSLFLRYVDDLKIEKDDRGKTTKSTRPNEDFKNAINFFKNDNTTLSSLCQQIMTEYNRQNDTGVKKRNYNVEKKHPKIFQHYRMILFDGLRHSFMRYVQENEKIFGFIKKPNKKSEGMSSVTEFLPNYFSRRYINLIETVKTNPELQKWYILGRLLNPRQVNLFIGDIRRYVQYINDILRRAKENGNNINNYIMPIDYKHIIEILEICTKLNGVVSNVVTDYFTDEDDYSSHIGKYIDFELENSAVTLRDFCNQIYKGKRIGIYYDGVNPIVNRNVVLCKLFGITDILDKNKKIKKVTKDVLYKYLDQQDKLVKYRKDGLCTNIAEQEFVNQYQWLKNRIELRDIVEYTEILNELHGQLIKWVYLRERDLMYFQLGFHYLCLNNNSPKPKEYVEINVGGVSLYGAILYQIISMYTNGMNLYYIYEGSDYISKMAVITGHRTGKKLRYHNIELNTYEINKIRDEKFNGTESIGSKIKYFLRYGDGIGHDKGYGEQLYYTGLEIFENLSEHDNIANLRKYIEHFRYFSQMDRSMLDIYSEVFDRFFSYDSKLRKNVINMMYNILLSHQVISKYQFGTSEKMIGTGEKSLKQQAACFMFKEKNGIDSDKFTYKYKQDGQNANKKTPQMLHARNTEFLMNVANLISYPKEVANDVVGCERENKKVRINNKVKNTAPVNN

SEQ ID NO: 88 - protein, 1202 aa, synthetic construct
MKVTKVDGISHKKYIEEGKLVKSTSEENRTDERFNDIEKKLSELLSIRLDIYIKNPDNASEENRIRRENLKEFFSNKVLHLKDGILYLKDRRELKNAVQNKNYSEQDISEYDLKNKNSFSVLKKILLLKLNENVKEEKIKFNSEELKEVFRKDIKKKLNKINSLKYSFEKNKANYQKINENNIEKVEGKSKRNIIYDYYRESAKRDAYVSNVQEAFDKLYKNSEEDIAKLFLEIENLTKHEKYKIREFYHNEIIGRKKENFAKIIYEEIQNVNKEENMKELIEKVGNPDMSELKKSQVFYKYYLDKEELNDENIKYVFCHFVEIEMSKLLKNYVYKKPSNISNDKVKRIFEYQSLKKLIENKLLNKLDTYVRNCGKYSFYLQDGEIATSDFIVGNRQNEAFLRNIIGVSSVAYFSLRNILETENEDFYEIENDITGRMRGKESNIVSGEVDKIYNENKQNEVKENLKMFYSYDFNMDNKNEIEDFFANIDEAISSIRHGIVHFNLDLDGKDIFAFKNIVPSEISKKMFQNEINEKKLKLKIFRQLNSANVFNYLEKYKILKYLKNTRFEFVNKNIPFVPSFTKLYNRIDDLKGKNSLKIYWKIPKDKEEKDAQIYLLKNIYYGEFLNKFVSNKNGNFFKISKEIIELNKNDKRNQKTGFYKLQKFENIEETIPKEYLAIIQSLYMINAGNEQDEEEKDTYIDFIQKIFLKGFMDYLNNNNRLKLIEINNDETNKILAEKKQEFDKILKKYEQNNRNLIEIPYEINEFVREIKLKEIGKILKYTERLNMFYLILKLLNHKELTNLKGSLEKYQSANKEETFSDQLELINLLNLDNNRVTIEDFELEADEIGKFLDFNSGNKVKDRKELKKFDTKENTGEYNKIYFDGENIIKHRAFYNIKKYGMLNLLEKISDEAKYKISIEELKKYSNKKNEIEDEKNHKMQENLHRKYARPRKDEKFTDEDYESYKQAIKNIEEYTHLKNKVEFNELNLLQGLLLRILHRLVGYTSIWERDLRFRLKGEFPENQYIEEIFNFDNKKNVKYKSGQIVEKYIKFYKELHQNDEVKINKYSSANIKVLKQEKKDLYIRNYIAHFNYIPHAEISLLEVLENLRKLLSYDRKLKNAVMKSVVDILKEYGFVVTFKIGAESNDKKIEIQSLKSEEIVHLKKLKLKDNDKKKEPIKTYRNSKELCKLVKVMFEYKMEN

SEQ ID NO: 89 - protein, 1201 aa, synthetic construct
MKVTKVDGISHKKYIEEGKLVKSTSEENRTSERFNDIEKKLSELLSIRLDIYIKNPDNASEENRIRRENLKEFFSNKVLHLKDGILYLKDRRELKNAVQNKNYSEQDISEYDLKNKNSFSVLKKILLLKLNENVKEEKIKFNSEELKEVFRKDIKKKLNKINSLKYSFEKNKANYQKINENNIEKVEGKSKRNIIYDYYRESAKRDAYVSNVKEAFDKLYKNSEEDIAKLFLEIENLTKHEKYKIREFYHNEIIGRKKENFAKIIYEEIQNVNKEENMKELIEKVGNPDMSELKKSQVFYKYYLDKEELNDENIKYVFCHFVEIEMSKLLKNYVYKKPSNISNDKVKRIFEYQSLKKLIENKLLNKLDTYVRNCGKYSFYLQDGEIATSDFIVGNRQNEAFLRNIIGVSSVAYFSLRNILETENEDFYEIENDITGRMRGKESNIVSGEVDKIYNENKQNEVKENLKMFYSYDFNMDNKNEIEDFFANIDEAISSIRHGIVHFNLDLDGKDIFAFKNIVPSEISKKMFQNEINEKKLKLKIFRQLNSANVFNYYEKDVIIKYLKNTKFNFVNKNIPFVPSFTKLYNKIDDLRGKNTLKFYWKIPKDKEEKDAQIYLLKNIYYGEFLNKFVKNKSKDFFKITDEVIKINKQDKRNQKTGYYKYQKFENIEKTVPVEYLAIIQSRDMINNQDKEEKNTYIDFIQQIFLKGFIDYLNKNNLKYIENNNNDIFSRIKIKKDSKERYDKILKNYEKNNRNLKEIPYEINEFVREIKLKEIGKILKYTERLNMFYLILKLLNHKELTNLKGSLEKYQSANKEETFSDQLELINLLNLDNNRVTIEDFELEADEIGKFLDFNSGNKVKDRKELKKFDTKENTGEYKKIYFDGENIIKHRAFYNIKKYGMLNLLEKISDEAKYKISIEELKNYSNKKNEIEDEKNHTMQENLHRKYARPRKDEKFTDEDYENYKRAIKNIEEYTHLKNKVEFNELNLLQGLLLRILHRLVGYTSIWERDLRFRLKGEFPENQYIEEIFNFDNKKNVKYKSGQIVEKYIKFYKELYQNDETKINKYSSANIKVLKQEKKDLYIRNYIAHFNYIPHAEISLLEVLENLRKLLSYDRKLKNAVMKSVVDILKEYGFVVTFKIGAESNDKKIEIQSLKSEEIVHLKKLKLKDNNKKKEPIKTYRNSKELCKLVKVMFEYKAEN

SEQ ID NO: 90 - protein, 1211 aa, synthetic construct
MKVTKVDGISHKKYIKEGKLVKSTSEENRTDERFNDIEKKLSELLSIRLDMYIKNPDNASEENRIRRENLKEFFSNKVLHLKDGILYLKDRRELKNAVQNKNYSEQDISEYDLKNKNSFSVLKKILLLKLNENVKEEKIKFNSEELKEVFRKDIKKKLNKINSLKYSFEKNKANYQKINENNIEKVEGKSKRNIIYDYYRESAKRDAYVSNVQEAFDKLYKNSEEDIAKLFLEIENLTKHEKYKIREFYHNEIIGRKKENFAKIIYEEIQNVNKEENMKELIEKVGNPDMSELKKSQVFYKYYLDKEELNDENIKYVFCHFVEIEMSKLLKNYVYKKPSNISNDKVKRIFEYQSLKKLIENKLLNKLDTYVRNCGKYSFYLQDGEIATSDFIVGNRQNEAFLRNIIGVSSVAYFSLRNILETENEDFYEIENDITGRMRGKESNIVSGEVDKIYNENKQNEVKENLKMFYSYDFNMDNKNEIEDFFANIDEAISSIRHGIVHFNLDLDGKDIFAFKNIVPSEISKKMFQNEINEKKLKLKIFRQLNSANVFRYLEKYKILNYLKRTRFEFVNKNIPFVPSFTKLYSRIDDLKGKNSLGIYWKTPKTNDDNKTKEITDAQIYLLKNIYYGEFLNYFMSNKNGNFFEISKEIIELNKNDKRNLKTGFYKLQKFEDIQEKIPKEYLANIQSLYMINAGNEQDEEEKDTYIDFIQKIFLKGFMTYLANNGRLSLIYIGSDETTSLAEKKQEFDKFLKKYEQNNNLIEIPYEINEFVREIKLKEIGKILKYTERLNMFYLILKLLNHKELTNLKGSLEKYQSANKEETFSDQLELINLLNLDNNRVTIEDFELEADEIGKFLDFNSGNKVKDRKELKKFDTKENTGEYNKIYFDGENIIKHRAFYNIKKYGMLNLLEKISDEAKYKISIEELKKYSNKKNEIEDEKNHKMQENLHRKYARPRKDEKFTDEDYESYKQAIKNIEEYTHLKNKVEFNELNLLQGLLLRILHRLVGYTSIWERDLRFRLKGEFPENQYIEEIFNFENKKNVKYKSGQIVEKYIKFYKELHQNDEVKINKYSSANIKVLKQEKKDLYIRNYIAHFNYIPHAEISLLEVLENLRKLLSYDRKLKNAVMKSVVDILKEYGFVVTFKIGAESNDKKIEIQSLKSEEIVHLKKLKLKDNDKKKEPIKTYRNSKELCKLVKVMFEYKMEEKKSEN

SEQ ID NO: 91 - protein, 1218 aa, synthetic construct
MKITKIDGISHKKYIKEGKLVKSTSEENKTDERFNDIEKKLSELLSIRLDTYIKNPDNASEENRIRRENLKEFFSNKVLHLKDGILYLKDRRELKNAVQNKNYSEQDISEYDLKNKNSFSVLKKILLLKLNEDIKEEKIKFNSEELKEVFRKDIEKKLDKINSLKYSLEENKANYQKINENNIKKVEGKSKRNIFYNYYKDSAKRNDYINNIQEAFDKLYKNSKEDIENLFFLIENSKKHEKYKIRECYHNKIIGRKKENFATIIYEEIQNVNKEENMKELIEKVGNPNVSELKKSQVFYKYYLNKEKLNDENIKYVFCHFVEIEMSKLLKNYVYKKPSNISNDKVKRIFEYQSLKKLIENKLLNKLDTYVRNCGKYSFYLQDGEIATSDFIVGNRQNEAFLRNIIGVSSVAYFSLRNILETENEDFYEIENDITGRMRGKESNIIKNNKGEISGEVDKIYNENKQNEVKNNLKMFYGYDFNMDNKNEIEDFFANIDEAISSIRHGIVHFNLDLDGKDIFAFKNIVPSEISKKMFQNEINEKKLKLKIFRQLNSANVFRYLEKYKILNYLKRTRFEFVNKNIPFVPSFTKLYSRIDDLKGKNSLGIYWKTPKTNDDNKTKEITDAQIYLLKNIYYGEFLNYFMSNKNGNFFEISKEIIELNKNDKRNLKTGFYKLQKFEDIQEKIPKEYLANIQSLYMINAGNEQDEEEKDTYIDFIQKIFLKGFMTYLANNGRLSLIYIGSDETTSLAEKKQEFDKFLKKYEQNNNLIEIPYEINEFVREIKLKEIGKILKYTERLNMFYLILKLLNHKELTNLKGSLEKYQSANKEETFSDQLELINLLNLDNNRVTIEDFELEADEIGKFLDFNSGNKVKDRKELKKFDTKENTGEYNKIYFDGENIIKHRAFYNIKKYGMLNLLEKISDEAKYKISIEELKKYSNKKNEIEDEKNHKMQENLHRKYARPRKDEKFTDEDYESYKQAIKNIEEYTHLKNKVEFNELNLLQSLLLRILHRLVGYTSIWERDLRFRLKGEFPENQYIEEIFNFENKKNVKYKSGQIVEKYIKFYKELHQNDEVKINKYSSANIKVLKQEKKDLYIRNYIAHFNYIPHAEISLLEVLENLRKLLSYDRKLKNAVMKSVVDILKEYGFVVTFKIGAESNDKKIEIQSLKSEEIVHLKKLKLKDNDKKKEPIKTYRNSKELCKLVKVMFEYKMEEKKSEN

SEQ ID NO: 92 - protein, 1212 aa, synthetic construct
MKVTKVGGISHKKYTSEGRLVKSESEENRTDERFNDIEKKLSALLNMRLDMYIKNPSNTEKENQKRIGKLKKFFSNKMVYLKDNTLSLKNGKKLENISETDISEYDVRDKKNFAVLKKIYLLKLNENVKEEKIKFNSEELKEVFRKDIKKKLNKINSLKYSFEKNKANYQKINENNIEKVEGKSKRNIIYDYYRESAKRDAYVSNVKEAFDKLYKNSEEDIAKLVLEIENLTKLEKYKIREFYHNEIIGRKKENFAKIIYEEIQNVNKEENMKELIEKVGNPDMSELKKSQVFYKYYLDKEELNDENIKYVFCHFVEIEMSKLLKNYVYKKPSNISNDKVKRIFEYQSLKKLIENKLLNKLDTYVRNCGKYNYYLQDGEIATSDFIARNRQNEAFLRNIIGVSSVAYFSLRNILETENEDFYEIENDITGRMRGKESNIIKNNKGEVSGEVDKIYNENKQNEVKENLKMFYSYDFNMDNKNEIEDFFANIDEAISSIRHGIVHFNLDLDGKDIFAFKNIVPSEISKKMFQNEINEKKLKLKIFRQLNSANVFRYLEKYKILNYLKRTRFEFVNKNIPFVPSFTKLYSRIDDLKGKNSLGIYWKTPKTNDDNKTKEITDAQIYLLKNIYYGEFLNYFMSNKNGNFFEISKEIIELNKNDKRNLKTGFYKLQKFEDIQEKIPKEYLANIQSLYMINAGNEQDEEEKDTYIDFIQKIFLKGFMTYLANNGRLSLIYIGSDETTSLAEKKQEFDKFLKKYEQNNNLIEIPYEINEFLREIKLKEIGKILKYTERLNMFYLILKLLNHKELTNLKGSLEKYQSANKEETFSDQLELINLLNLDNNRVTIEDFELEADEIGKFLDFNSGNKVKDRKELKKFDTKENTGEYNKIYFDGENIIKHRAFYNIKKYGMLNLLEKIADKAGYKISIEELKKYSNKKNEIEDEKNHKMQENLHRKYARPRKDEKFTDEDYESYKQAIKNIEEYTHLKNKVEFNELNLLQGLLLRILHRLVGYTSIWERDLRFRLKGEFPENQYIEEIFNFENKKNVKYKSGQIVEKYIKFYKELHQNDEVKINKYSSANIKVLKQEKKDLYIRNYIAHFNYIPHAEISLLEVLENLRKLLSYDRKLKNAVMKSVVDILKEYGFVVTFKIGAESNDKKIEIQSLKSEEIVHLKKLKLKDNDKKKEPIKTYRNSKELCKLVKVMFEYKMEEKKSEN

SEQ ID NO: 93 - protein, 1177 aa, synthetic construct
MENQTKTGKGIYYYHNSIEDKHYFGGFLNLAQNNIDNVIEEFRIRLNLPAYSIKNPVKKLSAIDIISNYFGQQSISYTDWERGVNILKEYLPVIDYLDLPVTDKEFENIDLKAKEKAKRDYFRKNLLGLLGTINNLRNFYTHHYHPLITIDENIAQFLDKTLFDVCMEVRKQKMKKDKTKQALKDGIAEELKKLIELKKAELKEKKIKTWNVEKNVEGAVYNDAFAHMIYKNKAEVTVLKDVHKTKVPTEKISGDLKVDFSMSSLIFLLSMFLSKKEIEQFKSNIERFKVKVIGESEIQEISKFNNSLKFMATHWVFSYLAFKGLKHRIKSVFDKETLLMQMIDELNKVPDEVYQTLSKEQQKEFLEDINEYVQDNEENRKSLEGSTIVQPLIRKRYDDKFNYFAIRFLDEFANFPTLKFFVTVGNFLHDKKNKQIKGSTVTSERLIKEKINVFGKLSEIVKYKSDYFSKKDINDETGWELYPNPSYLLNGNNIPVYIDLMGKGEDAKKCQIEINQLRKKTNPEKPRNLRASKEEIINIIYSKNKNIKHGEPTALLSANELPALLYELLINKKTGEDIENIIVNKIVQQFQTVSKYDNSINLPNSKITKKLKKADSAETIINTDKIIHAIKKEIEISDNKLELIRNNRNDYYKKSRKYIFYLKELGQEATWLAYDIKRFMPENARKNWKGIYHNELQKYLAFYDRNKNEAINLLSEFWDLNNDHFIGQELKNAFHEKYFDRFYESYLNARKNILTGFLELIKNLKNEPVILKKGLKEVFRIFTKRLYVINPMNIQKEQLLSKPICLPRGIFDEKPTFINDTKFEDNPSLFADWYQYTLKDTHQFQEFYNIQRDYKELFEKYKAVDNEFIKNKKKLNSEDMFSLFKYKEDMKIRRIKAQDLFIKLMVDELFNVIFSQKINLNLKEFYQTSDERYTNRLVAIEQSKRKKGDNSENKLNENFIWNKTVAVSLFNGQIYEPAVKLKDIGKFRKLENDEKVIQLLEYDKSKVWNKLGIEDEIENSPDSYERIRREKLLKEIQEFETYILKKENFDGVNHPEKFEQKQKTEDGKGSPNFKMYMVHGVLNKINALNNDDYKLLSKKEIDNIPVSEIMQLPVLVQKAYLLIIIRNKFVHNQFPDTHIFNLAKQFYPKINEETYAKYYFDVCTNIILEFKNQYNVL

SEQ ID NO: 94 - protein, 1199 aa, synthetic construct
MKVTKIDGLSHKKFEDEGKLVKIEDASQKNETLERLENLKGIKLGSYIKNPDKTKNKDNKKRRKGLKEYFSEITLRKENEKYVLLKGKKLKKINNDIKDTDIKAKDKKEEVFDILKEILKLNLLANDAEEKIQFDSIKLKNVFGKDFAKKELQIKSIEESLEKNRADYRKEFIEIENHKYGNVKGKNKRSRIYGYYKKSENHKKFEDNIREAFEKLYTEENIKKLYSKIEEVLKKTHLKSIVREFYKNEIIGESEFSKKNGDGISILYNQIKDSIKKEENFIEFIENIGNLELKDLTKSQIFYKYFLENEELNDENIKFVFCYFVEIEVSDLLKGNVYKASKIYENKIKNIFEYDKLKKLVINKLENKLNNYVRNCGKYNYHMENGDIATSDINMRNRQTEAFLRSMIGVSSFGYFSLRNILGVNDDDFYEIEEDLTKDERKKESSTLKKVKEDIASKSIFEKVVDKSFQKKGIYNIKENLKMFYGDSFDDADEDELKQFFVNMLNGITSIRHRVVHYNMNTNSENIFNFSDIEVSRLLKNIFEKETDKRELKLKIFKQLNSAGVFDYWESWKIKKYLENIEFKFVNKNIPFVPSFTKLYSRIDDLKNSLGIYWKTPKTNDDNKTKEIIDAQIYLLKNIYYGEFLNYFMSNNGNFFEISKEIIELNKNDKRNLKTGFYKLQKFEDIQEKIPKEYLANIQSLYMINAGNQDEEEKDTYIDFIQKIFLKGFMTYLANNGRLSLIYIGSDEETNTSLAEKKQEFDKFLKKYEQNNNIKIPYEINEFLREIKLGNILKYTERLNMFYLILKLLNHKELTNLKGSLEKYQSANKEEAFSDQLELINLLNLDNNRVTEDFELEADEIGKFLDFNGNKVKDNKELKKFDTNKIYFDGENIIKHRAFYNIKKYGMLNLLEKIADKAGYKISIEELKKYSNKKNEIEKNHKMQENLHRKYARPRKDEKFTDEDYESYKQAIENIEEYTHLKNKVEFNELNLLQGLLLRILHRLVGYTSIWERDLRFRLKGEFPENQYIEEIFNFENKKNVKYKGGQIVEKYIKFYKELHQNDEVKINKYSSANIKVLKQEKKDLYIRNYIAHFNYIPHAEISLLEVLENLRKLLSYDRKLKNAVMKSVVDILKEYGFVATFKIGADKKIGIQTLESEKIVHLKNLKKKKLMTDRNSEELCKLVKIMFEYKMEEKKSEN

SEQ ID NO: 95 - protein, 1145 aa, synthetic construct
MKVTKVGGISHKKYTSEGRLVKSESEENRTDERLSALLNMRLDMYIKNPSSTETKENQKRIGKLKKFFSNKMVYLKDNTLSLKNGKKENIDREYSETDILESDVRDKKNFAVLKKIYLNENVNSEELEVFRNDIKKKLNKINSLKYSFEKNKANYQKINENNIEKVEGKSKRNIIYDYYRESAKRDAYVSNVKEAFDKLYKEEDIAKLVLEIENLTKLEKYKIREFYHEIIGRKNDKENFAKIIYEEIQNVNNMKELIEKVPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQDGEIATSDFIARNRQNEAFLRNIIGVSSVAYFSLRNILETENENDITGRMRGKTVKNNKGEEKYVSGEVDKIYNENKKNEVKENLKMFYSYDFNMDNKNEIEDFFANIDEAISSIRHGIVHFNLELEGKDIFAFKNIAPSEISKKMFQNEINEKKLKLKIFRQLNSANVFRYLEKYKILNYLKRTRFEFVNKNIPFVPSFTKLYNRIDNLKGSNALNLGYINIPKRKEVRDSQIYLLKNIYYGEFVEEFVNNNDNFEKIFREIIEINKNAGTNTKTKFYKLEKFETLKANTPTEYLEKLQSLHQINYDREKIEEDKDIYVDFVQKIFLKGFINYLQESGSLKYLLNLEKNEVIANKKSSYDEKLKLWQNNGSDLSKMPEEIYEYIKKIKINKINYSDRMSIFYLLLKLIDYKELTNLRGNLEKYESMNKNKIYSEELNIVNLVSLDNNKVRANFELEAEDVRKFLKTETLIKDARQLNNFSERFSDGENVIKHRSFYNIKKYGILDLLEKIVAKADLKITKEEIKKYENLQKELEKNDFYKIQEKIHKKYNQKPNLILRTENKKDFNDYKKAIENIQNYTQLKNKIEFNDLNLLQSLLFRILHRLAGYTSLWERDLQFKLKGEFPEDKYIDEIFNSDRNNNQKYKSGGIAYKYVDFLIEKEEGKRAGKNKVKKRSEKEGSFIIRNYIAHFNYIPDAEKSILEMLEELRALLKYDRKLKNAVMKSVKDIFKEYGFIVEFGISHESNSKKIKVVNVESEKIKHLKNNGLVTKRNSEDLCNLVKVMLEYKKS

SEQ ID NO: 96 - protein, 1167 aa, synthetic construct
MKVTKVGGISHKKYTSEGRLVKSESEENRTDERLSALLNMRLDMYIKNPSSTETKENQKRIGKLKKFFSNKMVYLKDNTLSLKNGKKENIDREYSETDILESDVRDKKNFAVLKKIYLNENVNSEELEVFRNDIKKKLNKINSLKYSFEKNKANYQKINENNIEKVEGKSKRNIIYDYYRESAKRDAYVSNVKEAFDKLYKEEDIAKLVLEIENLTKLEKYKIREFYHEIIGRKNDKENFAKIIYEEIQNVNNMKELIEKVPDMSELKKSQVFYKYFLENEELNDENIKFVFCYFVEIEVSDLLKGNVYKASKIYENKIKNIFEYDKLKKLVINKLENKLNNYVRNCGKYNYHMENGDIATSDINMRNRQTEAFLRSMIGVSSFGYFSLRNILGVNDDDFYEIEEDLTKDERKKESSTLKKVKEDIASKSIFEKVVDKSFQKKGIYNIKENLKMFYGDSFDDADEDELKQFFVNMLNGITSIRHRVVHYNMNTNSENIFNFSDIEVSRLLKNIFEKETDKRELKLKIFKQLNSAGVFDYWESWKIKKYLENIEFKFVNKNIPFVPSFTKLYNRIDNLKGSNALNLGYINIPKRKEVRDSQIYLLKNIYYGEFVEEFVNNNDNFEKIFREIIEINKNAGTNTKTKFYKLEKFETLKANTPTEYLEKLQSLHQINYDREKIEEDKDIYVDFVQKIFLKGFINYLQESGSLKYLLNLEKNEVIANKKSSYDEKLKLWQNNGSDLSKMPEEIYEYIKKIKINKINYSDRMSIFYLLLKLIDYKELTNLRGNLEKYESMNKNKIYSEELNIVNLVSLDNNKVRANFELEAEDVRKFLKTETLIKDARQLNNFSERFSDGENVIKHRSFYNIKKYGILDLLEKIVAKADLKITKEEIKKYENLQKELEKNDFYKIQEKIHKKYNQKPNLILRTENKKDFNDYKKAIENIQNYTQLKNKIEFNDLNLLQSLLFRILHRLAGYTSLWERDLQFKLKGEFPENQYIEEIFNFENKKNVKYKGGQIVEKYIKFYKELHQNDEVKINKYSSANIKVLKQEKKDLYIRNYIAHFNYIPHAEISLLEVLENLRKLLSYDRKLKNAVMKSVVDILKEYGFVATFKIGADKKIGIQTLESEKIVHLKNLKKKKLMTDRNSEELCKLVKIMFEYKMEEKKSEN

SEQ ID NO: 97 - protein, 1237 aa, synthetic construct
MKVTKIDGISHKKYEEEGKLVKSKNIKEKNKTEERFNDIEKKLKELLEIKLDNYIKNPDNAKEEENKIRRENLKEYFSKIILHLEDEKLILKNKKKLKNIVQNKNYEEEDINEYDLKNKKSFNVLKKILKLKLNENVKEEKITFNSEELKEVFGKDLKKKLNQIKSIKNSLEKNKANYKKESIEIGNNNIEKVKGKNKRSIIYEYYRKSAKHEKYVNNIKEAFDKLYTNSEEDIKKLFLEIEKLTKKTHEKSKIREFYHNEIIGKNNFSKDKGDFAKILYEEIQNSINKEENFKELIEKVGNPELKELTKSQIFYKYYLDKEELNDENIKYVFCHFVEIEVSKLLKNYVYKKPSNINKDKIKNIFEYKKLKKLIENKLLNKLNTYVRNCGKYNYYLENGDIATSDIIMRNRQNEAFLRSIIGVSSFAYFSLRNILGTDNEDFYEIEEDITGKERGKESNVLKTAKENIGEEKIVEGEVDKIYEENKKNEIKENLKMFYGNSFDMDNENEIKEFFVNIDEAISSIRHGIVHFNMDTNSENIFNFNNIEVSKISKKIFENEINEKKLKLKIFRQLNSANVFDYLEKWKIKKYLKNIKFKFVNKNIPFVPSFTKLYNRIDDLKGSNSLKIGYWKIPKKKEAKDAQIYLLKNIYYGEFLDKFVNNKNGNFFKISKEIIEINKNAGKNKKTGFYKLEKFENIEETTPKEYLEIIQSLYMINYGKEKDEEEKDIYIDFIQKIFLKGFIDYLKKNNSLKLLELLNLKEKNEIIADKKSKYDKKLKKYEKNNRNLIEIPKEINEFIKKIKIAEINKILNYTDRLNIFYLLLKLLNHKELTNLKGNLEKYQSMNKNEIYSEELELINLVNLDNNKVKIENFELEAEEIGKFLETNSRGNKIKNIKELKNFDTIENTGEYNKIYADGENIIKHRSFYNIKKYGMLDLLEKISEKANYKISKEELKKYSNLKNELEDEKNDFYKIQENLHRKYNRKPKNILKKENKEDYENYKKAIENIEEYTHLKNKIEFNELNLLQSLLLRILHRLVGYTSIWERDLRFRLKGEFPEDQYIEEIFNFDNKKNKKYKSGQIAEKYIKFLKEKEEGKKAKILGYNSKKIKKLKEKEGNLFIRNYIAHFNYIPNAEISILEILEKLRELLSYDRKLKNAVMKSVKDILKEYGFIVEFKISAESNDKKIKILSVKSEEIVHLKKLKLKDNDKKKNGLKTYRNSEDLCKLVKVMFEYKKEN

SEQ ID NO: 98 - protein, 1301 aa, synthetic construct
MKVTKIDGISHKKYKEEGKLVKSTSIKKEEKSNNDTNKEEKNKTEERFNDIEKKLSELLSIKLDIYIKNPDNASNKDKDAEEENKIRRENLKEYFSNIVLHLEDGKLYLKNKKELKNIVQNKNYSEEDISEYDLKNKNSFNVLKKILLLKLNENVKEEKIKFNSEELKEVFRKDIKKKLNQIKSIKNSLEKNKANYKKESIEIGNNNIEKVKGKNKRSIIYEYYRESAKHEEYVSNIKEAFDKLYTNSEEDIAKLFLEIENLTKKTHEKYKIREFYHNEIIGKNCDNFSKDKEDFAKILYEEIQNSINKEENFKELIEKVGNPEMKELTKSQIFYKYYLDKEELNDENIKYVFCHFVEIEVSKLLKNYVYKKPSFNNINKDKIKNIFEYKKLKKLIENKLLNKLNTYVRNCGKYNYYLQNGDIAVTSDFIVGNRQNEAFLRSIIGVSSVAYFSLRNILETDNEDFYEIEEDITGKMRGKESNILKEDSILKTVIKNNKGETNEDKIVSGEVDKIYNENNKKNEVKENLKMFYGNDFDMDNKNEIKDFFVNIDEAISSIRHGIVHFNMDANGKNIFAFNNIELSEISKKIFQNEINEKKLKLKIFRQLNSANVFDYLEKYKIIKYLKNTKFNFVNKNIPFVPSFTKLYNRIDDLKGNNSLKIGNYWKIPKTNDDNKEKEAKDAQIYLLKNIYYGEFLDKFVNNKNGNFFKISKEIIELNKNDKRNKKTGFYKLEKFENIEETIPKEYLEIIQSLYMINYGKEKDEEEKDIYIDFIQKIFLKGFIDYLKKNNSLKLLELGNNNDNEETFSRNKIIADKKNKFDKKLKKYEKNNRNLIEIPKEINEFVRKIKIAEINKILNYTDRLNMFYLLLKLLNHKELTNLKGNLEKYQSMNKNEIYSEELELINLVNLDNNKVKIENFELEAEEIGKFLETNSIEEINRGNKIKNLKELKNFDTIENTGEYNKIYSDGENIIKHRSFYNIKKYGMLDLLEKISEKANYKISKEELKKYSNLKNELEDEKNDFYKIQENLHRKYARKRKDILKKNNDEDYENYKKAIENIEEYTHLKNKIEFNELNLLQSLLLRILHRLVGYTSIWERDLRFRLKGEFPEDQYIEEIFNFDNKKNVKYNEKNEKSGQIVEKYIKFLKELHENEEAKINKYNSAKIKKLKEIIEEKNLYIRNYIAHFNYIPNAEISILEILENLRELLSYDRKLKNAVMKSVKDILKEYGFVVEFKIGAESNDKKIEIQSVKSEEIVHLKKLKLKDNDKKKNPLKTYRNSEDLCKLVKVMFEYKMEEKKSEN

SEQ ID NO: 99 - protein, 1225 aa, synthetic construct
MKVTKIDGISHKKYKEEGKLVKSESEENRTDERLSALLNMRLDMYIKNPSSTETKENQKRIGKLKKFFSNIVLHLEDGKLYLKNKKELKNIVQNKNYSEEDISEYDLKNKNSFNVLKKILLLKLNENVKEEKIKFNSEELKEVFRKDIKKKLNQIKSIKNSLEKNKANYKKESIEIGNNNIEKVKGKNKRSIIYEYYRESAKHEEYVSNIKEAFDKLYTNSEEDIAKLFLEIENLTKKTHEKYKIREFYHNEIIGRKNDKEDFAKILYEEIQNSINKEENFKELIEKVGNPEMKELTKSQIFYKYYLDKEELNDENIKYVFCHFVEIEVSKLLKNYVYKKPSFNNINKDKIKNIFEYKKLKKLIENKLLNKLNTYVRNCGKYNYYLQNGDIAVTSDFIVGNRQNEAFLRSIIGVSSVAYFSLRNILETDNENDITGRMRGKTVKNNKGEEKYVSGEVDKIYNENNKKNEVKENLKMFYGNDFDMDNKNEIKDFFVNIDEAISSIRHGIVHFNMDANGKNIFAFNNIELSEISKKIFQNEINEKKLKLKIFRQLNSANVFDYLEKYKIIKYLKNTKFNFVNKNIPFVPSFTKLYNRIDDLKGNNSLKIGNYWKIPKTNDDNKEKEAKDAQIYLLKNIYYGEFLDKFVNNKNGNFFKISKEIIELNKNDKRNKKTGFYKLEKFENIEETIPKEYLEIIQSLYMINYGKEKDEEEKDIYIDFIQKIFLKGFIDYLNKNNLKYIESNNNNDNNDIFSKIKIKKDNKEKYDKILKKYEKNNRNLIEIPKEINEFVRKIKIAEINKILNYTDRLNMFYLLLKLLNHKELTNLKGNLEKYQSMNKNEIYSEELELINLVNLDNNKVKIENFELEAEEIGKFLDFNENKIKDRKELKKFDTNKIYSDGENIIKHRSFYNIKKYGMLDLLEKISEKANYKISKEELKKYSNLKNELEDEKNDFYKIQENLHRKYARKRKDILKKNNDEDYENYKKAIENIEEYTHLKNKIEFNELNLLQSLLLRILHRLVGYTSIWERDLRFRLKGEFPEDQYIEEIFNFDNKKNVKYNEKNEKSGQIVEKYIKFLKELHENEEAKINKYNSAKIKKLKEIIEEKNLYIRNYIAHFNYIPNAEISILEILENLRELLSYDRKLKNAVMKSVKDILKEYGFVVEFKIGAESNDKKIEIQSVKSEEIVHLKNLKKKKLMTDRNSEDLCKLVKVMFEYKMEEKKSEN

SEQ ID NO: 100-103 - skipped (4 entries)

SEQ ID NO: 104 - protein, 5 aa, synthetic construct
GSGGS

SEQ ID NO: 105 - protein, 6 aa, synthetic construct
GGSGGS

SEQ ID NO: 106 - protein, 4 aa, synthetic construct
GGGS

SEQ ID NO: 107 - protein, 4 aa, synthetic construct
GGSG

SEQ ID NO: 108 - protein, 5 aa, synthetic construct
GGSGG

SEQ ID NO: 109 - protein, 5 aa, synthetic construct
GSGSG

SEQ ID NO: 110 - protein, 5 aa, synthetic construct
GSGGG

SEQ ID NO: 111 - protein, 5 aa, synthetic construct
GGGSG

SEQ ID NO: 112 - protein, 5 aa, synthetic construct
GSSSG

SEQ ID NO: 113 - protein, 6 aa, synthetic construct
HHHHHH

SEQ ID NO: 114 - DNA, 20 bp, synthetic construct
  source 1..20: mol_type = other DNA
  misc_difference 1..20: note = This sequence may encompass 5-20 nucleotides
aaaaaaaaaaaaaaaaaaaa

SEQ ID NO: 115 - DNA, 20 bp, synthetic construct
  source 1..20: mol_type = other DNA
  misc_difference 1..20: note = This sequence may encompass 5-20 nucleotides
tttttttttttttttttttt

SEQ ID NO: 116 - DNA, 20 bp, synthetic construct
  source 1..20: mol_type = other DNA
  misc_difference 1..20: note = This sequence may encompass 5-20 nucleotides
cccccccccccccccccccc

SEQ ID NO: 117 - DNA, 20 bp, synthetic construct
  source 1..20: mol_type = other DNA
  misc_difference 1..20: note = This sequence may encompass 5-20 nucleotides
gggggggggggggggggggg

SEQ ID NO: 118 - DNA, 29 bp, synthetic construct
  source 1..29: mol_type = other DNA
  modified_base 1: mod_base = OTHER; note = Nucleotide attached to Horseradish peroxidase enzyme
  modified_base 29: mod_base = OTHER; note = Biotin triethylene glycol modified nucleotide
  misc_feature 1..12: note = DNA
  misc_feature 13..17: note = RNA
  misc_feature 18..29: note = DNA
ttttttttttttttttttttttttttttt

SEQ ID NO: 119 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFNLSTCILQAVEHQNLDELHPYYDWSKWYINDKSGKLKKSITNNHIQIGDGESKRQKALLMWETDFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAVRANLLRKGKYQHHDLKADATSENLTNLKRDEAFVMNLITTSAFAANNIRNIIDPEQIKDIVGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWNGRYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFLKQSAYQFINTPQPQINPTDTNQQQADKLKALEKKICPFCHIQNNKIDPAKDEQIAFYVYCKLLDANHLSTLRNELIKYRAASESELFIHLTEIIELCLLHADNVPADYWQIYKNKEQCLQRLQPFIAENSDYTNWGDLYVQTDDETPVVHSGIELSVKYGTASLLEKLIASDAKFKITENEFDLWHNARATIEAMIKKREELHNLWLEAKEKDDNEKKTRIREKNNYARRFVETKGEDYIDICDYIDRYNWLNNKLHFEHLKSLQNITIEILGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITPKIIFHLGTKKESDNIYTFQVPTAYCNMCKQLLEMKN

SEQ ID NO: 120 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTASLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITSKIIFHLGTKKESDNIYTFQVPTAYCNMCKQLLEMKN

SEQ ID NO: 121 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTAKLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 122 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAVRANLLRQGKYQHHDLKADATSENLTNLKRDEAFVMNLITTCAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTTVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDARHLSTLRNELIKYRASSESELFIHLTEIIELCLLHADNVPADYNQIYKNKEQCLQRLQPFIAENSDYTNWGDLYVQTDDETPVVHRGIELSVKYGTAKLLEKLIASDAKFKITENEFDLWHNARATIEAMIKKREEYHNLWLEAKEKDDKEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 123 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSKKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAVRANLLRQGKYQHHDLKADATSENLTNLKRDEAFVMNLITTCAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTTVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWNMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDARHLSTLRNELIKYRASSESELFIHLTEIIELCLLHADNVPADYNQIYKNKEQCLQRLQPFIAENSDYTNWGDLYVQTDDETPVVHRGIELSVKYGTAKLLEKLIAKDAKFKITENEFDLWHNARATIEAMIKKREEYHNLWLEAKEKDDKEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLRYDRKLKNAVAKTFIDLFDKHGMVLKLKFDSQMHQLQVGSITSKKIFHLGTKKESDNIYTFQVPTAYCNMCKQLLEMKN

SEQ ID NO: 124 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAVRANLLRQGKYQHHDLKADATSENLTNLKRDEAFVMNLITTCAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTTVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQQNMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDARHLSTLRNELIKYRASSESELFIHLTEIIELCLLHADNVPGDYNQIYKNKEQCLQRLQPFIAENSDYTNWGDLYVQTDRETPVVHRGIELSVKYGTAKLLEKLIASDAKFKITENEFDLWHNARATIEAMIKKREEYHNLWLEAKEKDDKEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 125 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFQLPVKYQKEGQWVQFNLSTLILQAVEHQNLDELHPYYDWSKWYINDKSGKLKKSITNNHIQIGDGESKRQKALLMWETDFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAVRANLLRKGKYQHHDLKADATSENLTNLKRDEAFVMNLITTSAFAANNIRNIIDPEQIKDIVGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWNGRYFLLKIIYNNLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRAASESELFIHLTEIIELCLLHADNVPADYWQIYKNKEQCLQRLQPFIAENSDYTNWGDLYVQTDDETPVVHSGIELSVKYGTASLLEKLIASDAKFKITENEFDLWHNARATIEAMIKKREELHNLWLEAKEKDDNEKKTRIREKNNYARRFVETKGEDYIDICDYIDRYNWLNNKLHFVHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITPKIIFHLGTKKESDNIYTFQVPTAYCNMCKQLLEMKN

SEQ ID NO: 126 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFNLSTCILQAVEHQNLDELHPYYDWSKWYINDKSGKLKKSITNNHIQIGDGESKRQKALLMWETDFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAVRANLLRKGKYQHHDLKADATSENLTNLKRDEAFVMNLITTSAFAANNIRNIIDPEQIKDIVGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSGRYFLLKIIYNYLFLPEFTNNDKVFADTVAWLLGKNRQQAQSSKNKNAYAFADIRLMESSESITDYMAYVESQWIMEENKKEDDKKEDTRINFEKFVLQLFVKGFDTFLKQSAYQFINTPQPQINPTDTNQQQADKLKALEKKICPFCHIQNNKIDPAKDEQIAFYVYCKLLDANHLSTLRNELIKYRAASESELFIHLTEIIELCLLHADNVPADYWQIYKNKEQCLQRLQPFIAENSDYTNWGDLYVQTDDETPVVHSGIELSVKYGTASLLEKLIASDAKFKITENEFDLWHNARATIEAMIKKREELHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITSKIIFHLGTKKESDNIYTFQVPTAYCNMCKQLLEMKN

SEQ ID NO: 127 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFNLSTCILQAVEHQNLDELHPYYDWSKWYINDKSGKLKKSITNNHIQIGDGESKRQKALLMWETDFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRKGKYQHHDLKADATSENLTNLKRDEAFVMNLITTSAFAANNIRNIIDPEQIKDIVGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSGRYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRAASESELFIHLTEIIELCLLHADNVPADYWQIYKNKEQCLQRLQPFIAENSDYTNWGDLYVQTDDETPVVHSGIELSVKYGTASLLEKLIASDAKFKITENEFDLWHNARATIEAMIKKREELHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITPKIIFHLGTKKESDNIYTFQVPTAYCNMCKQLLEMKN

SEQ ID NO: 128 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFNLSTCILQAVEHQNLDELHPYYDWSKWYINDKSGKLKKSITNNHIQIGDGESKRQKALLMWETDFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAVRANLLRKGKYQHHDLKADATSENLTNLKRDEAFVMNLITTSAFAANNIRNIIDPEQIKDIVGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWNGRYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFLKQSAYQFINTPQPQINPTDTNQQQADKLKALEKKICPFCHIQNNKIDPAKDEQIAFYVYCKLLDANHLSTLRNELIKYRAASESELFIHLTEIIELCLLHADNVPADYWQIYKNKEQCLQRLQPFIAENSDYTNWGDLYVQTDDETPVVHSGIELSVKYGTASLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEILGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITPKIIFHLGTKKESDNIYTFQVPTAYCNMCKQLLEMKN

SEQ ID NO: 129 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFQLPVKYQKEGQWVQFNLSTLILQAVEHQNLDELHPYYDWSKWYINDKSGKLKKSITNNHIQIGDGESKRQKALLMWETDFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAVRANLLRKGKYQHHDLKADATSENLTNLKRDEAFVMNLITTSAFAANNIRNIIDPEQIKDIVGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWNGRYFLLKIIYNNLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRAASESELFIHLTEIIELCLLHADNVPADYWQIYKNKEQCLQRLQPFIAENSDYTNWGDLYVQTDDETPVVHSGIELSVKYGTASLLEKLIASDAKFKITENEFDLWHNARATIEAMIKKREELHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITPKIIFHLGTKKESDNIYTFQVPTAYCNMCKQLLEMKN

SEQ ID NO: 130 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFQLPVKYQKEGQWVQFNLSTCILQAVEHQNLDELHPYYDWSKWYINDKSGKLKKSITNNHIQIGDGESKRQKALLMWETDFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAVRANLLRKGKYQHHDLKADATSENLTNLKRDEAFVMNLITTSAFAANNIRNIIDPEQIKDIVGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAYYGFDQLKFLLDVVKFHLCRTVVPFAPGFKKVMKGGTGYQNAKQDESFYNLELTAYQLKDDFSEEAWSGRYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEHIAFYVYCKLLDANHLSTLRNELIKYRAASESELFIHLTEIIELCLLHADNVPADYWQIYKNKEQCLQRLQPFIAENSDYTNWGDLYVQTDDETPVVHSGIELSVKYGTASLLEKLIASDAKFKITENEFDLWHNARATIEAMIKKREELHNLWLEAKEKDDNEKKTRIREKNNYARRFVETKGEDYIDICDYIDRYNWLNNKLHFVHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITPKIIFHLGTKKESDNIYTFQVPTAYCNMCKQLLEMKN

SEQ ID NO: 131 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFQLPVKYQKEGQWVQFELSTCILQAVEHQNLDELHPYYDWSKWYINDKSGKLKKSITNNHIQIGDGESKRQKALLMWETDFTDKGELDLETYHEKYIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAVRANLLRKGKYQHHDLKADATSENLTNLKRDEAFVMNLITTSAFAANNIRNIIDPEQIKDIVGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAYYGFDQLKFLLDVVKFHLCRTVVPFAPGFKKVMKGGTGYQNSKQDESFYNLELTAYQLKDDFSEEAWNGRYFLLKIIYNNLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRAASESELFIHLTEIIELCLLHADNVPADYWQIYKNKEQCLQRLQPFIAENSDYTNWGDLYVQTDDETPVVHSGIELSVKYGTASLLEKLIASDAKFKITENEFDLWHNARATIEAMIKKREELHNLWLEAKEKDDNEKKTRIREKNNYARRFVETKGEDYIDICDYIDRYNWLNNKLHFEHLKSLQNITIEILGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITPKIIFHLGTKKESDNIYTFQVPTAYCNMCKQLLEMKN

SEQ ID NO: 132 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELHPYYDWSKWYINDKSGKLKKSITNNHIQIGDGESKRQKALLMWETDFTDKGELDLETYHEKYIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRKGKYQHHDLKADATSENLTNLKRDEAFVMNLITTSAFAANNIRNIIDPEQIKDIVGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWNGRYFLLKIIYNYLFLPEFTNNDKVFADTVAWVLGKNRQQAQSSKNKNAYAFADIRLMESSESITDYMAYVQSQWIMEENKKEDDKKEDTRINFEKFVLQLFVKGFDTFLKQSAYQFINTPQPQINPTDTNQQQADKLKALEKKICPFCHIQNNKIDPAKDEQIAFYVYCKLLDANHLSTLRNELIKYRAASESELFIHLTEIIELCLLHADNVPADYWQIYKNKEQCLQRLQPFIAENSDYTNWGDLYVQTDDETPVVHSGIELSVKYGTASLLEKLIASDAKFKITENEFDLWHNARATIEAMIKKREELHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITPKIIFHLGTKKESDNIYTFQVPTAYCNMCKQLLEMKN

SEQ ID NO: 133 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFQLPVKYQKEGQWVQFELSTCILQAVEHQNLDELHPYYDWSKWYINDKSGKLKKSITNNHIQIGDGESKRQKALLMWETDFTDKGELDLETYHEKYIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRKGKYQHHDLKADATSENLTNLKRDEAFVMNLITTSAFAANNIRNIIDPEQIKDIVGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWNGRYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFLKQSAYQFINTPQPQINPTDTNQQQADKLKALEKKICPFCHIQNNKIDPAKDEQIAFYVYCKLLDANHLSTLRNELIKYRAASESELFIHLTEIIELCLLHADNVPADYWQIYKNKEQCLQRLQPFIAENSDYTNWGDLYVQTDDETPVVHSGIELSVKYGTASLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITPKIIFHLGTKKESDNIYTFQVPTAYCNMCKQLLEMKN

SEQ ID NO: 134 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELHPYYDWSKWYINDKSGKLKKSITNNHIQIGDGESKRQKALLMWETDFTDKGELDLETYHEKYIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAVRANLLRKGKYQHHDLKADATSENLTNLKRDEAFVMNLITTSAFAANNIRNIIDPEQIKDIVGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAYSGRYFLLKIIYNNLFLPEFTNNDKVFADTVAWLLGKNRQQAQSSKNKNAYAFADIRLMESSESITDYMAYVQSQWIMEENKKEDDKKEDTRINFEKFVLQLFVKGFDTFLKQSAYQFINTPQPQINPTDTNQQQADKLKALEKKICPFCHIQNNKIDPAKDEQIAFYVYCKLLDANHLSTLRNELIKYRAASESELFIHLTEIIELCLLHADNVPADYWQIYKNKEQCLQRLQPFIAENSDYTNWGDLYVQTDDETPVVHSGIELSVKYGTASLLEKLIASDAKFKITENEFDLWHNARATIEAMIKKREELHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITPKIIFHLGTKKESDNIYTFQVPTAYCNMCKQLLEMKN

SEQ ID NO: 135 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETDFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTCAFAANNIRNIIDPEQIKDIVGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRAASESELFIHLTEIIELCLLHADNVPADYWQIYKNKEQCLQRLQPFIAENSDYTNWGDLYVQTDDETPVVHSGIELSVKYGTASLLEKLIASDAKFKITENEFDLWHNARATIEAMIKKREEYHNLWLEAKEKDDNEKKTRIREKNNYARRFVETKGEDYIDICDYIDRYNWLDNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 136 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNRIQIGDGESKRQKALLMWETDFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTCAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSGRYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRAASESELFIHLTEIIELCLLHADNVPADYWQIYKNKEQCLQRLQPFIAENSDYTNWGDLYVQTDDETPVVHSGIELSVKYGTASLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 137 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETDFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRKGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRAASESELFIHLLEIIELCLLHADNVPADYWQIYKNKEQCLQRLQPFIAENSDYTNWGDLYVQTDDETPVVHSGIELSVKYGTASLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 138 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRKKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDIVGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSGRYFLLKLIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRAASESELFIHLTEIIELCLLHADNVPADYWQIYKNKEQCLQRLQPFIAENSDYTNWGDLYVQTDDETPVVHSGIELSVKYGTASLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTEYCNMCKQLLEMKN

SEQ ID NO: 139 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRKKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNIKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRAASESELFIHLLEIIELCLLHADNVPADYWQIYKNKEQCLQRLQPFIAENSDYTNWGDLYVQTDDETPVVHSGIELSVKYGTASLLEKLIASDAKFKITENEFDLWHNARATIEAMIKKREEYHNLWLEAKEKDDNEKKTRIREKNNYARRFVETKGEDYIDICDYIDRYNWLDNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTEYCNMCKQLLEMKN

SEQ ID NO: 140 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETDFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRKGKYQHHDLKADATSENLTNIKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTNYQNSKQDESFYNLELTAYQLKDDFSEEAWSGRYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTASLLEKLIASDAKFKITENEFDLWHNARATIEAMIKKREEYHNLWLEAKEKDDNEKKTRIREKNNYARRFVETKGEDYIDICDYIDRYNWLDNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 141 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETDFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNIKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRAASESELFIHLLEIIELCLLHADNVPADYWQIYKNKEQCLQRLQPFIAENSDYTNWGDLYVQTDDETPVVHSGIELSVKYGTASLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTEYCNMCKQLLEMKN

SEQ ID NO: 142 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRKGKYQHHDLKADATSENLTNLKRDEAFVMNLITTSAFAANNIRNIIDPEQIKDIVGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTNYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRAASESELFIHLTEIIELCLLHADNVPADYWQIYKNKEQCLQRLQPFIAENSDYTNWGDLYVQTDDETPVVHSGIELSVKYGTASLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 143 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELHPYYDWSKWYINDKSGKLKKSITNNHIQIGDGESKRKKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRKGKYQHHDLKADATSENLTNLKRDEAFVMNLITTCAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFSLCRTVVPFAPGFKKVMKGGTNYQNSKQDESFYNLELTAYQLKDDFSEEAWSGRYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRAASESELFIHLTEIIELCLLHADNVPADYWQIYKNKEQCLQRLQPFIAENSDYTNWGDLYVQTDDETPVVHSGIELSVKYGTASLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITSKIIFHLGTKKESDNIYTFQVPTAYCNMCKQLLEMKN

SEQ ID NO: 144 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETDFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRKGKYQHHDLKADATSENLTNLKRDEAFVMNLITTSAFAANNIRNIIDPEQIKDILGKDDFIRSMKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTNYQNSKQDESFYNLELTAYQLKDDFSEEAWSGRYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTASLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 145 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRKKALLMWETDFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRKGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSGRYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRAASESELFIHLTEIIELCLLHADNVPADYWQIYKNKEQCLQRLQPFIAENSDYTNWGDLYVQTDDETPVVHSGIELSVKYGTASLLEKLIASDAKFKITENEFDLWHNARATIEAMIKKREEYHNLWLEAKEKDDNEKKTRIREKNNYARRFVETKGEDYIDICDYIDRYNWLDNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 146 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTASLLEKLIARDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 147 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNHKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTASLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 148 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRLQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTASLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 149 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTASLLEKLIASDAKFKITENEFDLWHNARPTIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 150 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYQNWGDLYVQTDDETPVVHSGIELSVKYGTASLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 151 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDARHLSTLRNELIKYRASSESELFIHLTEIIELCLLHADNVPADYWQIYKNKEQCLQRLQPFIAENSDYTNWGDLYVQTDDETPVVHSGIELSVKYGTASLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 152 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTSNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTASLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 153 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDKETPVVHSGIELSVKYGTASLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 154 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTASLLEKLIASDAKFKITENEFDLWHNARATIEAMIKKREEYHNLWLEAKEKDDNEKKTRIREKNNYARRFVETKGEDYIDICDYIDRYNWLNNKLHFEHLKQLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 155 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTKWGDLYVQTDDETPVVHSGIELSVKYGTASLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 156 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTTVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTASLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 157 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDMTPVVHSGIELSVKYGTASLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 158 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTASLLEKLIASDAKFKITENEFDLWHNARAKIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 159 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTASLLEKLIASDAKFKITENEFDLWHNARATIEAMIKKREEYHNLWLEAKEKDENEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 160 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYRQIYKNKEQCLQRLQPFIAENSDYTNWGDLYVQTDDETPVVHSGIELSVKYGTASLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 161 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDGETPVVHSGIELSVKYGTASLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 162 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYNQIYKNKEQCLQRLQPFIAENSDYTNWGDLYVQTDDETPVVHSGIELSVKYGTASLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 163 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTASLLEKLIASDAKFKITENEFDLWHNARATIEAMIKKREEYHNLWLQAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 164 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDQVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTASLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 165 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTASLLEKLIASDAKFKITEPEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 166 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHTGIELSVKYGTASLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 167 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQRKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTASLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 168 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHRGIELSVKYGTASLLEKLIASDAKFKITENEFDLWHNARATIEAMIKKREEYHNLWLEAKEKDDKEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 169 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTASLLEKLIASDAKFKITENEFDLWHNARATIEAMIKKREEYHNLWLEAKAKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 170 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDGTPVVHSGIELSVKYGTASLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 171 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWMIYKNKEQCLQRLQPFIAENSDYTNWGDLYVQTDDETPVVHSGIELSVKYGTASLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 172 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDSLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTASLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 173 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYENKEQCLQRLQPFIAENSDYTNWGDLYVQTDDETPVVHSGIELSVKYGTASLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 174 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTASLLEKLIASDAKFKITENEFDLWHSARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 175 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTASLLEKLIASDAKFKITENEFDLWHNARATIEAMIKKREEYHNLWLEAKEKDDNEKKTRIREKNNYARRFVETKGEDYIDICDYIDRYNWLNNKLHFEHLKKLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 176 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFAQTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTASLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 177 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTASLLEKLIASDAKFKITENEFDLWHNARATIEAMIKKREMYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 178 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGTRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTAKLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 179 - protein, 1098 aa, synthetic construct
MRVSKVKVGVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTAKLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 180 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTAKLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLLVGSITSKKIFHLGTKKESDNIYTFQVPTAYCNMCKQLLEMKN

SEQ ID NO: 181 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTAKLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHTLQVGSITSKKIFHLGTKKESDNIYTFQVPTAYCNMCKQLLEMKN

SEQ ID NO: 182 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRSNLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTAKLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 183 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYIYDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTAKLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 184 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTAKLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLRYDRKLKNAVAKTFIDLFDKHGMVLKLKFDSQMHQLQVGSITSKKIFHLGTKKESDNIYTFQVPTAYCNMCKQLLEMKN

SEQ ID NO: 185 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWNMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTAKLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 186 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWILEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTAKLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 187 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIKIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTAKLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 188 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSQLRNELIKYRASSESELFIHLTEIIELCLLHADNVPADYWQIYKNKEQCLQRLQPFIAENSDYTNWGDLYVQTDDETPVVHSGIELSVKYGTAKLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 189 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSHNLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTAKLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 190 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSKKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTAKLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 191 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPGDYWQIYKNKEQCLQRLQPFIAENSDYTNWGDLYVQTDDETPVVHSGIELSVKYGTAKLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 192 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQMLIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTAKLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 193 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTAKLLEKLIASDAKFKITENEFDLWHNARATIEAMIKKREEYHNLWLEAKEKDDNEKKTRIREKNNYARRFVETKGEDYIDICDYIDRYNWLENKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 194 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTAKLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFGSQMHQLQVGSITSKKIFHLGTKKESDNIYTFQVPTAYCNMCKQLLEMKN

SEQ ID NO: 195 - protein, 1098 aa, synthetic construct
MRVSKVKVTVNNKDTMVLMHRSAEKGALIYDTIDKKDRTEEIIPQKKQKSFELSIANKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTAKLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 196 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSKVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTAKLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 197 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQQIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTAKLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 198 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTAKLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKVFIDLFDKHGMVLKLKFDSQMHQLQVGSITSKKIFHLGTKKESDNIYTFQVPTAYCNMCKQLLEMKN

SEQ ID NO: 199 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTAKLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIMLFDKHGMVLKLKFDSQMHQLQVGSITSKKIFHLGTKKESDNIYTFQVPTAYCNMCKQLLEMKN

SEQ ID NO: 200 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTAKLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVQTAYCNMCKQLLEMKN

SEQ ID NO: 201 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLTIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTAKLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 202 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDCKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTAKLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 203 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAATIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTAKLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 204 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSLEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTAKLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 205 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWKMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTAKLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 206 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKKKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTAKLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 207 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYRLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTAKLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 208 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTCAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTAKLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITPKIIFHLGTKKESDNIYTFQVPTAYCNMCKQLLEMKN

SEQ ID NO: 209 - protein, 1098 aa, synthetic construct
MRVSKVKVTVNNKDKMVLMHRSAEKGALIYDTIDKKDRTEEIIPQKKQKSFELSIQNKTLLRYEAIKKQKLDRSKYKVVEDIFKYRKLPAKITLEDILPFLNHKFQLPVKYQKEGQWVQFNLSTCILQAVKHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTKQLENAMRAKLLRQGKYQHHDLKADATSENLTNLKRDEAFVMNLITTCAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFLKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTAKLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITPKIIFHLGTKKESDNIYTFQVPTAYCNMCKQLLEMKN

SEQ ID NO: 210 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVKHQNLDELQPYYDWSKWYINDKSGKLKKSITNNHIQIGDGESKRQKALLMWETEFTDKGELDLETYHKKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTKQLENAVRANLLRQGKYQHHDLKADATSENLTNLKRDEAFVMNLITTCAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTAKLLEKLIASDAKFKITENEFDLWHNARATIEAMIKKREELHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITPKIIFHLGTKKESDNIYTFQVPTAYCNMCKQLLEMKN

SEQ ID NO: 211 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTCAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTAKLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEILGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVSKTFIDLFDKHGMVLKLKFDSQMHQLQVGSITSKIIFHLGTKKESDNIYTFQVPTAYCNMCKQLLEMKN

SEQ ID NO: 212 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTKQLENAVRANLLRQGKYQHHDLKADATSENLTNLKRDEAFVMNLITTCAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWNARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQVFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTAKLLEKLIASDAKFKITENEFDLWHNARATIEAMIKKREELHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITSKIIFHLGTKKESDNIYTFQVPTAYCNMCKQLLEMKN

SEQ ID NO: 213 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFQLPVKYQKEGQWVQFELSTCILQAVEHQNLDELQPYYDWSKWYINDKSGKLKKSITNNHIQIGDGESKRQKALLMWETEFTDKGELDLETYHEKYIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTCAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTASLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 214 - protein, 1098 aa, synthetic construct
MRVSKVKVTVNNKDKMVLMHRSAEKGALIYDTIDKKDRTEEIIPQKKQKSFELSIQNKTLLRYEAIKKQKLDRSKYKVVEDIFKYRKLPAKITLEDILPFLNHKFQLPVKYQKEGQWVQFELSTCILQAVKHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAVRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTASLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNLTIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITSKIIFHLGTKKESDNIYTFQVPTAYCNMCKQLLEMKN

SEQ ID NO: 215 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFQLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAVRANLLRQGKYQHHDLKADATSENLTNLKRDEAFVMNLITTCAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQVFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTAKLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVSKTFIDLFDKHGMVLKLKFDSQMHQLQVGSITSKIIFHLGTKKESDNIYTFQVPTAYCNMCKQLLEMKN

SEQ ID NO: 216 - protein, 1098 aa, synthetic construct
MRVSKVKVTVNNKDKMVLMHRSAEKGALIYDTIDKKDRTEEIIPQKKQKSFELSIQNKTLLRYEAIKKQKLDRSKYKVVEDIFKYRKLPAKITLEDILPFLNHKFQLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTKQLENAVRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTASLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNLTIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITPKIIFHLGTKKESDNIYTFQVPTAYCNMCKQLLEMKN

SEQ ID NO: 217 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFQLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAVRANLLRQGKYQHHDLKADATSENLTNLKRDEAFVMNLITTCAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTAKLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 218 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKYIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTCAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQVFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTASLLEKLIASDAKFKITENEFDLWHNARATIEAMIKKREELHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVSKTFIDLFDKHGMVLKLKFDSQMHQLQVGSITPKIIFHLGTKKESDNIYTFQVPTAYCNMCKQLLEMKN

SEQ ID NO: 219 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHKKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTKQLENAMRANLLRQGKYQHHDLKADATSENLTNLKRDEAFVMNLITTCAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTAKLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITSKIIFHLGTKKESDNIYTFQVPTAYCNMCKQLLEMKN

SEQ ID NO: 220 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTKQLENAVRANLLRQGKYQHHDLKADATSENLTNLKRDEAFVMNLITTCAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTAKLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITPKIIFHLGTKKESDNIYTFQVPTAYCNMCKQLLEMKN

SEQ ID NO: 221 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFQLPVKYQKEGQWVQFELSTCILQAVKHQNLDELQPYYDWSKWYINDKSGKLKKSITNNHIQIGDGESKRQKALLMWETEFTDKGELDLETYHEKYIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAVRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTASLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITSKIIFHLGTKKESDNIYTFQVPTAYCNMCKQLLEMKN

SEQ ID NO: 222 - protein, 1098 aa, synthetic construct
MRVSKVKVTVNNKDKMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFNLSTCILQAVKHQNLDELQPYYDWSKWYINDKSGKLKKSITNNHIQIGDGESKRQKALLMWETEFTDKGELDLETYHEKYIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTASLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 223 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTCAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTAKLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHDAYFKRLFLCNNYSDTRNHIAHFNYLTKSAANYSLIDLINQLRELLHYDRKLKNAVAKTFIDLFDKHGMVLKLKFDSQMHQLQVGSITSKIIFHLGTKKESDNIYTFQVPTAYCNMCKQLLEMKN

SEQ ID NO: 224 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFQLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAVRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNHKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDARHLSTLRNELIKYRASSESELFIHLTEIIELCLLHADNVPADYWQIYKNKEQCLQRLQPFIAENSDYTNWGDLYVQTDDETPVVHRGIELSVKYGTAKLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 225 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKYIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCMLLDANHLSTLRNELIKYRASSESELFIHLTEIIELCLLHADNVPADYWQIYKNKEQCLQRLQPFIAENSDYTNWGDLYVQTDKETPVVHSGIELSVKYGTAKLLEKLIASDAKFKITENEFDLWHNARATIEAMIKKREEYHNLWLEAKEKDDKEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 226 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTTVPFAPGFKKVMKGGTGYQNSKQDESFYNLELTAYQRKDDFSEEAWSARYFLLKIIYNYLFLPEFTSNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHRGIELSVKYGTAKLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 227 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKYIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNHKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTAKLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 228 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTSNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDKETPVVHSGIELSVKYGTAKLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 229 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNHKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHRGIELSVKYGTAKLLEKLIASDAKFKITENEFDLWHNARATIEAMIKKREEYHNLWLEAKEKDDNEKKTRIREKNNYARRFVETKGEDYIDICDYIDRYNWLNNKLHFEHLKKLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 230 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFQLPVKYQKEGQWVQFELSTCILQAVEHQNLDELQPYYDWSKWYINDKSGKLKKSITNNHIQIGDGESKRQKALLMWETEFTDKGELDLETYHEKYIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTCAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQRKDDFSEEAWSARYFLLKIIYNYLFLPEFTSNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDKETPVVHSGIELSVKYGTAKLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 231 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAVRANLLRQGKYQHHDLKADATSENLTNLKRDEAFVMNLITTCAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTTVPFAPGFKKVMKGGTGYQNSKQDESFYNLELTAYQLKDDFSEEAWSARYFLLKIIYNYLFLPEFTNNDKVFADTVAWLLGKNRLQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPTKDEQIAFYVYCKLLDARHLSTLRNELIKYRASSESELFIHLTEIIELCLLHADNVPADYNQIYKNKEQCLQRLQPFIAENSDYTNWGDLYVQTDDETPVVHRGIELSVKYGTAKLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 232 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFQLPVKYQKEGQWVQFELSTCILQAVEHQNLDELQPYYDWSKWYINDKSGKLKKSITNNHIQIGDGESKRQKALLMWETEFTDKGELDLETYHEKYIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAVRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTSNHKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTAKLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 233 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTTVPFAPGFKKVMKGGTGYQNSKQDESFYNLELTAYQLKDDFSEEAWSARYFLLKIIYNYLFLPEFTNNHKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYNQIYKNKEQCLQRLQPFIAENSDYTNWGDLYVQTDDETPVVHRGIELSVKYGTAKLLEKLIASDAKFKITENEFDLWHNARATIEAMIKKREEYHNLWLEAKEKDDKEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 234 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTSNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYNQIYKNKEQCLQRLQPFIAENSDYTNWGDLYVQTDKETPVVHRGIELSVKYGTAKLLEKLIASDAKFKITENEFDLWHNARATIEAMIKKREEYHNLWLEAKEKDDNEKKTRIREKNNYARRFVETKGEDYIDICDYIDRYNWLNNKLHFEHLKKLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 235 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYNQIYKNKEQCLQRLQPFIAENSDYTNWGDLYVQTDDETPVVHRGIELSVKYGTAKLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 236 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKYIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTCAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRLQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTAKLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 237 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFQLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAVRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNHKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDKETPVVHRGIELSVKYGTAKLLEKLIASDAKFKITENEFDLWHNARATIEAMIKKREEYHNLWLEAKEKDDNEKKTRIREKNNYARRFVETKGEDYIDICDYIDRYNWLNNKLHFEHLKKLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 238 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDKETPVVHSGIELSVKYGTAKLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDVINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 239 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAVRANLLRQGKYQHHDLKADATSENLTNLKRDEAFVMNLITTCAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTTVPFAPGFKKVMKGGTGYQNSKQDESFYNLELTAYQRKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDKETPVVHRGIELSVKYGTAKLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 240 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAVRANLLRQGKYQHHDLKADATSENLTNLKRDEAFVMNLITTCAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDQVKFHLCRTVVPFAPGFKKVMKGGTGYQNSKQDESFYNLELTAYQLKDDFSEEAWSARYFLLKIIYNYLFLPEFTNNDKVFADTVAWLLGKNRLQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDARHLSTLRNELIKYRASSESELFIHLTEIIELCLLHADNVPADYWQIYKNKEQCLQRLQPFIAENSDYTNWGDLYVQTDKETPVVHRGIELSVKYGTAKLLEKLIASDAKFKITENEFDLWHNARATIEAMIKKREEYHNLWLEAKEKDDNEKKTRIREKNNYARRFVETKGEDYIDICDYIDRYNWLNNKLHFEHLKKLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 241 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYNQIYKNKEQCLQRLQPFIAENSDYTNWGDLYVQTDKETPVVHSGIELSVKYGTAKLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 242 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTTVPFAPGFKKVMKGGTGYQNSKQDESFYNLELTAYQRKDDFSEEAWSARYFLLKIIYNYLFLPEFTNNHKVFADTVAWLLGKNRLQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYNQIYKNKEQCLQRLQPFIAENSDYTNWGDLYVQTDDETPVVHRGIELSVKYGTAKLLEKLIASDAKFKITENEFDLWHNARATIEAMIKKREEYHNLWLEAKEKDDNEKKTRIREKNNYARRFVETKGEDYIDICDYIDRYNWLNNKLHFEHLKKLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 243 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKYIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDANHLSTLRNELIKYRASSESELFIHLTEIIELCLLHADNVPADYWQIYKNKEQCLQRLQPFIAENSDYTNWGDLYVQTDDETPVVHSGIELSVKYGTAKLLEKLIASDAKFKITENEFDLWHNARATIEAMIKKREEYHNLWLEAKEKDDNEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 244 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAMRANLLRQGKYQHHDLKADATSENLTNLKRDEAFVMNLITTSAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTVVPFAPGFKKVMKGGTGYQNSKQDESFYNLELTAYQLKDDFSEEAWSARYFLLKIIYNYLFLPEFTSNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDARHLSTLRNELIKYRASSESELFIHLTEIIELCLLHADNVPADYWQIYKNKEQCLQRLQPFIAENSDYTNWGDLYVQTDDETPVVHRGIELSVKYGTAKLLEKLIASDAKFKITENEFDLWHSARATIEAMIKKREEYHNLWLEAKEKDDKEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 245 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFQLPVKYQKEGQWVQFELSTCILQAVEHQNLDELQPYYDWSKWYINDKSKKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAVRANLLRQGKYQHHDLKADATSENLTNLKRDEAFVMNLITTCAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTTVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQQIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDARHLSTLRNELIKYRASSESELFIHLTEIIELCLLHADNVPGDYNQIYKNKEQCLQRLQPFIAENSDYTNWGDLYVQTDDETPVVHRGIELSVKYGTAKLLEKLIAKDAKFKITENEFDLWHNARATIEAMIKKREEYHNLWLEAKEKDDKEKKTRIREKNNYARRFVETKGEDYIDICDYIDRYNWLNNKLHFEHLKSLQNITIEMLGRMAGFVVLWERDFQYLDQQRVKQQGGALMDFSHGIPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 246 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSKKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAVRANLLRQGKYQHHDLKADATSENLTNLKRDEAFVMNLITTCAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTTVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDARHLSQLRNELIKYRASSESELFIHLTEIIELCLLHADNVPADYNQIYKNKEQCLQRLQPFIAENSDYTNWGDLYVQTDDETPVVHRGIELSVKYGTAKLLEKLIASDAKFKITENEFDLWHNARATIEAMIKKREEYHNLWLEAKEKDDKEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLRYDRKLKNAVAKTFIDLFDKHGMVLKLKFDSQMHQLQVGSITSKKIFHLGTKKESDNIYTFQVPTAYCNMCKQLLEMKN

SEQ ID NO: 247 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFQLPVKYQKEGQWVQFELSTCILQAVEHQNLDELQPYYDWSKWYINDKSKKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAVRANLLRQGKYQHHDLKADATSENLTNLKRDEAFVMNLITTCAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTTVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDARHLSTLRNELIKYRASSESELFIHLTEIIELCLLHADNVPGDYNQIYKNKEQCLQRLQPFIAENSDYTNWGDLYVQTDDETPVVHRGIELSVKYGTAKLLEKLIASDAKFKITENEFDLWHNARATIEAMIKKREEYHNLWLEAKEKDDKEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFGSQMHQLQVGSITSKKIFHLGTKKESDNIYTFQVPTAYCNMCKQLLEMKN

SEQ ID NO: 248 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFQLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAVRANLLRQGKYQHHDLKADATSENLTNLKRDEAFVMNLITTCAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTTVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWNMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDARHLSTLRNELIKYRASSESELFIHLTEIIELCLLHADNVPADYNQIYKNKEQCLQRLQPFIAENSDYTNWGDLYVQTDDETPVVHRGIELSVKYGTAKLLEKLIAKDAKFKITENEFDLWHNARATIEAMIKKREEYHNLWLEAKEKDDKEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLRYDRKLKNAVAKTFIDLFDKHGMVLKLKFDSQMHQLQVGSITSKKIFHLGTKKESDNIYTFQVPTAYCNMCKQLLEMKN

SEQ ID NO: 249 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLTIYNLEVVKYLEHYFPIKSNVRKNSAADIVYYLKSETIKSTVTHQLENAVRANLLRQGKYQHHDLKADATSENLTNLKRDEAFVMNLITTCAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTTVPFAPGFKKVMKGGTGYQNSKQDESFYNLELTAYQLKDDFSLEAWSARYFLLKIIYNYLFLPEFTNNDKVFADTVAWLLGKNRQQAQSSKNKNAYAFADIRLMESSESITDYMAYVQSQWNMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDARHLSTLRNELIKYRASSESELFIHLTEIIELCLLHADNVPADYNQIYKNKEQCLQRLQPFIAENSDYTNWGDLYVQTDRETPVVHRGIELSVKYGTAKLLEKLIAKDAKFKITENEFDLWHNARATIEAMIKKREEYHNLWLEAKEKDDKEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLRYDRKLKNAVAKTFIDLFDKHGMVLKLKFDSQMHQLQVGSITSKKIFHLGTKKESDNIYTFQVPTAYCNMCKQLLEMKN

SEQ ID NO: 250 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFQLPVKYQKEGQWVQFELSTCILQAVEHQNLDELQPYYDWSKWYINDKSKKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAVRANLLRQGKYQHHDLKADATSENLTNLKRDEAFVMNLITTCAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTTVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKKKALEKKICPFCHIQNNKIDPAKDEQIAFYVYCKLLDARHLSTLRNELIKYRASSESELFIHLTEIIELCLLHADNVPADYNQIYKNKEQCLQRLQPFIAENSDYTNWGDLYVQTDDETPVVHRGIELSVKYGTAKLLEKLIAKDAKFKITENEFDLWHNARATIEAMIKKREEYHNLWLEAKEKDDKEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLRYDRKLKNAVAKTFIDLFDKHGMVLKLKFDSQMHQLQVGSITSKKIFHLGTKKESDNIYTFQVPTAYCNMCKQLLEMKN

SEQ ID NO: 251 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAVRANLLRQGKYQHHDLKADATSENLTNLKRDEAFVMNLITTCAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTTVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDARHLSTLRNELIKYRASSESELFIHLTEIIELCLLHADNVPGDYNQIYKNKEQCLQRLQPFIAENSDYTNWGDLYVQTDDETPVVHRGIELSVKYGTAKLLEKLIAKDAKFKITENEFDLWHNARATIEAMIKKREEYHNLWLEAKEKDDKEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 252 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAVRANLLRQGKYQHHDLKADATSENLTNLKRDEAFVMNLITTCAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTTVPFAPGFKKVMKGGTGYQNSKQDESFYNLELTAYQLKDDFSLEAWSARYFLLKIIYNYLFLPEFTNNDKVFADTVAWLLGKNRQQAQSSKNKNAYAFADIRLMESSESITDYMAYVQSQWNMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDARHLSTLRNELIKYRASSESELFIHLTEIIELCLLHADNVPADYNQIYKNKEQCLQRLQPFIAENSDYTNWGDLYVQTDRETPVVHRGIELSVKYGTAKLLEKLIAKDAKFKITENEFDLWHNARATIEAMIKKREEYHNLWLEAKEKDDKEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 253 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAVRANLLRQGKYQHHDLKADATSENLTNLKRDEAFVMNLITTCAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTTVPFAPGFKKVMKGGTGYQNSKQDESFYNLELTAYQLKDDFSLEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDARHLSTLRNELIKYRASSESELFIHLTEIIELCLLHADNVPADYNQIYKNKEQCLQRLQPFIAENSDYTNWGDLYVQTDDETPVVHRGIELSVKYGTAKLLEKLIAKDAKFKITENEFDLWHNARATIEAMIKKREEYHNLWLEAKEKDDKEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 254 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAVRANLLRQGKYQHHDLKADATSENLTNLKRDEAFVMNLITTCAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTTVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWNMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDARHLSTLRNELIKYRASSESELFIHLTEIIELCLLHADNVPADYNQIYKNKEQCLQRLQPFIAENSDYTNWGDLYVQTDDETPVVHRGIELSVKYGTAKLLEKLIASDAKFKITENEFDLWHNARATIEAMIKKREEYHNLWLEAKEKDDKEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 255 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNLRKNSAADIVYYLKSETIKSTVTHQLENAVRANLLRQGKYQHHDLKADATSENLTNLKRDEAFVMNLITTCAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTTVPFAPGFKKVMKGGTGYQNSKQDESFYNLELTAYQLKDDFSLEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDARHLSTLRNELIKYRASSESELFIHLTEIIELCLLHADNVPADYNQIYKNKEQCLQRLQPFIAENSDYTNWGDLYVQTDDETPVVHRGIELSVKYGTAKLLEKLIAKDAKFKITENEFDLWHNARATIEAMIKKREEYHNLWLEAKEKDDKEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKVFIDLFDKHGMVLKLKFDSQMHQLQVGSITSKKIFHLGTKKESDNIYTFQVPTAYCNMCKQLLEMKN

SEQ ID NO: 256 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFQLPVKYQKEGQWVQFELSTCILQAVEHQNLDELQPYYDWSKWYINDKSKKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAVRANLLRQGKYQHHDLKADATSENLTNLKRDEAFVMNLITTCAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTTVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWNMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDARHLSQLRNELIKYRASSESELFIHLTEIIELCLLHADNVPADYNQIYKNKEQCLQRLQPFIAENSDYTNWGDLYVQTDDETPVVHRGIELSVKYGTAKLLEKLIAKDAKFKITENEFDLWHNARATIEAMIKKREEYHNLWLEAKEKDDKEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFGSQMHQLQVGSITSKKIFHLGTKKESDNIYTFQVPTAYCNMCKQLLEMKN

SEQ ID NO: 257 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAVRANLLRQGKYQHHDLKADATSENLTNLKRDEAFVMNLITTCAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTTVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDARHLSTLRNELIKYRASSESELFIHLTEIIELCLLHADNVPADYNQIYKNKEQCLQRLQPFIAENSDYTNWGDLYVQTDDETPVVHRGIELSVKYGTAKLLEKLIAKDAKFKITENEFDLWHNARATIEAMIKKREEYHNLWLEAKEKDDKEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 258 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFQLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLTIYNLEVVKYLEHYFPIKSNVRKNSAADIVYYLKSETIKSTVTHQLENAVRANLLRQGKYQHHDLKADATSENLTNLKRDEAFVMNLITTCAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTTVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDARHLSTLRNELIKYRASSESELFIHLTEIIELCLLHADNVPGDYNQIYKNKEQCLQRLQPFIAENSDYTNWGDLYVQTDDETPVVHRGIELSVKYGTAKLLEKLIASDAKFKITENEFDLWHNARATIEAMIKKREEYHNLWLEAKEKDDKEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 259 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAVRANLLRQGKYQHHDLKADATSENLTNLKRDEAFVMNLITTCAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTTVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWNMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDARHLSTLRNELIKYRASSESELFIHLTEIIELCLLHADNVPGDYNQIYKNKEQCLQRLQPFIAENSDYTNWGDLYVQTDDETPVVHRGIELSVKYGTAKLLEKLIASDAKFKITENEFDLWHNARATIEAMIKKREEYHNLWLEAKEKDDKEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 260 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAVRANLLRQGKYQHHDLKADATSENLTNLKRDEAFVMNLITTCAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTTVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDARHLSTLRNELIKYRASSESELFIHLTEIIELCLLHADNVPADYNQIYKNKEQCLQRLQPFIAENSDYTNWGDLYVQTDRETPVVHRGIELSVKYGTAKLLEKLIAKDAKFKITENEFDLWHNARATIEAMIKKREEYHNLWLEAKEKDDKEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 261 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFQLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLTIYNLEVVKYLEHYFPIKSNVRKNSAADIVYYLKSETIKSTVTHQLENAVRANLLRQGKYQHHDLKADATSENLTNLKRDEAFVMNLITTCAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTTVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWNMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDARHLSQLRNELIKYRASSESELFIHLTEIIELCLLHADNVPGDYNQIYKNKEQCLQRLQPFIAENSDYTNWGDLYVQTDDETPVVHRGIELSVKYGTAKLLEKLIAKDAKFKITENEFDLWHNARATIEAMIKKREEYHNLWLEAKEKDDKEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLRYDRKLKNAVAKTFIDLFDKHGMVLKLKFDSQMHQLQVGSITSKKIFHLGTKKESDNIYTFQVPTAYCNMCKQLLEMKN

SEQ ID NO: 262 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAVRANLLRQGKYQHHDLKADATSENLTNLKRDEAFVMNLITTCAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTTVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDARHLSQLRNELIKYRASSESELFIHLTEIIELCLLHADNVPGDYNQIYKNKEQCLQRLQPFIAENSDYTNWGDLYVQTDDETPVVHRGIELSVKYGTAKLLEKLIASDAKFKITENEFDLWHNARATIEAMIKKREEYHNLWLEAKEKDDKEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 263 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFQLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAVRANLLRQGKYQHHDLKADATSENLTNLKRDEAFVMNLITTCAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTTVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDARHLSTLRNELIKYRASSESELFIHLTEIIELCLLHADNVPGDYNQIYKNKEQCLQRLQPFIAENSDYTNWGDLYVQTDDETPVVHRGIELSVKYGTAKLLEKLIASDAKFKITENEFDLWHNARATIEAMIKKREEYHNLWLEAKEKDDKEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKVFIDLFDKHGMVLKLKFDSQMHQLQVGSITSKKIFHLGTKKESDNIYTFQVPTAYCNMCKQLLEMKN

SEQ ID NO: 264 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAVRANLLRQGKYQHHDLKADATSENLTNLKRDEAFVMNLITTCAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTTVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDARHLSTLRNELIKYRASSESELFIHLTEIIELCLLHADNVPADYNQIYKNKEQCLQRLQPFIAENSDYTNWGDLYVQTDDETPVVHRGIELSVKYGTAKLLEKLIASDAKFKITENEFDLWHNARATIEAMIKKREEYHNLWLEAKEKDDKEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLRYDRKLKNAVAKTFIDLFDKHGMVLKLKFDSQMHQLQVGSITSKKIFHLGTKKESDNIYTFQVPTAYCNMCKQLLEMKN

SEQ ID NO: 265 - protein, 1098 aa, synthetic construct; identical to SEQ ID NO: 257

SEQ ID NO: 266 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFQLPVKYQKEGQWVQFELSTCILQAVEHQNLDELQPYYDWSKWYINDKSKKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAVRANLLRQGKYQHHDLKADATSENLTNLKRDEAFVMNLITTCAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTTVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDARHLSQLRNELIKYRASSESELFIHLTEIIELCLLHADNVPADYNQIYKNKEQCLQRLQPFIAENSDYTNWGDLYVQTDRETPVVHRGIELSVKYGTAKLLEKLIASDAKFKITENEFDLWHNARATIEAMIKKREEYHNLWLEAKEKDDKEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 267 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSKKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLTIYNLEVVKYLEHYFPIKSNVRKNSAADIVYYLKSETIKSTVTHQLENAVRANLLRQGKYQHHDLKADATSENLTNLKRDEAFVMNLITTCAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTTVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDARHLSQLRNELIKYRASSESELFIHLTEIIELCLLHADNVPADYNQIYKNKEQCLQRLQPFIAENSDYTNWGDLYVQTDDETPVVHRGIELSVKYGTAKLLEKLIASDAKFKITENEFDLWHNARATIEAMIKKREEYHNLWLEAKEKDDKEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKVFIDLFDKHGMVLKLKFDSQMHQLQVGSITSKKIFHLGTKKESDNIYTFQVPTAYCNMCKQLLEMKN

SEQ ID NO: 268 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSKKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAVRANLLRQGKYQHHDLKADATSENLTNLKRDEAFVMNLITTCAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTTVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDARHLSTLRNELIKYRASSESELFIHLTEIIELCLLHADNVPADYNQIYKNKEQCLQRLQPFIAENSDYTNWGDLYVQTDDETPVVHRGIELSVKYGTAKLLEKLIASDAKFKITENEFDLWHNARATIEAMIKKREEYHNLWLEAKEKDDKEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 269 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLTIYNLEVVKYLEHYFPIKSNVRKNSAADIVYYLKSETIKSTVTHQLENAVRANLLRQGKYQHHDLKADATSENLTNLKRDEAFVMNLITTCAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTTVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQQIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDARHLSTLRNELIKYRASSESELFIHLTEIIELCLLHADNVPADYNQIYKNKEQCLQRLQPFIAENSDYTNWGDLYVQTDRETPVVHRGIELSVKYGTAKLLEKLIAKDAKFKITENEFDLWHNARATIEAMIKKREEYHNLWLEAKEKDDKEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 270 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFQLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAVRANLLRQGKYQHHDLKADATSENLTNLKRDEAFVMNLITTCAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTTVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDARHLSTLRNELIKYRASSESELFIHLTEIIELCLLHADNVPADYNQIYKNKEQCLQRLQPFIAENSDYTNWGDLYVQTDDETPVVHRGIELSVKYGTAKLLEKLIASDAKFKITENEFDLWHNARATIEAMIKKREEYHNLWLEAKEKDDKEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLRYDRKLKNAVAKTFIDLFDKHGMVLKLKFDSQMHQLQVGSITSKKIFHLGTKKESDNIYTFQVPTAYCNMCKQLLEMKN

SEQ ID NO: 271 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSKKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAVRANLLRQGKYQHHDLKADATSENLTNLKRDEAFVMNLITTCAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTTVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDARHLSTLRNELIKYRASSESELFIHLTEIIELCLLHADNVPADYNQIYKNKEQCLQRLQPFIAENSDYTNWGDLYVQTDRETPVVHRGIELSVKYGTAKLLEKLIAKDAKFKITENEFDLWHNARATIEAMIKKREEYHNLWLEAKEKDDKEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKVFIDLFDKHGMVLKLKFDSQMHQLQVGSITSKKIFHLGTKKESDNIYTFQVPTAYCNMCKQLLEMKN

SEQ ID NO: 272 - protein, 1098 aa, synthetic construct; identical to SEQ ID NO: 245

SEQ ID NO: 273 - protein, 1098 aa, synthetic construct; identical to SEQ ID NO: 251

SEQ ID NO: 274 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAVRANLLRQGKYQHHDLKADATSENLTNLKRDEAFVMNLITTCAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTTVPFAPGFKKVMKGGTGYQNSKQDESFYNLELTAYQLKDDFSLEAWSARYFLLKIIYNYLFLPEFTNNDKVFADTVAWLLGKNRQQAQSSKNKNAYAFADIRLMESSESITDYMAYVQSQWNMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDARHLSTLRNELIKYRASSESELFIHLTEIIELCLLHADNVPADYNQIYKNKEQCLQRLQPFIAENSDYTNWGDLYVQTDRETPVVHRGIELSVKYGTAKLLEKLIAKDAKFKITENEFDLWHNARATIEAMIKKREEYHNLWLEAKEKDDKEKKTRIREKNNYARRFVETKGEDYIDICDYIDRYNWLNNKLHFEHLKKLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 275 - protein, 1098 aa, synthetic construct; identical to SEQ ID NO: 254

SEQ ID NO: 276 - protein, 1098 aa, synthetic construct; identical to SEQ ID NO: 271

SEQ ID NO: 277 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAVRANLLRQGKYQHHDLKADATSENLTNLKRDEAFVMNLITTCAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTTVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQQIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDARHLSTLRNELIKYRASSESELFIHLTEIIELCLLHADNVPADYNQIYKNKEQCLQRLQPFIAENSDYTNWGDLYVQTDRETPVVHRGIELSVKYGTAKLLEKLIAKDAKFKITENEFDLWHNARATIEAMIKKREEYHNLWLEAKEKDDKEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLRYDRKLKNAVAKTFIDLFDKHGMVLKLKFDSQMHQLQVGSITSKKIFHLGTKKESDNIYTFQVPTAYCNMCKQLLEMKN

SEQ ID NO: 278 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFQLPVKYQKEGQWVQFELSTCILQAVEHQNLDELQPYYDWSKWYIDDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLTIYNLEVVKYLEHYFPIKSNVRKNSAADIVYYLKSETIKSTVTHQLENAVRANLLRQGKYQHHDLKADATSENLTNLKRDEAFVMNLITTCAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTTVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDARHLSQLRNELIKYRASSESELFIHLTEIIELCLLHADNVPADYNQIYKNKEQCLQRLQPFIAENSDYTNWGDLYVQTDDETPVVHRGIELSVKYGTAKLLEKLIASDAKFKITENEFDLWHNARATIEAMIKKREEYHNLWLEAKEKDDKEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLRYDRKLKNAVAKVFIDLFDKHGMVLKLKFDSQMHQLQVGSITSKKIFHLGTKKESDNIYTFQVPTAYCNMCKQLLEMKN

SEQ ID NO: 279 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSKKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAVRANLLRQGKYQHHDLKADATSENLTNLKRDEAFVMNLITTCAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTTVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDARHLSTLRNELIKYRASSESELFIHLTEIIELCLLHADNVPADYNQIYKNKEQCLQRLQPFIAENSDYTNWGDLYVQTDRETPVVHRGIELSVKYGTAKLLEKLIASDAKFKITENEFDLWHNARATIEAMIKKREEYHNLWLEAKEKDDKEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLRYDRKLKNAVAKTFIDLFDKHGMVLKLKFDSQMHQLQVGSITSKKIFHLGTKKESDNIYTFQVPTAYCNMCKQLLEMKN

SEQ ID NO: 280 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFQLPVKYQKEGQWVQFELSTCILQAVEHQNLDELQPYYDWSKWYINDKSKKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAVRANLLRQGKYQHHDLKADATSENLTNLKRDEAFVMNLITTCAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTTVPFAPGFKKVMKGGTGYQNSKQDESFYNLELTAYQLKDDFSEEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDARHLSTLRNELIKYRASSESELFIHLTEIIELCLLHADNVPGDYNQIYKNKEQCLQRLQPFIAENSDYTNWGDLYVQTDDETPVVHRGIELSVKYGTAKLLEKLIASDAKFKITENEFDLWHNARATIEAMIKKREEYHNLWLEAKEKDDKEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLRYDRKLKNAVAKTFIDLFDKHGMVLKLKFDSQMHQLQVGSITSKKIFHLGTKKESDNIYTFQVPTAYCNMCKQLLEMKN

SEQ ID NO: 281 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSGKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAVRANLLRQGKYQHHDLKADATSENLTNLKRDEAFVMNLITTCAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTTVPFAPGFKKVMKGGTGYQNSKQDESFYNLELTAYQLKDDFSLEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDARHLSQLRNELIKYRASSESELFIHLTEIIELCLLHADNVPADYNQIYKNKEQCLQRLQPFIAENSDYTNWGDLYVQTDDETPVVHRGIELSVKYGTAKLLEKLIASDAKFKITENEFDLWHNARATIEAMIKKREEYHNLWLEAKEKDDKEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLHYDRKLKNAVAKTFIDLFDKHGMVLKLKFDSQMHQLQVGSITSKKIFHLGTKKESDNIYTFQVPTAYCNMCKQLLEMKN

SEQ ID NO: 282 - protein, 1098 aa, synthetic construct
MRVSKVKVTVNNKDTMVLMHRSAEKGALIYDTIDKKDRTEEIIPQKKQKSFELSIQNKTLLRYEAIKKQKLDRSKYKVVEDIFKYRKLPAKITLEDILPFLNHKFHLPVKYQKEGQWVQFELSTCILQAVEHQNLDELQPYYDWSKWYINDKSKKLKKSITNNHIQIGDGESKRQKALLMWETEFTDKGELDLETYHEKFIIDTLAASLRSVEKGELRDNGTPKETNSYHRNLKKVLQNHQALIFGRRETPNQENRDNKQLAIYNLEVVKYLEHYFPIKSNVRKNSAADIVYYLKSETIKSTVTHQLENAVRANLLRQGKYQHHDLKADATSENLTNLKRDEAFVMNLITTCAFAANNIRNIIDPEQIKDILGKDDFIRSLKKDAFNKHIYGLFFPVGAEMIEPVSLWAMRGAVQQIRNNVAHYKKDALATIFNITKFEYPGAENILFSNTIYKQFFEKELEQLPEAFALQLKSGGVLAQYGFDQLKFLLDVVKFHLCRTTVPFAPGFKKVMKGGTGYQNSKQDESFYNLELTAYQLKDDFSLEAWSARYFLLKIIYNYLFLPEFTNNDKVFADTVAWLLGKNRQQAQSSKNKNAYAFADIRLMESSESITDYMAYVQSQWIMEENKKEDDKKEDTRINFEKFVLQLFVKGFDTFMKQSAYQFINTPQPQINPTDTNQQQADKLKALEKKICPFCHIQNNKIDPAKDEQIAFYVYCKLLDARHLSQLRNELIKYRASSESELFIHLTEIIELCLLHADNVPADYNQIYKNKEQCLQRLQPFIAENSDYTNWGDLYVQTDDETPVVHRGIELSVKYGTAKLLEKLIASDAKFKITENEFDLWHNARATIEAMIKKREEYHNLWLEAKEKDDKEKKTRIREKNNYARRFVETKGEDYIDICDYIDRYNWLNNKLHFEHLKSLQNITIEMLGRMAGFVVLWERDFQYLDQQRVKQQGGALMDFSHGVPKYEELKPHYAYFKRLFLCNNYSDTRNHIAHFNYLTKSAANYSLIDLINQLRELLRYDRKLKNAVAKTFIDLFDKHGMVLKLKFDSQMHQLQVGSITSKKIFHLGTKKESDNIYTFQVPTAYCNMCKQLLEMKN

SEQ ID NO: 283 - RNA, 30 nt, synthetic construct
  source 1..30: mol_type = other RNA
gattatcccaatattgaagggaactacaac